(19) United States
(12) Patent Application Publication
(10) Pub. No.: US 2024/0158760 A1
(43) Pub. Date: May 16, 2024

(54) SYNTHETIC PHAGE WITH RECOMBINANT TAIL-SPIKE PROTEINS AND RELATED METHODS

(71) Applicant: Laboratory Corporation of America Holdings, Burlington, NC (US)

(72) Inventors: Stephen Erickson, White Bear Township, MN (US); Jose S. Gil, Winnetka, CA (US); Matthew J. Brown, Burlington, NC (US); John Paulson, Minneapolis, MN (US)

(73) Assignee: Laboratory Corporation of America Holdings, Burlington, NC (US)

(21) Appl. No.: 18/506,364

(22) Filed: Nov. 10, 2023

Related U.S. Application Data
(60) Provisional application No. 63/383,237, filed on Nov. 10, 2022.
(60) Provisional application No. 63/386,685, filed on Dec. 9, 2022.

(51) Int. Cl. C12N 7/00; C07K 14/005; G01N 33/68

(52) U.S. Cl. CPC C12N 7/00; C07K 14/005; G01N 33/68; C12N 2795/00022

(57) ABSTRACT

Described are compositions, methods, systems, and kits related to related to synthetic phages with a customized host range. Customized host range of the synthetic phage is imparted on the synthetic phage by one or more recombinant tail-spike proteins. For example, a recombinant tail-spike protein may include a combination of the N-terminal region and the C-terminal region is engineered in a laboratory.

### CROSS-REFERENCE TO RELATED APPLICATIONS

This application claims the benefit of priority to U.S. Provisional Patent Application No. 63/383,237, filed Nov. 10, 2022, and to U.S. Provisional Patent Application No. 63/386,685, filed Dec. 9, 2022, the entire contents of each of which are incorporated by reference herein.

### BACKGROUND

There is a strong interest in using phages for various applications related to detection of microorganisms in biological, food, water, and clinical samples, as well as for anti-microbial therapeutic applications, especially when drug-resistant microorganisms are involved. The limited range of hosts for a single phage often limits its usefulness for both diagnostics and therapeutic applications. Traditionally, combinations of various phages, or “cocktails,” were used to address the problem of narrow host range of phages. However, the use of multi-phage combinations may complicate interpretation of test results and overall effectiveness of phage-based applications. The presence of multiple distinct phages in a cocktail may also increase the complexity of reagent preparation, leading to burdensome quality control requirements for regulatory approval. The ability to control host range of a phage to include or exclude a target host of interest can be advantageous.

### BRIEF SUMMARY

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. Some of the exemplary embodiments of the present invention are discussed below.

Included among the embodiments of the present invention and described in the present disclosure are, among others, the following non-limiting exemplary embodiments. An exemplary embodiment of the present invention is a synthetic phage comprising at least one recombinant tail-spike protein (TSP) comprising an N-terminal region and a C-terminal region, wherein (a) a combination of the N-terminal region and at least a part of the C-terminal region is engineered in a laboratory, and/or (b) the C-terminal region comprises one or more engineered amino acid sequences, wherein the synthetic phage is constructed from a parent phage that is an Ackermannviridae phage. In some embodiments of the synthetic phage, the C-terminal region is capable of recognizing a target host. In some embodiments of the synthetic phage, the at least one recombinant TSP confers on the synthetic phage an ability to recognize the target host, wherein the ability to recognize the target host was absent in the parent phage. In some embodiments of the synthetic phage, the C-terminal region of at least one recombinant TSP comprises at least one amino acid sequence occurring in a C-terminal region of a TSP from a phage different from the parent phage and capable of recognizing the target host. In some embodiments of the synthetic phage, the phage different from the parent phage is a non-Ackermannviridae phage. In some embodiments of the synthetic phage, the C-terminal region of at least one recombinant TSP comprises at least one amino acid sequence occurring in a C-terminal region of a TSP in a phage that is not capable of infecting the at least one host recognized by the parent phage. In some embodiments of the synthetic phage, the parent phage is a recombinant phage. In some embodiments of the synthetic phage, the synthetic phage comprises the at least one recombinant TSP is a plurality of recombinant TSPs. In some embodiments of the synthetic phage, the synthetic phage comprises at least two recombinant TSPs capable of recognizing at least two different target hosts. In some embodiments of the synthetic phage, at least one recombinant TSP has at least 70%, at least 75%, at least 80%, at least 85%, at least 90%, at least 95%, or at least 99% sequence similarity to SEQ ID s 6, 7, 8, 10, 12, 14, or 18.

Included among the embodiments of the present invention are recombinant tail-spike proteins (TSPs) comprising an N-terminal region comprising amino acid sequences derived from an Ackermannviridae phage and a C-terminal region, wherein: (a) a combination of the N-terminal region and at least a part of the C-terminal region is engineered in a laboratory, and/or (b) the C-terminal region comprises one or more engineered amino acid sequences. In some embodiments, the C-terminal region of the recombinant TSP is capable of recognizing a target host. In some embodiments of the recombinant TSP, the amino acid sequences of the C-terminal region and the N-terminal region are derived from same Ackermannviridae phage. In some embodiments of the recombinant TSP, the amino acid sequences of the C-terminal region and the N-terminal region are derived from different phages. In some embodiments of the recombinant TSP, the amino acid sequences of the C-terminal region are derived from a non-Ackermannviridae phage. In some embodiments of the recombinant TSP, an amino acid sequence of the C-terminal region comprises one or more amino acid sequences of a TSP of SPTD1 phage. In some embodiments of the recombinant TSP, the recombinant TSP has at least 70%, at least 75%, at least 80%, at least 85%, at least 90%, at least 95%, or at least 99% sequence similarity to SEQ ID s 6, 7, 8, 10, 12, 14, or 18. Also included among the embodiments of the present invention are nucleic acid sequence encoding the recombinant TSPs according to the present disclosure.

Included among the embodiments of the present invention are methods of altering a phage host range. An exemplary method comprises altering a parent phage that is an Ackermannviridae phage to include at least one recombinant tail-spike protein (TSP) according to the present disclosure, thereby generating a synthetic phage with an altered host range. In some embodiments, the parent phage is capable of recognizing at least one host and the synthetic phage with the altered host range is not capable of recognizing the at least one host. In some embodiments, the parent phage is capable of recognizing at least one host, and the synthetic phage with the altered host range is capable of recognizing at least one target host different from the at least one host. In some embodiments, the altered host range is broader or narrower than a host range of the parent phage. In some embodiments, the altering the parent phage comprises altering a C-terminal region of at least one tail-spike protein (TSP) of the parent phage. In some embodiments, the altering the C-terminal region of at least TSP of the parent phage comprises exchanging the at least a part of the C-terminal region of the TSP of the parent phage for the C-terminal region of the recombinant TSP. In some embodiments, the exchanging is performed using homologous recombination.

Included among the embodiments of the present invention are methods of detecting the target host using the synthetic phages according to the present disclosure. In some embodiments, such methods include the steps of contacting a sample with the synthetic phage for a time sufficient for the synthetic phage to infect the target host; and, detecting the synthetic phage or progeny phage of the synthetic phage, wherein positive detection of the synthetic phage or the progeny phage of the synthetic phage indicates that the target host is present in the sample. In some embodiments, the synthetic phage comprises an indicator gene, and the detecting comprises detecting an indicator protein product produced by the synthetic phage or the progeny phage of the synthetic phage, wherein positive detection of the indicator protein product indicates that the target host is present in the sample.

Included among the embodiments of the present invention are kits and systems for performing the methods of the present disclosure, wherein the kits or the system comprise the synthetic phage. Also included among the embodiments of the present invention are methods of detecting the target host using the recombinant TSP according to the present disclosure. Included among the embodiments of the present invention are methods of controlling a microorganism using the synthetic phages according to the present disclosure. Such methods may include a step of administering to a subject or contacting a sample, an object, an apparatus, or a material with the synthetic phage, wherein the synthetic phage is lytic. In any of the methods according to the present disclosure, the sample may be (but is not limited to) a food, environmental, water, commercial, or clinical sample.

Included among the embodiments of the present invention are methods of constructing a synthetic phage comprising: selecting a parent phage capable of infecting a first microorganism and not capable of infecting the second microorganism, wherein the parent phage is an is an Ackermannviridae phage; transforming the first microorganism with a homologous recombination (HR) plasmid comprising a nucleic acid sequence encoding a C-terminal region of a tail-spike protein (TSP) capable of recognizing the second microorganism and HR sequences flanking the nucleic acid sequence complementary to corresponding sequences in a TSP of the parent phage, thereby generating a transformed microorganism; infecting the first microorganism with the parent phage, allowing HR to occur between the HR plasmid and genome of the parent phage, thereby generating a plurality of synthetic phage clones; and, isolating a clone of the synthetic phage comprising the recombinant TSP, thereby constructing the synthetic phage capable of infecting the second microorganism.

Included among the embodiments of the present invention are methods for constructing a synthetic phage with desired host-recognition capacity, comprising the steps of: (a) providing virions of a recombinant phage constructed from an Ackermannviridae phage, wherein the virions lack tail-spike protein-encoding genes (TSP-encoding genes) and comprise tail-spike proteins (TSPs) capable of recognizing a bacterial host; (b) providing the bacterial host comprising one or more plasmids encoding TSPs having the desired host-recognition capacity; (c) infecting the bacterial host with the virions of the recombinant phage; and, (d) allowing progeny phage to be produced in the bacterial host, wherein virions of the progeny phage lack TSP-encoding genes and comprise TSPs encoded by the one or more plasmids in the bacterial hosts, wherein the progeny phage is the synthetic phage with the desired host-recognition capacity. In the embodiments of the above methods, step (a) may further comprise: selecting a parent phage capable of infecting a bacterial host suitable for conducting homologous recombination (HR), wherein the parent phage is the Ackermannviridae phage or an engineered phage constructed from the Ackermannviridae phage; transforming the bacterial host suitable for conducting HR with an HR plasmid comprising a nucleic acid sequence encoding a tractable marker and sequences flanking the nucleic acid sequence complementary to corresponding sequences flanking a cluster of the TSP-encoding genes of the parent phage; infecting the bacterial host suitable for conducting HR with the parent phage, allowing HR to occur between the HR plasmid and genome of the parent phage, thereby generating a plurality of phage clones; and, isolating from the plurality of the phage clones a phage clone comprising the tractable marker, thereby constructing the recombinant phage lacking TSP-encoding genes.

These and other embodiments of the disclosure are described in detail below. For example, some other embodiments are directed to systems, devices, and computer readable media associated with methods described herein.

### BRIEF DESCRIPTION OF THE DRAWINGS

The present disclosure includes the following figures. The figures are intended to illustrate certain embodiments and/or features of the embodiments of the present invention, and to supplement any description(s) of the embodiments of the present invention. The figures do not limit the scope of the embodiments of the present invention, unless the written description expressly indicates that such is the case.

FIG. 1 is a schematic depiction of the four tail-spike proteins (TSPs) of phage CBA120, a member of the Ackermannviridae family. Each of TSPs 1-4 recognizes a unique bacterial host, as indicated. The TSPs form a complex with TSP4 attaching to the baseplate.

FIG. 2 is a schematic illustration of TSPs for an exemplary phage in the Ackermannviridae family with four TSPs. Each TSP is capable of recognizing a different surface host receptor, typically allowing infection of all hosts that display a particular receptor.

FIG. 3 is a schematic illustration of the use of an Ackermannviridae phage as a customizable modular platform for constructing conditionally replicative synthetic phages for detection of Gram-negative bacteria.

FIG. 4 is a schematic depiction of a complementing bacterial host expressing TSPs in trans via a plasmid to complement a recombinant phage lacking TSP-encoding genes, resulting in production of viable infectious recombinants, which are then capable of a single round of infection in wild-type bacteria. The use of different plasmids encoding for unique TSPs allows for the assembly of recombinants with customizable host ranges. Wild-type bacteria lacking the complementing plasmid cannot support the production of infectious progeny due to the lack of TSPs.

FIG. 5 are electron microscopy images of SPTD1 and CBA120 phages. Both phages are members of the Ackermannviridae family.

FIG. 6 is a schematic depiction of a virion of an Ackermannviridae phage.

FIG. 7 is a schematic illustration of homologous recombination process used to generate synthetic phage CBA120-SPTD1.chiTSP2 (RBP-CBA120-2) from CBA120 phage by exchanging CBA120 TSP2 with a recombinant TSP (“CBA120-SPTD1 TSP2”) containing a N-terminal domain from CBA TSP2 and a C-terminal domain from SPTD1 TSP2.

FIG. 8 illustrates a pairwise alignment, performed on EMBOSS Needle tool available from European Molecular Biology Laboratory (EMBL) European Bioinformatics Institute “EMBOSS Needle”), of amino acid sequences of CBA120 TSP2 (SEQ ID:9) and recombinant CBA120-SPTD1 TSP2 (SEQ ID:10). The “neck” region is labelled and indicated with a box. As illustrated, the two amino acid sequences are identical until two amino acids upstream of the neck (as indicated by an arrow).

FIG. 9 shows, in the left panel, an alignment of amino acid sequences around the joining (“splice”) site (indicated by an arrow between amino acids 244 and 245) of CBA120 TSP2 (amino acids (aa) 241-272 of SEQ ID:9) and recombinant CBA120-SPTD1 TSP2 (amino acids (aa) 241-272 of SEQ ID:10). In the right panel, is a schematic illustration of the hypothetical structure of recombinant CBA120-SPTD1 TSP2 protein.

FIG. 10 illustrates pairwise alignment, performed on EMBOSS Needle, of amino acid sequences from SPTD1 TSP2 (SEQ ID:3) and recombinant CBA120-SPTD1 TSP2 (SEQ ID:10) protein. The estimated “neck” region is labelled and indicated with a box.

FIG. 11 is a schematic illustration of HR.CBA120-SPTD1.chimeric.TSP2 plasmid insert.

FIG. 12 are photographic images illustrating the results of the plating experiment testing the properties of synthetic phage CBA120-SPTD1.chiTSP2 (RBP-CBA120-2; also labelled as ChiTSP2) compared to the CBA120 and SPTD1 (also labeled as MTSP1) phages. Culture media alone was also tested as a negative control. Left image shows a plate culture of E. coli O157:H7 ATCC 43888 infected by the above phages, as labeled. Right image shows a plate culture of Citrobacter sedlakii ATCC 51493 infected by the above phages, as labeled. Bottom left and right sections of each plate are uninfected controls.

FIG. 13 is a schematic illustration of homologous recombination process used to generate synthetic phage CBA120-SPTD1.chiTSP3 from CBA120 by exchanging CBA120 TSP3 with a recombinant TSP (“CBA120-SPTD1 TSP3”) containing N-terminal domain from CBA120 TSP3 and C-terminal domain from SPTD1 TSP3.

FIG. 14 illustrates pairwise alignment, performed on Emboss Needle, of amino acid sequences of CBA120 TSP3 (SEQ ID:11) and SPTD1 TSP3 (SEQ ID:4). The “neck” region is indicated with a box.

FIG. 15 illustrates pairwise alignment, performed on Emboss Needle, of amino acid sequences of CBA120 TSP3 (SEQ ID:11) and recombinant CBA120-SPTD1 TSP3 (SEQ ID:12). The joining site between amino acids 158 and 159 in the “neck” region (indicated with a box) of recombinant CBA120-SPTD1 TSP3 is indicated by an arrow.

FIG. 16 illustrates pairwise alignment, performed on Emboss Needle, of amino acid sequences of SPTD1 TSP3 (SEQ ID:4) and recombinant CBA120-SPTD1 TSP3 (SEQ ID:12). The “neck” region is labelled and indicated with a box.

FIG. 17 is a schematic illustration of the HR.CBA120-SPTD1.chimeric.TSP3 plasmid insert.

FIG. 18 are photographic images of the plate cultures illustrating the properties of synthetic phages CBA120-SPTD1.chiTSP3 (RBP-CBA120-1) and CBA120-SPTD1.chiTSP2 (RBP-CBA120-2) compared to the parent phages. Plate sections are labeled as follows: 1—CBA120; 2—SPTD1; 3—CBA120-SPTD1.chiTSP2 (RBP-CBA120-2); 4—CBA120-SPTD1.chiTSP3 (RBP-CBA120-1) lysate made in Salmonella 19585; 5—CBA120-SPTD1.chiTSP3 (RBP-CBA120-1) lysate made in E. coli 43888; 6—no-phage negative control.

FIG. 19 is a schematic illustration of homologous recombination process used to generate synthetic phage SPTD1-CBA120.chiTSP4 (RBP-SPTD1-1) from SPTD1 by exchanging SPTD1 TSP4 with a recombinant TSP4 (“SPTD1-CBA120 TSP4”) containing N-terminal domain from SPTD1 TSP4 and C-terminal domain from CBA120 TSP4.

FIG. 20 illustrates pairwise alignment, performed on Emboss Needle, of amino acid sequences of CBA120 TSP4 (SEQ ID:13) and SPTD1 TSP4 (SEQ ID:5). The “neck” region (based on published TSP structures) is marked with a box. The arrow indicates the joining site between amino acid 479 of SPTD1 TSP4 and amino acid 480 of CBA120 TSP4.

FIG. 21 is a schematic illustration of the structure of N-terminally truncated CBA120 TSP4 protein.

FIG. 22 is a schematic illustration of the hypothetical structure of recombinant SPTD1 CBA120 TSP4 protein.

FIG. 23 illustrates pairwise alignment, performed on Emboss Needle, of amino acid sequences of SPTD1 TSP4 (SEQ ID:5) and recombinant SPTD1-CBA120 TSP4, which can be also referred to as SPTD1.ChiTSP4 (SEQ ID:14). The joining site between amino acids 479 of SPTD1 TSP4 and amino acid 480 of CBA120 TSP4 is indicated by an arrow. The “neck” region (based on published TSP structures) is indicated by a box.

FIG. 24 illustrates pairwise alignment, performed on Emboss Needle, of amino acid sequences of CBA120 TSP4 (SEQ ID:13) and recombinant SPTD1-CBA120 TSP4, which can be also referred to as SPTD1.ChiTSP4 (SEQ ID:14). The joining site between amino acid 479 of SPTD1 and amino acid 480 of recombinant SPTD1-CBA120-TSP4 is indicated by an arrow. The neck region (based on published TSP structures) is indicated by a box.

FIG. 25 is a schematic illustration of the HR.SPTD1-CBA120chiTSP4 plasmid insert. Upstream homologous recombination region is a part of SPTD1 TSP4 N-terminal region.

FIG. 26 is a photographic image of the plate culture illustrating the results of the plating of synthetic SPTD1-CBA120.chiTSP4 (RBP-SPTD1-1) on E. coli 078 (ECOR70).

FIG. 27 is a schematic illustration of homologous recombination process used to generate synthetic phage SPTD1-CBA120.chiTSP1 (RBP-SPTD1-2) from SPTD1 by exchanging SPTD1 TSP1 with a recombinant TSP1 (“SPTD1-CBA120 TSP1”) containing N-terminal domain from SPTD1 TSP1 and C-terminal domain from CBA120 TSP1.

FIG. 28 illustrates pairwise alignment, performed on Emboss Needle, of amino acid sequences of CBA120 TSP1 (SEQ ID:15) and SPTD1 TSP1 (SEQ ID:2). The arrow indicates the joining site between amino acid 148 of SPTD1 TSP1 and amino acid 152 of CBA120 TSP1. The “neck” region (based on published TSP structures) is indicated by a box.

FIG. 29 is a schematic illustration of the structure of CBA120 TSP1 protein.

FIG. 30 illustrates pairwise alignment, performed on Emboss Needle, of amino acid sequences of SPTD1 TSP1 (SEQ ID:2) and recombinant SPTD1-CBA120 TSP1, which can be also referred to as SPTD1.ChiTSP1 (SEQ ID:7). The joining site between amino acid SPTD1 TSP1 and amino acid 149 of CBA120 TSP1 is marked with an arrow.

FIG. 31 illustrates pairwise alignment, performed on Emboss Needle, of amino acid sequences of CBA120 TSP1 (SEQ ID:15) and recombinant SPTD1-CBA120 TSP1, which can be also referred to as SPTD1.ChiTSP1 (SEQ ID:7). The joining site between amino acid SPTD1 TSP1 and amino acid 149 of CBA120 TSP1 is marked with an arrow.

FIG. 32 is a schematic illustration of HR.SPTD1-CBA120chiTSP1 plasmid insert. Upstream homologous recombination region is SPTD1 TSP1 N-terminal region, which contains the TD1 and TD2 domains.

FIG. 33 is a schematic illustration of the homologous recombination process used to generate synthetic phage SPTD1-CBA120.chiTSP1-Det7.chiTSP2 (RBP-SPTD1-3) from SPTD1-CBA120.chiTSP1 (RBP-SPTD1-2) by exchanging TSP2 of SPTD1-CBA120.chiTSP1 (RBP-SPTD1-2) with a recombinant TSP2 (“SPTD1-Det7 TSP2”) containing N-terminal domain from SPTD1 TSP2 and C-terminal domain from Det7 TSP2.

FIG. 34 illustrates a sequence alignment, performed on Clustal Omega, of amino acid sequences of Det 7 TSP2 (SEQ ID:16), CBA120 TSP2 (SEQ ID:11) and SPTD1 TSP2 (SEQ ID:3). The arrow indicates the joining site between amino acid 252 of SPTD1 TSP2 and amino acid 256 of Det7 TSP2. The “neck” region based on published TSP structures is indicated by a box.

FIG. 35 is a schematic illustration of the structure of CBA120 TSP2 protein.

FIG. 36 is a schematic illustration of the predicted monomeric structure of a recombinant TSP containing N-terminus of SPTD1 TSP2 and C-terminus of Det7 TSP2 (SPTD1-Det7 TSP2 or SPTD1.Chi.TSP2).

FIG. 37 illustrates pairwise alignment, performed on Emboss Needle, of amino acid sequences of recombinant SPTD1-Det7 TSP2, which can also be referred to as SPTD1.Chi.TSP2 (SEQ ID:8). and SPTD1 TSP2 (SEQ ID:3). The arrow indicates the joining site between amino acid 252 of SPTD1 TSP2 and amino acid 256 of Det7 TSP2.

FIG. 38 illustrates pairwise alignment, performed on Emboss Needle, of amino acid sequences of recombinant SPTD1-Det7 TSP2 (SEQ ID:8) and Det7 TSP2 (SEQ ID:16). The arrow indicates the joining site between amino acid 252 of SPTD1 TSP2 and amino acid 256 of Det7 TSP2.

FIG. 39 is a schematic illustration of HR.SPTD1-CBA120chiTSP2 plasmid insert. Upstream homologous recombination region is SPTD1 TSP2 N-terminal region, which contains attachment domain (AD), XD2, XD3, and TD1 domains.

FIG. 40 is a schematic illustration of the homologous recombination process used to generate synthetic phage RBP-SPTD1-5 from SPTD1-CBA120.chiTSP1-Det7.chiTSP2 (RBP-SPTD1-3) by exchanging TSP4 of SPTD1-CBA120.chiTSP1-Det7.chiTSP2 (RBP-SPTD1-3) with a recombinant TSP4 (“SPTD1.TSP4-TR2.TSP”) containing N-terminal domain from SPTD1 TSP4 and C-terminal domain from TR2 TSP.

FIG. 41 illustrates pairwise alignment, performed on Emboss Needle, of amino acid sequences of SPTD1 TSP4 (SEQ ID:5) and TR2 TSP (SEQ ID:18). The joining site between amino acids 479 of SPTD1 TSP 4 and amino acid 363 of TR2 TSP is indicated by an arrow. The “neck” regions (based on AlphaFold2 structural predictions and published homology) are indicated by a box.

FIG. 42 is a schematic illustration of the predicted structure generated by AlphaFold2 of SPTD1 TSP4.

FIG. 43 is a schematic illustration of the predicted structure generated by AlphaFold2 of TR2 TSP.

FIG. 44 is a schematic illustration of the predicted structure generated by AlphaFold2 of a recombinant TSP containing N-terminus of SPTD1 TSP4 and C-terminus of TR2 TSP. Protein structure of chimeric TSP4

FIG. 45 illustrates pairwise alignment, performed on Emboss Needle, of amino acid sequences of SPTD1 TSP4 (SEQ ID:5) and a recombinant TSP containing N-terminal domain of SPTD1 TSP4 and C-terminal domain of TR2 TSP, which can be referred to as “SPTD1.TSP4-TR2.TSP” or “chi.TSP4” (SEQ ID:18). The joining site between amino acids 479 of SPTD1 TSP 4 and amino acid 363 of TR2 TSP is indicated by an arrow. The “neck” regions (based on AlphaFold2 structural predictions and published homology) are indicated by a box.

FIG. 46 illustrates pairwise alignment, performed on Emboss Needle, of amino acid sequences of TR2 TSP (SEQ ID:17) and a recombinant TSP containing N-terminal domain of SPTD1 TSP4 and C-terminal domain of TR2 TSP, which can be referred to as “SPTD1.TSP4-TR2.TSP” or “chi.TSP4” (SEQ ID:18). The joining site between amino acids 479 of SPTD1 TSP 4 and amino acid 363 of TR2 TSP is indicated by an arrow. The “neck” regions (based on AlphaFold2 structural predictions and published homology) are indicated by a box.

FIG. 47 is a schematic illustration of the HR.SPTD1.TSP4-TR2.TSP plasmid insert. Upstream homologous recombination region is SPTD1 TSP4 N-terminal region.

FIG. 48 is a photographic image of the plate culture illustrating the results of the plating of synthetic bacteriophage RBP-SPTD1-5 on Salmonella Kentucky

### DETAILED DESCRIPTION

### Overview

Described in the present disclosure are compositions, methods, systems, and kits related to synthetic phages with a customized host range and recombinant phage tail-spike proteins (TSPs) with customized ability to recognize microorganisms. Customization of the host range of a synthetic phage according to the present disclosure may involve conferring on the synthetic phage an ability to recognize one or more target host, and/or eliminating recognition of one or more hosts from a synthetic phage. The ability of the synthetic phage to recognize or not recognize one or more hosts may be referred to in the present disclosure as “host range,” “host recognition capacity,” or “host recognition ability.” In some embodiment, customized host range of a synthetic phage according to the present disclosure may be altered in comparison to a natural or recombinant parent phage from which the synthetic phage is constructed. In some other embodiments, a synthetic phage with a customized host range may be constructed de novo using the principles and the techniques described in the present disclosure along with the general principles of synthetic biology, genetic engineering, and other relevant disciplines. Exemplary synthetic phages are constructed from or based on Ackermannviridae phages. Customization of an ability of a recombinant TSP to recognize a microorganism according to the present disclosure may involve altering the ability of the TSP to bind to a surface receptor of a microorganism, which may involve altering a TSP domain (for example, C-terminal domain) responsible for binding to the surface receptor. In some embodiment, an ability of a recombinant TSP to recognize a microorganism may be altered in comparison to a natural or recombinant parent TSP from which the recombinant TSP is constructed. In some other embodiments, a recombinant TSP may be constructed de novo using the principles and the techniques described in the present disclosure along with the general principles of synthetic biology, genetic engineering, and other relevant disciplines. Exemplary recombinant TSPs are constructed from or based on TSPs derived from Ackermannviridae phages.

As envisioned by the inventors and described in the present disclosure, in some embodiments, a customized host range is imparted on a synthetic phage, which may be constructed from or based on an Ackermannviridae phage, by one or more recombinant TSPs. Some embodiments of synthetic phages incorporate one or more recombinant TSPs that include a combination of the N-terminal region and the C-terminal region that is engineered in a laboratory. As discussed in more detail further in this disclosure, a C-terminal region of a TSP is responsible for recognizing a target host. Accordingly, in one example, by including into a recombinant TSP of a synthetic phage a C-terminal region that is capable of recognizing a target host, it is possible to confer on a synthetic phage according to the embodiments of the present invention an ability to recognize the target host. Other examples and embodiments are envisioned and are described in more detail further in this disclosure, along with the methods of constructing synthetic phages according to the embodiments of the present invention and other related methods, methods of using synthetic phages according to the embodiments of the present invention, as well as systems and kits related to the synthetic phages according to the embodiments of the present invention.

As conceived and envisioned by the inventors, embodiments of the present invention, examples of which are described in the present disclosure, possess various advantages. For example, embodiments of the present invention provide versatile, highly customizable and easy-to-use platform for producing synthetic phages with a customized host range, as well as recombinant TSPs with customized ability to recognize microorganisms. For example, based on the embodiments of the present invention, different plasmids encoding for various recombinant TSPs with various abilities to recognize microorganism may be produced and subsequently used to engineer, with high efficiency (reduced time and costs) synthetic phages with desired customized host ranges. In another example, phages specific for highly pathogenic bacteria, such as Burkholderia pseudomallei or Yersinia pestis, may be engineered to also infect a nonpathogenic host, allowing phage production to be conducted at lower biosafety levels with reduced risk to personnel and associated costs. In another example, embodiments of the present invention may allow production of synthetic phages capable of infecting various bacteria without the need to perform phage production in phage hosts that are challenging to culture or possess antibiotic resistance.

In some exemplary embodiments, Ackermannviridae phage TSPs are used to construct recombinant TSPs. TSPs are structurally similar between different Ackermannviridae sub-categories, which allows using recombinant TSPs to engineer synthetic phages according to the embodiments from the present invention from a wide range of Ackermannviridae phages. The methods of constructing synthetic phages envisioned by the inventors may use TSP amino acid sequences from published databases, which reduces or eliminates the need for a laboratory experimentation to identify phages with desired properties for constructing recombinant TSPs and/or synthetic phages with customized host ranges according to the embodiments of the present invention. There is evidence that horizontal gene transfer of TSP-encoding nucleic acid sequences occurs between Ackermannviridae and non-Ackermannviridae phages, such as with podoviruses (bacteriophages of Podoviridae family). For example, podovirus Bacteriophage P22 TSP appears to be related to Ackermannviridae Salmonella phage Det7 TSP. Thus, it is envisioned that amino acid sequences derived from TSPs or short-tail fiber protein from non-Ackermannviridae phages, such as, but not limited to, Podoviridae or Siphoviridae, may be used in recombinant TSPs and/or synthetic phages with customized host ranges according to the embodiments of the present invention.

### Terms and Concepts

A number of terms and concepts are discussed below. They are intended to facilitate the understanding of various embodiments of the invention in conjunction with the rest of the present document and the accompanying figures. These terms and concepts may be further clarified and understood based on the accepted conventions in the fields of the present invention, as well as the description provided throughout the present document and/or the accompanying figures. Some other terms can be explicitly or implicitly defined in other sections of this document and in the accompanying figures, and may be used and understood based on the accepted conventions in the fields of the present invention, the description provided throughout the present document and/or the accompanying figures. The terms not explicitly defined can also be defined and understood based on the accepted conventions in the fields of the present invention and interpreted in the context of the present document and/or the accompanying figures.

Unless otherwise dictated by context, singular terms shall include pluralities, and plural terms shall include the singular. Generally, nomenclatures used in connection with, and techniques of, cell and tissue culture, molecular biology, immunology, microbiology, genetics and protein and nucleic acid chemistry are those well-known and commonly used. Known methods and techniques are generally performed according to conventional methods well-known and as described in various general and more specific references, unless otherwise indicated. The nomenclatures used in connection with the laboratory procedures and techniques described in the present disclosure are those well-known and commonly used.

Unless otherwise specified and/or dictated by context, nomenclatures used in connection with, and techniques of, cell and tissue culture, molecular biology, immunology, microbiology, genetics and protein and nucleic acid chemistry and hybridization described herein are those well-known and commonly used in the art. Known methods and techniques are generally performed according to conventional methods well-known in the art and as described in various general and more specific references that are discussed throughout the present specification unless otherwise indicated. Enzymatic reactions and purification techniques are performed according to manufacturer's specifications, as commonly accomplished in the art or as described herein. The nomenclatures used in connection with the laboratory procedures and techniques described herein are those well-known and commonly used in the art.

As used in the present disclosure, the terms “a”, “an”, and “the” can refer to one or more unless specifically noted otherwise.

The use of the term “or” is used to mean “and/or,” unless explicitly indicated to refer to alternatives only, or the alternatives are mutually exclusive, although the disclosure supports a definition that refers to only alternatives and “and/or.” As used in the present disclosure “another” can mean at least a second or more.

As used in the present disclosure, and unless otherwise indicated, the terms “include,” “including,” and, in some instances, similar terms (such as “have” or “having”) mean “comprising.”

When a numerical range is provided in the present disclosure, the numerical range includes the range endpoints unless otherwise indicated. Unless otherwise indicated, numerical ranges include all values and subranges tin the present disclosure, as if explicitly written out.

The terms “about” and “approximately,” as used in the present disclosure, shall generally mean an acceptable degree of error for the quantity measured, given the nature or precision of the measurements. Exemplary degrees of error are within 20%, 10%, 9%, 8%, 7%, 6%, 5%, 4%, 3%, 2%, or 1% of a given value or range of values. For example, any reference to “about X” or “approximately X” specifically indicates at least the values X, 0.9×, 0.91×, 0.92×, 0.93×, 0.94×, 0.95×, 0.96×, 0.97×, 0.98×, 0.99×, 1.01×, 1.02×, 1.03×, 1.04×, 1.05×, 1.06×, 1.07×, 1.08×, 1.09×, and 1.10×. In another example, the terms “about” or “approximately” in relation to a reference numerical value can include a range of values plus or minus 10%, 9%, 8%, 7%, 6%, 5%, 4%, 3%, 2%, or 1% from that value. Thus, expressions “about X” or “approximately X” are intended to describe a claim limitation of, for example, “0.98×.” Numerical quantities given in the present disclosure are approximate unless stated otherwise, meaning that the term “about” or “approximately” can be inferred when not expressly stated. When the terms “about” or “approximately” are applied to the beginning of a numerical range, they apply to both ends of the range. Where a series of values is prefaced with the terms “about” or “approximately,” these terms are intended to modify each value included in the series.

The terms “administering” or “administration,” when using in the context of administration of a composition described in the present disclosure to a subject (and the related terms and expression), refer to the act of physically delivering a substance as it exists outside the body (for example, a composition comprising one or more synthetic phages described in the present disclosure) into a subject. Administration can be by mucosal, intradermal, intravenous, intramuscular, subcutaneous delivery and/or by any other known methods of physical delivery. Administration encompasses direct administration, such as administration to a subject by a medical professional or self-administration, or indirect administration, which may be the act of prescribing a composition described in the present disclosure.

As used herein, an “analyte” refers to a molecule, compound or cell that is being measured. The analyte of interest may, in certain embodiments, interact with a binding agent. As described herein, the term “analyte” may refer to a protein or peptide of interest. An analyte may be an agonist, an antagonist, or a modulator. Or, an analyte may not have a biological effect. Analytes may include small molecules, sugars, oligosaccharides, lipids, peptides, peptidomimetics, organic compounds and the like.

As used herein, the term “chimeric” and related terms and expressions, refer to nucleic acid sequences, amino acid sequences, polypeptides, or proteins that are artificially constructed by combining or bringing together, respectively, nucleic acid sequences, amino acid sequences, or polypeptide or protein regions or domains from two or more different sources (such as phages). The resulting combinations are not found in nature. For example, combining the N-terminal region of a TSP from one phage with the C-terminal region of a TSP from a different phage. In other cases, different locations may refer to different proteins within the same phage. For example, replacing the C-terminal region of TSP1 in CBA120 with the C-terminal region of TSP2 in CBA120, thereby artificially duplicating the TSP2 C-terminal region.

The term “detectable moiety” or “detectable biomolecule” or “reporter” or “indicator” or “indicator moiety” refers to a molecule or a compound produced by a molecule (such as an enzyme) that can be measured in a quantitative assay. For example, an indicator or indicator moiety may comprise an enzyme that may be used to convert a substrate to a product that can be measured. An indicator or indicator moiety may be an enzyme that catalyzes a reaction that generates bioluminescent emissions (for example, luciferase). Or, an indicator or indicator moiety may be a radioisotope that can be quantified. Or, an indicator moiety may be a fluorophore. Or, other detectable molecules may be used. The term “indicator gene” is used to refer to a gene encoding an indicator, such as a protein, for example, an enzyme.

The term “host” and the related terms and expressions are used in the present disclosure in reference to phages to denote microorganisms that a phage is capable of infecting. Phages can lyse host cells and release new virions upon lysis, transfer genes between hosts, and form lysogens, which can modify host function. As used herein, the expression “host range” and the related terms and expression refer to a range or number of hosts infected by a phage. In other words, “host range” describes the breadth of organisms (genera, species, strains, or other taxa) a phage is capable of infecting, with limits on host range stemming from phage, host, or environmental characteristics. Host range of some phages is rather narrow, only having the ability to infect a few strains within the same species. Other phages can infect many species of hosts, sometimes across different genera. The breadth of a host range of particular phage may be due in part to the specificity of phages' host binding proteins, biochemical interactions during infection, presence of related prophages or particular plasmids, and host phage-resistance mechanisms. The expressions “customized host range,” “altered host range,” “modified host range,” “altered tropism” and related expressions are used herein to refer to phage host range that is artificially changed. Such host change ranges can be effected by at least some of the methods described in the present disclosure, as well as other methods.

The terms “nucleic acid,” “nucleic acid sequence,” “nucleotide sequence,” “oligonucleotide,” “polynucleotide” and the related terms and expressions refer to deoxyribonucleic acids (DNA) or ribonucleic acids (RNA) and their polymers. Nucleic acid sequences, as discussed in the present disclosure, encompass all forms of nucleic acids, including, but not limited to, single-stranded forms, double-stranded forms, hairpins, stem-and-loop structures, and the like. When an RNA sequence is described, its corresponding DNA sequence is also described, wherein uridine is represented as thymidine. When a DNA sequence is described, its corresponding RNA sequence is also described, wherein thymidine is represented as uridine. Unless specifically limited, the term “nucleic acid” and the related terms and expressions encompass nucleic acids containing known analogues of natural nucleotides that have similar properties as the reference nucleic acid, and are metabolized in a manner similar to naturally occurring nucleotides. A nucleic acid sequence can include combinations of deoxyribonucleic acids and ribonucleic acids. Such deoxyribonucleic acids and ribonucleic acids include both naturally occurring molecules and synthetic analogues. Unless otherwise indicated, a particular nucleic acid sequence also implicitly encompasses degenerate codon substitutions, alleles, orthologs, SNPs, and complementary sequences as well as the sequence explicitly indicated. Degenerate codon substitutions may be achieved by generating sequences in which the third position of one or more selected (or all) codons is substituted with mixed-base and/or deoxyinosine residues.

The terms “oligonucleotide,” “polynucleotide” or “nucleic acid” encompass DNA or RNA molecules, including the molecules produced synthetically or by recombinant technology. Oligonucleotides, polynucleotides or nucleic acids may be single-stranded or double-stranded.

As used herein, “phage” includes one or more of a plurality of viruses that can invade living bacteria, fungi, mycoplasma, protozoa, yeasts, and other microscopic living organisms. In this disclosure, the term “phage” and the related terms include viruses such as bacteriophages, which can invade bacteria, viruses infecting archaea, which can be referred to as archaeal phages or bacteriophages, mycobacteriophages, which can invade mycobacteria (a family bacteria, which includes the mycobacteria of Mycobacterium tuberculosis complex, including the causative agents of tuberculosis, and the mycobacteria of Mycobacterium avis complex, including the causative agents of tuberculosis), mycoviruses, which can invade fungi, mycoplasma phages, as well as the viruses that may infect protozoa, yeasts, and other microscopic living organisms. Here, “microscopic” means that the largest dimension is one millimeter or less. Phages are viruses that have evolved in nature to use microscopic organisms as a means of replicating themselves. In nature, a phage attaches itself to a microorganism and injects its DNA (or RNA) into that microorganism, and then can induce the microorganism to replicate the phage hundreds or even thousands of times. This is referred to as phage amplification.

The terms “peptide,” “polypeptide” or “protein” are used to refer polymer of amino acids linked by native amide bonds and/or non-native amide bonds. Peptides, polypeptides or proteins may include moieties other than amino acids (for example, lipids or sugars). Peptides, polypeptides or proteins may be produced synthetically or by recombinant technology. The amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V). In the broadest sense, the naturally occurring amino acids can be divided into groups based upon the chemical characteristic of the side chain of the respective amino acids. By “hydrophobic” amino acid is meant either His, Leu, Met, Phe, Trp, Tyr, Val, Ala, Cys or Pro. By “hydrophilic” amino acid is meant either Gly, Asn, Gln, Ser, Thr, Asp, Glu, Lys, Arg or His. This grouping of amino acids can be further sub-classed as follows: by “uncharged hydrophilic” amino acid is meant either Ser, Thr, Asn or Gln. By “acidic” amino acid is meant either Glu or Asp. By “basic” amino acid is meant either Lys, Arg or His. Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (a carbon that is bonded to a hydrogen, a carboxyl group, an amino group) but have modified side-chain groups or modified peptide backbones, such as homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid. Amino acids may be referred to by either the commonly known three letter symbols or by the one-letter symbols recommended by the IUPAC-IUB Biochemical Nomenclature Commission. A “domain” of a protein or a polypeptide refers to a region of the protein or polypeptide defined by structural and/or functional properties. Exemplary function properties include enzymatic activity and/or the ability to bind to or be bound by another protein or non-protein entity.

As used herein, the term “recombinant” and related terms and expressions refer to genetic (that is, nucleic acid) modifications as usually performed in a laboratory to bring together genetic material that would not otherwise be found. This term can be used interchangeably with the term “modified,” “synthetic,” “engineered,” “constructed,” etc.

The term “solid support” or “support” means a structure that provides a substrate and/or surface onto which biomolecules, cells, etc. may be bound. For example, a solid support may be an assay well (that is, such as a microtiter plate or multi-well plate), or the solid support may be a location on a filter, an array, or a mobile support, such as a bead or a membrane (for example, a filter plate or lateral flow strip).

The terms “individual”, “subject”, and “patient” can be used interchangeably in the present disclosure to refer to a non-human animal or a human. Examples of subjects include, but are not limited to: humans and other primates, including non-human primates, such as chimpanzees and other apes and monkey species; farm animals, such as cattle, sheep, pigs, goats and horses; domestic mammals such as dogs and cats; laboratory animals including rodents, such as mice, rats and guinea pigs; birds, including domestic, wild and game birds, such as chickens, turkeys and other gallinaceous birds, ducks, geese, and the like. The terms individual, subject, and patient, by themselves, do not denote a particular age, sex, race, or clinical status. Thus, subjects of any age, whether male or female, are intended to be covered by the present disclosure and include, but are not limited to the elderly, adults, children, babies, infants, and toddlers. Likewise, the methods of the present invention can be applied to any human race, including, for example, Caucasian (white), African American (black), Native American, Native Hawaiian, Hispanic, Latino, Asian, and European. An infected subject is a subject that is known to have been infected by an infectious organism, such as a bacterium.

The term “sample” and the related terms and expressions encompass, but are not limited to, an environmental sample, a food sample, a water sample, a medical sample, a clinical sample, or a veterinary sample. Samples may be liquid, solid, or semi-solid. Samples may be swabs of solid surfaces. Samples may include environmental materials, such as water samples, or the filters from air samples, or aerosol samples from cyclone collectors. Samples may be samples of fish, meat, such as beef, pork or lamb, poultry, processed foods, peanut butter, powdered infant formula, powdered milk, teas, starches, eggs, milk, cheese, or other dairy products. Medical, clinical or veterinary samples include, but are not limited to, blood, sputum, cerebrospinal fluid, urine and fecal samples. In some embodiments, samples may be different types of swabs. The term “sample” encompasses various appropriate control samples. For example, in the context of microorganism detection control samples without microorganisms of interest may be assayed as controls for background signal levels.

The term “variant,” when used in the present disclosure in reference amino acid or nucleic acid sequences encompasses homologues, variants, isoforms, fragments, mutants, modified forms and other variations of the amino acid or nucleic acid sequences described in this document. The term “homologous,” “homologues” and other related terms used in this document in reference to various amino acid, are intended to describe a degree of sequence similarity among amino acid sequences, calculated according to an accepted procedure. Homologous or similar sequences (which may also be described as “variants”) may be at least 60%, 65%, 70%, 75%, 80%, 85%, 90%, 91%, 92%, 93%, 94%, 95%, 96%, 97%, 98% 99% or 100% homologous (or also described as having at least 60%, 65%, 70%, 75%, 80%, 85%, 90%, 91%, 92%, 93%, 94%, 95%, 96%, 97%, 98% 99% or 100% “sequence identity” or “sequence similarity.” As used herein, “percent homology” (or “sequence identity,” or “sequence similarity”) of two amino acid sequences is determined using the algorithm of Karlin and Altschul, which is incorporated into Basic Local Alignment Search Tool (BLAST®, National Library of Medicine, Bethesda, Maryland) programs, available for public use through the website of the National Institutes of Health (U.S.A.). The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (Henikoff and Henikoff, 1989). The BLAST algorithm also performs a statistical analysis of the similarity between two sequences (Karlin and Altschul, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20. variants or isoforms may be employed alternatively to or in conjunction with homology. To obtain gapped alignments for comparison purposes, Gapped BLAST is utilized. When utilizing BLAST and Gapped BLAST programs, the default parameters of the respective programs (for example, XBLAST and NBLAST) are used. Percent “homology” or “sequence similarity” may be used in the present disclosure to describe fragments, variants or isoforms of amino acids or nucleic acid sequences, but other ways of describing fragments. Percent “homology” or “sequence similarity” may also be used in the present disclosure to describe nucleic acid sequences and amino acid sequences that are “derived from” and “based on” other sequences. For example, if an amino acid sequence of a recombinant TSP, a region of a TSP (such as an N-terminal region, a C-terminal region, or any other region) according to the present disclosure is said to be “derived from” or “based on” another TSP amino acid sequence (which may be described as “parent amino acid sequence” or “source amino acid sequence”), the amino acid sequence of the recombinant TSP may have at least 60%, 65%, 70%, 75%, 80%, 85%, 90%, 91%, 92%, 93%, 94%, 95%, 96%, 97%, 98% 99% or 100% “sequence identity” or “sequence similarity” to the source amino acid sequence.

The expression “conservatively modified variant” and related expression may apply to amino acid sequences, as well to nucleic acid sequences encoding amino acid sequence. Substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

“Virus” and the related terms and expressions are used in both the plural and singular senses. “Virion” refers to a single virus. For example, the expression “phage virion” refers to a phage particle.

### Phages

Phages that possess tails—molecular machines that specifically recognize and attach to host cells, penetrate cell envelope and deliver phage nucleic acids into the cells—can be referred to as “tailed phages.” Among naturally occurring bacteriophages, tailed phages are typically classified into the class Caudoviricetes. In addition to taxonomic separation by genomic relatedness, tailed phages can and have been historically distinguished by morphology. Myoviruses are characterized by long contractile tails (ex. phages of families Straboviridae, Kyanoviridae, Herelleviridae, Chaseviridae, and Ackermannviridae). Siphoviruses are characterized by long non-contractile tails (ex. phages of families Demerecviridae and Drexlerviridae. Podoviruses are characterized by short non-contractile tails (ex. phages of family Autographiviridae). Myoviruses of the family Ackermannviridae have a non-enveloped head, a neck without a collar, a small base plate, and a contractile tail. Host range of tailed phages is determined by the structures they use to bind to host cells. These structures include tail fibers, tail spikes, and tail tips. Host-cell binding structures of tailed phages bind to surface receptors of a host cell. Infection of a host cell by a tailed phage virion starts with reversible recognition of host cell surface receptor by a binding structure, followed by irreversible binding, and delivery of phage nucleic acids (DNA in case of Caudoviricetes) into the host cell. Tail fibers, tail spikes, and tail tips act as receptor-binding proteins (RBPs) and specifically recognize host cell surface receptors. Cell surface polysaccharides, including lipopolysaccharides, of a host cell often serve as surface receptors for phages. Some other cell known cell surface receptors recognized by phages are teichoic acid, porins, and cell appendages, such as flagella, and pili. More than one cell surface receptor may be involved in recognition of a host cell by a phage virion. Tailed phages use the tail and associated RBPs to interact with the host cells and to create a channel for delivery of phage nucleic acids into the host cell. High genetic plasticity of the RBPs of tailed phages allows the tailed phages to adapt their host range. Phage taxonomy, structure of tailed phages, including those of family Ackermannviridae, as well as the mechanisms of recognition of host cells used by tailed phages, are discussed, for example, in one or more of Nobrega et al. (2018), Prokhorov et al. (2017), Sorensen et al. (2021), Greenfield et al. (2020), Plattner et al. (2019), Bertozzi Silva et al. (2016), and Ackermann (2011). It is to be understood that phage taxonomy and classification are changing areas. For example, current official phage classification has a number of “floating” members, which may lack “order” and/or “family” designation. Many current phage designations are based on morphology, but not necessarily on molecular taxonomy.

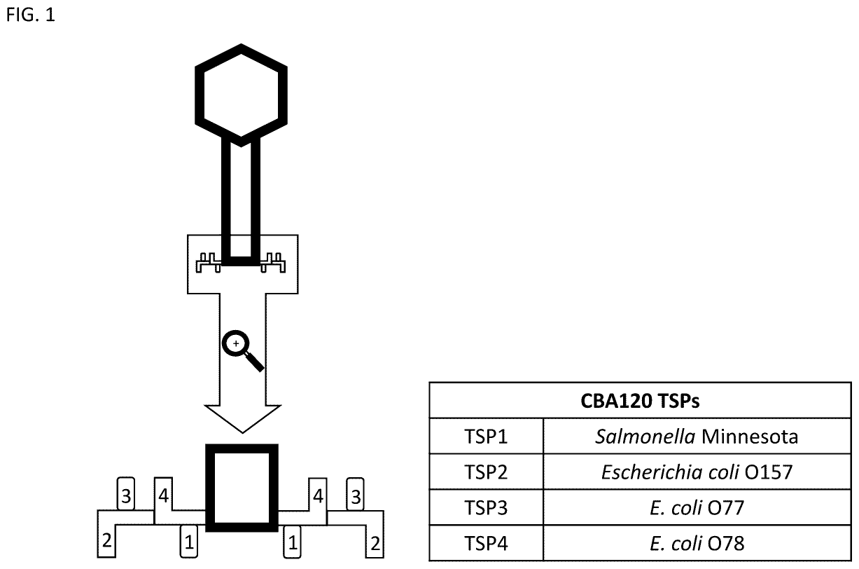

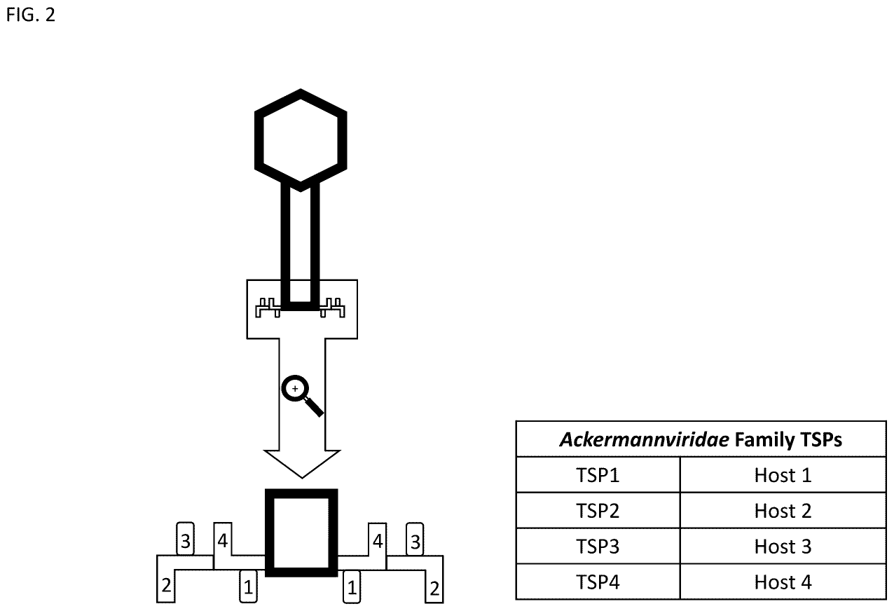

The tail of phages of family Ackermannviridae includes up to four tail-spike proteins (TSPs), each recognizing a specific host. Some phages of Ackermannviridae family express four different TSPs, but some contain three TSP genes. Some recent studies of TSP sequence diversity classify Ackermannviridae TSPs into distinctive subtypes of TSP1, TSP2, TSP3 and TSP4, and find each TSP subtype to be specifically associated with the genera (for example, Kuttervirus, Agtrevirus, Limestonevirus, Taipeivirus) within Ackermannviridae family. See, for example, Sorensen et al. (2021). While amino acid sequences of Ackermannviridae TSPs are diverse, overall TSP structure is well-conserved. Ackermannviridae TSP are stable homotrimers, in which each monomer displays right-handed β-helices. The assembly of all the TSPs on an Ackermannviridae phage virion forms a branched structure attached to the baseplate. FIG. 1 schematically illustrates four TSPs of CBA120, a member of Ackermannviridae family. Each of TSPs 1-4 recognizes a unique bacterial serotype (TSP1 —Salmonella enterica subsp. enterica serovar Minnesota; TSP2-E. coli 0157; TSP3 —E. coli 077; TSP4 —E. coli 078). FIG. 2 is a schematic illustration of TSPs for an exemplary phage in the Ackermannviridae family with four TSPs. Each TSP is capable of recognizing a unique host cell. The TSPs form a branched complex attached to the baseplate of a phage virion. Described in the present disclosure and included among the embodiments of the present invention are recombinant TSPs, some of which are derived from TSPs from Ackermannviridae phages. It is to be understood, however, that recombinant TSPs according to the embodiments of the present invention are not limited to those derived from Ackermannviridae phages. Recombinant TSPs according to the embodiments of the present invention may be derived, at least in part, from TSPs or homologous structures often identified as short tail fibers or simply tail fibers found in other phage families, such as, but not limited to, Autographiviridae, Straboviridae, Herelleviridae, Chaseviridae, Demerecvridae, and Drexlerviridae. Short-tailed fibers of many tailed phages may contain amino-acid sequences useful for as source sequences for C-terminal region sequences of recombinant TSPs according to the present. Amino acid sequences of recombinant TSPs according to the embodiments of the present invention may be derived, at least in part, from amino acid sequences of various tailed phages (Cauodovriricetes). Recombinant TSPs according to the embodiments of the present invention can also be designed and constructed de novo.

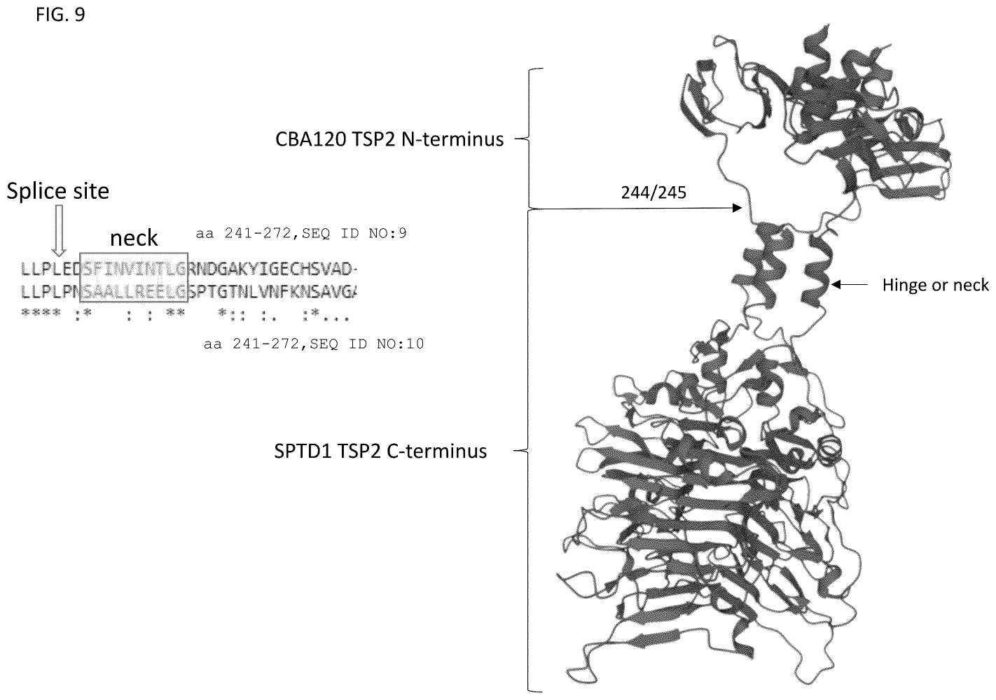

A TSP homotrimer includes an N-terminal region or domain, an elongated C-terminal region or domain, and a helical “neck” or “hinge” region connecting the N-terminal and the C-terminal regions or domains. A structure of a TSP trimer is illustrated in FIG. 9 which schematically depicts a recombinant TSP according to an embodiment described in the present disclosure. Prior to the characterization of TSPs, many genes that likely encode TSPs have been identified instead as tail fiber and short tail fiber proteins. Many Ackermannviridae viruses thus have their TSPs mislabeled as tail fiber proteins. The N-terminal domain or region of a TSP (which can be referred to as “head-binding domain, “head domain” and other related terms and expression) is structurally important in facilitating either binding to the baseplate of a phage virion or binding to other TSPs to form a branched tail-spike complex. A TSP serves as an RBP that allows for initial reversible contact between a phage virion and a host cell. The C-terminal domain or region of a TSP (which can be referred to as a “receptor-binding domain,” “receptor-binding region,” “body domain” and other related terms and expressions) contains a receptor-binding/catalytic module that facilitates binding and degradation of the receptor of the host cell. After binding to a surface receptor on a host cell, the enzymatic activity of the TSP leads to irreversible binding of phage virion to its host cell. For example, a C-terminal domain of a TSP can exhibit lyase, glycosidase, or esterase activity towards a lipopolysaccharide (LPS) of a host cell, which leads to irreversible binding of a phage virion to its host cell. TSPs do not lyse host cells, rather, it is thought that, by cleaving or modifying host cell surface receptors, they facilitate subsequent cell wall penetration.

Described in the present disclosure and included among the embodiments of the present invention are synthetic phages, some of which are derived from naturally occurring Ackermannviridae phages (although synthetic phages according to the embodiments of these invention are not limited to synthetic Ackermannviridae-derived phages, and may be derived from or based on other tailed phages or designed and synthesized de novo). Known Ackemannviridae taxa include: Aglimvirinae, which, in turn, include Agtreviruses (for example, Agtrevirus AG3, Agtrevirus MK13, Agtrevirus P46FS4, and Agtrevirus SKML39), Limestoneviruses (for example, Limestonevirus limestone and Limestonevirus RC2014) as well as a unclassified Aglimvirinae (for example, Dickeya phage phiDP10.3, Dickeya phage phiDP23.1, Enterobacter phage fGh-Ec102, Escherichia phage PC3, Escherichia phage PH4, Escherichia phage vB_EcoM-RPN242, and Escherichia phage vB_EcoM-ZQ1); Campanilevirus (for example, Vibrio phage YC and Vibrio phage vB_VcorM_GR28A); Cvivirinae, which, in turn, include Kutterviruses (such as Kuttervirus aagejoakim, Kuttervirus allotria, Kuttervirus barely, Kuttervirus bering, Kuttervirus BSP101, Kuttervirus CBA120, Kuttervirus Det7, Kuttervirus dinky, Kuttervirus ECML4, Kuttervirus EP75, Kuttervirus FEC14, Kuttervirus GG32, Kuttervirus heyday, Kuttervirus kv38, Kuttervirus maane, Kuttervirus marshall, Kuttervirus maynard, Kuttervirus moki, Kuttervirus mutine, Kuttervirus pertopsoe, Kuttervirus PhaxI, Kuttervirus PM10, Kuttervirus rabagast, Kuttervirus S118, Kuttervirus SE14, Kuttervirus SeB, Kuttervirus SeG, Kuttervirus SeJ, Kuttervirus SenASZ3, Kuttervirus SeSzl, Kuttervirus SFP10, Kuttervirus SH19, Kuttervirus SJ2, Kuttervirus SJ3, Kuttervirus SP1, Kuttervirus SS9, Kuttervirus STML131, Kuttervirus STW77, and Kuttervirus ViI); Kujavirus, which include Kujavirus kuja (exemplified by Vibrio phage vB_VchM_Kuja); Miltonvirus, which include Miltonvirus 3M (exemplified by Serratia phage vB_SmaA_3M), Miltonvirus MAM1 (exemplified by Serratia phage 2050H1 and Serratia phage phiMAM1), and unclassified Miltonvirus (exemplified by Serratia phage KNP4); Nezavisimistyvirus, which include Nezavisimistyvirus buel (exemplified by Erwinia phage vB_EamM-Buel) and Nezavisimistyvirus Ea2809 (exemplified by Erwinia phage phiEa2809); Taipeivirus, which include Taipeivirus 0507KN21, Taipeivirus IME250 (exemplified by Serratia phage vB-Sru-IME250), Taipeivirus KpS110 (exemplified by Klebsiella phage vB_KpnM_KpS110), Taipeivirus KWBSE436 (exemplified by Escherichia phage vB_EcoM_KWBSE43-6), Taipeivirus magnus (exemplified by Klebsiella phage Magnus), Taipeivirus may (exemplified by Klebsiella phage May), Taipeivirus menlow (exemplified by Klebsiella phage Menlow), Taipeivirus UPM2146 (exemplified by Klebsiella phage UPM 2146), and unclassified Taipeivirus, (such as Klebsiella phage K751, Klebsiella phage PWKp5, Klebsiella phage T751, Klebsiella phage T765, Klebsiella phage vB_KqM-Bilbo, Klebsiella phage vB_KqM-LilBean, Klebsiella phage vB_KqM-Westerburg, Klebsiella virus UPM 2146); Tedavirus, which include Tedavirus A829 (exemplified by Aeromonas phage phiA8-29); Vapseptimavirus, which include Vapseptimavirus VAP7 (exemplified by Vibrio phage BX-1, Vibrio phage VAP7, and Vibrio phage VP-1). Ackermannviridae also include unclassified Ackermannviridae, such as Acinetobacter phage SH-Ab 15599, Agrobacterium phage Atu_ph04, Agrobacterium phage OLIVR5, Agrobacterium phage OLIVR6, Ralstonia phage RSP15, Rhizobium phage AF3, Rhizobium phage P9VFCI, Rhizobium phage RHph_II_18, Rhizobium phage RHph_II_9, Rhizobium phage RHph_I34, Rhizobium phage RHph_I46, Rhizobium phage RHph_I9, Rhizobium phage RHph_N34, Rhizobium phage RHph_Y68, Rhizobium phage RL2RES, Rhizobium phage RL38J1, Rhizobium phage vB_RleM_P10VF, Salmonella phage BRM 13312, Salmonella phage BRM 13314, Sinorhizobium phage phiM9, Stenotrophomonas phage vB_SmaS-DLP_6, Vibrio phage 1.244.A._10N.261.54.C3, Vibrio phage 1.255.0._10N.286.45.F1, Vibrio phage 144E46.1, Vibrio phage 207E48.1, Ackermannviridae sp. ctaCq7, Ackermannviridae sp. ctClB2, Ackermannviridae sp. ctFRM8, Ackermannviridae sp. ctjwt21, Ackermannviridae sp. ctkHJ36, Ackermannviridae sp. ctQad106, and Ackermannviridae sp. ctUml7.

### Synthetic Phages and Recombinant Tail Spike Proteins (TSPs)

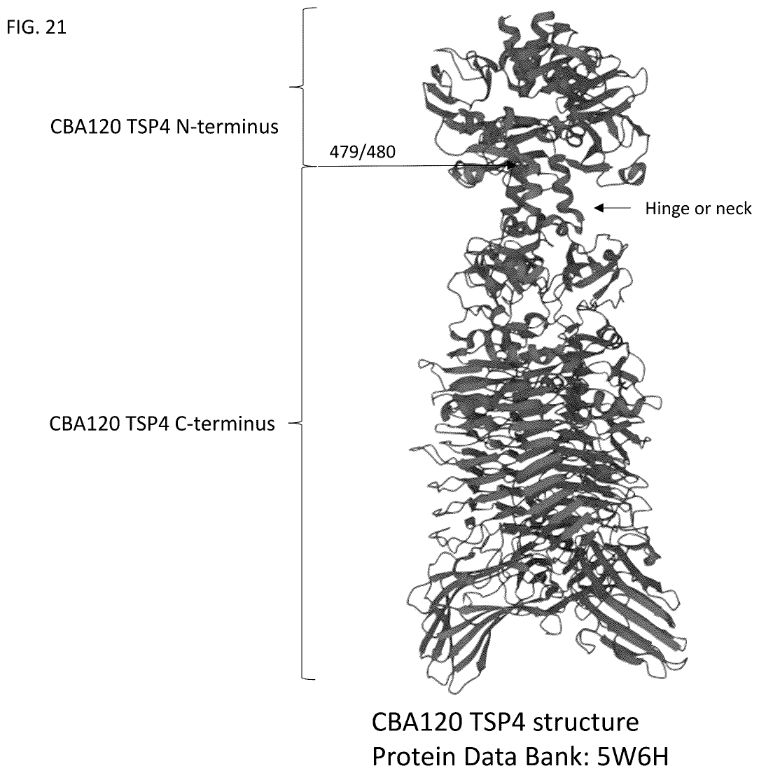

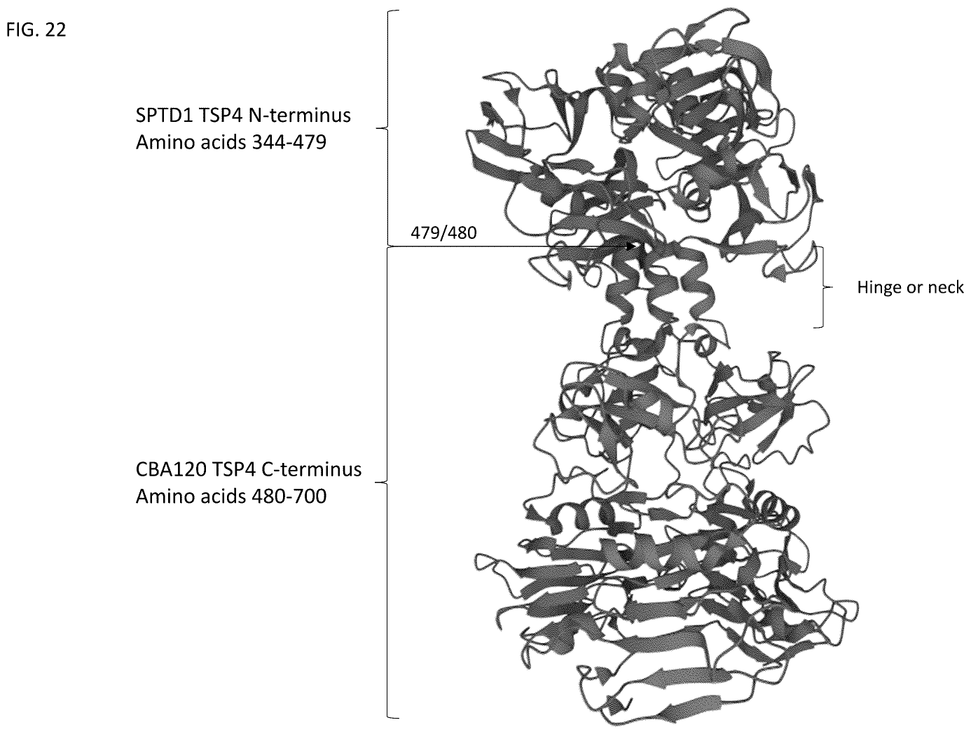

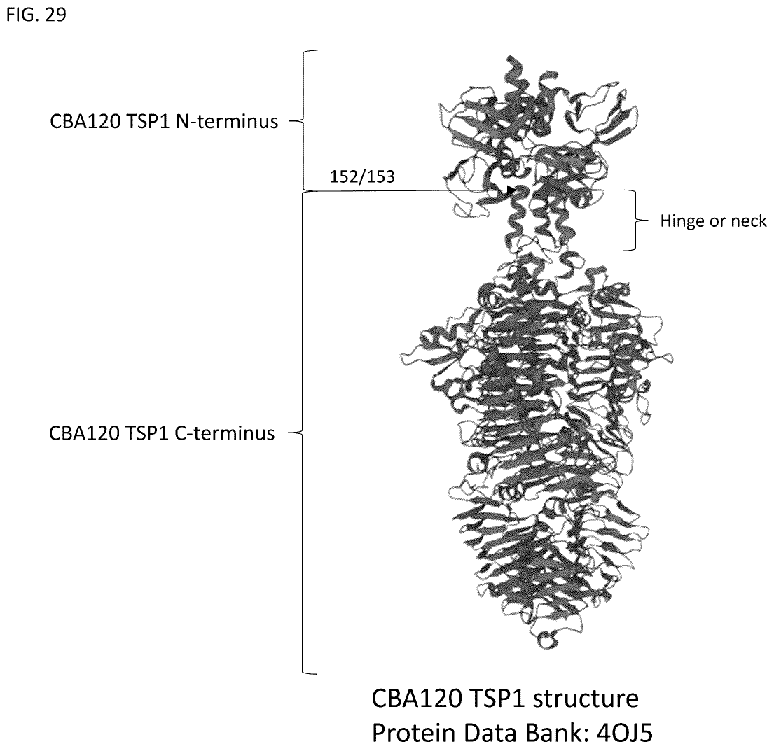

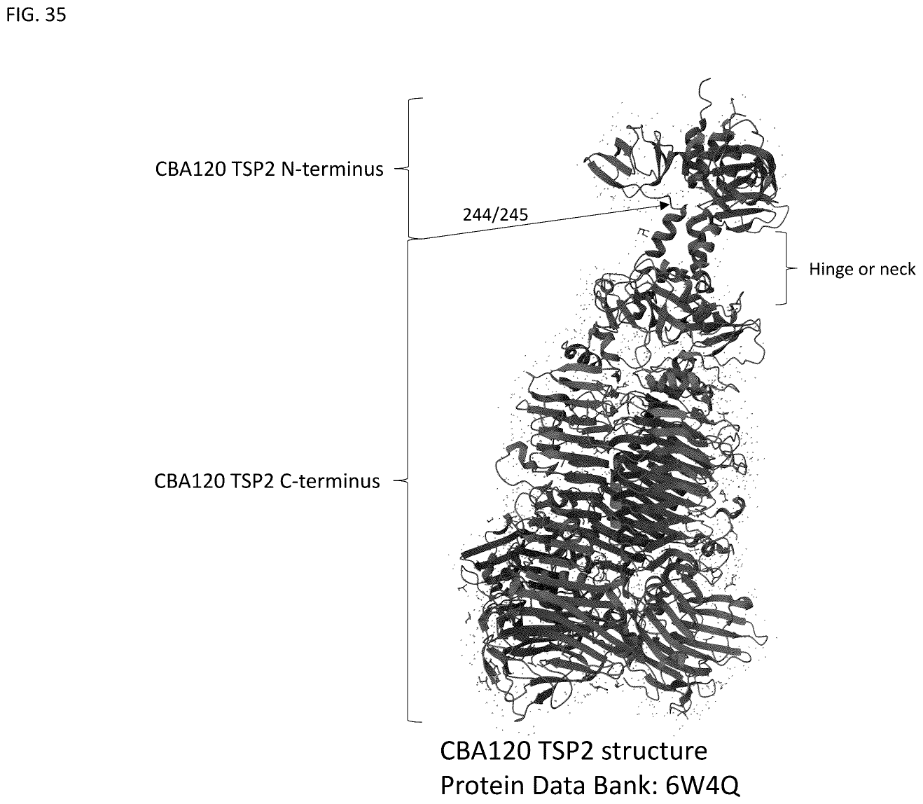

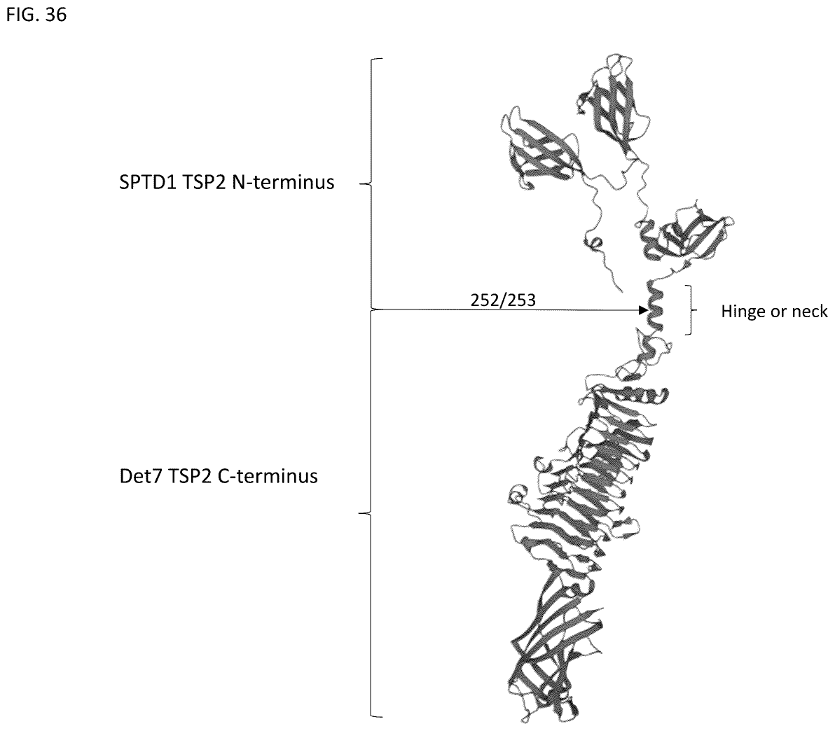

As described in more detail in the present disclosure, the compositions, methods, systems and kits according to the embodiments of the present invention may comprise one or more synthetic phages with a customized host range. In certain embodiments, a synthetic phage includes at least one recombinant or engineered tail-spike protein (TSP), which confer on the synthetic phage its host-recognition properties (which can be also described as “host range”). Engineered or recombinant TSPs according to the present disclosure are included among the embodiments of the present invention. A C-terminal region of a TSP is responsible for recognizing a phage host. For this reason, an engineered or recombinant TSP according to the present disclosure is constructed with a C-terminal region with desired recognition capacity (an ability to recognize or not recognize a particular host, or altered ability—such as lower or higher affinity—to recognize or not recognize a particular host). It is to be understood that TSP amino acid sequences or regions affecting host recognition that are described as located in the C-terminal region are not necessarily located at the C-terminal end of TSP amino acid sequence. Such may be located anywhere in C-terminal region (for example, found internally in the C-terminal region) of a TSP sequence. As currently understood, N-terminal regions or domains (which can be also referred to as “head” domains) of naturally occurring phage TSPs are involved in phage structure assembly, such as binding to phage baseplate or other TSPs, whereas the regions or domains that are C-terminal to the regions involved in phage assembly may be involved in host recognition. N-terminal region and C-terminal region of a phage TSP are connected by a so-called “neck” or “hinge” area. Exemplary structures of naturally occurring phage TSPs are illustrated and can be understood in reference to FIG. 21, FIG. 29, and FIG. 35. Exemplary structures of recombinant TSPs according to the embodiments of the present invention are illustrated and can be understood in reference to FIG. 9, FIG. 22, and FIG. 36. N-terminal region of amino acid sequence of a TSP is located N-terminally (“upstream”) of the “neck” or “hinge” region. C-terminal region of amino acid sequence of a TSP is located C-terminally (“downstream”) of the “neck” or “hinge” region.

An example of an engineered or recombinant TSP is a recombinant TSP that contains a combination of an N-terminal region and a C-terminal region that is engineered in a laboratory (that is, does not naturally occur). Both N-terminal and C-terminal regions of such a recombinant TSP may (but do not have to) contain amino acid sequences that naturally occur in N-terminal and C-terminal regions, respectively, of different phage TSPs. For instance, in recombinant TSP, the N-terminal and the C-terminal regions that occur in two different TSPs (which may be termed “parent” TSPs) are artificially combined to create a recombinant TSP with desired host-recognition properties. Even if both N-terminal and C-terminal amino acid sequences separately occur in nature, the resulting combination (which can be described “hybrid” or “chimeric” amino acid sequence) is artificial. Such a recombinant TSP may be termed “chimeric.” A chimeric TSP may include amino acid sequences from more than two (three, four, five, etc.) parent TSPs.

Another example of an engineered or recombinant TSP is a TSP that includes at least one non-naturally occurring amino acid sequence in a C-terminal region. For example, such a non-naturally occurring amino acid sequence may be artificially modified from a naturally occurring C-terminal amino acid sequence of a naturally occurring phage TSP. For example, an engineered or recombinant TSP may be constructed from a parent TSP, which may be a naturally occurring TSP or an engineered or recombinant TSP, by replacing at least a part of a C-terminal region of the parent (first) TSP with at least a part of a C-terminal region from a different (second) TSP. At least a part of the C-terminal region from the second TSP may be naturally occurring or engineered or recombinant. In this exemplary scenario, the first TSP has a different recognition capacity than the second TSP, and the resulting engineered or recombinant TSP has the recognition capacity of the second TSP. In another example, an engineered or recombinant TSP may be constructed from a parent TSP by altering one or more amino acids in a C-terminal region of the parent TSP to alter the parent TSP's recognition capacity. In one more example, an engineered or recombinant TSP includes one or more engineered sequences in its C-terminal region (in some cases, the whole amino acid sequence of the C-terminal region may be engineered de novo) to confer desired recognition capacity on the engineered TSP.

In some embodiments according to the present disclosure, the N-terminal region amino acids sequence of a recombinant or engineered TSP, such as a chimeric TSP, is derived from amino acid sequence of the N-terminal region of a TSP found in a phage used as a “parent” for a synthetic phage incorporating the chimeric TSP. Manipulating the C-terminal domain of the chimeric TSP allows to adjust (customize) its host recognition ability, while retaining the N-terminal TSP region found in the parent phage preserves the ability of the chimeric TSP to integrate into the phage structure during its assembly.

In some embodiments, including into a synthetic phage a recombinant or engineered TSP with a C-terminal region that is capable of recognizing a target host confers on the synthetic phage an ability to recognize the target host, meaning that the synthetic phage gains an ability to recognize the target host that was absent in the parent phage from which the synthetic phage was constructed. In some embodiments, including into a synthetic phage a recombinant TSP with a C-terminal region that is capable of recognizing a target host improves the ability of the synthetic phage to recognize a target host, in comparison to the parent phage from which the synthetic phage was constructed. For example, the recombinant TSP may have a higher affinity for the target host than a TSP in the parent phage. In another example, the recombinant TSP (and thus the synthetic phage) may have a higher specificity for the target host than the parent phage. In another example, a synthetic bacteriophage may be constructed to include more than one TSPs recognizing the same target host, thereby improving the ability of the synthetic bacteriophage to recognize the target host. In this scenario, more than one TSPs recognizing the same target host may be identical TSPs, non-identical TSPs recognizing the same surface receptor of the target host, or non-identical TSPs recognizing different surface receptors of the same target host. In some embodiments, including into a synthetic phage a recombinant or engineered TSP deprives the synthetic phage of an ability to recognize at least one host recognized by a parent phage from which the synthetic page was constructed, meaning that the synthetic phage loses an ability to recognize the at least one host recognized the parent phage. In some embodiments, including into a synthetic phage a recombinant TSP impairs the ability of the synthetic phage to recognize a target host, in comparison to the parent phage from which the synthetic phage was constructed. For example, the recombinant TSP (and thus the synthetic phage) may have a lower affinity for the target host than a TSP in the parent phage. In another example, the recombinant TSP (and thus the synthetic phage) may have a lower specificity for the target host than the parent phage. In the latter scenario, the synthetic phage may recognize a broader range of related hosts than the parent phage. In some embodiments, one or more native TSPs of the parental phage may be replaced with truncated versions of the TSP genes, thereby deleting the truncated TSP's bacterial specificity, while ensuring proper TSP complex formation and protein folding. For example, CBA120 TSP1 may be truncated to only the N-term region, upstream of the “neck” connecting to the C-terminal catalytic and receptor binding domain that confers specificity to Salmonella Minnesota. If the TSP complex is stabilized by the presence of the binding of TSP1's N-terminus to TSP4, including a truncated TSP1 with only the N-terminus may result in a more viable phage than simply deleting TSP1 or not supplying it in trans with a replication or TSP deficient TSP deletion construct.

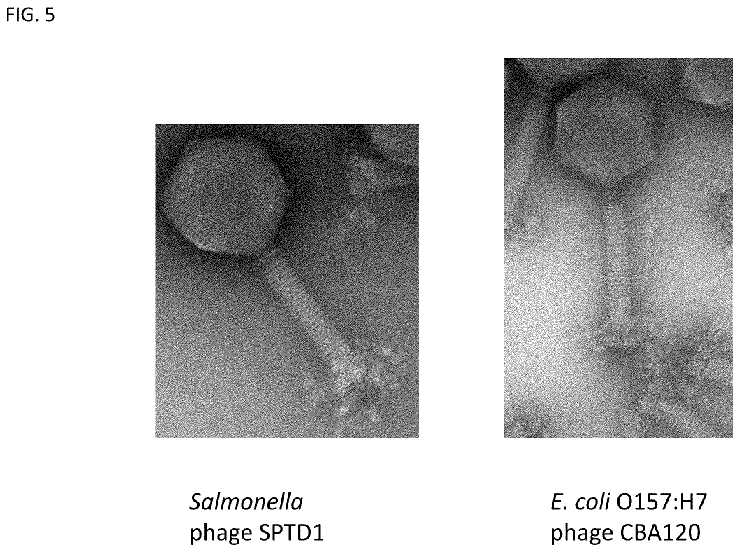

Engineered or recombinant TSPs are described in the present disclosure and are included among the embodiments of the present invention. Amino acid sequences of engineered or recombinant TSPs according to the embodiments of the present invention, as well as nucleotide sequences encoding such amino acid sequences, are included among the embodiments of the present invention. As discussed throughout the present disclosure, TSPs mediate recognition and adhesion between a phage and the surface of its host. Phage TSPs are a subtype of phage receptor binding proteins (RBPs). TSP designation is often reserved for RBPs with enzymatic activity and active penetrating “spike”-like attributes, in contrast to phage “tail fibers,” which can refer to RBPs that solely facilitate binding. In other words, a TSP can be described as a phage RBP that facilitates binding and degradation of a target host's receptor. TSPs are often characterized by distinct regions or domains, where a N-terminal (“head”) region or domain serves to bind to the phage baseplate or other TSPs and a C-terminal (“tail”) region or domain serves to bind and degrade a target host receptor. In naturally occurring phages, for example, Ackermannviridae phages, N-terminal region amino acid sequences are strongly conserved, while C-terminal region amino acid sequences are variable. C-terminal region of phage TSPs demonstrates horizontal gene transfer, with the same or similar C-terminal region amino acid sequences appearing in TSPs of multiple Ackermannviridae, and even Autographiviridae phages. For example, phages SPTD1, Det7 and P22 share the same or very similar TSP C-terminal region amino acid sequences. In another example, phage STML-13-1 shares TSP1 C-terminal amino acid sequences with phage SPTD1, TSP2 C-terminal amino acid sequences with CBA120, TSP3 C-terminal amino acid sequences with Salmonella phage Matapan, and TSP4 C-terminal amino acid sequences with Citrobacteter phage Sajours1. Although many naturally occurring TSPs are mislabeled as tail-fiber proteins (or tail fibers) in the existing literature, TSPs possess a distinct morphology that is different from the morphology of other RBPs, such as tail-fiber proteins. For example, in electron microscopy (EM) images of phages, TSPs appear as a cluster at the baseplate of a phage virion (see, for example, FIG. 5), while tail-fiber proteins appear as fibers. Each TSP is encoded by a single gene and conveys specificity to a particular host. TSPs of a phage virion form a “TSP complex” to combine the activity of multiple TSP proteins. For example, in Ackermannviridae, a complex of TSPs, confers multiple receptor-binding properties onto a phage virion (unlike typical tail-fiber proteins, which typically are identical in a phage virion).

Engineered or recombinant TSPs according to the present invention are proteins that may be incorporated into a virion of a tailed phage during virion assembly and containing a C-terminal binding domain responsible for recognition of phage host. The expression “responsible for recognition” encompasses an ability of a C-terminal region or domain of an engineered recombinant TSP to recognize a target host, a degree of an ability (which can be characterized by a binding affinity or specificity) of a C-terminal region domain of an engineered or recombinant TSP to recognize a target host, as well as a lack an ability of a C-terminal region domain of an engineered recombinant TSP to recognize a particular host. Embodiments of engineered or recombinant TSPs according to the present invention may be characterized by their three-dimensional structure. For example, in some embodiments, an engineered or recombinant TSP can be described as a homotrimeric protein that includes at least two distinctive regions or domains, an N-terminal region domain that is capable of binding to a phage tail (in some embodiments, a baseplate of a phage tail) and/or other TSPs to form a TSP complex, and a C-terminal region or domain responsible for recognition of phage host. An N-terminal region or domain and a C-terminal region domain may be connected by a “neck” or “hinge” region or domain. An N-terminal region or domain of an engineered or recombinant TSP according to the embodiments of the present invention may include or consist of between about 100 and about 600 amino acids and include or consist of between two and six sub-regions or sub-domains, which are variously involved in binding to phage baseplate or other TSPs. A C-terminal region or domain of an engineered or recombinant TSP according to the embodiments of the present invention may include or consist of between about 400 and about 1200 amino acids, possibly at least one possible catalytic sub-region or sub-domain and at least one receptor binding sub-region or sub-domain. A “neck” or “hinge” domain or regions of an engineered or recombinant TSP according to some embodiments of the present invention may include or consist of between about 5 and about 20 amino acids forming at least one alpha helix and may include the D3′ domain as described by Chao et al., 2022. An N-terminal domain, a C-terminal domain, or both, of an engineered or recombinant TSP may be derived from the same Ackermannviridae phage or different Ackermannviridae phages. For example, both an N-terminal domain and a C-terminal domain of an engineered or recombinant TSP may be derived from the same Ackermannviridae phage, and at least a C-terminal domain comprises one or more amino acid sequences that are modified from Ackermannviridae phage TSP C-terminal domain amino acid sequences that are found in nature. In another example, both an N-terminal domain and a C-terminal domain of an engineered or recombinant TSP have or are composed of amino acid sequences that are respective naturally-occurring Ackermannviridae phage amino acid sequences, but a combination of the C-terminal domain and N-terminal domain amino acid sequences does not occur in nature, because they are derived from two different Ackermannviridae phages. In another example, an N-terminal domain of an engineered or recombinant TSP includes or consists of amino acid sequences that are naturally-occurring Ackermannviridae phage amino acid sequences or are derived from (by artificial modification) from such sequences, but a C-terminal domain comprises sequences derived from a phage classified into a different family of tailed phages (that is, a non-Ackermannviridae phage), some of which are discussed elsewhere in the present disclosure. In yet another example, an C-terminal domain of an engineered or recombinant TSP includes of amino acid sequences that are naturally-occurring Ackermannviridae phage amino acid sequences or are derived from (by artificial modification) from such sequences, but a N-terminal domain comprises sequences derived from a phage classified into a different family of tailed phages (that is, a non-Ackermannviridae phage), some of which are discussed elsewhere in the present disclosure. For example, the TSP from podovirus phiAB6 in the family Autographiviridae is homologous to the TSP from myovirus Acinetobacter SH-Ab 15599 in the family Ackermannviridae, demonstrating horizontal gene transfer. Thus, amino acid sequences of TSPs from phages related to phiAB6 can be engineered into Ackermannviridae. In another example, amino acid sequences of a TSP from podovirus TR2 can be engineered into Ackermannviridae. The above examples are not intended to be limiting.

In reference to exemplary sequences of some naturally occurring TSPs of Ackermannviridae phages, an N-terminal domain is located at or comprises approximately amino acids 1-165 of CBA120's TSP1 amino acid sequence (SEQ ID:15), amino acids 1-257 of CBA120's TSP2 amino acid sequence (SEQ ID:9), amino acids 1-167 of CBA120's TSP3 amino acid sequence (SEQ ID:11), amino acids 1-479 of CBA120's TSP4 amino acid sequence (SEQ ID:13), amino acids 1-162 of SPTD1's TSP1 amino acid sequence (SEQ ID:2), amino acids 1-257 of SPTD1's TSP2 amino acid sequence (SEQ ID:3), amino acids 1-167 of SPTD1's TSP3 amino acid sequence (SEQ ID:4), amino acids 1-479 of SPTD1's TSP4 amino acid sequence (SEQ ID:5), or amino acids 1-251 of Det7 TSP2 sequence (SEQ ID:16). In reference to exemplary sequences of naturally occurring TSPs of Ackermannviridae phages, a C-terminal domain is located at or comprises approximately amino acids 166-770 of CBA120's TSP1 amino acid sequence (SEQ ID:15), amino acids 258-921 of CBA120's TSP2 amino acid sequence (SEQ ID:9), amino acids 168-627 of CBA120's TSP3 amino acid sequence (SEQ ID:11), amino acids 490-1036 of CBA120's TSP4 amino acid sequence (SEQ ID:13), amino acids 163-615 of SPTD1's TSP1 amino acid sequence (SEQ ID:2), amino acids 257-724 of SPTD1's TSP2 amino acid sequence (SEQ ID:3), amino acids 168-708 of SPTD1's TSP3 amino acid sequence (SEQ ID:4), amino acids 490-1013 of SPTD1's TSP4 amino acid sequence (SEQ ID:5), or amino acids 261-791 of Det7 TSP2 sequence (SEQ ID:16). In reference to exemplary sequences of naturally occurring TSPs of Ackermannviridae phages, a “neck” or “hinge” domain is located at or comprises approximately amino acids 155-165 of CBA120's TSP1 amino acid sequence (SEQ ID:15), amino acids 245-257 of CBA120's TSP2 amino acid sequence (SEQ ID:9), amino acids 155-168 of CBA120's TSP3 amino acid sequence (SEQ ID:11), amino acids 480-488 or 480-490 of CBA120's TSP4 amino acid sequence (SEQ ID:13), amino acids 152-162 of SPTD1's TSP1 amino acid sequence (SEQ ID:2) based on structural predictions, amino acids 247-257 of SPTD1's TSP2 amino acid sequence (SEQ ID:3), amino acids 155-168 of SPTD1's TSP3 amino acid sequence (SEQ ID:4), amino acids 480-490 of SPTD1's TSP4 amino acid sequence (SEQ ID:5). or amino acids 252-260 of Det7 TSP2 sequence (SEQ ID:16). In reference to exemplary sequences of some naturally occurring TSPs of non-Ackermannviridae phages, an N-terminal domain is located at or comprises approximately amino acids 1-362 of TR2's TSP amino acid sequence (SEQ ID:7), a C-terminal domain is located at or comprises approximately amino acids 376-875 of TR2's TSP amino acid sequence (SEQ ID:7), and “neck” or “hinge” domain is located at or comprises approximately amino acids 363-375 of TR2's TSP amino acid sequence (SEQ ID:7).

Some examples of amino acid sequences of recombinant or engineered TSPs according to the embodiments of the present invention include amino acid sequences of an N-terminal domain of SPTD1's TSP1 (SEQ ID:1), amino acid sequence or variants of the above amino acid sequence having at least 70%, at least 75%, at least 80%, at least 85%, at least 90%, at least 95%, or at least 99% sequence similarity to the above amino acid sequence, an N-terminal domain of SPTD1's TSP2 amino acid sequence (SEQ ID:3) or variants of the above amino acid sequence having at least 70%, at least 75%, at least 80%, at least 85%, at least 90%, at least 95%, or at least 99% sequence similarity to the above amino acid sequence, an N-terminal domain of SPTD1's TSP3 amino acid sequence (SEQ ID:4) or variants of the above amino acid sequence having at least 70%, at least 75%, at least 80%, at least 85%, at least 90%, at least 95%, or at least 99% sequence similarity to the above amino acid sequence, an N-terminal domain of SPTD1's TSP4 amino acid sequence (SEQ ID:5) or variants of the above amino acid sequence having at least 70%, at least 75%, at least 80%, at least 85%, at least 90%, at least 95%, or at least 99% sequence similarity to the above amino acid sequence, an N-terminal domain of CBA120's TSP1 amino acid sequence (SEQ ID:15) or variants of the above amino acid sequence having at least 70%, at least 75%, at least 80%, at least 85%, at least 90%, at least 95%, or at least 99% sequence similarity to the above amino acid sequence, an N-terminal domain of CBA120's TSP2 amino acid sequence (SEQ ID:9) or variants of the above amino acid sequence having at least 70%, at least 75%, at least 80%, at least 85%, at least 90%, at least 95%, or at least 99% sequence similarity to the above amino acid sequence, an N-terminal domain of CBA120's TSP3 amino acid sequence (SEQ ID:11) or variants of the above amino acid sequence having at least 70%, at least 75%, at least 80%, at least 85%, at least 90%, at least 95%, or at least 99% sequence similarity to the above amino acid sequence, an N-terminal domain of CBA120's TSP4 amino acid sequence (SEQ ID:13) or variants of the above amino acid sequence having at least 70%, at least 75%, at least 80%, at least 85%, at least 90%, at least 95%, or at least 99% sequence similarity to the above amino acid sequence, a C-terminal domain of SPTD1's TSP1 (SEQ ID:1), amino acid sequence or variants of the above amino acid sequence having at least 70%, at least 75%, at least 80%, at least 85%, at least 90%, at least 95%, or at least 99% sequence similarity to the above amino acid sequence, a C-terminal domain of SPTD1's TSP2 amino acid sequence (SEQ ID:3) or variants of the above amino acid sequence having at least 70%, at least 75%, at least 80%, at least 85%, at least 90%, at least 95%, or at least 99% sequence similarity to the above amino acid sequence, a C-terminal domain of SPTD1's TSP3 amino acid sequence (SEQ ID:4) or variants of the above amino acid sequence having at least 70%, at least 75%, at least 80%, at least 85%, at least 90%, at least 95%, or at least 99% sequence similarity to the above amino acid sequence, a C-terminal domain of SPTD1's TSP4 amino acid sequence (SEQ ID:5) or variants of the above amino acid sequence having at least 70%, at least 75%, at least 80%, at least 85%, at least 90%, at least 95%, or at least 99% sequence similarity to the above amino acid sequence, a C-terminal domain of CBA120's TSP1 amino acid sequence (SEQ ID:15) or variants of the above amino acid sequence having at least 70%, at least 75%, at least 80%, at least 85%, at least 90%, at least 95%, or at least 99% sequence similarity to the above amino acid sequence, a C-terminal domain of CBA120's TSP2 amino acid sequence (SEQ ID:9) or variants of the above amino acid sequence having at least 70%, at least 75%, at least 80%, at least 85%, at least 90%, at least 95%, or at least 99% sequence similarity to the above amino acid sequence, a C-terminal domain of CBA120's TSP3 amino acid sequence (SEQ ID:11) or variants of the above amino acid sequence having at least 70%, at least 75%, at least 80%, at least 85%, at least 90%, at least 95%, or at least 99% sequence similarity to the above amino acid sequence, a C-terminal domain of CBA120's TSP4 amino acid sequence (SEQ ID:13) or variants of the above amino acid sequence having at least 70%, at least 75%, at least 80%, at least 85%, at least 90%, at least 95%, or at least 99% sequence similarity to the above amino acid sequence, a C-terminal domain of Det7 amino acid sequence (SEQ ID:16) or variants of the above amino acid sequence having at least 70%, at least 75%, at least 80%, at least 85%, at least 90%, at least 95%, or at least 99% sequence similarity to the above amino acid sequence, or a C-terminal domain of TR2's TSP amino acid sequence (SEQ ID:17) or variants of the above amino acid sequence having at least 70%, at least 75%, at least 80%, at least 85%, at least 90%, at least 95%, or at least 99% sequence similarity to the above amino acid sequence. Some examples of amino acid sequences of recombinant or engineered TSPs according to the embodiments of the present invention include amino acid sequences having at least 70%, at least 75%, at least 80%, at least 85%, at least 90%, at least 95%, or at least 99% sequence similarity to SEQ ID s 6, 7, 8, 10, 12, 14, or 18. Some examples of amino acid sequences of recombinant or engineered TSPs according to the embodiments of the present comprise or consist of SEQ ID s 6, 7, 8, 10, 12, 14, or 18. Nucleic acid molecules encoding recombinant or TSPs according to the present disclosure are included among the embodiments of the present invention. Such nucleic acid molecules may be included in vectors such as bacteriophages, plasmids, phagemids, viruses, and other suitable vectors.

In some examples, synthetic phages according to the present disclosure are lytic. Lytic phages take over the machinery of the cell to make phage components. They then lyse, the cell, releasing new phage particles. Synthetic phages according to the embodiments of the present invention may be constructed from naturally occurring or recombinant (genetically modified, synthetic or engineered) phages, which are referred to as “parent.” In some examples, such parent phage, may be a naturally occurring Ackermannviridae phage or a genetically modified Ackermannviridae phage. Some non-limiting examples of naturally Ackermannviridae phages that can serve as parent phages for synthetic phages described in the present disclosure are CBA120, SPTD1, STML-13-1, KOPDS1, Salmonella phage Det7, Salmonella phage Chennai, Acinetobacter phage SH-Ab-15599, Salmonella phage Dinky, Escherichia phage PhaxI, Citrobacter phage Sajous1, Escherichia phage ECML-4, Escherichia phage Matapan, Salmonella phage Maynard, Salmonella phage ST-W77, and Salmonella phage SKML-39. A synthetic phage may have a different number of TSPs than its parent phage. A synthetic phage may include one engineered or recombinant TSP or multiple (at least two, three, or four) engineered or recombinant TSPs according to the present disclosure. In some examples, a synthetic phage includes one engineered or recombinant TSP that recognizes a target host not recognized by other TSPs of the synthetic phage. In some examples, a synthetic phage includes two engineered or recombinant TSPs that recognize two different target hosts not recognized by other TSPs of the synthetic phage. In some examples, a synthetic phage includes three engineered or recombinant TSPs that recognize three different target hosts. In some examples, a synthetic phage includes four engineered or recombinant TSPs that recognize four different target hosts. In some examples, a synthetic phage includes two or more recombinant TSPs that recognize the same target host. Nucleic acid sequences encoding synthetic phages according to the present disclosure are included among the embodiments of the present invention.

Synthetic phages according to the present disclosure may include a reporter or indicator gene. In certain embodiments, the indicator gene does not encode a fusion protein. For example, in certain embodiments, expression of the indicator gene following infection of a host microorganism, such as bacterium, results in a soluble indicator protein product. In certain embodiments, the indicator gene may be inserted into a late gene region of a synthetic phage, meaning a region of a viral genome that is transcribed late in the viral life cycle. The late gene region typically includes the most abundantly expressed genes (for example, structural proteins assembled into the bacteriophage particle). Late genes of phages can be referred to as “class III genes” and include genes with structure and assembly functions. An indicator gene may be inserted into a phage genome such that it is under the control of a phage promoter. An indicator gene may be inserted so that it replaces at least a part of a sequence of a late phage gene. Including stop codons in all three reading frames of an indicator gene may help increase expression by reducing read-through, also known as leaky expression. This strategy may also eliminate the possibility of a fusion protein being made at low levels, which would manifest as background signal that cannot be separated from the phage. Thus, in some embodiments, an indicator gene is not part of a fusion protein. That is, in some embodiments, a genetic modification may be configured such that the indicator protein product does not comprise polypeptides of the phage. In some embodiments, the non-native indicator gene is under the control of a late promoter. Using a viral late gene promoter ensures that the reporter gene (for example, luciferase) is not only expressed at high levels, like viral capsid proteins, but also does not shut down as similar endogenous bacterial genes or early bacteriophage genes. In some embodiments, the late promoter is a T4-, T7-, or ViI-like promoter, or another phage promoter similar to that found in naturally occurring phages.

An indicator gene may encode a variety of biomolecules or, in itself, may be a detectable biomolecule. For example, an indicator gene may encode a detectable polypeptide or protein. In another example, an indicator gene may be a gene that expresses a detectable product or an enzyme that produces a detectable product. In one more example, an indicator gene may encode a detectable nucleic acid or include a detectable nucleic acid. For instance, an indicator gene may encode a detectable aptameric, such us RNA Mango, or an indicator gene may contain a nucleic acid sequence detectable with real-time polymerase chain reaction (RT-PCR). In some embodiments, a product of the indicator gene can be a detectable enzyme. The indicator gene product may generate light and/or may be detectable by a color change. Various appropriate enzymes are commercially available, such as alkaline phosphatase (AP), horseradish peroxidase (HRP), or luciferase (Luc). In some embodiments, these enzymes may serve as the indicator moiety. For example, in some embodiments the indicator gene encodes a luciferase enzyme. Various types of luciferase may be used. The luciferase can be one of Oplophorus luciferase, Firefly luciferase, Lucia luciferase, Renilla luciferase, or an engineered luciferase. In some embodiments, Firefly luciferase is the indicator moiety. In some embodiments, the luciferase gene is derived from Oplophorus. Other engineered luciferases or other enzymes that generate detectable signals may also be appropriate indicator moieties.

A selection of an indicator gene to be included in a synthetic phage may be guided by a variety of considerations. For example, most phages can package DNA that is a few percent larger than their natural genome. With this consideration, a smaller indicator gene may be a more appropriate choice for modifying a bacteriophage, especially one with a smaller genome. OpLuc and NANOLUC® (Promega Corporation, Madison, Wisconsin) proteins are only about 20 kDa (approximately 500-600 bp to encode), while FLuc is about 62 kDa (approximately 1,700 bp to encode). For comparison, the genome of T7 is around 40 kbp, while the T4 genome is about 170 kbp. Moreover, the reporter gene should not be expressed endogenously by the host or hosts of the synthetic phage, should generate a high signal to background ratio, and should be readily detectable in a timely manner. NANOLUC® is a modified Oplophorus gracilirostris (deep sea shrimp) luciferase. In some embodiments, NANOLUC® combined with NANO-GLO® (Promega Corporation, Madison, Wisconsin), an imidazopyrazinone substrate (furimazine), can provide a robust signal with low background. In some embodiments, more than one indicator gene may be inserted into a synthetic phage. For example, more than one copy (such as two copies) of the same indicator gene may be inserted, which may improve signal intensity and/or signal-to-noise ratio of an assay using a synthetic phage. In another example, different indicator genes, such as two different indicator genes, may be inserted, which may allow for bimodal signal detection. For instance, NANOLUC® gene may be inserted along with a gene encoding a green fluorescent protein (GFP), or NANOLUC® gene may be inserted along with a gene encoding a different luciferase, such as firefly luciferase.

Recombinant TSPs according to the present disclosure may include a reporter or indicator moiety, such as a detectable polypeptide or protein. A detectable moiety may be an enzyme that produces a detectable product, such as, but not limited to, alkaline phosphatase (AP), horseradish peroxidase (HRP), or luciferase (Luc). A detectable product may generate light and/or may be detectable by a color change.

Compositions that include synthetic phages or recombinant TSPs according to the present disclosure may comprise one or more types of synthetic phages and/or recombinant TSPs. A composition including more than one type of synthetic phages or recombinant TSPs (two, three, four, five, six etc. types) can be referred to as “cocktail.” In some embodiments, compositions can include cocktails of different TSPs specific for different hosts of interest. In some embodiments, compositions include cocktails of different synthetic phages with recombinant TSPs specific for different hosts of interests. Such cocktails can be used for simultaneous recognition of multiple microorganisms of interest, for example, in order to simultaneously detect the presence or absence of each of microorganisms of interest in a sample. In another example, a cocktail of synthetic phages can be administered to a subject for therapeutic purposes in order to treat potential infections by multiple microorganisms. In some instances, the nature of the subject's infection may be unknown, so it is beneficial to administer a broad-spectrum synthetic phage cocktail. In some instances, it may be beneficial to administer a broad-spectrum synthetic phage cocktail preventively.

Synthetic phages and recombinant TSPs according to the present disclosure may recognize various microorganisms, including, but not limited to bacteria. As used herein, the term “bacteria” encompasses all variants of bacteria, including nonpathogenic and pathogenic bacteria. Examples of bacteria that can be recognized by synthetic phages and/or recombinant TSPs according to the present disclosure include, but are not limited to Yersinia spp, Escherichia spp, Klebsiella spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Streptomyces spp., Bacteroides spp., Prevotella spp., Clostridium spp., Bifidobacterium spp., Lactobacillus spp., Pseudomonas spp., Cronobacter spp., Shigella spp., or Campylobacter spp. In some embodiments, the bacterial cells are Bacteroides thetaiotaomicron, Bacteroides fragilis, Bacteroides distasonis, Bacteroides vulgatus, Clostridium leptum, Clostridium coccoides, Staphylococcus aureus. Bacillus subtilis, Clostridium butyricum, Brevibacterium lactofermentum, Streptococcus agalactiae, Lactococcus lactis, Leuconostoc lactis, Actinobacillus actinomycetemcomitans, Cyanobacteria, Escherichia coli, Helicobacter pylori, Selenomonas ruminantium, Shigella sonnei, Zymomonas mobilis, Mycoplasma mycoides, Treponema denticola, Bacillus thuringiensis, Staphylococcus lugdunensis, Leuconostoc oenos, Corynebacteria xerosis, Lactobacillus plantarum, Lactobacillus rhamnosus, Lactobacillus casei, Lactobacillus acidophilus, Streptococcus Enterococcus faecalis, Bacillus coagulans, Bacillus ceretus, Bacillus popilliae, Synechocystis strain PCC6S03, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, or Streptomyces ghanaenis. In some embodiments, synthetic phages according to the present disclosure can recognize bacteria of non-limiting exemplary phyla Actinobacteria, Aquificae, Armatimonadetes, Bacteroidetes, Caldiserica, Chlamydiae, Chloroflexi, Chrysiogenetes, Cyanobacteria, Deferribacteres, Deinococcus-Thermus, Dictyoglomi, Elusimicrobia, Fibrobacteres, Firmicutes (such as Bacillus, Listeria, Staphylococcus), Fusobacteria, Gemmatimonadetes, Nitrospirae, Planctomycetes, Proteobacteria (such as Acidobacillus, Aecromonas, Burkholderia, Neisseria, Shewanella, Citrobacter, Enterobacter, Erwinia, Escherichia, Klebsiella, Kluyvera, Morganella, Salmonella, Shigella. Yersinia, Coxiella, Rickettsia, Legionella, Avibacterium, Haemophilus, Pasteurella, Acinetobacter, Moraxella, Pseudomonas, Vibrio, Xanthomonas), Spirochaetes, Synergistets, Tenericutes (such as Mycoplasma, Spiroplasma, Ureaplasma), Thermodesulfobacteria and Thermotogae. Synthetic phages and recombinant TSPs according to the present disclosure may also act as archaeophages (viruses that infect archaea such as the viruses of the family Turriviridae). Thus, in some embodiments, synthetic phages or recombinant TSPs according to the present disclosure may recognize archaea.

In some embodiments, synthetic phage or recombinant TSPs are designed to optimize desirable traits for use in various applications, such as detection methods and/or methods of controlling microorganisms, some of which are described elsewhere in the present disclosure. In some embodiments bioinformatics and previous analyses of genetic modifications are employed to optimize desirable traits. For example, in some embodiments, the genes encoding TSPs can be optimized to recognize and bind to particular taxa of microorganisms, or not to recognize and bind to particular taxa of microorganisms. In other embodiments the genes encoding TSPs can be optimized to recognize and bind to an entire category of microorganisms, or a particular group within a category. In this way, recombinant TSPs and synthetic phages including them can be optimized to detect broader or narrower groups of microorganisms.

In some exemplary embodiments, Ackermannviridae phage CBA120 is altered to generate a parent phage for production of synthetic phages by generating an engineered CBA120-based phage that is capable of infecting only Escherichia coli. Such a CBA120-based phage may be engineered by including one native TSP (TSP2) from CBA120 into the CBA120-based phage. This allows for infection and propagation of CBA120-based parent phage to be performed in Escherichia coli. Synthetic phages may be constructed from such a CBA120-based parent phage also using E. coli as phage host. One or more of the three remaining TSPs (TSP1, TSP3, and TSP4) of the parent phage can be exchanged for one or more recombinant TSPs capable of recognizing one or more highly pathogenic hosts of interest (for example, B. pseudomallei or Y. pestis) without the need to perform laboratory work with such hosts. In some exemplary embodiments synthetic phages with higher specificity for target bacterial hosts are constructed by replacing off-target TSPs with second, third or fourth copies of the on-target TSP, for example, replacing CBA120's TSP1, TSP3, and TSP4 C-terminal regions with the O157:H7 specific TSP2 C-terminal region. The presence of four customizable TSPs allows for generation of phage cocktails of reduced complexity.

### Methods of Constructing Synthetic Phages and Recombinant Tail Spike Proteins

Methods of constructing synthetic phages and recombinant TSPs according to the present disclosure are included among the embodiments of the present invention. Embodiments of methods of constructing synthetic pages may begin with selection of a parent phage for constructing a synthetic phage. Parent phage may be a wild-type phage found in any environment or an engineered phage. In some embodiments, it may be preferred to utilize parent phages that have been isolated from the environment for production of synthetic phages, so that parent phages are able to recognize naturally occurring microorganisms. Any suitable method may be used to isolate a parent phage from the environment. In some embodiments, a parent phage may be specific for at least some desired target hosts, and may be further modified according to the methods of the present disclosure to alter (customize) its host range. In some examples, a parent phage may be modified to recognize additional hosts, that is, to alter a host range of the parent phage to broaden it, thereby resulting in a synthetic phage with broader host range than the parent phage. In some examples, a parent phage may be modified to recognize fewer hosts, that is, to alter a host range of the parent phage to narrow it, thereby resulting in a synthetic phage with narrower host range than the parent phage. In some examples, a parent phage may be modified to recognize a set of target hosts that differ from some or all of the hosts of the parent phage, thereby resulting in a synthetic phage with altered or customized host range. The above examples are non-limiting and other examples may be envisioned (for example, broadening the host range of the host range for one host category, and narrowing the host range of the parent phage for another host category). Methods of altering a phage host range are included among the embodiments of the present invention. Utilization of phages in various applications, such as microorganism detection or control (some of the related methods are described elsewhere in the present disclosure) is affected by phage host range. In many applications, it is desirable to employ a phage with high specificity and minimal off-target recognition. Synthetic phages according to the present disclosure can be used to enhance specificity and/or decrease off-target recognition of a parent phage.

Some embodiments of methods of constructing synthetic phages or altering a phage host range according to the embodiments of the present invention involve incorporating into a parent phage at least one recombinant TSP (for example, one, two, three, four) according to the present disclosure. In some embodiments, the incorporation of at least one recombinant TSP is performed by altering only a C-terminal region or domain of an existing TSP of the parent phage (rather than incorporating a whole gene for the recombinant TSP into the synthetic phage genome. Altering only the C-terminal region or domain of an existing TSP of the parent phage (for example, by exchanging it for a C-terminal region or domain of a TSP from a different phage, thus creating a chimeric TSP discussed elsewhere in the present disclosure) has an advantage of higher likelihood of preserving the structure of the TSP complex (since the N-terminal TSP region, which is responsible for the TSP complex assembly, remains unaltered). Also, altering only the C-terminal region of an existing TSP of the parent phage reduces the time and the costs of the genetic engineering involved. In addition, due to the presence of multiple homologous sequences in TSP N-terminal region, HR introducing only C-terminal region into a phage TSP would be less error-prone than HR introducing a whole recombinant TSP gene.

For example, a method of altering a phage host range may involve altering at least one tail-spike protein (TSP) of a parent phage, thereby generating a synthetic phage with an altered host range. In some embodiments, a method altering a phage host range may involve incorporating into a parent phage at least one recombinant TSP described elsewhere in the present disclosure, using applicable molecular biology techniques, some of which are discussed in the present disclosure. It is to be understood that constructing synthetic phages or altering a phage host range are not limited by the techniques described in the present disclosure, and any applicable techniques from the fields of molecular biology, virology, biotechnology, and related fields can be employed. For example, a phage with an altered host range may be constructed de novo based on genomic fragments or sequences derived from one or more parent phages. An engineered or synthetic phage genome may be “rebooted” generating viable phage virions. The source could be assembled DNA fragments, a cosmid insert, or yeast artificial chromosome. A fully functional synthetic phage constructed de novo may require an ability to take control of the host in order to produce phage, an origin of replication and associated replication functions, a complete set of genes permitting capsid assembly, a complete set of genes permitting tail assembly, TSPs, and packaging functions. In some embodiments, the synthetic phage with an altered host range is capable of recognizing at least one host different from the host recognized by the parent phage. In some embodiments, the synthetic phage with an altered host range is not capable of recognizing at least one host recognized by the parent phage. In some embodiments, the synthetic phage with an altered host range is capable of recognizing at least one host different from the host recognized by the parent phage, and is not capable of recognizing at least one host recognized by the parent phage.

Some embodiments of methods of constructing a synthetic phage according to the present disclosure may involve selecting a parent phage capable of infecting a microorganism, such as a bacterium, preparing a homologous recombination plasmid/vector that includes nucleic acid sequences encoding a recombinant TSP according to the present disclosure, transforming the homologous recombination plasmid/vector into the microorganism, infecting the transformed target pathogenic bacteria with the parent phage, thereby allowing homologous recombination to occur between the plasmid/vector and the phage genome; and isolating a clone of the synthetic phage that includes the recombinant TSP. Various methods for designing and preparing a homologous recombination plasmid can be used. Various methods for transforming a microorganism with a plasmid can be used, including heat-shock, F pilus mediated bacterial conjugation, electroporation, and other methods. Various methods for isolating a particular clone following homologous recombination can be used.

Some embodiments of methods of constructing a synthetic phage may involve determining the nucleic acid sequence in the region of the genome of a parent phage encoding TSPs, annotating the genome and identifying at least one TSP gene of the parent phage, and designing a sequence for homologous recombination adjacent to the identified TSP gene. Some embodiments of methods of constructing a synthetic phage may involve incorporating the sequence designed for homologous recombination into a plasmid/vector. Some embodiments of methods of constructing a synthetic phage may involve selecting for the transformed microorganisms, such as bacteria, after transforming the plasmid/vector into the microorganism. After infecting the transformed microorganisms with the parent phage and allowing homologous recombination to occur between the plasmid and the phage genome, some embodiments of methods of constructing a synthetic phage may involve determining the titer of the synthetic bacteriophage lysate. Some embodiments of methods of constructing a synthetic phage may involve performing a limiting dilution assay to enrich and isolate the synthetic phage. Some embodiments may involve repeating the limiting dilution and titer steps, following the first limiting dilution assay, as needed until the synthetic bacteriophage represent a detectable fraction of the mixture. Large scale production may be performed to obtain high titer stocks of a synthetic phage. Various methods, including, but not limited to, cesium chloride isopycnic density gradient centrifugation may be used to separate phage particles.

Methods of constructing recombinant TSPs described in the present disclosure are including among the embodiments of the present invention. Any suitable molecular biology technique or techniques may be used to generate nucleic acid sequences of recombinant TSPs. Such nucleic acid sequences may be derived from naturally occurring TSPs or artificially constructed TSPs. Naturally occurring TSP sequences may be based on (including isolated from and synthesized based on) TSP sequences of phages naturally occurring in various environments. In some embodiments, it may be advantageous to use TSP sequences from phages naturally occurring in various environments to generate recombinant TSPs capable of specifically binding naturally occurring microorganisms. Any suitable method may be used to generate a TSP nucleic acid sequence based on a phage from the environment. For example, a TSP nucleic acid sequence may be generated from an isolated genome of a naturally occurring phage. Isolated phage nucleic acids may be used for further manipulation. If phage genome sequence is unknown, various sequencing methods may be used. For example, next generation sequencing techniques may be used to generate large amounts of data (contigs) that can be used to assemble contiguous pieces of phage sequence, while the gaps may be filled using PCR-based techniques. Primers designed to anneal to the ends of contigs can be used phage nucleic acids, and the resulting PCR products can be sequenced by traditional Sanger sequencing to close the gaps between contigs. Modified Sanger sequencing may also be used to sequence phage nucleic acids.

Specificity of each TSP may be determined experimentally by comparing infectability of a panel of bacteria between a TSP swapped recombinant with its parental phage. For example, performing plaque assays on multiple bacteria strains with SPTD1 with a chimeric TSP derived from a CBA120 TSP may yield successful infection on additional strains the parental SPTD1 phage could not previously infect. Thus, it can be determined that the original CBA120 TSP was specific for these new strains. Sequence homology to TSPs with known targets may also indicate a likely bacterial target. For example, Salmonella phage STML-13-1 TSP2 has sequence homology with CBA120 TSP2, which is known to target E. coli O157:H7. Thus, it was hypothesized and subsequently experimentally confirmed by a plaque assay that STML-13-1 was able to infect E. coli O157:H7. Sequence homology among various TSPs may be determined using alignment software, such as Clustal Omega. A combination of the above and other techniques may be used to determine specificity of a specific TSP. For example, if a phage is known to infect a specific host, and three of the four TSPs on the phage are determined either experimentally and/or through homology with known TSP targets, to not infect the above host, it can be concluded that the fourth TSP of the phage is responsible for targeting the host.

TSP nucleic acid sequences derived from naturally occurring phages may be analyzed to identify nucleic acid sequence encoding a C-terminal domain of a TSP with a desired ability to recognize a host. A nucleic acid sequence encoding C-terminal domain of a TSP (“donor phage” sequence) may then be combined (“joined”) with a nucleic acid sequence encoding N-terminal domain sequence of a TSP from a parent phage, thereby generating a nucleic acid sequence encoding a recombinant TSP. A joining (or “splice”) site between the sequences may be selected based on the analysis amino acid sequences of the two proteins—a TSP from a donor phage, and a TSP from the parent phage. For example, the two sequences may be joined in the “neck” or “hinge” region of both TSPs. The location on the amino acid sequence of the “neck” or “hinge” region may be determined by multiple methods. One such method is alignment of the amino acid sequence to well characterized TSPs using Needle Pairwise Alignment, Clustal, or BLAST. As the “neck” or “hinge” region is an alpha helix, it is also possible to run the amino acid sequence in a secondary structure prediction tool, such as PHYRE2, and compare it to known or typical N-term TSP regions, aligning domains and secondary structures such as alpha helices and beta sheets. Structure matching tools, such as Swiss-Model may also be used to find homologous proteins with known three dimensional structures, at which point, the neck or domain adjacent to the neck can often be determined visually. Artificial Intelligence based folding prediction software, such as Deep Mind's AlphaFold2, or its derivatives may also be used to determine the TSP's three-dimensional structure using either the entire amino acid sequence or subsets of the sequence to minimize computation. FIG. 22 and FIG. 36 are examples of three-dimensional protein structures generated with AlphaFold2. Three dimensional structures can also be determined experimentally, with established structural biology techniques, like X-ray crystallography, Nuclear Magnetic Resonance (NMR) structural determination, or Cryo-EM (Electron Microscopy). While the domains are usually spliced between the N-term region and the “neck” from the donor TSP, splice sites may also be made between other domains to retain binding with the other TSPs, or to retain possible interactions predicted with the donor TSP. For example, two constructs were designed to replace TSP2 in a phage of interest with TSP2 from Salmonella phage Det7, one with the splice at the “neck” and the other splicing between the XD3 and TD1 domains of the N-term region, replacing the TD1 domain along with the C-term region, which structural prediction software shown to have a conformation closer to the natural Det7 TSP2. A nucleic acid molecule with a sequence encoding a recombinant TSP may be synthetized in a laboratory.

Methods of constructing synthetic pages and recombinant TSPs according to the present disclosure may involve preparing various types of plasmids, including, but not limited to, preparing plasmids for homologous recombination. Various methods and commercial products for preparing plasmids may be used. For example, PCR, site-directed mutagenesis, restriction digestion, ligation, cloning, and other techniques may be used in combination to prepare plasmids. Synthetic plasmids can also be ordered commercially (for example, GeneWiz). Cosmids can also be employed, or the CRISPR/CAS9 system could be used to selectively edit a phage genome. Some embodiments of methods of constructing synthetic pages according to the present disclosure include designing a plasmid that can readily recombine with the parent bacteriophage genome to generate recombinant genomes. In designing a plasmid, some embodiments include addition of a codon-optimized reporter gene, such as a luciferase gene. Some embodiments of constructing synthetic pages according to the present disclosure may include addition of elements into the upstream untranslated region. The untranslated region can include a promoter, such as a T4, T4-like, T7, T7-like, Salmonella- or Staphylococcus-specific bacteriophage, ViI, or ViI-like promoter. The untranslated region can also include a Ribosomal Entry/Binding Site (RBS), also known as a “Shine-Dalgarno Sequence” with bacterial systems. Either or both of these elements, or other untranslated elements, can be embedded within a short upstream untranslated region made of random sequences comprising about the same GC content as rest of the phage genome. The random region should not include an ATG sequence, as that will act as a start codon.

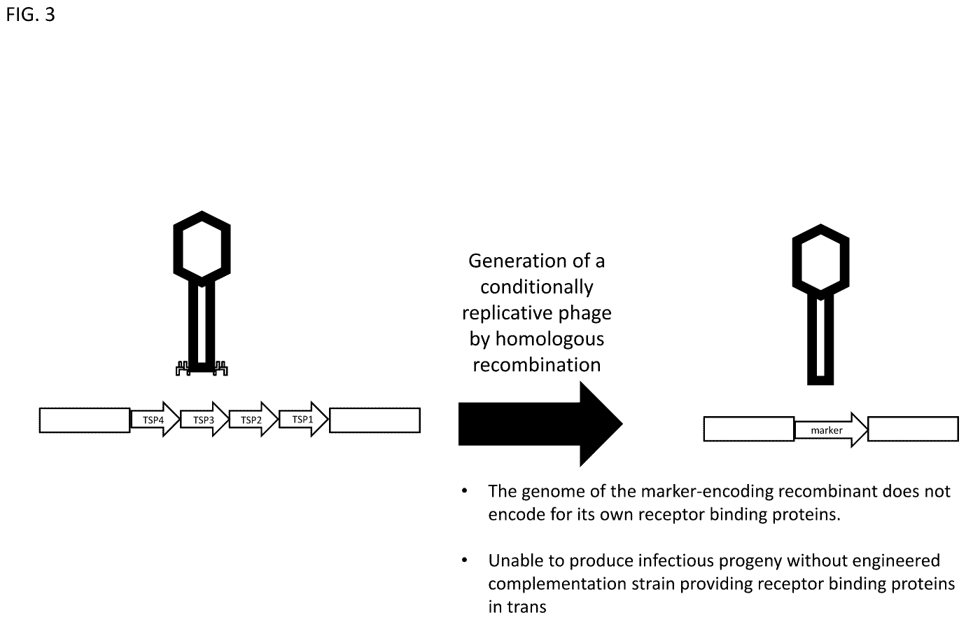

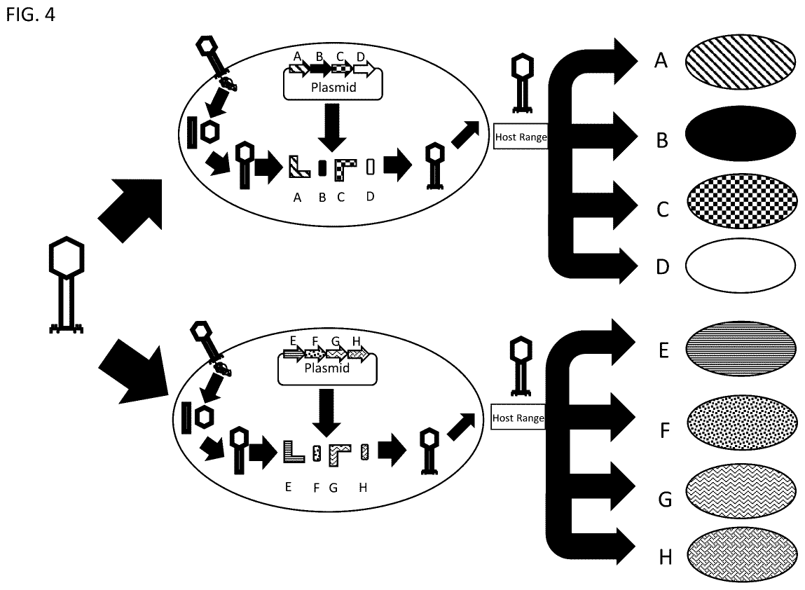

As an example of a method of constructing synthetic pages according to the present disclosure, FIG. 3 and FIG. 4 schematically illustrates the use of an Ackermannviridae phage as a customizable modular platform for constructing synthetic phages for detection of Gram-negative bacteria. As illustrated in FIG. 3, a recombinant Ackermannviridae phage deficient in TSP production is generated by deletion of the entire genomic region encoding the TSPs in an Ackermannviridae phage, such as CBA120. This is performed using homologous recombination strategy. Desired recombinants can be selected based on an expression of a marker gene, such as fluorescent marker genes, for instance, luciferase, an antibiotic resistance marker, colorimetric marker genes, and other suitable markers, but must be propagated on a complementing strain providing TSPs in trans via a transformed plasmid to produce infectious progeny. FIG. 4 is a schematic depiction of a complementing bacterial host expressing TSPs in trans to complement a recombinant phage deficient in TSP production, resulting in production of viable infectious recombinants, which are then capable of a single round of infection in wild-type bacteria. Wild-type bacteria, however, cannot support the production of infectious progeny. Progeny phage produced during infection of wild-type bacteria lack TSPs, which are essential for binding the bacterial host, as they do not contain the TSP genes in their genomes. The phage derived from bacteria with the complementing TSP-expressing plasmid has the TSP on the virions, but not the TSP-encoding genes. Thus, the original virions produced in bacteria with the complementing TSP-expressing plasmid can infect wild-type bacteria, but due to the lack of TSP genes, won't be able to generate TSP, which means that those progeny phage will not be able to infect bacteria. FIG. 4 is a schematic depiction of customizing host range of an Ackermannviridae phage using various complementing bacterial strains. Different plasmids encoding for unique TSPs allows for the assembly of recombinants with customizable host ranges.

### Method of Using Synthetic Phages and Recombinant TSPs

Methods of using synthetic phages and/or recombinant TSPs according to the present disclosure are included among the embodiments of the present invention. Embodiments of the methods according to the present disclosure can be applied to detection, including qualitative detection and quantitative detection (quantification) of a variety of microorganisms in a variety of circumstances, including but not limited to detection of microorganisms in clinical, food, water, and commercial samples. Such methods may be referred to as “detection methods.” The methods of the present invention provide high detection sensitivity and specificity and rapid detection. Embodiments of the methods according to the present disclosure can be applied to methods of controlling a microorganism or microorganisms (including, but not limited to, lowering, inhibiting or maintaining a maintaining a level, which encompasses concentration and/or quantity of a microorganism, lowering, inhibiting or maintaining proliferation and/or growth rate of a microorganism, etc.) in various contexts. For example, the methods according to the present disclosure include therapeutic methods that involve administering a synthetic phage to a subject in order to control a microorganism or microorganisms in a subject. In another example, the methods according to the present disclosure include contacting an object, a material or an apparatus with a synthetic phage in order to control a microorganisms or microorganisms on or in the object or the apparatus.

### Methods of Controlling Microorganisms

Included among the methods of controlling microorganisms by using synthetic phages are therapeutic methods. Such methods include methods for treating an infection by one or more microorganisms, including, but not limited to, bacterial infections. The methods include administering one or more synthetic phages according to the present disclosure a subject having a bacterial infection in need of treatment. It is to be understood that the methods of controlling microorganisms that involve administering one or more synthetic phages according to the present disclosure are not limited to therapeutic methods. Such methods may also be cosmetic methods, methods of improving a well-being of a subject, etc. A subject in this case may not suffer from a disease or a condition caused by a microorganism. Synthetic phages according to the embodiments of the present disclosure may be administered to a subject as a part of a pharmaceutical composition or formulations. Examples of pharmaceutical composition or formulations are described elsewhere in the present disclosure.

Diseases or conditions caused by microorganism include bacterial infections, such as, but not limited to, abdominal cramps, acne vulgaris, acute epiglottitis, arthritis, bacteremia, bloody diarrhea, botulism, Brucellosis, brain abscess, chancroid venereal disease, Chlamydia, Crohn's disease, conjunctivitis, cholecystitis, colorectal cancer, polyposis, dysbiosis, Lyme disease, diarrhea, diphtheria, duodenal ulcers, endocarditis, enteric fever, fever, glomerulonephritis, gastroenteritis, gastric ulcers, Guillain-Barre syndrome tetanus, gonorrhea, gingivitis, inflammatory bowel diseases, irritable bowel syndrome, leptospirosis, leprosy, listeriosis, tuberculosis, Lady Windermere syndrome, Legionnaire's disease, meningitis, mucopurulent conjunctivitis, multi-drug resistant bacterial infections, multi-drug resistant bacterial carriage, myonecrosis-gas gangrene, Mycobacterium avium complex, neonatal necrotizing enterocolitis, nocardiosis, nosocomial infection, otitis, periodontitis, pharyngitis, pneumonia, peritonitis, purpuric fever, Rocky Mountain spotted fever, shigellosis, syphilis, sinusitis, sigmoiditis, septicemia, subcutaneous abscesses, tularemia, tracheobronchitis, tonsillitis, typhoid fever, ulcerative colitis, urinary infection and whooping cough. A disease or a condition caused by bacteria may be a skin infections such as acne, intestinal infections such as esophagitis, gastritis, enteritis, colitis, sigmoiditis, rectitis, peritonitis, urinary tract infections, vaginal infections, female upper genital tract infections such as salpingitis, endometritis, oophoritis, myometritis, parametritis, infection in the pelvic peritoneum, respiratory tract infections such as pneumonia, intra-amniotic infections, odontogenic infections, endodontic infections, fibrosis, meningitis, bloodstream infections, nosocomial infection such as catheter-related infections, hospital acquired pneumonia, post-partum infection, hospital acquired gastroenteritis, hospital acquired urinary tract infections, and combinations thereof. In some examples, a bacterial infection is caused by a bacterium presenting an antibiotic resistance. A disease or a condition caused by bacteria may also be a metabolic disorder, for example, obesity and diabetes. A disease or a condition caused by bacteria may also be a pathology involving bacteria of an animal microbiome, including, but not limited to, inflammatory and auto-immune diseases, cancers, infections or brain disorders. For example, some bacteria of an animal microbiome, without triggering any infection, can secrete molecules that will induce and/or enhance inflammatory or auto-immune diseases or cancer development. In another example, some bacteria of the microbiome can secrete molecules that will affect the brain. Accordingly, methods of modulating an animal microbiome are included among the embodiments of the present invention. For example, synthetic phages according to the present disclosure may be administered to subjects to improve the efficacy of immunotherapies based, for example, on Chimeric Antigen Receptor T (CAR-T) cells, Tumor Infiltrating Lymphocytes (TIL), Regulatory T cells (Tregs), also known as suppressor T cells, immune checkpoint inhibitors, such as, but not limited to, programmed cell death protein 1 (PD-1) inhibitor, programmed death ligand 1 (PD-L1), and cytotoxic T lymphocyte associated protein 4 (CTLA-4).

Included among the methods of using synthetic phages according to the present disclosure are methods for personalized treatment for a subject in need of treatment for of an infection by a microorganism, including a bacterial infection. An exemplary method may involve obtaining a biological sample from the subject and determining nucleic acid (such as DNA) sequences of a group of microorganisms (such as bacteria) from the sample. Based on the determining of the sequences, one or more pathogenic microorganisms (such as bacteria) found in the sample may be identified, and a subject may be administered one or more synthetic phages according to the present disclosure, which are capable of recognizing one or more pathogenic microorganisms identified in the sample. Also included among the methods of using synthetic phages according to the present disclosure are methods that involve administering one or more synthetic phages to a subject in order to improve the effectiveness of drugs or other therapeutic agents. Some microorganisms, such as bacteria, of the microbiome, without being pathogenic by themselves, are known to be able to metabolize drugs and to modify them in ineffective or harmful molecules.

Included among the methods of controlling microorganisms by using synthetic phages according to the present disclosure are methods of controlling microorganisms in or on various objects, apparatuses, and materials. Such objects or apparatuses encompass veterinary and medical devices (including, but not limited to, medical and veterinary instruments and artificial implants, such as endoscopes) and non-medical devices and apparatuses, such as sewage pipes, devices for food preparation and storage. It is to be understood that apparatuses and devices may be found in various contexts and environments. For example, an artificial implant found in or out of a subject may be subjected to a method of controlling microorganisms according to the embodiments of the present disclosure. Also included among the methods of controlling microorganisms by using synthetic phages according to the present disclosure are methods of controlling microorganisms in or on various biological samples and materials, such as blood, plasma, cultured cells, transplants, etc. It is to be understood that biological samples and materials may have a natural origin or may be artificially produced. may be found in various contexts and environments. For example, a transplant found in or out of a subject may be subjected to a method of controlling microorganisms according to the embodiments of the present disclosure. The methods of controlling microorganisms by using synthetic phages according to the present disclosure are not limited by any particular contexts and environment. For example, such methods may include controlling microorganisms in water in various contexts, such as bioremediation and/or water purification, during food processing and food-packaging, and in agricultural industry to control bacterial contamination of crops. Methods of controlling microorganisms in or on various objects, apparatuses, materials (biological and non-biological), samples etc. include contacting one or more synthetic phages with an object, an apparatus, a material, or a sample for a period time sufficient for the one or more synthetic phages to infect and lyse one or more microorganisms found the object, the apparatus, or a sample. For example, a method may be a method of inhibiting or controlling one or more microorganisms forming a biofilm, in which an object or an apparatus is contacted with one or more synthetic phages capable of infecting one or microorganisms forming the biofilm. In another example, a method may be a method of controlling one or more organisms in an environment, in which growth of one or more microorganism is not desired or is considered to be harmful, such as medical and veterinary environments, including medical settings and operating room facilities, or food or food preparation surfaces or areas, including those where raw meat or fish are handled or discarded. The methods of controlling microorganisms may also be used to sterilize heat sensitive objects, medical devices, and tissue implants, including transplants. In some instances, only certain species or groups of microorganisms are undesired or dangerous, so synthetic phages can be designed to target these groups. For example, it may be desirable to control a particular E. coli strain, such as O157:H7, but leave other natural, non-harmful E. coli strains unaffected. Therefore, selective or whole, control of microorganisms using the methods according of the present disclosure is envisioned.

### Detection Methods

Included among the embodiments of the present invention are methods for detecting a microorganism of interest. The method may use one or more synthetic phages and/or one or more recombinant TSPs according to the present disclosure for detection of the microorganism of interest. A method may include detection of a microorganism of interest in a sample by incubating the sample with one more synthetic phages that infect one or more microorganism of interest. In some embodiments, a synthetic phage includes an indicator gene encoding an indicator gene product, as discussed elsewhere in this disclosure. The method may then include detecting the indicator gene product, with positive detection of the indicator gene product indicating that a microorganism of interest is present in the sample. In some embodiment the indicator gene product is a protein. In some embodiment the indicator gene product is a soluble protein.

An exemplary detection method according to an embodiment of the invention may be a method for detecting a microorganism, such as a bacterium, of interest in a sample, including the steps of incubating the sample with a synthetic bacteriophage that infects the microorganism of interest. The synthetic phage may include an indicator gene such that expression of the indicator gene during replication of the synthetic phage following infection of the microorganism of interest results in production of a soluble indicator protein product, and detecting the indicator protein product, wherein positive detection of the indicator protein product indicates that the microorganism of interest is present in the sample. In some embodiments, an amount of indicator moiety detected corresponds to the amount of the microorganism of interest present in the sample. In some variations of the detection method, a synthetic phage need not include an indicator gene, and may be detected by various other suitable methods, for example, by immunochemical methods and assays that utilize suitable antibodies that specifically bind to synthetic phage proteins, such as capsid proteins.

Some embodiments of the detection methods may include capturing at least one microorganism of interest prior to incubating it with a synthetic phage according to the present disclosure. For example, in some embodiments a microorganism of interest may be captured by binding a solid support, such as, but not limited to, binding to a surface of a plate, or to a filter (for example, a bacteriological filter with 0.45 m pore size spin filter or plate filter). An indicator phage is then contacted with the solid support. The solid support may be subsequently washed one or more times to remove excess unbound synthetic phage. In an embodiment, a medium (for example, Luria-Bertani Broth (LB broth), or Tryptic Soy Broth or Tryptone Soy Broth (TSB), or Buffered Peptone Water (BPTW) may be added for further incubation time, to allow replication synthetic phage and high-level expression of the gene encoding the indicator moiety. The incubation step may only need to be long enough for a single phage life cycle. A single replication cycle of a synthetic phage can be sufficient for sensitive and rapid detection of a target microorganism. Soluble indicator released into the surrounding liquid upon lysis of the bacteria may then be measured and quantified. Alternatively, the indicator signal may be measured directly on the solid support. For example, when an indicator moiety is an enzyme, such as luciferase or HRP, an indicator substrate may be incubated with the portion of the sample that remains on a solid support, such as bound to a filter or a plate surface. Accordingly, in some embodiments the enzymatic reaction may be detected by placing the plate, such as a 96-well plate, directly in a suitable detector device.

Lysis of cells of a microorganism of interest, such as a bacterium, may occur before, during, or after the detection step. Infected unlisted cells may be detectable upon addition of an indicator substrate in some embodiments, for example, when an indicator exits cells and/or a substrate enter cells without complete cell lysis. Thus, for embodiments utilizing a spin filter system, where only an indicator released into lysate is analyzed, lysis is required for detection. However, for embodiments utilizing filter plates or 96-well plates with sample in solution or suspension, where the original plate full of intact and lysed cells is directly assayed, lysis is not necessary for detection. In some embodiments, the reaction of indicator moiety with substrate may continue for 30 minutes or more, and detection at various time points may be desirable for optimizing sensitivity. For example, in embodiments using 96-well lates as the solid support, detector readings may be taken initially and at 10- or 15-minute intervals until the reaction is completed.

A detection method may be performed to utilize a general concept that can be modified to accommodate different sample types or sizes and assay formats. Embodiments of the methods may have total performance times to detection (which may be referred to as “assay times”) under 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 21.0, 22.0, 23.0, 24.0, 25.0, or 26.0 hours, depending on the sample type, sample size, cell concentration in the sample, and assay format. For example, the amount of time required may be somewhat shorter or longer depending on the synthetic phage and the microorganism, such as a bacterium, to be detected in the assay, type, and size of the sample to be tested, conditions required for viability of the target, complexity of the physical/chemical environment, and the concentration of “endogenous” non-target contaminants.

In an exemplary method, a synthetic phage encodes soluble luciferase and is engineered to express soluble luciferase during replication of the phage. Expression of luciferase is driven by a viral capsid promoter (for example, bacteriophage T5 or T4 late promoter), yielding high expression. Parental phage is substantially purified of luciferase, so the luciferase detected in the assay mostly originates from replication of progeny phage during infection of the cells of a microorganism being detected. Thus, there is no need to separate out the parental phage from the progeny phage. At least part of a sample that may contain a microorganism of interest is placed in a spin column filter and centrifuged to remove liquid, and an appropriate multiplicity of synthetic phages are added. The infected cells may be incubated for a time sufficient for replication of progeny phage and cell lysis to occur (such as 30-120 minutes at 37° C.). The parental and progeny phage plus free luciferase in the lysate may then be collected, for example, by centrifugation, and the level of luciferase in the filtrate quantified using a luminometer. Alternatively, a high throughput method may be employed, where in which samples are applied to a 96-well filter plate, and, after the manipulations listed above are performed, may be directly assayed for luciferase in the original 96-well filter plate without a final centrifugation step.

In another exemplary method, a filter plate assay for detecting a microorganism, such as a bacterium, of interest using a synthetic phage is performed. Briefly, samples that include a microorganism of interest may be added to wells of a multi-well filter plate and spun to concentrate the samples by removal of liquid from the sample. Synthetic phages are added to wells and incubated with additional media added for enough time sufficient for adsorption followed by infection of target microorganism and advancement of the phage life cycle (for example, ˜ 45 minutes). Finally, a luciferase substrate is added and reacts with any luciferase present. The resulting emission is measured in a luminometer, which detects luciferase activity.

In some examples, a detection may be performed without concentrating microorganisms on or near the capture surface. In such examples, the samples are not concentrated, but are simply incubated directly with a synthetic phage for a period of time and subsequently assayed for indicator activity. Aliquots of synthetic phage may be distributed to the individual wells of a multi-well plate, and then test sample aliquots that may contain a microorganism of interest are added and incubated for a period of time sufficient for synthetic phage to replicate and generate soluble indicator protein. The plate wells containing soluble indicator and phage may then be assayed by an appropriate method to measure the indicator activity on the plate.

In some embodiments, the sample may be enriched prior to testing by incubation in conditions that encourage growth. In such embodiments, the enrichment period can be 1, 2, 3, 4, 5, 6, 7, 8, 9, 10, 11, 12, 13, 14, 15, 16, 17, 18, 19, 20, 21, 22, 23, 24, hours or longer, depending on the sample type and size. In some embodiments, a synthetic phage includes a detectable indicator moiety, and infection of a single cell of a microorganism of interest, such as a bacterial cell, can be detected by an amplified signal generated via the indicator moiety. In some embodiments a synthetic phage is added to the sample at a concentration sufficient to rapidly find, bind, and infect a microorganism of interest present in very low numbers in the sample, such as a single cell. In some embodiments, synthetic phage concentration can be sufficient to find, bind, and infect a microorganism of interest in less than one hour. In other embodiments, these events can occur in less than two hours, or less than three hours, following addition of a synthetic phage to the sample. For example, in certain embodiments, synthetic phage concentration for the incubating step is greater than 1×10⁵ PFU/mL, greater than 1×10⁶ PFU/mL, or greater than 1×10⁷ PFU/mL.

A synthetic phage may be purified so as to be substantially free of any residual indicator protein that may be generated upon production of the infectious agent stock. Thus, in certain embodiments of the detection methods, a synthetic phage may be purified using cesium chloride isopycnic density gradient centrifugation prior to incubation with the sample, or by other suitable methods. In some embodiments of the detection methods, a microorganism may be detected without any isolation or purification of the microorganisms from a sample. For example, in certain embodiments, a sample containing one or a few microorganisms of interest may be applied directly to an assay container such as a spin column, a microtiter well, or a filter and the assay is conducted in that assay container. Aliquots of a sample may be distributed directly into wells of a multi-well plate, a synthetic phage may be added, and after a period of time sufficient for infection, a lysis buffer may be added as well as a substrate for the indicator protein and assayed for detection of the indicator signal. Some embodiments of the method can be performed on filter plates. Some embodiments of the detection method can be performed with or without concentration of the sample before infection with a synthetic phage. Methods of the invention may comprise various other steps to increase sensitivity. For example, as discussed in more detail herein, the method may comprise a step for washing the captured and infected bacterium, after adding the bacteriophage but before incubating, to remove excess parental bacteriophage and/or luciferase or other reporter protein contaminating the bacteriophage preparation.

In some embodiments of detection methods, detection of the microorganism of interest may be completed without the need for culturing the sample as a way to increase the population of the microorganisms. For example, in certain embodiments the total time required for detection is less than 26.0 hours, 25.0 hours, 24.0 hours, 23.0 hours, 22.0 hours, 21.0 hours, 20.0 hours, 19.0 hours, 18.0 hours, 17.0 hours, 16.0 hours, 15.0 hours, 14.0 hours, 13.0 hours, 12.0 hours, 11.0 hours, 10.0 hours, 9.0 hours, 8.0 hours, 7.0 hours, 6.0 hours, 5.0 hours, 4.0 hours, 3.0 hours, 2.5 hours, 2.0 hours, 1.5 hours, 1.0 hour, 45 minutes, or less than 30 minutes. Some embodiments of the detection methods may detect <10 cells of the microorganism (such as, 1, 2, 3, 4, 5, 6, 7, 8, 9 microorganisms) present in a sample. In certain embodiments, the detection methods as few as 2, 3, 4, 5, 6, 7, 8, 9, 10, 15, 20, 30, 40, 50, 60, 70, 80, 90, or 100 of cells of microorganisms in the sample.

Some detection methods according to the embodiments of the present invention use of a binding agent (for example, an antibody) to purify and/or concentrate a microorganism of interest from the sample in addition to detection with a synthetic phage or a recombinant TSP according to the present disclosure. For example, in certain embodiments, the present invention comprises a method for detecting a microorganism of interest in a sample comprising the steps of: capturing the microorganism from the sample on a solid support using a capture antibody specific to the microorganism of interest incubating the sample with a synthetic phage that infects the microorganism of interest, wherein the synthetic phage comprises an indicator gene inserted into a late gene region of the synthetic phage, such that expression of the indicator gene during synthetic phage replication following infection of the microorganism of interest results in a soluble indicator protein product, and detecting the indicator protein product, wherein positive detection of the indicator protein product indicates that the microorganism of interest is present in the sample.

Various applications of the detection methods according to the present disclosure are envisioned. For example, detection methods can be employed to test initial patient samples for the presence of particular pathogens, such as a particular genus or species of bacterium. In some embodiments, a method be used to detect a particular pathogen in a clinical sample. A method may be used to evaluate potential efficacy of treatment in the context of a given patient's infection or other pathogenic medical condition. In another example, detection methods may be applied to non-clinical uses. For example, detection methods may be used as food safety detection methods to identify the presence of a particular bacteria in food. In another examples, detection methods can be used to detect pathogens in patient samples subsequent to the initiation of some type of treatment, such as antibiotic treatment, or some other type of drug treatment or therapy. A detection can be used to monitor the progress or efficacy of any type of treatment or therapy.

### Systems and Kits

Systems and kits are included among embodiments of the present invention. In some embodiments, systems (for example, automated systems) or kits according to the present disclosure include components for performing the methods according to the present disclosure, such are reagents, materials, or apparatuses. The term “component” is broadly defined and includes any suitable apparatus or collections of apparatuses, reagent or a plurality of reagents, that suitable for carrying out a method according to the present disclosure. The components need not be integrally connected or situated with respect to each other in any particular way. The invention includes any suitable arrangements of the components with respect to each other. For example, the components need not be in the same room. But in some embodiments, the components are connected to each other in an integral unit. In some embodiments, the same components may perform multiple functions. Some embodiments of the systems or kits include at least one synthetic phage according to the present disclosure, and/or at least one recombinant TSP according to the present disclosure. Some embodiments of the systems and systems are particularly suitable for automation and/or performing the methods according to the present disclosure in high-throughput manner. In certain embodiments, a system or a kit may comprise a self-contained unit that is deliverable from a first site to a second site.

In some embodiments, the invention includes systems or kits for detection of a microorganism of interest in a sample. Such system and/or kits may be referred to as diagnostic systems and/or kits, or detection systems and/or kits. The systems or kits may in certain embodiments comprise a component for incubating the sample with a synthetic phage or a plurality of synthetic phages specific for the microorganism or microorganisms of interest. The synthetic phage may include an indicator gene, and a system or a kit may include one or more components for detecting a product of the indicator gene (for example, an enzyme substrate, when a product of the indicator gene is an enzyme). In some embodiments of both the systems and the kits, a synthetic phage is capable of specifically infecting a microorganism of interest and comprises an indicator gene inserted into a late gene region, such that expression of the indicator gene during the infection of the microorganism results in a soluble indicator protein product. Some systems and kits may include a component for capturing the microorganism of interest on a solid support. The systems or kits may in certain embodiments include an apparatus including a solid support, which in turn includes a cell-binding component. The systems or kits may in certain embodiments include a signal detecting component capable of detecting the indicator gene product produced from infecting a microorganism in a sample with the synthetic phage. In some examples, the signal detecting component is a luminometer, which can be a handheld device. A system or a kit may further include vessels or containers that contain substrate and/or media. In certain embodiments, a system and/or a kit may include a component for washing the captured microorganism sample. In some embodiments, a system or a kit may include a component for isolating the microorganism of interest from the other components in the sample. In some detections systems and/or kits according to the embodiments of the present invention, the steps performed by a system or a kit are automated or controlled by the user via computer input. A liquid-handling robot performs at least one step. In a computerized system, the system may be fully automated, semi-automated, or directed by the user through a computer (or some combination thereof).

In an exemplary embodiment, a system or a kit includes components for detecting a microorganism of interest a sample. Such a system or a kit may include a component for isolating at least one microorganism from other components in the sample, a component for infecting at least one microorganism with one or more synthetic phages, a component for lysing the at least one infected microorganism to release a synthetic phage present in the microorganism, and a component for detecting the synthetic phage, for example, a soluble protein encoded and expressed by an indicator gene included in the synthetic phage. When such an exemplary system or a kit is used to perform a detection method according to the present disclosure, detection a soluble protein product of the synthetic phage indicates that the microorganism is present in the sample.

In some embodiments, the invention includes systems or kits involved in performing methods of controlling microorganisms or a microorganism of interest a microorganism in various contexts, some of which are described in the present disclosure. For example, a kit may include one or more synthetic phages or a composition including one or more synthetic phages according to the present disclosure, and a container for its storage, such as a bag or a vial. Such a container may have a sterile access port, for example, a bag or vial having a stopper pierceable by a hypodermic injection needle. In another example, a kit may include one or more synthetic phages or a composition including one or more synthetic phages according to the present disclosure in lyophilized or concentrated form and diluent. In such a kit, a diluent may also be a pharmaceutically acceptable carrier or excipient, as described elsewhere in the present disclosure. Non-limiting examples of diluents that may be included in such a kit are saline, buffered saline, water, or sucrose. In another example, a kit may include one or more synthetic phages or a composition including one or more synthetic phages according to the present disclosure and a device for administering one or more synthetic phages or a composition including one or more synthetic phages according to the present disclosure, or a device or bringing one or more synthetic phages or a composition including one or more synthetic phages according to the present disclosure in contact with an object or an apparatus. A device may be a syringe for injection or oral administration (for example, the kit may be a syringe pre-filled with a liquid composition), a microneedle device, such as a microneedle patch, an inhaler, a nebulizer, a sprayer, or a pump. A kit may contain multiple devices.

### Compositions

Compositions including at least one synthetic phage according to the present disclosure, according to the present disclosure are included among the embodiments of the present invention. In some examples, the compositions are pharmaceutical compositions, which include one or more synthetic phages according to the present disclosure. For pharmaceutical use, one or more synthetic phages according to the present disclosure may be formulated as a pharmaceutical preparation or compositions including at least one pharmaceutically acceptable carrier, diluent or excipient (the foregoing terms may be used interchangeably. Pharmaceutical compositions include compositions suitable for therapeutic use, as well for non-therapeutic uses, such as, but not limited to, a cosmetic use or a use for improving the well-being of a subject. Pharmaceutical compositions may further include one or more pharmaceutically active compounds. A pharmaceutical composition may be in a form suitable for oral administration, for parenteral administration (such as by intravenous, intramuscular or subcutaneous injection or intravenous infusion), for topical administration, for administration by inhalation, by a skin patch, by an implant, by a suppository, etc. An administration form (which may also be referred to as a “dosage form”) may be solid, semi-solid or liquid, depending on the manner and route of administration. For example, a formulation for oral administration may be provided with an enteric coating that will allow one or more synthetic phages in the formulation to resist the gastric environment and pass into the intestines. More generally, synthetic formulations for oral administration may be suitably formulated for delivery into any desired part of the gastrointestinal tract. Suppositories may be used for delivery into the gastrointestinal tract.

A composition according to the present disclosure may include a carrier or a vehicle, such as a pharmaceutically acceptable carrier vehicle. A solid pharmaceutically acceptable vehicle may include one or more substances which may also act as flavoring agents, lubricants, solubilizers, suspending agents, dyes, fillers, glidants, compression aids, inert binders, sweeteners, preservatives, dyes, coatings, or tablet-disintegrating agents. Suitable solid vehicles include, for example calcium phosphate, magnesium stearate, talc, sugars, lactose, dextrin, starch, gelatin, cellulose, polyvinylpyrrolidone, low melting waxes and ion exchange resins. A pharmaceutical composition may be prepared as a sterile solid composition that may be suspended at the time of administration using sterile water, saline, or other appropriate sterile injectable medium. A pharmaceutical compositions may be administered in the form of a sterile solution or suspension containing other solutes or suspending agents (for example, enough saline or glucose to make the solution isotonic), bile salts, acacia, gelatin, sorbitan monoleate, a polysorbate, oleate esters of sorbitol and its anhydrides copolymerized with ethylene oxide etc. Pharmaceutical compositions may be administered orally either in liquid or solid composition form. Compositions suitable for oral administration include solid forms, such as pills, capsules, granules, tablets, and powders, and liquid forms, such as solutions, syrups, elixirs, and suspensions. Forms useful for enteral administration include sterile solutions, emulsions, and suspensions.

One or more synthetic phages according to the present disclosure may be dissolved or suspended in a liquid vehicle such as water, an organic solvent, a mixture of both or pharmaceutically acceptable oils or fats. A liquid vehicle can contain other suitable additives such as solubilizers, emulsifiers, buffers, preservatives, sweeteners, flavoring agents, suspending agents, thickening agents, colors, viscosity regulators, stabilizers or osmo-regulators. Suitable examples of liquid vehicles include water (which may include various additives for example, cellulose derivatives, such as sodium carboxymethyl cellulose solution), alcohols (including monohydric alcohols and polyhydric alcohols) and their derivatives, and oils. A liquid carrier or vehicle can also be an oily ester such as ethyl oleate and isopropyl myristate. A liquid vehicle for pressurized compositions can be a halogenated hydrocarbon or other pharmaceutically acceptable propellant. For external (such as wound, dermal, or transdermal) administration to a subject, a composition can be formulated into ointment, cream or gel form and appropriate penetrants or detergents may be used to facilitate permeation, such as dimethyl sulfoxide, dimethyl acetamide and dimethylformamide. For transmucosal administration, nasal sprays, rectal or vaginal suppositories can be used. A composition can include various suppository bases, such as, but not limited to, cocoa butter, polyethylene glycol carbowaxes), polyethylene sorbitan monostearate, and mixtures of these with other compatible materials to modify the melting point or dissolution rate.

In some embodiments, a composition can be prepared in a dry form (for example, dehydrated form), such as a lyophilized form. Such a formulation can be referred to as “lyophilized” or a “lyophilizate.” Lyophilization is a process of or freeze-drying, during which a solvent is removed from a liquid formulation. Lyophilization process may include one or more of simultaneous or sequential steps of freezing and drying. A composition be lyophilized in an aqueous solution comprising a nonvolatile or volatile buffer. Non-limiting examples of suitable nonvolatile buffers are PBS, Tris-HCl, HEPES, or L-Histidine buffer. Non-limiting examples of suitable volatile buffers are ammonium bicarbonate, Ammonia/acetic acid, or N-ethylmorpholine/acetate buffer. A lyophilized composition can include appropriate carriers or excipients. Such appropriate excipients may include, but are not limited to, a cryo-preservative, a bulking agent, a surfactant, or their combinations. Exemplary excipients include one or more of a polyol, a disaccharide, or a polysaccharide, such as, for example, mannitol, sorbitol, sucrose, trehalose, and/or dextran 40. In some instances, the cryo-preservative may be sucrose and/or trehalose. In some instances, the bulking agent may be glycine or mannitol. In one example, the surfactant may be a polysorbate such as, for example, polysorbate-20 and/or polysorbate-80. A lyophilized composition can be, for example, in a cake or powder form. A lyophilized compositions may be rehydrated/solubilized/reconstituted in a carrier or excipient (such as water or buffer solution) prior to use.

### Computer Systems and Computer Readable Media

A system or a kit a described in the present disclosure, or any of its components, may be embodied in the form of a computer system. Typical examples of a computer system include a general-purpose computer, a programmed microprocessor, a microcontroller, a peripheral integrated circuit element, and other devices or arrangements of devices that are capable of implementing the steps that constitute the method of the present technique.

A computer system may comprise a computer, an input device, a display unit, and/or the Internet. The computer may further comprise a microprocessor. The microprocessor may be connected to a communication bus. The computer may also include a memory. The memory may include random access memory (RAM) and read only memory (ROM). The computer system may further comprise a storage device. The storage device can be a hard disk drive or a removable storage drive such as a floppy disk drive, optical disk drive, etc. The storage device can also be other similar means for loading computer programs or other instructions into the computer system. The computer system may also include a communication unit. The communication unit allows the computer to connect to other databases and the Internet through an I/O interface. The communication unit allows the transfer to, as well as reception of data from, other databases. The communication unit may include a modem, an Ethernet card, or any similar device which enables the computer system to connect to databases and networks such as LAN, MAN, WAN and the Internet. The computer system thus may facilitate inputs from a user through input device, accessible to the system through I/O interface.

A computing device typically will include an operating system that provides executable program instructions for the general administration and operation of that computing device, and typically will include a computer-readable storage medium (for example, a hard disk, random access memory, read only memory, etc.) storing instructions that, when executed by a processor of the server, allow the computing device to perform its intended functions. Suitable implementations for the operating system and general functionality of the computing device are known or commercially available, and are readily implemented by persons having ordinary skill in the art, particularly in light of the disclosure herein.

The computer system executes a set of instructions that are stored in one or more storage elements, in order to process input data. The storage elements may also hold data or other information as desired. The storage element may be in the form of an information source or a physical memory element present in the processing machine.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art. Similarly, any necessary files for performing the functions attributed to the computers, servers, or other network devices may be stored locally and/or remotely, as appropriate. Where a system includes computing devices, each such device can include hardware elements that may be electrically coupled via a bus, the elements including, for example, at least one central processing unit (CPU), at least one input device (for example, a mouse, keyboard, controller, touch screen, or keypad), and at least one output device (for example, a display device, printer, or speaker). Such a system may also include one or more storage devices, such as disk drives, optical storage devices, and solid-state storage devices such as random access memory (“RAM”) or read-only memory (“ROM”), as well as removable media devices, memory cards, flash cards, etc.

Such devices also can include a computer-readable storage media reader, a communications device (for example, a modem, a network card (wireless or wired), an infrared communication device, etc.), and working memory as described above. The computer-readable storage media reader can be connected with, or configured to receive, a computer-readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or Web browser. It should be appreciated that alternate embodiments may have numerous variations from that described above. For example, customized hardware might also be used and/or particular elements might be implemented in hardware, software (including portable software, such as applets), or both. Further, connection to other computing devices such as network input/output devices may be employed.

Non-transient storage media and computer readable media for containing code, or portions of code, can include any appropriate media known or used in the art, including storage media and communication media, such as but not limited to volatile and non-volatile, removable and non-removable media implemented in any method or technology for storage and/or transmission of information such as computer readable instructions, data structures, program modules, or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices, or any other medium which can be used to store the desired information and which can be accessed by the a system device. Based on the disclosure and teachings provided herein, a person of ordinary skill in the art will appreciate other ways and/or methods to implement the various embodiments.

A computer-readable medium may comprise, but is not limited to, an electronic, optical, magnetic, or other storage device capable of providing a processor with computer-readable instructions. Other examples include, but are not limited to, a floppy disk, CD-ROM, DVD, magnetic disk, memory chip, ROM, RAM, SRAM, DRAM, content-addressable memory (“CAM”), DDR, flash memory such as NAND flash or NOR flash, an ASIC, a configured processor, optical storage, magnetic tape or other magnetic storage, or any other medium from which a computer processor can read instructions. In one embodiment, the computing device may comprise a single type of computer-readable medium such as random access memory (RAM). In other embodiments, the computing device may comprise two or more types of computer-readable medium such as random access memory (RAM), a disk drive, and cache. The computing device may be in communication with one or more external computer-readable mediums such as an external hard disk drive or an external DVD or Blu-Ray drive.

As discussed above, the embodiment comprises a processor which is configured to execute computer-executable program instructions and/or to access information stored in memory. The instructions may comprise processor-specific instructions generated by a compiler and/or an interpreter from code written in any suitable computer-programming language including, for example, C, C++, C #, Visual Basic, Java, Python, Perl, JavaScript, and ActionScript (Adobe Systems, Mountain View, Calif). In an embodiment, the computing device comprises a single processor. In other embodiments, the device comprises two or more processors. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

The computing device comprises a network interface. In some embodiments, the network interface is configured for communicating via wired or wireless communication links. For example, the network interface may allow for communication over networks via Ethernet, IEEE 802.11 (Wi-Fi), 802.16 (Wi-Max), Bluetooth, infrared, etc. As another example, network interface may allow for communication over networks such as CDMA, GSM, UMTS, or other cellular communication networks. In some embodiments, the network interface may allow for point-to-point connections with another device, such as via the Universal Serial Bus (USB), 1394 FireWire, serial or parallel connections, or similar interfaces. Some embodiments of suitable computing devices may comprise two or more network interfaces for communication over one or more networks. In some embodiments, the computing device may include a data store in addition to or in place of a network interface.

Embodiments of suitable computing devices may comprise or be in communication with a number of external or internal devices such as a mouse, external memory, a CD-ROM, DVD, a keyboard, a display, audio speakers, one or more microphones, or any other input or output devices. For example, the computing device may be in communication with various user interface devices and a display. The display may use any suitable technology including, but not limited to, LCD, LED, CRT, and the like.

The set of instructions for execution by the computer system may include various commands that instruct the processing machine to perform specific tasks such as the steps that constitute the method of the present technique. The set of instructions may be in the form of a software program. Further, the software may be in the form of a collection of separate programs, a program module with a larger program or a portion of a program module, as in the present technique. The software may also include modular programming in the form of object-oriented programming. The processing of input data by the processing machine may be in response to user commands, results of previous processing, or a request made by another processing machine.

While the present invention has been disclosed with references to certain embodiments, numerous modifications, alterations and changes to the described embodiments are possible without departing from the scope and spirit of the present invention, as defined in the appended claims. Accordingly, it is intended that the present invention not be limited to the described embodiments, but that it have the full scope defined by the language of the following claims, and equivalents thereof.

### Examples

The following examples are offered to illustrate, but not to limit the claimed invention.

### Example 1: Construction and Testing of Synthetic Phage with Recombinant TSP3

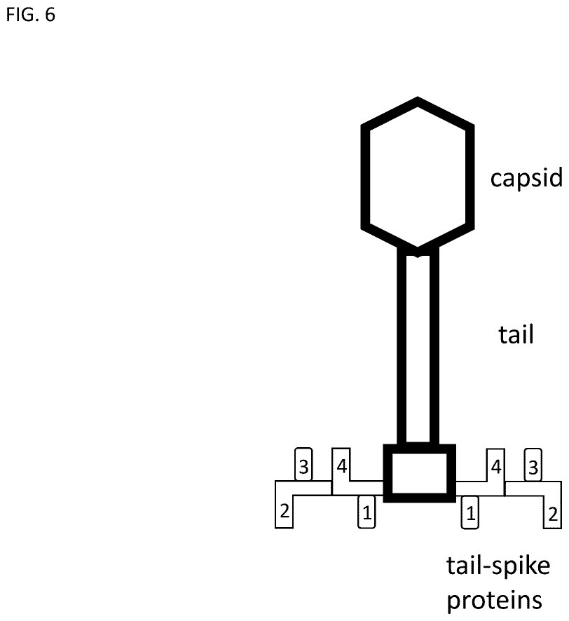

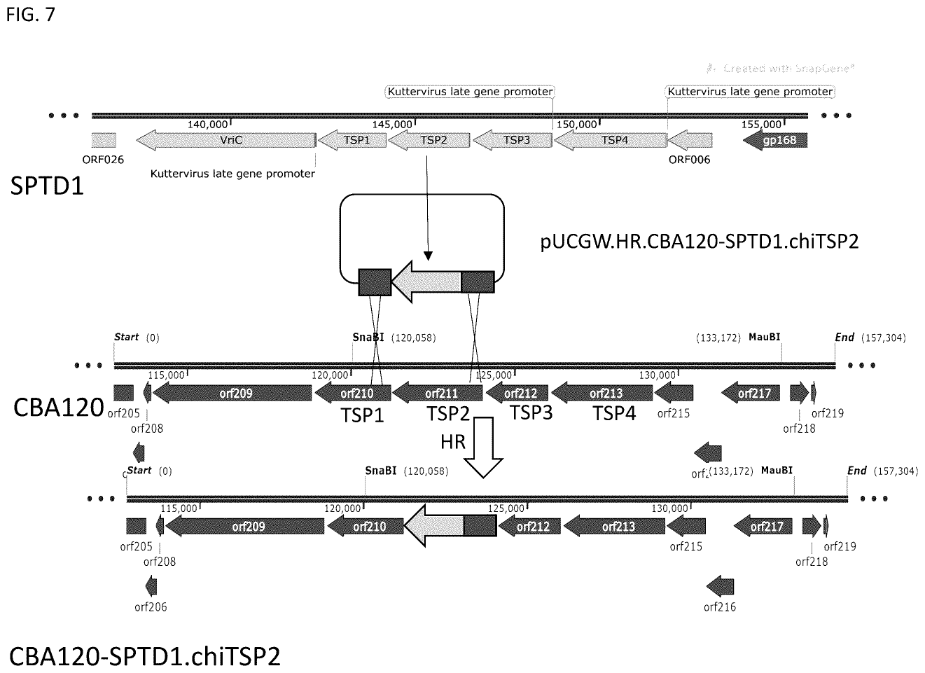

An exemplary synthetic phage was constructed from naturally occurring parent phage CBA120 by replacing CBA120's TSP3 with a recombinant TSP (“CBA120-SPTD1 TSP3”) containing the N-terminal domain from CBA120 TSP3 and the C-terminal domain from phage SPTD1 TSP3. Phage SPTD1 (SEQ ID:1; GenBank Accession No. OP991882) was isolated from sewage samples and sequenced, as discussed in U.S. patent application Ser. No. 16/247,486 filed Jan. 14, 2019, in which SPTD1 is referred to as TSP1. The sequences of CBA120 phage are available at NCBI GenBank under accession number NC_016570. Each of CBA120 and SPTD1 phages has four TSPs: TSP1, TSP2, TSP3 and TSP4 (six copies each). Examples of electron microscopy images of SPTD1 and CBA120 phages are shown in FIG. 6. FIG. 7 schematically illustrates the structure of CBA120 virion with TSP1, TSP2, TSP3, and TSP4 proteins, CBA120 TSPs recognize the following bacteria: TSP1—Salmonella Minnesota; TSP2—E. coli 0157; TSP3—E. coli 077; TSP4—E. coli 078. SPTD1 TSP2 recognizes Citrobacter sedlakii, and SPTD1 TSP3 recognizes Salmonella.

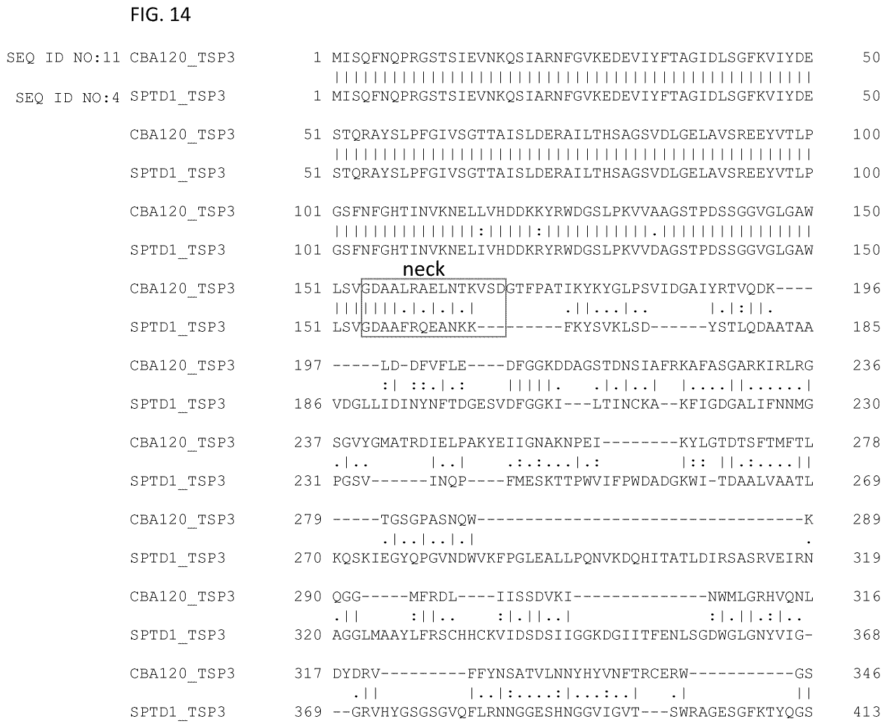

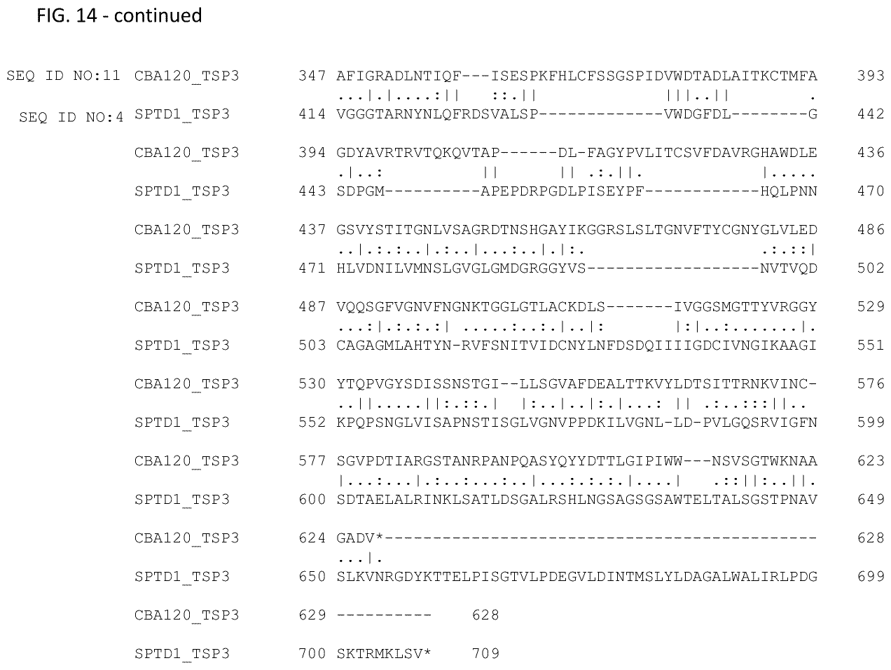

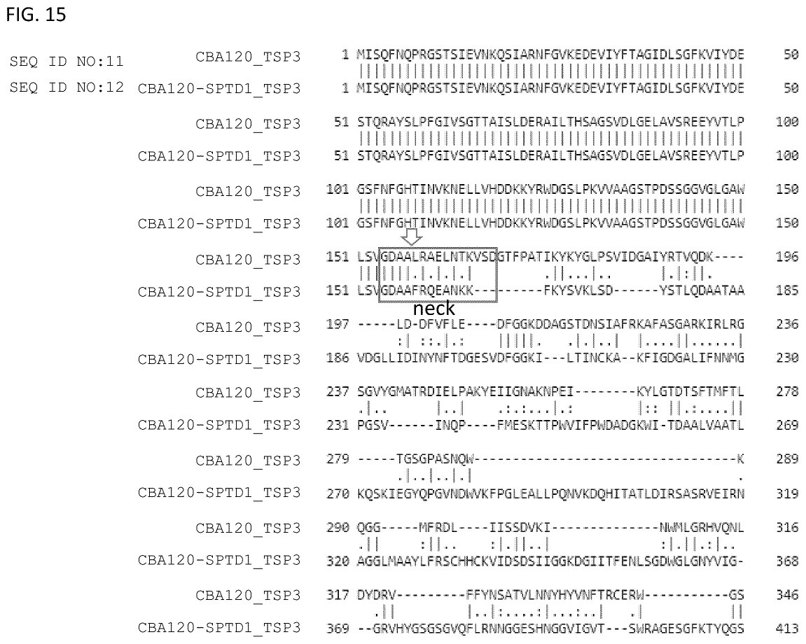

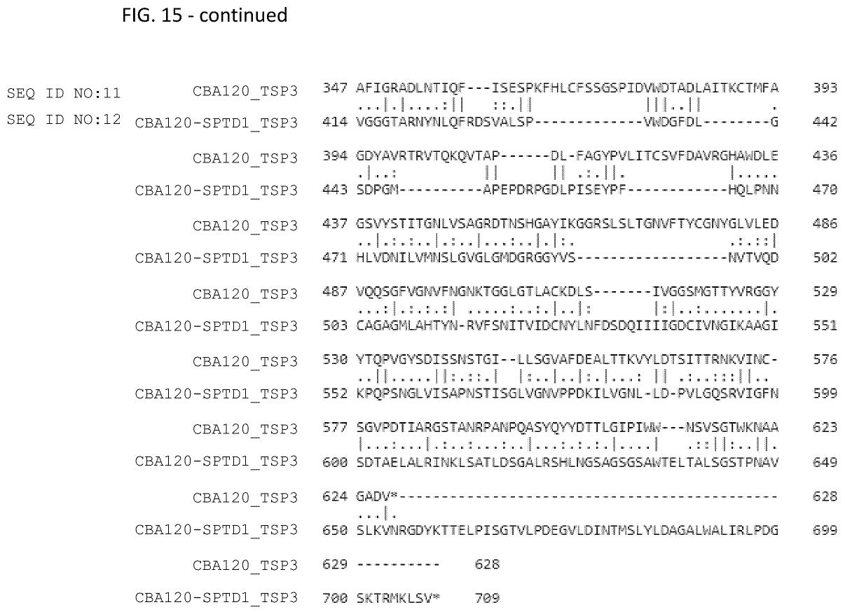

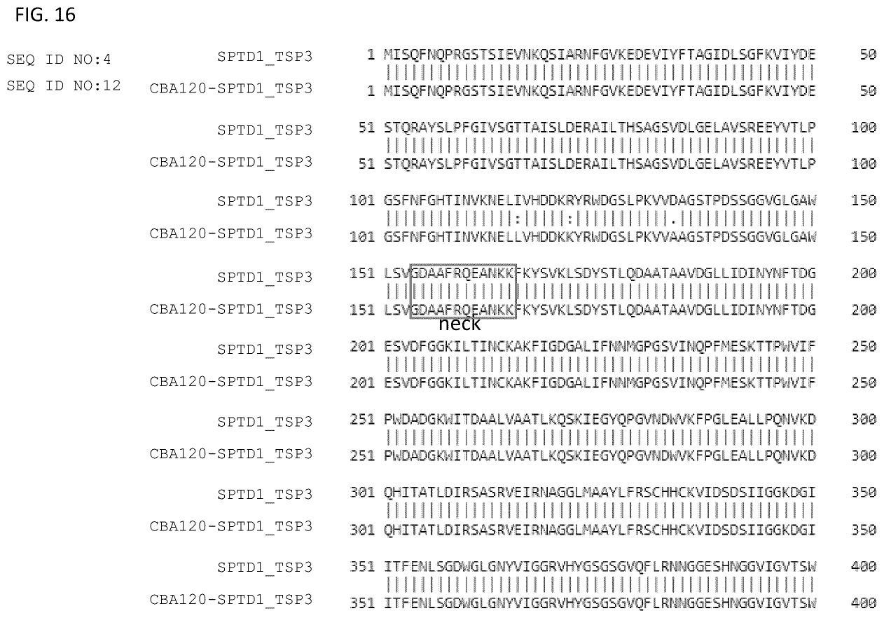

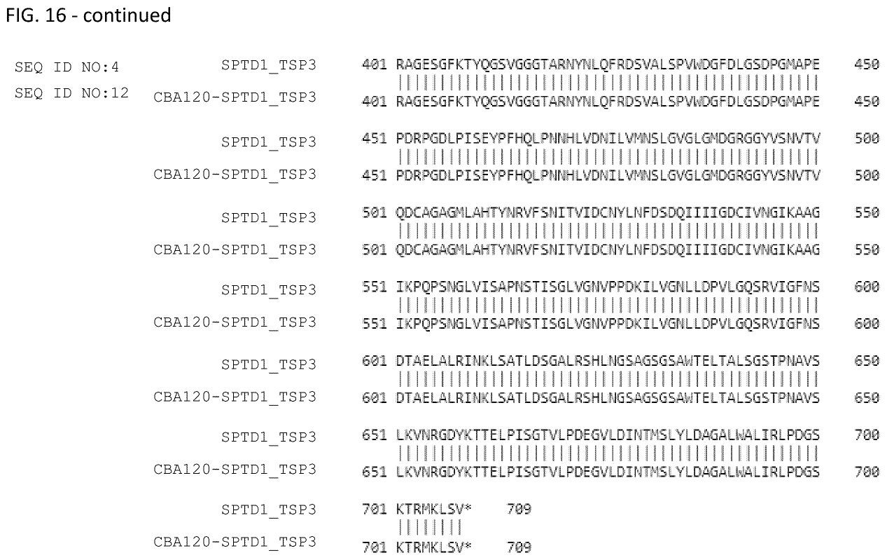

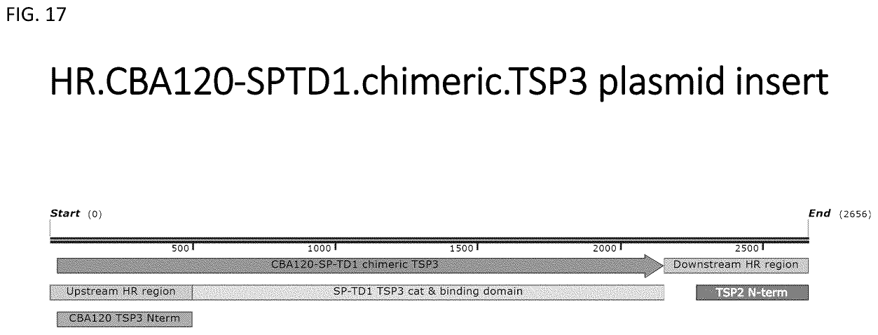

To design the nucleic acid sequence encoding CBA120-SPTD1 TSP3, amino acid sequences of CBA120 TSP3 and SPTD1 TSP3 were analyzed. FIG. 14 is a schematic illustration of the results of the pairwise alignment, performed on EMBOSS Needle, of amino acid sequences of CBA120 TSP3 and SPTD1 TSP3. The “neck” region of both TSPs is indicated with a box. CBA120 TSP3 showed N-terminal homology to SPTD1 TSP3. Additional amino acid sequence analysis showed that Swiss Fold region of CBA120 TSP3 matched N-terminal region of SPTD1 TSP3 as well as C-terminal region of Salmonella phage Det7 TSP3 (which recognizes Salmonella Typhimurium (LT2) ATCC 19585), with the alpha helices being in common between the two proteins. Det7 genomic sequences have GenBank Accession Nos. NC_027119.1 and/or KP797973.1. Det7 TSP3 and SPTD1 TSP3 amino acid sequences showed 95% identity over 90% coverage, serving as potential evidence of horizontal gene transfer. DET After the amino acid sequences of CBA120 TSP3 and SPTD1 TSP3 were analyzed, the site between amino acids 158 and 159 in the “neck” region of each TSP was selected as a joining (“splice”) site N-terminal domain of CBA120 TSP3 and C-terminal domain of SPTD1 TSP3 and. FIG. 15 illustrates pairwise alignment, performed on Emboss Needle, of amino acid sequences of CBA120 TSP3 and recombinant CBA120-SPTD1 TSP3. The joining site between amino acids 158 and 159 in the “neck” region (indicated with a box) of recombinant CBA120-SPTD1 TSP3 is indicated by an arrow. FIG. 16 illustrates pairwise alignment, performed on Emboss Needle, of amino acid sequences of SPTD1 TSP3 and recombinant CBA120-SPTD1 TSP3. The “neck” region is indicated with a box.

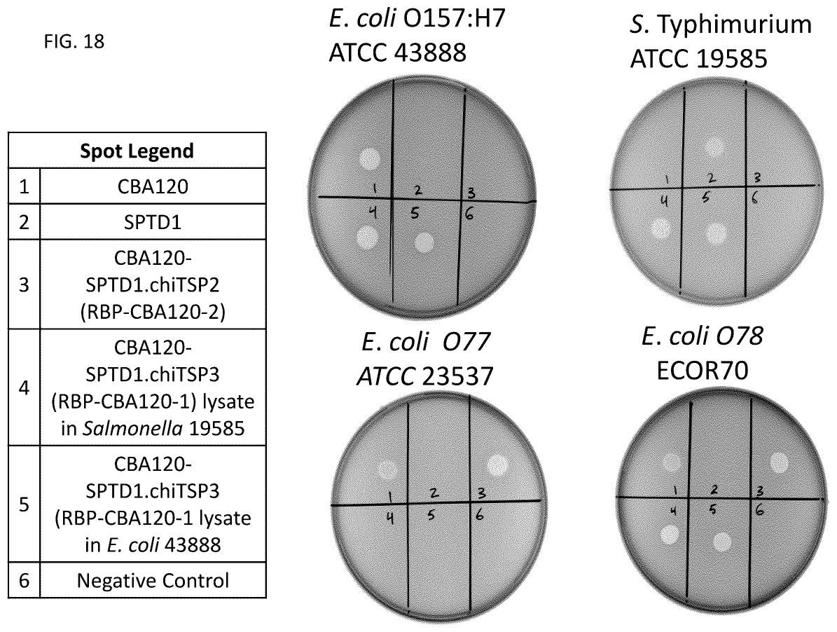

Nucleic acid sequence encoding CBA120-SPTD1 TSP3 was generated by determining where the probable “neck” region was in SPTD1 TSP3 based on homology to CBA120 TSP3, structure predictions with Swiss-Model and Protein Homology/analogY Recognition Engine V 2.0 (Phyre2, available online from Structural Bioinformatics Group, Imperial College, London), determining the corresponding regions in the nucleotide sequence from the amino acid sequence, copying the nucleic acid sequence encoding SPTD1 TSP3 “neck” region to the end of the gene, and replacing the corresponding “neck” to the end of CBA120 TSP3 gene to generate the chimeric open reading frame (ORF) “CBA120-SP-TD1 chimeric TSP3.” The copied section, “SPTD1 TSP3 catalytic & binding domain” is considered the donor DNA sequence. 500 bp of flanking CBA120 genomic sequences on both sides of the sequence to be replaced were on the respective ends of the donor sequence and synthesized in the pUCIDT plasmid used for subsequent HR by Integrated DNA Technologies (IDT). FIG. 18 is a schematic illustration of the HR.CBA120-TSP1.chimeric.TSP3 HR cassette. pUCIDT.HR.CBA120-SPTD1.chimeric.TSP3 plasmid contained nucleic acid sequence encoding CBA120 TSP3 N-terminal domain through the codon encoding amino acid 158, followed by the codon encoding amino acid 159 of SPTD1 TSP3 and the nucleic acid sequence encoding C-terminal domain of SPTD1 TSP3.

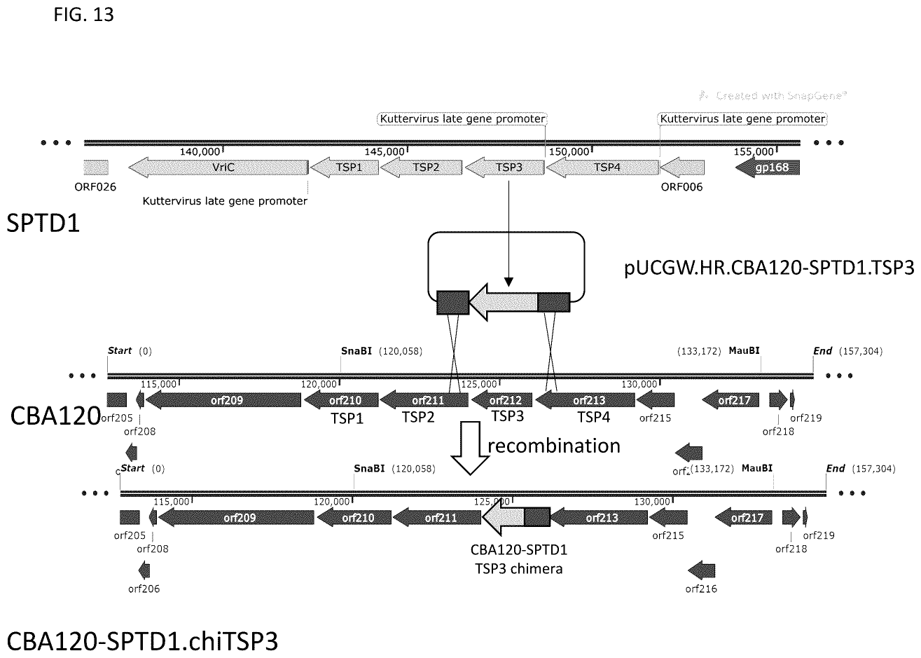

HR was performed as follows to generate a synthetic phage from SPTD1 phage by exchanging SPTD1 TSP3 with a recombinant CBA120-SPTD1 TSP3 containing N-terminal domain from SPTD1 TSP3 and C-terminal domain from CBA120 TSP3. The HR plasmid containing pUCIDT.HR.CBA120-SPTD1.chimeric.TSP3, was transformed into E. coli O157:H7 ATCC 43888 via electroporation with a MicroPulser (Bio-Rad® Laboratories, Inc., Hercules, California, USA) as per manufacturer instructions. Five colonies were grown in 2 ml TSB with 100 μg/ml carbenicillin at 37° C. with 250 rpm shaking until reaching 10⁷ cells/ml. The transformed bacteria, along with an untransformed control were infected at MOI 0.1 of CBA120.NL, a NanoLuc® (Promega Corporation, Madison, Wisconsin, USA) luciferase-expressing recombinant version of CBA120, and incubated for 3 hours at 37° C. with 250 rpm shaking, then harvested and centrifuged at 6800 g for 2 minutes. The supernatant was filtered through a 0.45 μm pore size filter and stored at 4° C. The lysates were serially diluted, and spotted on Petri dishes seeded with either E. coli O157:H7 43888 as a control, or a panel of Salmonella strains (ATCC 19585, 7001, 14028, 27869, and 51158), several of which contain the known target of SPTD1 TSP3, 04 antigen of Salmonella Typhimurium LT2 strains. Individual plaques, which were discernable from the spot assay from Salmonella ATCC 19585 and 14028 were picked and used to inoculate Salmonella LT2 strain ATCC 19585 cultures, which were subsequent prepped as large scale stocks. FIG. 13 is a schematic illustration of the HR process used to generate a synthetic phage from SPTD1 phage by exchanging SPTD1 TSP3 with a recombinant TSP (“CBA120-SPTD1 TSP3”) containing C-terminal domain from SPTD1 TSP3 and N-terminal domain from CBA120 TSP3. The resulting synthetic phage was termed CBA120-SPTD1.chiTSP3 or, alternatively, RBP-CBA120-1.

Plating experiments were performed to test the properties of synthetic CBA120-SPTD1.chiTSP3 (alternatively named RBP-CBA120-1) phage. The results of exemplary plating experiments are illustrated in FIG. 18, and are also summarized in Tables 1 and 2. In comparison to CBA120, synthetic phage CBA120-SPTD1.chiTSP3 lost the ability to infect E. coli 077 and gained the ability to infect S. Typhimurium.

### Example 2: Construction and Testing of Synthetic Phage with Recombinant TSP2

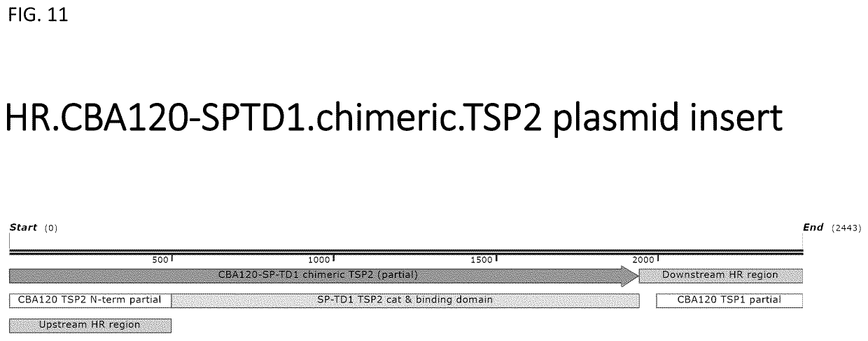

An exemplary synthetic phage was constructed from naturally occurring parent phage CBA120 by replacing CBA120 TSP2 with recombinant TSP (“CBA120-SPTD1 TSP2”) containing N-terminal domain from CBA120 TSP2 and C-terminal domain from phage SPTD1 TSP2. Nucleic acid sequence encoding CBA120-SPTD1 TSP2 was produced, and a synthetic phage termed CBA120-SPTD1.chiTSP2 (alternatively named RBP-CBA120-2) was constructed using the procedures substantially similar to those described in Example 1. FIG. 11 is a schematic illustration of HR.CBA120-SPTD1.chimeric.TSP2 HR cassette. This HR cassette contained nucleic acid sequence encoding CBA120 TSP2 N-terminal domain through the codon encoding amino acid 244, followed by the codon encoding amino acid 245 of SPTD1 TSP2, and the nucleic acid sequence encoding C-terminal domain of SPTD1 TSP2. FIG. 7 is a schematic illustration of HR process used to generate a synthetic phage from CBA120 phage by exchanging SPTD1 TSP2 with a recombinant TSP (“CBA120-SPTD1 TSP2”) containing N-terminal domain from CBA120 TSP2 and C-terminal domain from SPTD1 TSP2. The resulting synthetic phage was termed CBA120-SPTD1.chiTSP2 or, alternatively, RBP-CBA-120-2.

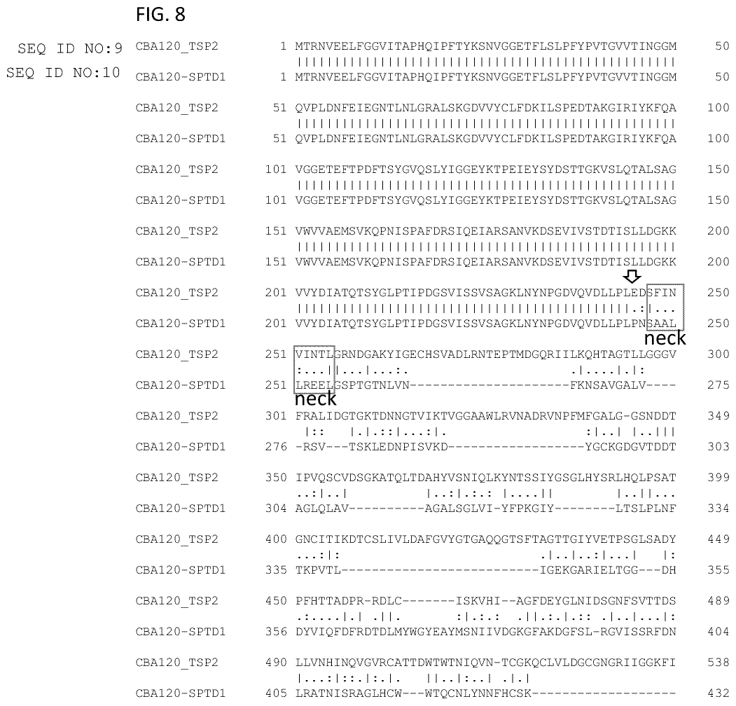

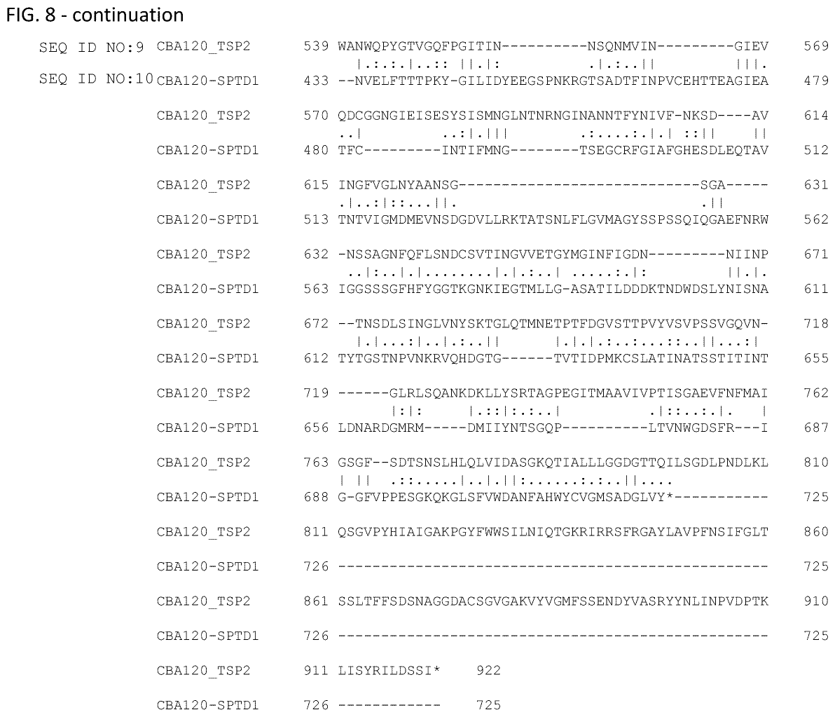

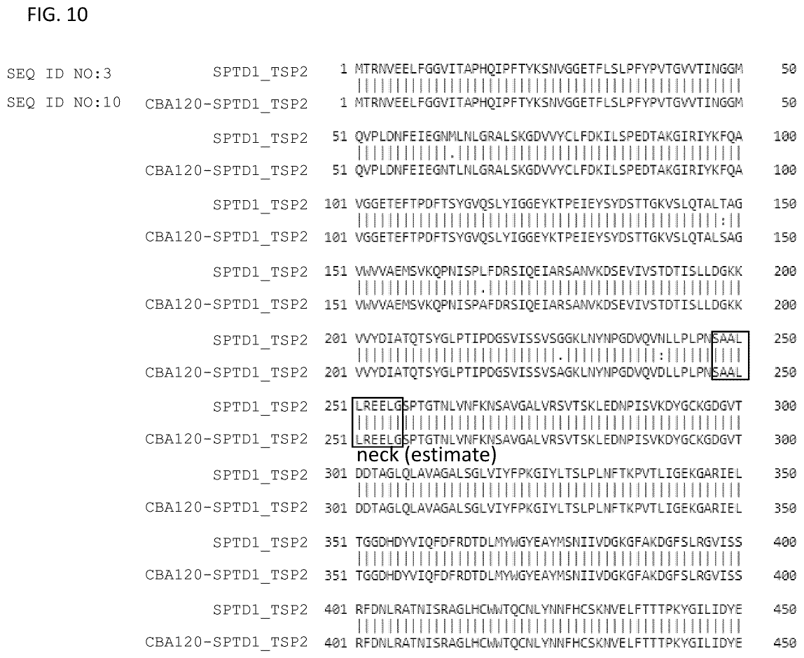

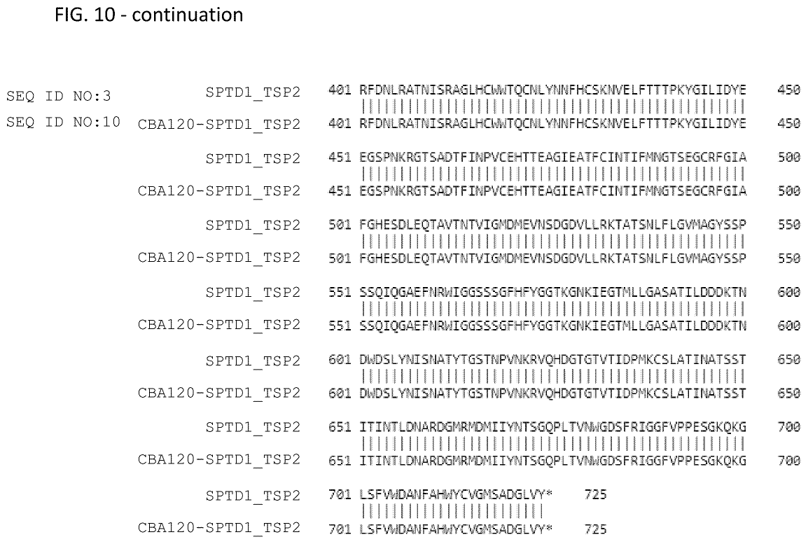

To design the nucleic acid sequence encoding CBA120-SPTD1 TSP2, amino acid sequences of CBA120 TSP2 and SPTD1 TSP2 were analyzed. FIG. 8 is a schematic illustration of the results of the pairwise alignment of amino acid sequences of CBA120 TSP2 and SPTD1 TSP2 performed on Emboss Needle. The “neck” region of both TSPs is indicated with a box. As illustrated in FIG. 8, the amino acid sequences are identical until two amino acids upstream of neck (as indicated by an arrow). After the amino acid sequences of CBA120 TSP2 and SPTD1 TSP2 were analyzed, the site between amino acids 244 and 245 of each TSP was selected as a joining (“splice”) site between C-terminal domain of SPTD1 TSP2 and N-terminal domain of CBA120 TSP2. FIG. 9 illustrates, in the left panel, pairwise alignment, performed on Emboss Needle, of amino acid sequences of CBA120 TSP2 and recombinant CBA120-SPTD1 TSP2 in the region of the joining site between the two sequences. The joining site between amino acids 244 and 245 of recombinant CBA120-SPTD1 TSP2 is indicated by an arrow. As illustrated in the right panel of FIG. 9, the structure of recombinant CBA120-SPTD1 TSP2 protein contains three parts: the N-terminal region that binds to the baseplate of bacteriophage virion via TSP4 (TSP2 binds to the XD2 domain in the N-terminal region of TSP4, while TSP4 binds to the baseplate via XD1 domain), the “neck” (“hinge”) region and the C-terminal region that binds to a bacterial receptor. FIG. 10 illustrates pairwise alignment, performed on Emboss Needle, of amino acid sequences of SPTD1 TSP2 and recombinant CBA120-SPTD1 TSP2. The estimated “neck” region is indicated with a box. Recombinant CBA120-SPTD1 TSP2 includes complete neck alpha helix from SPTD1 TSP2.

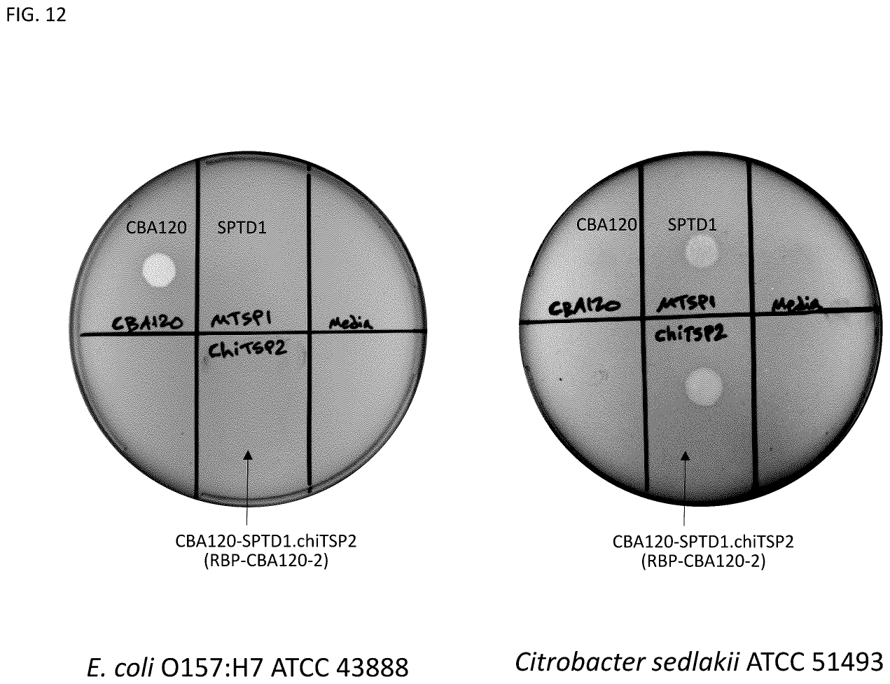

Plating experiments were performed to test the properties of synthetic CBA120-SPTD1.chiTSP2 (RBP-CBA-120-2) phage. The results of exemplary plating experiments are illustrated in FIG. 12 and FIG. 18, and are also summarized in Tables 1 and 2. In comparison to CBA120, synthetic phage CBA120-SPTD1.chiTSP2 lost the ability to infect E. coli 0157 and gained the ability to infect Citrobacter sedlakii.

### Example 3: Construction and Testing of Synthetic Phage with Recombinant TSP4

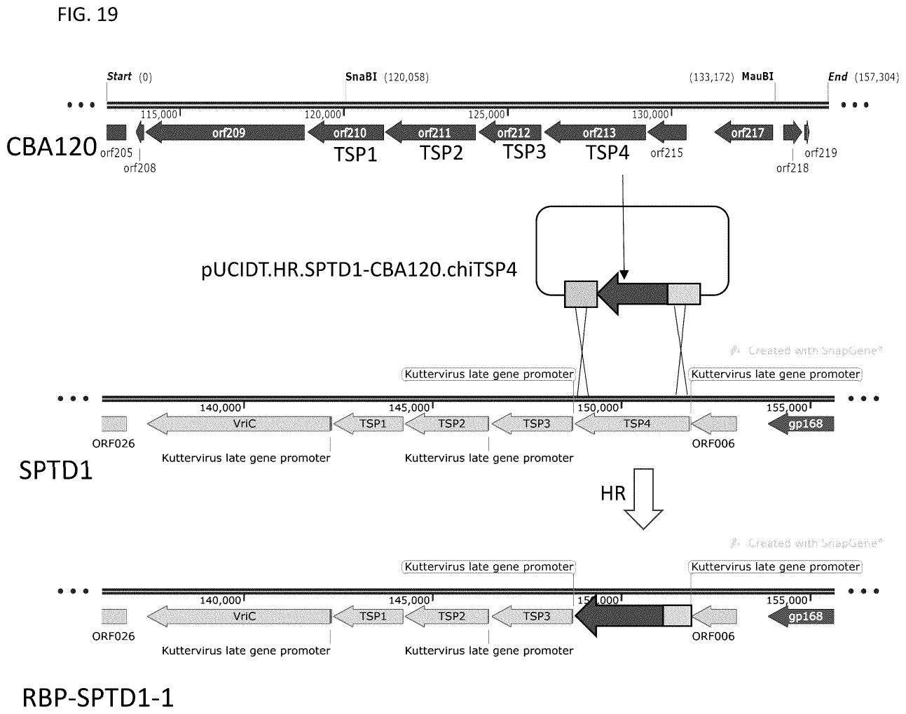

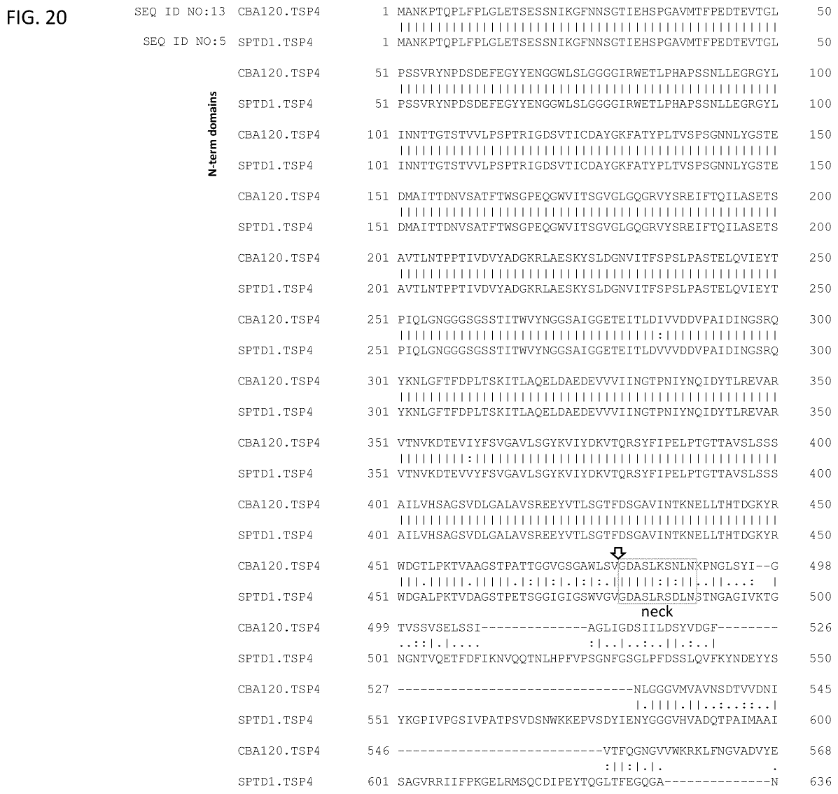

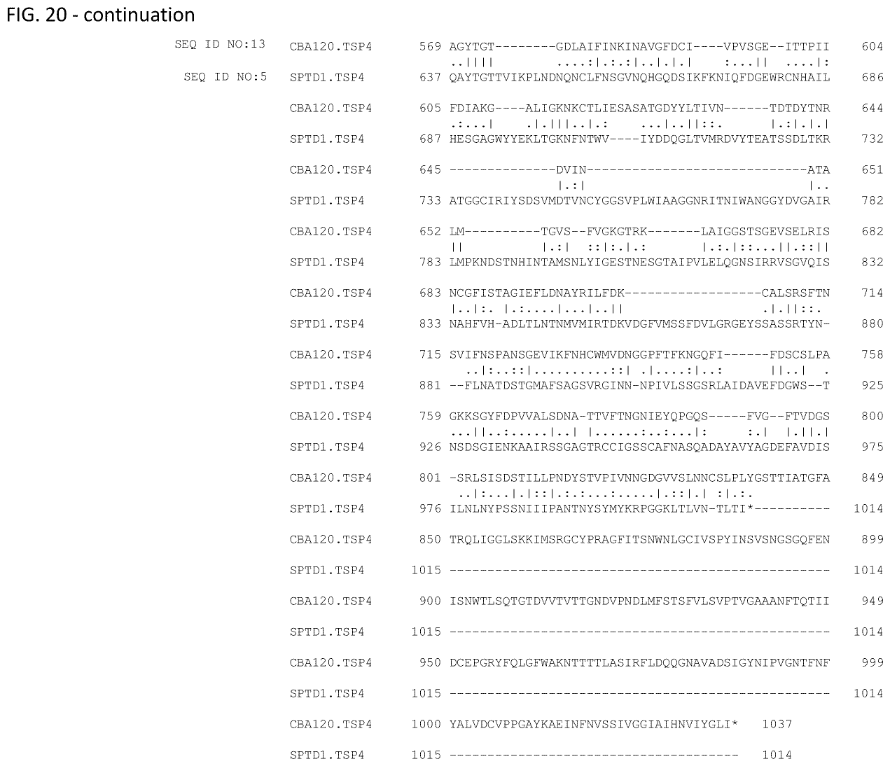

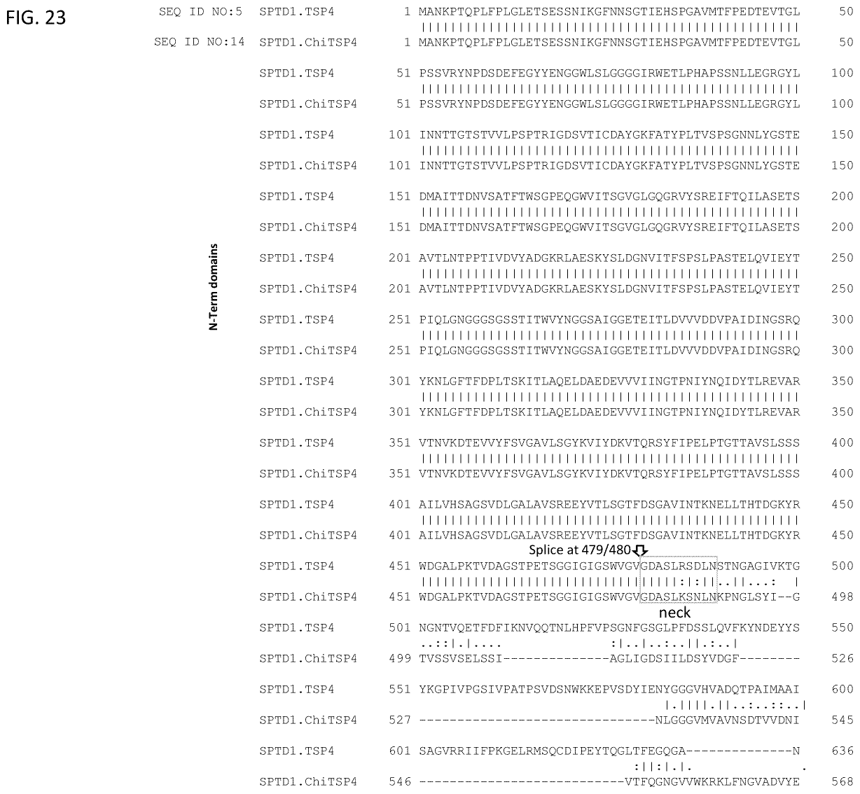

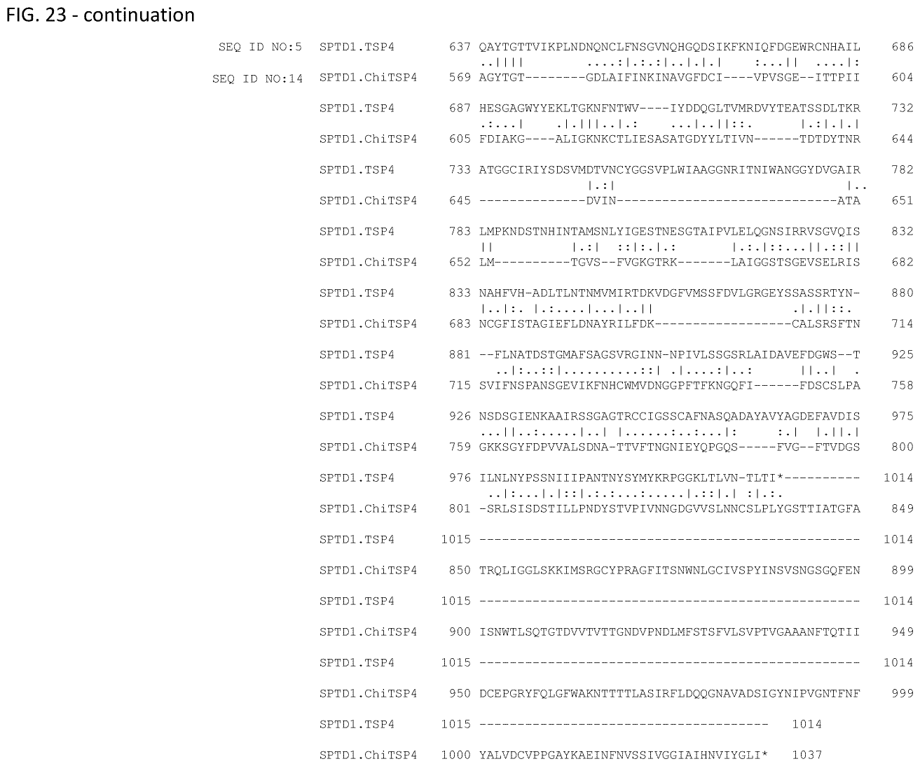

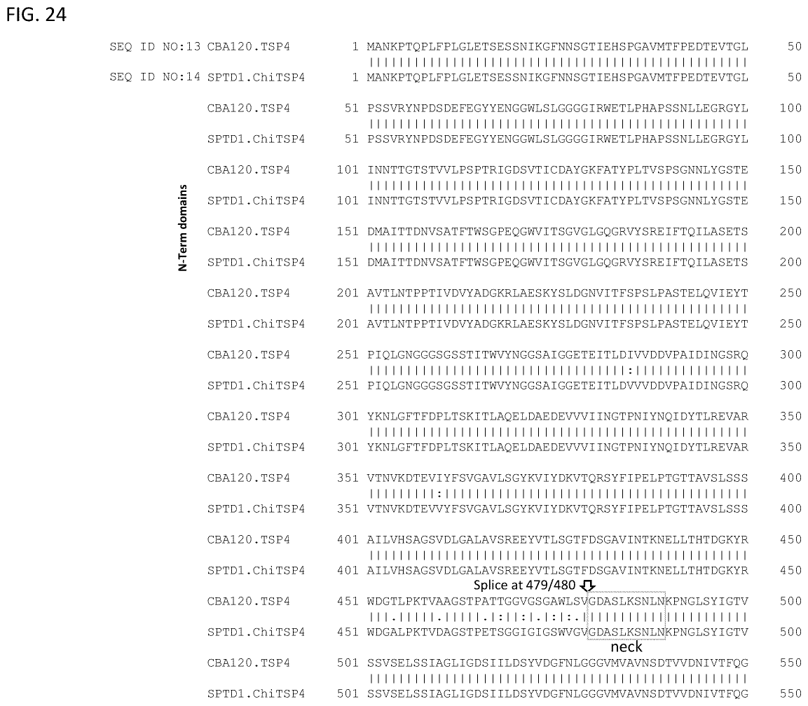

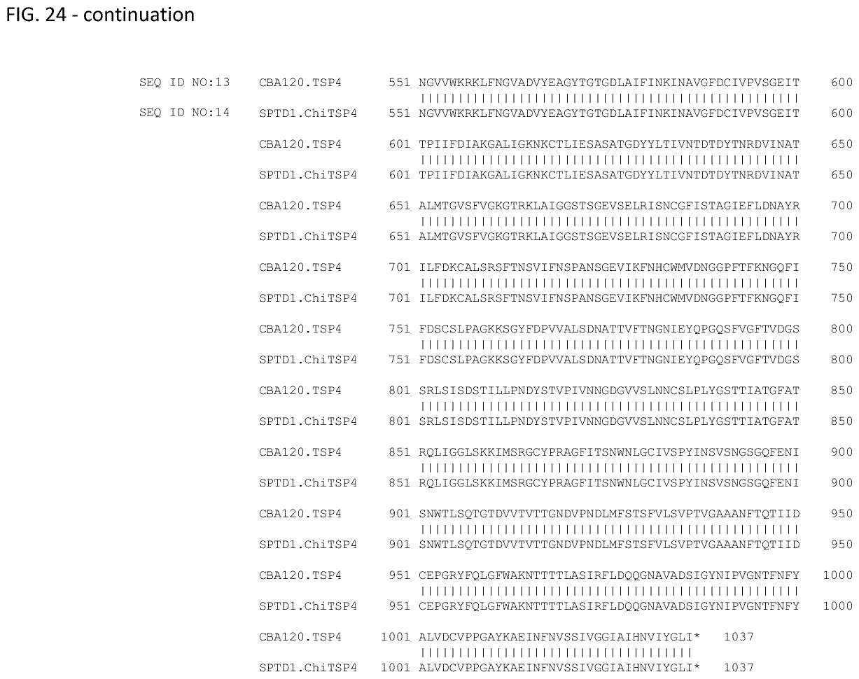

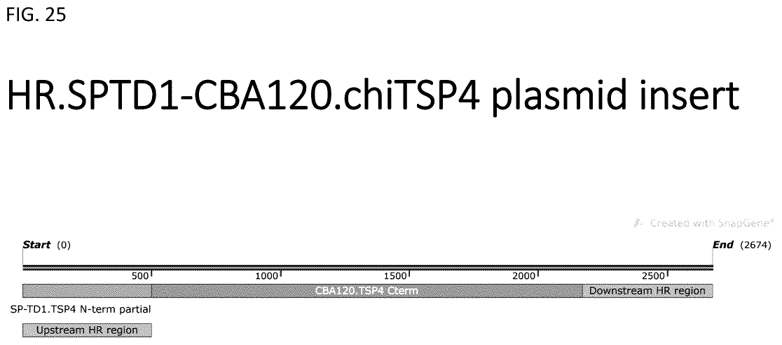

An exemplary synthetic phage was constructed from naturally occurring parent phage SPTD1 by replacing SPTD1 TSP4 with recombinant TSP (“SPTD1-CBA120 TSP4”) containing N-terminal domain from SPTD1 TSP4 and C-terminal domain from CBA120 TSP4. Nucleic acid sequence encoding SPTD1-CBA120 TSP4 was produced and synthetic phage termed SPTD1-CBA120.chiTSP4 (alternatively named RBP-SPTD1-1) was constructed using the procedures substantially similar to those described in Example 1. To design the nucleic acid sequence encoding SPTD1-CBA120 TSP4, amino acid sequences of CBA120 TSP4 and SPTD1 TSP4 were analyzed. FIG. 21 is a schematic illustration of the results of the pairwise alignment of amino acid sequences of CBA120 TSP4 and SPTD1 TSP4 performed on Emboss Needle. The “neck” region of SPTD1 TSP4 is indicated. After the amino acid sequences of CBA120 TSP4 and SPTD1 TSP4 were analyzed, the site between amino acids 479 and 480 of each TSP was selected as a joining (“splice”) site between N-terminal domain of SPTD1 TSP4 and C-terminal domain of CBA120 TSP4. FIG. 23 illustrates pairwise alignment, performed on Emboss Needle, of amino acid sequences of SP-TD1 TSP4 and recombinant SPTD1-CBA120 TSP4. The estimated “neck” region is indicated with a box. FIG. 21 is a schematic illustration of the structure CBA120 TSP4 protein. FIG. 22 is a schematic illustration of the hypothetical structure of recombinant SPTD1-CBA120 TSP4 protein. FIG. 23 illustrates pairwise alignment, performed on Emboss Needle, of amino acid sequences of SPTD1 TSP4 and recombinant SPTD1-CBA120 TSP4. The joining site between amino acids 479 of SPTD1 TSP4 and amino acid 480 of CBA120 TSP4 is indicated by an arrow. The “neck” region (based on published TSP structures) is indicated by a box. FIG. 24 illustrates pairwise alignment, performed on Emboss Needle, of amino acid sequences of CBA120 TSP4 and recombinant SPTD1-CBA12 TSP4. The joining site between amino acid 479 of SPTD1 and amino acid 480 of recombinant SPTD1-CBA120-TSP4 is indicated by an arrow. The neck region (based on published TSP structures) is indicated by a box. FIG. 19 is a schematic illustration of the HR process used to generate synthetic phage SPTD1-CBA120.chiTSP4 (alternatively named RBP-SPTD1-1) from SPTD1 by exchanging SPTD1 TSP4 with a recombinant TSP4 (“SPTD1-CBA120 TSP4”) containing N-terminal domain from SPTD1 TSP4 and C-terminal domain from CBA120 TSP4. The HR cassette contained nucleic acid sequence encoding SPTD1 TSP4 N-terminal domain through the codon encoding amino acid 479, followed by the codon encoding amino acid 480 of CBA120 TSP4 and the nucleic acid sequence encoding C-terminal domain of CBA120 TSP4. FIG. 25 is a schematic illustration of the HR.SPTD1-CBA120chiTSP4 plasmid insert.

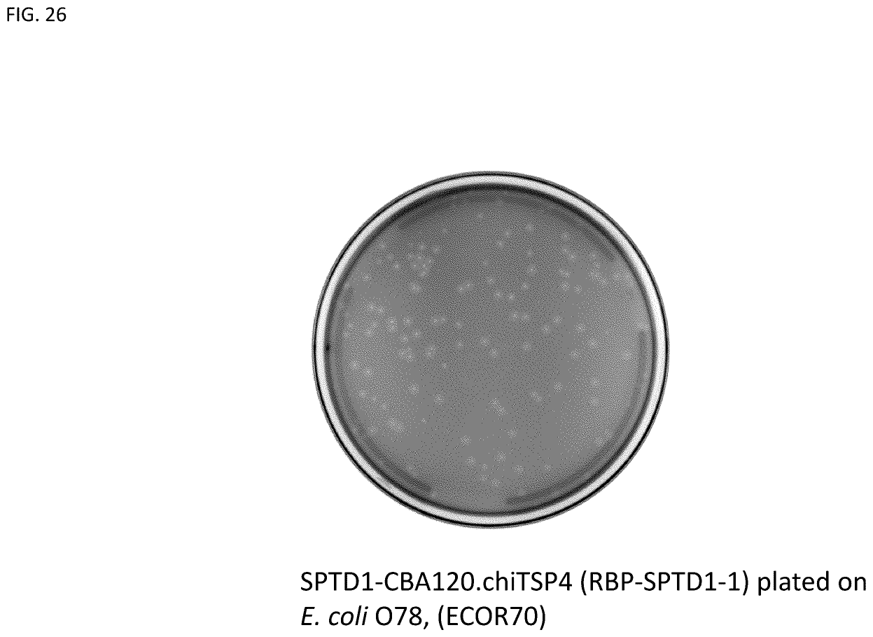

Plating experiments were performed to test the ability of synthetic SPTD1-CBA120.chiTSP4 (RBP-SPTD1-1) phage to infect E. coli 078. FIG. 26 is a photographic image of the plate culture illustrating the results of the plating of synthetic bacteriophage SPTD1-CBA120.chiTSP4 (RBP-SPTD1-1) on E. coli 078.

### Example 4: Construction and Testing of Synthetic Phage with Recombinant TSP1

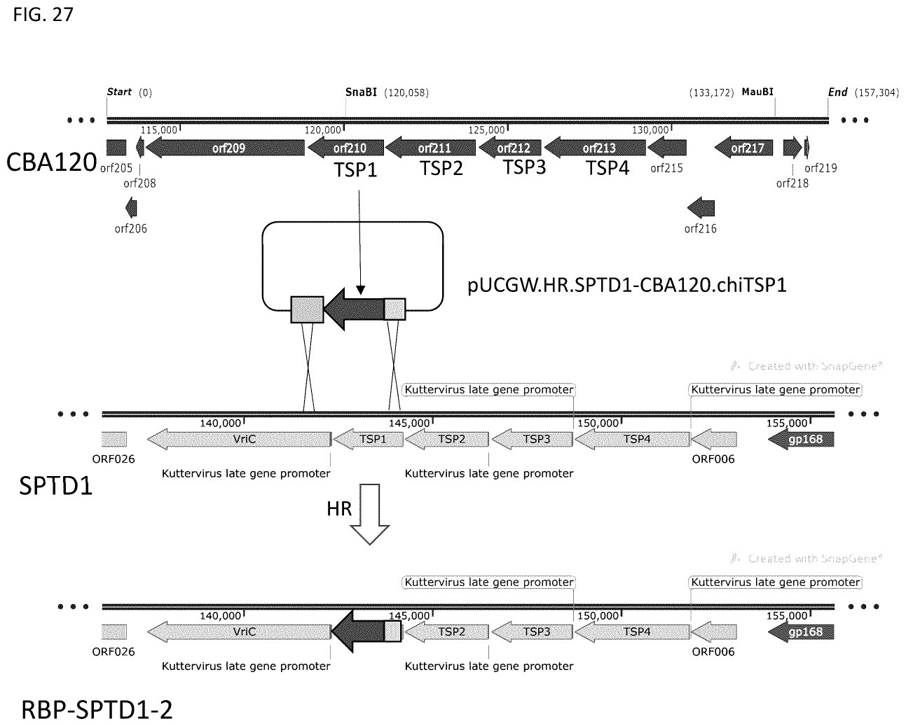

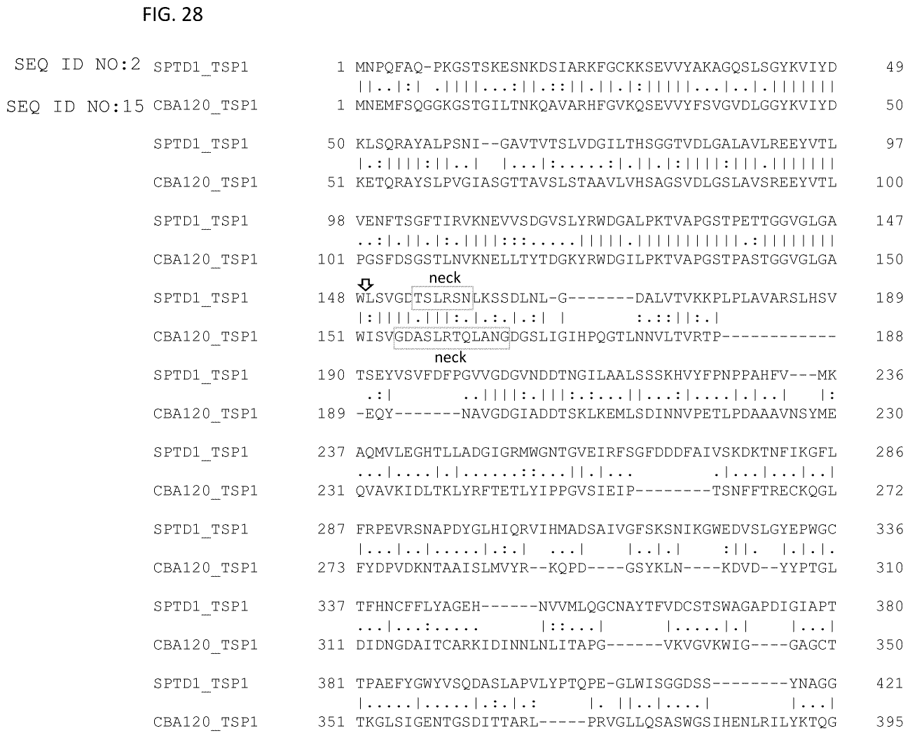

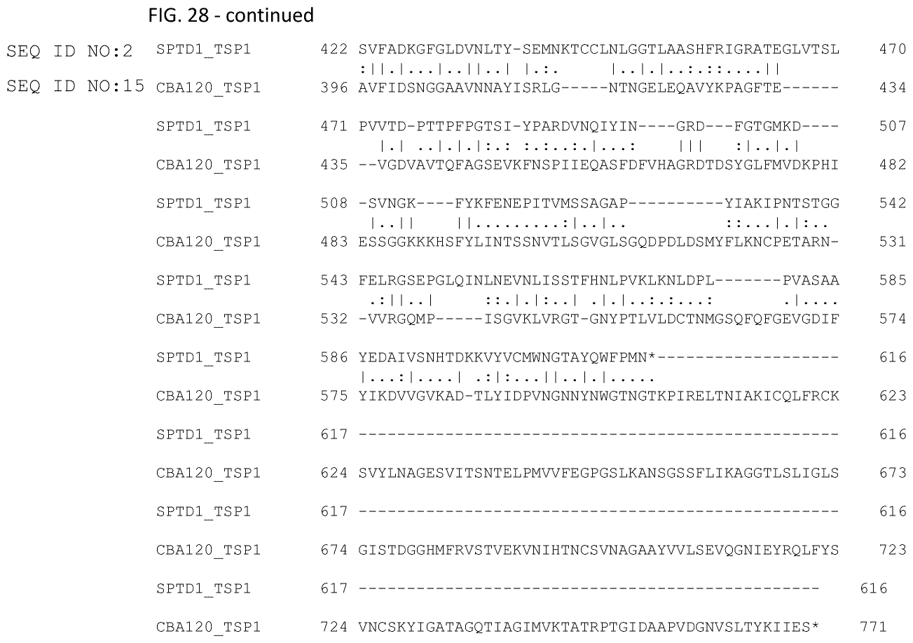

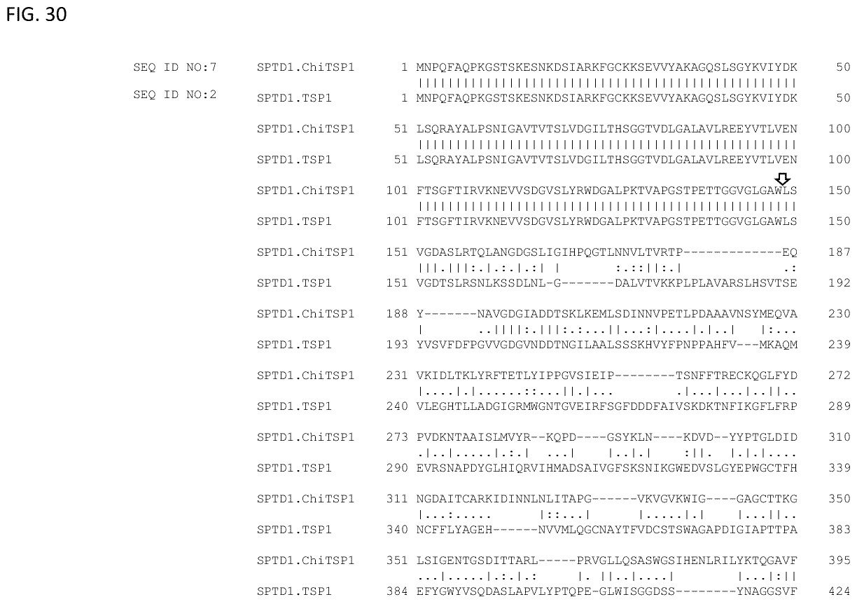

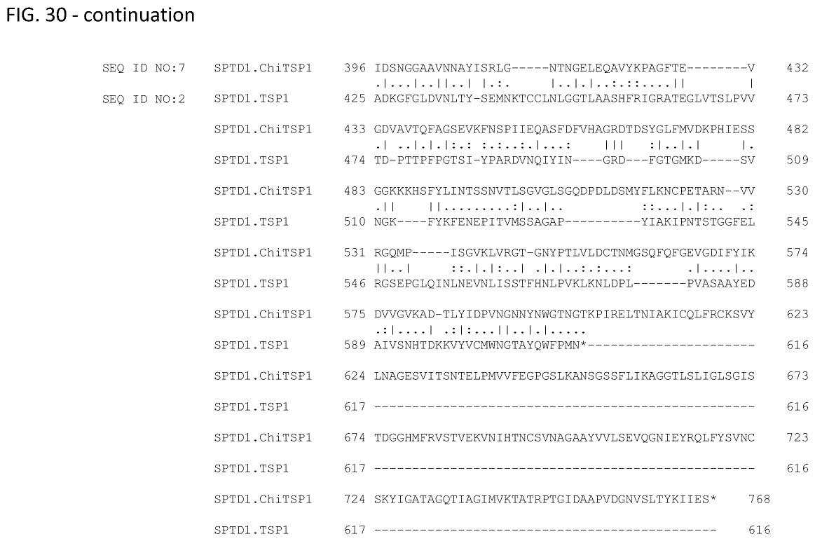

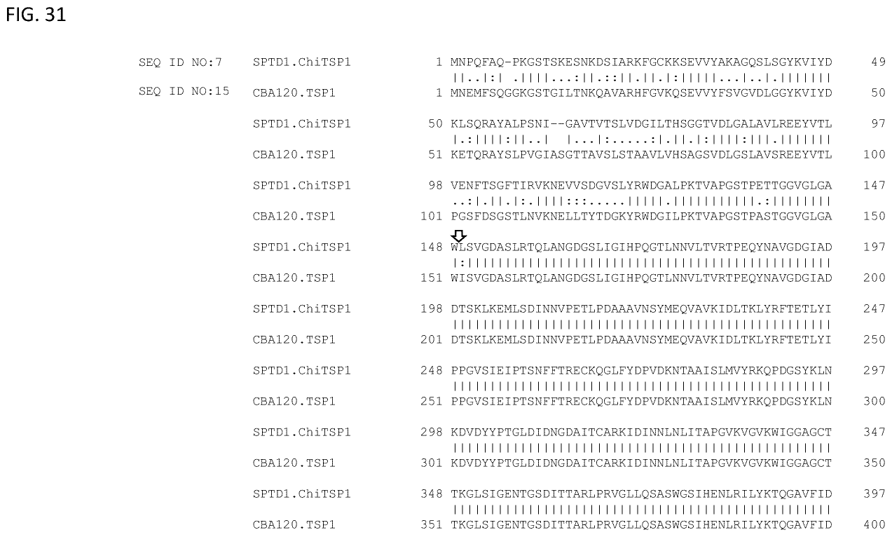

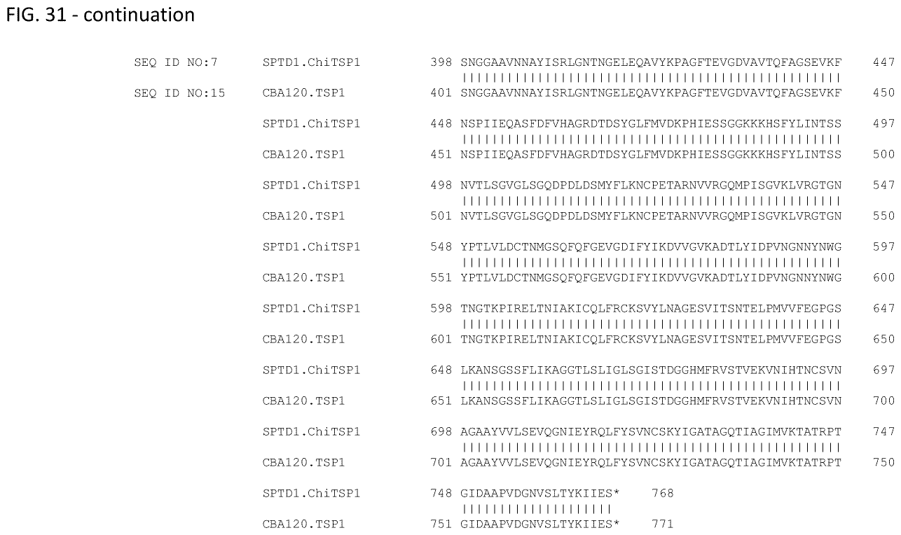

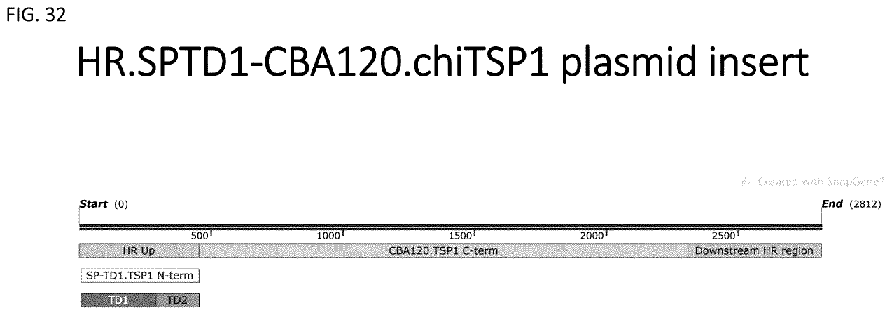

An exemplary synthetic phage was constructed from naturally occurring parent phage SPTD1 by replacing SPTD1 TSP1 with recombinant TSP1 (“SPTD1-CBA120 TSP1”) containing N-terminal domain from SPTD1 TSP1 and C-terminal domain from phage CBA120 TSP1. Nucleic acid sequence encoding SPTD1-CBA120 TSP1 was produced and synthetic phage termed SPTD1-CBA120.chiTSP1 (alternatively named RBP-SPTD1-2) was constructed using the procedures substantially similar to those described in Example 1. FIG. 27 is a schematic illustration of homologous recombination process used to generate synthetic phage SPTD1-CBA120.chiTSP1 (RBP-SPTD1-2) from SPTD1 by exchanging SPTD1 TSP1 with the recombinant TSP1 (“SPTD1-CBA120 TSP1”) containing N-terminal domain from SPTD1 TSP1 and C-terminal domain from CBA120 TSP1. FIG. 28 illustrates pairwise alignment, performed on Emboss Needle, of amino acid sequences of CBA120 TSP1 and SPTD1 TSP1. The arrow indicates the joining site between amino acid 149 of SPTD1 TSP1 and amino acid 152 of CBA120 TSP1. The “neck” region (based on published TSP structures) is indicated by a box. FIG. 29 is a schematic illustration of the structure of CBA120 TSP1 protein. FIG. 30 illustrates pairwise alignment, performed on Emboss Needle, of amino acid sequences of SPTD1 TSP1 and SPTD1-CBA120 TSP1. The joining site between amino acid SPTD1 TSP1 and amino acid 149 of CBA120 TSP1 is marked with an arrow. FIG. 31 illustrates pairwise alignment, performed on Emboss Needle, of amino acid sequences of CBA120 TSP1 and recombinant SPTD1-CBA120 TSP1. The joining site between amino acid SPTD1 TSP1 and amino acid 149 of CBA120 TSP1 is marked with an arrow. FIG. 32 is a schematic illustration of HR.SPTD1-CBA120chiTSP1 plasmid insert. Upstream homologous recombination region is SPTD1 TSP1 N-terminal region, which contains the TD1 and TD2 domains.

### Example 5

Construction and Testing of Synthetic Phage with Recombinant TSP1 and TSP2

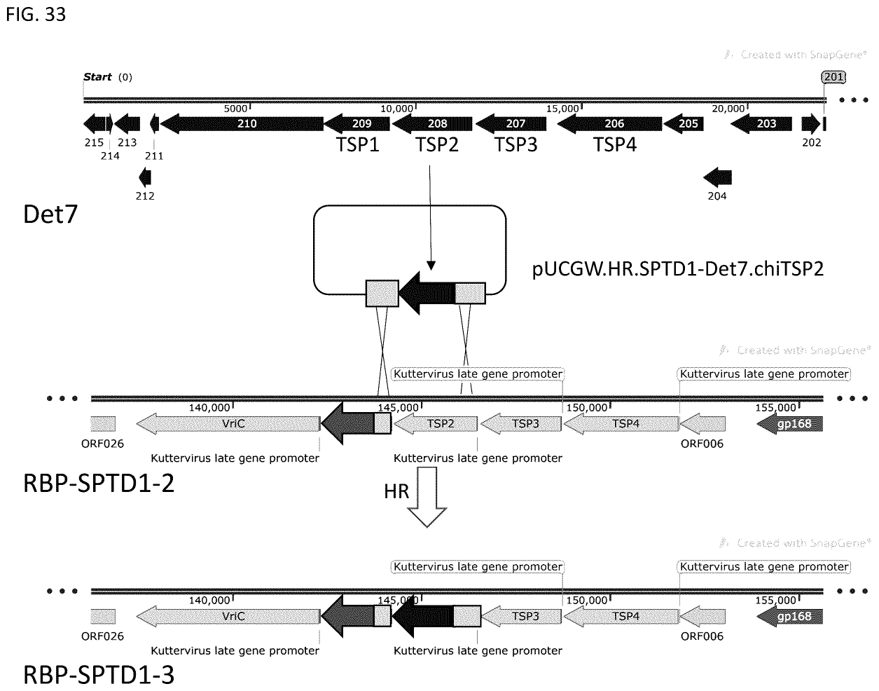

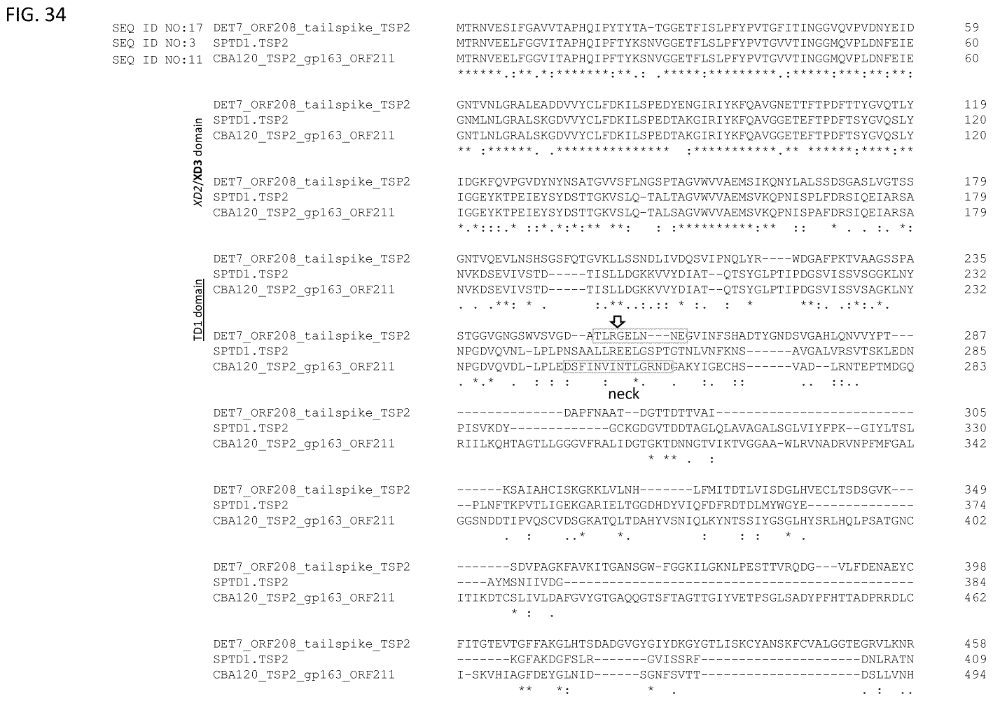

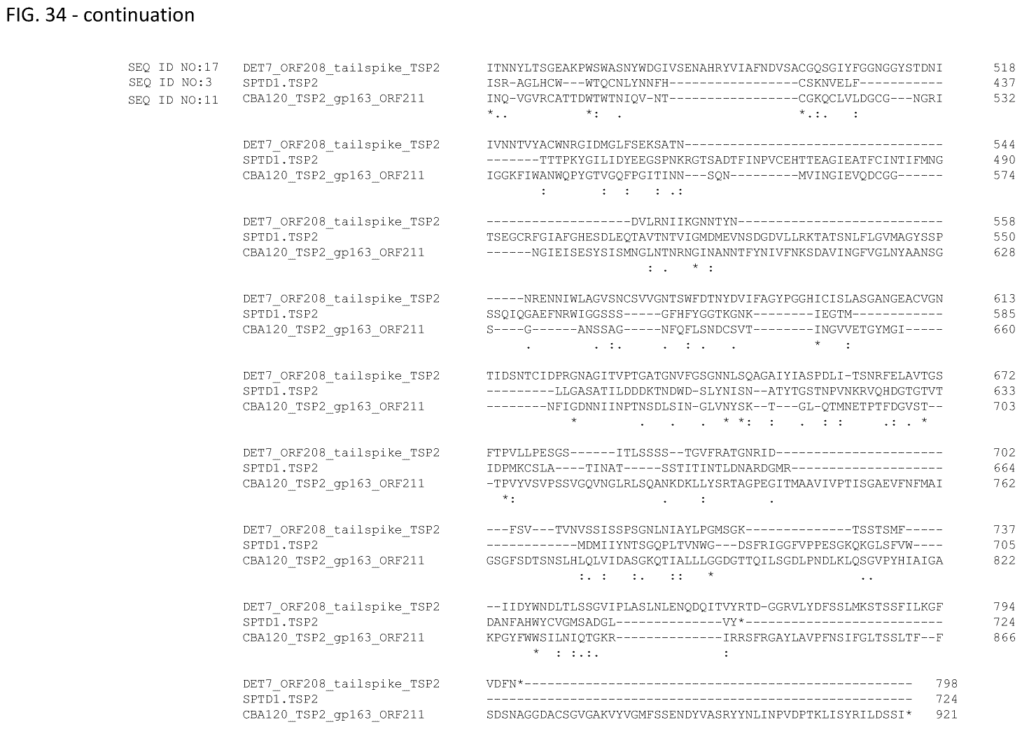

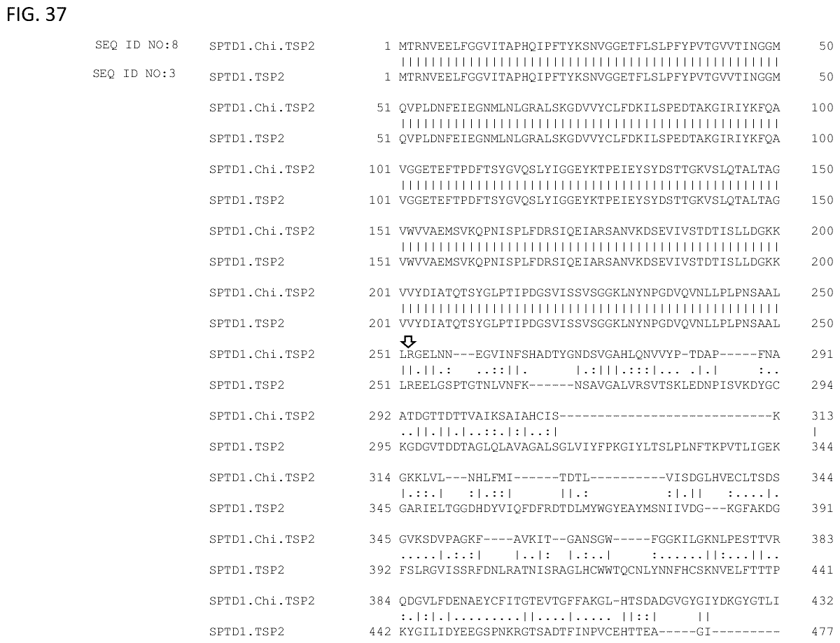

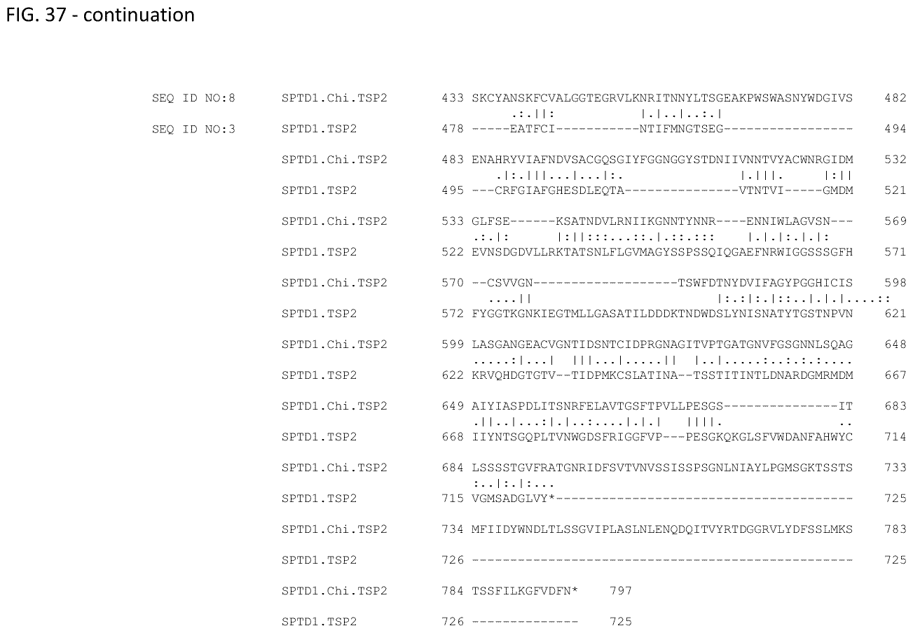

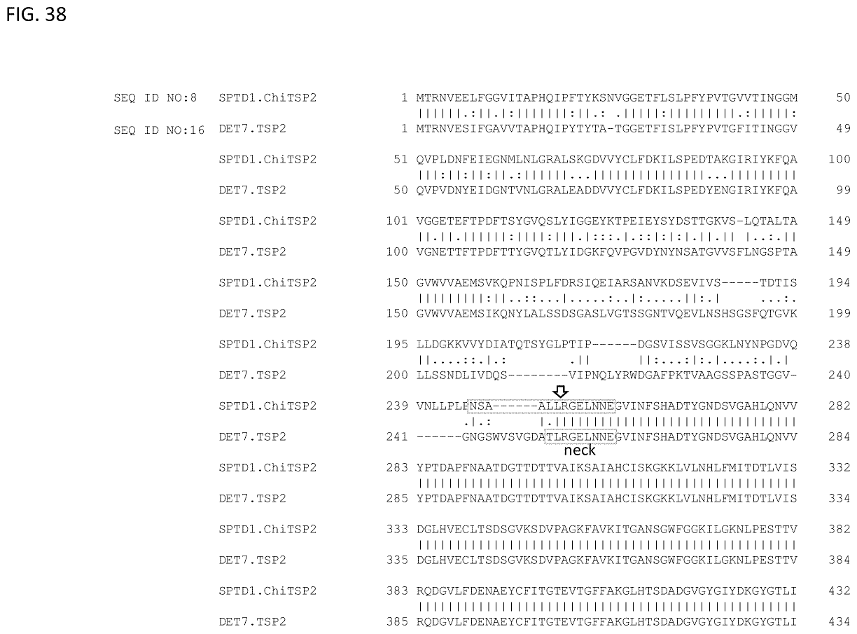

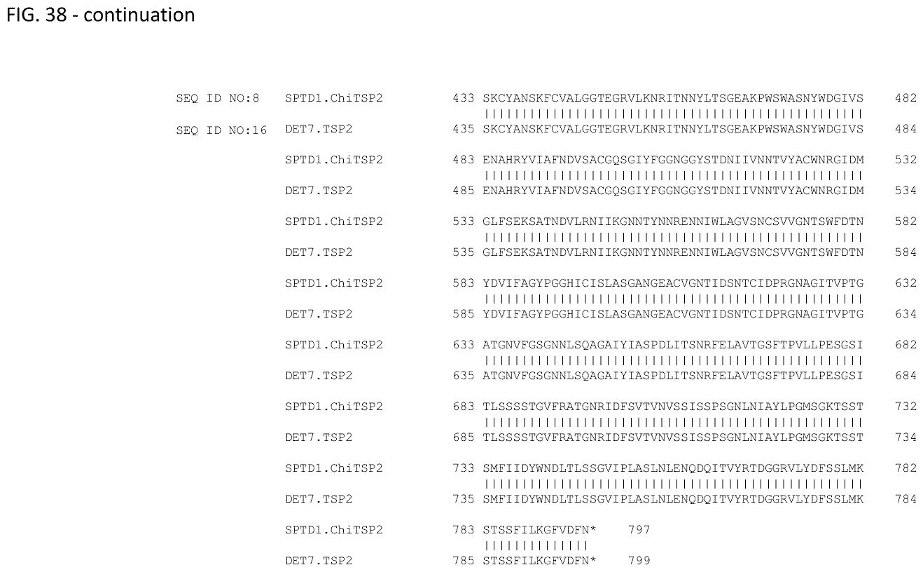

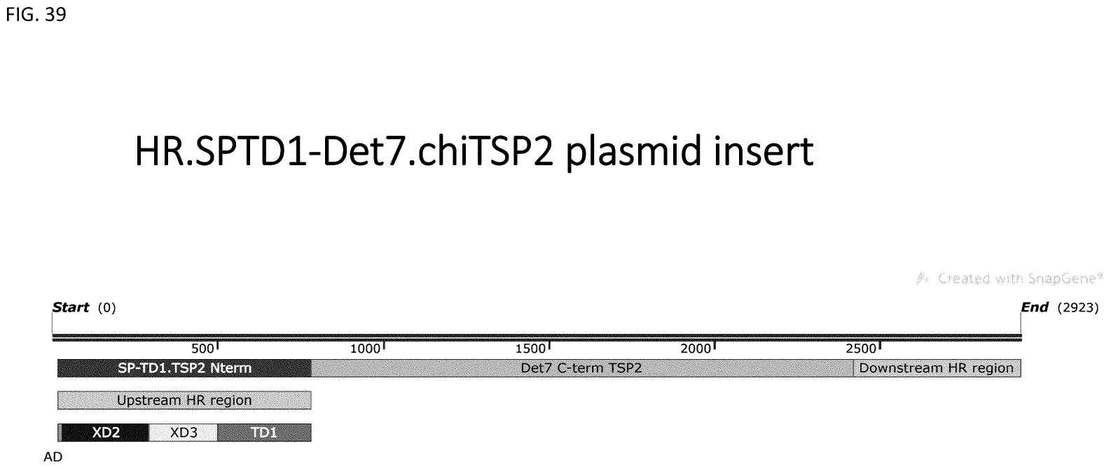

An exemplary synthetic phage was constructed from synthetic parent phage SPTD1-CBA120.chiTSP1 (RBP-SPTD1-2), discussed in Example 4, by replacing its TSP2 with a recombinant TSP 2 (“SPTD1-Det7 TSP2”) containing N-terminal domain from SPTD1 TSP2 and C-terminal domain from Det7 TSP2. Nucleic acid sequence encoding SPTD1-Det7 TSP2 was produced, and synthetic phage termed SPTD1-CBA120.chiTSP1-Det7.chiTSP2 (alternatively named RBP-SPTD1-3) was constructed using the procedures substantially similar to those described in in the previous examples. FIG. 33 is a schematic illustration of the homologous recombination process used to generate synthetic phage SPTD1-CBA120.chiTSP1-Det7.chiTSP2 (alternatively named RBP-SPTD1-3) from SPTD1-CBA120.chiTSP1 (RBP-SPTD1-2) by exchanging TSP2 of SPTD1-CBA120.chiTSP1 (RBP-SPTD1-2) with a recombinant TSP2 (“SPTD1-Det7 TSP2”) containing N-terminal domain from SPTD1 TSP2 and C-terminal domain from Det7 TSP2. FIG. 34 illustrates pairwise alignment, performed on Emboss Needle, of amino acid sequences of Det 7 TSP2, CBA120 TSP2 and SPTD1 TSP2. The arrow indicates the joining site between amino acid 252 of SPTD1 TSP2 and amino acid 256 of Det7 TSP2. The “neck” region based on published TSP structures is indicated by a box. FIG. 35 is a schematic illustration of the structure of CBA120 TSP2 protein. FIG. 36 is a schematic illustration of the predicted structure of a recombinant TSP containing N-terminus of SPTD1 TSP2 and C-terminus of Det7 TSP 2. FIG. 37 illustrates pairwise alignment, performed on Emboss Needle, of amino acid sequences of chimeric SPTD1-Det7 TSP2 (SPTD1.Chi.TSP2) and SPTD1 TSP2. The arrow indicates the joining site between amino acid 252 of SPTD1 TSP2 and amino acid 256 of Det7 TSP2. FIG. 38 illustrates pairwise alignment, performed on Emboss Needle, of amino acid sequences of recombinant SPTD1-Det7 TSP2 and Det7 TSP2. The arrow indicates the joining site between amino acid 252 of SPTD1 TSP2 and amino acid 256 of Det7 TSP2. FIG. 39 is a schematic illustration of HR.SPTD1-CBA120chiTSP2 plasmid insert. Upstream homologous recombination region is SPTD1 TSP2 N-terminal region, which contains attachment domain (AD), XD2, XD3, and TD1 domains.

### Example 6: Testing of Synthetic Phages with Recombinant TSPs

Testing of synthetic phages with recombinant TSPs by spotting a suspension each phage on the plated bacterial strain. The appearance of clear plaques indicated the ability of the phage to infect the plated bacterial strain (positive result), and the absence of clear plaques indicated the lack of ability of the phage to infect the plated bacterial strain (negative result). The results are summarized in Tables 1 and 2. The testing showed the following results.

In comparison to its parent phage CBA120, CBA120-SPTD1.chiTSP2 (RBP-CBA120-2) gained the ability to infect Citrobacter sedlakii, but lost the ability to infect E. coli O157:H7. TSP1, TSP3 and TSP 4 of RBP-CBA120-2 maintained the phage's ability infect to Salmonella Minnesota, E. coli 077 and E. coli 078 respectively.

In comparison to its parent phage CBA12, CBA120-SPTD1.chiTSP3 (RBP-CBA12-1) gained the ability to infect the same Salmonella strains that SPTD1 is able to infect, but lost the ability to infect E. coli 077. TSP1, TSP2, and TSP4 of CBA120-SPTD1.chiTSP3 (RBP-CBA120-1) maintained the phage's ability to infect Salmonella Minnesota, E. coli O157:H7 and E. coli 078 respectively.

In comparison to its parent phage SPTD1, SPTD1-CBA12.chiTSP4 (RBP-SPTD1-1) gained the ability to infect E. coli 078. TSP 2 of SPTD1-CBA120.chiTSP4 (RBP-SPTD1-1) maintained this phage's ability to infect Citrobacter sedlakii. TSP3 of SPTD1-CBA12.chiTSP4 (RBP-SPTD1-1) maintained this phage's ability to infect the same Salmonella strains that SPTD1 is able to infect.

TABLE 1
Summary of testing of synthetic phages with recombinant TSPs (“Neg” = negative result; “Pos” = positive result)

| | | | phage | | | | |
|---|---|---|---|---|---|---|---|
| | | | CBA120- | CBA120- | SPTD1- | | |
| | | O | SPTD1.chiTSP2 | SPTD1.chiTSP3 | CBA120.chiTSP4 | | |
| Strain | Strain # | Antigen | (RBP-CBA120-2) | (RBP-CBA120-1) | (RBP-SPTD1-1) | SPTD1 | CBA120 |
| Citrobacter | ATCC | | Pos | Neg | Pos | Pos | Neg |
| sedlakii | 51493 | | | | | | |
| E. coli | ATCC | O157:H7 | Neg | Pos | Neg | Neg | Pos |
| | 43888 | | | | | | |
| E. coli | ATCC | O77 | Pos | Neg | Neg | Neg | Pos |
| | 23537 | | | | | | |
| E. coli | ECOR70 | O78 | Pos | Pos | Pos | Neg | Pos |
| Salmonella | SARB 13 | 1, 9, | Neg | Pos (faint) | Pos | Pos | Neg |
| enterica, | | 12[Vi] | | | | | |
| subsp. enterica, | | | | | | | |
| serovar Dublin | | | | | | | |
| Salmonella | ATCC | 9, 12[Vi] | Neg | Pos | Pos | Pos | Neg |
| enterica, | 6539 | | | | | | |
| subsp. enterica, | | | | | | | |
| serovar Typhi | | | | | | | |
| Salmonella | AUG053 | 4, [5], 12 | Neg | Pos | Pos | Pos | Neg |
| enterica, | | | | | | | |
| subsp. enterica, | | | | | | | |
| serovar | | | | | | | |
| Brandenburg | | | | | | | |
| Salmonella | 103 | 4, 12 | Neg | Pos | Pos | Pos | Neg |
| enterica, | | | | | | | |
| subsp. enterica, | | | | | | | |
| serovar | | | | | | | |
| Monophasic | | | | | | | |
| Salmonella | ATCC | 6, 7, [Vi] | Neg | Pos | Pos | Pos | Neg |
| enterica, | BAA-1714 | | | | | | |
| subsp. enterica, | | | | | | | |
| serovar | | | | | | | |
| Paratyphi C | | | | | | | |
| Salmonella | MH57137 | 6, 8, 20 | Neg | Pos | Pos | Pos | Neg |
| enterica, | | | | | | | |
| subsp. enterica, | | | | | | | |
| serovar | | | | | | | |
| Newport | | | | | | | |
| Salmonella | DMSO12 | 35 | Neg | Pos | Pos | Pos | Neg |
| enterica, | | | | | | | |
| subsp. enterica, | | | | | | | |
| serovar | | | | | | | |
| Alachua | | | | | | | |
| Salmonella | ATCC | 6, 7 | Neg | Pos | Pos | Pos | Neg |
| enterica, | 7001 | | | | | | |
| subsp. enterica, | | | | | | | |
| serovar | | | | | | | |
| Choleraesuis A | | | | | | | |
| Salmonella | SL1302 | 1, 9, 12 | Neg | Pos | Pos | Pos | Neg |
| enterica, | | | | | | | |
| subsp. enterica, | | | | | | | |
| serovar | | | | | | | |
| Enteritidis | | | | | | | |
| Salmonella | ATCC | 1, 4, [5], | Neg | Pos | Pos | Pos | Neg |
| enterica, | 14028 | 12 | | | | | |
| subsp. enterica, | | | | | | | |
| serovar | | | | | | | |
| Typhimurium | | | | | | | |
| Salmonella | ATCC | 1, 2, 12 | Neg | Pos | Pos | Pos | Neg |
| enterica, | 9150 | | | | | | |
| subsp. enterica, | | | | | | | |
| serovar | | | | | | | |
| Paratyphi A | | | | | | | |

TABLE 2
Results of testing of select synthetic phages with recombinant TSPs, cross-referenced to working examples.

| | Parent Phage; | | | | | | | | |
|---|---|---|---|---|---|---|---|---|---|
| Synthetic Phage; | TSP replaced; | Parent Phage Phenotype | | | | Synthetic Phage Phenotype | | | |
| Working Example | recombinant TSP | TSP1 | TSP2 | TSP3 | TSP4 | TSP1 | TSP2 | TSP3 | TSP4 |
| CBA120- | CBA120; | Salmonella | E. coli | E. coli | E. | Salmonella | E. coli | Salmonella | E. coli |
| SPTD1.chiTSP3 | TSP3; | O21 | O157:H7 | O77 | coli | O21 | O157:H7 | O2, 4, 9, 35 | O78 |
| (RBP-CBA120-1); | CBA120-SPTD1 | | | | O78 | | | | |
| Example 1 | TSP3 | | | | | | | | |
| CBA120- | CBA120: | Salmonella | E. coli | E. coli | E. | Salmonella | Citrobacter | E. coli | E. coli |
| SPTD1.chiTSP2 | TSP2 | O21 | O157:H7 | O77 | coli | O21 | sedlakii | O77 | O78 |
| (RBP-CBA120-2); | CBA120-SPTD1 | | | | O78 | | | | |
| Example 2 | TSP2 | | | | | | | | |
| SPTD1- | SPTD1; | NA | Citrobacter | Salmonella | NA | NA | Citrobacter | Salmonella | E. coli |
| CBA120.chiTSP4 | TSP4 | | sedlakii | O2, 4, 9, 35 | | | sedlakii | O2, 4, 9, 35 | O78 |
| (RBP-SPTD1-1) | SPTD1-CBA120 | | | | | | | | |
| Example 3 | TSP4 | | | | | | | | |
| SPTD1- | SPTD1 | NA | Citrobacter | Salmonella | NA | Salmonella | Citrobacter | Salmonella | NA |
| CBA120.chiTSP1 | TSP1 | | sedlakii | O2, 4, 9, 35 | | O21 | sedlakii | O2, 4, 9, 35 | |
| (RBP-SPTD1-2) | SPTD1-CBA120 | | | | | | | | |
| Example 4 | TSP1 | | | | | | | | |
| SPTD1- | SPTD1- | Salmonella | Citrobacter | Salmonella | NA | Salmonella | Salmonella, | Salmonella | NA |
| CBA120.chiTSP1- | CBA120.chiTSP | O21 | sedlakii | O2, 4, 9, 35 | | O21 | O3, O4 | O2, 4, 9, 35 | |
| Det7.chiTSP2 | (RBP-SPTD1-2) | | | | | | | | |
| (RBP-SPTD1-3) | TSP2 | | | | | | | | |
| Example 5 | SPTD1-Det7 TSP2 | | | | | | | | |
| SPTD1- | SPTD1- | Salmonella, | Salmonella, | Salmonella | NA | Salmonella | Salmonella, | Salmonella | Salmonella |
| CBA120.chiTSP1- | CBA120.chiTSP1- | O21 | O3 | O2, 4, 9, 35 | | O21 | O3 | O2, 4, 9, 35 | O8 |
| Det7.chiTSP2 | Det7.chiTSP2 | | | | | | | | |
| TR2.chiTSP4 | (RBP-SPTD1-3) | | | | | | | | |
| (RBP-SPTD1-5) | | | | | | | | | |
| Example 9 | | | | | | | | | |

### Example 7: Construction of Synthetic Phage Based on SPTD1 Parent Phage

Synthetic phage based on CBA120 parent phage is constructed by replacing TSP1, TSP3, and TSP4 of CBA120 with recombinant TSPs specific for E. coli serotype 0157, which is of great concern for food safety due to the serotype's ability to cause severe disease following human infection. Synthetic phage based on CBA120 and specific for E. coli serotype 0157 is used to detect E. coli serotype 0157 in food samples with improved specificity and reduced false positive detection rate.

### Example 8: Construction of Synthetic Phage Based on SPTD1 Parent Phage

SPTD1 phage is capable of recognizing both Salmonella and Citrobacter. Synthetic phage is constructed by replacing one or more TSPs recognizing Citrobacter with one or more TSPs not capable of recognizing Citrobacter. The resulting synthetic phage is used for Salmonella detection with reduced false positive detection rate in samples containing background bacterial flora including Citrobacter, thus improving the specificity of Salmonella detection.

The resulting synthetic phage is also used to improve phage therapy in chickens to reduce Salmonella colonization and improve food safety. Citrobacter is often a member of healthy chicken microbiome and would be targeted by SPTD1 phage. Thus, disruption of the native microbiome (dysbiosis) in chickens may result from phage therapy with SPTD1. Dysbiosis is associated with detrimental outcomes, including increased risk of infections. Synthetic phage, based on SPTD1 but not capable of recognizing Citrobacter, is used for phage therapy in chickens, which the killing of Citrobacter present in commensal gut flora, thus improving phage therapy outcomes.

### Example 9: Construction of Synthetic Phage for Salmonella Recognition

SPTD1 phage is able to infect Salmonella Typhimurium, but not Salmonella Anatum or Minnesota strains, while also infecting Citrobacter. Replacing C-terminal region of SPTD1 TSP1 with C-terminal region of CBA120 TSP1 C-terminal region confers on SPTD1 the ability to recognize Salmonella Minnesota, and replacing SPTD1 TSP2 C-terminal region with Salmonella phage Det7 TSP2 C-terminal region confers on SPTD1 the ability to recognize Salmonella Anatum, while removing the ability to recognize Citrobacter. Additionally, replacing SPTD1 TSP4 with TR2 Podovirus TSP gave SPTD1 the ability to infect Salmonella Kentucky resulting in a Salmonella-specific phage capable of infecting a wider range of Salmonella species than parent phage SPTD1.

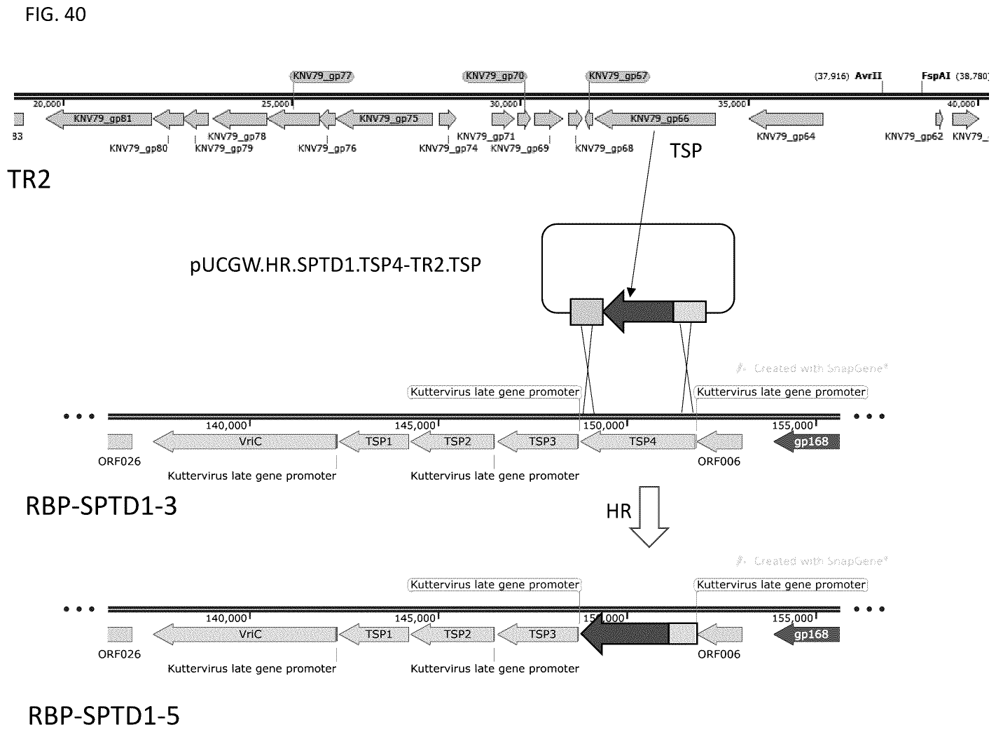

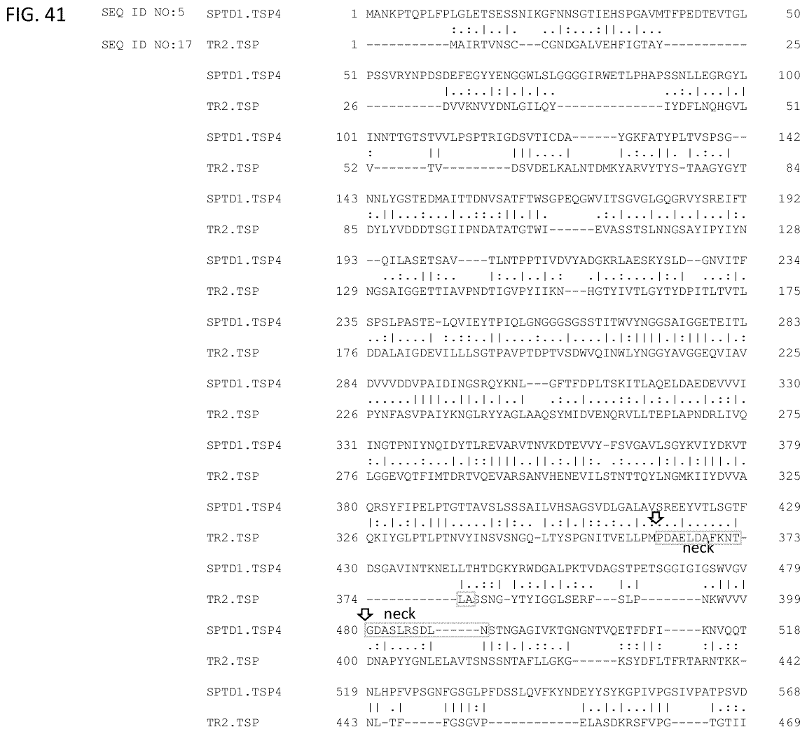

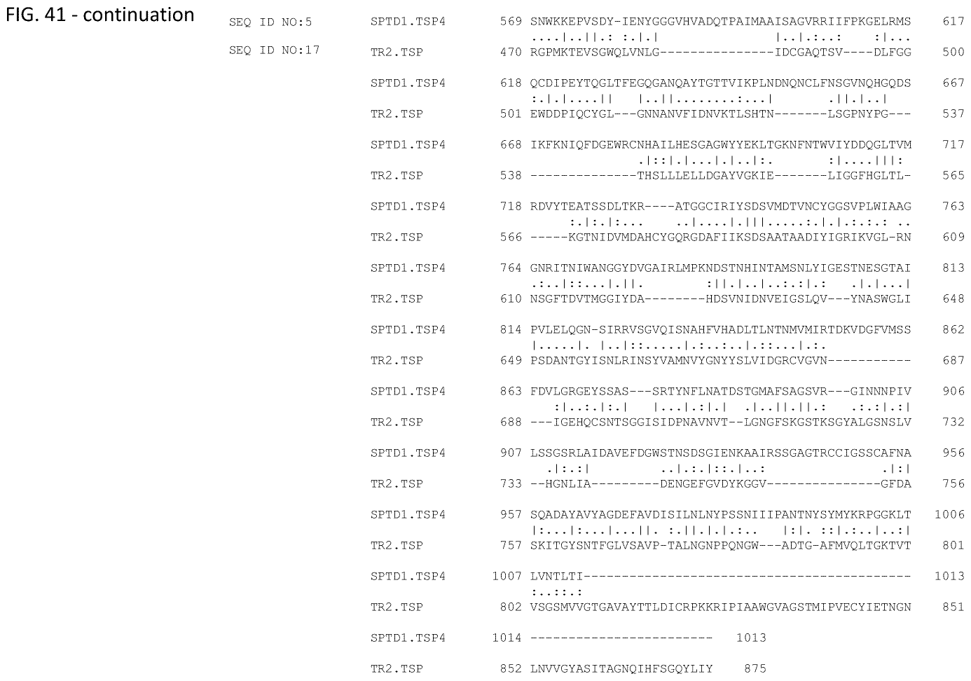

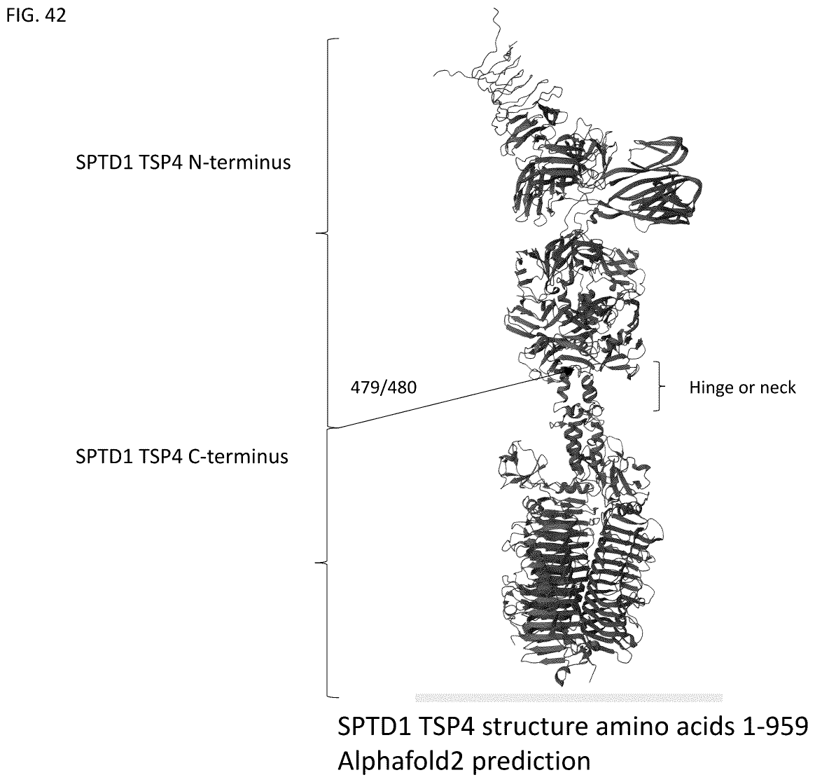

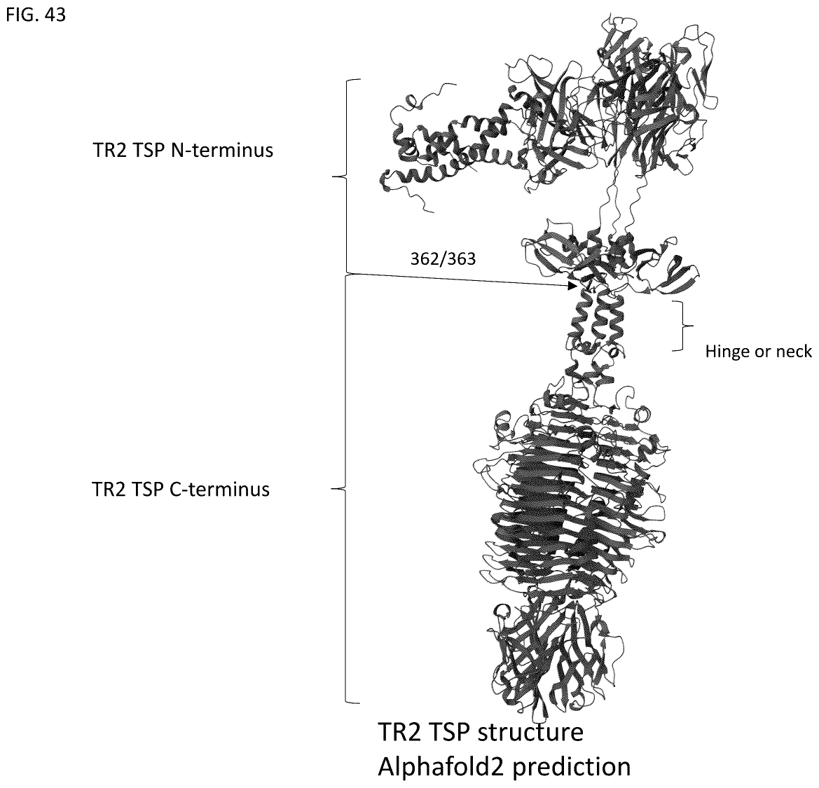

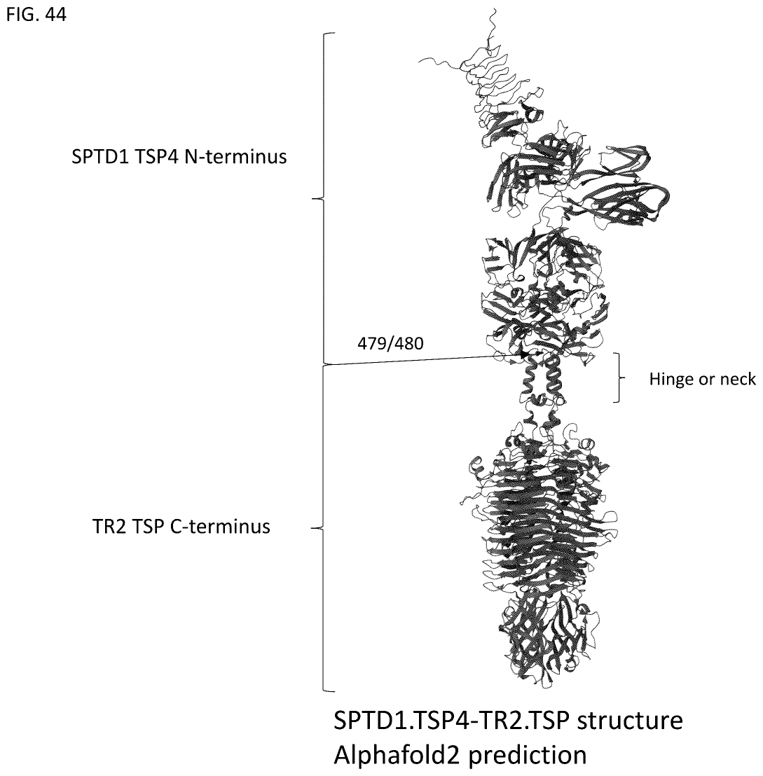

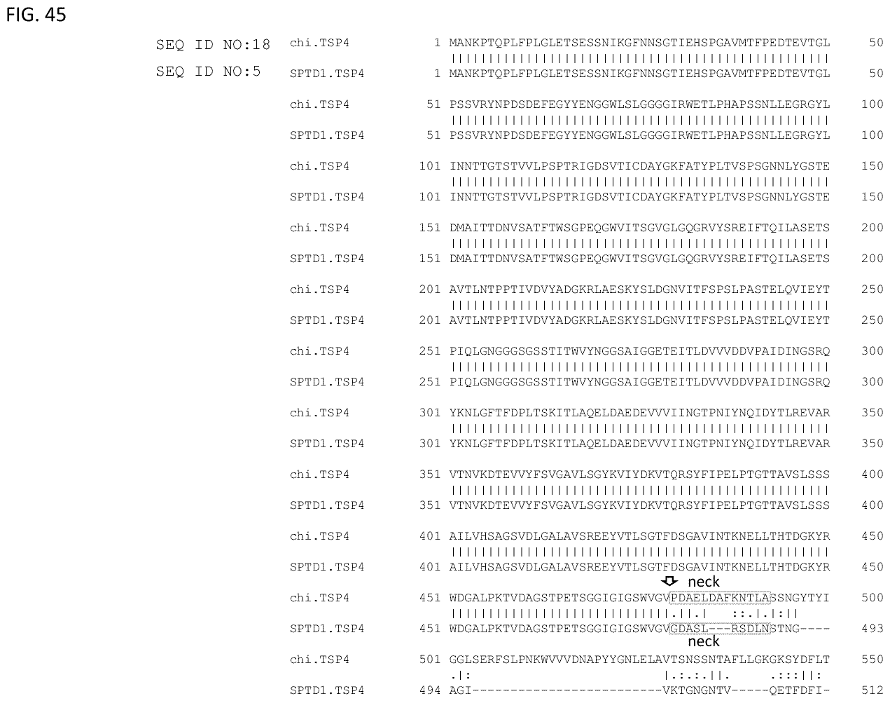

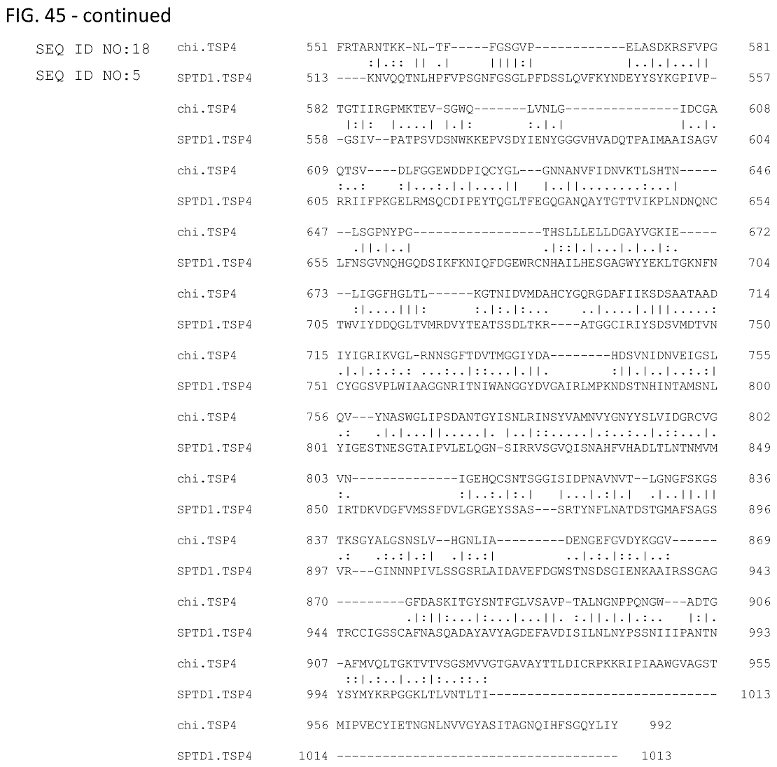

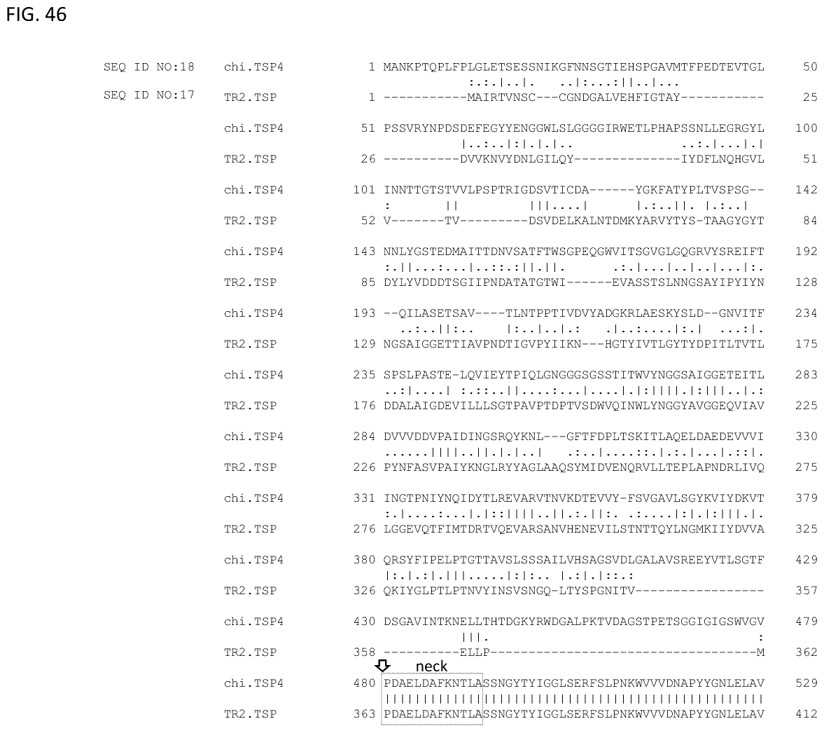

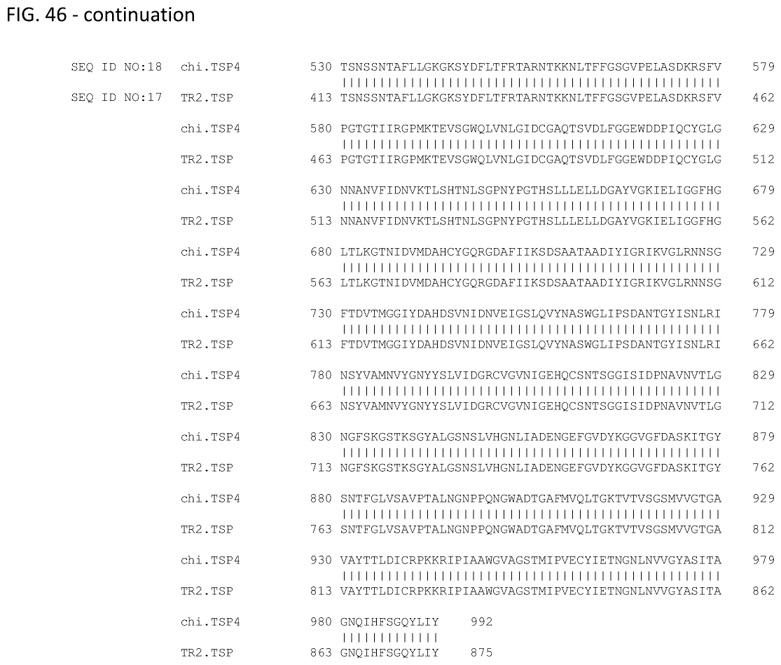

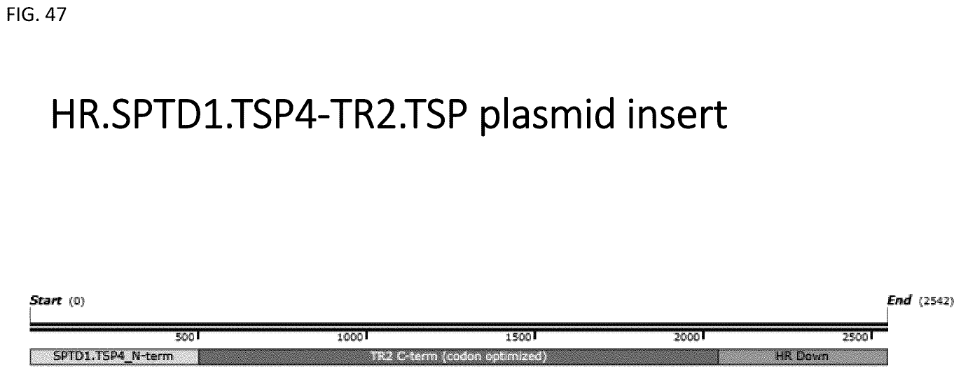

An exemplary synthetic phage was constructed from synthetic parent phage RBP-SPTD1-3, discussed in Example 5, by replacing its TSP4 with a recombinant TSP 4 (“SPTD1.TSP4-TR2.TSP”) containing N-terminal domain from SPTD1 TSP4 and C-terminal domain of KVN79_gp66 (“TR2.TSP”) from podovirus Salmonella phage vB_SalP_TR2 (“TR2”), GenBank Accession No. NC_055921. Nucleic acid sequence encoding SPTD1.TSP4-TR2.TSP was produced, and synthetic phage termed RBP-SPTD1-5 was constructed using the procedures substantially similar to those described in the previous examples. Due to the substantial evolutionary distance between SPTD1 (a myovirus) and TR2 (a podovirus), the C-terminal region of TR2 TSP was codon-optimized to match the late gene codon usage of SPTD1. FIG. 40 is a schematic illustration of the homologous recombination process used to generate synthetic phage RBP-SPTD1-5 from RBP-SPTD1-3 by exchanging TSP4 of RBP-SPTD1-3 with a recombinant TSP4 (“SPTD1.TSP4-TR2.TSP”) containing N-terminal domain from SPTD1 TSP4 and C-terminal domain from TR2 TSP. FIG. 41 illustrates pairwise alignment, performed on Emboss Needle, of amino acid sequences of SPTD1 TSP4 and TR2 TSP. The arrow indicates the joining site between amino acid 479 of SPTD1 TSP2 and amino acid 363 of TR2 TSP. The “neck” regions based on published TSP structure homology and AlphaFold2 predictions are indicated by boxes. FIG. 42 is a schematic illustration of the structure of the SPTD1 TSP4 protein generated by AlphaFold2 including amino acids 1 through 959. FIG. 43 is a schematic illustration of the structure of the TR2 TSP protein generated by AlphaFold2. FIG. 44 is a schematic illustration of the predicted structure of a recombinant TSP containing N-terminus of SPTD1 TSP4 and C-terminus of TR2 TSP. FIG. 45 illustrates pairwise alignment, performed on Emboss Needle, of amino acid sequences of chimeric SPTD1.TSP4-TR2.TSP (“chi.TSP4”) and SPTD1 TSP4 (“SPTD1.TSP4”). The arrow indicates the joining site between amino acid 479 of SPTD1 TSP4 and amino acid 363 of TR2 TSP. FIG. 46 illustrates pairwise alignment, performed on Emboss Needle, of amino acid sequences of recombinant SPTD1.TSP4-TR2.TSP (“chi.TSP4”) and TR2 TSP (“TR2.TSP”). The arrow indicates the joining site between amino acid 479 of SPTD1 TSP4 and amino acid 363 of TR2 TSP. FIG. 47 is a schematic illustration of HR.SPTD1.TSP4-TR2.TSP plasmid insert. Upstream homologous recombination region is 500 bp of the SPTD1 TSP4 N-terminal region.

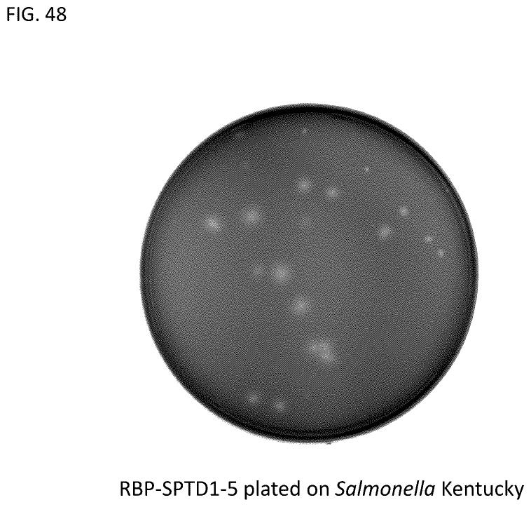

Plating experiments were performed to test the ability of synthetic phage RBP-SPTD1-5 to infect Salmonella Kentucky and other target strains. FIG. 48 is a photographic image of the plate culture illustrating the results of the plating of synthetic bacteriophage RBP-SPTD1-5 on Salmonella Kentucky. Limit of Detection Assays (LOD) were performed with RBP-SPTD1-5 to demonstrate the ability of the synthetic phage with chimeric TSP1, TSP2, and TSP4 to infect target Salmonella strains, including Salmonella Minnesota (TSP1), Salmonella anatum (TSP2), Salmonella enterica Typhimurium 19585 (TSP3), and Salmonella Kentucky (TSP4). These results are summarized in Tables 3A-3E.

Table 3. Limit of Detection (LOD) of the modified TSP 1, 2 and 4 of RBP-SPTD1-5NL and unmodified TSP3.

A.
RBP-SPTD1-5.NL S. Minnesota 52329.1 [TSP1]

| Calculated | CFU in | | | | | |
|---|---|---|---|---|---|---|
| # Cells | 100 ul | Avg. | SD | RSD | Signal-BG | S/B |
| 0 | — | 101 | 15.1 | 14.9 | 0 | 1.0 |
| 1 | 0.5 | 227 | 258.3 | 113.7 | 126 | 2.2 |
| 2 | 2.5 | 214 | 139.4 | 65.0 | 113 | 2.1 |
| 5 | 5.8 | 388 | 488.3 | 125.9 | 287 | 3.8 |
| 10 | 8.2 | 632 | 451.3 | 71.4 | 531 | 6.2 |
| 100 | 95.5 | 5595 | 2163.1 | 38.7 | 5494 | 55.3 |
| 1000 | 1005 | 55219 | 6244.1 | 11.3 | 55118 | 545.4 |
| 10000 | — | 736715 | 122898.7 | 16.7 | 736613 | 7276.2 |

B.
RBP-SPTD1-5.NL S. enterica Typhimurium 19585 [TSP3]

| Calculated | CFU in | | | | | |
|---|---|---|---|---|---|---|
| # Cells | 100 ul | Avg. | SD | RSD | Signal-BG | S/B |
| 0 | — | 131 | 14.2 | 10.9 | 0 | 1.0 |
| 1 | 0.8 | 205 | 156.0 | 76.0 | 74 | 1.6 |
| 2 | 1.5 | 710 | 727.6 | 102.5 | 579 | 5.4 |
| 5 | 1.5 | 2147 | 1307.3 | 60.9 | 2016 | 16.4 |
| 10 | 5.8 | 1680 | 1011.8 | 60.2 | 1549 | 12.8 |
| 100 | 52.5 | 32067 | 5662.6 | 17.7 | 31936 | 244.5 |
| 1000 | 542.5 | 448788 | 164454.9 | 36.6 | 448657 | 3421.5 |
| 10000 | — | 11047179 | 1157849.4 | 10.5 | 11047048 | 84222.5 |

C.
RBP-SPTD1-5.NL S. Anatum SLR377 [TSP2]

| Calculated | CFU in | | | | | |
|---|---|---|---|---|---|---|
| # Cells | 100 ul | Avg. | SD | RSD | Signal-BG | S/B |
| 0 | — | 111 | 13.8 | 12.4 | 0 | 1.0 |
| 1 | 1 | 192 | 161.9 | 84.5 | 81 | 1.7 |
| 2 | 1.5 | 294 | 413.0 | 140.6 | 183 | 2.7 |
| 5 | 4.8 | 1051 | 1020.4 | 97.1 | 941 | 9.5 |
| 10 | 9 | 2370 | 1841.4 | 77.7 | 2259 | 21.4 |
| 100 | 98 | 21267 | 7159.9 | 33.7 | 21157 | 192.3 |
| 1000 | 830 | 203481 | 39100.7 | 19.2 | 203371 | 1840.1 |
| 10000 | — | 4029516 | 784465.0 | 19.5 | 4029405 | 36438.7 |

D.
RBP-SPTD1-5.NL S. Kentucky 1315 [TSP4]

| Calculated | CFU in | | | | | |
|---|---|---|---|---|---|---|
| # Cells | 100 ul | Avg. | SD | RSD | Signal-BG | S/B |
| 0 | — | 117 | 18.9 | 16.1 | 0 | 1.0 |
| 1 | 0.5 | 179 | 181.3 | 101.2 | 62 | 1.5 |
| 2 | 1.8 | 234 | 265.5 | 113.7 | 116 | 2.0 |
| 5 | 3.5 | 184 | 217.0 | 118.1 | 66 | 1.6 |
| 10 | 10.5 | 1051 | 808.0 | 76.9 | 933 | 8.9 |
| 100 | 76 | 5790 | 2022.3 | 34.9 | 5672 | 49.3 |
| 1000 | 705 | 44150 | 10515.8 | 23.8 | 44032 | 376.0 |
| 10000 | — | 596699 | 152013.5 | 25.5 | 596582 | 5081.9 |

E.
SPTD1.NL Reference

| Salmonella strain | SPTD1.NL (RLU) |
|---|---|
| S. Typhimurium 19585 | 365391456 |
| S. Minnesota 52329.1 | 45 |
| S. Kentucky 1315 | 47 |
| S. Anatum SLR377 | 192 |

### Example 10: Construction of a TSP-Deficient Synthetic Phage for Rapid Modularity

A member of the Ackermannviridae family, such as CBA120, is used to create a synthetic TSP-deficient (ATSP) phage to use as a modular platform capable for rapid generation of synthetic phage with customizable TSP complexes. One or more TSPs are provided in trans, for example, encoded on a bacterial plasmid, to complement a TSP deficiency. Generation of a conditionally replicative TSP-deficient phage is illustrated in FIG. 3 and involves the use of HR to replace the TSP gene cluster with a tractable marker or reporter, such as a luciferase. The synthetic TSP-deficient phage is expected to obtain TSPs from those encoded by wild-type copies of the phage genome present in the same cell at the time of recombination. After this step, the recombinants must be isolated and maintained on a bacterial strain transformed with a plasmid encoding TSPs or another mechanism of in trans delivery of TSPs. TSP-deficient phage is conditionally replicative and will only produce infectious progeny in the appropriate complementing strains. This dependence on in trans TSPs is exploited as follows.

For example, when CBA120.ATSP phage is prepared in a bacterial strain expressing TSP1, TSP2, TSP3, and TSP4 from CBA120, the resulting progeny phage recognizes the same strains as the CBA120 parent. In another example, when CBA120.ATSP is prepared in a bacterial strain expressing TSP1, TSP2, TSP3, and TSP4 from SPTD1, the progeny phage recognizes the same strains as the SPTD1 donor. This is illustrated om FIG. 4, where a synthetic phage that does not encode for its own TSPs adopts the TSPs (and thus host range) of the donor TSPs provided in trans. In some cases, in trans TSPs may also be chimeric TSPs, with N-terminal regions equivalent to CBA120 TSPs and C-terminal regions equivalent to any other known TSP. Thus, a modular platform is created from a TSP-deficient Ackermannviridae phage allowing rapid generation of progeny phage of customized host ranges (capacity).

### Example 11: Phage Sequences

TABLE 4
Phage sequences.

| Sequence name | SEQ ID | Type of sequence |
|---|---|---|
| SPTD1 phage | SEQ ID: 1 | DNA (genomic sequence) |
| SPTD1 Tail-spike protein 1 (TSP1) | SEQ ID: 2 | amino acid sequence |
| SPTD1 Tail-spike protein 2 (SPTD TSP2) | SEQ ID: 3 | amino acid sequence |
| SPTD1 Tail-spike protein 3 (SPTD TSP3) | SEQ ID: 4 | amino acid sequence |
| SPTD1 Tail-spike protein 4 (SPTD1 TSP4) | SEQ ID: 5 | amino acid sequence |
| SPTD1-CBA120.chiTSP1-Det7.chiTSP2 | SEQ ID: 6 | DNA (genomic sequence) |
| (RBP-SPTD1-3) phage | | |
| RBP-SPTD1-3 TSP1 Tail-spike protein 1 | SEQ ID: 7 | amino acid sequence |
| (RBP-SPTD1-3 TSP1; SPTD1- | | |
| CBA120.TSP1) | | |
| RBP-SPTD1-3 TSP2 Tail-spike protein 1 | SEQ ID: 8 | amino acid sequence |
| (RBP-SPTD1-3 TSP2; SPTD1-Det7.TSP2; | | |
| SPTD1.Chi.TSP2) | | |
| CBA120 Tail-spike Protein 1 | SEQ ID: 15 | amino acid sequence |
| CBA120 Tail-spike Protein 2 | SEQ ID: 9 | amino acid sequence |
| (CBA120_TSP2) | | |
| CBA120 SPTD1 chimeric Tail-spike | SEQ ID: 10 | amino acid sequence |
| Protein 2 (CBA120-SPTD1_TSP2) | | |
| CBA120 Tail-spike Protein 3 | SEQ ID: 11 | amino acid sequence |
| (CBA120_TSP3) | | |
| CBA120 SPTD1 chimeric Tail-spike | SEQ ID: 12 | amino acid sequence |
| Protein 3 (CBA120-SPTD1_TSP3) | | |
| CBA120 Tail-spike Protein 4 | SEQ ID: 13 | amino acid sequence |
| SPTD1 CBA120 chimeric Tail-spike | SEQ ID: 14 | amino acid sequence |
| Protein 4 (SPTD1-CBA120_TSP4) | | |
| Det7 Tail-spike Protein 2 (Det7 TSP2) | SEQ ID: 16 | amino acid sequence |
| TR2 Tail-spike Protein (TR2_TSP) | SEQ ID: 17 | amino acid sequence |
| SPTD1 TSP4 TR2 chimeric Tail-spike | SEQ ID: 18 | amino acid sequence |
| Protein (SPTD1.TSP4-TR2.TSP; chiTSP4) | | |

### Sequence Listing

The instant application contains a Sequence Listing which has been submitted electronically in xml format and is hereby incorporated by reference in its entirety. The sequence listing does not go beyond the disclosure in the present application as filed. The file with the sequence listing, created on Nov. 9, 2023, is named 057618-1414122 PhDx 2022-01-US.xml and is 348,160 bytes in size.

It is understood that the examples and embodiments described in the present disclosure are for illustrative purposes only and that various modifications or changes in light thereof will be suggested to persons skilled in the art and are to be included within the spirit and purview of this application and scope of the appended claims. All publications, patents, and patent applications cited in the present disclosure are hereby incorporated by reference in their entirety for all purposes.

### PUBLICATIONS CITED IN THE DISCLOSURE

Nobrega et al. Targeting mechanisms of tailed bacteriophages. Nat Rev Microbiol 16, 760-773 (2018). doi: 10.1038/s41579-018-0070-8[0187]
Prokhorov et al. Function of bacteriophage G7C esterase tailspike in host cell adsorption. Mol Microbiol. 105(3):385-398 (2017). doi: 10.1111/mmi.13710
Leiman et al. Morphogenesis of the T4 tail and tail fibers. Virol J 7, 355 (2010). doi: 10.1186/1743-422×-7-355
Sørensen et al. Subtypes of tail spike proteins predicts the host range of Ackermannviridae phages. Comput Struct Biotechnol J. August 21; 19:4854-4867 (2021). doi: 10.1016/j.csbj.2021.08.030
Greenfield et al. Structure and function of bacteriophage CBA120 ORF211 (TSP2), the determinant of phage specificity towards E. coli O157:H7. Sci Rep. 10(1):15402. (2020) doi: 10.1038/s41598-020-72373-0
Plattner et al. Structure and Function of the Branched Receptor-Binding Complex of Bacteriophage CBA120. J Mol Biol. 431(19):3718-3739 (2019). doi: 10.1016/j.jmb.2019.07.022
Walter et al. Structure of the receptor-binding protein of bacteriophage det7: a podoviral tail spike in a myovirus. J Virol. 82(5):2265-73 (2008). doi: 10.1128/JVI.01641-07
Bertozzi Silva et al. Host receptors for bacteriophage adsorption. FEMS Microbiol Lett. 363(4):fnw002. (2016) doi: 10.1093/femsle/fnw00 Ackermann Bacteriophage taxonomy. Microbiology Australia 32(2):90-94 (2011)
Henikoff and Henikoff, 1989, “Amino acid substitution matrices from protein blocks” Proc. Natl. Acad. Sci. USA 89:10915-10919.
Karlin and Altschul, 1993, “Applications and statistics for multiple high-scoring segments in molecular sequences.” Proc. Nat′l. Acad. Sci. USA 90:5873-5787.
Chao et al. Structure of Escherichia coli O157:H7 bacteriophage CBA120 tailspike protein 4 baseplate anchor and tailspike assembly domains (TSP4-N). Sci Rep. 12(1):2061 (2022) doi: 10.1038/s41598-022-06073-2. PMID: 35136138; PMCID: PMC8825819

What is claimed is:

1. A synthetic phage comprising at least one recombinant tail-spike protein (TSP) comprising an N-terminal region and a C-terminal region, wherein (a) a combination of the N-terminal region and at least a part of the C-terminal region is engineered in a laboratory, and/or (b) the C-terminal region comprises one or more engineered amino acid sequences, wherein the synthetic phage is constructed from a parent phage that is an Ackermannviridae phage.

2. The synthetic phage of claim 1, wherein the C-terminal region is capable of recognizing a target host.

3. The synthetic phage of claim 1, wherein the at least one recombinant TSP confers on the synthetic phage an ability to recognize the target host, wherein the ability to recognize the target host was absent in the parent phage.

4. The synthetic phage of claim 3, wherein the C-terminal region of the at least one recombinant TSP comprises at least one amino acid sequence occurring in a C-terminal region of a TSP from a phage different from the parent phage and capable of recognizing the target host.

5. The synthetic phage of claim 4, wherein the phage different from the parent phage is a non-Ackermannviridae phage

6. The synthetic phage of claim 1, wherein the at least one recombinant TSP has at least 70%, at least 75%, at least 80%, at least 85%, at least 90%, at least 95%, or at least 99% sequence similarity to SEQ ID s 6, 7, 8, 10, 12, 14, or 18.

7. A recombinant tail-spike protein (TSP) comprising an N-terminal region comprising amino acid sequences derived from an Ackermannviridae phage and a C-terminal region, wherein: (a) a combination of the N-terminal region and at least a part of the C-terminal region is engineered in a laboratory, and/or (b) the C-terminal region comprises one or more engineered amino acid sequences.

8. The recombinant TSP of claim 7, wherein the C-terminal region is capable of recognizing a target host.

9. The recombinant TSP of claim 7, wherein amino acid sequences of the C-terminal region and the N-terminal region are derived from different phages, and/or wherein the amino acid sequences of the C-terminal region are derived from a non-Ackermannviridae phage.

10. A nucleic acid sequence encoding the recombinant TSP of claim 7.

11. A method of altering a phage host range, comprising altering a parent phage that is an Ackermannviridae phage to include at least one recombinant tail-spike protein (TSP) wherein the at least one recombinant TSP is the recombinant TSP of any one of claim 7, thereby generating a synthetic phage with an altered host range.

12. The method of claim 11, wherein the altered host range is broader or narrower than a host range of the parent phage.

13. The method of claim 11, wherein the altering the parent phage comprises altering a C-terminal region of at least one tail-spike protein (TSP) of the parent phage.

14. The method of claim 13, wherein the altering the C-terminal region of at least TSP of the parent phage comprises exchanging the at least a part of the C-terminal region of the TSP of the parent phage for the C-terminal region of the recombinant TSP.

15. A method of detecting the target host using the synthetic phage of claim 2, comprising:
contacting a sample with the synthetic phage for a time sufficient for the synthetic phage to infect the target host; and,
detecting the synthetic phage or progeny phage of the synthetic phage, wherein positive detection of the synthetic phage or the progeny phage of the synthetic phage indicates that the target host is present in the sample.

16. The method of claim 15, wherein the synthetic phage comprises an indicator gene, and the detecting comprises detecting an indicator protein product produced by the synthetic phage or the progeny phage of the synthetic phage, wherein positive detection of the indicator protein product indicates that the target host is present in the sample.

17. A kit or a system for performing the method of claim 15, wherein the kit or the system comprises the synthetic phage.

18. A method of controlling a microorganism using the synthetic phage of claim 2, comprising administering to a subject or contacting a sample, an object, an apparatus, or a material with the synthetic phage, wherein the synthetic phage is lytic.

19. A kit or a system for performing the method of claim 18, comprising the synthetic phage.

20. A method of constructing a synthetic phage with desired host-recognition capacity, comprising:
(a) providing virions of a recombinant phage constructed from an Ackermannviridae phage, wherein the virions lack tail-spike protein-encoding genes (TSP-encoding genes) and comprise tail-spike proteins (TSPs) capable of recognizing a bacterial host;
(b) providing the bacterial host comprising one or more plasmids encoding TSPs having the desired host-recognition capacity;
(c) infecting the bacterial host with the virions of the recombinant phage; and,
(d) allowing progeny phage to be produced in the bacterial host, wherein virions of the progeny phage lack TSP-encoding genes and comprise TSPs encoded by the one or more plasmids in the bacterial hosts, wherein the progeny phage is the synthetic phage with the desired host-recognition capacity.


SEQUENCE LISTING (18 sequences)

SEQ ID NO: 1 - DNA, 157533 bp, Kuttervirus SPTD1
  source 1..157533: mol_type = genomic DNA
atggcgtatcagaagcgtgatatagaatatgccccaattaaaacggggttcaaaatagacgatgtgaaattgcgtatggaccaaacgttcatgcgcaaaccttctgtgcgtgctcctcgagtcgagttaatgctgacagatgaacaagaagacgaattcgttgaatgttctatggatgcacactacttcgccgccaactattacaagataaccacgatcgataagggctttatccttttcgatatgcatgattatcagaagcagttgttccacgactttcaggactatcgatttaatgcggtcgtccaggctcgtcagtccggaaaatgtgtaaaaggcgacacgcttgtcacagtttacgatactctcagccaagaggagttgctcctgactataggggagcttcacagtcgcttcgaggacgtcaaccacgctgtgccgctgaataccattggcaaccatgacaagttcgtggacagccgcttcggcaaacgttactttgtacaatccgatagtggctgggttccggttatcgcagcgcataagacgaagaaatatgcggaattcgttattgtcaccgttaccggaaggcgaattaacgtggccgatgaacatatgttcttcactccggacatgaaggagatatttgccaaagacctgaatgctgggtcatacatcatgacgacagaaggtccagaagaaatccgtgagatttggcagactggtgaatatcaccatatgtacgatcttcaggtcaaatctagtgatcaacgttattacacgaatggctttctcagtcataacacgaccgtggtggcggcgtttcttctttggtatgcgatgttccattctgataaggaaatcgcagtactggcaaacaaagagaaacaagcgatagaaattcttgaccgtatcagaaaagcataccaagaccttccattcttccttcagcagggttgtgagaagtttggttctaccctgatagagtttgagaatggttctaagatatacgcttatgccacgtcttcggactccatccgcggtcgttctgtatcgctcttgtacgttgacgaagtcgcgtttatcgaaaacgactttgaattttgggaatcaactttcccagccattgcatctgccgatacatcacgctgtattttgacaagtactccgaaaggccagcgagggttgttctatgatattgtcaccaaagcagatccgcgccatccacaatacaacgacttccacctaactgaagtcccatggtataaggttccggcatataccaaagatccagattgggaaaccaaacaacgtgcccgtctgggggatgctcgtttcgatcaagaatttggcattaagttccgtggttccgtgggttcattgattccggccaaatgcttagataaaatgacgtccaagttgtatcgggaacctaatgaattcaccaagatttataaggaatacgacccacaacgtctctactttgggattgcggacactgggaagggggtggaaggagattattccgtcttaacaattctggatataactgaatatccgcatgttatagcagccaagtacaggaataacacgatacctcctatgatgtatgcatacacgatagctgatatgtgcaccgaatacggggaatgccctgtccttgttgaaacaaacaacgacgtcggcggtcaggttattacaattctttatcaagagatagaatatccagaaatcatattcacatcaacagataacaaagggacggggaaacggattggtggacgtaaaccggaacctggtatcaataccaacagaaaagtccgatctatcggttgtgctaacctgaaagcgctcattgagaaagaaatgttggtgatagaagaccaggacacgatagatgaactcagtacatttgtgttcaaaggcgctcggtatgaagccgatgacggttgtcacgatgactgtgtcatgccgttggtcttatattcatgggcggtaaaacaagaatggttcagtgatttgacatctacaagtatttctcaggacatgagaaaccgaatgtcttcaactgaatctcagcaggtattcccatttggtggtttagtcgttggagacaccccatccgaaactgagcatttgcctggtttcgggggtattcaagttttcaacgaaatgtcagggatgaccatggatgagtggtttaagaattgatcactaaatatctttcaaattgagcgaaactctaccgagaaggaatgatattatggcaactcaaagcttcagcgttgcgccgtccgttcagtggactgagcgtgatgccacgcttcagacgtctccgtccgttgttgttcagggcgcgaccgtcggcaaatttcaatggggtgaagttgaacttccagtgctggtgactggtggtgagacaggtttggtgaagaaattcttcaaacccaacgacagtactgcgacagatttccttgtgattgcagactttttgtcttacagctctatggcatgggtgacccgtgttgttggtcctctggccaagaactctgttaccaaaggtcagacagcgattgcgatcaaaaacaaactggattttgaaacagcaagtccttcggcgtccatcacttggactggtcgttatccaggttccctgggtaatgatattgctatcaatgtttgtgatgctgctggattctcaacttgggaattccgtaataactttgcatacgcacctcagtctggtgaattccatgtagttgtcgtagacaaagttggtcgcattaccgactctgctggcgcggtcggtcaggttgaccgtatctccgtttctggtacggctactgctgctggcaccatcagtgtggcaggtgaagatatcgcatatctggatactgatactccagccgatttggcaaccaaaatcggtacagcactaacttccctgacaagtgtttattcttctgttgtcgtgaagtccaacactgtcatcgtgacccataaagctattggtccccagacagttactgctattgttccagatgataaggggctgactgcaaccgccgtgattaccactgttggcgcatctggttctattatcgaaaaatacgaactcatgcagaacacacagggttccaaaaagtccgatggcgcgaatgcgtacttcaaagatgtcatcaatgatacatcaaattgggtgtacaccttcgctaccgctctggccgcaggtgttgttgaattagaaggcggcgtagacgattataacatcaaccgcgtggcagctatccaagtcttgaacaatgctgaagcatatgatgcgaagccagtatttgcgtactgtgaagaactgattgagcaacaagcattgatcgacttatctactgagcgaaaagatacggtatctttcgtatccccgctccgtgatacggttgttggcaaccgtggtcgtgaaatggatgatgtcgttgcttggcgtgaaagccttgttcgcgactcttcttatttcttcatggatgataactgggcatacgtgtacgacaagtacaacgacaaaatgcgttggattccggcttgtggtggcaccgcaggtgtttgggcgcgaagcattgaaatcgcgggtatctacaaatctccggcgttccacaaccgtggtaaatacaacaactataatcgaatggcgtggtctgcatcttctgatgaacgtgctgtgttgtaccgcaaccagatcaacagcatcgtaaccttctccaatgaaggcatcgtcctgtatggtgacaaaactggcctgacccgcccgtcggcgtttgaccgcatcaacgttcgtggtctgttcatcatggctgagcagaacatcgccgcaatcgccaaatactatcttggtgagaacaatgacgcgtttacacgtggtctgttcagcaatgcggttcgtccatatattcgccagctggcaaatatgggcgcgatttacgatggtaaagtcaaatgtgatgaagacaacaacactgctgatatcattgcggcaaaccaaatggttgcaggcatttggttgaagcctgagtacagcataaattgggtgtatcttgactttgctgcagttcgaccagatatggagttcagcgaaatcgaatccggtggcggcatcgttgctgcttcataaaatgaccaaccccgcttcggcggggttttcctttgtcttattgatttgagttattgaatgatatacgcatacaaaattgaaacgataactaatggaaaggtctacatcggtgtgaccaatgacccacaacgaagatatgatcagcattttgcgaatgcttttcatcatggagtccaaagtgaattgtatgatgccctgcgtaagtatggaacatctggatttactttcgttgtaattgctcagaccgatgaagtccataaatgggaattagagaagcaactcatcgctcaatataattcttatgagatgggttacaacatgaataaaggcggtaatgatggaagtcatatgctgggtaaaactgcagcaaaattgacatctacaggtgaacacattggtcttgtttctctcatagatccccgttgggaattgtgtgaaatcgaacccgttttaagcaatgttgaccacggatttgatttttcttccatgaagattaaaagattgacgactaattagaattgtgtactcttatttggagatataaaaatggcgacagtcaatgagtttcgcgcagccatgtcacgagggggcggcgtacaacgccaacaccgctggcgtgtgactgtaaactttccttcttttgttgctggttccgacacaattcgtgacgtgtccttgctggctgtaaccaccaacaccccaacaggtcagctgggcgaaattctggtgccctggggtggtcgtgaacttccgttcccaggggatcgtcgtttcgaagcgcttcctgtgacgttcattaacgtggtgaacaacgctccatacaatgcttttgaagtgtggcagcaattcatcaacggtagtgaaaacaaccgcgccagcgcgaatccagatgattatttccgtgatatcatcatggaacttctggacgcgaatgacaacgtgactaagacatggactttacagggcggctggcctcaaaacctcggccaactggaactcgatatgtcggcaatggactcttatacacagttcactgtcgacttgcgttatttccaagccgtttctgacaagtcattgtaatgtcctcggtgctgggggaatgttattccccctcactaattacaagaaaatgttgaggacttagatcatggctggatacggcagagggttcttcggtttgtttggcggcggcggtttagtcaatgccaaagtcgacaccgataagttagcccaaaaacaagatgaattgttgctcaccaaggcgacagttgttgctctggacgatgctcaagacggttctataatccttcagggcggtgcaaacacctacaactatgttggcgttgagagtgaacttctcagcgttaaaacagttgtggaagaatatcagtccatggcccagcagccagaaatacgcaaagctgtggacatcattgttaatgacgttgtcacatgtgaggaagacgaaactccagtgacagtcaatcttgacaaagttgatggtatttctgaatcaatcaaagaatctatcaccgaatgtttcaaagaagtgattcatttgatggattttgaaaatacggcgtatcaaaagattcggaaatggtatgtagatggtcgacaagcgtatcatgtgattgttgacccaacgaataagaagggtgggatcaagaaattggtcatgctggattctcgttgtattcgtcctgtgtacatcgttgaaaaggcaatgcgtgaaggcggtattgaagcaatagaatctgtaacattgaagtattattacaacccgaattataatcgaaaccaattcactggccaatctggtacttcccagaacttccagccttcacagcaagaactcgtattcgatgacgaaagcattgtttacatcgatagtggtgaagagccattggccaatggtattgtcccaggccttttaaatcctgctatccgtccgttgaacaacctggttacgactgaagatgcgactgtaatttatgccatcactcgcgcccctgagaaacgcgcattctatcttgacgtcggtactctcggcaagaaatctgctgaagaatacatgaccatgatgatgggtaaattcaaaaaccgcaacgcatatgaccgcaccactggtaagataacaggtaatgcccatcttatgggtattgcagaggattattggttgccgcgccgagaaggtcagaatgctacagagattgcgactgttgggggtgggaatcaattgggcgaaatggatcacgtgaactatttccgtgaaaaactctatgatgctctaatgatccctaagagccgcctccaagaggaaggatctattaacattgggggttctaaccttgcggagattacacaggaagagctgcgcttcagcaagttctgtgctgggttacgacgccgttactcccatttctttatggaattcttgcgccgccagttaattttgaaaggcgttacggatgaaaaggattggaatgagaagatcaaaccgtttataaagtttgaattcacatccgatagttacattcgtgaacaacaagaaaacgctatcttgaatgatcgtctggcttcattgaacactgttgagccatttgttggttccattttctccatagactacgtcatgagaaatgtattgcgtatgtctgatgaagaagttaaagaacaacaaacgaagatcgcggaagagaagaagaaaggtctatatccgaaggttcaagcagatgaaactggcaattatagcggttcagatgttagtccgttgaagtttaaacctgagactattccattctccggttcaacagacgatagtatttaaagttcaactaaatatagaaaattaattcggagatcaaaatcatgagcgcaattgatatcgttcgtgcagtaattgacggtgacactgaaactgctgttgcagaatgcaacatggaacttgatgcacgtagtcaagaactattaaaccaaggcacagcatatgttctggattctatcgcagctgatatgaattcaaacaacaacggccagtaaggagatgcatgatggaaatcacagagatcgccacgttcgctgatttcctggcctctcgtatggacgagcagcgagtcatcgataaagtgaacgctcgtggtaaacgccgccgccgcttgaaatgtgcccctgggttcaaattatcggctgacggttcacgttgtgaagtcatggatgccagcgaacgtcgtgtgcgcaaaatcggcaaccgaaaagctcttcgctccaaaaagcgtatgggtatgggctaccaacgaaaaatcgagcgtcgtaagaaaaaggctatgaagttccgcaaaatgatgggactgagtaagaacaagtaaggagtttatgatgaaactgttgcgtgagatcacagcgatagggaaggatcttcaaatcggtgaggatacgacctcaactggtgggaaggccatgttcatcgaaggtccgtttgtgatgtgtaaccaagtaaaccggaacgggcgtaactatgatctacagaaagtgggtatccctgctgtcgaagcatatgacaaagagtatatccaagatcgtcgcgcaatcggtgaagtcacacatcctgactatcctttccctaatctggtggaagcagcgctcaaaaccgaatcccttcgctgggaaggcaccaatgccatcggtcgggcgcgaattttaaatacaccaaaaggccaaatcatacgtgcattggctgaagcagacttcaatctggccgtgtctacacgtggtctgggtgagaccaagtcggtaaacggttatgacgacgttcaacctggctttatgctcaccgctgttgatgcagtcgaccgtccttctggacaagtttgttatgttaaggctgtgagtgaatctgttgaatggcagcttgatgaggcttctggtatttggatgcctcgggatgttaaaggaaacgttgttgatcagttggtgaaagccaatattcaagtagaagacgacttcctgcgccgccttgatgcagcattgaatcatctgggctgaatttcagctcactaaatactgaaaaatcgttcagaaggaaactatcatgaaacctgaattgcaaaaactgtttgaaggcgttaatggcctcagcaccgatttcttggacaaagtatctggtctgctggaatctaaagttgaagccgcccgtctgcaagctattcaagaaactgaagcggctggtaacgttgagcgcctgactctggtagaagcccaccagaaagaagtcgcagaccttaaagaaaatttcactcttcagttggcggaaaaagttgattcgttcctcaacgcggttgttgaagaatgggctaacaaaaatgctccggctattgacgctcagatcaaaaccgaagctgctgaacgcttcctcactggtttctctaatgttctgaaagaagcaggtgttagtttcgccactgacccagacggtcagattgcagctctcaccaaccgcctggctgaagcagagcaacgcgccagcattgccaatactgaattggctcagctcaaagaaagtgaaaccaaacgccagcgcaatgatgtcattgatcgtatttgtgaaggtatggttgacaccaagaaagacactgttgtcaacctgctggaaggtattgaattccagaccgagtctgaatttgaatcccgtgtgcgcaccttccgtaacctggtagaaggcaaagatgacttctctgataaagtcggcaaagacaatgaaaaaggcaagccggatggcgacaagtctgaaaaagacatcaaagaaggtaaaaaaccgaagaaagaaggtgaagccgacgatgatgacgatgacgatgatgacaaagacgaagttggcaaagaagtcaacgaagccgtccgtcgccagatcagtgctttgctgaacggctaattttagcagccacagccctcgaaagggggcttgattttgaactactaagtaatttcaacttaactgaatatcaatgtaaggaacgagcatcatgactaagaaacttgtaaccgaagaaatgcgcaaacagtggctgccagttctccaaaaagaatctgaagctattcaacctctgtctgccgaaaacgtaaccatccgtctgatgcagaaccaggctgaatggaacgcaaaaaacctgggtgaatctgacgcacctggttctgtgaacagcactgtcggtaaatggcagccagttctgatcgacatggcaaaacgtctggcgccaatcaacatcgcgatggacttcttcggtgttcagccgctgtctggtcctgacggtcagatctttgcactgcgcgctcgccagggcgttggtgacagttccaacaccgcacagtctcgtaaagaactgttcatgcaggaagccgattccggctattccggtgatggtactgtacaggctggcgacccgtcaggctttagtcaggctgagatcgaaggttccggctctgtcgtgaccactatcggtaaaggtatgccgtcaaccgacgcggaactgctgggtactaccaccaatccgtgggcgcgtgttggtatcaccgttcagaaagcgaccgttactgccaagtctcgcggcctgtacgctgattacagccatgaactgcgtcaggacatgatggcaattcacggcgaagacgtggataatatcctgtctgacgtgatggtaactgaaattcaggcggaaatgaaccgtgaattcatccgtaccatgaacttcagtgctgttcgcttcaaaaaattcggcaccaacggtgttgttgatatcgcgcaggacatctctggtcgttgggcgctggaaaaatggaagttcctgactttcatgctggaagttgaagcgaacggtatcggtgttgacacccgtcgtggtaaaggcaaccgtgttctgtgttctccgaacgtggcatccgctctggcgatgtctggcatgctggactatgctccggttctgcaggaaaacactaaactggctgttgacccgactggccagaccttcgctggtgttctgtccaacggtatgcgcgtctatgttgacccgtatgctgtagcagaatatatcactctggcatacaaaggcgcaactgcgctggatgccggtatcttcttcgcgccgtatgtgccgctggaaatgtaccgcacccagggtgaaaccaccttcgctccgcgtatggcgttcaaaacccgttacggcatctgtgctaacccgttcgtacagattccggctaaccaagacccgcaggtttacgtgactgctgacggtattgctcaagacagcaacccgtatttccgcaaaggtctgatcaaatctctgttctaatcgacaggttttgataacaaaccccgccgaagcggggtttttctttatagggatatgtaagataataaagcctcatttatcaaaggaggttaaaatgtctcatcaattatctggcggtgcagtcgatactctattcgttcttttctggtttggacctcgtgaagctggggaaatacctgctaaatctggagaagccgaattggcctccctggggttttgtaaacgagttgatgttaaaaacgtaccaaaaggtcgagatacacatctgtgtgtactcaccgaggaaggttacaaatacgcatatctgaaatatgtttctcaaaatacgtgattgtttatcgtaacgatttaatacgcgaatagttcaataacaattcctgttgtacgatataactttcttacaacaggagtatcagtcatgacaacccgaaatattacctctgaacaatccgtgtcacaaatcctcattaattcccgcacccaaaaagaacgtctttttgacatcctcactaatgtgatcaatattcgtcgacaattagatggtgaatttcccgaatctgattctgaagaaaaatcaacacacaatccaggtcttgttggtgctatccttaacatacaaggagagcaggacgatatcatcgatagaatcaatgcccacatggacgtcatccatactaaactgggaacgttttatcacccaaattgtgaagacgagtctaaagataattgttaatgttttcaataaatgggtaatgaaccatcattacccaactttcacaataggaatacaactcatggcaaaagaaactcttaacaatgaaaactccaactttgcagaatggcataccgaattagagcgcgttgcccgttctcaacatggttctgccgctgatgcagatgcttggcgtgaagattatgaatctggaaaaactccacgtcaggcttgggaagacgaatgggggtgggatgatgccgactctaatccgtaagatatatctggataagaactatttggaatctaaaatcgaagttttcacggctcaaaaaatatttgaagacggtgaaacgagatattatctgaatccagacctttatctcccaggaagtaatttgaatcgcgacataacccaaatggacggcacgatcattcgttgggaaatagcgacgcaagaagacaactgtcacaacataacactttgaagagagaactacatcatgtctaaacaaaattatatctttattagtccaggacgttcccagttttttcgtagcgacatcgatacacctatcgaaggttttcgcattttcaaatgctataaattggccagcggtgacatcgattatcaaagtcacgacaatgaccgccaagacttggttgataacggttgggaagaaatagattctcctcaatatctgaccgtcaacctttattatatgaaggaaagcggtaaatattattcgactggggagttaaaaatccgagtttcagaagtccaatccaatccggcaaaaagttggtatgacacgcaagagttagtgcgtgatctcctgaagaaaggcgaactgcctggcttggttaaaggttcccgttttgataccttcatgtcctgtgaacaacacccgtgctatgttccgactttatttaaaatcagcaacatgtgagagattaatatatgagcaatttaccactgcttcaagtcggcgatgtgttcgttctcacagaagaaatgaccattgaaactcaggtgcctaagcatttcttgtattctaattgcaagggagattggcaaacggactctggttatatcacccctgagggattcttcggttacatgcaagggcattatgtcgttaccaaaacagctctacaaggaggtagcgctggtcacgacccatacccagacggtcatcatgtttgggcacagcataccgaaaatgaacgcatcaggatctgtttttatcagacaggcagcttcagaaacatgcaccgtaacgttccagtagtcggaaaagccaaggcgcaaacatggacttgggagaaattatgagaatcaaaaagattggagcaagccctttgacgggcactatcttccagggcactctgaataccaaaaccagtatgtgggtaggtgagaagaccgacgtgactaacgactgtgtagcggctacggctgaacacatgcgccacatcaagaaagactactgcttccctactaaagacggcaagttcctggtgctgagcgctgaggttcacgacactcttcctgatcgcttcaaataaatttaaaatttattcaataaacggggtttacttcttcaataaaccccgtatacttcattccataagcagcgcaacactgactctgaaatgaaggaactacatcatgacaaccgtaaaaatctccatctgggcattcaacaatttcaccaaattcgacgcttttgttaagaacgctctgggcatcaccaacatagaatctttcacttcttatgaccgcgaagatcaggtcaaaatgcttcgtcctgaaatcgaccttgacgcaatgctgaacgctatcaagaatcacgatgacaacctggctgaattcgaaaacctccgcacctctatcttcaaaaagaacggcggcattcgtaagctgagcaacaaaaccatcaaatatgtcaccgaagggttctttgtggtgttcgacttcatagaaaccaagccgaacgaacttcgtaaagatttagacggcaacgttatcaccctgaaaatgtttggtgaagcaaaggccgatatgatgcgtgacggcctgtgtgatgagacattcacatacgacgaatattgcgatcagtgccaagatctgtaatttgttctaaaccatttgaacgggtatgttaaaatcatacccaacccctacaaatagaggactataacatggcttgtaaaacaccagaaatgaaattagttgtttcttcaaaggggcgtcgcagccgcagtcgaaatctcatcacccgtttagatattctacgtcaaaaatatcaaggtcacagcgtgttcacacgcatccgtaacgcattaaaggaagggcgaaccgagttggagctatatcgtccaaacggttcgactcgtgcgtatcagaccaccgacggtttgctggaactgatccgcctctcgggaatgagcatagaacctcgttcatcagggactcccttgtgcagcctgtatgtaatcggcaatcttggggcgctgtgagttctatgaagaagtatatcgttcttataacgggttcccgttctataactgagcgggataaaatcttcgccaaactggatgaactgttagatccccacgagatcgaaacttttatagaaggtgaagcggttggtgtggacttgatatctcgtgattggtgtgaaatcaattatgtccatgttacgccaatggggatccctaaggattatcacacgatatatggggaaggcgcaggaaaccaacgtaacaaagatatgttagacaaagctacaaccctggccaaacaaaaaggtcttgaggtctttggaattgcgttatgggatggttcgtctactgggacgcaagacatgattacccgcatgaaaaaagcaggagtcaatgtcaatatcactcttatgggcaaacccaaaaccaaacgattgttataaatatgtttgccttcacacccatatggagttagctatgctgcaattaattttgattctttgcgtcgcttttaccatgtactttctggcgcgaatcagttatatcacactaaatgatctgaaaaatacaccacgcgaacctaaatttatatttctaaagaaatacaccaaaaagttctgcgagaattttatctatcatcttccatgattataaatacccccaaaattgggggtattttatgaacgcacaaatctacgatgtcctttcctctggtaacgcatttagcaatccgcttccgtccctctccggtccctcacaggatttaatcaccagtggtagtaccaatataccattgataactgccaacgcgactccggaaatgcagttggctatgtcagcaggtggattaacaccagataaacttacagcagccacaaacatgtatagctctgccaacacggggataacgacactgaacacatatggcgaccagagcataaatgaagcgtattcaagaattgggacttccgtttcatataaatcaggtttgaaaagtatatcacgggaaccaaacaattgtgacctgatcaacaaagcattcggtgttgttcaggatctaggccgccaatggttaaacgctatggaaggcgctctccagactgtgactaacaagatatcggaattgtatgacatgattatggaaggtgcctcagaaggtatggcaaaactacagcaattggctgctgaagtaacaggacatatcaatgaggcaataaccgccgtcaatggagttataaacgatatcaccgatgggatagctgcagaacttgctcatattgaaagtatgatcaaagaatgtctaaacttctcttttgcgaatgttcttggagaatgggccaaagatctctgtgctggaggcgttattgataaaatagggactccagaattaaaagagtccctgaaataatcaatgtctagcggggagaactcccactggttcgataccttccatcaccagggagttcaccccaacatcactcattcgaatccatactttaccgcctggtcttatttcatagatgttcatttcattacccacagcagatccattaacgaaaacaccctgagtggcaattcggatcggagatttggtgttattaactatctgaaatgatattgcaacgtctctggtgatttctacgtcccttgcttgagtattccttctgagtttgtcaaagaaagaagggttgaaagtcgctatgatgctattattctgagagtaataaatacttgcggatagatatgggatctttccttcgtcacctttcctcaataggggaatcgtggttttccagtatggtctcagtgactggttgttatcatcaaaacaaccaacagcttgaagagtctggaagttctgaaaatcagatactacttgattcagaatatttttattgcatttatcaatgtcggctgagatgttaacaggtatggttctgatttcaggatcccagaggtcggttacagaaatgacggacgtgacaaagatacggcaacttgtgagtgcaagcaccattatcatcgtcatgatccattttctgaatccgcgtgtcaggaacactaaaacgttctccacagcaaaggtgctaacatgacaggtgtacaattcaaaaacgaaaatcctaatttcgcggcttccgataaatggcgcttaactatcggagacctgatattgcttactcgttcaatacatgacttcagtattcctggagtgtattctgaaggtatcgagggtccatcccctggcgatgtgctcaactccataccttcagaacgtctcacgtatgaccccatcgtatttacgttcgttatcgatgagagttggtacaactgggaacagatttacaattggattgcgtctaatgctggatctgatttagctgttagccgcgacatcaccatcgaccttcttgataacatgaatcgacctattggtctacaattgttgttggaagaagcccgtcccacagctttggataacgtattggtggatgtggatgctgcagttccccaactggtcacgactgttacgttcaaatatttgaaactgactccgactcgcgtcacccgcgaaattgataaacaataagttataatacagagagttgaaacagcagggaataaatcatgagcgaaacaaaacttgaaacgatgaaaaccgaagatataatggcagaattggaacctcttatttcggtaaatcctgaagacatgaacttggaccaaatgtcattgaaaattggtcgttcgtggatgactgtgcaacgccattatattcgtgaaggaagatatctggagtatctaactggcaaattccgccaaatagatctctatttacgccgattttatgcaggggaattgccgcccaacgtttatgttgaacgcccattaaaagttcgacccctgaaatctgatatagacgtatgggtaaaagcagatgatgattatatcgaattgtctagcatgcttcaagagcagaaagccaaagtcaagttcatcgaaagttgcttagatcgtttgaacaaactcggatatgaagtgaagaatgctattgattggcgaaaatatttggatgggatgtaatggtttctctacaggctttgcgcaaacttaatgactttgaaaatcgtcatcatacatttcagcaatattccttgccgttcaaggaactgacttgcgtaactaccgaaaatggtcgtcactatgtttcccccactggagtcaaactgacttcagtgacgaccatgttagggcgtactggtgaccatacatggttagaagcctggcgggacaagttggggcatgaagctgctgatatagaaacacatcgttgcgcagaccgtggggaaaaacttcatttggcttgtgagttgtatctcaaaaacagaccaatgaaggaagttctggaagctgcaggggaatacgtgtttatgttcaaacaggttttcccctaccttaacaaaatgagcaagatatatgcacaagagattccattatatagcgaagtcctgggtcttgctggtcgtgttgacctcatcggggtttacgaaggaattccaagcataatcgatttcaaaacatccaacaccctaaaaactcgaagcatgatagaagattatagtattcaattgtgtctatactcagtcatgtttcaacaaatgtttaatgtcaagatagagcgtttaatcaatattatatccaacgaaaatgcccttgttcccaccataatcgaattcgatcgcaaagacgttatttctaaaatgtttgaccgtgtccgtctgtatcacaaaatggacaaagaacagaacggaatctgggcggatcgctaaatactccataatattgggggaaattagtgatgtctgagatccaaatagtcaaagtgaacgaagttagaatgcggatccttgccgaggattatatccgtgaagaactcaacgattacttcaaatttgaagatcctaatttccaaccgaatccgttcagtaaatgggacggcgtagtgcggttgttcacaaagagttctgggcttatcgacatcggcctgttatttgaggtgttcaaattctgtaagatgaacggttacaccatcaaattagatccggccttaaaatatattcaagatattccggatgaagaaattcatgcgttcatcgatacattgaatgcggtgtaccgagacgaaaacaaacaatatcaaaaagcgattgtccgcgattatcaatttgattccgttgccacagcaatacgccaaagcagatgtgtcctcgaagctgctacgagtgctggcaaatcctttattttgtacatcatggcgcgttactacaggcaacgcagagatgccctcgaaagtaatctgaggacgttaatcgttgtaccatccattcatttggtgactcagttgtatgacaacttcgaagaatatagccacgggacagaatggaagccattgatgaatgtccagacaatatacgaaggggcaacgaaagagattttcaaacccatagttatttcaacttggcaaggtatccagaaccaacccaaagagtggttccatcaatttggagatatcgtcgtcgacgaagtacatacatcaaaatcagataaattgtcttacatcttgaacaattgtattaacgctgatcaacgtttgggagtaaccggaaccctggcgaatacaaaggtcgcagggttacaagtcgttgctcactttggtgcatatcataaaattattacagcgcgcgatttaatcgatctaggatatgcggcagacatcaatgttaatatgattgaagtcatacatcctatggcagactgtattgaaatgcacggtgctgagtatcaaaaggaaatagaatttctcattgctaacgaacgccgcaatgaattgatagcccgaatggctctgtctttgaaagggaacgtcgcagtaatgttcgaacgtatcgatgcacacatgatgttggtttatgaaatgctatcggcagtcaagaaaaacgtattcgtcatcaacggggaagtcaagacagaagttcgtaaacagattcaggctgctatggaagaaggggaagatataacactgttggcatcatatggtactatgcaacagggcgtatctatcaaaaaactccatcatctgatattggcccatcccagcaaatcttatatccgtgttatacagacattggggcgtttgatgcgtcagcattcctctaaggatgttgcgcgaatctgggacctggtagacaacctgcgctacaacggttcctacaaccatgctctcaggcattctcatgagagatataaattttatcttatggagcgccaccctgttaaaatgaaaaaagtggcgctctaaccatttcaaagatcttgattcacataggcgggggttgcctcgccttctttgttggatccaaataaacagtcgatcgtgccaatacattgttgtagcactttcggctcaagaatctgagaaaacaattcttcattagatgccagcagtggaacaactttcacttcccgcgcctggccttgttgaataatgccaacatacagacctttgttatttgggagtaatggtatcaccaacatcggtgacacaataaaccccgatgtacagaaaaggctttgtaaataaacgccttttaatgacggacgttgttctgctatggaaagaattgcttcttggtcttcccctactgcgtataaaccaacaaattgcttcatatgtatttctcgcgtgttagcaaacttggctacataataatgtcatgctctgtttagaccaaataaaatattttaattgttgaataaaaatcgctttcttgttgaacaatccgtgcgtaatatacgagttgtgagctttgcgagcaacgagtattatcacgagcgaagcgagtagactgttaaagaaaggttttattaaaacagaatattataaatgccgtcaggcatttatttatcgcgcgtagggcaatttattacttgttctataactacaaacacgcgatactaatttgcgtcaaatatgacttgaaccccactatattttgaggatacaactaatgcaagagatcactctctccgaacgcactttacaactgctggctaactttgaaaccatctgcccctctatcgttctggcacctggtaaaaagttgcgcactgttaacgattccagtacagttatcgctattgccgacatcgacgaagacttcccgttcgaattcccgattctggatctgaccaaattgctggcaatccagcgtctgccgagttttaaaggcggtaaaattgaaatgtcagaagaccacattttgctgaaaggtgaaaactcccaactgcaattctggcgttccgctaaagaactgactgtcgttcctgcagacagcatcgatcttccgtctgtagaatttgaagcaacggtcactcctgaaaaaatgaaagagctgactcgcgcctgttctactctgggtcacaaaactgtacgtctggttgccagcggcggtaaaactcgtctggtgggcacgaccactactatcgataattccaacgattacactgttgagttgggtgaaacaactctgggtgattttgctatgcctgttgatgtggtcaacctgaaaatgatcgaaggcaactacgtgatccgcgcctgtgccgaaatgcaactggttaacttccaatccgcagacggcaccatcaactattacgtcggcatgcagctggattaatcaataacgattcggcgctttattatcggtggcgggggtttccccgccctttgaaacgttttatctaaagaggctgcaatgtctaacatcactatcgtcacagatcaatacgcgtgggaaaacaaatatcgtcctgacaatctggatgagatcgtcctcccttcagacgtccgtgcaaaaatccatggctatgttacatcaggcaatggaaacatcccctcaatgctgttctattcccctcagcctggtacgggcaagactacttctgctctggctgtgtgtaacgaaatcggttgcatcaaaccgttgatgatcaatgcgtcactggataacagcattgatgtcatccgtgaccgtgttattcaatacgcaactggcgtcagtgttttcggtggccgcaaagtcgttatcctcgatgaagttgagcgactttccatggcggcgcaggaatccctgaaaggcatcgtagagaaagtctccaaaaactgctccttcatcctgacgactaatgcgaaacagcgcgttaacgaacctctgcgttcccgctgtcgtgaaattgattttatctggtccgaaaaggaagcagatgaagttaaactccaattcatgcgtcgttgcgcaactatcctgacagaggaaggtattccgtatgaagctggtgtaattgcttccattgtgaaagcatatttcccagataatcgcaaaattatgggaactcttcaggataatgccaccacgtttggtaaaatcgatgaacgcgctctggcacaggttaaagcaggggaattccaagcgctggttgatttcctgaaaaccaatgattgggctgggatgaagcagtgggtgaccgacaaccaaaactacatcactgaagatttttattcccgtttcttccatttctgcgttccccagaacaaggaaaagactcctttgattcaaaaagagtcaatccctgatctggtatgtgtctgtggtcagtctcagattgaacatcgttccgtcggcgacgtctggttacacggcgtttacttcttgactaacgtcatgctcaacatcaaatggaagtaatattatggccgctccgtcactgttcgattacctcggtgcgttgaatagcaccaaagagaacctgttgatgtccgaagaccccgaaatcagaaaggcatttgatcctttcatgactcggcgcggccttgctcagagtaaagacacgcttgtggttgctgaacaaatgaaccgcttccatgccattactccttggatgcaatggaacctggcgtttcatagcattccggcaaaacgtcgctatggcaagtggtctaagaagggagctatggatcctgatgtaaaattgatttctgagtattattacatcagtcctgaaaaggcttcagaatatgttcgattcctgccgaaagaagtcctggctgaaatcaaggcgaaggtcgaacgttccaacagcaacgaaaaagccaaaccgcgcaaggctaagtaattcaaataattgttaaaggggtatgagtcatggcgcgtaatacgctggacatcttaaaactttcagctgtcaacgatgaggccagcattgttgactgtatggttgaagttcaattgaaccaagacaaaccagggattttcctgggtatcaaggaaacgctgagtcggataggggttaacactcgtcaagagcctaacaccttgtatcaaacgtgccatatcctgcataaatttggcagatatcacatcgtgcatttcaagcatttgttcatgctagatgggcactacaacggctttacgcgtgaagatgtcctgcgtatgaatcggattatacggcttctggagcaatggaatatggttaagatcttgcatccggaacaaataaccgaagtggctgacatgtctcggatcaaagttgtaaaacatgaccaagtttctgagtggaaactggttccaaaatacaccattcgcccaagtcgggcaaaaagcgaataactggagtcgctatattatgacaactacatttaataaaaacgtgagattgaatttggcttttggtaatgcggctggagatattacagcccctgacttcggtaaaattcgcaatcaggccaaactggttctggaagagacccgcgaattactggaagcggcttatttcgaccacgaggtgaaaattgagttcactgcatctccgcgagtcctgaaagaacctgctgttgtcgacatcaaagacattatgcgccagattctggacgctcagggcgacatcacaacagtcaatgacggcgtggcacacattgctggtttcaacggcgatgagtgcttgcagcgtgtttatgcctccaacatgagcaaatttattcgctcagaagatgaagtcggtccggcactggaatattattattcacgcggtttcccagatggccagttacgcgttgaaggtgaattccctcaggcatgcatcaaagtgaatgagactgtgatgtggaatggcaaagaatatccgaaaggaaaattcctgaaaaacatggctgtgttcaaagagcctgatttctctgatatgctgacgaataccccttcccgtcagatcaatgacatcgttgccagcatggcaattgaagaaggtcaggtatttgttgatgatctgaacggcgtggcgttggtatctccaaaaacgttggaagtaattctttctatggcagttgaaaaatcttctcgacccgtagatgttaacggccaaattgaaagttatgtgccagcgtatatcattaacgataccaatatgcctaagctgtgtggggtgtggggtgctaatgttcgcgtatacgcgtgcgcacctgttcaaaacactgtgacacacctctctcgcttcgattaatataatccgttattcactaaacaacagaggctataatggcctctgtttttgtattctagagaggataccatgaaagataaaccgtgtgacattccttattgcggtgttcgggaagctaaaattaaatctgcacatcccatgttaagcccattccataaacaactcgcatacgaatggatgagcgagcgttataagatccacgtgagaaaggacgtccagcgtcttcctgctccttggacggataatccaatactacggcaggtcaagttctgtaacgtccggagagagcacgacaggcagtctcttaacctgatcaataatatcgtcaagaacgacgctctgagcatggccgacaaaatgttcaactgtgttctgttccgcatgttcaatctctgggacccgatcaaggtggcgctcgagggcgctatgacgatttcagactttgctaaaatcaacctggacgagacccgcgcccgtcttcagaagtttgaatctgaaggtggtaagatcttcaccaatgcattcaacacaggcggtctaaaacaatgtctggcgttccccgaacttgttgtcaaccacaaggagcagcgcttcggtgggatgatggttaaggtacaccgtgttggccagggtgtcgtggatgaattggattacaaggtcgccaagaagctggttgaagagaatcctggggagtacacgattgagggttgggagccgtacatgccaatgcgtgtcatccgctctttgaaggcattcgtcaacaagtaccccgattacttcaatgatcttttacgtcttgactctccgctgaaagtttaccagcgcatgtatgacgacattgaagggcttgggccattcctggcatatcagatctgggtggacttcacatacatccctgagtatccattcagtgagaatcatttcactattgctggtccaggttgccgtgcgggtattgacctgatgttcctggataaggacggcatgactcatgaagaatgcatattctgggttcgtgatcatcagtatcaattgtttgctcagtatggttatgtccaagagcagttctggtcagccgaagcgccggaagatcagtgcatgaacgtcatgcagctggagaacatgttctgtgaattgagcaagtacacccgctgtgttgaggctatcgagagaggggaaaagccacgcggcaaggtcggttatgatggaggcgggagatacaaaaatcgatgcgatgaaattgtcctcaatcgcggatcggtcaatttattagatcgtttgaagaaacctgaataaagatttcttttacttcgttcttcaataactcttttcagcgtatattgacctcactttaacgacgcgccccgataagggcgctcactttgaactgaaacaggaagactataccatggcagaattcaaacccttcgcaacagcagttaataacaacgtgctggcgatgtccgcaactggtctgttcatgaccaatatcgataaagatgctctttgggatctgtatctggcatctttccctgctggtactaacccgctgtaccgcgaacgtactgaacacgattgtacctgctgtaaacaattcattcgtaacatcggcggtgttgtcaccatcgattcagaactgaacgtaatttctatctgggacaacattgatctgggtaatgaatatgacgtggtggctgcggcgctgtcggcgtatgtcaaacaacacgcaatcgtcgatgtttattttaacgataccaagaaagtcggtgttgagtcgaatcacgaaatgaaagacggaaatgtccgcacctacaaccactttcacaccgacctgctgggtagctatgttctgcgtccggacgcaatcgcttcgaaaaaaggtgaaatccgccaggctattgaattgtttgaacgcggtctgcgtgaaattactctggaatcagctgaaatcgttctggaactgattgaccagaattctctgtatcgcggcgcagaacacaaagcggcagtgaagggatttgttgaaaccaaaaaggcatatgaacaaatccccgaatcagcccgttctctgtgggcatggcatactggataccgttcaaacaatcatgttccacacggcatccgtaacacagttatcggcactctgctgacagatatcagcgaaggcgtggaactggaaaaggctgttaaatcgtttgaatccaaagttgctccggcgaactacaaacgtccgacagcgctggtttcaaaatccatgatcgaaaacgctcagaaagaagttattgctctgggtttggaagactcactggcgcgtcgttatgcggtttatgatgatctgaccatcaacaacgttctgtttgccgacctggctgctcagaaagcgatggatccgtttgctcagctggccgccgaagtgaaaactccgaccaagtccctggaaaaagttgaagagatcagcgttgatgatttcctgagcaatgttctgccaaaagcgcattcaatggaagtgcttgtagagaattctcatactagcaacctgatgtccctggttgctccggcgaacccagatgcgcctaacctgttcaagtggaacaacggtttctcctggtcttacaatggtgaagtgaccgattccattaaagaacgtgtaaaggctgctggcggtaaagttgacgggtatctgcgcgtttccttggcgtggcacaataacgacgacctcgatctgcatatgttcaacaacatggagcacgtttacttccgcgatcgccgctctatgacaggtgcccatctggatatcgatatgaacggtatggatggtattgatccaaaccgcgctccggtggaaaatatcatctttacagatgaaagcaagctgcgcgatggtgaataccgtttcgaaatccacaactacaatcaacgtgaaaaaatcgacgaagggtttgaagtcgaagtagaatacaaaggcgcaacgcatcgtttcagccatcctggcctgaaaaatcaggggcgtatcactgcagtcatcctgactgttaaagacaaacaagttgttggcattaagtccgaactggctaatgttgataagtctaaagaagtttggggtatcaagactgcgaccttccagaaagtacagctggtcctgaattcaccaaaccattgggatggggagcaaactggtaacaagcacgtgttcttcattcttgaaggctgtgctaatcctgaaggtactcgtggtttctacaacgaatatctgaagccagaactggataaacatcgtaaggtttttgaaatgttgggtagtaaaatgaaagtccagccgaatgaaaaccaactgtccggtctgggattctcgacgacgcaacgcaaccacgtgttcgtcaaagtgtccggcgcgtttaaccgtactgtaaaagtcattttctaattgattttgaccacaaattcgtaataaggaacaatgaaatgtttgatcaagcaacccgtttaaaactgcgtttcaactctaacaaaggtctgctgtctgttgagcaggtttgggatctgaacctgaatgccctaaacgaactggcgaaagacctcagccgtcaggtgaaggaagccgccagcgatgaagaagatttcatcggcgtgaaaagcgcagtcgactctcagttacaactgcgtttcgatatcgtgaaagcgatcatcggtgttaaactgaaagaacgtgacgaaagcgcaaccgccgccgaacgtaaagcgaacaaccaggttattctggaactgatccagcgtaagaaacagcaggagcttgaaggtaaatctcttgaagaactggaagcgctgttgaaataatctgtcggctccggcctgagcaaccccgccgtgtgcggggtttcttttaggtaaagaatatgaaacaagttatttgtgattattgcggtcttcctgccaaatacgtcagtggtgatgtactctaccctcatcgcccagatctacacaaactgaagttctgggtatgctcgccatgcgacgctcgtgtgggttgccatacccatggggatggtagacaacctctgggaaggatggcgaatgccgctctccgtgctgctaagcaggaagctcataggtcgtttgaccctatctggaaaagtgggcagaggagcaggagtgatgcttacagctggctggctcataatctggggatcaagaaacgcgactgccatatcggtttattcgatatcgacacgtgccgtaaagtaatatccgtgtgtaaccaactagaggtgtcaaacgatgtcaaaggaaatacaagaatcttttgaaaagttatctcaggtaatgaccgactgcaaccgccgaaattattgcgatgaagcgtatacgcaattgaaaaccgtcgtcgaatctgagttgaataacctttcaacttttaaagatattcagaccaacgtgctgtatttgtctgatgaagatctagaacattgcccccgtaatatccgacaattactagaccgtttagtgaaagtgggggtggataatgggtaatcgtgaagggattaaactcactccagaacttctaaagtcccacatcgccgaagtgatttatgaagaccgtgaagttggtggtcatcgtgtcattacatgccatttcaagatggacaatggattcgtcgtctgggggaagaatagttcaacttctatcgacccagccaactttgatgaagagttgggtaagaaactggcctatgaaaagaccttctcccagctgtgggaactggaagcatatcgcgcgatggtagaaaagcagatcatcgatgtgtccgtcaaagatgctaaaatcaacattgatgaatatgtgtcagaaagggaagcgatacatgagatgggtggttctgaattccttaagggtaaaggacaatccgtcgtgtttcgtcatcccaaggatccaagtcggggatttcgagttgaatttgttcgcaccgataatgaacgttataacgaacgacgtaatggtgaagcaagatctttggtggaagcgtatcttcgctcattcatgacttctgaagaactaaattctgaacttgtcttgcgtttggcgaaaacgtgtaatgctgcttcaggggattatccgtggtgtgacctgtcagaagacaaacgccaagcagcttgtgatcgtgttaagaaattcttgttagatccgaaagcccaacctggtgattccgtgggggcgctgtatagagccatcgtggtgtcctacttgtgattttcaaaagtctttcaaaatcaataacgtccggcaaattgctgggcgtttcaattaacaagccaaggtataatggtctaattgtaaagcgaggaaatcatgtccgtaaaaatgaaaattgatatgactaaaccatttttgcgttcaggagttgactctcttgactcacagtttaaacccagcgaagttttaattcaacagattgagactaatcgcgaattcatagaacacatgctggcgaagccggaaggtgaagccgtccataccactaaagaaggtaataacgatgattgaatcattaacatttaagaatgcggttgcgaccgtcgcgccgtctaaccatgtaatcgttgtggatatctccggttccatgtatcgttcacttccagaagtccgtaagcatctgaaggagaacctgccttccctggtgaaaccggaagatactgtgagcatcctttacttttcatctcgcggtgatttcggaacggtatttgccggacgtcagattaacagtgcaactgatctcagtgagatcaataatctaattgaccgtttcctgaagccttctggttgtactggttttgttgagccgctgaaattggctgcggaaactgccattagcctgaataaacctggttatgttaataacctggcattcatgaccgacggatatgacaactgctggcgttctaatgaaatcctggatgccgctgaagtactgcccaaagcgtttgataacattaccttcatcgaatacggttggtattgtaatcgcgaactgctggggctaatggcagaacgttctggtgcgacccacgtattcgccgaaggacagaatgaatatcagaccgaattggaaaacgtgctgaaaacttcttcaccgaaagttgtagtcgatgttcctctggtttatacccatgtcatttatgttgaaaatggcgtggcgactgttttagctgtgcatcctgatgaagagcaccctattggccacgtcagcatccctgaaagcgtttctcagttgtgggttgtaaaccccaacatgatcgaccagctggataatctggcagacatccaagcggcatacgtgctggcattttacggcgtgtataccatggacgcagatttggtttgggctgcgctgaagaaaacgggtgacgtccgcttcatcaaacaatatagcaactgtttcaccaagcaggactattccaacatcaaagtcgatctgacccaggctatcgtagacgaatctctgcgaggagttgatggcatcgattataatatggttccggctgaagacgccaccaccattgttgatgttctgacgtatctggctgaagcagatgtctctatcgtcaccaagcatccgctattctcttataagagcattgggcgcggtactgtacagaaagttgatgacaccgaagacaagttggccgaagagattgcgaatgcgacttctaaagaagagcgtaaagccctggcgctcaaactggctgagcacgaagattggacccccgaattcaatccagcagatgataaagggattgtcccgatcagcaatttggtctataattcagaacgtcctaacatcagtgtgcagacagttcaacacggaacggttgttgtgcctgagtttgtacagaagaaatatgaacttcctgaagagctgggaacctggcgttatcgcaattacaccattgtcaaggatggtatcatcaacctgaaaacaatgcctatcactgcagattctccgattgttcgggctaaggtggcacaggatttgactgccatgggtgttcatgtgtttggtgggccggaagtcttcattgtaaatctggaaagtgttccaatggttaaccgcgccatgacaaagaatatcagtgctgctgaattctttgcagacaatgtgcgccgtgaagcattgaaggcaaaacagaaagttttgaaattctaccgtgatgaactggttggtaaaggcaacgctactggtctggcttcaaaatatggcaaagaagcggctgatttcctgtcagcaaacggcattcgcgactatggattttcaccgaagacttcttctgtagaatcaaccgatgtctatatgagtcgtgaattgaatgtgaaaatcaaaggcgcatcttcactgccgagcatcgcgtctgttatcaaaaaacagacggacggtaaaaaactcaatgtcgctgatcagctgatcgctaatgccctggctgaatacaatgcgtttgttaaatcgccgatgatcaccagcgtaccggaagagactcagaaaaagctgatcgaaagttggattggtgatgcagcaaaagcagctatcaacgaagttcgctctctgaacaaaacgctgtccaaggtagtttatgggattgttgcaggccatggttggtttaccgacctggatttggaagaatccactatggatgtggaagttgatggggtgaaatacaccgtcactgccgaactggctgaaaaagaagtcaaaatctaaattcaactggcggggaaacccgccttcattttgagatatgcttatgtcaaacaaaccaacttatgttgaagtaatgcgtgttctggctcagtacgcttgtgacaacctgaacatggaaacttggcgcaacggggatcacaaggacaagtttgataacattgatgataatatcgatgctttagcgccgttacgggttaagaatgatctcggcggtgacgatcttgacatgattgagctggtcatggaaatcgaagaagagtacgatgtagaaattagtgacgagtgggtgggtaaacatggagatgatcctaccctcggagagttagctgaactcgtcgttgccctgcgcaagtaactgaagatccccaacaaacgttggggatttttacttttaggggtttacttctataaggcaaatgcgtattattgaattcatcgggcggggcgttcccgttcattgaaagaaggtatatattatgtctgctgtatcacgcgttgttgacctcaccccattttgcctgaacaatattgctaaccctaaattggtattgtttaaagatccctctaatttctgctggctggcaagttgtgatacccacaacggacgtcgtgctgtttgggagtttcccgcaactgatgaaggtagaatttccgctataagaaaattagaactggaaaattcaagccgagaaattaaacaggcataacttttattaaatcccaacaaacgttggggatttttacttttaggggtttacttctataaggcaaatgcgtattattcaataactggcgaacgatgggttcaccaggttgaaaggtaagattatatcatgaagaacaaaatggtagaaacttcagtggagattgatcgtaattggggcttgctccgcgctaaagggttccgtcgtgtgtatgcgaccaaagaagtatatgcccagcgccgcgatgttatcatcagcaagaagggaaaagattgggttgccgaagaagtcgatgtcgaaacaggcgtcgtcggaaaggtgaaagccaaggccgcgatcctcttcgctcttctgaagcacatcagttaattctataacttagggtggggtatgattaccccgcctatttgaaatgcaggaactataccatgaccgatcaacaaattcaagatttattttacctcgtccatgataagtcccatattatgcgccctggcactgtttctcctgtcgtccgtgatctggtgcaagaattacgcgaaccgcagatgaccacactttatcgtgggttgcctaaagaggacgttgtgcgtttagagatgatgtttgaacgaggcgacatgttccagacatgggattcttgtcttagcttcagtgaattgaagcatgtggccaaaaaattttctaaatcgtatggaaccagagctgttgctcgcatttgttacaaacatcgaaacggactcccagaggggcttaatgtctttggttctctagaaaaatttgtgaacaaagacgatgaatcagaaatggatatgctggacttggcgtccatggagaaagaatggatattctgccagcctcgcgtcttcccagtttcaagaaaaatggcaacagatgggacgatggtttttaccgtatcacttattactctataaacccacggcggggtatctttgccccgcctatttgaaatgcaggaactaatatcatgacagatcaaagaagtattgctcgttcggaaatcaaagatattatacagagcaaaaccaacgaaactcatgctcaatccagaatgctatctcgatttgttgaaattaagtttgacattgaagagaataacaatcggagagcgctggagggaatgttggcgattatcttgggccatgtcaccaattcagcatacgacatggatgatttaactcgtttgttgaacgcccagaataacgttgggacttctgttcgcgaattgctgtttaagtccgaatgaactaactaagggataaccctatatcccgaggtgtgtcatgttccttacatcctttattgactggatgaatatcataaatggtctgaggatccctgttgggaacctcgggctttctcgttcttctatgccgcaaatcgatgccgacaaacaagaagattttcacaaatatcttgaagacaacggcgtgtccatagatgctgcgcaagtacccatcaaaatgcttcgtcttacccagaacgaaatcaataaaatgaaggtctggaagattatgaagcagatcagaaataagaagcccatgggtcgagtttgggtgtcttctgacaattacgttgttgacgggtctcaccgatttgtcgctgcgttgaacatggacggcaaacagcgcatgaaggtctacaaagttgatttgccagcgatggaatttgttaaactcgctaaacagtttcagggtgtgcgatacagaacggtcagcgatacccgtttctgaatacgatagtttattgaaaccctttactcaacatggtgacatccctatcattttatccaatgaaataaggagaatcaccatgaaacgtttaatcatcggggttgtgctggctggtctggcacttcctgccaccgccaagctgaactccacaggtgaaaaactgtggtctgatctttcctactgtgctgggttctcccaagctgttgctatagacaaaactggttccatagagaactttgctgaactctgggccacaggtaacgtcacagatgccgttgtaaacgctggtattgagtttaatcgatataagcaaggcgcgttcaacctgaaaggatatatgaacgacgaagcatttaaccgaggaggcatggaggcgagcgatcttattatgactggtcgtatggacacacaaggacgaatgaccgtaagacagtgccgttctctcccttctccagcattacaaaaaggtgtgcaggttggccgacgtcagctggacctcatcaatgatggaaaccaatgtatccgagtatttgaatatagcgctgatcaagaatctgatcgaagactgaaaaaggaatggcggactcgcgcccttggcctgagagcatggctggtggaaaatgatcattattatgaagaccgtgttcagaatggtttgaaagatctttccactaatttgtctgtcaatcttgatgatcccaggcttgcccgtgaactcaagcaaacaagaattgactgtgataatatgatgaaggagaacgaaagatgaaaatcaaaatcttattgttattcctgtacactttcatgtcttcccaagtattggcagctccggccagtaagttgaatatttgtgctgcttggttgtctacatatcaacaattccatccggatgataaggatacatcaatccttttatctgattatgaaggtgaattgaaacgccttgatctctatagtgtggaccagatagagaatgcgcttgatttacccatgatggaatctgctgcagacaacagcaaacagactattaaagatttgaccatgtgccgtgaaattgcaactaatttcgttggaaatcaaagatagtttcaggttaaaattttattccccattgaactgaaaggtgtatttcgtgatgatcattaccaaaaagatcgctgaggcgatcgttgaaaagaataaaatgttcgcacctagcaaagcaaaggttattctgatccatgatgatggtcgcgtggtcttcgctgaaaaatttgagcaagggtgcgctttctggtttgcagacactatgcgtgaaagcaagctacagggcatcatttacaaattaactctggttgccaagcgcaacgatcagggtgactgggttacggtttgtgatttcagctccaaggttgatactcgaccaaaatctcgccaagagagaattctggaagctcaggaaaaacttgttgctgctcgtacttcaccagtcattaagccagcagtcatctccccttctattgatctgccgcctgtcagccaaaaggctgttgagcaatatgcagaaaaacgaaaagaagaacccgtgaagtctccagttgagaataaagaagaatctgaatttatttcaatcaggcagttatgtccgcaagaaagacgattttcttcaccaaatgatttattcgtgtctttccggtctaatgggaagattatattgtgcaaaaccctacgggaacaattgcagtggactaacatcaatatgatggtgagtcggaacttcaaacgattcgcaatcggtgaaggtaaagattatcctgttaataagagcggaacatatgcaaatcgtcatatgtgcgacaaattgtctttcccaaacgattccggaaccatccgcgttcgtttagaatgggatgaaagtttgaacatgtatgtcggtgatataaaataattcaataacattagggcggttattattaaaccgccttttatttgagagatctatattatgaacaacaagagccgtattatcatcggggctgaccagggtaatttatattttgaaagatctggtgttgtcgcccacgctatccttcccactggaatttccgtttattgtgtggaacatcctgaccctgcagttaaacgtctttgctatgtctcccgtgaaaatggactaattgtattgccagaaggcgcgattatgcgcgctcgtttttggccgatcgattttaattccatgagcaatagggagatcgacgcccgtttcttcgcatataatatgaaccggactctgaaaaatgaaactcagagagttgttcatttcgacaatgaaactcatggagtgacgttagaattcagaactcgtggtgaagctcttcaggtgaagaaatcaagctggaaaactgaagggacgttcacatatgattatgcgttaatcgacaaattgtgtaatcagtttcgcattagcctgactgccaacgacgatttaggtcagtatgttgtatggacagcccgttcaccacatatgaacggtatcatgatggagagtgaaactcgtaatcgcgctgtgctccaactgctgctgttagacgcgtattctcgcaacccgttggaatatctgtctcgggaacagattgataacgcgctttctcattgtaaccgtctggaggtgtaatatgtccggcgtatggtgtgatgtattgttggtaagattttcaaacgggatggataatcagatcatttcattcttttatcctgttcccttttcttctatgggtctgactgtcaagcgcgttcttgacatagaagaaaaactcaacacgatgccgtatatggcgcgaccacacgtgctgggttgtgaactgttaaacgaaatgatgattgacgagaatcttgttgaaacaattatcacgcgtgaacagatgaaaaccatactgtctcagggagaaccccatgtcgaataaaccatttgaacttaaatttcgcgtttggcatattccccaggttcctgggatcgcttttcatgttgaggttccaacatttgaagaagcgcagcgcctgaagaatactctggctgattacgacctgtttcagtataacaataacatcaagccggattattgtaatgcttccggcgttcaaatataccaacatgatctgactgaagaagatctaaatgatatgggactggatgatcgttgggttgatatcgaagatattgacgaactcaacgattattttgatcatctgcgtagcacagggtgggaaatatgatcgggttaatccgttatctgttcatattcgtaataacaacattcattttatttttctttttcaaatacttgaaagagactaaaatctaccggagtaaacgttggcgcaaacgggtgagagtaactttcctactgctgatttcttcggttacaataactgcactcacattgggtgcttatatttacatttttgaaggagtacattgatgttcaagaaactggtatttggcgcaattatggtcctagcggcaagccttctttcaggctgcggcggtgttatcgatgaaggaaacgtcggtgttcgaacccaatggggtgaagtggacatgaaccctgtaaccgcaggtatctacaccagtttcgtttcaagtgtagatgtctatacgactaaagaagccgtcgtgagtctgacgaaaatgacgccaaaggccaaagacaacctgaccttggaagatctggatgtggacgtttattacacaccgaacgtcgcgaaggttccttggttccataccaaatttgcaggtcagagcgctgaactcggtgacggcacgatagcggtgggattcaacctggttaaaaccgccgcagcatcctcaacgatggacgccgtttcttctttggattctatgacaattcacacccaacgggctgagttagaaaagatgatcgaggaacgcacccagcagcaattggaaactgctgcgcctggcatgttcacaatcacacgtgtgttggtgaaaaaggcgctgacagatccttcgattgagcaatctatccgtgataatgtcatggccgacaaacgtttggatacagcaagaaagaacgttgaaattcgcgaccaagaagccaaggcgaatcagaaactgacgacatctctgacacctgaatatttacaacacgaatataacatggtgttgcaatcttgcgccaacagtgggaaatgtactctgatcgttgacggatccggctcaggtaaaatgctgaacgtcggcaaataaacgctttattcaataatatttgttaggtaagctgtagtcctcttgtagtgaaacatcgtaaacaaccaactggagtaatccgaatgaaccgtactgattttatcgctcacatcgcccagactcagggtatgaccaaaggcgaagctgagaaaatcgttgcatcttttatcaatggcgtgaaaactgcagtcgcggctggccaatctgtccaattcgtaggcttcggcgcgttcgaagttaaacatcaggaagcgcgcactggccgcaacccgctgaacggacaacagatccagatcgaagcgaaaaatgtagttaaattcaaagtcggtgaaggtctgaaagctgctgccaacggccagtaatcacatccagagcggtcgcgcctagcacagcgtgttaggctaaaagagaaagccgaggcattaacctcggctttttcgttgttgggattcgggtaaactataacagaatccaaaggaggaacaacgtgacagcatttaatcaacaccgaaaggcgattgacgccataggccgtaaggttcctatggaagaaatacaacgccttgccaaagatgaattcccatatgacaaagcgaacccaggccagatggaatgcatcgtcgaagccatagatgcgcttatcaacaaaaaggttaaacatgtcatcattgaagctccgacaggtgtcggtaaatctttgattggcactaccattcataaagtgattcgtcatctggttttacaggctgatccttatgggcaatttcgcacttctatcagcaccccgaccaaaggtctgcaagatcagtatgctgctgaaaaagcagtatcgatcgatatcctaaaaggcaagaagaattatcgttgccacgtacatccagatgtgtattacaatgctgtccaatgccgtatagcatgccgcgacgggcattgtagcaaacgccgttgtccttatgtacaggcgcgtaatttgtggacggatatttcatcattgcgttgtactaatgctgccatgatgatcgaaatgtgtacaaccatctgcatgaagcccgaaaaccgttccgatatgctgatccttgacgagtgccataaaatgccctctacgctcctagagcacacgatcatggaatacaatacaaaggccgtggaggggctgcgttctatccctgagggcaaggagatcgtctctaccattggtgatatcgtagaacgcaccaaggactatgtcctgggcaagttatattctttgtctggtgaactacattcaatgtttgaagacctccatctcaaagtagaaaatttgttggaagtccttgaagaattggtcgaagatgatcgcttgagtgaaagccaagtgatgaaactcgcagacattatagatgtcttgcataacttgagtgattattgcggtattatgtcacaaacccaagcaagtacatttattgtgcaagaaaagggggaagattttatccggtttaaacctgttatgccttctgatgtaagtgaattcgggttattccgaaaagccgattatcacgttcatatgtcagcaacgatctgtggtatagattcatatgctcgttcattgggtattcgacagggggattatcattccattcagatcggcaaccctatcccgattgaaaacagaaaagtcaactatatgcccatagtgaagatgactaacaatatgggtgactatgaaatgaaaaggttgacggaatatattgatgaaataatcgcgttccacccaggtcagtccggcattatccatacagtgagttatgatcgcgccttggcaatacaaaaattcagcaaatatcagaattttatccatgttccgcgtacccgaaaggcgttgatggatattatggaaaatgcttttagaacaaagtccccatgcgtcattgccagtccggctatggaagaaggttatgatttcaaaggggattacagccgtttccaaatactgatcaaagtcccttatgattatctgggcgatccactgatagcacatatcaattcagtggatccgtcggcatatttccgcaatgcagtactgcgcattgttcaaatgtgcgggcgttcggtcaggggtgttgatgactgggctgcgacatacatcattgattcatcctttgaatctcttatgatgcgcaacccagaattcttcccaacctggttcaccgatgcagtgtttgaggtttaataacccgcaataaacgataaatggcacttgatatttggttcaagtgtcgttatcgtgataatcatattgtccacatcttggctgaaagatatatttgcgatcggtttggtttgaacatgattatacaggctataaatcagatcttcaccacgataaccgattttgccatcagtgtcctgccaatgaccgagggtagactctcgagcgtgtactcggaattcgaccatatgaaacaatgcttttgggatggtcttcagtatgctggtcccaacaaccggaatatcaatagaagagtcgttggagtttccttcataaacagattcccaaccaaatattggatcacctcctgccacaggagttcttttccagtattggaattctgggttgcccgaagcgttcttttgtaggaggattacttcaccgccttcaaaattggaagtgtctatatcgtcccttgctaataaatctgccggagaaccaataaatttccaagtttcgcgatctaaaatagacttataataatcggtgaatacgttgctgtcgttagctgcattgattgcgtctttaagaccttcacctgtgaccatctcattaagttttagccacatgtttttgacagctgacgcagctgcgacctgaccttcagtcccacccgccgtgtagtcatatacgatgtccagcttttggatggagtcgtttatggacaagagcaaacgctcaatttctggggcggtcagactgaaacgatatgcgcgttctgacatgattcgttcctcatcgataaaaccagtttgggatacttatacatgattgaaatatacactgacggggcatcgtcccctcagaagacacgagccgctggttgggcatttgccatcagtccaatcactggagagcaatggaaagtgttttatgggtatttacccccgccgtcaacgaacaatatagctgagttactcggggttataaacgcgatgaagatcctatggaaattctctaattgcggtgaacgctgcattcccccagcccgcattatatccgacagccaatatgttatcaatagcgttttggagtggcgtcgtaaatgggaatatgaaggaatgccgcctaagaacacagaacttctgctcgagttgttcgattattatgacaaagtttgttcaatttgtgaacttgaattaaaatgggtgaaaggtcatgcaggaacgatgggcaacgaaattgcggatcagtggagcgttagagccaaagaagatagttcaatggttcttgaaaataaccgtttctcggtcaagaaagtcgttggttctttcaatgaattcattggtatttgaggattatcacgatgggtatgaattttgtagacaggggtgataacgtcaccaaatatttcacagacgaagataatgaccgtgttgttggaatattgagagattggattccggctcgcaaaaaggctttggccgaaggaacacccctaccaagaatccccaattatgtggccatgaatgttcagatgattattaagaacatgagtatgcgctacaactatcgtgattatccataccgcgaagacatggtcagtgaggccgttgtcaacatccttcgttacctccatacatttgacgtcagtcatatcggcaagaaaggaaaaatcaatttcttctcttgggtaacgatgtgtgccgaccgttcattcgccaagaaactcaccagtgaagaagaacacaattatatcaagttgcgttcatttgaagaagcaggtggtttcgctgcattatcagatgacccagacttccagcaacagacattcgtagacagcactggtataacaatggacttccgtgagcgtatcggaaattttgaaactaagaaagaagcccagcgcgaaaaggaacgccaaaagcaaatcgccatcaaagaagaggaaaagaataagaaaattcctcgcggtatacttcaatgtctcacaaagagtgaaaataccattaccgccgacgccgaagataattcagactatgacttcggctctactcaattcaatctggaagacgaaatatgttctatggaaccagattgggaagaagcaaagaaacgagcattagaacgggagcaacaaaataatggcgattgctaaaataggcgatttacatatcgggagccgtaatggttcccgttacgttcgagaattcataaagaattatctcatcaattattttattccagaattggttgacgccgatattaaagaagtgtggcaatttggtgatacgtttgatgttcgcaaattcatgtatggtcgcgataaagattggttgaaagatgaattgacgccagcgctgcgcaaagcaggtattaaatggaatggcatcgtcggcaaccatgatatcacgttagaagagtccaatcgcattaactggccttcttatcttaatgaattggcaccggatgtttatcattattacagcgaaccaactgaagtaatgattgaaggtgtcaagaccctcctactgccgtggattaacaaagagaactatgatgcgtccattaaagcgatacaagatacagacgccaagtactgttttgcgcatctggagttggcagggttcaaaatgtaccagtcctctgtatgtgataaaggtcagatcgacttggcgctcctttcgaaattcgagcgcgtggataccggacatttccatacccgttcgatggaaggtaacattcagtatattgggactccttatcaccttacctgggaagaccacaaagacgggacgaaccgaggattctatgtagacgacatgaatggcggggaattgtttatccccaacaacgaacaacaaacgttgttccggttcgtggaatacgactacacacaaatgtcttctgatactgaaggtaattggatcgaccccgattggttaaacaatgttttgggtattgaagggcaaatcgttaaggtgattgtacagaaccgtgacaatgccaagcattatgaaaagttctgtgacgcattgaagcgctgtaaatgcatcgattacaactttatcgatcggacgattactgttgcttccgagaaggtagaagtcactgaagagatggtggctactgatgctgtagaagtcttgaaaaatgatatccgcgcagccaacaatatccagcgccctgaaaatgtttgcaaacttgcagaacaattttataatgctgcccaacaacgtttgaacacattggatgcataataatgaacaatgtagacctattatcaaaatttggttcgctgttgaaagaagcggacactcctgttactgttgaagtaccatttgaaattcgtggggaaattacccacaaactgactttccatcgcggtcgcgctaaaaacttccgttccatcggtaatgaattcatggaaattgattaccaacgcaaccctgcaaccctagtcacatctgatgacaatggggcgggcaaatctacaatgcttgtttggttgttattcttcgttctgtataatgacacatacagcaagaaagaaaagaaagccggattagtcaattcccaaaacaaaaaggaatgcgtgggtgaagttgagttctccactcgaggaagtgaatggaaagtccgtcgtggtatcaaacctgatttcgtagaagtctatcagatggtagacggtgagtggaagcaggtcgtgaatgatgcggccaaagccgacatgaacaagtacatcgtgaatctcatcggcgtggaccagaagatgtttgagaactccctggttctgggcaaagagaagttcatcccattcacggaaatgtacactgccgaccgtcgcgctatggtagagacaatctgggatttggggttcttcagtctgatgaatgaagatgtaaaagcatctatcaagacagtaacgaatgaactcacgacggtcaccaatgattacgcctttcatgatgtgaatctaaaaggccagcaagcgcagttggaacagatcaataattccaatgcgatgatccagcaacaatctgcggatatccttgtccaagagaaggaacgtctgaatagtttgaattcagatattggtgcttctcaagaagaactgacggaattccgcgcccaagattctaaattagaaactgaattatcagaagtccgtaatcgcctgaatgccgaaatgaaaggggatatcgatgccatcaatgaagagtatgcggccaaaatccaagcagttcaggatgaagccgataccaaggctgaagattatgaacgcatagaagtatccgacggcgaaaggtcgttacaagaaatccgcgaaagaatggcggtggtcgcagaacgcaaaaacgaactagtcggtcagaggaatgctaatctcgacgaactgaataaagcgttggcgcggcgtcaacagggagagaacttccgtttcaaattcgtcacagaaatggaaggacatgaatctgccatcaaacgtttccacgatatgggcacttgccctacgtgtacgcaattagtgtcggacgataccaaatcacgcattgaaagtcaatattatccacaaatcagtgaactgactgacaagattcttcaggttgatacagccctagaagaagtgaattccctgattgagaattacaatgtcagagattcagaattatcttcccaagtttctgttgttgacaaagaattggaatccatacgcaacgaagcccgtgaagcagaaaccgccatcgcggctttaaagcgcgatatccaggggttctacgaagttgcggtggttgagaaagcatctttacagcgagaacaacaatctaaagttaatgatatcctcagagcagtagatgtccgctatgaagatatcacgacgtcaatccgtcaagcgcgtgagcagctctctaagagtatcaacgacacggctgataaactcaccagcctgaaatcacgccgtgcccctctggaggcatccattgccgatttagagcgtaaattggcggttaagccgacaccaactgacgccctggaaggagagatcgctcgcatcacggcattaatggaagatctgaatgcgcggcgcgtagagttggatgagaaattgcaagacctgaaccatcttctgttcttcctaaaagatgatcagaccaaggctcgaattatcagcctgtatttgcctttcctcaatagtaaaataaacgagtatcttgaagcactcaatatgttcttggatattgctgttgatgatacatttgaaatcaccatgagcgctgcaggtcgtaaagggcaaagcatcttctcgttatctactggtcagcgtagtcgcctgaacctggctgtgacattagctttacgagatgttgcaaacctcaaggcgtcggtgcaatgtaacctgtttgtgctagatgaaatccttgagaacatgagcgagcgcggcgtacaagaatcggttgagatgctgaagcataaatttggcgggaacaatctgttcgttatcagtcaacgcgagcaggaattccaagagtatttccaacacaacatccgttatggtctgcgtaatggcatgactgaagtgattaagaaggattgaacatgaactgggatcatctgtggttggctatcagtgtctttatgttctttgatgcgttgttgggaacgtacgagatgagtagggagtccagagtcggggtgatggtttgttgttcccttatcggcgtggtatccctttatttcgctattgggtttgccgcagagatcttcaaatgaaagttgtccacttcaagaaggaaccatacgacgtctacattggccgtcctggcaaatggggaaacccgttcgaggtcaaagaccacgggcggggaaactgtattgagttgtttgaagacgacttgtatgtgcgcctgattgaaggggatatcaccgaagatgaactccttgaattagatggaaagaccttgggatgctggtgtaagccgcgcccatgtcatggagatgtttatgtcaaagtgatcggtcgtattaaactattccgtaagttgggtaaatcttttactgaatatttgagacaaacatatgtcagaagagaaagatgaaattcagattgcttgtgatatcgctgtcgagttatggaatcagcgaaacaaacattatcaggtgacgttgaccccaaagcgttatctagagcaaggtggcttaatagtcgttgatgccgtgttaggtttcaacaaatatactgacatgcaaatcttggacggggatttcactattgcttccaatacagcttgggatgatcctatggaatttgtctacggttggttggaatgcatagaggaatcataccatctgatctacgaataaggtgaagggcactaaatatcacgtgccctttatcttttaaccctgtcaggaggctccagcgagctgcaagtcgacaaaccacataaggaacgccatcatgacgatgacaacgttgaaggccatggccttgtccgtgacctttaccattgccagcgggaatgtgcacgcttcccaaaatacaagtcaatgtgtttatgatttctctgagcaacaacttaatacgctggcaactgcctatcacataggcaagacgcaagaccttggttatactctggccgccatatcttggcgagagagccgcgctggtgaagatgtagtgtcaatgcgatacggtctcaaggcggccaacatgggagcctttcagaacaaggtgaagacagttggtgatcgcgagggttgtaagaatcgcaagtgctatgccaatgtcgccatcaagttgttgaccgaccaagaatatgccgcgcaagccgccctcgatgagatgcagttctggctgacctatcacaagcagaatgtacgcaagtctctttcctcttacaatgctggcttcgcccgtaatcccgcatcgaacgcctatgccgccgacgtcgtgaagaaagctaaatatcttcagaaatgtgtgtccttcaaagggcgttccatctctgaggtggatcctcaggtgatcgccatgaacaagcgcacgatagagaaactgaagaggacaagataatgaacatcactccatcccagaccggatatgaacatatcttggtcttcattgccctacagggtgcgaaggaaggcatcatcgtatccgagtattcaggtacggttcgccttgctgactacgtcgcgccggaggcattcattaagaagtgcattgatgcatggtgcctctatgccctcggcacgtatcctgctcctcctgatggcgagctgttgtttaccgtaccgactccggctgctggcgatacagacgcgatctttgccgatatgctggccaaggggtatctggtgtatgacataactcacgatgtctattccttcacctccctgaacactgtgtttgggttcaccataacctctgaagccaacatccctgactggggtatcttggcctggggtgttgacggcctgggcgcaacctacgcaacgtctgatgttggtcctgttctgattgatgagggtgtaaccatggagcagtaccttgctcagttcccccaacacaacatcacgggtagcgatatcattttggtccagcctggcgctgttgccctcccccgcctcgtggacggatggaagcctcagctgaaggacatcattgcagggatcaggtcccatattcctgtggtgaacccaacaccgtaaaataattcaataaaaggggctttcgcccctttacttcttcaatatccttccctatacttcattccataagcagtacgacaccaacttgaaacgaaggaactatattatggctatcaccacaaaaactgaattgaacgcgatcctgaaagaagctggcttcggtagtatcaaacgccagggcaaggttttacgcgatggcggtaaaatcgtaaagggtatcatgttgggaagtactggcccgttgagcatcagctcccctcagtatgacaatcttcgtaaactggcgcgtgaacttctccaagaagacctccatatcacgacgcaagaaaccaattatgtggttttggaatgcaacaaattcatcgtcagcctttacatctgcagcacccgctcatatccggtgtctgaaggttatgacccaatgtatctgacccattatgctcaggttactgtcaaagcaaaataatcaaattcaacggggagcttcggctcctcttttgttttcgctaattaaggaaaaccgcaccgatttattatcaggagaattacaatggccatcccatctttcttgaactttcttgaagagtctaaacaattagacgaagcgtttaactcatccccgtatgaattgacattcggcaagaagaacgctggtgacattttcttcacatttgttgatgaagacgaaaaagaattccgtattcaattctataccccacaaggtctaggcaagaatgttcgccaagtctttatcggtcagaagcgtggttccacatatcccgatgccatcggtcgtttcaagaatccaatgcgtgttatcgcatccatgattgaagcaacaaagcaattcatggcgacgcctttggggaaaaccattgacggctttgcaattaacttctccaagaaagcattggagcgtggtgttaccctcctgccgaagattatccgtcagtccggcctgaagcaaaagctcaatgtcatggacctgacgtatacccctatccctgatcgtgcattcgtatgggttgtgcgtaaaggcaaggatcctgctcaggtattcgatggtccgaaaatgaaaggtgtcacatgggatgatccggacaaagtcggtgacgtcccggttcagaacaatactccggatgtgggagttgtggctggttcttctgaaatcgaagtcaaccgcgatgacaacggttggaaactcaccaaagctagtcgtactcagcctatgctggtctggtctggtaaagaacgtgggcggactattaccgccaatatcgcacctcacgcggatatacctggggtgtatgctggtagtatcatgaacgagaaacgggtccgcgctcaatccccagacgctattgtacgccaactgggattaccgcagatccctgtgaatattctgaatgatttcaccaagcagtcgtccatcttctggaaagagatgacgggggattctggaaaaatagacatgcagactttgccaaaaacaggatctgtggatgacttacgaaatcagggttatattgctcgccttctggggattgtagacacatatgctccacaacaggtcggtatgtatgcaggtgagaaactaacacgaatcggcgcgggcaaatatcgtacttcgggtggtctggacattcaggtttatttctggggaattgtcaacggaaaactttctgtagaagtagtatcagatttgtctggtcatgaccaacttatgctggaaggagacgtgaaaaacgccgcttacgaaatagtgggcgcaatctcccgtgcagccgattctcaatttgctggagaagtagaatgccgtgtggttcctatcaatggtgccccatatccgattcaaaaaatttcccgttctcagggatattcttacgttgattgtggttctttcacattgaagatcgccgacgctctgctgaatcggtttaccttgggcaaggcggtcaaacttgatctgatgaaagagtttcctcgtacaacattcaaagtccgctatgattggcgtggaaaagatactctgttggttcaatcttccgacgagcgtactatctttggtgaaatcaacaccaattccagtgaaaatgcagtgatcactgcaaacatcaaagtaccaagcaacgctaaattttttggtgaaaccgttcatggcttctctgtagcgtgggatttccagttccctgctggtagcaagttgcaagccgcaagattcgttaccaacgtaacttacgattttgatcgaaacaaagccaacatcaccaccatgttgactgaaaatggtcgaaatgtagtagacagcaatggatatgatatttcgttgtctgtcaaatcagtgcaagatgcattggatgagatttctcgacgcctgaaagacgcggcttcgcaactgagccatctcagttcatcaaactataaccctaacaatctgttcctgaatgacatcactgtcagtaaatttggcgcgattatgttccaagacaaagatttggcacagtccaggaaccgcacggcagtcattgatgctttggctcgggcgaaagatcaggtcgctggtggtaagaatgcaggtgatctggccacctatgccgaatccattcgtgacagcgctcctaatggacgagatctggattggcaggtatacacagccagtaatggtacagttctgaatgttgcttgggatatcactttccgtcgcaatacgaccgaaggagcatatcgtgaattcaaggatcagatcaaccgtgccaaccaatacctgcaaacggtatacaacgacgcgaaatctaaaggctataatcctaccgagccaaatctcatgactcttgaacgcgctcgccaatccgacgaatgggctatgagtaatggtgatagtgcatacagtgaatatgaacaatcgctgggtggtaatcttcagattagtctgaaataagtcatagccccttcgggggctaaatacatcaaaatccttaggagatccacgatgaaaccgtttatagaatattttaaagaacaagctcaacaaccgcaaactcatggcggcatacaaggcaaaagtgtaaccatcaccaaacaggccgacggaacccaatggtgtactggtgccaccgtgacccagatcactccagataatcaggccgacatctataatctggaattgactaacggcaacaccaccaaagtaaaactcacaccggaccaggttcatgcagtttctgtcggtaaagatgtggttgccgttcatgacggttttgagttcttctttgggaaagatgccagcaaaggtcaggtcacagaatcctatgcccaagaattggcgaacatggataagatgatcggtaaagcgctgggcgttggcaaatctcgtaaaagcggtcgtacagagtttgaatattccggtattcctggtattggaaaacctgtcgcaatcgacttcggcggagaagacgatccatatgtaattgtcacagtcgctggcaaacagcatgacatcagtaaggggaaaaccctagctgcggacattgccaaatatattggcgttaaacctgtctaaggaggttgtatgaaaggattccaggacttcctcttgaaggaagcaaaggttgaccatcgccgtcttggacagcagggcatgtatgacatcaccgacgttggccgccctaacaaaggcgatctgattgattactatgatcgcaacggggataaacatcaggggaaagtgaagtctgtgaacgcacagaacattatgaccctgatgaacaccagcacgggtgaaaccgtcaagctgactttgatcaagccttgatgacaatccccctaaatacagggggatttctttttctgaggaaacaatcatggttaacgcgagtatattcatcaaccgtggactgcttgctatagcccactctcatgccctccacttcgttacaacgtcctatgccaagcacaaggcactcggggagttctacggggaactggaggacttgctggacacctttacggaagcgtatattggtgcgggaggtatatacattccaggattcgagaacatcaaattgtacaatcctgaaccgatatcgtatatcaacagcgtggtcatcgacgtcaatgggatctacaatcagtgtgatagccatttacaaaacacgcttgacgaaatcaaaactttgtgctatcaaacgatttacaaactgaagcaattgtcctaactcaataggatcatcatcaggttataattttgcccattcgtcatcaataggatacagataacaatgggcaaaatctttataccttctcactcagaaggttgtcaaatcgctgcaaaatttctacacaaacaaatgaattgtggtgctgtgttcgtagaaccaaattcattcgataatcgtgaatcccctgacgccatcgggttcagacctgggggttgttctatattaatggaagttaaagtctcccgcgccgacttcctgacagacaaaaagaaacctcatcgcatgaatccttccataggcatgggagcatatcgtttctatgtttgcccagcagatgttattaaaattgaagacctccctcccaagtggggtttgttgtacttcacccctagaaaatctttgaagcccgttcacgttccgaatatgcaatattcctctttgtcttcaccggaacattatgcatcatatttgtctaaatctttgggcaaaagaagacctacagatatccctccttatctattggggtacaaacagatgctggaagaattcgcccactttgaacgcaatcaggttgcggaacaaaatattctgtatggggcttggcgacagctttgcatagctcaaagtcgcggggtggattataatgtgacagaagtgttccagaggccaaatatctaatgaaattcttggacgaacaatacatcaattttctcgccccccgacttgataaattcagctgggaacgtgttggcgctgtcgccaaatttcgttgcccattatgtggggattctaagaagagtgccaacaagcgtcgtggtcacttcttttatgatcgtgatgacgatgtgtttcgtttcaagtgccataactgtaatgaaatgagcggttgggcgtttgaattctggttgaagaagtttgacgagcgtctgtataatgaatacaaccttgaaaaattcagaatcatgggggataccagttcccgcccgttacaaaaactcaaaccactacagcgcctgacccagacagcgcgtatcggttctcaggtcgccaaacgggatgaagagcaccttgggaatatgatacgccttgatcttcttgaccgtgatcatatcgcccgtcgttatgttgaaggcagggggatgccggagagcacgttatctttgttgtattacagcagaaattttcgtcaagatcttctgagtttcgaaacggatgttgagaagcaaaagaagatacccgaagatgagcggctggtaatcccattctggacccaagacgggcgcatgaagatagttcaggggcgcgcattcggcgataaccttcctgatggggtattacggtatgccactgtcaagcctaagaacgaagacactaaaatctacggggaagagcgcatcatttggaacaaaacaaagttggtagtagaaggtccaattgatagcttgtttctacctaattgtcttgcgactgccgatgccgacttattaagtgctaagggagacatctatatccccgataaccaatacaggaacaaacaagtttgtgacggtatacagaagatgatagacagcggagtcaaagtcgttctgtttccaccggaaataccgtggaaggatatcaatgatatggttcgccctgacaagggaaatatcccgatacgtgatctgcttcagatcatagccaagaacgtatatcagggactgtcggcgtccttacgattttctgacttgaggaagatatgatggatttgaaagaactgaatagtatggacgatgaagagattcttcgcgggtatatgcaagcccgtgaaggatatgtcctatctggcttagaatctaagagtttcatccatggttggaggaacggaatggtggacttcggcggttgtcctatcactgaagaacaaatggatttggctcgccagtatttgaaaaagaagccgacctaaaactttttaactcaaattatttcccttcaggcctcgttctatgaggccttttaaaatacttcaaaaataatttaaaaaattattgaaaaagactttactcttcaataatgatgcactattatagttcacatagggcggtacacaacacactgccgaatgaaaagtgaaacgacccaactacattatgaggaattacatcatggctactaccaaaactctgatcaccaacggcaccatctcttttgagctgaacaccgaagttgcaaaagttgagatgttccgcatcgctcaggcagctggcttcactggtggcaaaacttccttcatgaatctgctgaacggcaaagtgaaagcgaccaacggcttcaccctggtagagcaggttgtcgtagacaaagccgtagttgccaagactgctgacaaagttggcatgctgaaagacctgggccatgacatccacgttgtcgaagcgaataccgaaacttacggcaccatcgtagtcggtaaaggtcgtattcagctgaatccgctgaacaacggttccttctctgtgatggtcttccctaagaaaggctacgcaaacgaagacatcgtgaaagccgctggcggtgaagcgaaatcccagtatgtcaaaatgggtaaactgaacgccgacgcagtagaaactctggtgaacaaattggcttaaccgcaagggggaaacctccccctttcaaagttttggtaggagaatgagacatgagtagagcaattatccaagcctctcctggcgctctatgggttgcagaccgaattcccaacgtgcactttcctcagttccaagaggaactggaacaagcgatgttgaaaatatttgaaaaatatgggtatgattccgaagttaggacttccttcaaagaaatcatgcctgtcgtcgtagtcagttaatcgacactaaatcctcaagttcgactttcactttaaataccttcgcatacattgcggaggtattttcttatggccattctaaaacttggcaaccgaggttctgaagttaaagcgcttcaacaaagcctcaacaaaatcggtttctcccttacagccgatggcatatttggtaaggcaacagagaatgctgtcaaatccgttcaggcaggtgctggattggttattgatggtattgctgggccaaagaccttctatgctatccgcaacgctggagacgctcaccaggaacatctgaccgaagcggacttggttgacgcagcacgtgaacttggtgttgagctggccagtatgaaagctgtgaatcaggtagaatcccgtggcacgggttttaccaaaactggcaagatcaaaactctgtttgagcgccacatcatgtacaaaaaggtgacggccaaattcgggcaagcaagagccaatgctctgtaccaactctacccaacattggttaaccccaattctggtgggtatatcggcggagacgcggagttggaacgccttcagggtgcaatcgcccttgacgaggactgcgcttacgagagtgcttcctacggcctattccagatcatggggttcaactgccaaatctgtggctattcaaatgccaaagagatgttcactgatttcctgactggtgaacgcgctcaccttctggcatttgtcaagttcatcaaggcagatgccaatatgtggaaagccctgaagaacaagaattgggccgagtttgctcgtcggtacaatggtccggcatatgcgaaaaaccagtatgatactaaactggcggcagcatacaagagtttctgctaattctcaaggccggaaacggcctttttctttatctgatatacaggtatgctgggcgtgtcgcttaattaaaggtgtcgacaacccctgtagatataggaaggttcaacatgcaatctgcgtctaaagttgtatccatgaagccagcgaagacaaaatctgctcgtaagaaagacaccatccagaaagaagaagactggatgaaattttctaaaggtgatttcaaaattgctccgttcaatggcctctcagaaaatcagaacctcgcatatcaatccgcactcaatgaacatctcactatcgctatcggtccggcaggtacaggcaaatcctattgtggtgcatctgctgcagcaaaacatctgattgacaaaaccatcaacaagattatcatcacgcgttctccccttccaactggtaccacagcagggttccggcctggtgatacatatgaaaagttgatgccttatttgatgccgctcatacagacattcaaaaaggttctcaaaacagacacgggttccgacggtttcttcaactatctgtgggagaaacgcatcatagaaattcaagaccttgaaactgtaaaggggatgactttcgatgagtgtttcctgattatcgaagaagcgcaggaatgcgatatggagcagctgaaaaacttgttgactcgcgcttcagattcttcgtacatcttcgtgaatggagatatcaaacagtctaacaagcgactgcgtgatagtgcgttacagacgtatgtggattcctttaaagacttcaacaacaagctggaaacaggttccttgcaaattgacggtgtagaaatcggagatgaatatccagagtgggttcaaccattcagcatcatcgaattcgataagtctgatcgtaatggtcgcggcaacttcacccgcctgatgttagaaattaacgacctgtacaacatttaaataccacacgaacttccccgccgccttcaatccatggaggcggcgtataataacagcctgcataaacacaaaccgaggatcgctatgattaacatcataaagcgggacggatcttctgtcccctttgacattgaaaaactccataccgttattgaacgagcatgtgacggccttgaaggagtatcggtgtctgaggtggaagctgcatcgaagattcaattcaccgataacatgaaaaccgaacgcattcaggatatcattatccaagctgcggccaccttaatttctgttgataaacccaattatcaatatgttgctgcccgcctgaagtcgtatgacctgcgaaaggttgtctatggtcaatacaagccacctcatttgctggatatctttgcaaagaacaccaagctgggtgtttatgaccgcgaattcctcgaactttactctaaagaagaattcgaagaacttaacacagtcatcaaccacaaacgcgacaagaatttcacttgggcagcaatgggccagctgactcaaaaatatcttttgcgtgatcgttcttctgacagcaaagtgtattatgaaacccctcaagtcatgtacatggcaatcgctatggcattattctcagcgtgggataaagaaagcagactgacaatggtgaagaagttctatgaatacgccagtacaggcaaattcagtcttccaacacctattatgtccggcgtccgcaccccgacccgacagttcagctcttgtgtattgatcaaaactggtgatacattggactcaattaatgccactgcgaaaagcatcgtggattatgtttccaaacgtgctggcattggattcgatgttggggcaattcgtggcatcggtagccctatccgtaaaggggaaatggtccatacaggtctggttccattcatcaagtatttgactggtgccctgaagtcttgctctcaaggcggtatccgtggtggatcggcaacatgctatgttcccatctggcattatcaatttgatgatgtcgttgttctcaaaaacaaccgtggattggaagagaaccgtgaacgccgtatcgactatgggattcagattaaccgcgtcatgttcgagcgcctggtgaacaaacagcctctatacctgtttgatccaaaagacaaccgcgaaatgtatgaagcatttttcgcagacgtcaataaattccgcgaaatgtacgacaatatgatcaaggccgccgacgccggattatgtcgttccaaaaagctgcaagctgaagaagtgttccaaatgctactagaccagcgttctgacactggacggatctacatcgcattcgttgatcacatgaacgagtatagtccgttcaatttggacacgatttatagctctaatctgtgtttagaaatcgcgcttccgactcgtgagttccaacagtatgatgatgaagatggtcgcattgccctgtgtacgctcgcatcatttaacctgacggcatttgaagatccaacagaaatggaagatgttgctttcgttctggtttcagctttggatatgttgttggaatatcaggactatccagctcgccaagcccgtttggccgtagaagaatatcgtcctctgggtatcggtatcgtcaacgtcgcacatttcctggccaagaatttcacgggttatggatcgcctatcggattagaacttctagacaagtggatggcgcatcttcatttctacttggtcaaagcgtccaaccgtctggccatgcgtttcggatcttgtaagaagtcaactatccatgattccggatttgtaacagccgaccttcagcctctcccgctggacatcctacccaatggcaaaaagcctgttgggcaagcctatggtcttgactgggaaggtctgaagcagaacctatccgagtatgggatccgtaacgccacgttgttagccgtagcaccaactgaaagctcctctcaggtgctgaacgcaacgaacggtatagagccaccaaaaggtctcatcagcatcaaaggcagcaaggacggcgtctataaacagatcgttccggatgtagaaacccttggtcctctatatgacctgaaatggaatctggattgtattgaatacctgaagacggctgcggtcatacaacgttgggtagatcaatctatcagcaccaacacatggtatgacccagagaaatatccagaaggcaagatcccgcgctctcttatgatgcaggatattctgtctttctacatgtggggtggtaaaactctgtattacaacaccaacaaagactccaaggaagacgaggagctgaaacaagttgaagaaatttgtgacacctgtgttgtttgattaggtctataccacacaatcgtttaatatcaggggtgattcgtcaccccttattcatcaggaaagaaacatgaacgaacaaaagcaattttcagtatttgacccatcttcagataatacaggtttaccgttctttggcgaccctgtaagtatccagcgttatgacaaagtggcttggccttttgttcaaaaatggtatgaaaaaggcctcagccaattctggcgtccggaagaagtggatgtcaccaaagacaaagcggactttgccactctatccgctgcggaacaacacatctacttcagcaacctgaaacgccaaaccatgctggactctattcagggcgctgctccgttcgaagcgtttggcccatgggcatcaacaccagaagcgcaatttgctattctggagtggacgcgccaagaagccatccactcactatcctacacgcatatccttcgtaacacagtgaatgatcctggtatcgtttttgaccatgttctggacgttcaggagatcgtcgattgcgccggacagatcagcgtatactacgatgacatggtgcgttacagcgggatgcgtatggctggacgcgcgttcacccgtgaagacatcatgaacgccaagcgtgcattctggcgtgctctgtttgctgctaactccctcgaaggcgttcgtttctatgtttcattcgcgtgttcttgggctttcatgcaattcctcaacaaaatggaaggcaacgctaaaatcatccgccagattgctcgtgatgagcaagatcacctcatcctgacccagactttcctgaaccgtctgccactgatggaccctgattttgccattattagagaagaactgcgcggcgaaatgacccagatgtatgtcgacgttgtcaaccaagaaaaagaatgggcgaattatctgttcaaagacggctcaatgctgggactgaatgcaaatatccttcatcaaatggtggactggttggcaacacaccgtatgggagctattggccacccatatccaggacaagcccgtaaagacaatccggtgccatggataaatgagtggctagataataaaacaatgcaatatgcattacaggaagcggaagcccctgattatctgactggtgtccttactggatcagtctccgacggtctgaaatttgcctaaaggtgagaaatgattacgatttattccaaacaaggttgtgcgcaatgtttacaagctgaaaacatctgtcgcattcgcggtattgaacaccaaattctgaagctagacaaagattacaagctggaagaattacagaaaatcactggtaaacagcgcatgtctatgcccgtgattgtcctggctgatcaaaccgtgaccgatgttactggactcgccgcaagtttgaaacgctgatctttcaaagcccctcaatgaggggctttacttttgaaaccttcccacctatacttcccagtaattcattggccaacgccacaaattgaatagcaggaactatatcatgaccaactctgaaatatatcatgcttctatgcattatcacgacctacgcgcccgtccaggacacgccaaacaaaaaggattctgggtggcagaactcagattacgccgtgctgtcgttaaccaccaaataatcatgaatcggaaaaacgacaaatcaccgttgttggcagattcttatcttgaactccgcacagcctatgaacaagccaaacagcacgtaataggagcataacatgtccaacaaattactcaccaacccccgccacaactttggttatatccaattaccagaagtgatagacacgttgtttgctgaagacaaggaagaagtttgttgggcaccagcgacccttattgaacgcgaaattttcaaatctgaagtagacgtgatgttccgtatcggttcatccggagacctccattatttggggaaagtgaccgagttaaagacctgccatgctcaaagatttttgcgcattgacaccactcaatgtgaagacttttcattcagcattctgcgtgaatatgttcaggtcaaccgtcctgtgtatcaacaccgtccttatttgacgtcgatgcatatagacaattatatttcagatatccacttgcgcgcctcaaccatacatgaagaggattctggtgtcgcggttgatacggtactcaacatgattgtgattacgttggtgccatgatatgtataaattcccgatattagagacggtgtattccgtcaataacaaagaagaacctttatttgaaaatttagataccctgttaaaggaaagcgcacatgttcctcttgtcctcaaagggaaagaaatagggtacagccatgactgtcaaataatgcgttctgaaggcagtgatatccttcgcattcgtttaacatttgtgttgacggggagactagatggtgtggtggttaacctaaagccaaccaccatagtccatagaataggtgggaaattactgtaccgtttactgacgcattttgaaataattaccaaccgctccaataccaacgcaccttaatcagtttgtgcgttttaaccatatggtgtctgttatcggctctgggttgttgaccacatagaacgtgatggttacgcccagagaatgataatcgtcagacaatgatacatcaacagattgcaattcagctcttggctcaaaaagagcaatggcatcttcaactttgttcttcacgtcgacctgtatcgtgggatttgtgttttctcccagcatggtatacaaccccgccccaatactcggatatgtcggccaatcacctaccgtcgacatcactatattacgcacagattgtagtacagcataaatgcctgttttcttagtgacatctttggtgaccggatgcatgccaaacttcaggtcaatgtccttgtactctttcatgttaaacccctttggatattgcctctccagcacgagagggcattatctgccctcaccagtgtaatcatgcgcctttaccaccaaaattgccgccgttatcctctaactccggttttatatcaattggacctgggcctgtagttgttacaggagttgcccccgcacgtgaggcggcgcgaccagcgccttcagcatatttgatgttgccttcaatagtctgtgcaaccgataatttatcacaaattatttcagttgccagtatcttaggaaccttcaaagtgttagacacttcaaacatttcacagatgagacgcaacacgttctgtgcctgtatctcagccaattgggaaaatttcatcaacgctgttccagcgaccacattggaatagttagaagagtgtaagtgataaacttctcctgtcttgcgctgtacttcagtcccaccaattgtcaggttatgatcacgccccacataataacgtttgtcgaacatcgtcaggtcataatgatctttgactgatttattaacaacatcaccgtcaggaagcatttgcttgtaagacccagaagagtgcatccaatgcaaacgttccccgcctggagtgtcatcaacctccataatatgcccagcgcgagaagccataacattgttgtatggataacgagaccccccagccgaaggaaccgatacctctcctgtgttgtcaatatcctgaacacgaccagaaggaggcggtgtttcttggactttcggggtacgatctggaataggttcagaccatttaccgttttgttgtgccggagctgctgaagctgtagttgctggacctggccaacgataacccaacacggatgagcggctaaaacggctcaccttcacagaatcggattggtttcccccgatacaccaaacgtagttggcgtcaaatttctgaacgaatgcaacgtgaccgaatgtcgggttgttcccgcgccggaacactacaacagcgccgtaacgtggttctgacagaggagacccccattgtagatatgaacgagccaaagcagaacgtgttgatgtatatccggcctgtatcaggacccatccaacaaacgacgcacaccaactaacttcatcttctgacgcccccagggaagttgtcttatgatattccaatatccttgggttgttattgaacttaccagaatattctttgacgcccaattccccacgagccacggtcatccacttctcaggatcatatccgtctactggtggttgtggatccggttgtggttcatcctcaatcttaacaggaacattctcgacggcattatacttctgtctttctatagattgaacgacctgacccagcgccaatgggtttgtatctgacccatctgttggtgttgctgctggccatacccatgcaatacggatgttctgatatgcttcatccaaagcaaaccccatgacgtcagagccgacagtgatacccgtcggagaccagcctaaacctgccgaggatgcgttagatgctggcataagcattttaccccaaggcaataattctgtaggcaggagagtggtatcctccgtgtgtaccccatagatgcgtactgcgacgcgcccgttttgatcgggatcattcacgtcttcaacgcgcccgtaaaaccaacgcaagttgtctaacatattgaaacccattagtcaattagaatcatgtgatatttatggcggggatttgaaatctgtcgaataatatattataggaagctctgcttccgttcaacctttattcttttcgttcctactcgctgaagctcgtaacaccgccttgacaggcagtcccattccaatgccatggaggcataaaggcgaggaaaatactgtacacctcagaacatgcgtagaaaaggattatttttgcttcaaaacaaattcaaaccccaattacacaacacttacttcagcaacaacggtgttatagcaatttaaggttaaacgttcctgtcttaacctataaaacaggtaagcaaagaaaaccccacattacgtggggttggaagatttcttagacaatctaattttcagtgtctcggtaagcaaatcaagatgccacggcatcgattcactgatttcaaatatgctatagccgtgtgcctttaattcgtcacacatcacaaaataggagagtaaatcaatttcaaaaattaaatgaaaatctcggtgacactgttaaatttgacttcgtgttctttcccacacgaagggcatttaatttttgtcgcgtaacgaatacgcggaatctttttaaagaaatcattcgaaatgtcctgaacaatctcagattcaatattgtccccgacccatttgacaaattcatccttaatgcgttggcgttctttagcaacttctggatcgataccaggttcagccggattttccaccttccaaacctgaccatcgtcgtcatacagacaatcgataaaggtggcaatcatttgttcaacagaggatgcttcgttgagcactgaggcatccgagaaggacggctggcgcatctttatatgataaccacctggtagatcaaacgtctccctgaagccttctggggacacgcattttacttgattgagcgggatcggtaacacaagctcttgaccacaatctttaaggccagaaactggttcttcaccaccttcattggtttcggcttgaactttgttattacatttgtaacgtatcttcatgacctcgccgatagatatacagcgcatctttaaaaatacttcttcagtaacaccgattggcagtttactaaagggaacgccagcatcaacacaactgtcaaatagttgctccaatgttgccacgcgctcactcataggagtgttcggatcagcaacctgaagtaacatggtctgttgccctgctgtaaaggcgcggtatttgatcacagtcggccagaaatcacttttatgtgttctctcagttttgggcaatgatggtaaattcataatgtatctccacggtatattgtaattgtatttataacgaggactgaaaatgcacgcgttctgtttcagtaaagtgggggagcaactcgcaattaattatccggatgcaaacacggacttttttgtattactcacagattttgcaaataaattggggataaagaatgtcgccatgaccgtgtccgatatgaaattcgggtggctgggagttaaagatttattgaattatggattcaatgtgattatcgttgatccaacgtacagcccaattgaagaggatgatgttattccggtttgggtgcaacgagatattaacaccctggacaaacactatccggattgtatcatcatcggtgaattaataccgtattttcataaacgtcgtcgccttgaaacaattaaaagttattttcaagataatgattgtggatataaaatggatgtccttacgagcggttgggttgtgaagaatactacaggcaatgccgaccgaattacttcccgtctttatgaatatgttaaagaacgggattatgaactggagaacaataatgccaaacgtagaacgcgctgatattcagcaggtacaaaaagttatccttcaacgcctccgcgctgttatcacaaatgaaaagggagaaattgcgccagggtttgaaggcgcattcgacggtttcgttgccgatgatcaggggaaccccgttgtacaaacgatcgctggtatgattatgctgaatagtgaattcatgtcggaagggaaaattcattattcgccgaaccttagtgttgatggtgagtctttggcttccgaagtggttgatttaacggttgccgttggtgagttgggttatgttttgatggtttgcaccgcgcattatgtggacgccaacggccatgtgtcatatggtgatgaagcgcgggggatcaaacgtcatgtggacacttctgcattactgcaacagatacgtcagatgcaagaagaaatggactccaaacctcgcctgattttaccggaaagcaaaatcgtcaccagataatccggtctaaatacccataacacaatgaaacttcgttatgggtatttttatgaaattttgcggaatcgactattcttacggctgtccagccatgtgcttctgggacgacaaagatcctctggattttgatcatcttcatttctatgcacatcataccgttgagaagcactgtcgtcaagtgcgacacaatattcttattttacgtcaacccaaatatgaatcccccgaagaacggttttataacatttccaaatgggctgaagccgtacttcttacagagaagccggatttcatcaccctagaaggctatgctatggggaattcaaaaaactccaacaacatatgccaaaccgccgagaacacatctctattgaaacaagcgatgcgccgtaacaatatggaatttcagattgttaccccgtcttttgttaagaaacatttttgtggtaaaggaaatgccgacaaattggtcatgatagatcactttgagaagctgttcaacgttaagatgcgcggtataatggacatgttggatgtgaaggatccaaagcctatcgatgatcttgtagactcattcgcaaacatggtgtctggaccttacttcatagaaaattatcccgatttcaatagaggtgtaagaaatgattaattactggctgttggcagacatactgcttttcgccttgctgttgataacggcatttatttgggttaagggatttctgacattcctacactcgttgagtgccatgatatctttctatgctgtgtccccccatagtgacgtcaatgtacgagcagcaaatcaatctgacatgttggctgaatacgtcatgatgcgtgataaagcaactaggatcgtcctgttttctacactggcgggggcggttattatattcatacaacacgtcttggaggcaactcatgccgttttatgattatgcctgtacaggttgtggtaattctttcagtaccagaaggtcttgtgctgaacgccatacgcctgaattagatccatgcgctgaatgtggcggtgaaatcaaaatggtcattggagcaccaaagatcgtatctggtgtccgtggcccacaatccgcacctgatggcttcaaagacgtattgcgtcatattaagaagcagtctgggaaggggaatacaatcgatgtctgaccaacaaatagcacaagtcctcacacctgattcattctcagaattggtgttgatgcgagcgagccaacgcaaggagtctattcttgaaacgatggccagtgtttgtgaagagtacgacatcgaagaggcgaaagttaagaaactgattacaccacctctcctgtcaaggctaacagctgaatgttctgatgctcggttgttgaagggtgaattgaaatctaaaaaactcatttaaggttgtaacatgagcaaagagcaaatttataaattgttgaaggcgcaagagtatctgccaggttctattcgctggcgtcatggtagcctcaacgaacatgctgatgatatggatcgtgtacgttacacgacgccggaaggtaaatcctatgtcatcgaataccatacattcttggaaggacacaaaaccttctcagatgtgtacgacattattgaaatagacccagcaaaacaaatgattgcgggttaactcaatagcaaaacgcagtataattacaccgtcggaataattgacaaatacatttacagaatagcccgaaggggcggattatagaggaatattaaaatgggtaatttatttgatcgtcttaaacaatctcgtggccaacaagccgaagctatgcaacagcgactcgctcagcaaggccagcgtgttggtggcggtcgtgacccgcgcatctggaagtggacctggaatgacaagggtacttctgaaaacatcattcgtttcctgccgatccctttggtggacatgaaggctcaagaagaaggcaccatccctgaagatgctgtgttaactccgtgtgccatgatcatgaaacacgcattccagggcgctggtggttggtatatcgaaaactctccacagactttcggtaacgatgatcctgttcgtgaccatgaccgtcctctgtgggcacaacagaaagaaacgaatgatgaaaagctgaaaacagttctcaaaaaacgtctgccggacaccaaatactacgccaacatcctggtgatcaaagatggcaacaatccggaaaacaacggcaaggtcttcttgcttgagttcggtaatgccgtcaagaaaatcctggattcggctcagaatcctaagttctcaactgaccctaaattcgacccgttcgatatgtgggaaggcgcgaacctgattctgaacctctttggcgaagagaaagagttcggcaactggaaaggtctggtggccaacttcaccaatgtgaagtgggatactccggcacctttggggacagacgaatacattgaagagatctgggaaaaagagcacagcttgtttgagttctttaatccggccaacttcaaatcgtacgaagatctagaaaaacgtttgcgcaaagttctggccatccctgataatcagcctctggttgaaggtggtgcttctactatggcgcatgcgcctacccagtctcaggaaccccagcgcccaacagcgcaggaaagtctgaaccaacaacaatctcagccttctaatgcacagcaatctgttccagccaataatggtggtgccgatgcgaaacagacggcttccattgatgagttcgagcagttcctgaagcaagactaatttcgctaaagccccttcgggggctttttagtttttgacaacatcctgcaacaatccgagtaaactactcttctcagacgccgtcaggttatcattgcctaatatatccctctggatgccgatagagatcctttcgaaatcagccaggctgataccagccagattgtttacaagctgatcgagttgcttataagccagcagggcttcacctgtgaagtttccttctttgatctttttatacagctgtccggcttttgacccagcggtttcccagtctccggaagcaagagcatcagtaatacccaaaggaagaaaatcgctggcgaggctgcgcgtttcatattcagtctccgataatactttgttataagaaaaggaaatgttgtactgattaaattgatcggcggcgcttttatccagatctatagaactgaagttgatggggtgagcctcagtcacataaacgcgatgaacaacctgatcttctgtatccatttgctctatacaaatatcggtcacgaaatcttcataataacccatcttggttgtgtatgggtcgaatatcagattcttccatttgtccatgaccgacttttcataataatcgttggcgaggaggaacgacaactccagatcaatgttcgtcttgttgttcggcattttaatgtggttgccgttgttggtcatgggagtagtgtcaatccctgtaccaggtaaagatgcgaccatacacatcatttgcagggaacgagatgtttggtttgtccctccaaagaatgcgtttacaatacgggcgctttgtttaaataaatcgccgaatgaagatgaaggatacgcgttcccatcatttgctagtgttgcattggaatcaaatatcccaggcggcaatggaattgtaacacgaaatctgttcttgcgggaaataccccgttgaagcagttgcgttagaaaattgcgatagtcttccacgatgaggactccatttaaataatatatctgttgatatttataaggagttgacaaatggctaagaacgctatgggtgaagaagacccgttgttgcttcccgccgagatggacgccccagaactggtcaagcgttacatccgcaaatatcgtcaacattttgggccggaagcgaaacgtaatatccgtcgttctcatgtgtggtttatggagcgcgtatcgaaagatgctaacttatctccgaatcacatgatgaaagcattcgctgaaaataaacgtcctgttcagggtgtgcgttatattgttggtcgcatgtattatttcaaatatgatgcgctgactaaagatgaactcccgtattgggatatgtatcctctggtgttcttcttcaattttgtgaagggggatggggtgaaattcggtgaacgtggtgtgacctatctttatggtctcaaccttcattatttgccaccaaagctcagattattagtgtttgaagatcttatcaagttgaggaacgaaagagcgtaccgttctaaaactcgtttgagacttacctgggatgccctgaaacggttcgccaatcaccctctgtacaaccattgtgtgaaactgtaccgcgctgatcaatttcgttcacagctttatgagattgagcctcaatactgggaagttgttttgtttatgcgcactgctcgattccagaaacagagccagatgtctgtttggaaggacgcacgtcgtaaacacaaaaatggttagttctaaaagccgttctacgtgataattcgcgaacggctttctataaacatgaggttatgatatgtcacagctacatcgtatttttggtattgaattcagttctaattgcgttcgctatgatcgcacccataaagattcgggtggtaaaaccggatatgaactcggtagccagttgggatattccaagggacaattgtttaatcagcacaggctgaatgaaacggatcttcttaaggttatgggtattgaacttgattccgaagccagcttggatctgggcgacgaaattcgttttggcaaaccagaagcattggaattcattgaagacttcgtttctggttatacccaagccgctacgaaccgccgcgctgctaatgatgcagtgggtaaattctactttgcggcgaaatatcttgccgacaaacgtatacagatggaagcggaattcagtaaagaaaatctgaaagcggttttcggcgaagaaggcgatttccgtttaatgccagaatctggcactgattcagctcgtgatttctttgaaaaaccatttgtggtcaaaggctgtaaaattcttaccgacgaagaccacgctttcgcaatccatactgaagtcgtttacaatgaagagaattgccttgttatttccagattgttcaacagtctgtcatatacagcgtattctttgattgaacgttgtatggaagaagagcatcgtcaggtggtggtgaacaaaattcgcttacacgttgagcagaattttaacgggtttggttgtgaatccggcgaaatccctaatttctatctgtagcacaagcagggggctgtatgaagaaatatgttcgcctgaacacggtactggaatctgtaatcgaatatttcataatgcagcacctcagagcagaaaatgatcatgtgagcgatgctttgacttatgaccaccggatagtggttcattccgtcgaaatagtggatgacgaattgttgattgccaatgttgagcatgctctctgggatgaagtgttagatcagtatgttcaaccgaagttcacacagatcgagttgtctgccatgtggccacctgaaacagttttcaatattgatatacaaagttaatattcactccagtccgcgctgatacagcgttccgttgtctaacacagaagtgttaaccaacttttcaataaaaagtggtttacatcttctaaaaaggcagtatattaatgttccagacgcacagttgaacattaaactgccaaagaaggttactacacaaaagggtttttattatgtcctatattcttcatatcgagtccggcctgaaatttgaaattgatggtaaaaccgctgctgatcttcaagcagaaatcaaaggtgcagaccttatcattggcaatgtaacattacgccgcatgatggaaggcgtccttcagtcagccaatggctttgaactcatcgaaggtctgagccaggaagagaaagaagaaactctcgaagctcttgaccgcgctgaagccgaaaaagttaaaccgaccgaagatgcgggggttaaacagggcgacggtgaatcaaatccggtgattgacgccgaagaaacggttgggcaacctgtcggtgatcaaaattcccagaccgtcgaagacggttctcttgaagtcaacaaagacggttccatttccctggtagttgaaggcgaagaagttcttgacgaagaagcgcaagcccgtgctgaagaagttcgccgtcgtatgctgggcacaaccgttgctgaagcaatcgccgacgttgaaaatccgaaggatgatcaggctttagcgaacgcgaaggcggttctggccaagaaatcttctgaaatgcgccctgcatcttctaccgaagaacgcaagcgtcgtcataacaagcgtgaagaaatggttgaagcggctcgcgcatccaattatggtcctatcctggctgcggttgaatcgggggttgtccctggggtttatttgagctatgtgaatccggatatgcgctggttccagttccctgttaccgaactggcagatgaagccaatccacacgcccgcaccaatacttatgttgatctggcgccgatcgtttccggcggctggggtttcagtctgtatgtgaatggtaaatcattcaccaaacgccagaaaatcaaagaaactgatgccgaatctctggtgaaagcaatcaatgaatggttgcctcaggcgttggccgaagcgaaagctgctgcgtaatttaattcagaagcgttcaatagataaaggggctgctataatagcagctccttttgtttattggagcatgtcatgtcttattcgctcaaagggctgttgaagcgccctgtgcatttgtttgtcaaaccacctgctgtagaaggggaatattcggcacgaggagagttgtattacattaaaggctccaatggtagtggtaagtctactgtgccttcttatttggcggagaatgatcctcaggcgtatgtcgtaacccataacagtaagatcatgctcacggtttgtccatcttacaatattgtctgtgtcggtaaatatgacaagtctaagtccaaaggggttgattctctgaaggatactgagcagatgttgttcgcgttgtctattgctgaccaaccagaatacctgaaatatgatgtgattttcgaaggcatcattccgtctacattattgagttcttggattccccgcctgacacgcccaccacgtgaattggtcgtcctctttatggatacccctcttgaaacctgtattgcccgtgtcaaatcgcgtaacggtggcgcagacttcaatgaaagtctggtggtcgagaagtgggagagagttcatgatcaccgtcaacggcataaaggcttgttccctaccgttcctgcaggtatgatgaagtccgatggcctgactgtagagcaggctgtttttgcgtttctcaatcgtgattttgggagtatttattgatggaaatcaaagcaattaacaacaacgacatgctgaaacaggctgtcttggctatccgtgagcacgggattgaatctgaccctggtaacgcggagatcaacacagacggcactcgttttattgatggcgtgacgatcaccgtctctgacatccgcgaccgttggctgtctgttgaaggacgcaactcatcggctattgctgccatcggcgagaccttctgggttctgtctgggcgcgatgatgtgcgcttcttgtcccgtgtcctccctcgcgccgccaacttctctgataatggccatacatggcgagccgcttatggtccgcgtctgtacgcccatggccagctggatagcgttatcaaccgcctacgcaagaacccaaatactcgtcaagcgtacctcaccatctatgatccggctctggattcagatgctggtctggcgaagttcagcgagagcggcgaagcaaagactaaggacatggtgtgtaacctggctctgttgttcgccatcgttgaaggtcgcctgaacctgactgtcatcaatcgctcacaggatgttctgtggggtatgagttcaatcaacttcattgagttctctattcttcaggaagtgattgctcaggtgttagatgttgatgtcggtcagtacaagctattctctaacaatcttcattactacaacaatgaagtcagccagaagcagcttggcaagatcaccaaagacacaaaagtcactggtggtttcatcaactccatgatctacttcacgaacgtgaccaaccagaaccatatccgcaatctgttcactggtgtgcttcatcactgtgatatcggtagcccgtgggaaactgttgttgctcacctgaaggagtacggcgcggatcgcggcctgatcgtgcagatggcgtactgcctgtactgcaaactgaacgacaagctcatcaacatgaacctgattcaggatcatggtttgaacatcgcgctcgcccattctccggttgaccgcaagatcgttgatcccaagttcttggaaggtgcagtatgatgtatccgaaccttgaattctttacggggcgcaagcgctccggtaaggacttctgtttggagtccctcatcagctatcaccatttacagggtgatatggacatccagcgcttatctttctcggacgagttgcgtcgggtggctcattacatctattcttggttgccagaggaagtagaggacgatatcaaggacttgccatatgttcacccagataaccccaagggattaaccccaaggcagatctggcttcatctcggcagcgatacggggcttcgttatgtccaaccggatctgtttttgtctttctttaagcgttaccaactcccattagttgaacagaatcctaacattcattatattgtgagtgatctacggacgcctcaagaatatgaatgggcgctgagcactaaatgtcccatcacccgcatttcaaaggcagatcgcggcggtatcatagaggacgacatagaggctttcattgatgagatgaaagttgattatgaatttgttaacccatttgaaggttggcaaccattcgtcaaattttatagggaacggaaatgatcacagcagagcatatcaaaagtctgctcgaacttcaaaaggccaccaatgtggcctattttggggaagagtggcgtaaacattggactcatgtggcggtggtgaactccatctatcgtgagtgggcagagtttcttgacgaaacaacccaggactggaaaatctatggcggtgggatacaatatgaccgagaagcagctgtctacgaattggtggacgtggtacattttatgttgtcttccattttatctggatttggttctgctttcccagttgaaatgcagattgaccttttcttcaaggacggacgagtgatagacaaatcggccaatctggaatcggtcacagaatgttttacgtcatttatggaacaaccagatgttatccgttttgtgcggtttttatcaaaggcttgtggttatttggatctggacaccgaaacctacatgctggcccacaagcgcaagaacgatcgcaatcgtctacgtgctgctggcggtgcgaactatgataagtccgcggaaacccctctgactctggagttctaatgtttacagtcgtcgtatcatattatgcggaacatgccccgtacatggcagagaacttcgctgagtttattaaacctggcagacatgtcgttatcgcgaccaaattcaacaaagaaccagatggtgcggctactgtaacgcaacatattacaacaccagagccggaaacactttttaaacgcattgaagattttgcttttggcggggaagaaattccagagtcattacagcatatctattttgaagatgcgatatattatgttcatgctattccaaaggatgttacaactgaacaaatgatgtatatcctgaatatcgcattgaatgtttcttgtgtcaacagggcacagcacaatcgtctgcgtaatatgtatttaacagaacgcgggggttccgaaattaacgatgaaaaactcccgtcaataatggcgattcacgaagcaatcaatatattgtccaagaaagtcttgggcaccgatttattaattacgatcaacaagcaagaggaaacgaaatggccgattcattgatggctcgcatgctcaagacagcaaagaaactggacccgaatgccgaagtgctgtcaaagaccgatgcgctgaagcctgacattatttgcagtacgggtattcctattctgaatttggcctggtccggtcgtattgatggtggtctgatatcaggcatcaaacagttggtgggggattctcgtacattcaaaaccatgtttgggctggtggatgttaaggcttacatggataagtttcctgatgcaatttgtatcttcgcggattccgaaggcggtgcgaatgaaaactactggacatctatgggtattgacatggaccgtgtcttgtatctgccgattgaaaacgttgaaaagacgaagatcaagctgacacagcttctgaatgatgcacagaaaggtgacaagatcatcgtattcatcgactcaatcagccagttgccgtctaccaaagaagttgatgatgccatagcgggcaaagacacacaagatatgacccgtgctcgcgctctcaatagtttttggcgtgttatcactccgttggtcactgaaaagaaattcgttttggtatggatcaactcgtactatgatgaaatcgggaaccaatatgccgagccgaacatcaaaggcggtaaacaggggttcctgtcttctaaccaattgtggttcatcacgcgttctcaagtaaaagaagacaaagaccttttgggatggcagtttacagtcaacattatgaaaggccgctttgtccgtgaaaaagccaagttccctgtcactgtgttgtatgaaggtggtattgaccgttggtccggtatgttagaaattgcgcgcatgctggggtatgtggatttggtgagtggttcttggtatcaacgcactgccaaaggcgggtttgatccagaaaaagaaaagaaatatcgtaaggcagagctgggagatgacttctggtatccgctgctggaaaacccagactttgttgacgatgtgaacaacatgttcggtatctctcagagttcggttatgcctgcagatatgctggagcgcctcgataatgtcatcaaaacaaccgagtaatattgcagggggaggagactcccccgttaattacaaaatcatcgaccctggttctgatcagttagcaataatcgaaataacggaaggcaaattccgtggcgttcaatttcgtatagggaaagtgggtgtccatttagacaatggagagcctcggttatcctttacaacagatatattgaagaaaccatggcgtttgttatttgttaatttgaaagaaaatgacttgttcactgtggtgtctggggatatcttagttgatctgatacagcaaaatgctcaggattacaacaaaattttggtggggtagttgccaatgttactcgaatctgtcgtgctttcccaattaatctataacgaagaatatcaaagaaagatccagccgtatttgaaagccgattatttcgataacgaaggcgagaaaattatattcggtctcattgaccattacacttgcgaatataatgctcgtccttcggttgaagcgttgtctattatgctggaaaagacttcgctcaacgaacatgtatttgaacaagctatttctgctcttgagaatatcaatgacaacacattccatcaggaatggcttgtaaaagaaacagaaagttgggcgcggcagaaagctgttcataatgcgatcaaacacgccgtcaacatctatggtgatgagaaacgtaaagatgagatgaacacgattccaactctcctacaagaggcgttggcgataagttttgattcttatcttggccatatctattgggaaatggctgaacaacaatacgaccacatgaactctaatgaagcgaagattcctttcgctgtagagatattcaacaaagcgactcgtggtggtgttggtaagaaaacgctgaacatcgtgacgggtgcaattaatgcgggtaagacaacaactctgattgatttggctgctggttactccgagcaagggttgaacgtattcgtattcaccctagaagtggctgagaacgtctggcgtcaccgccttgatgcccgtatgatgcgcagggacttcgagtccttagagaagctctcacgacatgaatatgtcgctacgatacaaaagttgcgaactcgtcaagacggttccatgaagggtgatattgttatcaaggaatatccttcgggcgcggggcatacaggtttgtatcgtcgtgatattcttgattatgcgactgcaacaggaattacacccgatgtcatcattattgactacttgggggaatctgcatcttctcgccttcctgcccatttaatgcagaacaccaacgtgtattatacgtcggtggcgcgagaattccgtgcgcttggatttgaatttgattgccctgtatggacaggtatgcagtttaaccgtgaaaaacaatcggcgactgatggtgatattagcgacctggcagatgctatcggtattccgaaggttgcagacttcatcatggcgttctatgcccctgatgaactggcggctgttaagaaagcccgagcgtctatcttgaaaaaccgttatgccaataaacagaaactcaaatcgttcttgttcggtatggatcaagataaacagatattgtttgacttggattggaatgaagtcaagcgagacctgacagaagaagaagcccgttatgttgagaatgttcatatcaaacatgacttaaacaaaacaggcgacgtaaatgatgtcaagaaagccgagaccgtgaacaattggaatttcggttaacttcaataatggtgatcatagggtataatctctatgatctgaatattaacccaggagcacatcatgtcggatgttgcatttatagctgacctgattcgggcggcacaacaggtgtgccagtctggagaaaccagtctcaaactcactacagagcagactctagaattttataagaatcgtcggcattggggtaagaatgtcaagatacattgcgaaccaggtgaattggttcagcttgatcttccgtcttttccattgccagtacagaccaacactcaggaatattacaatgttgaaggcgttgatattattcctaagtttggttttcacagtatcgtttacttcttcctgcgccgtttgggaaccagtatcggtccactcggttctcgtcattaatgaggatatattatgtcagttgaacaaattggtttttatcaactcccttctgatccaaaactgcgtcaaaagatgatgcagactttagaaaattgtcgcgccgcccagatccgtatcaagtctgaacagacatttgttaccgaagccctggctgaattggcaaaagaaacaggcatcaaagccgttgatctgcgcaaagtcgttaccgatcgcgctggtggaacttataccaagaccatcgagaccagcgccaaataccaagatctttatgagtcgctgtatcctaacgccgcgcctgatcgcactgctgaataatatcggtaaaagcccaacatttgttgggctttacttcaataaaactttacttcaataaaacttgcggctatcatttaacctacattgaaaagtttcactacatcatgagaggcaaaaatgaaagccaagaccttcagcatcccaattgataatattggtcgtgtcaaggaacgtcttgccaaacttgaaagaacggcaaaccgcctgaaactcgagtttcctcttgttgagttcagtgaaccatacaaaacacaacaccgcgattctatcactggcgaaaaattttatcgttggtggcaagattgtactctaactggtgaaggcatcgaccgccccgtttcttatggtgggtggagtatcattggacaattcaaccaccagtatccaaaagtcattctgaataaactgtcagatgacatccacccaaatttcgttcaaagattcgaagctgaaaacgtatcttggtgcgagcattgcaacaaatctgtgcgtcgtcataacacatatgttgttcgtaacgaacaatctggcgcacagatgctcgtgggtagcagctgcatgcatcattatgtcccgcatcagaaatcccttgatgccgtgatgtcttattatatgtcgatacatgaaatgttcttacctgatgaagatgaccctgaaggcatctatcgagtgaacgaacctgattacgttgacactgaaggatatttgtgtaaatgctttcaggttttgctgtcgggtatgagtatgaagagcgatgattttggtcgagttcttggtcatatttctagcgggactcgtccggagaaaggttctgacattgagatattctataacaaggctgttaaagcccgtgaagatgctcaatcagagatgtaccatatgatgcttttcatcgccgcgttgtctgagaacaatgatttcaacgtccggctgaaacgtatgtgcgaacctggttatcaccttgttaaagactccacaacagttcgttggggagcagcaaagtattatgattatattcacaccccgcgccaaacgcgcactgtatcaaattgggttggtgaagttggtgaaatgttagaagtacaggtcaaatttgaagcaaggattttcctgtattcgtctgattatggtgacacgtatctgtatactttcaaaaccaaggaaggcaataccattacatggaagacttcctatatggaaaccgagtttttagaaggcgatatgatcattcgaggtcgtgtcaaagaattgaccgaattcaaaggtattaaacaaactcagattacaagggcaaaactgaggaaattatgattcttttacaatttgttttgaaagcatttgcattcggtcttatcgcatgcggagtgatcacagctatgctgttaatcatagccctgatcatcccatttaagaggaaacgttaatgaaacgggaaatcttggatgtttactaccctggatcttgttgtccaggccatgacaagtacccagcggacacatacaaaaatcgccgctctaaatcaaaacgttcagacggtattaaaaaggagcatcgacatgcccgccgtgtcaagcgccaaaatctgaaatctgatttgctcttcattaccaaggaggatttctaatggaatgttcatgcggagggcaaggcacaagtgcatacttgccatttaacaccgtgaaggaagccgaagaagccggatacgccgtggataaagctccctgtgtaatcgccaccaaggattgtccgtgctgtaagaggcactctcaggacgtgcattactcaccaaacgcacgttcggataaagtcaatcttctttcactgatgagtaaatgacatgcgcaaatattttaactggttgttggggaaacccctaagcgtcgaaattcgttgtcttattgatgctatcgacgctgatgacgttgaatggaatggtgaacgctttgtcggtatcgatgctgtcatctataacggcgtaagggtatgtgcatatacagtcaatagcaacgcagggtatagagtcgatatcggtttttgttacgattgtctgccataccatgaggccaaggccgtgtataaagcgctcacccgacaccacaggcgtcggattaatcgtaaagcaattcaggtcgatacagatgaccgtgaaaaagtccgcaaggctcttggctatcacaacaaataattcaataaacgtttacccatataatacatgggaattaaacagaaggaattaaacaatgtccacactcgtagaaacattattcccaggttgtacgcttgggcgcggcggctctgaggccaatggattagcgattgatgttgagcctaaaaactgcaccatcatcgacaagtttcacgacaatcattatgctctgaatgatcgtcgcgatcattatttggtttctgtgggagaatctttggttcaccttcgaatctgttctgctttaccagggtatgggtatgacgcatttgcttatatccatgctgtttccaacggcgagatggggaaagaatccctgattgaaaaattcaagagttggaaggaatccttcacctctgtcacaccacctaagaaataattgaggaatatattaatgtcactgaaaacctttttaaaccgcctgttcagcactgtcgaggccaatttaactgcaggtttggataaactcacagatactgagaagcgtttgaatctggctgcacggaaactatctgatgaaatccggcgtttagaagaagcccgtgtaaccaatgtccgccatggcgttaagatgaagaagaccgctgatgaaaatctggctgaagccatcaagcgagaaaccattttgaaagatgcgatcgctcgcggagtaactcctacccgcgccgaagccttgatcatactccaccgtcgtcgtattggtgaagctcttctggataaagtcaaagagatcgaatctggcagtgtgaaattgaattcggcaattgttgaattaggtgacaaactggacgctgttaagtctaatctcgaattggtccgaattcagaaagaatcaaatgatcttgggttaactttaccggaagacatcgattacgcggctggaatgattaatgtagatgttgatgctatcctgcgggaagctgaaatcgctgattccaattattctaccaatagcccttcagcaacagaagcggacaattatctggcaagtttatcaaaataatatagaaggggcatatgccccttcatttgggggatttatggtcattattgactcaacagatattttgattgctgttcttagtatggttggggtctgtattctcgttatgggaatcaaaaaatctttgggaaaacgggttgttgaggccgtctcctcagatttacaaccagaaaataccacgaaaaatattaaagacataaaacttattctgagtagtcgtgtgtccaaaagtttggttcctagcatagatgatttcaacttagaccacaaagacccagacactactcctgaagaatattttcaatatttcatgaaaggtttgggttggccgttcatagaatcgtgtgtatcaaaatcaaattgtttctatttgaaaatagcatgtaaagatggatatcaggctgctgtatggttttggaattacaacgacacttacgataaacaatttttgatatatcttcacaggttgtttaaagagtatatgaacaatggaaatgtccctattcacaaggtagaaactgtagaatctatcgtggatcgtgctcgaactcgtaatggaacgaaaccagattatctttccaagtatggatttgtttggaaggaagacatatgaaagatatttcattttatatttggtctgcaatattatttttcgggatcttgattattctttttcaaccggtttgggccgaatggatcaataatagaagaatgcgcaaatatggagtggatgttcttcagattgttgacaaggccagaaaaagacgcagcaaataaatctattgaagtctctagggttataatagcatgacgaataaccctggagacacatgatgcttcctcttttagaaataattcaaaatcttcgcgataccaaaggcaccaacgccaagaaagctgtgctgaccgaggcgttcagaaacaacccagaattggttgatttccttcaatacgtctacgacccgatgcgttcttattaccgaactcaattcaatctaaacgcattccctcgcatgctctcgcgcggcgttgttggtagctgggatcaggtttatgatgttctcgatatgatggccgaacgcaagatcagtgggatgaaggcagatcaagaacttgcgaaggctgcaaccaacatccatccggattaccataccctgatacaaattatactcgacagagacatcaaagcgggaattgccgagaaaggtataaacgcagcattcaacgcagcaggtggtacagggcgtctgattaatatccttccatatcaccgttatgataacatgacgattgacttgctgaagaagatggactttaagcgtggcgtcttcagccagctgaagtcagatggcatgttcgccaacatcatctgtcgttatggaagagacccagagattcgttctcgttccggttctcttattgcgggtggttccgttgataacctgtcattggttttcaaagacctgatctacgatgcggggattggtgaaagcgtcttccatggtgaactgctcgtcattgatctgaaaacgaatacagtgttgcctcgggctatcggcaatggtaagctcaacagtgtcatccaaacgggtgaacccctggaagatcgttacaaggtgatctatcgggtatgggacgtcgtgccgtatgataattggttcaatgctcagcgtgtggacaccccatatgagcgccgttttgatatcatacaacagttgttcgaaggggatgacggactggttcaggttcaagaaacccgtgttgttcattcatttgaagaagctgtcgaacacttcaaagacgctttggctcgtcgggaagaaggcacgatttgtaaggccgcagatatgccttgggaagatggaacatcatctgaagggttaaagctaaaaatggaagtagaatgtgaccttgaaatcgttggcttcaacgaggccgataagaaagggaagcacgccaagacatttggatctctcctctgcaagacctctgatggcctcttagttgttggcgtatcggggatctcagatgagttgagactacgaatgtgggaaaaccaagaggacttcattggtaagattgccgccgtgctctctaatggcgtacaggataaaaccgatgacgccatgaagtctctattccttccacgtctcgttgaagtacgcacagacaagaaagtcgctaatacactggctgaagtctatgctatccagaaagcatatatcgaaaatattttggtgttgttggaagcggcatgaaaattggggttcttcggaaccccattcaataaagacagtagcagtataattccaagaaatcattaccaggtgttattatgaaacttattgataagaaaccaaggaaagacaatgattctcttaatctcataaacattatgagggaattgggagtttctgaattacagatgatgaagatgatgaattgttctgatgtcgtttataattggaccaacggctatcgtctcataccaaaatcttttcggcgttttattctggcgttggtttttattcaccgacaagggaagacagaagagtttcaacgtttcgttcgcacagaggagagatcacgtggttggtaaaaatccgccgtcatatgccaagattggaattgcttttcgaaacgccattctctttataaaggagaaaggattgctgtctgagtactaccagttttgttcagaacgccgccgacttcaaagacagaaacgcaaggagaaactccatgataactgagtgggagaaaatgcaatatgaaagggcattcaatgtttattgcatctacatggcgatcaaattgcatttcaccacaaaagactttgattatggcctgtatggaccgatgaacaattacaagtttgaaacgttctattctaaacagggcgtggccaaacaatttgctaaacttgctcgtcggttcgaatcgtcccaaggtgaagtggtggaaaattatattattgccaactttgttaaatcaccaaagacgtgggtgacaacattacttacaagacaggcccaagaaaattataatgaatatcgccgcctgtatgacaacttctcatataattttcttgaacattttgagagatatatgatccctgagataaaagaaagaggcgtgaattttatccaatatatcaaaggaaatggtaatgggcatccacctttactgacagatattattgttaaacgctatccaatttggtttctcgttggattgaataaagtagtgggtttcatccatttatatgatacaatactcaaggatgacatctattggaactctgagtcttttttattaaagaaaaccaattcagttgttcctgatgaaaacacagaatacacaaaaggaaaactccgtgagctaatccttgcccacggaatttaattaccaatcccagagaatgcgccctgtatcagtacggttggacggctccaactgtgaactattgaagttgttcgtcgtactgactttcttcgagttgtccacattttgttgtactggaaccacaacactggctggcgggttagcataggcggatttaacttcttcgatgttattggctgctcttccttgaaccggataagccccgcccagagtacctcccccaatccctgcgtatgcgtctttcaattgtgaagttctatccgtaacgttgacagcgtctgaggacaccccagaaggcatcggagttaacgtgttgatattggcattgggttgtgctggtgattgactgctgggcggcgtagacgcattctcaggagccttatcgtcttggctgattagcatagactgtcttgcttccgccattttgttcctggctgcttcaccacccagagcatcaggtatgatactcacgaatttatccacgatatcaaacatggcattggttatggcattcaacatattaaagaatggtttcttcacgttttccgcaaatcctttcttcatatcttcaacgactttatttgcagccgccatgccatcatcaatcaatcctgatatcccgcccgttacccaatcaaccaattttatcaattggccttgtaagttgtctgcaagtttaccagggatttcagtcagcgctgtccctttaccgatcccagcaaacagatcgttggatacccaatcaacgatagaattgagccagcgcactggtgcttcggtcatcttcaatacttgttcacggaacgcctttccaaatcctgcgttatcccaaccaaatatttcagccacccaatcagccagatctccgaaactgccaacaagttctgttatacccgcctgaaccctgtctactattgaaacttgggctttgccaaggatttctttagcattgaagaaacctttaccgaattcaaatacagatgttatcagagccaaaggtccgactcgaagcattttggtcgctgctttcaaaggcttcaatagaccaccgatgcccatcccgaatcccatgaataacttgatgaatcctccaatgagttttagtggagacatcaggagtttcaatgctcccaagccgagaagtgcacccagcgccgcgcccataccggaagattgcccgtcctgttttggttcagaaggtggaatagaacctgcttcgggattgttcttgttctctttaccttcttcgccgcgccggaatttctcttcttctcgatatttttgatcactctcatagatacgcgttaatgtgtcattgaggtcttcattcatgtaccccaatttatcatcaatgctgtcaagacgagtaacggtttcgttgttggcatctttattgaagtccagcgtgtccttgtgggtctggagcattatgtcggtcaggtttacaatcttccctcccaccagtgctagtttgtcctgtaatccatcaagacgttctagactatcttcattcgctgctcctaattgatccgatatcatgtcaagatattcaacagacatatcatcaacaggaccattcgccgcttcgccaaatttttctattttggttgcgatccgcatcagttcttcatttccgtccttggaaaggctgacagtgtctttcatccacgtgctatagtcccttgtgaaaataggactaacgcgaacgaacccaccaacgatgtcattcaccgatgggaatttgattggagggactttctgctcctgattagacgcctgtcttgtcttcagagattgcagttgtttgttgacatcagcgagctgtttattcgtctcggattgggcgcgcaattgcttgcgctccatcatttcctttttgattttatccaagaccttgacgatgtcctgctgccctggtgtcggttgattgttatctgccatttgtcacctctcagaccttctgggcttgtcctttctctgttggcgtcggggttgggttagaagccacagtttgtccagtctgaagtggtaataccttctggatcatactgtctttggcaatctcagcgacgcattgataatcatttgtcaggcgtgaaatcttatggcgtatctttgtgacaatatagtggcctgatgataacttacttaactcggcaatgttttcattagatttagggcggttagacaagtcttcaatataataaacctgaccaacgttcaggcggttatcgccgaccacgagcaaacgcataaccgtagatgctagactgaattctatgacgcggcgagcataatctatttgttcagccccatctggttgtgcctcgatataacgaacattttcacgatcaaattgatccgagaataacgggaacgggtcaaggtgagccgtgctgtcaaaccattctgaataaatgcgttgggtcgctgtcgtggattttgtactaaagtcatacacgcgctcgttcactgccaagatatcccgataattcgccatgtactgatcacgcgccagtttcttttcagccttgatgatagtccgcatgaaacgttctgagttgaaattgttatcctgcatcaacggcgcgtcttgagggtctcggaagaatttcttttcaatagaagcctcttgcttctcttcagcagtcatagactggttgccctggttgaacaacgtcgtcatgcttttgaagtgataacccgtaaagtcttcatagaaaacgaacggcatgaataattcgtcatatgcacggctggccatataatctatggagcggagtacaggccaaagaggggtagcgaacctttcttgtatcccatatgaaggatctatgtcttcaaatttggtgcgtgaattcagttgctcaaagatcttagctgccatctcagaataagaaccgcttaatccaagagatttacgcaacatgctatctcgatacgcgtctgttgttaccaagtgtaaccagaatgcctttttcgaagaagagttcgattcatcagcaactctccctacccgactgactcgcaatgaaagcgtagtgtaatcagaagctgcaggtgatttgaacgataccactacttcctcacctccgaggattggcattgtatccaatatatcccagccttctttgatcagaatattagctgtcagcgacggtgaagcattcccttccagaccaagatcctgatatacgttgaattcttgaaacaacgatgaaaggtcataaggcttgggagtaccgccttcaggcgtgtgcggtaatatcgccatatatttcaaatcaaatgttgtggacggagttaaaattccgtcctgggattctttattttcgatcatttggtcagctcctgttccagctgattaacaaaggaagaaacataatctgggtctagaacctgaatattccgtttggcttcgtttttgtttatagcatcatcatgatatgtaatacctgtcagaccatagttggcgataatcgtggaatcatccatagaaccaaggccatatgctaaacgtattgctctaggatctgtttcatgaccgaattcgtcaacgtagtatttcacatcccacattccgtcaaggccgtatcgagccgtcagttcttcaatgatacggcgctctggttttggccagtcctctgtgatgtccatgataccattgataagacatgggatccaaaacagctcgaaggaaccgtagacgcgttcggcaaaagaccttggggtttccccatcaaaaacagtatacggcaggagaagcccttctatgtccctaattttcttaacaaccataacccgtcgtgttaagttctgcaacaggacttggtcatcctctttgacaccaattagttgatgccacacgagtggaaatttctcaaaatatttcatggcatcttctccttaaaagctgtcgccttcggtatcaaagcggcctttgtgcaacggttctagttctataaacgtcatgtcgatttgtgtcgacacgatgctgtcatctttgtgtacggcatatgaagagtcaggcgtctcgttgacaaacatatttgataggacgcaagtcgatatccgatgcaaccatttgttgcgttctccctggaccatgaacgtgatatcaaacgtggatggatgaagatagaatgcgctggaattcttgttgtatttgtattctggatacatgtgcatcttgaataaacggataatctcccgcaccattttcgcctctttttgcgatcttggagtgaatttgaatgtgaacgggatttctcggttgcgcaccccttggaaaatcatttccacatacgggttggtcatcgtacctgtaaacaattctaatgtatcatgagcattaattgtcggcaagaaaggaattgcttccgaagcagattgaatggcctttgttgccgcaaatctccccatttctttcccaacattcaaagcatccccaagtttgaattgactcatatcttgggcggcgcgtgataagaattgggcacccatacctgctaatcccaactcggagccgttccagccaacgccatagttggttgtaatggattcgggcatacacaacacaattgactcgttagagcggacgtgacgcgcccaagcatatttgctaatagaacccgatttagaaccataaaccacaggagtcttacccaatggattttgtatcggattttcgacggtttgggttgtggtgtccccatatgaagaaccagatattcggttgatgttgaatagaacataatggccgagggttttaccccctgttatgtctaatggatagaccaattgcttctgagacttggccaagccttttgtgttcagaactttgatcttatcgatggtcgacttgaaattcgccatggtctgaagccctttaattagaatagagtaatgacactatttaggactagaatatggctatgtatctcgtataccaaattaccaatctgataaacaataaaatatatgttggggttcataaaggaactccagatgatggttatatgggttctggccgagttataacaagagcgatattgaaatatggtgttgagaatttccgcaaagatatattgaaagtttgtaaaacatcagaagaaatgtatgaagaagaattcaggatagtgaatcaagaatttatagatagagatgacacatataatttaacatgcggtgggcgcggttcatttacgcacataaattcggatccattaatcagggccaacgcttcggagaaggctctcaaaaccctaagtttaaaatcaaaagacgaattacaaagaatttacaaatctaggggatttcctggagaaagaaatttttggtttggtaaaaacaggtccggtgaaaataacccaagatttggctgtgtaatggaacaagaaacaaaggacaaaataagaaattccaacaagaaaagagcggaatccggcctcgtggattattctaattgcaaagggccaatcacaaaagaagggaagaacgcaatatcccaagcgaactctagagaattcaaatttgttaatccagaaggaaatttggtcacagttatcaacctttccaaattctgtaaagaaaatgagttgagcgaaggatctatgagacacgtccataacggccgaaacatacaacataaaggttggaggaaagcataatggcatcgtatttacaaggaaaatttattcccaacaatcctcaaaaatatgtcggagacgtaaaagatatcgtgtttcgttcctctttagaattggttgcattcaaattctgtgacatgaatcctgctatcgtgaagtggggttcggaaacttgtgtaatcccttacatatctccggttgatggccgcgcccatcgttatttcatggatttgaaggtttggactcgtcggcaggattccgatgaattacaaattactcttattgaaataaaacccaaagaccagattaaagaacctcgcaaaactaaaacgatgaaggaatcaacattcaataattcaatgcgcacttggttggtaaatcaggcaaagtggacagcaactaaagaacattgcgcaaaggtaggttggaaattcattatctggacagaagaacatttggtgccaggtgaagacccagaagttaaaaaacaatttgctcttcgttctaagaaaaagcgcgaagtcgaaatggaagatcgacgccgcgctcaaaggattaatgcgttgaaagagcaaatgaagaaggaaactgctaataaacaaccaacgcaatcggaagatgatggtttgttattaccctgatttccttggatgtccaggggcaaagcaagtttgtttgtccctgcatatcatccacccatcagcacgggcttgtctagcacagtctccccaagattcaccagcataagacttcatcccacatctggggtgatgttcttcccaagcccaattacaagatttgcattcaacacactcacaatacaaatcgacggtgtatcctgcagtaatcatttagtcacccagatgatcataaattttttgacggaattcgttccctttagcacaacctgtagccaacaggcgagtaccgcccgtaccaataacactgatggtcgaaaacttcaggatacggcctgtaatagactggtccaccttaatggtttctactttaccaagacggagttcatcagcatcacgacgaataaatccccgtttgacaataacacgcttgttggtgactgcgaattcagttgtcaatacatttaaaattgttggtatcagaaataacaagctgaatccaaatgttgggataatcgttaaaataaccagaacataaatccaaaaaccgctccaccaagtcgggcgggtgaaagcgatgacatgttcattttcgccgagcatgcggtctacataacgcattttaattccttactttgtctgggttggataagatttgttcgtatccgacttcgccttgatcgtcgaaaacgtgtttaaggattgccatatctcccaccatccggtaaacttcaattccggagttcatcaatcttttgattccatccatgatacgatatggctcttgataatatactttatcgatcttgccattgtcgatgatcttctgagtgcaatcagggcatgggctatgtgtcacaaacataaccaaccctgtgaaatcatctgcattttcggggatacgcattagagcattttcctccgcatgaatgacacacgggtttgtaacaatttgcccgtgttgttccatctcacaaacattcggcatgccaggaggcgttccgttccatccgatagccacaggctgatcagtttcagggtttacgataacacaaccgacttgcagacgccgagcataactggttacaccatatgctgcagctgatcgcatatgggcgaacatcattcgtggtttgatagccattattttaccagagccttttcaagtaaaaagatcatgtcatcctggtcggcgcggccaaagatctgaccgagtgacaccatggaatgaacctctttgacaatcaggtctttgcctgttacaccgctgctgtccaattcgagaattgtagacatgcgctcaaccatatgaggtggatgattgatataacgttgaagatacttttcacgcaacgcaattttttcgataagatctgacatttctgcattctcttctgaatacagacgttctttagatttcagttcttcgaggcgttcgtacatttcttttaatgaaatcatgtttgtctctcccaataaattaaggggaagtataccttcccctttcttcaataacctaaaatattattcagtacatgctttaccgaggtctgtaagcctatagaatgttacttcagacttcggccaatgcttatactcttcagagttgtcatgaaccattgtaaacgcaggttctatcagacctttagacaacaaggattgacctgaacgcgtatcccatggatcaccctttacagaacgaaacccgccagatatcggtggcgcatgaaaagtccaaaccgttttccctttatcgttcgtccaagatttgggattgaaataccaatgacaaaggcgtctggcttcacctttttcaaaattcaacattcctaaaaggacgcagagttgatgtcctgtgatgcgtggctttttcataagatccccaagtcggtaaggtaacgataaaacaagattatagacagaaatccaattaacgcccaagacgacaggataggcttgcaatttagcgtagtcttcccattgggtatgttaggacttccggtaataacgacggagtctgttttggctctcagcgtcatgatagaacccatgagaatgattgggcgcacataatacccttcatgattgcgcttcacgatacatggtgaagaaaatcgcaggaacggatcaccgcttttataaccaaataactcgaccttttctatttcttcgggtactttctgaccaggaaagaatattttcataatacaattcctaaatttgtgtataacaactcagccagggttacaggacgcttgaattgttcccaactgcagttcaggtattcgcgctcttcgcgaatattttcgtgaagatgaccatggatattatacgcccaacggtctaattgataaggaacatgtgttgtcaacacacagccaacaggcgtgtcacgttcgtacatagcgccgaaggagctaatccagccgtctagatacagagacaacaacatcttaaagctgtcatggttgccttgtgccaccttgatgataaagttcggacgccagtcatcgggaacggggcgtcgcttaaactcatcaatgtttcgcttggcaccctcccgcatcaggcgaatgaacccttcagccccgatgaagcatatatcaccagccagttcaagaacgtcccgagacttcaacccctgaaagatactgtcaatgaccgcagcgtcatgggcttcctgtgtatcaaatccacgcggtttaaagaccttcttatgcccaagatgagtgtcaccaaggtgctttacaattcccgccattatggtttcctcagccattctaaaatcggtacgttcacaaaccctttatgccaacgatgcggtttgtgacctctattatgttcgagcaaacacgtgtctggtgggacactgaagtggatgccgtcgatgtcgtagtaatgtccatcaataagagtatacacatgaccttcaatctggctataatggatctctgcttgaggatacacatgcttcagaaccagagcaacctgatagcactttccgttcttcccaacatactcaacatccacctgttgtgggaagacgtctttgataagatttatcaatctcgtgatgtcatttacaacaaccgaacatttcatctctatgataactcatggcgatgtcgaacacaggcagggcaatcacaattctttttcaaaattggcaattcacaccatgtcattaagtcatgtattggggacatgtgatgatagaaccaagatttcttgagactaatcaatggatataaaatttcaatagatctttccccgtctctggtcatcatgttgtatccttcaataactttattaaaggcgggtatccagtgtattgcgtcatcgccgttaacatatgccaagatatacgtggacggcaagccactgcctttaaattgcgtgtttaataaccatatcggggcttgtgctaaacaagcgcgaaaaccaccgttcgggacgttgatactactgataggttcttgatgatgtgtccaacgatacatgagaagtggggcatttttattaatccagttgtgtattttattcctcgcccaattttcgcgcttagatttaaatttattattttcgaggtggcaatacacagtatgaactgaatatcctttcgataacaaccaaatcaacatgtatgtgctatccaccccaccagaccagaacaacgtcacttcttgattattctccggcaatggagcatccccttgaagaacacggtatacaaaatcacaatcagatactgagacaacaatttccattaaaatattcctcttaaagaatcatttatcctgcgcaattcgttttccacttttatcttcatatttttatcgtcgttcacgaaaatcattttatctaaatcttcgccattaatctttgtataaaaataaatgtcatgctcaaagaaaatcgtttttccaacagaacgcatggcaaagaacgttgcgatagttcttggtaaaggtttgaatttaatggcattgaaacgcaaagcctgatagagaggttttaatgttcctaagcgaatacaatgctggataatttcttgctctttcatttgttgtgtgccataaagatatgatggtacaatgataatacattatacaaatagaaaagcccctttcggggcttatttgtcttcagattcgtacaaacgccaattgccgatgctaaaccattcccctacattatttgaatatctggttgttcggactcctccgacttcatacgcccatttgaagaatttccatttggacgataaagaaggacattcgcttccggatttcaaagaacacgctgtgagccaacgcgtaatactgagagcctgaaaaatcggatggataaatgaaaaatacaatatccaaagcgttatgacaacaaaaacaatggtcgtgatcaccccgacaatttgaaagaacatttaagaacctccaaaatattgaacacagactatgaagaaaactatgattgtaatcaaccatgaaaacaaggaaagattcatccttccaccttcagccccatcaggtcttccatggttttctgaatgcgtagccagtcaagcgccgcatcataacgagggtcatgatagatcatgccttccagttcccacgccttgataccagcatatcgatcctgctgcccaacacccttcagccagcttacgatgtcaaatgtgtggtgatagtcccaaggttcgttggaatcttgcttacacgtcacctcaatgatgtgctgagccttgcggaggtcaaacagattcctatcacaccagtcaaccgagcgcgggtcgatcccaaggcgatggcatcccttgcgaatttcatcggccaattcgaaaatagaaatatccttgtcggtcggatatagactgatagctcgggcggcgtcgcacgggttgcgtttacctgtacccagccaccattccacagtaccctggtctttcacacggcctagttcaatctgttctttgacattcagtttaataaatgtcgtgcgctcttcaacaagctgctgaagggtataacgcttggtcaggtcggcgatagtctgtgcccaagacaacatgacagcatcatcccaacgaccgagagtttcgctatcagcgacggccaagaaattagatttatacatcagtttcccctgctttctctaaaaggagtttcaaaggatatgtcccacaagcggtggcatactcgactttgaaatatgaattatcgttcatggcgaaattgcgatgtgggaaatattccccgtcatccaacccgcctaatcccccgccgacgcgatattgtttcaactcgacttcgccgccataacggagttgggcatataggccgataggagcgcgaacataatgctccagccatacccaatttccatcgtgtaaacgtgtagggataaatgcaaaacgcaccttgggatgaagaacggctttggcgagcatctcttctgttatttcacgcttcatccaataccttcctcttcgatgatatcttcttcactagagacgacagagattccgtagtccaccatctgggaaagttcttgtaaatcttgttcaaacccgtcaccctgtcggatataaaagacgggtacgacacgacgcctggtatcttcgaagatagtatccgcgattttattaatctggctcactacatgaaacagtttcaatccaccgataccacagccgatggcgggtaaaccaacatagactgtttcgtcagtttttgcctcaatgatatcgtgaacttgttcaaacataatctgaactgaactgataatggaaggcatgcgtgcgttaggaccagggtagaactgagtatacaaattgaatccccagacaccagtgtcatgatcaaacgcataggacatgttccccaaacgttgttccggaccacgttcatctaattggtcggcgcggtaaatttctgggaaatccaatcgaacatggttggcgatacctgcgcccatcacagaccaacaatttgcgccatgagcgaaagcattgatgagattaaaactcgtagcggctttcagtaaatcgccgtttgtgataacatatgtggtcatgcgtaacgtctccattgtataatttcgttgttgacacagctaacctggatgatacacatattctggcgatggtcagtgagtgtcataataaaactgtctgtctgtgccataacacaattgacagatattcctcgcatcatcaaacgaacacagatttcttgatataatcgtttcatgtatcctgaaactgaagcggactcatacacgcgttcaaattcatataactcgttcatttattcaggccatcaatagcgtgttcagaaatatattcaacaaattggttatcagctgccatctttaaaagttcaaataattccgttccaacggtactcccaaacatcccttccaatacaccctgaacgtcttcatctatttcttcgatgacgagtccatacgatttcaggcggcgtgaaatagcacggaactgggcaatatcaccaatttcatttgccatgttatttgtctcattatggggttaggcggcaattataccgccaatttattcaatagaggtcttcggaaaagagttctttgaaaggagtgttcttcaaacggacctgattagtttgtccggcttcacgggcgaccacagcggcctcttcacgggacatccattgcccatagttatcaataaacccttgttcacgagtgtgggtggtttcgataatacctgcttccttcaatcgtttgagttgcgtgttcatcaatttgtcgtgatgtcgggcgcttacaacgatcacatcaccatatttgttggcagcagccacaacacgccgatcaaacttggttgggtcaggtatatcacgagcatacaaggcgatgtgagtgtcgtccacgtcccctttcacttcagagtataattgacacccattaggagatccagtaggcaaagaacgaaaatatccaagtaatttcataatctaatctatccatatttcaggttcaggaatatcgaccatctccacggtttgattctttaattcgtggaaacaatctccgaggaattgtaatttaccatctgtcaaaaataaatggcaacggttatttggatcagaatgattcaccaacagactaggagtgaaagtcggtgattcgagatcgtgattccattgccatattgggccaggcatttgccctgaaatgcgataaggatgaagcatattacatccaggacaatgaaaatatatggaaccacccattgataataattttggagtcaataattttgacatatcacgcctccacttctatatcaggattgtatgggcgtaattctccaattaacttagggaataatacgaatccaaataaattaaatgaacgcatggtgccaaccatacccacttcatccgattcggttacatcagaaagagcgcaaagaggtatccataaatcttgctgaccttcatgattgacaaaagcaacttcctctttaataaaacgagggcataagatatcgaacaaccaatagaagaaagccagcaatgagagactgcggaaatatacaaacatggtgaacccccgcttgtttggagggaatttcaccctccagatttataggcccaatgccttttcaagatcatctaaatcttcagtataaagatcgatgtcttgcttattatttaattcttctcgttgaacttctaaattctttatctggttcaacaaacgttcacgggcatctagtgtaattgatgataaagacatcttgaataaatcatcgagatcgccatcaaaatcaggatagtctgtctggaataacgctttcagttccgtgaggctccggcgcatatctgtaatgacagcccacccaatgaatatggcacgattgttcaaacggtgtattttgtcggtcaattccttgataagatactgacgtctatcttcatagcgactcaggcgataattgaaaaatggaagcaacatgtctttccaaccatcgtaacggtgaataacaccatcttcatcccaagctgtccacacgggcttattagctgagtagagacggaacatctgttcaacctgttcgtcattcaacttagacaattcaccccgcttgaatacaacagtgatatcccaaccatcttcggtcgtatcgttggcgtactcagtgaggactcccgccttgtacaggggcaacagcactttggtctcataggttttagagaaccaaccaataggaacctcggtgatgttcaaagtggttgcattaacgcgggtgaacaatccacgactgtatgctcttccttcctcagtatagcctgtctctcctttgaaaccattccagtacggttttaagtccttcgggtcttcgccacggagaagtgctctcagggcactgagcacggacttaacgcagtgacatggggtgtcggtggcataacccgagccgataccattgataccattcacgagaaacatgggcaggatgggtaaaaagaatttcggctccagtttctcttccccaagataattgtattccaatatcccatcgtcctcctttcggaagattttacggataacttcagacactgccactgaaatataacgagcggaagacgcttcgcgccccatgattgaaccaaactgtccgtcgcggtcaaagtatgggatgttattcgtacctgggaacccctgagccatgttcacgatcgtaccgctcatgttctcaccaccggatttgtaattcgtgcgagcggcggcgaacatgccaaggcggtcaacaatctcctccttgttgtattcaagagcagcgaagagtatcttgcgctggcttggcttcaggctgtcaatcagctgagggatttgacggatgctgttgaccacggaaaactccttgtggtcagtgttgacaaagtccgttacagtaatgctcttggcaaccatcttattcatcctcggtttcaaataggcaaacatcactaaaccaggttttgcggtaatcggcggcctcatcaccgaagccattcttcagtgactgcttgtatccatcgtccagcgtgaccgtcgtagtatacgcatccaggttgtttagaatacgcttgaagtcttcagtgctgttaccgccaagacctttcagatatttcttcttcgtgatcttcttggcgtcaggtgtcttcaggaactcctcatattcggcattgttcatgaattcgtgcattatattaccacaccacacgcgcatgtatggagtacgaaggagcttcagcctaccctgacgaacgtattcaggccagaacgtacaatacagagttattaacaacccacggatatgaatgccgtcgtcatccgcgtctgttgccacgacggtctgtggatagcgactgatatcaagcggctggcctggcacagcaccgccgtgaatggtgcataaattcttgaattcttcgttcgccatcacctttgagcgcggggcgttcaggcagttgatgaacttaccacgaagcgggaacaaaccaattttcttggtatcacgagcgttcaggatagggttggatgcgctatcaccttctgtcagtagcagcgaacacccactgcggtcgcctctggcggtcgccggataatacttctcaatctcacgataatcccgtttgctgatatccttcttcgccttttcaaattcggcatcttcttgttcattgcgtaatgaagccagttctttactcagccctgctgtcacgaactcaagcgccttgcgaatcagtttatcgctgggcttgtaggacgtaccaaactggctcacaggagtcgtcatgcgctccttggtctggctgtcaaagcgagggttgttgatgtcggcggcgatgaacaatgacatgtggttcttgatcatcgctggcttcagttcggtcttgaacttcttaaccagctgagggcgtatcgccgccacgatctggtcagcaacataatcaacgtgaggtccaccgatgtgcgtggcgattgagttcacgtatgatgcatgcatgaacgcaccagatgagggagtgatagcaacagaccaatcatccgtttcatcaacagccgagccagcgtagaagtaatcaacgaaatgaccaaatcgatcaatgcggatttgcttgccgttgagaacaaccttcaggcgcgggttgcatgccgccacttcgaatgcacgacggtaaatcatgagcaggttgttctggtcaagccccttaacacccaggcgcgcataatcagggatccaggcaatagtggtgccgaactctttcggtgtattgccgatgaccggattggacttcttgctcatgttgtcttcaaacagttgagtataagacttcttgccgtcactggtagcaacgcggaaccactttgagaacacgttgacgagcgaagccccttcaccgttctggccgccggacttctggttgttgtactcctcgtcctcatcgttgaagttgctgcccgcatagagcgagccaaacaacatctcagggagccactcattggtgacgccatgcttgaccacagggatacccccgttgtcagaaacgatgatttgaccgttcataggggagaccgtgacggtgatttccgtcaagcctttaccctcaggggtcttgctgtggtcaacacagttggtgatgatttcatcaaactgtttgatcagcgcaggggagtactcaaagttgtcacggaagatgactttgtctttggttgggtcatacacccacaccgtccccacagacgaacggatactgcccagatgacgctctgggcgaagtaggatatgctctatgtgagtgagctttttgtatttgcgttcaatatcaattttgttggacatcggcctatcccgtcaatggtaaacacacaagattatgtatggggttggcttatagaccaaatttagtgcgccatttttccactttgtgttctatggcatgttccactttaacgatcgtgttgatatggttttgattatatccacacgccaatttggcagtattgcgtatcagatttgaaaccaggtccaccatattatcttctgaacgttgaggttggttcagtaaacgagtgatgcggcctttgtaatgattaaccatcgcaagatatttgtctatagtttcaacaccactgcagaaccaataatcctccaacgtatgttggctgagatcagttgtgtgattggccgtagccaaagcaaacaccatggagtcaaagatttcttctttggtcatagacccatgttgttgaacttgttgataatccacaacatataatagatccagagccgagatgataacgtccgccacttcaccgttcaggggttcggatgcagcgattccataaagcacatctgatagttcacaagactcttccacgagtttgcaatatacaaacacaggagaacgattaggatcgcgctggctgagttcatatgatgtgttgaagatagtttttagaaaattcatttcaaatatccttctagacggaagcggttaatatgctgtatgataaaatccatagagttttgaaaactgtatttcccctgatgttcataagcgtgaatcaggggaagacggctgtgagccaattggaatctcaacttggatggcattgtcagcaatttttgactcgtcgctgcgataatgtctcgtttaaatttgggatcatcaggggacaatacaacgatgcgtgacttggccagggtttgtccaaattgcttggcgtcgttcccataaagactacgcaagtcgtcgtggcgatattttagccaataccagtagccttctattgagttgaaattaccatcaacagtatccatgggatggtatgcaaaatgggaaaggaatctgcctagttcggtgcggcttcgactgtatatgttgtaatgtgtcttgccttcactatccggcgtcatacgttcttttggaacattgagtaatgggagcatgatatagggtctcaaatcaaaattgctgtgtgtaatcaggtcaaatctttttatatgctttcagggtacagataatagagacgccgccaaatagaactaacaatatgaatcgttccatcctgaaatcaggaagggaaagcagtcccacaaggctgtctaaagcaatcatgaacgttactccagtcagcatgcccaacttaaaggagttggaacctcttttgcaacgtagcataaatcacctccgattcaaatggggagttctctccccacacagatatctagcccagaaacactactaaagtcaaatttgtgagactggtccgagcatcctgcaacatttcacgggcagcggctaaacgttttacatcatgtagagtgtcagagaacacgattatgttagccttatcgatcctggtccccatgaaatgggtgattgtcgtcgtgctctctttcttgtcgacatcagggataagtaattcttcgcccgtctgagggttgcgggaaggatagaagaatgcatcacgttcacgagaagtatggaagacgccaaacacatatgcgtcgttttcgacctgaaactttttagaaagtagtttcttcagagcggctgtatgtccggtctggcggagcattctgtagtagagcatgttgccggagcaaatagggtctatcactcccctaatttgacgttccttttgtatctcatacgcatctgccacaagccgctggatcatatcgacatgggcattcatggatatttctattttacgtttgatcatagattgcatttggtatatcctcagttaggttccgctcatggggaacgggtccattatacacccaatgctggaaatgaaaaagaggccgaagcctctttaatcgtcgtattggcctgtcctgaaattccattttcgcgccttggataaatccaaatctttgaaaatgctgccaaagatctttttaaaatcagactcttcttcaagatcttttggttttttgacaggctccttaaccataccgccttcgggtttccccacatcggcggtcgtggttgccggagcagaagagtcttcaaggaaatcctggaagctcttcataaattacaccacagccatttcacaacgagtagacgtgacagaatgtccatctgcatcagaaacaacacaccaatattcgccttcatttgcggcttcaactgctgtgataaccaggttagcagtggcggcggtggcattagtgccagcatcgataactgaccccgcacccccaggagtatcagagaaatgccacacataagtgtatggagttgtaccgcccgcagcaacaacactaaaggttccgtcttgtccagaagtcacgtctttggtggcaggtaaatcagtcgacagcgatagagcgcctgcaactgtaataaccgtagaatctgtgtgattcccttcaaccgtgacaccataaaccgttacagttcctgaagccagcaatttgaatttaccaccaccgacatgtacagctttcgtaatatcaccagaataccaattaacgccttgatatgtggcattggcaggggataccgtcgctagcaattcaacaaattgtccgatctctccgctggctgtagctggagtaatcacaagtccggtggttaaaatagcattcggatcgtggatttcataagatacttgaaagccgataggcttggtgggatccactttggtgacaccgtccgcttgtaaattgctcttggtgagcagagcgtcaagacctttacattcggtcaggatctcatcaggatcttggttgaggctggtcttttgccaaccctttgcaacacccgtcacgacatgattgcgttcggtttcgggtttgttgcggacaacttccggtgcgactaatactgtgattgttggcatggtttcctccactcatccaaatcgttgttcagagccatcatcagctcattatctggttcgtatgaaccggaacataaatcatctcgccaaactcctaacgtcaacagcgctgctttaagacgaggatgttggttcttatccgttttcaacaccaacattcggcacacggcttcggtttcaaaggtattgaaaagaataatcaattgattgataagtactcgaggacttttccctggcacaaatcttttcaatgattgattaattagcgaaattcgctgaatgtctaccatcaaatcatctcttgagacaccaggggtgatataatgtcttagagcgtactccatcaaattgctttcgtcaacgatagccatattgaattataatcagcagttatacactgggattatttaatctgcagtttttcagtcaattgcgagattatttcattacggtcagtcgcgtcgatacccaggggttccgcaagcgttttaagatcattgatatcgagcaaaagaagaacttcaactgtaatatcggttgataccacagtacctggtttcatcggcttaccgaatttatctaacccataacggcgcaaagttccggcaagcccggtgagttcctctagaagctccttagagccgtctttatattcacacacccaaccgtagtcaccagcgtagacaataccgccgtcgccgccctgaggcacgttatccaccacgcgttgcagataactgggaagtttattttcttcctgtgcagtgatttcttggatggtttgggtgttttcaattttcttttgtgtcttcgccatgatcagttctcctatagactgtaatatatttaccacaacaattgcagcaataaacatgcatggaatatccgtcgtcgttaaagtcacagccgccacaacgacaaaataatttcacgaactggtcgcctatgacttctctcggaggaaaattcctgaacgagtattccttgagttctccatcaaattggaatgaatcaacaaggaactttgtttttcggcgcagtgatccaaatgcctgttgctcatacacgaagtaaacatctctggtttccatatcgacctcaaagaaaaggaggccgaagccccctcttttatgtcagttcgcggttacttgtatagaccttcaacatcaatggatttatgttggcggttgtattccgtggtgtaatcgtctaataccagatattcacagcagcggattttactggagccataatcatcagtgatagacacgatatctttcgggttcagtttgcaacggacaacgcgttgtcccaggctaccaaaacattgtttcagataagccagagaacaaacgtgaagaccgtaagagcacagatctttgttgttatcgttcacaaatgagcgagccattcgaacaatggtgccaggagcattactaacctttcctgtgcgcttgtccatgtagttaccgcgaacggatttatacaggatgatatcaccgtcttcatgaatttcgatatccgaatatgccatgaattcatagattcgaccggactgaaccagcgcggcgctcgggttttggaacattttgtccaggaaacgacccaagcgttccaagttcggataatcgccttttaatgccaaagccaaaatgcgtttggcaacggaagttccggtgatatcatagccaccccagcgaacacgattgtctgaaaggtcaaccatgcctgtggtgaattgtttgatggcttcacgcggtttcatcagagtgtacgctgttttgacgtcacgatttttaatcgcttcaacaatacggtcatacgcctgatggcttgtggttatcgtttccggtttgccatccataacgataacgatgctttccggtaaaatcatgatctggtgctcattcaagaacactgacagatctttttcgttaaatgagttttgagtttgtttgtcgccagactcttcctcaacagccgttgtcttaaccgagaaaacaacagacgacaacgccagtcgaagtgtacgccccagaattgcgtcaaagtatcccacacgccgacgggcatcaagggaaccgcgcatcgggtatgtttcgccgtcatgttgtacactgattaccacgccgtcattgactttggtaacgccatcaaccagtgctgtcgccaattcggccaacggagaaatgttttcttcattgaagcggcttgaccgagtttctaacggtttcagttcttcgcttctgaaaagtttggcctgaatagcagcagaactatcgtaacgcatacagataaacactgtttggttttccaggaaacggccaacataccattcttcgccttcagaatcgacagacaccgaaccttccagaacttcataggtgtcggggattttcccttcttcagtcaattctttaattggcgaaatcacgaccacgttgttagattctttgactatacgaaccatcagggcgcgggcaattgttttggcactgacgccataaactgatgccagagaagaattgctttcacctttgctgctcttggtttggatttcttggatctgttcagtggtcagggatttaatcgaggctggatcaacacccaactctttatccgacacgcgttgtatcattgagcgcggtacgcccaattcactggctattgaactttgacttctgttctcattcaacagcttgagaatacgtttttctatagcattcatcttgtagttctcactgtggggtaatggggctattatgccccgagaatacgttttgaaatagccttcacaaatctagtggcatcttcaatcgcgttgtgtgccgaaggctgatattttgacatcatgaacttcaccaaacgatgacattcaactcggtatcggtttttggccttttgcttttgagatatagtgctagtgacctttatctcatcaaatccttcagcagttaatttttcattcaataaatcaacaacttgagttaactggttacaggcgcggaaaaatctcgtagttgggaaaggcatttctatacgataatgtaattcctcaacagccttgcggaaataaccgtaccccgatccagacgggtttcgtttggccagcatacgaacaatcttccattcttcttcagtgatttgttgtttcgcataaccgatgattgcttttgccctgtgttgatgatgggcatagccatccgtaacttttccatgggaatcaatcaacagcttggcaccataatcaatgcgatgagacatgatgtttatgcccttgcgaagtttcaggagtttcttagattcaagcgcctgaaattcatctttataatgctcaagcaacatatcaaacactgcgtcaatggggatgaatacttcaggaaatttcttcatcatcgggactgatttacggcgagcaagaacgattgttggatgtgctcccagcaaatcaaacaggttttcgtttccattctcgccgaacttcgttctttccatcgcttcacgcaaatgtccagccgtggattgtcgtaaggtattaccatgaattttatggataccttctcggccagatacttcgatatatacgatctttttgcccgaatcaataatatctgaaacggtttttccatctaattcataaacgctgtcgaagttaaatgaattgtaatggaaactaaacatcccaggatctttttcaacggtcgggcgaacatattccatttcatgcatgaaatagacttcatccggattgaataaatcgccaaagacctcgatcagtttatcggcaaagaccaggtagtccggcgttgtttcttgtacaccgatccatgattcaaaataagacttcaattcctcatggttggtaaatgattctttatgtgccgccagctgattccgattactgaaataattttcattcggtttgacgacgataacgacattgaagttattgcgtgctgtttgtatcttcaggtttcgagcacgattcgagttatcaataataacagcaatttttgatttagatttcaaccaatttccgacagagttcacataaatgctgtcgtgtactacaccgttgtcgtgtacttcaatttcgtaaggactaccaatagaccggaaaggtgcatccaaagagtaatcgcgcacgacgtccggcgctccgatataacggaacgtgcctttgtagaaacggttatctgcaggaaattctcgctccactaatttacggaaaccgctaccgaacatatcagtcagctgagtctttttcatatacaacataagaggacccattgactccgcttggcgaagttcatacatcttcgcttgcatgatattttcggccacatgcttgaacacgcggttcaggctttcacgagtgaattcgtcgtatgacaagtcttcgcgtgacggcggtacattcagttctccaagttcgaagaaggtataggaactcgggaatttttccagcacggtgcatatttcagagtccaattggtcgagatctatcgggtaagaaaccccgcccatgacagcatagtggatgttgtcggaatctgacttcggttggatatatgtgttgcctacacggttttcgaagttgatgtcagaccaacggaaagaataactggcgttttcaaccttcggtcgcattgaaggtcgcatcactcgacccaaacggaccaactcttgttcaaaggcggtaaaacgctgattctttacaggaactttaacagtcaggccgttgaactcatcagtgtcttccggtttaggatcacgggtatctaaatctttggtaatgagatctacagttgggattctgtccgcattcagatacatcaggaagcgatacagtttcccttcatagcgactttccactgtaaatgaatcagataccgcaaatggggatttagaacctagcccaaacgcccctatttgcccgtcgtcgtcctcttttgtactatggaagagtgttgtatacaaaccaggtccacgaatgatctcgccatcctcagcgcggaataccagtaccccattataataaccaggtgtaccaataacagcagtgctatcagggatttcgtcttccttcacgacgatattaccttcaaccagcacttcatcttcacgcgctgtaataggctcgccgatgatcatttctaatggaagaccaaccccataatctttaatttccagccagggttccataacatccggtaaatgtacaacaacaggagttcccttgggggcaagatgtttgctgtatcgttgaggcataggagtgagaggagcataatgcgatggcatccagcgttgttggcgatcacgcatattatgcgagtctataccattacacaacgtctcacgcaaagacgccgcctctttatatttgtagagacttgaaaataatgtttcaaataatttatccgtcatcttgacattgaatgtctgcccgacgatggtagaagaagagcgcacgaaatgctcggacttgcgcattttcatagtatagaattcctgttcagatcaaagaagaacacattattgggtaaggttatgatactggcgggtggttcaatagaaaagcctccaatcggaggctttaatcaggattaacttccctgaacgtttacaacggtggcgttgaaattgaatatcgggtgggtggcacctggctctaacgtctcttccagataacccacaatattgcgtgtcacagatttcggctctgcgcgggaaggcttcgttgcgccgaggtcaataatgctcgaagtgacttgttcaacctgagtgtgattgacaacaaaatgataacgactaccgaaggccgcaacttcaacttctgtgatttctaaaccactcaattcagagctgttcaactgcttcacattcaattgagagcggaggtgctctacgatattactgtaaacatattgtggtgttatcagctgttcaatagaactcatgacatatcctcatttctttgggggtttattgttacttaaacggatcacctctgctcgaaggaagaggatttcatcggccatcattttaccttcttgaaacataacatgacatcgtgatgatctgggttcaccttccgataatgcgtgaaggcgatattcggggatgacgtcatttacggcgtttctttttgttctttccatgggtcttggcctttttaggacgactgacggaaggacgagcatgcccaggttccccaggggtacacccttgtgcataacgatgatcatcccagagacgaccattatgcgtcatatcttcagccatggcggacaaagcaagggttgctacagccgcagcagataatggtttagacatttgtttcaccttttgctgcatacgacagagcttttacgatcataacagccaaaccgataggatcgtatgcattgtcgaattcttgttctatgttctgacgaatttcatcagaagattgtacttcagagcagttcttactctgtctgcgttgctcttgctcgactagatctttgaacagaaggtctttgaaagaatactcatcgaagttttcaagataccgttggatgcttggttgttttacatccatcgtttgacgcaaattgctaatagttgaacttgcctgttcgcggtgtttgttcataacagcaaagttaacgggcttgccccaagcattcatcactggattagagcatacttggccaaagagatcttggaaggcatcatccatgtggatcaacgcagtggccaattcttcataagttggtttttgagtagtcattttcgtttgctccgatttttatatttcttagcctgacgtttatgttgggcggctccaataattgcaatctttcttggcttgtcagacataatacattccataaaaaagaggatagagcaataataccctatccccctttattgaagaacgattagtctttggattctactttatcaccgcgcagatcctggacgggtttgccatccggcgacatgatgtagatggtgttaattgtctcaaaccaagatagttcatcaactccttcagtgtatagagttaccggaactgaagttttgatatgataaccatcgttatttggggtgccatcagacttcagcactggctccaggaaggcgaaacgaccatttggataaatcaccccgcctttaaacatccggatcttcttgcgagggttgttggcctcgccaaggcacacgaccagatgatggatcatctccggacgggtaagcaacgtgtcgtcacggatgataccctgtttaatagcggagatcaccacttcgtccaacagcgcctgctgctggtccagcgtcagtgattcagtaagtttcagagccattattcgtaccccaaacatttgtttaagaaagtcaggacatcatccatggtcttgacctcaatttcaatgccgagttgttccgccaacttgttggtgttctcgacgatctctttgtctcgcgcagacatgatgtcagcctttgaagttgatcggtttctgaaccccagcgtcaaacgcggcctgggtacgggtatcgctcaccacctggcaggtcttgtcaagatcgatgcgcgggtagccaaacacaccagcaatcaacgaaccagggaactgagccagcttggtttcgtacatcgcgcagatgtcaagcttcttggtctgactgatacggaactcatcgcggcctgagctgatgacaacctggatctctttgtacaggctggaatcgaactgaatgttctgttcctggatccactgcatcacagccttggaaccatcagcgccataacgcccttcgaatgtacctttgatcacttccttcaaaccgtctttgtacatgtcaggaatctgcgccgtctcctgaactttgagcgtgtagttgctcaggtagttctcggagtctttgttgaacttcttgacctgctgctcagtgcggttgaagtcattgaaatagctaaccacactgccaatcccaattacagcaaatgccagaatcgccaacccaacgatccaaccaattgaaatacctttacgttgtgacatctttacttcctcttgttaaaacaaacgtttataagttcgaccattgtactgcatgcggcctaatatgaaaactgcggtaaaagggatacatccaaacagcacgacgataatcacttcccaagttttaagatctcgccatttcaaatattccatttccttcatctcaactcggttgtatcccttactgatattggccgcaacttcttggaacacggatatgcccattggtttcccagtcaatgaaagtccgttacgagaatggagttccatgttgttcatgccgtcggcaaatgacgtagacttaccccaattaattttcatatcctttgaaatatcggtgactacaataacatcgttctttttaccgccagaccatgcatatagaagtccctgaaaatattcaacaggctggccagaagtcactacccacacgatattgacctgacgtgaagcacctaatgttttcagagtattgttcagataatcattccagtaatcaacaggaatgtccatccctgacatattcagaacacgagttactcggtaataatcatacaccctcggatatgaagggatatgctctttgaacttctcagcgaattgctggtcagatttagagaataatgaatctttgttgcccagcacataattcatatatgaatgttcacgtgccgcaggttccccgattttaacttgtgcccaacgcggaggctctgtagtaccctgacgatcaatacggtcaatgctcagatcaccgacggttgttaatacgtcccaatcaacgtcataagagtgctcatagcatgtgctgcaatgacgcgtttgtgtacaagatcggttatttcctgaacctgtacaagatgttgtgtaataacacatacattcataagaatgttcacaacccaccttattccgttgcttatcagttacatatccattcaggatttccacgtccccagaactaccaagggagaaagcagcatataatcccgactggataaggcaagacaatacgataacaatggccgtggcgatcaaagtctcctttggctcatgagtcttgcggtgatatatcacaaacaagatggccaaaattacaggaaccacaaacagaaggtaaatcataacacaccatccacgagttgttccttcgtcataccaagaagttgatttttatgctcagatttagccccatttttcaacaacagatcccaacgttcttcatgatactgtggagtcattgcatcgtcaaaagaaacatggtggcgatgcataattgttgcaacggtatgaagaacgattgctgtttctctttttaaagcacgtgtatccgtgatgtggtcttcgtattcgattcccatcaaatcgagttcttttcggatatatgctatagacaaatcaccttcagaatgcaatttaacaatagtcgctttcttagggtggagcgcaccatccgtttctttcatgaacagatcgtacaaatattgtttgtattcggggcgcttcatctcgtaatccctccggcacagatgatattcagttcaccgcgacggaaaccgccttgcccaccgttcaatttagtcggaagcgtatcttgaactttttgtgcgaactcttccgacactaccagatgacccttttcttgtttcggggtagattcaaaatagtcgccaaatgtcgcttccatatcgagcacagacatatcttcaattcgttgcattgccatgatataggacctcagtataatttcgtataaacttgtttaacgccaatatcggcaggagctgtgctctgctgcatattatagaatgccaaatctgcttgttctttgttttcaaaatcaactacaactgtgtgtactgatattgcttcaccctgtccataagcataacaacggctggtgaccaatattttgtattgaggtgtcataaatcaatccttgcaaaaagaaaggggaacgggctaataataacccattcccctttattgaagaacgcaatcgcaatcagattactttttcttcagctcgtccagtttggcttcgatttgttccaggatgtctttacccttctgaacaatcacttcaccgtcttgcgcgtgtttgcgaaagaccagagcgccagcgatgaagccgacaaccacaccaggaagcgcataaaccaacagatcttgtaacatgataatatcctcactttatagcgaggatatttatatcagcgtttcaagagtgtcatgaggttcacacttttagaaggatacacagtaccatttcggctcatggtgtccaatttaccaactgacaaacaaacctcagacggttcccaacgtgtaccctgctgtaaatgccagatatgtagaggaacacccgtctcaaagaacacgggcatcttccacttctggacgcctggaacgtgccctgcaggagctttttcgcacaacagggtatctggtagccagagtgaacgggcttcccagagaggtgcacaggagtattcaggccaaatacgttccatataatcgatctcgtcccatgtacgctcggcgtcccacccgatgtaacgggtattcttcagtcggaagaatttcttcagccaacaagcaccggaggtctcaaagttcagacgattgataaacgtgatgtggccaaactcttcaacgcattcctgaaacgctttctcaaggtcggtttcgagtatatcacattcttcgtctgtaataggacaaccgttaatctttttcttcccgtgcttggtcacccaatcatcacggttggataagaacgccgcgccgttacgattggattcgctaccatcacggtcacgcaacatgaacccagggacgtctatatcccatatgttaccgaagaccatgttcagggcttccaggaagttccaggcgctcaggcgaccgaagtatttccatgacatcgccgtattccacagttcggtgaactggtcttccttggtagtgcattccaacatccaacgaaaagaatccatttgggttttaccacccaaccaatcgatataggactgaacgcacggaatcattttcgacttacgataacgacaatctgtatcgaagcgcatacgatcaaagttcaggttataccaatcagaaaatcgctgaaactctttcggagattgtgggggtacaggaaattcactgtaaattgtccatggtccaatcccattatagcaacacccccacaaaaaggccatccaaatcttgcgctcgatcttttcgagctgtgtgatgccttctgtaacctcgatggcataatccatcagacggacttgctggttatgctcttcagtgtaggatagcgcctccacccatgccttgagcaggtacatgcgattttctggcttacgataatctactgccagatcaatcgggtatttccatttttcaggggctttatatccctgcggcattttagacagatgcgtcatccttcacctcggcttcgagttggatatcttcggggcttactacgggctgtcccagagaccagataggcaggaaacgacgatcattcgtcaccaaccaaatttgcttggcggttttcgtcttttcggaaataaaatgctgggcattgacctgggtacgagccaaacccacctgacgccaagaaccgaaatccttagaagaaggattggtattcagttgaaattgtgggacgattttattctgttccggataataacggacagcgttaatcatcttcgcatcaccacggtaatcaatagcaacagcgacaacgttgcgtggaatgaggattttcattttaattccttttgaaactctgaattatgttatagacgaaaggcgttaataagatagcaccccccaaataatataacgcgtaaacgttcaatatttcggttgaatccaactcaacgatgcccaaatatttcatgacaccgacaagcacagctgtgatacacatcaccaatccgacgaacacaaaatatttagacatctgtttcatttctaacccttactcaacaggccgagaagggatacagaggactttgttacctctcccttggcgctcgccttggttttagatttggtcgccttctcagggactcgatgcgctagaggatcatccccaccgacttcacgcgtcattgggtcagtttcacagaatccgtagaaatctttgatgtcgagatcgatcgcaccgatatccatctggtatgcgatggcatcaccaacaacccaaagaggagcatcaaacggcatgaccgctatttcgctgcggtggtccatgtcatacacattttcattaggcttcatatatacgatatcttgaccgctgtaagcatcttctaatacattccagcccagcgccagcatttcagctttcgacttctcaaactccttggagtaagattcgttgcgtgaccagccggagtcccctgcttccttaccagagcgaccacggatgcgtgcatcatactgatagcggtctgggtatgcaaaataaagcaacgccagagattcaatcggatagttcttgaacatccattcaggacgccatttatcagatagcatgaggggttcaccctcgcacaccaaagtgtaaccctgagccagccagcctttgaccagatcacgggcgatatcacccgagcctgtggcggcgtggatggcgtccatggaagtccaggaagccagaccggatttgttggacacggtatactggccgacgaagattaacttcagctcttcgaacttcaggccgaatgggcgcgtcttgtcaccaatggtgtaggtgagttcagtaggctccagcttagttcggagccattcgatgaactggaccactctcgtacccttgcccgtggccgaggtgcctttcactacaattattttagccattatgcatcttcctcaatacgccagctcacaccgcccatccatggtgcccagaacacagcaacctgcgggcggccattctcttcaaacttcactttatactggtcttcgaagttatagaaaatttcatcgtcatctggtgccataccgttttctatcagaacacgctttaactccagcgccgcaccaacggaagtgggtgtggtttgtttacgaatgaagttgttcacatggataataacgccatccatatcgctatcaccggaacgcttgtctggggtttccccaaacttggattgttcttgcacgatgtcctcctcaatggacgtattgataatggccaatcaggcggttcaccacttcgacaatattgcggttcaggaagtcgaagtggttctggccttctactggtttaatataaagcatatcttccgagttcgaaagatccagtttttcataatttccctgaggaacaaagatgacaaaaatattgttcatcgcacagggttcaacgtatttcgggttatcatcaattaaaacatcacctgccaaaagatgcttttcgtcggtgctgacaaacccgttgaagatgccagggaacttgtcatagacaaactggcgcttgctgcgctcgtgttctggctcacacttagatactgcgatcaattcaacattttcaaagtcttcgagaaggatcttcttcagattcaccaggaactcgtaagcgcctggaagcgggttcatcttggcatacaggtccggcatgcgccaccaatccataggatcacgtcctgtaggtgaatccatccattgaccagcaacaaacacacggcgtgtcaaccatgctgggtgagcgcgttcccgcatgaggatcgccaaatcaccagcatgagccatatagcactctttggtgatacgctggaaatcattgggataatcatggcaacccatgttttccgcagcagcttttgaattggaaatattaaaccaatccacccaaggggagaggctatcaaccaaagtcaagtctacgtctaccaggatacgatacattccaccgattttagaattggcatcatacagaccaacatgattattcatcagacttctccacttgatcataaccagttgcggtgcattgctcttctgtgcaatctgcatacagaaccacagaagattccttcacgtaagtggtacagtgataggtttcatcttcctggatggaatcatcataattgcaaacagtttgcaacggaccaggagtggtgttatccaacgcagcatgtgccttgatggtgtcttccatcttattcaccgcttgctgattggtgaacgtcggcgcatcgaggtcaacagtagacgcaacgtccagaagggtagagcatgcagtggtagagaaagccagagcacacaccagaagaatcttattgagtttcataacaatacctgttgtcaaagggataacagggggaattatatccccctttggtttattgaatcaacctttcttgcccatcatggacaacagattaactcgacgtttatgagaccaaccaaccacatccaagatagactgaattggctctataaaggtcttttcaaacgtggtgtggtaatccacccatttatccaatcccaattctggaggaaggaagtcagggaaggctatgcgatcattccccacaggattgccaggtttcaggttgatgattttaaccttatcaccggattcgatagggggaagaccgagatcttcgtgcttatcgatcaacttgttgtacataatacaagccttggcagcaaagtgcgtaccgctgatgtaattcccgttcgcgtctaaccacttctcaatatcgctcacaccagatgcctgagcgatatcatccacggtgagttccatatattcctttttgtatccagcgattaattcctgaacctctgcctcagtaccgagcaggactttctcataacatttaaccagacgctcacgacaccactcaggagtggttgatttacgcgcttccaaacctttgaatttgatctttggcttctcatacttgatgccttcgctatcgtatactgccatgcaatacatcttcttggctcgccatacagcagacgatgcgatgacctcacgttcccagaccatgcgctgctcaaacccgttcatggtattacacaacaactgcgcccattcactggtttttggctggtaattctctttgatccattggtcaatgttatcaacgagtttgtgatggtccttttcttcaggccacaattgcttgaccaggcgttcaatgcaaatgtagttggagtctgtatcacctgcgataacaaaatcctgaccagtggtgccacaaagtttgttcagataatcatcagtgtggcgtttgttccatttattgatcagctggccggaagtggtaattgcttcagcgatgttgatgttgaagtattctttaaaccagacgttagaaattgcaccatatcctgcgttcatcaagatcttcagaccttgttgtaacgtatcttgggcaactcccaaatcttcccacttgtgcatgacttcagtcaggtgatcaaggtcgatatgcttgtgttcttcataccattcaggatcgtagaaacgagacttcatggcagattcaaggtggaaatcacctttagacatttccttacaccaaccagcccattgctcatacttcaggccagttgctttctctcctttacggtcagcgtatatgcctcgcataatttcggaaaggaaagacatcttctcattactgaagaattgaacgttcggggtaaacgaaacgttatatcgacgtaatgtttcaaaatggaattcacccagcgcgaccaattcatcaacaacttgtatgcgttcatcaatagcacgctgcagcttgtcgtgaagatttttgagatgacggcgcttgttcataggcgttgtcatatcattcatcgctttggtcaattcttcacacatggactcaatgatatcgcgacgtgtgtgcttgtcagaaacgatagtctcaggaccaaggttgtattgctgtatgatgtggggatacagggagtttaagtcctcagagaatacccagaaatatatccctggtgcaacctccatgacatatgcgccttcaaagtccgttggaccatcatatacacgctgtattttagggacaatccccttttcataaagacgatagtaacacatcgccagccatggtgctacagtcccaagaccgtcttcatagttagatttggtgcgatacgccaatacgaacatcagctgtatcaaacgcagcttttgttctaaacgccacacgagtttgacgtctttgataccatatcgggtgtgtttgcaataatcattaaaatatagatcatacaatgacttgctttcactataatccaacttcttctcaccgagttcacaataagcgatccaatccagtgagtatttttcgcgggttgtgtacgtgtgtttcttgtaaacttgcatgtagtccatcataggacaacccacgaattgataagaagtcacgtcgcctttacggtctttgatgaaacgtttcttgagtttgccccaaggactgaggcgctctgcctgggtttcgccgagaacctgcgtaatacgttcaaccaagtacgggctatcaaacgtttcgatgttccagccagtccaaccatcaaattgacgttcagaccaataatctaagaaggcgcgaagaagttcttgttcagtcgtgtattctttgtattcaacctcaagaccacctatctcttcatcatttggatcatatttgaatttatggcgatccttagaacacggcataccccagacatagaacttgttggtgttcatgtcttgaagctggatgagcgtaatagggaaggcggcattcatgttctgtgtgatcttcccattactatcaatgatagggaactggtcagtcacgttgttggaaataaaggaacctgggaaatgctctcgaacgaaatcatggttggccaacacttgcttatggaatcggcgaacacgcgcctcgctccccttaaacgtgtgggattcaatcgtcgcgtgaggaaatgggcctttagtcatttctccatcgcgccacccagccgagaaaacttcgatatccacgttggcgatgtgaatatttgagtaatcgggggtaatcatcccaggaaaactatgcgctatgaattgatatgcataatccgtttgtccgtaaacggcagcgccttcgacctccttatattcctccagatagttgtcggcgtcacgcattgacgcaaattttttagacaccaacggttcattgaggaggccaatcttttcaactttggaataatcggctgtgggtaaatacaaggtgggttcgaatttcttacgcaacatacggcgattgccgttgtcgtctgcaatacgaatcagaaggtcgttaccctgtcgggcaacgttcgtatagaaaacagtcattgtttgtctccggttaaccgtgccgttagcacgtccccgtaatttagccgaggaacgttcaatgaccaaggagtttattatggcaatcccctttgtgagtatgatcccaggggcgctcaagcaactctggaacctcgggactgatttgatccaatacaagcgggaagttgttcaggccaaacacgatgtcaagctagaggctatcaagtcctcctcagattgggagctgtctaagatcaccgaagtcggcgggtcttggaaagacgaattctggactatcgtgttagctgttcccgctatcctcgtcatgactgctccggttgtggaactaatcctgttccctgatgagtatcataaaggggatttcatcaaagcagtaatcggcggcctccaggcgctcgataccgcacctgaatggtatacctattcgttattgacagcaattagtgcatcattcggtatcaaggggtataaccattacaaaagcaacagccggaaggcgcaagctgtggatgctctcaaacagtttggggtcaaagtggttaacaaagatcccactgttgttactcagtcgggttctcccgatcttgagtctgctcccccagctgggtctactgcgacctctggtgcgtggcctgatctgaagaaataagttagaggggaggaaactcccctttcttttggtgttaaggaatgaaagtggtaaacaagagattgctcgtcacccaaggcggggcgacgatagtaagaggaaggatttacgaacagattcagctgggtgatcataagacacgcctgactgtcaaagcaccggacggcggatggcccacaaaggtggttgtccccgacggcctcatagaacgagggaaagcagtgtacaaaggagcagaattcaaaatttattaaaatatttattgaataaagtttacttcttcaataactggcgtatacttctaattgtagtacgaacaaacaacaatctgaagagaaggaaatacatcatggcaatcgaaattatcgctcaatccaaatccggcaaaactgtattgttcggcaccgaagaaggccgtgtaatcaccactttgggcacagttgacggcagcaaccccgtaaaaggtggacgcttcctggttgagtacggttctcgtgtcgtgatggtaagtaaaattgatgggcgtgatatcaccgctcctcgtcaagaattctttgattcacgttctgccgccatgcaattctttttcttccagaaacgtcacgaactgttcgttgaagcgaaagacgcaggtctggacgacgcaacggctgagctgattgccgaaggtaaaatgactcttgagactgctctgggagacatggattacgaagccgaagcgcattatattgaatattccgcatatcagaacgaatgtgaagatgatccggaagatgatggatttgatccagatcttcacggcggcgaggatgacaatactccttctctggaagtgatcatggctggtggtcaggccgcttgggaatggcagaatgaaaagaacgcatggttggattcccgcctctaatatgacaaagccccttcgggggcttactttgaactgaaggactatattatgatttttatcgtgtgggtagtattcgcatttctggccatggccgacatcggcaagtatcatctaattcccagtgatctgactgtcctgctgtacggtatggtttcgtacatcctgggttatgtccgcgcccgtcttctgtctgaatgaaggactatatcatgagcattccaaaaactcctgtccgtattgccgtcgagcctctgctggttgatgctgagaatgctgctgagcagtacgcgaacaaagtcatcaagaacataatggacgatctggcatccaacggctgggacgtcgagaagtgcgctccgtggccatcttcggttggtgtcaagtttggtgatccagattaccaacgaatgaaatcaaaacatgcgctctaccgttctttgacccaggcagtgaatccttcttacatacctgggaagccggatattgtcaaagctgatcctgagcgccatgctctgtttgtcaaagaagcacgtcgaaacgctgcgatgcaatatgagtctttcatttgtaaactggaagacaaaattggtgggcacagcgcggctgtgctgaatggttcccatatatggggttattcaatcctaaccgtgactacaccagaggggatcgagcgctggaagactcagacgattatcaatgtgtccaagctgggcaaactctttaaccaatatccaacgaggaaggtgaaatgatgaaaaacttcgcacaagaattacagactacgattgaccaggctcagatttgggaagccatggggatagttgaaaaagccgttgatatctttgtttgcaaaatgaagaagcaactcgtttcccaagcaaatcgcggagttgaaattgatgacatggtgtacgctgttggtggccacaggtttgacgaaatattaaccgcatttcaacaggcatataagtctcaaaaatacccaacagacaagcaagtcgaaaagttcaaaacattgttggtggcgagtcttctgaaggaaggtttgaagttcgaattacacaagaatgaaagaacagatgtcgtaacaatgagattcacagtacacacaaacgatatctaaaacaaaggggcattaagcccctttgtttatttcccatcccaaaagtcgtcttcatcgtctacttcatcaaacgcagaatccaattcttcgtcagagtactctggaagacgaccattctgtttcagatattcaatttcgtcatcttccaattgaagatccgtggctataaacagcgcccctgtttcttcatcgtgctcaatactgtaagatgtatattcagcaccctggactgtaatgatgatagacgtcgggtcttctgggtccacatgggcttcaaacgggataccgtcaatcggggcatcctctaaaaatgtagcaagagctacggcggcgcggacgacgtctgtcccttgcacggaagcataaatctcttcagcgcctgtttccccgcgctcaaagaattcaatcgtagtcccgtcttgaacaccagtccagataggcatgtctgctggggaatccgcttcaaataatttcgaaattggcatggtaatgtcctcttgaataaaccgctttggactatttaggcttctctggtcgccaaccctggcttaaacaccaccatttcctcaacgttattggcactcatagaggctatggaatcgaaccctttgttgataagttctttcacattatcatgtatcttaccaggctttatccccaactcacgcattttattttcccacaccgaaacattgttggaatctattacaagagtcttcttggccttgacacgaaatgccatgacaataggttcaccagattgacgatagcgcacggcattttctgcgtagatctgtgccttttctggatcccccgtcaggtaaaacccagcacccatcgtaccagagtcagtctggccaaatttttcaaaatcaaagatatcgaattcaacattagacccatgatacaaaacgatattcgtaaaatcaggtaacgttgattcgcgataaaattctagaaaggttttcatagataccccacactggtcgtatggggtatttaggatggattagctggggacaaactcattcacagggacatgttcaggtattgataattcaggacaagcagctttcatcgcttccagatatggcttgctacccacgatccaaaacaatgagttctcattaagcaattctggtttgtgttcctgtatccatgtcatcaccttcccttcataacgagggtgcaactcaatatctccccactggtaatccatgaggtcgttgtaacgaacccaattagtggtatgaagatcccaatggtgtacctcaaaacgaggaagcacgatatccggctcgttctttggcctgataccacttaggaggccagccagcgaaacgctcttagaggcatgctggagatccttcttaccatacacataatctgggttgtcgtgatagcgacgagtgaaatcagttatgtgagggaggtttgcttgtttaccgataatgcgcaaacgactttcaatgaaatctaatcggtttgggccaatcccgatcagatagacattcttcagatttggtttcggatgcatcgccaaacctgtcaggatactggtgcatgaattacaagaaccagcaggaatgatcagatcagtgatatggtctgggatattggcaacctgctcaccacccagcatatggaatccagcaatgcgttctggtgaatgaacggtatgatccaatgtaatgccatattccagataatacgcctttggatttaattgttcaatgagtttcttacagcgcggctgaatggtgctattgtaaccagatccaacaaagttgaactcactaccaaaccatgctgacattgaaaccatatcatgattcatgcatgtggttggtttagtggcacccagcacagtggttgtcttgccgccgaaatgccgtgagactgccgtcgccataggggactgcggactaccaacgacagtaccatggataagatctggggatcctccagctttcagatgctcaaccattagccagatggcctgacggagtttgctgccattgatcccctgcttgccattcatatagcatgacaaaggcgcgaagtaatcttcacgtttgaaccaaacctgctgattggtttctggattggcaatgagttcgcacggggtgtgtttgtagagataatcttcccaatgaaccaaatcgcggttgaaggaaagggtatcgaagatggttttgttcattctatttctcctgtaggcattggcttatcatagcgtatctacaggagtatagaaaaggagaatctcagattacttagtcaggaagtccagcagacggcgaaagaacgatttgcgttctactggtttcttcgattccgtcacaggatattgaaggataggtgggagttcagggtatccatcagggaaacgtttatccagatcatttaaactacgactggccagaggagttggcttcaaagatttaattgcctgattcaaatcggaattatccacgacacccacttcttcaccgaccgtaaagcatggtttgattagaacggatttcttagactgctcatactgatgagcgatatccaggtatttcttgcgctctgcgtagaaatcaacattctttactttacggcggcgagcagtagaactctcttcaccatagaaatcataaccatttttgaataaccaattcacggcttgctcaaatgtattgaacaccatatacaaaccgcgtcgaccatttaccatgtcgtcttcgctggaataaccaatgaataccgcttgccctttcttgttgatatcaaaaggagtacacacaccagtttcttcaagtgatcgcggagcaagatgcccgtcccgatagatatatgccggatacaagaactcattcagagtacgtttgttccctttgtatgtgtacaggcgagcaaatggaggcaggtcacaaacgacatcgttgtaatcccaaatcgtaacattacgcatgttgtttcctcaaatcaatgaaggggtttataaacggttaaattttaaacccctcttcctcattgaacaaatcaaatactagacatatagagtgggacaacctgctcagcgatcaacacgacctcggtaatggtaaaatcaccaccataagagtacagtttcatttgcgctccgttggtcgccaggttcccatccttgtcaacgctgaagaatgtaatgaaagacaaagtgtccggcggtggtacgctagcatcacgacttcttgataactggttccccacagaacccacaaaatctaatatcatagttctattagaagtagacccagaccatgatccgacaatattaaccttcacaggaagaatagaatcatagggaaacgcatgaaatttgtgatctgataatttaaaaaacggggctaaaccagtaggagaagaagggtttggagtcttcccatctaacacggttaataaatctattccagttgctccagaagttagcagttgtgacaacccagaaaatctgacttctgttttaatgcggtgggtctgaagcatattccccaaatagggagatgtcaaaatagccatactccctccttaagaaacgataactgtcgcatcagaattaattgccttaacccaacccaccgtcggcggggttaaaacgaccatatttccttgatatatgatgtgcgcgttgttacccgttggaatactagggctatcgcacaaatacacagaaccgtgagtcacttgaagagttttcaaagatgttccgtctgagacttgtgtccatgtacttgcgtttggtatgatcttagcagttgccataatagacctcctttacaagtcagatctatttattatagacaaaaagaagcccgatcttcctgatcgggctatggttaaaccatcgtggggatggaaagactactcacagcaatttctggtattaggcgtttgggagagtgcgtcgggaatcgaacccgaaatcgtcaggatgctgaaagctatttgcatttcacttgcatatcctgcgactttcttaacttccttaaccccagattgttatgaagtcggcatgcacaccttcttcatcttcaggaagtcacactcattattctgattgcttcaactggattcgaaccagtgacctcaggcaccttgttctctcgaaactttcctgcgctctaccactggagctatgaagcaatttggcggaaggtgaaggattcgaaccttcgggggctaaaagccccactcgattagcagtcgagtcccttaaacctctcgggcaaccttccgtatactgcgcttcggtcaagaattttccaagccatgacctaaatctgggttcttgacctgaccgaaacgacttattggtgtgagtggatggatttgaaccaccgcgctgttacggggagatttacagtctcctgcattcgaccgctctgccacactcacgttgtttggtgctcgttagaggatttgaaccccagcactctggtgatcaacccagagagtcgcgcctcatccatttgttagcctaaacgagtcttgcaaaacccgcaccgaccgtctacaaacagactgcttgaccgagcgtaatatggacgggcttcacacccgattgtgcatttgatctgccgtagcccaactccccgcgcttatcagcgccgattggttaactggcaccagtgccttctggttcgatctcctggaactttacaggcttcgcgcccgaccatttctggaagttggggcatcgtttccgatgtggagtcaccccattttatacacagctggctgcaacgcttactgtgtttggtctaccaccacgtgctgggccacgcttgaggacatcggccatccttcctcagctaatccacgcggtggtagattttgtatcagagtgtccggtgtagctttccgtgaggtgcgcactactgtagcctctacactctgaatttggtgggcgttactttcgtcgggttcggattccgttcggctattggacgtcgttcgagagaacttgtcgttcgcgtacttcgtgcttacttgctttcgtttcacacccataattcataatttcctggataacattatcaaacaggaaatcctttaaccacttgatcccaaccacctgattatttttaggtggttccaccctcaaacgtaaatttttagggtattgcttcggatctaccaaaacttcaatcccagtttcaggatggtaaccataatagagatcaactttctcaccatggtatacggatttgcattttccgttgtagaaattagtcgtgcttgcgtcaagaaggtaggtattcttgtccatcatcaaggttttacattgcaccttgagcaaacgaccgttcacatcaatcacaaaatcatatggcgcattatctccgaaaggttgtgaaactgtgatcccgagttctgtacatttaaacaggaacctcgtttcacctatcatcccgcgctgttttgtgttcatgtgttcctgcaactattatttggcgacgatggcaggacttgaacctgcgacatacggtttaacagaccgccgttctaccgactgaactacatcgtctttatccgtgcttgttcgcttcgatcgcccttaattgggcgtccgcgtcagctttggtgtcatgcgtcccaagaactttagtcttggtgtggtcaaggacaacccacttatcgcctcgcttttcaacaaactcaacaatctctttgttgtcggtagcgagaccatcccactcagtaagaaaatcaccgaacgttttcatcatactcaccaattgtttattgaacaactggtttatttagtggtgaccctggctggacttgaaccagcgaccaatcgattatgagtcgactgctctaaccactgagctacggggccaaaatttggcggggtctgaggattttcacctctccttgctggtttggccgccagcctgttctctctggggactcgctgtccggacggctatcccttaaactatcgccccatattcaattgcaccagagcataattaccgctatacgaccaccctctcgggcaggttggttctgacccaacgtctctctgatgcgttttattgtgtgaccaaccaaggtctctcaaccctttatcccgtttgtgcatgatcagttcatgcatgcctgtttgtgcgcctcacggcaacgcgcttctgtgggtctttggcttgcaccgtagcctcaccacaggcgtttatttttccagggctttccacttgcaagtcttgaccatacagggatacgggcttgcaagatcgggttggacacaactttgatggctctggtactaaagatcccgcgtcacccatagacatggtttataaaatccagagccatcaaagttgcaaccaaatgttacaactcgcattcaatgtcttcgtcacacagaatacttgtcctctatcgctcggaccaaacgaggctggccgaagaaacaaaacaccactcaacactagagcgaggatgtggttgatagccacgatgtaatgtctctatcgaagagtgatcacacttgtcagatgatcactgttttgcgaactcgagaatactgcgcctgtctcgggtttccccttagttcggcctctcattgttcgtccggtcagggagctacccttgatgaccagcttatattgagatcttcgccagcgcccaccagtgtctagaatgtgtaggcgtcgaggtctgcaaggcattgccctatttcatacaacgctctctcaagtacgcaaatcgaagttctctgggcttaacaggttcgacccaacatgttgtatatcgcctaacttactcatgggacggccacttctgtgtacagaccttgagggttaaaagatatacccagagaacttcgatttgcgttgcccttcacttgccagagatcagggcaattggtaagacttcgctcacatcttagaggacactgcacgggactcgaacccgccacgagcatttggcgccaaatgcttccacctgtcaatgtcctctaagatgtggagccgttttagggagtcgaacccaaatgcttggccagcgctgaccttacatcccatctgacttaggcaacccacctctgcttactcaggagcttatcccctttggacagtcagatataacggcatcgtcgacgggagattattaatcacaccctcccgtctattttagtgcgccgaatttggtgatgaggaatcggaagacctcactggtgtatggctgttaagctactgccaggaacgtgttatcgtttgcatttatctttgtggtcagtttctaaaaacccgcaaagtcgcaagcaacgagaacgaaaacctgtttgagtcctaaatctacctgtcttggtctcacatcgctatgagtggccaggactcaaacagcttgccgttcagtgagttaactatactttgtaacccgttattgataaaaccttttatttttgttggagagggtgcggaatcgaaccaaacttctgagcgctaccccgtgtcaccaatgcaccatctcaccatcgatttgccggaacttacgcatccggcctcgcctgttggcgctcctaaaatccgggaagacagtaggaacaattatgttaccaacaggaaccttgttgccctttgacgcaacattatgaaccctctatgaagactcataatgttgtcggcgtgatggagattagcgcccccgacctccccttatatgttggtgtcgacttgccaacggggtgctctctgattctgagctaatcacgctacaacagggagtttcgttctccgacggtcaagtgcgtccactgacaccagcgaggtggatattgacttatggagccggagtcctcgtcctaggactgtgcttggcctcagcctttacaccgctggtcgcccgactctttacaccataaaatttactatactacagcccagctaatttgaaaagtcctctcccctttgagctacactgcccacagcagtgcggacgaaccttcactcaaaaccggatgcgccgaagtgggcaactgtttaccggactgatcgggtagacccaggagagagcaggtaatcccgatcaggaggatgaggagtaacatcatgtaaaacatagtgttccctcccttccatgcaactaactttactacaattatttattgaataaagcgttattttaacacttctaacttcacaacacgacgcagaaggtaaacatattcgccagcagcttcatgcgtcatataatgggtagaaggtgtggaatgattgttctgggtttcttttaatccaacacgttctgcccaaccattaatagagttcccatagaaaccgataatcgcgccaaaataaatgcgctggctctctggctctaaaacatagttgacagtgaagcaaatatcagaacgaatagactccagtttcataccaacacgaccttcggagaattcgttacgaacatataaatgacggaaacgatcgcgccgcgcttgtgatgattcttccaattcttgtcgaagtgaagaagttacgtcaatattaaatgaaaacgttggattctgtaaatcagaacgggccaactctaacgcagattcaacaacgcgacggaaattgatacttttacctgtcaggcgtttcacttcatctgtgattgttccaaaagggattttcgtggcgcgttccccttcgtattttgccaataaaggaataatgtgtttcatgattgctgtgttaatgtgcatttcggagcctcaccgaattgattaaatgtctccgttattatattggagatacgtttattgaatataaacacaagaatggccaataaaataatgacagtgattattaataggtaaaaactacttttctacaacatcaagaaactgtatcttaacgcggtgcccttcgagttgtgagtgttaacgaacaacgagaagcagcctccatggctgtggaatgggactgcctgtcaaggcggtgttacgagcgagggcgtcagccccctcgcgagtatattcgaataaattaaacgcgcgcgagcgttttgattcccgttggctaaatatcttcaacaccatattcaaactcaggtgatggagattatcatggccacgcaaacagttccttcactcgacgtcagagcatttgaatacattattaaacagcgaatgaaagctgatcccacctttaaagattatgactttgaaggttctggtcttagtgcgattattcgtttgttggcatctgacgcgaacgccatagcctttatgcaaaacatgcttaatggtgaaggccatttgaagacagcaaatcaacgttcaaacgtcggtttatctgctgcatttctttcctacacgcctgacaactatcgggcagcatacatgtatgtcaacatcaaagttacgccttatgacgccagcacagcacccaatgagatcatcatggatcggcgcgtaatgttcgttgggtcaaaagacggcagttcctacaactttactgttgagaaaccagtgtcagcaacgttgactgcagacggttattacatgttcaacaatgtaaagttggtacagggaaattggttgtacaagacatatgatgttgaaggaagcgcgatttcaacatacacgattccttctgggaatgtcgatatcaatcatatggtcgtgcaagtacaagaatcagagtcttccgacgtttcgacgacatatcaacgttacaacagtccatttgatttgagccaatatgcctacctgtatttcgtagaattgggtattgatggtctgtatgtgtttgaattcggagatggttatctttcgcgtcgagtagaagatgggaacgtcatattccttcaatacttggaaacttctggtgctgatgggaatgatatcacaagcctgtcatcagcgtcatccataggcgggtttaaccaggtcgatgtaaaattggtctccgaacgtagcgcaggtggggacgatccggaatctattgaggacactaaacggctggctcctttagcttatcaggctgatggtgctgcggttacagaaacagattatggtgtattgactgaaaggttattctccaacgtctcccgagctaaatcatatggtggtgacacgctgtctcctcctgattctggctatgtttacatcgctgttataccttctgttggtgagacactttcggacgctgagaaggccgatattgttgctgctctggacaagtataatgtgggatctattacccccaaagtggtagactccgagatcacatatatccaagtctcaacgactatattttgggatcctacttctacagtctacgttgaagaacagatgaaagttgttgttggaaacagtatcgttaaatggggtgaaaataatctcggcggatttgaccaattgtttgacaaagaaatattgcaggaagcaattacaaaaatggagcgttctatcaactcgaatattacttcagttggttataaacgccatttcaaacctgattatggagtcttagacagtttcacattcagttatggacgaagtatcaaacctggttctgtgaaaataacggggttcaaaccgcttcctgccgaggtggatttcacttactacatgcgcgacaataatggcgatctgaacatgtataaagtgaataacaatgacacgacaaaagagtttttagttcaaaaaacaggggttgtagattatgcaaatggggttgtagaccttcagcagatcacggtctctaactataaccctgaaggggtgaccatagtcgtgttaccggatggactgaaccagaacatacaggctacacaaaaccaagtattcaagattggtgatgtggttgttacgccagaggtgcgctatgtccaaagatcttaataatggacacaacggggttaagtatgaaactccgttgttctaccagaatgatttccctctgttcatagaatttatggacacgttcttcaattggctgtatagacagcaggggttcactcaagaagagatcttggcatatctggcagatacgtctagttggatgaatcccgagagtgaagattctccagttaaacagctgattgatttaaaggctgacaagaccccaggctcagaggccaaggattatttgtctgataaattcttggttcgtacctttgagaacatgatggctctggatgccgaagaacttttagatgccgacggaaggccgctactgtcaatagaagataagaacaaacagatagatgactggtataatgattttggtttccaacgaactgttgataaatcatttcaggaatttggtcattttatcccagttggttcggattctttattgacggccacgggcgatacgttctctgtttatatcgaaggaacgaaacggagaactctggatcatccgcgttggctcaaattgttaaaacatatctataaaatccgcggaaccaagaaagcaattgaattgttcttttggatatattttggttgtccagtcagcgtctattttacgaaagaagacataggcggtctggacggaaattttgaatgtgacggcacgacagggatgcgtgatgattactattatgatgaatacacttatgttataggagtcccaggcgacgtatctgattttgaaggtgtgtttgagcgcgtcttccgtcagcatttccatcctgctggatttactgtattcctagaaagcacaaggagctaaaaatggccgatttcctgagtcaatatacaggtcagcagatagatcagatattgggttctgttgatgacaaagtaagtaaaaatgacgttattaacgattttgaagttaacgacccattgtttccgccatctgctcgcctgacatacgagttgaaaagatctgttgacacaatcaatgatactttggccaacaaagttattcggacagatagtggtgaacaaacaatcaatggcaaaaagactttcaatgcgttgttggtgaccaatgggggcatacaagtccctgctggaaaatccgtttcaatcacagatgaaccaacgaactcgacagatggtgttaacctgagtatgttgagaaagcatggcgtcagtgaagacactaccccgcctggttctggtttggctggggagttgttgacatccggatattacaaattcaatttggatttcaccagattgtctgcgtataccggaccaacgttgccgacttcagttccagtacaaacagattctggagcgcattattggacaatgactgcattttttgatgcgttgtttgcagataaccgatttaatggatatgtgacaacgacagcgtatgacgcagatatgctgagtctagagtcccagattaataacatcaacacgtcgttgaacgggaaagtcaacacatcgacttacaacactaagatggcgtcgttggatttgagtatctccaatatcaatacgtctcttggtaataaagtcgaggtcagcacatataatacgaagatgacttccttggacggcagtatcagttctctcacgactggtaaggtcgatgtatcaacgtataatgccaaaatgaccacaattgataacagtttagggaacgcagtttatcgcacaaaaacaaacttgtcgtcatacacggtttcggcttctggaacgacggctcttccagattttgatttggtgaacctttgtcaaatactggtaactgcgcaatgggggacaggtaacgttgttgacacatatcgagtaaccataactcgtgacggaacgatcaaatcagagttgttggttcagaaaagtacgagcggtacagtgacgttcacaggggctgtggtgggtggtaaattgagaatttccgttgttaatgcaaatacgacaaccgcttgttctgtcgattattctattgcagcttctttctaaaactaaatacccctagatgacatgaatacatttaggggtattacaaatggccaacaaaccaacacagcctcttttccctttgggtttagaaacttctgagtcttcgaacataaaaggcttcaacaactccggcactattgagcattcccctggtgccgtaatgacatttcctgaagatactgaagttacaggtcttccatcttctgtgcgttacaatcctgatagtgatgaatttgaaggttattacgaaaacggtggttggttgtctttggggggtggtggaatacgctgggaaacgctccctcacgctccctctagcaatttgttagaaggtcgtggctatctcattaataataccacagggacatctacagtggttctcccttcccctacgcgtattggggattccgttactatttgtgatgcttatgggaaatttgccacttacccattgaccgtgtctccttctggaaataatttgtatggctctactgaagacatggctataacaactgataacgtatcagcaacgtttacttggtctggacctgaacaaggttgggttattacatccggcgtcggtcttggccaaggccgtgtctacagtcgtgaaatctttacacaaattttggcgtctgaaacaagtgctgtcactctcaatactccaccaacaatcgtggacgtgtatgctgacggaaaacgtcttgcggaatccaaatattcactagatgggaatgtaatcactttcagtccttctttgccagccagcacagaacttcaggtgattgaatatactcctattcaattgggcaatggcggtggttctggttcttctacgattacctgggtctataatgggggttcagcgattggcggtgaaaccgaaatcacgttagacgtcgttgttgatgatgttccggccattgatataaacggaagtcgccagtataaaaatctggggttcacattcgatccattaaccagtaaaatcactcttgcgcaagaactggatgcagaggatgaagttgttgtaattatcaatggaacgccaaacatctataatcaaattgattatactttgcgggaagtggctcgtgtaacaaatgttaaagatacagaggtcgtttattttagtgttggtgctgtgttaagcgggtataaagttatctatgataaagtaacacagagatcatattttattccagagctaccgactggaaccacggcagtcagccttagttcttcggctatacttgtgcattctgctggtagtgttgatctgggcgcattagctgtatctcgtgaagaatatgttacattgtctgggacatttgattctggtgctgtcatcaatactaaaaatgaattactcacccacacggatggtaagtatcgttgggatggggcattacctaaaactgtcgatgctggatcaacgcccgaaacatctgggggtattgggattggatcgtgggtgggtgttggtgatgcttctttaagatctgatttgaattcgacaaatggggctggaattgtaaaaactggaaatgggaatactgtacaagaaacatttgattttataaaaaatgttcaacaaacaaatctacatccttttgttccttcaggcaattttggttctggacttccttttgattcttcgttacaggtattcaagtataatgatgaatattactcatacaaaggacctattgtcccaggttcgatagttccggcaaccccatccgttgactcaaactggaaaaaagaacctgtttctgattacatagaaaattacggtggtggtgtgcatgtagctgatcagactcctgcaattatggcggctatatctgctggtgttcgccgtatcatatttcctaaaggtgaacttagaatgtctcagtgtgatattccagaatacacacaaggcctcacgtttgaagggcaaggtgccaatcaggcgtacaccggaacaacagtgatcaaaccgttaaatgataatcaaaattgtttatttaattctggcgtaaaccagcacggacaggattctataaaatttaaaaacattcaatttgacggagaatggcgttgcaaccatgcaatattgcatgagtctggagctggttggtactatgaaaaactgacaggtaaaaatttcaatacatgggttatctacgatgatcagggtttgactgtaatgcgtgacgtttataccgaagcaaccagttcagatctgacgaaaagagctacaggtggatgtatccgtatatactcagattctgtaatggataccgtaaactgttatggaggaagtgttccactatggattgctgctggcggaaaccgaattactaatatctgggcaaatggtgggtatgatgttggcgcaatacgtttgatgccaaagaacgacagtactaaccatatcaacactgccatgagcaatctctatatcggtgaaagtacaaacgaatcaggcactgctattcctgtattagaactacaaggtaactcaattcgtcgcgtatctggtgttcagatttcaaatgcacattttgtgcatgctgacttgactctcaatacaaacatggtgatgatacgaaccgataaagttgatggttttgtgatgtcatcttttgatgttcttggacgaggcgagtacagttctgcaagcagtcgcacatataatttcttaaatgcgacagattctaccggaatggctttttctgccggaagtgtaagaggtattaataataacccaatagttttatcatcagggtctaggttagctattgatgccgttgaattcgatgggtggagtacgaactctgattcaggtatagagaataaagctgctatacgttcatctggtgctggaacaaggtgctgtattggtagctcatgtgctttcaatgcttcgcaagcggatgcttacgctgtgtatgcaggggatgaatttgcagttgatatttctattcttaatcttaactatcctagtagcaatataataattcctgcaaatacaaattacagttacatgtataagcgaccaggcggaaaattaaccttagtcaacacattgacgatttgataacaagaacccatgattcataaattttattagttcagactaaataccccatattggtcatcctttatggggtattatcatgatttctcaattcaatcaaccacgcggctccacttctatagaagttaacaaacaatctatcgctcgaaatttcggtgtcaaagaagacgaagtcatttatttcactgctggtattgatctcagtggatttaaagtaatttatgatgagtctacccaacgggcttattctctccctttcggtattgtttcagggacaactgcaattagtctggatgaaagagctatcctcactcattccgctggctctgttgaccttggggaattagcagtaagccgcgaagaatatgtgactttacctggttcttttaatttcggccataccattaatgtgaaaaatgaacttattgttcacgatgataaaagatatcgatgggatggttctttgccaaaggtagttgacgctggttctactccagatagttcaggtggtgttggtttaggtgcatggttgagtgttggtgacgcagcatttagacaggaagccaacaaaaaattcaaatattcagtaaagttatctgattattctacattacaggatgcagcgacagcagccgtagatggattgcttattgatatcaattacaacttcacagatggtgagtctgtagattttggtggtaagattttaaccattaactgtaaggctaagtttattggagatggggctttaatctttaataatatggggccaggctcggtaattaatcaaccattcatggagagcaagactactccatgggtcattttcccgtgggatgctgatggtaaatggattacagatgctgcccttgttgctgcaacgctgaagcaatcaaagattgaaggctatcaacctggggtaaatgactgggttaaattccctggattagaggcattactcccacagaacgttaaagaccaacatattacagccactctagatattcgcagtgccagccgagtagaaataagaaatgctggtggtcttatggctgcttaccttttccgtagttgtcatcactgcaaggtaattgattcagatagcatcattggtggtaaagatggaatcattacctttgagaaccttagtggtgattggggattaggtaattatgttattggtggacgtgttcattatggttctggtagtggtgttcagttcctgagaaataatggtggtgaatcccacaatggtggagttattggtgttacatcatggcgagctggtgagtctggtttcaagacttatcagggttccgttggtggtggtactgcacgtaactataatctacagttcagggattctgttgcattgtctcctgtttgggatggttttgacttgggttctgacccaggtatggcaccagaaccggatagacctggggatttacctatatctgaatatccattccaccaactgcctaataaccatttggttgataatattcttgttatgaactcacttggtgttggtttaggtatggatggtcgtggtgggtatgtttctaacgttaccgtacaggattgtgctggtgcaggtatgcttgcacatacttacaaccgtgtattttctaacattacagttattgattgtaactaccttaattttgattctgaccaaattatcattataggtgattgtattgttaatgggattaaggctgctgggattaaaccacaaccatcaaatggtctggttatcagtgcaccaaactccacaataagtgggttggtcggtaatgttcctccagataaaattcttgttggtaacttacttgacccagtattaggtcagtctagagtcatcgggttcaatagtgatactgctgagttggctctacgtattaacaagctgtcagctactctggatagtggtgctttacgttcccatctgaacggttctgctggttctggttcggcatggactgagttaactgcactctctggttcaacccctaatgctgtatctctcaaggttaaccgaggagattataagacaactgagctacctatttctggtacagtattaccggatgaaggtgtgttggatattaatacaatgtccttgtacttagatgcgggtgcactgtgggctttgatacgactccctgatggaagtaaaacacgtatgaagttatctgtgtaataagtaacttatgccccgctttggcggggttcttattatacctttgttcataaatttattagttcagactaaataccccatattggcaaatcctttatggggtattttcaaatgactagaaatgtagaagaattatttggcggcgtaatcacagcacctcatcagatccctttcacgtataaatcaaatgttggtggagaaactttcctttccttgccgttctatcctgtcactggcgtagtcacaatcaacggtggtatgcaagttccgttagacaactttgaaatcgaaggaaatatgttgaatctcgggcgcgcattgtccaaaggcgatgttgtgtattgcttattcgataaaattctttcgccagaagatacagccaaaggtatccgcatatacaaatttcaggccgtaggaggtgaaaccgagttcactcctgatttcacatcttatggagtccaatctctttatatcggtggcgagtacaaaacacccgaaattgaatattcctatgacagcacgacaggaaaagtatctttgcaaactgcactgactgcaggtgtttgggtagtcgctgaaatgtctgttaaacaaccgaatatcagtccattgtttgaccgaagtattcaagaaatcgcccgttctgctaatgtaaaagactctgaagtcatcgttagtacggacaccatatctttgttggatgggaagaaagttgtttatgatatagcgacgcaaaccagttatggtttaccaaccattcctgatggttctgtcatttcttctgtatctggtgggaaattgaattacaacccaggagatgttcaggttaatttgttgccattgcctaatagtgctgcattactgagagaagaattaggtagtccaacgggtacaaatttggttaattttaagaattcggcagttggtgcattggtacgttctgttacttctaaattagaagataatccgataagtgttaaggattatggttgcaaaggagatggtgttaccgatgataccgctggattacaattagcagttgcaggggctttatcaggattggtcatatactttcctaaagggatttatcttaccagtttaccattgaattttaccaaacctgtgactttaataggagagaagggcgctcgtattgaattaactggtggtgatcatgattatgttatacaatttgattttcgtgatacagacctgatgtattggggctacgaagcatatatgagcaatattattgtagatggaaagggatttgctaaagacgggttttcacttcgaggagtaatttcttctagatttgataatttacgtgccacaaatatttctcgtgcggggcttcattgttggtggactcaatgtaatttgtataataattttcattgtagtaagaatgtggaattgtttactactacaccaaaatatgggatattaatcgattatgaagaagggtctccgaataaaagaggtacttctgccgatacgtttattaatcctgtttgtgaacatacgacagaagcaggtatcgaagcaacattctgtatcaatactatatttatgaatggtacttctgaaggttgtcgtttcggtatcgcctttggacatgaatctgatttggagcaaactgcggttacaaatacggttattggaatggacatggaagttaactctgatggcgatgtgttgttgcgtaaaaccgcgacaagtaacttatttttgggggttatggcgggttatagttctccatcgtcgcaaatacaaggcgctgaatttaaccgttggattggtggttcttcttctggattccatttttatggaggtacaaaggggaataaaatagaaggaactatgctattaggcgcgtcggccactattttagatgatgataaaacaaatgactgggattctttgtataacataagcaatgcgacttatacaggttcgactaaccctgttaacaaaagggttcaacatgatgggacagggacagtaaccatagatcccatgaaatgtagtttagcaacaattaacgcgacatcttccacaataaccatcaatactttagataatgccagagacgggatgcgtatggacatgattatctataatacatcagggcaaccacttactgtaaattggggagacagtttccgtattgggggatttgttcctccagaatctggtaaacaaaaaggattgtcctttgtgtgggatgctaactttgctcattggtattgtgttggtatgtctgcagatggtttagtttattaataatttacccctctccctctctcaggagagggtttattagaaaaacaggagattctcattatgaatccgcaatttgctcaaccaaaaggttctacttcaaaagaatcgaacaaagatagtattgcccgtaaatttgggtgtaaaaagtcggaagtcgtttatgccaaagcgggacagtcattgtctggttataaagtgatctacgataagttgtctcaacgtgcctacgctctcccttctaacattggtgctgtaacggtcaccagtttggtcgacggcatcctcacacactctggaggcaccgtagatttgggtgcattggctgtgttaagggaagagtatgttactcttgttgaaaattttacttctggatttacgattcgtgtcaagaacgaagtcgtgagcgatggggtgagtctatatcgttgggatggtgctttacctaaaaccgttgcacctggctcaacacctgaaacaactggtggagttggtttaggtgcatggttgagtgttggtgatacatcattgcgctctaatttaaaaagttctgatttaaatcttggagacgcgttagttacagtaaaaaaaccactacccctagccgttgcaaggtcgctacatagcgtaacgtcagagtatgttagtgtatttgattttcccggagtagtaggtgatggtgttaatgatgatactaacggaatcctcgccgcattatcatccagtaagcatgtgtatttccctaatccacctgcacattttgttatgaaggctcagatggtactggaagggcatactctgttagccgatggtattggtcgtatgtggggtaatacaggtgtagaaattcgtttttctggttttgatgatgattttgccattgtgtcaaaagataagacaaattttattaaaggttttctattcagaccagaggttcgtagtaatgccccagattatggtttacatattcaacgcgtaattcatatggcagatagtgctatagttggattcagtaaatctaacatcaaggggtgggaagatgtatcactaggatatgaaccttggggatgtacattccataattgcttcttcctgtatgctggagagcataatgtggtaatgctgcaaggttgtaacgcatatacttttgtagactgctcaacttcatgggctggtgctccagatataggcatcgcgcctacaactcctgctgagttttatgggtggtacgtatctcaagatgcttctttagcacctgttctttaccctacacagccagaaggtttatggattagtggtggagatagttcatacaacgctggtggttctgtttttgcggataaagggtttggtttagatgtgaatcttacttactcagagatgaataaaacatgctgcttaaaccttgggggaacgcttgccgctagccattttagaattggaagggcaactgaaggtcttgttacttctctacctgttgttacagacccaactacaccgttcccaggaacgtctatttatccggctagagatgtaaaccagatttatataaatggaagagattttggcacaggaatgaaagatagtgttaatggcaaattctataaatttgaaaatgaacccattactgttatgtcctccgctggtgcaccatacatagcaaaaatacctaatacctcaactggtggttttgaactaagggggtcagagcctgggttgcaaattaacctgaacgaagttaatttgatatcatcaacattccataatttaccagttaaattgaagaatcttgacccattaccggtcgctagcgccgcgtatgaagacgctatagtttctaatcacacggataaaaaggtgtacgtttgtatgtggaacgggacagcttatcagtggttcccaatgaactaaaaaaccccgcttcggcggggtttttatttctatctgtttgttggctgtatactaaatatctctataaaatggcactcgatggagcatcaaaatgcaatctacaaatttaaaccgtcgcccgtattgggatgactggaatcctgggaaacgtttttcacggattctttttcgtccgatgcctatcaaggttcagactcgcgaactgaaccagatgcagactattcttcaggatcaaattgaaaaactaggcaaccatttgttcaaagatggttctatggttatccctggcggtctgacgattaccaatgccgcagtctctttgaaattcactttggctggtggtactgaattcactgatctggaaggtatttctgaactctacgttttggggaaggacaataatgccaaagcgcgtgtgttgtctcttgaacgtgatctatctgctcctgatacgatgctggctatccttgagatgactgaagcgggtaatgctgacggattccatgttaacgataatctgtatttccagacttacgacgtgaatgataatttcattcgtattggttatggtatcgcggctgctgtgactggttctatcgttgctcgtatgacgaaaggcgtttacttcattcgtgggatgttcctagacgttgatgacgcaactcttatcgttgataaaacttcgaacacgtcttcccatcgcgtcggatttaaagtcactgaaaccatcgtgactgaatctgaagacgaatccctttattcaaatgctcagggaacgattaactccaaagctccaggcgctcatcgtcttcgaatagatctggtcttatctcgatttgattatgatgctgtggttgaagactttgttgaactggccaaagttcgtgatggtaaaatccagtctatggtgactcagtccacctataatattctggaagacacgttggcccaacggacgtatgaaaccaacggcgattacaacgtttcaacgcaccagatcgacatccgcgaacacctgaaagtcaataacaatggcggggtattcaatgctgctgatggcggtgacgaaagtaaattcgtgtctgtaatgaaaccaggtatttcctatgttcgcggtcgccgtatcgaaaacgtcggggaagagttggtgatcgttgataaggcgcgggataccgatgtcctgaataacacccctgttgcggtggccacaggcaattatctggtcaccaagaattctaaaggtgtgccagtgatatcccgcaccgttcgatacaaattactgaatgcttcaggcgttacccaggctacagcactttgtatatcagctgaacgtaattcaacagaatttcgtctgtatatgcgcgacctggtcgttactggcgatgcatccactatcaccaaagtgtcttacgaagaaagtggtatcaccatgttctctgctgaacttgaatccaatcagtttaaccagagttctatgatagatctgatcttctctcttccggtattcggtgtcaagactttagcaccaactggctccgtggatatcaactacactgttctcagaacgtataaagtgacgttggacaacagcggcgcgggttctatttctgctccattgggatatagtttttccccagaattttcgttgtactctgcagcgaaatcagatggttcggaagcgcagttcgatatctctggttctttgtctttgactggttctccggtcggttcggcgctgcagatttctctgggcagtggtaatgctaaccagtcaatcaatttgctggcgctgatgattcgtaccacggccacaatcaaaacgaagaccatcactgaaacaactgaaacagtgacgttcacctcgcagactagccgccctttggcgaatcacgatggttggaaattggtatcggtcaaaaacgatactggcgcagacgtgacctccagcttcgtcctagatggcggacagcgtgatgcagggtattacaagtctaacttgttatccagcgctggagcaatctcagggacgtatacagtggtgtatcaatacttcgcccacagttccggtgatttcttctctgctgattcgtatacttcgatggattataaagatatcccgaattatacatcttctacatcaggcgcggtgtacggcctggcagatagtttggatttccgtccgaaaatcaccaacggaacttctgacacagatatggttcgtccaaacactgcagttattctggacacagaatattatctgccgcgtattgacgcgatttatctggcagataacggtgtgtttggtgtggctcgtggcatcagttcgaacaatctggcttctccagcaatcccagcgaacgccatgcgtttgtatgaattgttgatcccaccgtatacgccgaatattgatgatatccaaattcgcactattgacaatcgtcgttatacgatgcgcgatatcggtaaactggaaacccgtatttccaatgttgaatactatacctctctgtcacagttggaatcttcagcgatgacacagcaagtgttcgaccccatcacaggcaatccccgtttcaaaaatggtattgcagctgatccgttcaaagacttccggttgattgatgatttgtctgaagattggatgggttctatcgataccgaaaacggacgtctgcgcccgtttgtacaacaaaacgttgttgacatgactcctgttggctggaacaaagtgatggatgggatggtggtttgtaattacacgcctgaaatctcagtgaaccaagaatatgcgacgacgacgatcaacgttaacccgtatgcagtattcaattgggaaggtttcttgaagattaacccgaccacagactactggttcgaaaactattatgttgcgccgcgtattatcaatgaaacgatcaacacccgtggtactgtacaagaaggttcagtgtacggaacatggcgtactgtttctgtttctgatcgtgtttgggaaccgcatggcgctggtggtgtgtggtggggatatcgttaccgcacgactgtttctgcccgtgatgtcaccacatatacatacacggacaaaacaaccacgaccatgactggtgaacagattgtggagacgcaagttatcccatacatgcgcgagatcgatatttctttcgacgcatctgggctgcgacctttcactcgtatgtatgcgttcttctctggtcgtgacgtcaatctgtattgcaaaccgaatggtggtaatttcggtgacccaatcaccactgatgccaatggtgctgttaaaggtatattccgcgttccacagaacgatacaatcaagttcaacacaggcgacaacgtgttccgcttaacggatagccctgttgacagtaaatctgcggatgatacactgaccaatgcagaaattgttcataaatctttcggtaagaaacaaggtattcaaaagacctttgtcaacactcgtgtcctgggttacactgccagtactcgcaccgaaaccagcacttctgaagttgtggtcgaccaatggcgtgatccgatcgcccaatcgtttatggtggcgactaagaatggcggcgagtatatcgaaggcgtggaagtattcttctctaccaagtcacgtgatgttccgatcactctggaaattcgagagatggagaacggcttaccttctcacacagtcattactcgtaagactttgaacccatctgaagtgacgatctctacagactcttccggcggtacgaagttcacctttgattatccggtgtatctgcaagcatcgactgagtttgctatcgttttgttggcgaatactcaggattacaacgcgtatatcgcggaaatgggcaagaaaaaccttctgtccaacgaatatatcgccaaacaaccgtatacaggggtgttcttcacttcttcaaacggttctacatggtctccgaaccaaatggccgatatgaaattccgcatatatcgttgcaactttgcggctggtcagaacgttgtaacatttgatccgaagctcgggccaaaacaacgtccgttgggattgaacactctgaactgcgtaagcgggtcttctgttgtgactgtgtttgcacctggtcatggtttggttgctgggaacaatgtcactctttctgaattgacaggcggttgtggctttactcctgaacaacttaacaaaacgtttactgtgacagatgcgagctatacttcattcaagattgacgtcggtacggcagcagacagcaatggacaaatcggtggagataatgcttctttcttgggcaattatctggttgatatgttctatgccagcgttaccaattcggctctggaaggttcaattctgaaattggaataccgttatcgcgatgccacttcaaattctatgtctgattgggctgagtttgaaactgacactgacgtggcgttaccgactgaaggtatctatcgtcaggttggtgacttccagattcgtgctactatgactcgtagtgaaaacaacgtgtacacagcaccgatgattgatggcgatgacctgagcgtgatcttcaactcttatggcgtagatccttttgaagacgttttcaaatatgtcacaaaggacattggtttcgataacccatgttcgactgtgaaactgttcttcggggcgatgctcccatctcagtcttcgatgaaggtgcaggtgaaacttctacgagcagggcaagagatggatagcgtggcttgggaagacgtcaccccaacttcgcctctggttaacgacggttccacattctttgaatatgagtatgacaagactgtggcgagcaataacccgtttgttggcctgaaagtccgagcgcttgtacgggggaaccgcgttgctcctccatcattcaaagacttccgtcttattgctctggcataaataatgttgaaaggtgggcgtaaagcccaccaaaccaatagaggataaagatatgcgtggagtgaaagtacaggggcacgcatccatgttgagaagcagttcttgccctggcgcgattatttgcaccgatcacgccgctggcatggctgcattgcaagcccgtcaacgcaatgaaacccgtgaagccaccattgttgaacaatcaaaccagatatctaatctggaggcgacaatcaaaatgatggccgaaaagttggggatcgaaatccccgaaggaggtcagaatggcgacgcaagctgaaagaacggggcttgacatccgtggtatcttaaccgctgtcgtggcttctgcattggtctctgcggcatcattcctttggttcatgggcgggatggaaacgcgcgtgaatgtattggaacgagattccaacaagatggatcaagttcttcagaaagtgaatgacatgagtgaacgtatggcgatcatgaatactgacctcgcatatgtcaaacaaaacatggctgagttaaaactcaatagcagcagtctttcagatgatgttcgcacactgcggattactgtatctgatttgcagcaaaaggggaataacaatggccgtcaataagattcgagacaaaaaatcttttatgaattatgtcttgcgtaaattgggtgctcctgtgatccaaattaaccttgacagttcacaagtcgaagatgcagtcgatgatgctctgcagaaattttgggaatatcatcgtgatggtagccaagatgcgttcttcctgtaccaagttaaacaagaagatatcgacaagggatatatagaatttcccgatgatattgacgatgtgattgaagttatccctggacctcctattgagtcaatcggaaactgggcaactcctcaatggcaaatggctcaagcaatgcttgtccccaaagcagcgctggtttctattcgtctcattgattatgtttccatgcaacaacgcctgtcggatatcacaagcgtcttaaatgttcgccgtaattttgtgtacaagaagttccagcgacgcctttacccgcagtttgctgccattgttgatgaaactctggcttttcgttgttatcaaaatatcgaccccgaatcggaagaaaacgctgaagcatggaatgatatgtggttgaaagcgtatgcgactgcattggtcaagcgccgttgggcagaggtgcttaagaaggcaagaggcatccgtctccctggtggtatcgaactggatggtgatagtatgttcagtgaggctgagaccgagatagagcggttggaggaagaattgcgtactggtcagcaataccctatcgattttatgatgggataagagaaggcgggttatcccgcctttctttttataacttgatgtcgcgtccggaaggatctgaatttgctagaatataaccatcaaccacgttcttttcgtcccaccattcaataaaatcattacccttttcgtctttgataagacccacaatgttatatgaaccatcggcgattttagcatctacacagataattctcacacgccttccgtcacgagtttctttaatatcttcaacattcatttatcaattcctcaaaagaattaatgattgactaaataccttcaaaattgagggtaaagtcatggctacttcaaaatatttcaactatacagcacaccaaggtactcaaaaattaattgatgacttggtggtcgagatgatacaattgcgtgggattgacgtcaagtatattccacgttctattgttgaaaaatacccaattttaaacgaggccgaacacaaattcgaccaagcgtttgacatcgaagtgtacatgcaggattatcaaggcttcaacacacagatgtgggaaaagttcggcggtatccaattacaagatgaagtgaccttcactattgctcgtcgtcgtttttcagaagttattggtaatggtccaggccttgaacaaatgcctcaagaaggtgatttgatatatctgcctatggctaacaaaatatttaaagtgaataacccgaacaatgatgaagaattcatgcaatttgggaaatggtacacatattccctaccatgtacgttgttccaatacggtaacgaagatttcgatacaggtgtatctgaaatagacgatatcgataaacgattgcaggatctggaaggagacggtgtgtacaaagacgccagcctgcaaacggataaccaatttgcggacgaaatagagaaagatctttcgcccaataaaatgaagatagacttcggggaataatcatggccagaccatttgaaaaatatttctatcatgaatcattgttgaaatacatacatgtgttcaacgctatcatgtcagatttaaaagtcaaaaccgaacgtggcttgatggaaattccgctgcatatggccattggccgccgtaatgacctcaacaggaacgtgccagccaatatgctaccatttgcaacaatgtcctttggtcagttcgaaatcaataaacaggtgacgaagtcataccacaaccaaatatcgaccgctacggcacggtccaaacaacgcattccgatcattatagattttgaatacaacatcagaactaaaaaattggtcgaaatgttgcaagtattagaacaaatttattctgtgttcactccttctgttgactgtcagataaaggacaacgatactttatctcaagatcagaacgtgaagataatgttggtaaatcacacgatttctgacaactgggaaggggacgcaacagaatcaccacacatagattgttcattcaattttcaattacatggtcacatctatggagaagattattgggttgatgatggttcgggcggtggggatccaaacgtcatcaaagagataattattgaaatgtctaatgatttgaacatgccatggtcggaacttcctgagtggttccgagttgataaagacggcatacatcatccggaggattgatcatgagcaatatgtcagaaaggttactcgccacgcttgatgcagtgaccgccagagacgaagtcgggaaggaggcgatggaggctgtcgcccctcgtccaggtattgacttcgatgaaaatacaggtgaatggtttggtgaacgtcctgaagggtatcagccgattcctgaacctccttcattggaggatattgcttccaaggaatccaaagtacctgagtttgcggacactgatgctacaacggattacaaacggatacgcgacacaacatatgccatgcaagaagccacaatgttcatgatgggacaagccgctaaattggctgcatccacagaagctccgcgagcattttctgttttccgtgaattgggtgaactcatgcgcggtctgaataaagacctgatggaaaaccagaaaaccatcaaggcagtaacaggtgataaagaaccacccgttgatgacacgacagtggatgttacaacttcaccagacggaacaacaacagtatcggttgggaaaaaggcgcgatcttctcgcgatttattgaagacgatcgaagatgcacgccgccgcgctgaagaaagagcacaagcaaaggccgcacaacaaccagaagatgaaatcattgatggtgaaacagttgatgtgaaggaagaagacg

SEQ ID NO: 2 - protein, 615 aa, Kuttervirus SPTD1
MNPQFAQPKGSTSKESNKDSIARKFGCKKSEVVYAKAGQSLSGYKVIYDKLSQRAYALPSNIGAVTVTSLVDGILTHSGGTVDLGALAVLREEYVTLVENFTSGFTIRVKNEVVSDGVSLYRWDGALPKTVAPGSTPETTGGVGLGAWLSVGDTSLRSNLKSSDLNLGDALVTVKKPLPLAVARSLHSVTSEYVSVFDFPGVVGDGVNDDTNGILAALSSSKHVYFPNPPAHFVMKAQMVLEGHTLLADGIGRMWGNTGVEIRFSGFDDDFAIVSKDKTNFIKGFLFRPEVRSNAPDYGLHIQRVIHMADSAIVGFSKSNIKGWEDVSLGYEPWGCTFHNCFFLYAGEHNVVMLQGCNAYTFVDCSTSWAGAPDIGIAPTTPAEFYGWYVSQDASLAPVLYPTQPEGLWISGGDSSYNAGGSVFADKGFGLDVNLTYSEMNKTCCLNLGGTLAASHFRIGRATEGLVTSLPVVTDPTTPFPGTSIYPARDVNQIYINGRDFGTGMKDSVNGKFYKFENEPITVMSSAGAPYIAKIPNTSTGGFELRGSEPGLQINLNEVNLISSTFHNLPVKLKNLDPLPVASAAYEDAIVSNHTDKKVYVCMWNGTAYQWFPMN

SEQ ID NO: 3 - protein, 729 aa, Kuttervirus SPTD1
MGYFQMTRNVEELFGGVITAPHQIPFTYKSNVGGETFLSLPFYPVTGVVTINGGMQVPLDNFEIEGNMLNLGRALSKGDVVYCLFDKILSPEDTAKGIRIYKFQAVGGETEFTPDFTSYGVQSLYIGGEYKTPEIEYSYDSTTGKVSLQTALTAGVWVVAEMSVKQPNISPLFDRSIQEIARSANVKDSEVIVSTDTISLLDGKKVVYDIATQTSYGLPTIPDGSVISSVSGGKLNYNPGDVQVNLLPLPNSAALLREELGSPTGTNLVNFKNSAVGALVRSVTSKLEDNPISVKDYGCKGDGVTDDTAGLQLAVAGALSGLVIYFPKGIYLTSLPLNFTKPVTLIGEKGARIELTGGDHDYVIQFDFRDTDLMYWGYEAYMSNIIVDGKGFAKDGFSLRGVISSRFDNLRATNISRAGLHCWWTQCNLYNNFHCSKNVELFTTTPKYGILIDYEEGSPNKRGTSADTFINPVCEHTTEAGIEATFCINTIFMNGTSEGCRFGIAFGHESDLEQTAVTNTVIGMDMEVNSDGDVLLRKTATSNLFLGVMAGYSSPSSQIQGAEFNRWIGGSSSGFHFYGGTKGNKIEGTMLLGASATILDDDKTNDWDSLYNISNATYTGSTNPVNKRVQHDGTGTVTIDPMKCSLATINATSSTITINTLDNARDGMRMDMIIYNTSGQPLTVNWGDSFRIGGFVPPESGKQKGLSFVWDANFAHWYCVGMSADGLVY

SEQ ID NO: 4 - protein, 708 aa, Kuttervirus SPTD1
MISQFNQPRGSTSIEVNKQSIARNFGVKEDEVIYFTAGIDLSGFKVIYDESTQRAYSLPFGIVSGTTAISLDERAILTHSAGSVDLGELAVSREEYVTLPGSFNFGHTINVKNELIVHDDKRYRWDGSLPKVVDAGSTPDSSGGVGLGAWLSVGDAAFRQEANKKFKYSVKLSDYSTLQDAATAAVDGLLIDINYNFTDGESVDFGGKILTINCKAKFIGDGALIFNNMGPGSVINQPFMESKTTPWVIFPWDADGKWITDAALVAATLKQSKIEGYQPGVNDWVKFPGLEALLPQNVKDQHITATLDIRSASRVEIRNAGGLMAAYLFRSCHHCKVIDSDSIIGGKDGIITFENLSGDWGLGNYVIGGRVHYGSGSGVQFLRNNGGESHNGGVIGVTSWRAGESGFKTYQGSVGGGTARNYNLQFRDSVALSPVWDGFDLGSDPGMAPEPDRPGDLPISEYPFHQLPNNHLVDNILVMNSLGVGLGMDGRGGYVSNVTVQDCAGAGMLAHTYNRVFSNITVIDCNYLNFDSDQIIIIGDCIVNGIKAAGIKPQPSNGLVISAPNSTISGLVGNVPPDKILVGNLLDPVLGQSRVIGFNSDTAELALRINKLSATLDSGALRSHLNGSAGSGSAWTELTALSGSTPNAVSLKVNRGDYKTTELPISGTVLPDEGVLDINTMSLYLDAGALWALIRLPDGSKTRMKLSV

SEQ ID NO: 5 - protein, 1013 aa, Kuttervirus SPTD1
MANKPTQPLFPLGLETSESSNIKGFNNSGTIEHSPGAVMTFPEDTEVTGLPSSVRYNPDSDEFEGYYENGGWLSLGGGGIRWETLPHAPSSNLLEGRGYLINNTTGTSTVVLPSPTRIGDSVTICDAYGKFATYPLTVSPSGNNLYGSTEDMAITTDNVSATFTWSGPEQGWVITSGVGLGQGRVYSREIFTQILASETSAVTLNTPPTIVDVYADGKRLAESKYSLDGNVITFSPSLPASTELQVIEYTPIQLGNGGGSGSSTITWVYNGGSAIGGETEITLDVVVDDVPAIDINGSRQYKNLGFTFDPLTSKITLAQELDAEDEVVVIINGTPNIYNQIDYTLREVARVTNVKDTEVVYFSVGAVLSGYKVIYDKVTQRSYFIPELPTGTTAVSLSSSAILVHSAGSVDLGALAVSREEYVTLSGTFDSGAVINTKNELLTHTDGKYRWDGALPKTVDAGSTPETSGGIGIGSWVGVGDASLRSDLNSTNGAGIVKTGNGNTVQETFDFIKNVQQTNLHPFVPSGNFGSGLPFDSSLQVFKYNDEYYSYKGPIVPGSIVPATPSVDSNWKKEPVSDYIENYGGGVHVADQTPAIMAAISAGVRRIIFPKGELRMSQCDIPEYTQGLTFEGQGANQAYTGTTVIKPLNDNQNCLFNSGVNQHGQDSIKFKNIQFDGEWRCNHAILHESGAGWYYEKLTGKNFNTWVIYDDQGLTVMRDVYTEATSSDLTKRATGGCIRIYSDSVMDTVNCYGGSVPLWIAAGGNRITNIWANGGYDVGAIRLMPKNDSTNHINTAMSNLYIGESTNESGTAIPVLELQGNSIRRVSGVQISNAHFVHADLTLNTNMVMIRTDKVDGFVMSSFDVLGRGEYSSASSRTYNFLNATDSTGMAFSAGSVRGINNNPIVLSSGSRLAIDAVEFDGWSTNSDSGIENKAAIRSSGAGTRCCIGSSCAFNASQADAYAVYAGDEFAVDISILNLNYPSSNIIIPANTNYSYMYKRPGGKLTLVNTLTI

SEQ ID NO: 6 - DNA, 158776 bp, synthetic construct
  source 1..158776: mol_type = other DNA
atggcgtatcagaagcgtgatatagaatatgccccaattaaaacggggttcaaaatagacgatgtgaaattgcgtatggaccaaacgttcatgcgcaaaccttctgtgcgtgctcctcgagtcgagttaatgctgacagatgaacaagaagacgaattcgttgaatgttctatggatgcacactacttcgccgccaactattacaagataaccacgatcgataagggctttatccttttcgatatgcatgattatcagaagcagttgttccacgactttcaggactatcgatttaatgcggtcgtccaggctcgtcagtccggaaaatgtgtaaaaggcgacacgcttgtcacagtttacgatactctcagccaagaggagttgctcctgactataggggagcttcacagtcgcttcgaggacgtcaaccacgctgtgccgctgaataccattggcaaccatgacaagttcgtggacagccgcttcggcaaacgttactttgtacaatccgatagtggctgggttccggttatcgcagcgcataagacgaagaaatatgcggaattcgttattgtcaccgttaccggaaggcgaattaacgtggccgatgaacatatgttcttcactccggacatgaaggagatatttgccaaagacctgaatgctgggtcatacatcatgacgacagaaggtccagaagaaatccgtgagatttggcagactggtgaatatcaccatatgtacgatcttcaggtcaaatctagtgatcaacgttattacacgaatggctttctcagtcataacacgaccgtggtggcggcgtttcttctttggtatgcgatgttccattctgataaggaaatcgcagtactggcaaacaaagagaaacaagcgatagaaattcttgaccgtatcagaaaagcataccaagaccttccattcttccttcagcagggttgtgagaagtttggttctaccctgatagagtttgagaatggttctaagatatacgcttatgccacgtcttcggactccatccgcggtcgttctgtatcgctcttgtacgttgacgaagtcgcgtttatcgaaaacgactttgaattttgggaatcaactttcccagccattgcatctgccgatacatcacgctgtattttgacaagtactccgaaaggccagcgagggttgttctatgatattgtcaccaaagcagatccgcgccatccacaatacaacgacttccacctaactgaagtcccatggtataaggttccggcatataccaaagatccagattgggaaaccaaacaacgtgcccgtctgggggatgctcgtttcgatcaagaatttggcattaagttccgtggttccgtgggttcattgattccggccaaatgcttagataaaatgacgtccaagttgtatcgggaacctaatgaattcaccaagatttataaggaatacgacccacaacgtctctactttgggattgcggacactgggaagggggtggaaggagattattccgtcttaacaattctggatataactgaatatccgcatgttatagcagccaagtacaggaataacacgatacctcctatgatgtatgcatacacgatagctgatatgtgcaccgaatacggggaatgccctgtccttgttgaaacaaacaacgacgtcggcggtcaggttattacaattctttatcaagagatagaatatccagaaatcatattcacatcaacagataacaaagggacggggaaacggattggtggacgtaaaccggaacctggtatcaataccaacagaaaagtccgatctatcggttgtgctaacctgaaagcgctcattgagaaagaaatgttggtgatagaagaccaggacacgatagatgaactcagtacatttgtgttcaaaggcgctcggtatgaagccgatgacggttgtcacgatgactgtgtcatgccgttggtcttatattcatgggcggtaaaacaagaatggttcagtgatttgacatctacaagtatttctcaggacatgagaaaccgaatgtcttcaactgaatctcagcaggtattcccatttggtggtttagtcgttggagacaccccatccgaaactgagcatttgcctggtttcgggggtattcaagttttcaacgaaatgtcagggatgaccatggatgagtggtttaagaattgatcactaaatatctttcaaattgagcgaaactctaccgagaaggaatgatattatggcaactcaaagcttcagcgttgcgccgtccgttcagtggactgagcgtgatgccacgcttcagacgtctccgtccgttgttgttcagggcgcgaccgtcggcaaatttcaatggggtgaagttgaacttccagtgctggtgactggtggtgagacaggtttggtgaagaaattcttcaaacccaacgacagtactgcgacagatttccttgtgattgcagactttttgtcttacagctctatggcatgggtgacccgtgttgttggtcctctggccaagaactctgttaccaaaggtcagacagcgattgcgatcaaaaacaaactggattttgaaacagcaagtccttcggcgtccatcacttggactggtcgttatccaggttccctgggtaatgatattgctatcaatgtttgtgatgctgctggattctcaacttgggaattccgtaataactttgcatacgcacctcagtctggtgaattccatgtagttgtcgtagacaaagttggtcgcattaccgactctgctggcgcggtcggtcaggttgaccgtatctccgtttctggtacggctactgctgctggcaccatcagtgtggcaggtgaagatatcgcatatctggatactgatactccagccgatttggcaaccaaaatcggtacagcactaacttccctgacaagtgtttattcttctgttgtcgtgaagtccaacactgtcatcgtgacccataaagctattggtccccagacagttactgctattgttccagatgataaggggctgactgcaaccgccgtgattaccactgttggcgcatctggttctattatcgaaaaatacgaactcatgcagaacacacagggttccaaaaagtccgatggcgcgaatgcgtacttcaaagatgtcatcaatgatacatcaaattgggtgtacaccttcgctaccgctctggccgcaggtgttgttgaattagaaggcggcgtagacgattataacatcaaccgcgtggcagctatccaagtcttgaacaatgctgaagcatatgatgcgaagccagtatttgcgtactgtgaagaactgattgagcaacaagcattgatcgacttatctactgagcgaaaagatacggtatctttcgtatccccgctccgtgatacggttgttggcaaccgtggtcgtgaaatggatgatgtcgttgcttggcgtgaaagccttgttcgcgactcttcttatttcttcatggatgataactgggcatacgtgtacgacaagtacaacgacaaaatgcgttggattccggcttgtggtggcaccgcaggtgtttgggcgcgaagcattgaaatcgcgggtatctacaaatctccggcgttccacaaccgtggtaaatacaacaactataatcgaatggcgtggtctgcatcttctgatgaacgtgctgtgttgtaccgcaaccagatcaacagcatcgtaaccttctccaatgaaggcatcgtcctgtatggtgacaaaactggcctgacccgcccgtcggcgtttgaccgcatcaacgttcgtggtctgttcatcatggctgagcagaacatcgccgcaatcgccaaatactatcttggtgagaacaatgacgcgtttacacgtggtctgttcagcaatgcggttcgtccatatattcgccagctggcaaatatgggcgcgatttacgatggtaaagtcaaatgtgatgaagacaacaacactgctgatatcattgcggcaaaccaaatggttgcaggcatttggttgaagcctgagtacagcataaattgggtgtatcttgactttgctgcagttcgaccagatatggagttcagcgaaatcgaatccggtggcggcatcgttgctgcttcataaaatgaccaaccccgcttcggcggggttttcctttgtcttattgatttgagttattgaatgatatacgcatacaaaattgaaacgataactaatggaaaggtctacatcggtgtgaccaatgacccacaacgaagatatgatcagcattttgcgaatgcttttcatcatggagtccaaagtgaattgtatgatgccctgcgtaagtatggaacatctggatttactttcgttgtaattgctcagaccgatgaagtccataaatgggaattagagaagcaactcatcgctcaatataattcttatgagatgggttacaacatgaataaaggcggtaatgatggaagtcatatgctgggtaaaactgcagcaaaattgacatctacaggtgaacacattggtcttgtttctctcatagatccccgttgggaattgtgtgaaatcgaacccgttttaagcaatgttgaccacggatttgatttttcttccatgaagattaaaagattgacgactaattagaattgtgtactcttatttggagatataaaaatggcgacagtcaatgagtttcgcgcagccatgtcacgagggggcggcgtacaacgccaacaccgctggcgtgtgactgtaaactttccttcttttgttgctggttccgacacaattcgtgacgtgtccttgctggctgtaaccaccaacaccccaacaggtcagctgggcgaaattctggtgccctggggtggtcgtgaacttccgttcccaggggatcgtcgtttcgaagcgcttcctgtgacgttcattaacgtggtgaacaacgctccatacaatgcttttgaagtgtggcagcaattcatcaacggtagtgaaaacaaccgcgccagcgcgaatccagatgattatttccgtgatatcatcatggaacttctggacgcgaatgacaacgtgactaagacatggactttacagggcggctggcctcaaaacctcggccaactggaactcgatatgtcggcaatggactcttatacacagttcactgtcgacttgcgttatttccaagccgtttctgacaagtcattgtaatgtcctcggtgctgggggaatgttattccccctcactaattacaagaaaatgttgaggacttagatcatggctggatacggcagagggttcttcggtttgtttggcggcggcggtttagtcaatgccaaagtcgacaccgataagttagcccaaaaacaagatgaattgttgctcaccaaggcgacagttgttgctctggacgatgctcaagacggttctataatccttcagggcggtgcaaacacctacaactatgttggcgttgagagtgaacttctcagcgttaaaacagttgtggaagaatatcagtccatggcccagcagccagaaatacgcaaagctgtggacatcattgttaatgacgttgtcacatgtgaggaagacgaaactccagtgacagtcaatcttgacaaagttgatggtatttctgaatcaatcaaagaatctatcaccgaatgtttcaaagaagtgattcatttgatggattttgaaaatacggcgtatcaaaagattcggaaatggtatgtagatggtcgacaagcgtatcatgtgattgttgacccaacgaataagaagggtgggatcaagaaattggtcatgctggattctcgttgtattcgtcctgtgtacatcgttgaaaaggcaatgcgtgaaggcggtattgaagcaatagaatctgtaacattgaagtattattacaacccgaattataatcgaaaccaattcactggccaatctggtacttcccagaacttccagccttcacagcaagaactcgtattcgatgacgaaagcattgtttacatcgatagtggtgaagagccattggccaatggtattgtcccaggccttttaaatcctgctatccgtccgttgaacaacctggttacgactgaagatgcgactgtaatttatgccatcactcgcgcccctgagaaacgcgcattctatcttgacgtcggtactctcggcaagaaatctgctgaagaatacatgaccatgatgatgggtaaattcaaaaaccgcaacgcatatgaccgcaccactggtaagataacaggtaatgcccatcttatgggtattgcagaggattattggttgccgcgccgagaaggtcagaatgctacagagattgcgactgttgggggtgggaatcaattgggcgaaatggatcacgtgaactatttccgtgaaaaactctatgatgctctaatgatccctaagagccgcctccaagaggaaggatctattaacattgggggttctaaccttgcggagattacacaggaagagctgcgcttcagcaagttctgtgctgggttacgacgccgttactcccatttctttatggaattcttgcgccgccagttaattttgaaaggcgttacggatgaaaaggattggaatgagaagatcaaaccgtttataaagtttgaattcacatccgatagttacattcgtgaacaacaagaaaacgctatcttgaatgatcgtctggcttcattgaacactgttgagccatttgttggttccattttctccatagactacgtcatgagaaatgtattgcgtatgtctgatgaagaagttaaagaacaacaaacgaagatcgcggaagagaagaagaaaggtctatatccgaaggttcaagcagatgaaactggcaattatagcggttcagatgttagtccgttgaagtttaaacctgagactattccattctccggttcaacagacgatagtatttaaagttcaactaaatatagaaaattaattcggagatcaaaatcatgagcgcaattgatatcgttcgtgcagtaattgacggtgacactgaaactgctgttgcagaatgcaacatggaacttgatgcacgtagtcaagaactattaaaccaaggcacagcatatgttctggattctatcgcagctgatatgaattcaaacaacaacggccagtaaggagatgcatgatggaaatcacagagatcgccacgttcgctgatttcctggcctctcgtatggacgagcagcgagtcatcgataaagtgaacgctcgtggtaaacgccgccgccgcttgaaatgtgcccctgggttcaaattatcggctgacggttcacgttgtgaagtcatggatgccagcgaacgtcgtgtgcgcaaaatcggcaaccgaaaagctcttcgctccaaaaagcgtatgggtatgggctaccaacgaaaaatcgagcgtcgtaagaaaaaggctatgaagttccgcaaaatgatgggactgagtaagaacaagtaaggagtttatgatgaaactgttgcgtgagatcacagcgatagggaaggatcttcaaatcggtgaggatacgacctcaactggtgggaaggccatgttcatcgaaggtccgtttgtgatgtgtaaccaagtaaaccggaacgggcgtaactatgatctacagaaagtgggtatccctgctgtcgaagcatatgacaaagagtatatccaagatcgtcgcgcaatcggtgaagtcacacatcctgactatcctttccctaatctggtggaagcagcgctcaaaaccgaatcccttcgctgggaaggcaccaatgccatcggtcgggcgcgaattttaaatacaccaaaaggccaaatcatacgtgcattggctgaagcagacttcaatctggccgtgtctacacgtggtctgggtgagaccaagtcggtaaacggttatgacgacgttcaacctggctttatgctcaccgctgttgatgcagtcgaccgtccttctggacaagtttgttatgttaaggctgtgagtgaatctgttgaatggcagcttgatgaggcttctggtatttggatgcctcgggatgttaaaggaaacgttgttgatcagttggtgaaagccaatattcaagtagaagacgacttcctgcgccgccttgatgcagcattgaatcatctgggctgaatttcagctcactaaatactgaaaaatcgttcagaaggaaactatcatgaaacctgaattgcaaaaactgtttgaaggcgttaatggcctcagcaccgatttcttggacaaagtatctggtctgctggaatctaaagttgaagccgcccgtctgcaagctattcaagaaactgaagcggctggtaacgttgagcgcctgactctggtagaagcccaccagaaagaagtcgcagaccttaaagaaaatttcactcttcagttggcggaaaaagttgattcgttcctcaacgcggttgttgaagaatgggctaacaaaaatgctccggctattgacgctcagatcaaaaccgaagctgctgaacgcttcctcactggtttctctaatgttctgaaagaagcaggtgttagtttcgccactgacccagacggtcagattgcagctctcaccaaccgcctggctgaagcagagcaacgcgccagcattgccaatactgaattggctcagctcaaagaaagtgaaaccaaacgccagcgcaatgatgtcattgatcgtatttgtgaaggtatggttgacaccaagaaagacactgttgtcaacctgctggaaggtattgaattccagaccgagtctgaatttgaatcccgtgtgcgcaccttccgtaacctggtagaaggcaaagatgacttctctgataaagtcggcaaagacaatgaaaaaggcaagccggatggcgacaagtctgaaaaagacatcaaagaaggtaaaaaaccgaagaaagaaggtgaagccgacgatgatgacgatgacgatgatgacaaagacgaagttggcaaagaagtcaacgaagccgtccgtcgccagatcagtgctttgctgaacggctaattttagcagccacagccctcgaaagggggcttgattttgaactactaagtaatttcaacttaactgaatatcaatgtaaggaacgagcatcatgactaagaaacttgtaaccgaagaaatgcgcaaacagtggctgccagttctccaaaaagaatctgaagctattcaacctctgtctgccgaaaacgtaaccatccgtctgatgcagaaccaggctgaatggaacgcaaaaaacctgggtgaatctgacgcacctggttctgtgaacagcactgtcggtaaatggcagccagttctgatcgacatggcaaaacgtctggcgccaatcaacatcgcgatggacttcttcggtgttcagccgctgtctggtcctgacggtcagatctttgcactgcgcgctcgccagggcgttggtgacagttccaacaccgcacagtctcgtaaagaactgttcatgcaggaagccgattccggctattccggtgatggtactgtacaggctggcgacccgtcaggctttagtcaggctgagatcgaaggttccggctctgtcgtgaccactatcggtaaaggtatgccgtcaaccgacgcggaactgctgggtactaccaccaatccgtgggcgcgtgttggtatcaccgttcagaaagcgaccgttactgccaagtctcgcggcctgtacgctgattacagccatgaactgcgtcaggacatgatggcaattcacggcgaagacgtggataatatcctgtctgacgtgatggtaactgaaattcaggcggaaatgaaccgtgaattcatccgtaccatgaacttcagtgctgttcgcttcaaaaaattcggcaccaacggtgttgttgatatcgcgcaggacatctctggtcgttgggcgctggaaaaatggaagttcctgactttcatgctggaagttgaagcgaacggtatcggtgttgacacccgtcgtggtaaaggcaaccgtgttctgtgttctccgaacgtggcatccgctctggcgatgtctggcatgctggactatgctccggttctgcaggaaaacactaaactggctgttgacccgactggccagaccttcgctggtgttctgtccaacggtatgcgcgtctatgttgacccgtatgctgtagcagaatatatcactctggcatacaaaggcgcaactgcgctggatgccggtatcttcttcgcgccgtatgtgccgctggaaatgtaccgcacccagggtgaaaccaccttcgctccgcgtatggcgttcaaaacccgttacggcatctgtgctaacccgttcgtacagattccggctaaccaagacccgcaggtttacgtgactgctgacggtattgctcaagacagcaacccgtatttccgcaaaggtctgatcaaatctctgttctaatataatccttgaattgccccggcctaaaaggaggtatattaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagcgacaggttttgataacaaaccccgcttcggcggggtttttctttatagggatatgtaagataataaagcctcatttatcaaaggaggttaaaatgtctcatcaattatctggcggtgcagtcgatactctattcgttcttttctggtttggacctcgtgaagctggggaaatacctgctaaatctggagaagccgaattggcctccctggggttttgtaaacgagttgatgttaaaaacgtaccaaaaggtcgagatacacatctgtgtgtactcaccgaggaaggttacaaatacgcatatctgaaatatgtttctcaaaatacgtgattgtttatcgtaacgatttaatacgcgaatagttcaataacaattcctgttgtacgatataactttcttacaacaggagtatcagtcatgacaacccgaaatattacctctgaacaatccgtgtcacaaatcctcattaattcccgcacccaaaaagaacgtctttttgacatcctcactaatgtgatcaatattcgtcgacaattagatggtgaatttcccgaatctgattctgaagaaaaatcaacacacaatccaggtcttgttggtgctatccttaacatacaaggagagcaggacgatatcatcgatagaatcaatgcccacatggacgtcatccatactaaactgggaacgttttatcacccaaattgtgaagacgagtctaaagataattgttaatgttttcaataaatgggtaatgaaccatcattacccaactttcacaataggaatacaactcatggcaaaagaaactcttaacaatgaaaactccaactttgcagaatggcataccgaattagagcgcgttgcccgttctcaacatggttctgccgctgatgcagatgcttggcgtgaagattatgaatctggaaaaactccacgtcaggcttgggaagacgaatgggggtgggatgatgccgactctaatccgtaagatatatctggataagaactatttggaatctaaaatcgaagttttcacggctcaaaaaatatttgaagacggtgaaacgagatattatctgaatccagacctttatctcccaggaagtaatttgaatcgcgacataacccaaatggacggcacgatcattcgttgggaaatagcgacgcaagaagacaactgtcacaacataacactttgaagagagaactacatcatgtctaaacaaaattatatctttattagtccaggacgttcccagttttttcgtagcgacatcgatacacctatcgaaggttttcgcattttcaaatgctataaattggccagcggtgacatcgattatcaaagtcacgacaatgaccgccaagacttggttgataacggttgggaagaaatagattctcctcaatatctgaccgtcaacctttattatatgaaggaaagcggtaaatattattcgactggggagttaaaaatccgagtttcagaagtccaatccaatccggcaaaaagttggtatgacacgcaagagttagtgcgtgatctcctgaagaaaggcgaactgcctggcttggttaaaggttcccgttttgataccttcatgtcctgtgaacaacacccgtgctatgttccgactttatttaaaatcagcaacatgtgagagattaatatatgagcaatttaccactgcttcaagtcggcgatgtgttcgttctcacagaagaaatgaccattgaaactcaggtgcctaagcatttcttgtattctaattgcaagggagattggcaaacggactctggttatatcacccctgagggattcttcggttacatgcaagggcattatgtcgttaccaaaacagctctacaaggaggtagcgctggtcacgacccatacccagacggtcatcatgtttgggcacagcataccgaaaatgaacgcatcaggatctgtttttatcagacaggcagcttcagaaacatgcaccgtaacgttccagtagtcggaaaagccaaggcgcaaacatggacttgggagaaattatgagaatcaaaaagattggagcaagccctttgacgggcactatcttccagggcactctgaataccaaaaccagtatgtgggtaggtgagaagaccgacgtgactaacgactgtgtagcggctacggctgaacacatgcgccacatcaagaaagactactgcttccctactaaagacggcaagttcctggtgctgagcgctgaggttcacgacactcttcctgatcgcttcaaataaatttaaaatttattcaataaacggggtttacttcttcaataaaccccgtatacttcattccataagcagcgcaacactgactctgaaatgaaggaactacatcatgacaaccgtaaaaatctccatctgggcattcaacaatttcaccaaattcgacgcttttgttaagaacgctctgggcatcaccaacatagaatctttcacttcttatgaccgcgaagatcaggtcaaaatgcttcgtcctgaaatcgaccttgacgcaatgctgaacgctatcaagaatcacgatgacaacctggctgaattcgaaaacctccgcacctctatcttcaaaaagaacggcggcattcgtaagctgagcaacaaaaccatcaaatatgtcaccgaagggttctttgtggtgttcgacttcatagaaaccaagccgaacgaacttcgtaaagatttagacggcaacgttatcaccctgaaaatgtttggtgaagcaaaggccgatatgatgcgtgacggcctgtgtgatgagacattcacatacgacgaatattgcgatcagtgccaagatctgtaatttgttctaaaccatttgaacgggtatgttaaaatcatacccaacccctacaaatagaggactataacatggcttgtaaaacaccagaaatgaaattagttgtttcttcaaaggggcgtcgcagccgcagtcgaaatctcatcacccgtttagatattctacgtcaaaaatatcaaggtcacagcgtgttcacacgcatccgtaacgcattaaaggaagggcgaaccgagttggagctatatcgtccaaacggttcgactcgtgcgtatcagaccaccgacggtttgctggaactgatccgcctctcgggaatgagcatagaacctcgttcatcagggactcccttgtgcagcctgtatgtaatcggcaatcttggggcgctgtgagttctatgaagaagtatatcgttcttataacgggttcccgttctataactgagcgggataaaatcttcgccaaactggatgaactgttagatccccacgagatcgaaacttttatagaaggtgaagcggttggtgtggacttgatatctcgtgattggtgtgaaatcaattatgtccatgttacgccaatggggatccctaaggattatcacacgatatatggggaaggcgcaggaaaccaacgtaacaaagatatgttagacaaagctacaaccctggccaaacaaaaaggtcttgaggtctttggaattgcgttatgggatggttcgtctactgggacgcaagacatgattacccgcatgaaaaaagcaggagtcaatgtcaatatcactcttatgggcaaacccaaaaccaaacgattgttataaatatgtttgccttcacacccatatggagttagctatgctgcaattaattttgattctttgcgtcgcttttaccatgtactttctggcgcgaatcagttatatcacactaaatgatctgaaaaatacaccacgcgaacctaaatttatatttctaaagaaatacaccaaaaagttctgcgagaattttatctatcatcttccatgattataaatacccccaaaattgggggtattttatgaacgcacaaatctacgatgtcctttcctctggtaacgcatttagcaatccgcttccgtccctctccggtccctcacaggatttaatcaccagtggtagtaccaatataccattgataactgccaacgcgactccggaaatgcagttggctatgtcagcaggtggattaacaccagataaacttacagcagccacaaacatgtatagctctgccaacacggggataacgacactgaacacatatggcgaccagagcataaatgaagcgtattcaagaattgggacttccgtttcatataaatcaggtttgaaaagtatatcacgggaaccaaacaattgtgacctgatcaacaaagcattcggtgttgttcaggatctaggccgccaatggttaaacgctatggaaggcgctctccagactgtgactaacaagatatcggaattgtatgacatgattatggaaggtgcctcagaaggtatggcaaaactacagcaattggctgctgaagtaacaggacatatcaatgaggcaataaccgccgtcaatggagttataaacgatatcaccgatgggatagctgcagaacttgctcatattgaaagtatgatcaaagaatgtctaaacttctcttttgcgaatgttcttggagaatgggccaaagatctctgtgctggaggcgttattgataaaatagggactccagaattaaaagagtccctgaaataatcaatgtctagcggggagaactcccactggttcgataccttccatcaccagggagttcaccccaacatcactcattcgaatccatactttaccgcctggtcttatttcatagatgttcatttcattacccacagcagatccattaacgaaaacaccctgagtggcaattcggatcggagatttggtgttattaactatctgaaatgatattgcaacgtctctggtgatttctacgtcccttgcttgagtattccttctgagtttgtcaaagaaagaagggttgaaagtcgctatgatgctattattctgagagtaataaatacttgcggatagatatgggatctttccttcgtcacctttcctcaataggggaatcgtggttttccagtatggtctcagtgactggttgttatcatcaaaacaaccaacagcttgaagagtctggaagttctgaaaatcagatactacttgattcagaatatttttattgcatttatcaatgtcggctgagatgttaacaggtatggttctgatttcaggatcccagaggtcggttacagaaatgacggacgtgacaaagatacggcaacttgtgagtgcaagcaccattatcatcgtcatgatccattttctgaatccgcgtgtcaggaacactaaaacgttctccacagcaaaggtgctaacatgacaggtgtacaattcaaaaacgaaaatcctaatttcgcggcttccgataaatggcgcttaactatcggagacctgatattgcttactcgttcaatacatgacttcagtattcctggagtgtattctgaaggtatcgagggtccatcccctggcgatgtgctcaactccataccttcagaacgtctcacgtatgaccccatcgtatttacgttcgttatcgatgagagttggtacaactgggaacagatttacaattggattgcgtctaatgctggatctgatttagctgttagccgcgacatcaccatcgaccttcttgataacatgaatcgacctattggtctacaattgttgttggaagaagcccgtcccacagctttggataacgtattggtggatgtggatgctgcagttccccaactggtcacgactgttacgttcaaatatttgaaactgactccgactcgcgtcacccgcgaaattgataaacaataagttataatacagagagttgaaacagcagggaataaatcatgagcgaaacaaaacttgaaacgatgaaaaccgaagatataatggcagaattggaacctcttatttcggtaaatcctgaagacatgaacttggaccaaatgtcattgaaaattggtcgttcgtggatgactgtgcaacgccattatattcgtgaaggaagatatctggagtatctaactggcaaattccgccaaatagatctctatttacgccgattttatgcaggggaattgccgcccaacgtttatgttgaacgcccattaaaagttcgacccctgaaatctgatatagacgtatgggtaaaagcagatgatgattatatcgaattgtctagcatgcttcaagagcagaaagccaaagtcaagttcatcgaaagttgcttagatcgtttgaacaaactcggatatgaagtgaagaatgctattgattggcgaaaatatttggatgggatgtaatggtttctctacaggctttgcgcaaacttaatgactttgaaaatcgtcatcatacatttcagcaatattccttgccgttcaaggaactgacttgcgtaactaccgaaaatggtcgtcactatgtttcccccactggagtcaaactgacttcagtgacgaccatgttagggcgtactggtgaccatacatggttagaagcctggcgggacaagttggggcatgaagctgctgatatagaaacacatcgttgcgcagaccgtggggaaaaacttcatttggcttgtgagttgtatctcaaaaacagaccaatgaaggaagttctggaagctgcaggggaatacgtgtttatgttcaaacaggttttcccctaccttaacaaaatgagcaagatatatgcacaagagattccattatatagcgaagtcctgggtcttgctggtcgtgttgacctcatcggggtttacgaaggaattccaagcataatcgatttcaaaacatccaacaccctaaaaactcgaagcatgatagaagattatagtattcaattgtgtctatactcagtcatgtttcaacaaatgtttaatgtcaagatagagcgtttaatcaatattatatccaacgaaaatgcccttgttcccaccataatcgaattcgatcgcaaagacgttatttctaaaatgtttgaccgtgtccgtctgtatcacaaaatggacaaagaacagaacggaatctgggcggatcgctaaatactccataatattgggggaaattagtgatgtctgagatccaaatagtcaaagtgaacgaagttagaatgcggatccttgccgaggattatatccgtgaagaactcaacgattacttcaaatttgaagatcctaatttccaaccgaatccgttcagtaaatgggacggcgtagtgcggttgttcacaaagagttctgggcttatcgacatcggcctgttatttgaggtgttcaaattctgtaagatgaacggttacaccatcaaattagatccggccttaaaatatattcaagatattccggatgaagaaattcatgcgttcatcgatacattgaatgcggtgtaccgagacgaaaacaaacaatatcaaaaagcgattgtccgcgattatcaatttgattccgttgccacagcaatacgccaaagcagatgtgtcctcgaagctgctacgagtgctggcaaatcctttattttgtacatcatggcgcgttactacaggcaacgcagagatgccctcgaaagtaatctgaggacgttaatcgttgtaccatccattcatttggtgactcagttgtatgacaacttcgaagaatatagccacgggacagaatggaagccattgatgaatgtccagacaatatacgaaggggcaacgaaagagattttcaaacccatagttatttcaacttggcaaggtatccagaaccaacccaaagagtggttccatcaatttggagatatcgtcgtcgacgaagtacatacatcaaaatcagataaattgtcttacatcttgaacaattgtattaacgctgatcaacgtttgggagtaaccggaaccctggcgaatacaaaggtcgcagggttacaagtcgttgctcactttggtgcatatcataaaattattacagcgcgcgatttaatcgatctaggatatgcggcagacatcaatgttaatatgattgaagtcatacatcctatggcagactgtattgaaatgcacggtgctgagtatcaaaaggaaatagaatttctcattgctaacgaacgccgcaatgaattgatagcccgaatggctctgtctttgaaagggaacgtcgcagtaatgttcgaacgtatcgatgcacacatgatgttggtttatgaaatgctatcggcagtcaagaaaaacgtattcgtcatcaacggggaagtcaagacagaagttcgtaaacagattcaggctgctatggaagaaggggaagatataacactgttggcatcatatggtactatgcaacagggcgtatctatcaaaaaactccatcatctgatattggcccatcccagcaaatcttatatccgtgttatacagacattggggcgtttgatgcgtcagcattcctctaaggatgttgcgcgaatctgggacctggtagacaacctgcgctacaacggttcctacaaccatgctctcaggcattctcatgagagatataaattttatcttatggagcgccaccctgttaaaatgaaaaaagtggcgctctaaccatttcaaagatcttgattcacataggcgggggttgcctcgccttctttgttggatccaaataaacagtcgatcgtgccaatacattgttgtagcactttcggctcaagaatctgagaaaacaattcttcattagatgccagcagtggaacaactttcacttcccgcgcctggccttgttgaataatgccaacatacagacctttgttatttgggagtaatggtatcaccaacatcggtgacacaataaaccccgatgtacagaaaaggctttgtaaataaacgccttttaatgacggacgttgttctgctatggaaagaattgcttcttggtcttcccctactgcgtataaaccaacaaattgcttcatatgtatttctcgcgtgttagcaaacttggctacataataatgtcatgctctgtttagaccaaataaaatattttaattgttgaataaaaatcgctttcttgttgaacaatccgtgcgtaatatacgagttgtgagctttgcgagcaacgagtattatcacgagcgaagcgagtagactgttaaagaaaggttttattaaaacagaatattataaatgccgtcaggcatttatttatcgcgcgtagggcaatttattacttgttctataactacaaacacgcgatactaatttgcgtcaaatatgacttgaaccccactatattttgaggatacaactaatgcaagagatcactctctccgaacgcactttacaactgctggctaactttgaaaccatctgcccctctatcgttctggcacctggtaaaaagttgcgcactgttaacgattccagtacagttatcgctattgccgacatcgacgaagacttcccgttcgaattcccgattctggatctgaccaaattgctggcaatccagcgtctgccgagttttaaaggcggtaaaattgaaatgtcagaagaccacattttgctgaaaggctgaaaactcccaactgcaattctggcgttccgctaaagaactgactgtcgttcctgcagacagcatcgatcttccgtctgtagaatttgaagcaacggtcactcctgaaaaaatgaaagagctgactcgcgcctgttctactctgggtcacaaaactgtacgtctggttgccagcggcggtaaaactcgtctggtgggcacgaccactactatcgataattccaacgattacactgttgagttgggtgaaacaactctgggtgattttgctatgcctgttgatgtggtcaacctgaaaatgatcgaaggcaactacgtgatccgcgcctgtgccgaaatgcaactggttaacttccaatccgcagacggcaccatcaactattacgtcggcatgcagctggattaatcaataacgattcggcgctttattatcggtggcgggggtttccccgccctttgaaacgttttatctaaagaggctgcaatgtctaacatcactatcgtcacagatcaatacgcgtgggaaaacaaatatcgtcctgacaatctggatgagatcgtcctcccttcagacgtccgtgcaaaaatccatggctatgttacatcaggcaatggaaacatcccctcaatgctgttctattcccctcagcctggtacgggcaagactacttctgctctggctgtgtgtaacgaaatcggttgcatcaaaccgttgatgatcaatgcgtcactggataacagcattgatgtcatccgtgaccgtgttattcaatacgcaactggcgtcagtgttttcggtggccgcaaagtcgttatcctcgatgaagttgagcgactttccatggcggcgcaggaatccctgaaaggcatcgtagagaaagtctccaaaaactgctccttcatcctgacgactaatgcgaaacagcgcgttaacgaacctctgcgttcccgctgtcgtgaaattgattttatctggtccgaaaaggaagcagatgaagttaaactccaattcatgcgtcgttgcgcaactatcctgacagaggaaggtattccgtatgaagctggtgtaattgcttccattgtgaaagcatatttcccagataatcgcaaaattatgggaactcttcaggataatgccaccacgtttggtaaaatcgatgaacgcgctctggcacaggttaaagcaggggaattccaagcgctggttgatttcctgaaaaccaatgattgggctgggatgaagcagtgggtgaccgacaaccaaaactacatcactgaagatttttattcccgtttcttccatttctgcgttccccagaacaaggaaaagactcctttgattcaaaaagagtcaatccctgatctggtatgtgtctgtggtcagtctcagattgaacatcgttccgtcggcgacgtctggttacacggcgtttacttcttgactaacgtcatgctcaacatcaaatggaagtaatattatggccgctccgtcactgttcgattacctcggtgcgttgaatagcaccaaagagaacctgttgatgtccgaagaccccgaaatcagaaaggcatttgatcctttcatgactcggcgcggccttgctcagagtaaagacacgcttgtggttgctgaacaaatgaaccgcttccatgccattactccttggatgcaatggaacctggcgtttcatagcattccggcaaaacgtcgctatggcaagtggtctaagaagggagctatggatcctgatgtaaaattgatttctgagtattattacatcagtcctgaaaaggcttcagaatatgttcgattcctgccgaaagaagtcctggctgaaatcaaggcgaaggtcgaacgttccaacagcaacgaaaaagccaaaccgcgcaaggctaagtaattcaaataattgttaaaggggtatgagtcatggcgcgtaatacgctggacatcttaaaactttcagctgtcaacgatgaggccagcattgttgactgtatggttgaagttcaattgaaccaagacaaaccagggattttcctgggtatcaaggaaacgctgagtcggataggggttaacactcgtcaagagcctaacaccttgtatcaaacgtgccatatcctgcataaatttggcagatatcacatcgtgcatttcaagcatttgttcatgctagatgggcactacaacggctttacgcgtgaagatgtcctgcgtatgaatcggattatacggcttctggagcaatggaatatggttaagatcttgcatccggaacaaataaccgaagtggctgacatgtctcggatcaaagttgtaaaacatgaccaagtttctgagtggaaactggttccaaaatacaccattcgcccaagtcgggcaaaaagcgaataactggagtcgctatattatgacaactacatttaataaaaacgtgagattgaatttggcttttggtaatgcggctggagatattacagcccctgacttcggtaaaattcgcaatcaggccaaactggttctggaagagacccgcgaattactggaagcggcttatttcgaccacgaggtgaaaattgagttcactgcatctccgcgagtcctgaaagaacctgctgttgtcgacatcaaagacattatgcgccagattctggacgctcagggcgacatcacaacagtcaatgacggcgtggcacacattgctggtttcaacggcgatgagtgcttgcagcgtgtttatgcctccaacatgagcaaatttattcgctcagaagatgaagtcggtccggcactggaatattattattcacgcggtttcccagatggccagttacgcgttgaaggtgaattccctcaggcatgcatcaaagtgaatgagactgtgatgtggaatggcaaagaatatccgaaaggaaaattcctgaaaaacatggctgtgttcaaagagcctgatttctctgatatgctgacgaataccccttcccgtcagatcaatgacatcgttgccagcatggcaattgaagaaggtcaggtatttgttgatgatctgaacggcgtggcgttggtatctccaaaaacgttggaagtaattctttctatggcagttgaaaaatcttctcgacccgtagatgttaacggccaaattgaaagttatgtgccagcgtatatcattaacgataccaatatgcctaagctgtgtggggtgtggggtgctaatgttcgcgtatacgcgtgcgcacctgttcaaaacactgtgacacacctctctcgcttcgattaatataatccgttattcactaaacaacagaggctataatggcctctgtttttgtattctagagaggataccatgaaagataaaccgtgtgacattccttattgcggtgttcgggaagctaaaattaaatctgcacatcccatgttaagcccattccataaacaactcgcatacgaatggatgagcgagcgttataagatccacgtgagaaaggacgtccagcgtcttcctgctccttggacggataatccaatactacggcaggtcaagttctgtaacgtccggagagagcacgacaggcagtctcttaacctgatcaataatatcgtcaagaacgacgctctgagcatggccgacaaaatgttcaactgtgttctgttccgcatgttcaatctctgggacccgatcaaggtggcgctcgagggcgctatgacgatttcagactttgctaaaatcaacctggacgagacccgcgcccgtcttcagaagtttgaatctgaaggtggtaagatcttcaccaatgcattcaacacaggcggtctaaaacaatgtctggcgttccccgaacttgttgtcaaccacaaggagcagcgcttcggtgggatgatggttaaggtacaccgtgttggccagggtgtcgtggatgaattggattacaaggtcgccaagaagctggttgaagagaatcctggggagtacacgattgagggttgggagccgtacatgccaatgcgtgtcatccgctctttgaaggcattcgtcaacaagtaccccgattacttcaatgatcttttacgtcttgactctccgctgaaagtttaccagcgcatgtatgacgacattgaagggcttgggccattcctggcatatcagatctgggtggacttcacatacatccctgagtatccattcagtgagaatcatttcactattgctggtccaggttgccgtgcgggtattgacctgatgttcctggataaggacggcatgactcatgaagaatgcatattctgggttcgtgatcatcagtatcaattgtttgctcagtatggttatgtccaagagcagttctggtcagccgaagcgccggaagatcagtgcatgaacgtcatgcagctggagaacatgttctgtgaattgagcaagtacacccgctgtgttgaggctatcgagagaggggaaaagccacgcggcaaggtcggttatgatggaggcgggagatacaaaaatcgatgcgatgaaattgtcctcaatcgcggatcggtcaatttattagatcgtttgaagaaacctgaataaagatttcttttacttcgttcttcaataactcttttcagcgtatattgacctcactttaacgacgcgccccgataagggcgctcactttgaactgaaacaggaagactataccatggcagaattcaaacccttcgcaacagcagttaataacaacgtgctggcgatgtccgcaactggtctgttcatgaccaatatcgataaagatgctctttgggatctgtatctggcatctttccctgctggtactaacccgctgtaccgcgaacgtactgaacacgattgtacctgctgtaaacaattcattcgtaacatcggcggtgttgtcaccatcgattcagaactgaacgtaatttctatctgggacaacattgatctgggtaatgaatatgacgtggtggctgcggcgctgtcggcgtatgtcaaacaacacgcaatcgtcgatgtttattttaacgataccaagaaagtcggtgttgagtcgaatcacgaaatgaaagacggaaatgtccgcacctacaaccactttcacaccgacctgctgggtagctatgttctgcgtccggacgcaatcgcttcgaaaaaaggtgaaatccgccaggctattgaattgtttgaacgcggtctgcgtgaaattactctggaatcagctgaaatcgttctggaactgattgaccagaattctctgtatcgcggcgcagaacacaaagcggcagtgaagggatttgttgaaaccaaaaaggcatatgaacaaatccccgaatcagcccgttctctgtgggcatggcatactggataccgttcaaacaatcatgttccacacggcatccgtaacacagttatcggcactctgctgacagatatcagcgaaggcgtggaactggaaaaggctgttaaatcgtttgaatccaaagttgctccggcgaactacaaacgtccgacagcgctggtttcaaaatccatgatcgaaaacgctcagaaagaagttattgctctgggtttggaagactcactggcgcgtcgttatgcggtttatgatgatctgaccatcaacaacgttctgtttgccgacctggctgctcagaaagcgatggatccgtttgctcagctggccgccgaagtgaaaactccgaccaagtccctggaaaaagttgaagagatcagcgttgatgatttcctgagcaatgttctgccaaaagcgcattcaatggaagtgcttgtagagaattctcatactagcaacctgatgtccctggttgctccggcgaacccagatgcgcctaacctgttcaagtggaacaacggtttctcctggtcttacaatggtgaagtgaccgattccattaaagaacgtgtaaaggctgctggcggtaaagttgacgggtatctgcgcgtttccttggcgtggcacaataacgacgacctcgatctgcatatgttcaacaacatggagcacgtttacttccgcgatcgccgctctatgacaggtgcccatctggatatcgatatgaacggtatggatggtattgatccaaaccgcgctccggtggaaaatatcatctttacagatgaaagcaagctgcgcgatggtgaataccgtttcgaaatccacaactacaatcaacgtgaaaaaatcgacgaagggtttgaagtcgaagtagaatacaaaggcgcaacgcatcgtttcagccatcctggcctgaaaaatcaggggcgtatcactgcagtcatcctgactgttaaagacaaacaagttgttggcattaagtccgaactggctaatgttgataagtctaaagaagtttggggtatcaagactgcgaccttccagaaagtacagctggtcctgaattcaccaaaccattgggatggggagcaaactggtaacaagcacgtgttcttcattcttgaaggctgtgctaatcctgaaggtactcgtggtttctacaacgaatatctgaagccagaactggataaacatcgtaaggtttttgaaatgttgggtagtaaaatgaaagtccagccgaatgaaaaccaactgtccggtctgggattctcgacgacgcaacgcaaccacgtgttcgtcaaagtgtccggcgcgtttaaccgtactgtaaaagtcattttctaattgattttgaccacaaattcgtaataaggaacaatgaaatgtttgatcaagcaacccgtttaaaactgcgtttcaactctaacaaaggtctgctgtctgttgagcaggtttgggatctgaacctgaatgccctaaacgaactggcgaaagacctcagccgtcaggtgaaggaagccgccagcgatgaagaagatttcatcggcgtgaaaagcgcagtcgactctcagttacaactgcgtttcgatatcgtgaaagcgatcatcggtgttaaactgaaagaacgtgacgaaagcgcaaccgccgccgaacgtaaagcgaacaaccaggttattctggaactgatccagcgtaagaaacagcaggagcttgaaggtaaatctcttgaagaactggaagcgctgttgaaataatctgtcggctccggcctgagcaaccccgccgtgtgcggggtttcttttaggtaaagaatatgaaacaagttatttgtgattattgcggtcttcctgccaaatacgtcagtggtgatgtactctaccctcatcgcccagatctacacaaactgaagttctgggtatgctcgccatgcgacgctcgtgtgggttgccatacccatggggatggtagacaacctctgggaaggatggcgaatgccgctctccgtgctgctaagcaggaagctcataggtcgtttgaccctatctggaaaagtgggcagaggagcaggagtgatgcttacagctggctggctcataatctggggatcaagaaacgcgactgccatatcggtttattcgatatcgacacgtgccgtaaagtaatatccgtgtgtaaccaactagaggtgtcaaacgatgtcaaaggaaatacaagaatcttttgaaaagttatctcaggtaatgaccgactgcaaccgccgaaattattgcgatgaagcgtatacgcaattgaaaaccgtcgtcgaatctgagttgaataacctttcaacttttaaagatattcagaccaacgtgctgtatttgtctgatgaagatctagaacattgcccccgtaatatccgacaattactagaccgtttagtgaaagtgggggtggataatgggtaatcgtgaagggattaaactcactccagaacttctaaagtcccacatcgccgaagtgatttatgaagaccgtgaagttggtggtcatcgtgtcattacatgccatttcaagatggacaatggattcgtcgtctgggggaagaatagttcaacttctatcgacccagccaactttgatgaagagttgggtaagaaactggcctatgaaaagaccttctcccagctgtgggaactggaagcatatcgcgcgatggtagaaaagcagatcatcgatgtgtccgtcaaagatgctaaaatcaacattgatgaatatgtgtcagaaagggaagcgatacatgagatgggtggttctgaattccttaagggtaaaggacaatccgtcgtgtttcgtcatcccaaggatccaagtcggggatttcgagttgaatttgttcgcaccgataatgaacgttataacgaacgacgtaatggtgaagcaagatctttggtggaagcgtatcttcgctcattcatgacttctgaagaactaaattctgaacttgtcttgcgtttggcgaaaacgtgtaatgctgcttcaggggattatccgtggtgtgacctgtcagaagacaaacgccaagcagcttgtgatcgtgttaagaaattcttgttagatccgaaagcccaacctggtgattccgtgggggcgctgtatagagccatcgtggtgtcctacttgtgattttcaaaagtctttcaaaatcaataacgtccggcaaattgctgggcgtttcaattaacaagccaaggtataatggtctaattgtaaagcgaggaaatcatgtccgtaaaaatgaaaattgatatgactaaaccatttttgcgttcaggagttgactctcttgactcacagtttaaacccagcgaagttttaattcaacagattgagactaatcgcgaattcatagaacacatgctggcgaagccggaaggtgaagccgtccataccactaaagaaggtaataacgatgattgaatcattaacatttaagaatgcggttgcgaccgtcgcgccgtctaaccatgtaatcgttgtggatatctccggttccatgtatcgttcacttccagaagtccgtaagcatctgaaggagaacctgccttccctggtgaaaccggaagatactgtgagcatcctttacttttcatctcgcggtgatttcggaacggtatttgccggacgtcagattaacagtgcaactgatctcagtgagatcaataatctaattgaccgtttcctgaagccttctggttgtactggttttgttgagccgctgaaattggctgcggaaactgccattagcctgaataaacctggttatgttaataacctggcattcatgaccgacggatatgacaactgctggcgttctaatgaaatcctggatgccgctgaagtactgcccaaagcgtttgataacattaccttcatcgaatacggttggtattgtaatcgcgaactgctggggctaatggcagaacgttctggtgcgacccacgtattcgccgaaggacagaatgaatatcagaccgaattggaaaacgtgctgaaaacttcttcaccgaaagttgtagtcgatgttcctctggtttatacccatgtcatttatgttgaaaatggcgtggcgactgttttagctgtgcatcctgatgaagagcaccctattggccacgtcagcatccctgaaagcgtttctcagttgtgggttgtaaaccccaacatgatcgaccagctggataatctggcagacatccaagcggcatacgtgctggcattttacggcgtgtataccatggacgcagatttggtttgggctgcgctgaagaaaacgggtgacgtccgcttcatcaaacaatatagcaactgtttcaccaagcaggactattccaacatcaaagtcgatctgacccaggctatcgtagacgaatctctgcgaggagttgatggcatcgattataatatggttccggctgaagacgccaccaccattgttgatgttctgacgtatctggctgaagcagatgtctctatcgtcaccaagcatccgctattctcttataagagcattgggcgcggtactgtacagaaagttgatgacaccgaagacaagttggccgaagagattgcgaatgcgacttctaaagaagagcgtaaagccctggcgctcaaactggctgagcacgaagattggacccccgaattcaatccagcagatgataaagggattgtcccgatcagcaatttggtctataattcagaacgtcctaacatcagtgtgcagacagttcaacacggaacggttgttgtgcctgagtttgtacagaagaaatatgaacttcctgaagagctgggaacctggcgttatcgcaattacaccattgtcaaggatggtatcatcaacctgaaaacaatgcctatcactgcagattctccgattgttcgggctaaggtggcacaggatttgactgccatgggtgttcatgtgtttggtgggccggaagtcttcattgtaaatctggaaagtgttccaatggttaaccgcgccatgacaaagaatatcagtgctgctgaattctttgcagacaatgtgcgccgtgaagcattgaaggcaaaacagaaagttttgaaattctaccgtgatgaactggttggtaaaggcaacgctactggtctggcttcaaaatatggcaaagaagcggctgatttcctgtcagcaaacggcattcgcgactatggattttcaccgaagacttcttctgtagaatcaaccgatgtctatatgagtcgtgaattgaatgtgaaaatcaaaggcgcatcttcactgccgagcatcgcgtctgttatcaaaaaacagacggacggtaaaaaactcaatgtcgctgatcagctgatcgctaatgccctggctgaatacaatgcgtttgttaaatcgccgatgatcaccagcgtaccggaagagactcagaaaaagctgatcgaaagttggattggtgatgcagcaaaagcagctatcaacgaagttcgctctctgaacaaaacgctgtccaaggtagtttatgggattgttgcaggccatggttggtttaccgacctggatttggaagaatccactatggatgtggaagttgatggggtgaaatacaccgtcactgccgaactggctgaaaaagaagtcaaaatctaaattcaactggcggggaaacccgccttcattttgagatatgcttatgtcaaacaaaccaacttatgttgaagtaatgcgtgttctggctcagtacgcttgtgacaacctgaacatggaaacttggcgcaacggggatcacaaggacaagtttgataacattgatgataatatcgatgctttagcgccgttacgggttaagaatgatctcggcggtgacgatcttgacatgattgagctggtcatggaaatcgaagaagagtacgatgtagaaattagtgacgagtgggtgggtaaacatggagatgatcctaccctcggagagttagctgaactcgtcgttgccctgcgcaagtaactgaagatccccaacaaacgttggggatttttacttttaggggtttacttctataaggcaaatgcgtattattgaattcatcgggcggggcgttcccgttcattgaaagaaggtatatattatgtctgctgtatcacgcgttgttgacctcaccccattttgcctgaacaatattgctaaccctaaattggtattgtttaaagatccctctaatttctgctggctggcaagttgtgatacccacaacggacgtcgtgctgtttgggagtttcccgcaactgatgaaggtagaatttccgctataagaaaattagaactggaaaattcaagccgagaaattaaacaggcataacttttattaaatcccaacaaacgttggggatttttacttttaggggtttacttctataaggcaaatgcgtattattcaataactggcgaacgatgggttcaccaggttgaaaggtaagattatatcatgaagaacaaaatggtagaaacttcagtggagattgatcgtaattggggcttgctccgcgctaaagggttccgtcgtgtgtatgcgaccaaagaagtatatgcccagcgccgcgatgttatcatcagcaagaagggaaaagattgggttgccgaagaagtcgatgtcgaaacaggcgtcgtcggaaaggtgaaagccaaggccgcgatcctcttcgctcttctgaagcacatcagttaattctataacttagggtggggtatgattaccccgcctatttgaaatgcaggaactataccatgaccgatcaacaaattcaagatttattttacctcgtccatgataagtcccatattatgcgccctggcactgtttctcctgtcgtccgtgatctggtgcaagaattacgcgaaccgcagatgaccacactttatcgtgggttgcctaaagaggacgttgtgcgtttagagatgatgtttgaacgaggcgacatgttccagacatgggattcttgtcttagcttcagtgaattgaagcatgtggccaaaaaattttctaaatcgtatggaaccagagctgttgctcgcatttgttacaaacatcgaaacggactcccagaggggcttaatgtctttggttctctagaaaaatttgtgaacaaagacgatgaatcagaaatggatatgctggacttggcgtccatggagaaagaatggatattctgccagcctcgcgtcttcccagtttcaagaaaaatggcaacagatgggacgatggtttttaccgtatcacttattactctataaacccacggcggggtatctttgccccgcctatttgaaatgcaggaactaatatcatgacagatcaaagaagtattgctcgttcggaaatcaaagatattatacagagcaaaaccaacgaaactcatgctcaatccagaatgctatctcgatttgttgaaattaagtttgacattgaagagaataacaatcggagagcgctggagggaatgttggcgattatcttgggccatgtcaccaattcagcatacgacatggatgatttaactcgtttgttgaacgcccagaataacgttgggacttctgttcgcgaattgctgtttaagtccgaatgaactaactaagggataaccctatatcccgaggtgtgtcatgttccttacatcctttattgactggatgaatatcataaatggtctgaggatccctgttgggaacctcgggctttctcgttcttctatgccgcaaatcgatgccgacaaacaagaagattttcacaaatatcttgaagacaacggcgtgtccatagatgctgcgcaagtacccatcaaaatgcttcgtcttacccagaacgaaatcaataaaatgaaggtctggaagattatgaagcagatcagaaataagaagcccatgggtcgagtttgggtgtcttctgacaattacgttgttgacgggtctcaccgatttgtcgctgcgttgaacatggacggcaaacagcgcatgaaggtctacaaagttgatttgccagcgatggaatttgttaaactcgctaaacagtttcagggtgtgcgatacagaacggtcagcgatacccgtttctgaatacgatagtttattgaaaccctttactcaacatggtgacatccctatcattttatccaatgaaataaggagaatcaccatgaaacgtttaatcatcggggttgtgctggctggtctggcacttcctgccaccgccaagctgaactccacaggtgaaaaactgtggtctgatctttcctactgtgctgggttctcccaagctgttgctatagacaaaactggttccatagagaactttgctgaactctgggccacaggtaacgtcacagatgccgttgtaaacgctggtattgagtttaatcgatataagcaaggcgcgttcaacctgaaaggatatatgaacgacgaagcatttaaccgaggaggcatggaggcgagcgatcttattatgactggtcgtatggacacacaaggacgaatgaccgtaagacagtgccgttctctcccttctccagcattacaaaaaggtgtgcaggttggccgacgtcagctggacctcatcaatgatggaaaccaatgtatccgagtatttgaatatagcgctgatcaagaatctgatcgaagactgaaaaaggaatggcggactcgcgcccttggcctgagagcatggctggtggaaaatgatcattattatgaagaccgtgttcagaatggtttgaaagatctttccactaatttgtctgtcaatcttgatgatcccaggcttgcccgtgaactcaagcaaacaagaattgactgtgataatatgatgaaggagaacgaaagatgaaaatcaaaatcttattgttattcctgtacactttcatgtcttcccaagtattggcagctccggccagtaagttgaatatttgtgctgcttggttgtctacatatcaacaattccatccggatgataaggatacatcaatccttttatctgattatgaaggtgaattgaaacgccttgatctctatagtgtggaccagatagagaatgcgcttgatttacccatgatggaatctgctgcagacaacagcaaacagactattaaagatttgaccatgtgccgtgaaattgcaactaatttcgttggaaatcaaagatagtttcaggttaaaattttattccccattgaactgaaaggtgtatttcgtgatgatcattaccaaaaagatcgctgaggcgatcgttgaaaagaataaaatgttcgcacctagcaaagcaaaggttattctgatccatgatgatggtcgcgtggtcttcgctgaaaaatttgagcaagggtgcgctttctggtttgcagacactatgcgtgaaagcaagctacagggcatcatttacaaattaactctggttgccaagcgcaacgatcagggtgactgggttacggtttgtgatttcagctccaaggttgatactcgaccaaaatctcgccaagagagaattctggaagctcaggaaaaacttgttgctgctcgtacttcaccagtcattaagccagcagtcatctccccttctattgatctgccgcctgtcagccaaaaggctgttgagcaatatgcagaaaaacgaaaagaagaacccgtgaagtctccagttgagaataaagaagaatctgaatttatttcaatcaggcagttatgtccgcaagaaagacgattttcttcaccaaatgatttattcgtgtctttccggtctaatgggaagattatattgtgcaaaaccctacgggaacaattgcagtggactaacatcaatatgatggtgagtcggaacttcaaacgattcgcaatcggtgaaggtaaagattatcctgttaataagagcggaacatatgcaaatcgtcatatgtgcgacaaattgtctttcccaaacgattccggaaccatccgcgttcgtttagaatgggatgaaagtttgaacatgtatgtcggtgatataaaataattcaataacattagggcggttattattaaaccgccttttatttgagagatctatattatgaacaacaagagccgtattatcatcggggctgaccagggtaatttatattttgaaagatctggtgttgtcgcccacgctatccttcccactggaatttccgtttattgtgtggaacatcctgaccctgcagttaaacgtctttgctatgtctcccgtgaaaatggactaattgtattgccagaaggcgcgattatgcgcgctcgtttttggccgatcgattttaattccatgagcaatagggagatcgacgcccgtttcttcgcatataatatgaaccggactctgaaaaatgaaactcagagagttgttcatttcgacaatgaaactcatggagtgacgttagaattcagaactcgtggtgaagctcttcaggtgaagaaatcaagctggaaaactgaagggacgttcacatatgattatgcgttaatcgacaaattgtgtaatcagtttcgcattagcctgactgccaacgacgatttaggtcagtatgttgtatggacagcccgttcaccacatatgaacggtatcatgatggagagtgaaactcgtaatcgcgctgtgctccaactgctgctgttagacgcgtattctcgcaacccgttggaatatctgtctcgggaacagattgataacgcgctttctcattgtaaccgtctggaggtgtaatatgtccggcgtatggtgtgatgtattgttggtaagattttcaaacgggatggataatcagatcatttcattcttttatcctgttcccttttcttctatgggtctgactgtcaagcgcgttcttgacatagaagaaaaactcaacacgatgccgtatatggcgcgaccacacgtgctgggttgtgaactgttaaacgaaatgatgattgacgagaatcttgttgaaacaattatcacgcgtgaacagatgaaaaccatactgtctcagggagaaccccatgtcgaataaaccatttgaacttaaatttcgcgtttggcatattccccaggttcctgggatcgcttttcatgttgaggttccaacatttgaagaagcgcagcgcctgaagaatactctggctgattacgacctgtttcagtataacaataacatcaagccggattattgtaatgcttccggcgttcaaatataccaacatgatctgactgaagaagatctaaatgatatgggactggatgatcgttgggttgatatcgaagatattgacgaactcaacgattattttgatcatctgcgtagcacagggtgggaaatatgatcgggttaatccgttatctgttcatattcgtaataacaacattcattttatttttctttttcaaatacttgaaagagactaaaatctaccggagtaaacgttggcgcaaacgggtgagagtaactttcctactgctgatttcttcggttacaataactgcactcacattgggtgcttatatttacatttttgaaggagtacattgatgttcaagaaactggtatttggcgcaattatggtcctagcggcaagccttctttcaggctgcggcggtgttatcgatgaaggaaacgtcggtgttcgaacccaatggggtgaagtggacatgaaccctgtaaccgcaggtatctacaccagtttcgtttcaagtgtagatgtctatacgactaaagaagccgtcgtgagtctgacgaaaatgacgccaaaggccaaagacaacctgaccttggaagatctggatgtggacgtttattacacaccgaacgtcgcgaaggttccttggttccataccaaatttgcaggtcagagcgctgaactcggtgacggcacgatagcggtgggattcaacctggttaaaaccgccgcagcatcctcaacgatggacgccgtttcttctttggattctatgacaattcacacccaacgggctgagttagaaaagatgatcgaggaacgcacccagcagcaattggaaactgctgcgcctggcatgttcacaatcacacgtgtgttggtgaaaaaggcgctgacagatccttcgattgagcaatctatccgtgataatgtcatggccgacaaacgtttggatacagcaagaaagaacgttgaaattcgcgaccaagaagccaaggcgaatcagaaactgacgacatctctgacacctgaatatttacaacacgaatataacatggtgttgcaatcttgcgccaacagtgggaaatgtactctgatcgttgacggatccggctcaggtaaaatgctgaacgtcggcaaataaacgctttattcaataatatttgttaggtaagctgtagtcctcttgtagtgaaacatcgtaaacaaccaactggagtaatccgaatgaaccgtactgattttatcgctcacatcgcccagactcagggtatgaccaaaggcgaagctgagaaaatcgttgcatcttttatcaatggcgtgaaaactgcagtcgcggctggccaatctgtccaattcgtaggcttcggcgcgttcgaagttaaacatcaggaagcgcgcactggccgcaacccgctgaacggacaacagatccagatcgaagcgaaaaatgtagttaaattcaaagtcggtgaaggtctgaaagctgctgccaacggccagtaatcacatccagagcggtcgcgcctagcacagcgtgttaggctaaaagagaaagccgaggcattaacctcggctttttcgttgttgggattcgggtaaactataacagaatccaaaggaggaacaacgtgacagcatttaatcaacaccgaaaggcgattgacgccataggccgtaaggttcctatggaagaaatacaacgccttgccaaagatgaattcccatatgacaaagcgaacccaggccagatggaatgcatcgtcgaagccatagatgcgcttatcaacaaaaaggttaaacatgtcatcattgaagctccgacaggtgtcggtaaatctttgattggcactaccattcataaagtgattcgtcatctggttttacaggctgatccttatgggcaatttcgcacttctatcagcaccccgaccaaaggtctgcaagatcagtatgctgctgaaaaagcagtatcgatcgatatcctaaaaggcaagaagaattatcgttgccacgtacatccagatgtgtattacaatgctgtccaatgccgtatagcatgccgcgacgggcattgtagcaaacgccgttgtccttatgtacaggcgcgtaatttgtggacggatatttcatcattgcgttgtactaatgctgccatgatgatcgaaatgtgtacaaccatctgcatgaagcccgaaaaccgttccgatatgctgatccttgacgagtgccataaaatgccctctacgctcctagagcacacgatcatggaatacaatacaaaggccgtggaggggctgcgttctatccctgagggcaaggagatcgtctctaccattggtgatatcgtagaacgcaccaaggactatgtcctgggcaagttatattctttgtctggtgaactacattcaatgtttgaagacctccatctcaaagtagaaaatttgttggaagtccttgaagaattggtcgaagatgatcgcttgagtgaaagccaagtgatgaaactcgcagacattatagatgtcttgcataacttgagtgattattgcggtattatgtcacaaacccaagcaagtacatttattgtgcaagaaaagggggaagattttatccggtttaaacctgttatgccttctgatgtaagtgaattcgggttattccgaaaagccgattatcacgttcatatgtcagcaacgatctgtggtatagattcatatgctcgttcattgggtattcgacagggggattatcattccattcagatcggcaaccctatcccgattgaaaacagaaaagtcaactatatgcccatagtgaagatgactaacaatatgggtgactatgaaatgaaaaggttgacggaatatattgatgaaataatcgcgttccacccaggtcagtccggcattatccatacagtgagttatgatcgcgccttggcaatacaaaaattcagcaaatatcagaattttatccatgttccgcgtacccgaaaggcgttgatggatattatggaaaatgcttttagaacaaagtccccatgcgtcattgccagtccggctatggaagaaggttatgatttcaaaggggattacagccgtttccaaatactgatcaaagtcccttatgattatctgggcgatccactgatagcacatatcaattcagtggatccgtcggcatatttccgcaatgcagtactgcgcattgttcaaatgtgcgggcgttcggtcaggggtgttgatgactgggctgcgacatacatcattgattcatcctttgaatctcttatgatgcgcaacccagaattcttcccaacctggttcaccgatgcagtgtttgaggtttaataacccgcaataaacgataaatggcacttgatatttggttcaagtgtcgttatcgtgataatcatattgtccacatcttggctgaaagatatatttgcgatcggtttggtttgaacatgattatacaggctataaatcagatcttcaccacgataaccgattttgccatcagtgtcctgccaatgaccgagggtagactctcgagcgtgtactcggaattcgaccatatgaaacaatgcttttgggatggtcttcagtatgctggtcccaacaaccggaatatcaatagaagagtcgttggagtttccttcataaacagattcccaaccaaatattggatcacctcctgccacaggagttcttttccagtattggaattctgggttgcccgaagcgttcttttgtaggaggattacttcaccgccttcaaaattggaagtgtctatatcgtcccttgctaataaatctgccggagaaccaataaatttccaagtttcgcgatctaaaatagacttataataatcggtgaatacgttgctgtcgttagctgcattgattgcgtctttaagaccttcacctgtgaccatctcattaagttttagccacatgtttttgacagctgacgcagctgcgacctgaccttcagtcccacccgccgtgtagtcatatacgatgtccagcttttggatggagtcgtttatggacaagagcaaacgctcaatttctggggcggtcagactgaaacgatatgcgcgttctgacatgattcgttcctcatcgataaaaccagtttgggatacttatacatgattgaaatatacactgacggggcatcgtcccctcagaagacacgagccgctggttgggcatttgccatcagtccaatcactggagagcaatggaaagtgttttatgggtatttacccccgccgtcaacgaacaatatagctgagttactcggggttataaacgcgatgaagatcctatggaaattctctaattgcggtgaacgctgcattcccccagcccgcattatatccgacagccaatatgttatcaatagcgttttggagtggcgtcgtaaatgggaatatgaaggaatgccgcctaagaacacagaacttctgctcgagttgttcgattattatgacaaagtttgttcaatttgtgaacttgaattaaaatgggtgaaaggtcatgcaggaacgatgggcaacgaaattgcggatcagtggagcgttagagccaaagaagatagttcaatggttcttgaaaataaccgtttctcggtcaagaaagtcgttggttctttcaatgaattcattggtatttgaggattatcacgatgggtatgaattttgtagacaggggtgataacgtcaccaaatatttcacagacgaagataatgaccgtgttgttggaatattgagagattggattccggctcgcaaaaaggctttggccgaaggaacacccctaccaagaatccccaattatgtggccatgaatgttcagatgattattaagaacatgagtatgcgctacaactatcgtgattatccataccgcgaagacatggtcagtgaggccgttgtcaacatccttcgttacctccatacatttgacgtcagtcatatcggcaagaaaggaaaaatcaatttcttctcttgggtaacgatgtgtgccgaccgttcattcgccaagaaactcaccagtgaagaagaacacaattatatcaagttgcgttcatttgaagaagcaggtggtttcgctgcattatcagatgacccagacttccagcaacagacattcgtagacagcactggtataacaatggacttccgtgagcgtatcggaaattttgaaactaagaaagaagcccagcgcgaaaaggaacgccaaaagcaaatcgccatcaaagaagaggaaaagaataagaaaattcctcgcggtatacttcaatgtctcacaaagagtgaaaataccattaccgccgacgccgaagataattcagactatgacttcggctctactcaattcaatctggaagacgaaatatgttctatggaaccagattgggaagaagcaaagaaacgagcattagaacgggagcaacaaaataatggcgattgctaaaataggcgatttacatatcgggagccgtaatggttcccgttacgttcgagaattcataaagaattatctcatcaattattttattccagaattggttgacgccgatattaaagaagtgtggcaatttggtgatacgtttgatgttcgcaaattcatgtatggtcgcgataaagattggttgaaagatgaattgacgccagcgctgcgcaaagcaggtattaaatggaatggcatcgtcggcaaccatgatatcacgttagaagagtccaatcgcattaactggccttcttatcttaatgaattggcaccggatgtttatcattattacagcgaaccaactgaagtaatgattgaaggtgtcaagaccctcctactgccgtggattaacaaagagaactatgatgcgtccattaaagcgatacaagatacagacgccaagtactgttttgcgcatctggagttggcagggttcaaaatgtaccagtcctctgtatgtgataaaggtcagatcgacttggcgctcctttcgaaattcgagcgcgtggataccggacatttccatacccgttcgatggaaggtaacattcagtatattgggactccttatcaccttacctgggaagaccacaaagacgggacgaaccgaggattctatgtagacgacatgaatggcggggaattgtttatccccaacaacgaacaacaaacgttgttccggttcgtggaatacgactacacacaaatgtcttctgatactgaaggtaattggatcgaccccgattggttaaacaatgttttgggtattgaagggcaaatcgttaaggtgattgtacagaaccgtgacaatgccaagcattatgaaaagttctgtgacgcattgaagcgctgtaaatgcatcgattacaactttatcgatcggacgattactgttgcttccgagaaggtagaagtcactgaagagatggtggctactgatgctgtagaagtcttgaaaaatgatatccgcgcagccaacaatatccagcgccctgaaaatgtttgcaaacttgcagaacaattttataatgctgcccaacaacgtttgaacacattggatgcataataatgaacaatgtagacctattatcaaaatttggttcgctgttgaaagaagcggacactcctgttactgttgaagtaccatttgaaattcgtggggaaattacccacaaactgactttccatcgcggtcgcgctaaaaacttccgttccatcggtaatgaattcatggaaattgattaccaacgcaaccctgcaaccctagtcacatctgatgacaatggggcgggcaaatctacaatgcttgtttggttgttattcttcgttctgtataatgacacatacagcaagaaagaaaagaaagccggattagtcaattcccaaaacaaaaaggaatgcgtgggtgaagttgagttctccactcgaggaagtgaatggaaagtccgtcgtggtatcaaacctgatttcgtagaagtctatcagatggtagacggtgagtggaagcaggtcgtgaatgatgcggccaaagccgacatgaacaagtacatcgtgaatctcatcggcgtggaccagaagatgtttgagaactccctggttctgggcaaagagaagttcatcccattcacggaaatgtacactgccgaccgtcgcgctatggtagagacaatctgggatttggggttcttcagtctgatgaatgaagatgtaaaagcatctatcaagacagtaacgaatgaactcacgacggtcaccaatgattacgcctttcatgatgtgaatctaaaaggccagcaagcgcagttggaacagatcaataattccaatgcgatgatccagcaacaatctgcggatatccttgtccaagagaaggaacgtctgaatagtttgaattcagatattggtgcttctcaagaagaactgacggaattccgcgcccaagattctaaattagaaactgaattatcagaagtccgtaatcgcctgaatgccgaaatgaaaggggatatcgatgccatcaatgaagagtatgcggccaaaatccaagcagttcaggatgaagccgataccaaggctgaagattatgaacgcatagaagtatccgacggcgaaaggtcgttacaagaaatccgcgaaagaatggcggtggtcgcagaacgcaaaaacgaactagtcggtcagaggaatgctaatctcgacgaactgaataaagcgttggcgcggcgtcaacagggagagaacttccgtttcaaattcgtcacagaaatggaaggacatgaatctgccatcaaacgtttccacgatatgggcacttgccctacgtgtacgcaattagtgtcggacgataccaaatcacgcattgaaagtcaatattatccacaaatcagtgaactgactgacaagattcttcaggttgatacagccctagaagaagtgaattccctgattgagaattacaatgtcagagattcagaattatcttcccaagtttctgttgttgacaaagaattggaatccatacgcaacgaagcccgtgaagcagaaaccgccatcgcggctttaaagcgcgatatccaggggttctacgaagttgcggtggttgagaaagcatctttacagcgagaacaacaatctaaagttaatgatatcctcagagcagtagatgtccgctatgaagatatcacgacgtcaatccgtcaagcgcgtgagcagctctctaagagtatcaacgacacggctgataaactcaccagcctgaaatcacgccgtgcccctctggaggcatccattgccgatttagagcgtaaattggcggttaagccgacaccaactgacgccctggaaggagagatcgctcgcatcacggcattaatggaagatctgaatgcgcggcgcgtagagttggatgagaaattgcaagacctgaaccatcttctgttcttcctaaaagatgatcagaccaaggctcgaattatcagcctgtatttgcctttcctcaatagtaaaataaacgagtatcttgaagcactcaatatgttcttggatattgctgttgatgatacatttgaaatcaccatgagcgctgcaggtcgtaaagggcaaagcatcttctcgttatctactggtcagcgtagtcgcctgaacctggctgtgacattagctttacgagatgttgcaaacctcaaggcgtcggtgcaatgtaacctgtttgtgctagatgaaatccttgagaacatgagcgagcgcggcgtacaagaatcggttgagatgctgaagcataaatttggcgggaacaatctgttcgttatcagtcaacgcgagcaggaattccaagagtatttccaacacaacatccgttatggtctgcgtaatggcatgactgaagtgattaagaaggattgaacatgaactgggatcatctgtggttggctatcagtgtctttatgttctttgatgcgttgttgggaacgtacgagatgagtagggagtccagagtcggggtgatggtttgttgttcccttatcggcgtggtatccctttatttcgctattgggtttgccgcagagatcttcaaatgaaagttgtccacttcaagaaggaaccatacgacgtctacattggccgtcctggcaaatggggaaacccgttcgaggtcaaagaccacgggcggggaaactgtattgagttgtttgaagacgacttgtatgtgcgcctgattgaaggggatatcaccgaagatgaactccttgaattagatggaaagaccttgggatgctggtgtaagccgcgcccatgtcatggagatgtttatgtcaaagtgatcggtcgtattaaactattccgtaagttgggtaaatcttttactgaatatttgagacaaacatatgtcagaagagaaagatgaaattcagattgcttgtgatatcgctgtcgagttatggaatcagcgaaacaaacattatcaggtgacgttgaccccaaagcgttatctagagcaaggtggcttaatagtcgttgatgccgtgttaggtttcaacaaatatactgacatgcaaatcttggacggggatttcactattgcttccaatacagcttgggatgatcctatggaatttgtctacggttggttggaatgcatagaggaatcataccatctgatctacgaataaggtgaagggcactaaatatcacgtgccctttatcttttaaccctgtcaggaggctccagcgagctgcaagtcgacaaaccacataaggaacgccatcatgacgatgacaacgttgaaggccatggccttgtccgtgacctttaccattgccagcgggaatgtgcacgcttcccaaaatacaagtcaatgtgtttatgatttctctgagcaacaacttaatacgctggcaactgcctatcacataggcaagacgcaagaccttggttatactctggccgccatatcttggcgagagagccgcgctggtgaagatgtagtgtcaatgcgatacggtctcaaggcggccaacatgggagcctttcagaacaaggtgaagacagttggtgatcgcgagggttgtaagaatcgcaagtgctatgccaatgtcgccatcaagttgttgaccgaccaagaatatgccgcgcaagccgccctcgatgagatgcagttctggctgacctatcacaagcagaatgtacgcaagtctctttcctcttacaatgctggcttcgcccgtaatcccgcatcgaacgcctatgccgccgacgtcgtgaagaaagctaaatatcttcagaaatgtgtgtccttcaaagggcgttccatctctgaggtggatcctcaggtgatcgccatgaacaagcgcacgatagagaaactgaagaggacaagataatgaacatcactccatcccagaccggatatgaacatatcttggtcttcattgccctacagggtgcgaaggaaggcatcatcgtatccgagtattcaggtacggttcgccttgctgactacgtcgcgccggaggcattcattaagaagtgcattgatgcatggtgcctctatgccctcggcacgtatcctgctcctcctgatggcgagctgttgtttaccgtaccgactccggctgctggcgatacagacgcgatctttgccgatatgctggccaaggggtatctggtgtatgacataactcacgatgtctattccttcacctccctgaacactgtgtttgggttcaccataacctctgaagccaacatccctgactggggtatcttggcctggggtgttgacggcctgggcgcaacctacgcaacgtctgatgttggtcctgttctgattgatgagggtgtaaccatggagcagtaccttgctcagttcccccaacacaacatcacgggtagcgatatcattttggtccagcctggcgctgttgccctcccccgcctcgtggacggatggaagcctcagctgaaggacatcattgcagggatcaggtcccatattcctgtggtgaacccaacaccgtaaaataattcaataaaaggggctttcgcccctttacttcttcaatatccttccctatacttcattccataagcagtacgacaccaacttgaaacgaaggaactatattatggctatcaccacaaaaactgaattgaacgcgatcctgaaagaagctggcttcggtagtatcaaacgccagggcaaggttttacgcgatggcggtaaaatcgtaaagggtatcatgttgggaagtactggcccgttgagcatcagctcccctcagtatgacaatcttcgtaaactggcgcgtgaacttctccaagaagacctccatatcacgacgcaagaaaccaattatgtggttttggaatgcaacaaattcatcgtcagcctttacatctgcagcacccgctcatatccggtgtctgaaggttatgacccaatgtatctgacccattatgctcaggttactgtcaaagcaaaataatcaaattcaacggggagcttcggctcctcttttgttttcgctaattaaggaaaaccgcaccgatttattatcaggagaattacaatggccatcccatctttcttgaactttcttgaagagtctaaacaattagacgaagcgtttaactcatccccgtatgaattgacattcggcaagaagaacgctggtgacattttcttcacatttgttgatgaagacgaaaaagaattccgtattcaattctataccccacaaggtctaggcaagaatgttcgccaagtctttatcggtcagaagcgtggttccacatatcccgatgccatcggtcgtttcaagaatccaatgcgtgttatcgcatccatgattgaagcaacaaagcaattcatggcgacgcctttggggaaaaccattgacggctttgcaattaacttctccaagaaagcattggagcgtggtgttaccctcctgccgaagattatccgtcagtccggcctgaagcaaaagctcaatgtcatggacctgacgtatacccctatccctgatcgtgcattcgtatgggttgtgcgtaaaggcaaggatcctgctcaggtattcgatggtccgaaaatgaaaggtgtcacatgggatgatccggacaaagtcggtgacgtcccggttcagaacaatactccggatgtgggagttgtggctggttcttctgaaatcgaagtcaaccgcgatgacaacggttggaaactcaccaaagctagtcgtactcagcctatgctggtctggtctggtaaagaacgtgggcggactattaccgccaatatcgcacctcacgcggatatacctggggtgtatgctggtagtatcatgaacgagaaacgggtccgcgctcaatccccagacgctattgtacgccaactgggattaccgcagatccctgtgaatattctgaatgatttcaccaagcagtcgtccatcttctggaaagagatgacgggggattctggaaaaatagacatgcagactttgccaaaaacaggatctgtggatgacttacgaaatcagggttatattgctcgccttctggggattgtagacacatatgctccacaacaggtcggtatgtatgcaggtgagaaactaacacgaatcggcgcgggcaaatatcgtacttcgggtggtctggacattcaggtttatttctggggaattgtcaacggaaaactttctgtagaagtagtatcagatttgtctggtcatgaccaacttatgctggaaggagacgtgaaaaacgccgcttacgaaatagtgggcgcaatctcccgtgcagccgattctcaatttgctggagaagtagaatgccgtgtggttcctatcaatggtgccccatatccgattcaaaaaatttcccgttctcagggatattcttacgttgattgtggttctttcacattgaagatcgccgacgctctgctgaatcggtttaccttgggcaaggcggtcaaacttgatctgatgaaagagtttcctcgtacaacattcaaagtccgctatgattggcgtggaaaagatactctgttggttcaatcttccgacgagcgtactatctttggtgaaatcaacaccaattccagtgaaaatgcagtgatcactgcaaacatcaaagtaccaagcaacgctaaattttttggtgaaaccgttcatggcttctctgtagcgtgggatttccagttccctgctggtagcaagttgcaagccgcaagattcgttaccaacgtaacttacgattttgatcgaaacaaagccaacatcaccaccatgttgactgaaaatggtcgaaatgtagtagacagcaatggatatgatatttcgttgtctgtcaaatcagtgcaagatgcattggatgagatttctcgacgcctgaaagacgcggcttcgcaactgagccatctcagttcatcaaactataaccctaacaatctgttcctgaatgacatcactgtcagtaaatttggcgcgattatgttccaagacaaagatttggcacagtccaggaaccgcacggcagtcattgatgctttggctcgggcgaaagatcaggtcgctggtggtaagaatgcaggtgatctggccacctatgccgaatccattcgtgacagcgctcctaatggacgagatctggattggcaggtatacacagccagtaatggtacagttctgaatgttgcttgggatatcactttccgtcgcaatacgaccgaaggagcatatcgtgaattcaaggatcagatcaaccgtgccaaccaatacctgcaaacggtatacaacgacgcgaaatctaaaggctataatcctaccgagccaaatctcatgactcttgaacgcgctcgccaatccgacgaatgggctatgagtaatggtgatagtgcatacagtgaatatgaacaatcgctgggtggtaatcttcagattagtctgaaataagtcatagccccttcgggggctaaatacatcaaaatccttaggagatccacgatgaaaccgtttatagaatattttaaagaacaagctcaacaaccgcaaactcatggcggcatacaaggcaaaagtgtaaccatcaccaaacaggccgacggaacccaatggtgtactggtgccaccgtgacccagatcactccagataatcaggccgacatctataatctggaattgactaacggcaacaccaccaaagtaaaactcacaccggaccaggttcatgcagtttctgtcggtaaagatgtggttgccgttcatgacggttttgagttcttctttgggaaagatgccagcaaaggtcaggtcacagaatcctatgcccaagaattggcgaacatggataagatgatcggtaaagcgctgggcgttggcaaatctcgtaaaagcggtcgtacagagtttgaatattccggtattcctggtattggaaaacctgtcgcaatcgacttcggcggagaagacgatccatatgtaattgtcacagtcgctggcaaacagcatgacatcagtaaggggaaaaccctagctgcggacattgccaaatatattggcgttaaacctgtctaaggaggttgtatgaaaggattccaggacttcctcttgaaggaagcaaaggttgaccatcgccgtcttggacagcagggcatgtatgacatcaccgacgttggccgccctaacaaaggcgatctgattgattactatgatcgcaacggggataaacatcaggggaaagtgaagtctgtgaacgcacagaacattatgaccctgatgaacaccagcacgggtgaaaccgtcaagctgactttgatcaagccttgatgacaatccccctaaatacagggggatttctttttctgaggaaacaatcatggttaacgcgagtatattcatcaaccgtggactgcttgctatagcccactctcatgccctccacttcgttacaacgtcctatgccaagcacaaggcactcggggagttctacggggaactggaggacttgctggacacctttacggaagcgtatattggtgcgggaggtatatacattccaggattcgagaacatcaaattgtacaatcctgaaccgatatcgtatatcaacagcgtggtcatcgacgtcaatgggatctacaatcagtgtgatagccatttacaaaacacgcttgacgaaatcaaaactttgtgctatcaaacgatttacaaactgaagcaattgtcctaactcaataggatcatcatcaggttataattttgcccattcgtcatcaataggatacagataacaatgggcaaaatctttataccttctcactcagaaggttgtcaaatcgctgcaaaatttctacacaaacaaatgaattgtggtgctgtgttcgtagaaccaaattcattcgataatcgtgaatcccctgacgccatcgggttcagacctgggggttgttctatattaatggaagttaaagtctcccgcgccgacttcctgacagacaaaaagaaacctcatcgcatgaatccttccataggcatgggagcatatcgtttctatgtttgcccagcagatgttattaaaattgaagacctccctcccaagtggggtttgttgtacttcacccctagaaaatctttgaagcccgttcacgttccgaatatgcaatattcctctttgtcttcaccggaacattatgcatcatatttgtctaaatctttgggcaaaagaagacctacagatatccctccttatctattggggtacaaacagatgctggaagaattcgcccactttgaacgcaatcaggttgcggaacaaaatattctgtatggggcttggcgacagctttgcatagctcaaagtcgcggggtggattataatgtgacagaagtgttccagaggccaaatatctaatgaaattcttggacgaacaatacatcaattttctcgccccccgacttgataaattcagctgggaacgtgttggcgctgtcgccaaatttcgttgcccattatgtggggattctaagaagagtgccaacaagcgtcgtggtcacttcttttatgatcgtgatgacgatgtgtttcgtttcaagtgccataactgtaatgaaatgagcggttgggcgtttgaattctggttgaagaagtttgacgagcgtctgtataatgaatacaaccttgaaaaattcagaatcatgggggataccagttcccgcccgttacaaaaactcaaaccactacagcgcctgacccagacagcgcgtatcggttctcaggtcgccaaacgggatgaagagcaccttgggaatatgatacgccttgatcttcttgaccgtgatcatatcgcccgtcgttatgttgaaggcagggggatgccggagagcacgttatctttgttgtattacagcagaaattttcgtcaagatcttctgagtttcgaaacggatgttgagaagcaaaagaagatacccgaagatgagcggctggtaatcccattctggacccaagacgggcgcatgaagatagttcaggggcgcgcattcggcgataaccttcctgatggggtattacggtatgccactgtcaagcctaagaacgaagacactaaaatctacggggaagagcgcatcatttggaacaaaacaaagttggtagtagaaggtccaattgatagcttgtttctacctaattgtcttgcgactgccgatgccgacttattaagtgctaagggagacatctatatccccgataaccaatacaggaacaaacaagtttgtgacggtatacagaagatgatagacagcggagtcaaagtcgttctgtttccaccggaaataccgtggaaggatatcaatgatatggttcgccctgacaagggaaatatcccgatacgtgatctgcttcagatcatagccaagaacgtatatcagggactgtcggcgtccttacgattttctgacttgaggaagatatgatggatttgaaagaactgaatagtatggacgatgaagagattcttcgcgggtatatgcaagcccgtgaaggatatgtcctatctggcttagaatctaagagtttcatccatggttggaggaacggaatggtggacttcggcggttgtcctatcactgaagaacaaatggatttggctcgccagtatttgaaaaagaagccgacctaaaactttttaactcaaattatttcccttcaggcctcgttctatgaggccttttaaaatacttcaaaaataatttaaaaaattattgaaaaagactttactcttcaataatgatgcactattatagttcacatagggcggtacacaacacactgccgaatgaaaagtgaaacgacccaactacattatgaggaattacatcatggctactaccaaaactctgatcaccaacggcaccatctcttttgagctgaacaccgaagttgcaaaagttgagatgttccgcatcgctcaggcagctggcttcactggtggcaaaacttccttcatgaatctgctgaacggcaaagtgaaagcgaccaacggcttcaccctggtagagcaggttgtcgtagacaaagccgtagttgccaagactgctgacaaagttggcatgctgaaagacctgggccatgacatccacgttgtcgaagcgaataccgaaacttacggcaccatcgtagtcggtaaaggtcgtattcagctgaatccgctgaacaacggttccttctctgtgatggtcttccctaagaaaggctacgcaaacgaagacatcgtgaaagccgctggcggtgaagcgaaatcccagtatgtcaaaatgggtaaactgaacgccgacgcagtagaaactctggtgaacaaattggcttaaccgcaagggggaaacctccccctttcaaagttttggtaggagaatgagacatgagtagagcaattatccaagcctctcctggcgctctatgggttgcagaccgaattcccaacgtgcactttcctcagttccaagaggaactggaacaagcgatgttgaaaatatttgaaaaatatgggtatgattccgaagttaggacttccttcaaagaaatcatgcctgtcgtcgtagtcagttaatcgacactaaatcctcaagttcgactttcactttaaataccttcgcatacattgcggaggtattttcttatggccattctaaaacttggcaaccgaggttctgaagttaaagcgcttcaacaaagcctcaacaaaatcggtttctcccttacagccgatggcatatttggtaaggcaacagagaatgctgtcaaatccgttcaggcaggtgctggattggttattgatggtattgctgggccaaagaccttctatgctatccgcaacgctggagacgctcaccaggaacatctgaccgaagcggacttggttgacgcagcacgtgaacttggtgttgagctggccagtatgaaagctgtgaatcaggtagaatcccgtggcacgggttttaccaaaactggcaagatcaaaactctgtttgagcgccacatcatgtacaaaaaggtgacggccaaattcgggcaagcaagagccaatgctctgtaccaactctacccaacattggttaaccccaattctggtgggtatatcggcggagacgcggagttggaacgccttcagggtgcaatcgcccttgacgaggactgcgcttacgagagtgcttcctacggcctattccagatcatggggttcaactgccaaatctgtggctattcaaatgccaaagagatgttcactgatttcctgactggtgaacgcgctcaccttctggcatttgtcaagttcatcaaggcagatgccaatatgtggaaagccctgaagaacaagaattgggccgagtttgctcgtcggtacaatggtccggcatatgcgaaaaaccagtatgatactaaactggcggcagcatacaagagtttctgctaattctcaaggccggaaacggcctttttctttatctgatatacaggtatgctgggcgtgtcgcttaattaaaggtgtcgacaacccctgtagatataggaaggttcaacatgcaatctgcgtctaaagttgtatccatgaagccagcgaagacaaaatctgctcgtaagaaagacaccatccagaaagaagaagactggatgaaattttctaaaggtgatttcaaaattgctccgttcaatggcctctcagaaaatcagaacctcgcatatcaatccgcactcaatgaacatctcactatcgctatcggtccggcaggtacaggcaaatcctattgtggtgcatctgctgcagcaaaacatctgattgacaaaaccatcaacaagattatcatcacgcgttctccccttccaactggtaccacagcagggttccggcctggtgatacatatgaaaagttgatgccttatttgatgccgctcatacagacattcaaaaaggttctcaaaacagacacgggttccgacggtttcttcaactatctgtgggagaaacgcatcatagaaattcaagaccttgaaactgtaaaggggatgactttcgatgagtgtttcctgattatcgaagaagcgcaggaatgcgatatggagcagctgaaaaacttgttgactcgcgcttcagattcttcgtacatcttcgtgaatggagatatcaaacagtctaacaagcgactgcgtgatagtgcgttacagacgtatgtggattcctttaaagacttcaacaacaagctggaaacaggttccttgcaaattgacggtgtagaaatcggagatgaatatccagagtgggttcaaccattcagcatcatcgaattcgataagtctgatcgtaatggtcgcggcaacttcacccgcctgatgttagaaattaacgacctgtacaacatttaaataccacacgaacttccccgccgccttcaatccatggaggcggcgtataataacagcctgcataaacacaaaccgaggatcgctatgattaacatcataaagcgggacggatcttctgtcccctttgacattgaaaaactccataccgttattgaacgagcatgtgacggccttgaaggagtatcggtgtctgaggtggaagctgcatcgaagattcaattcaccgataacatgaaaaccgaacgcattcaggatatcattatccaagctgcggccaccttaatttctgttgataaacccaattatcaatatgttgctgcccgcctgaagtcgtatgacctgcgaaaggttgtctatggtcaatacaagccacctcatttgctggatatctttgcaaagaacaccaagctgggtgtttatgaccgcgaattcctcgaactttactctaaagaagaattcgaagaacttaacacagtcatcaaccacaaacgcgacaagaatttcacttgggcagcaatgggccagctgactcaaaaatatcttttgcgtgatcgttcttctgacagcaaagtgtattatgaaacccctcaagtcatgtacatggcaatcgctatggcattattctcagcgtgggataaagaaagcagactgacaatggtgaagaagttctatgaatacgccagtacaggcaaattcagtcttccaacacctattatgtccggcgtccgcaccccgacccgacagttcagctcttgtgtattgatcaaaactggtgatacattggactcaattaatgccactgcgaaaagcatcgtggattatgtttccaaacgtgctggcattggattcgatgttggggcaattcgtggcatcggtagccctatccgtaaaggggaaatggtccatacaggtctggttccattcatcaagtatttgactggtgccctgaagtcttgctctcaaggcggtatccgtggtggatcggcaacatgctatgttcccatctggcattatcaatttgatgatgtcgttgttctcaaaaacaaccgtggattggaagagaaccgtgaacgccgtatcgactatgggattcagattaaccgcgtcatgttcgagcgcctggtgaacaaacagcctctatacctgtttgatccaaaagacaaccgcgaaatgtatgaagcatttttcgcagacgtcaataaattccgcgaaatgtacgacaatatgatcaaggccgccgacgccggattatgtcgttccaaaaagctgcaagctgaagaagtgttccaaatgctactagaccagcgttctgacactggacggatctacatcgcattcgttgatcacatgaacgagtatagtccgttcaatttggacacgatttatagctctaatctgtgtttagaaatcgcgcttccgactcgtgagttccaacagtatgatgatgaagatggtcgcattgccctgtgtacgctcgcatcatttaacctgacggcatttgaagatccaacagaaatggaagatgttgctttcgttctggtttcagctttggatatgttgttggaatatcaggactatccagctcgccaagcccgtttggccgtagaagaatatcgtcctctgggtatcggtatcgtcaacgtcgcacatttcctggccaagaatttcacgggttatggatcgcctatcggattagaacttctagacaagtggatggcgcatcttcatttctacttggtcaaagcgtccaaccgtctggccatgcgtttcggatcttgtaagaagtcaactatccatgattccggatttgtaacagccgaccttcagcctctcccgctggacatcctacccaatggcaaaaagcctgttgggcaagcctatggtcttgactgggaaggtctgaagcagaacctatccgagtatgggatccgtaacgccacgttgttagccgtagcaccaactgaaagctcctctcaggtgctgaacgcaacgaacggtatagagccaccaaaaggtctcatcagcatcaaaggcagcaaggacggcgtctataaacagatcgttccggatgtagaaacccttggtcctctatatgacctgaaatggaatctggattgtattgaatacctgaagacggctgcggtcatacaacgttgggtagatcaatctatcagcaccaacacatggtatgacccagagaaatatccagaaggcaagatcccgcgctctcttatgatgcaggatattctgtctttctacatgtggggtggtaaaactctgtattacaacaccaacaaagactccaaggaagacgaggagctgaaacaagttgaagaaatttgtgacacctgtgttgtttgattaggtctataccacacaatcgtttaatatcaggggtgattcgtcaccccttattcatcaggaaagaaacatgaacgaacaaaagcaattttcagtatttgacccatcttcagataatacaggtttaccgttctttggcgaccctgtaagtatccagcgttatgacaaagtggcttggccttttgttcaaaaatggtatgaaaaaggcctcagccaattctggcgtccggaagaagtggatgtcaccaaagacaaagcggactttgccactctatccgctgcggaacaacacatctacttcagcaacctgaaacgccaaaccatgctggactctattcagggcgctgctccgttcgaagcgtttggcccatgggcatcaacaccagaagcgcaatttgctattctggagtggacgcgccaagaagccatccactcactatcctacacgcatatccttcgtaacacagtgaatgatcctggtatcgtttttgaccatgttctggacgttcaggagatcgtcgattgcgccggacagatcagcgtatactacgatgacatggtgcgttacagcgggatgcgtatggctggacgcgcgttcacccgtgaagacatcatgaacgccaagcgtgcattctggcgtgctctgtttgctgctaactccctcgaaggcgttcgtttctatgtttcattcgcgtgttcttgggctttcatgcaattcctcaacaaaatggaaggcaacgctaaaatcatccgccagattgctcgtgatgagcaagatcacctcatcctgacccagactttcctgaaccgtctgccactgatggaccctgattttgccattattagagaagaactgcgcggcgaaatgacccagatgtatgtcgacgttgtcaaccaagaaaaagaatgggcgaattatctgttcaaagacggctcaatgctgggactgaatgcaaatatccttcatcaaatggtggactggttggcaacacaccgtatgggagctattggccacccatatccaggacaagcccgtaaagacaatccggtgccatggataaatgagtggctagataataaaacaatgcaatatgcattacaggaagcggaagcccctgattatctgactggtgtccttactggatcagtctccgacggtctgaaatttgcctaaaggtgagaaatgattacgatttattccaaacaaggttgtgcgcaatgtttacaagctgaaaacatctgtcgcattcgcggtattgaacaccaaattctgaagctagacaaagattacaagctggaagaattacagaaaatcactggtaaacagcgcatgtctatgcccgtgattgtcctggctgatcaaaccgtgaccgatgttactggactcgccgcaagtttgaaacgctgatctttcaaagcccctcaatgaggggctttacttttgaaaccttcccacctatacttcccagtaattcattggccaacgccacaaattgaatagcaggaactatatcatgaccaactctgaaatatatcatgcttctatgcattatcacgacctacgcgcccgtccaggacacgccaaacaaaaaggattctgggtggcagaactcagattacgccgtgctgtcgttaaccaccaaataatcatgaatcggaaaaacgacaaatcaccgttgttggcagattcttatcttgaactccgcacagcctatgaacaagccaaacagcacgtaataggagcataacatgtccaacaaattactcaccaacccccgccacaactttggttatatccaattaccagaagtgatagacacgttgtttgctgaagacaaggaagaagtttgttgggcaccagcgacccttattgaacgcgaaattttcaaatctgaagtagacgtgatgttccgtatcggttcatccggagacctccattatttggggaaagtgaccgagttaaagacctgccatgctcaaagatttttgcgcattgacaccactcaatgtgaagacttttcattcagcattctgcgtgaatatgttcaggtcaaccgtcctgtgtatcaacaccgtccttatttgacgtcgatgcatatagacaattatatttcagatatccacttgcgcgcctcaaccatacatgaagaggattctggtgtcgcggttgatacggtactcaacatgattgtgattacgttggtgccatgatatgtataaattcccgatattagagacggtgtattccgtcaataacaaagaagaacctttatttgaaaatttagataccctgttaaaggaaagcgcacatgttcctcttgtcctcaaagggaaagaaatagggtacagccatgactgtcaaataatgcgttctgaaggcagtgatatccttcgcattcgtttaacatttgtgttgacggggagactagatggtgtggtggttaacctaaagccaaccaccatagtccatagaataggtgggaaattactgtaccgtttactgacgcattttgaaataattaccaaccgctccaataccaacgcaccttaatcagtttgtgcgttttaaccatatggtgtctgttatcggctctgggttgttgaccacatagaacgtgatggttacgcccagagaatgataatcgtcagacaatgatacatcaacagattgcaattcagctcttggctcaaaaagagcaatggcatcttcaactttgttcttcacgtcgacctgtatcgtgggatttgtgttttctcccagcatggtatacaaccccgccccaatactcggatatgtcggccaatcacctaccgtcgacatcactatattacgcacagattgtagtacagcataaatgcctgttttcttagtgacatctttggtgaccggatgcatgccaaacttcaggtcaatgtccttgtactctttcatgttaaacccctttggatattgcctctccagcacgagagggcattatctgccctcaccagtgtaatcatgcgcctttaccaccaaaattgccgccgttatcctctaactccggttttatatcaattggacctgggcctgtagttgttacaggagttgcccccgcacgtgaggcggcgcgaccagcgccttcagcatatttgatgttgccttcaatagtctgtgcaaccgataatttatcacaaattatttcagttgccagtatcttaggaaccttcaaagtgttagacacttcaaacatttcacagatgagacgcaacacgttctgtgcctgtatctcagccaattgggaaaatttcatcaacgctgttccagcgaccacattggaatagttagaagagtgtaagtgataaacttctcctgtcttgcgctgtacttcagtcccaccaattgtcaggttatgatcacgccccacataataacgtttgtcgaacatcgtcaggtcataatgatctttgactgatttattaacaacatcaccgtcaggaagcatttgcttgtaagacccagaagagtgcatccaatgcaaacgttccccgcctggagtgtcatcaacctccataatatgcccagcgcgagaagccataacattgttgtatggataacgagaccccccagccgaaggaaccgatacctctcctgtgttgtcaatatcctgaacacgaccagaaggaggcggtgtttcttggactttcggggtacgatctggaataggttcagaccatttaccgttttgttgtgccggagctgctgaagctgtagttgctggacctggccaacgataacccaacacggatgagcggctaaaacggctcaccttcacagaatcggattggtttcccccgatacaccaaacgtagttggcgtcaaatttctgaacgaatgcaacgtgaccgaatgtcgggttgttcccgcgccggaacactacaacagcgccgtaacgtggttctgacagaggagacccccattgtagatatgaacgagccaaagcagaacgtgttgatgtatatccggcctgtatcaggacccatccaacaaacgacgcacaccaactaacttcatcttctgacgcccccagggaagttgtcttatgatattccaatatccttgggttgttattgaacttaccagaatattctttgacgcccaattccccacgagccacggtcatccacttctcaggatcatatccgtctactggtggttgtggatccggttgtggttcatcctcaatcttaacaggaacattctcgacggcattatacttctgtctttctatagattgaacgacctgacccagcgccaatgggtttgtatctgacccatctgttggtgttgctgctggccatacccatgcaatacggatgttctgatatgcttcatccaaagcaaaccccatgacgtcagagccgacagtgatacccgtcggagaccagcctaaacctgccgaggatgcgttagatgctggcataagcattttaccccaaggcaataattctgtaggcaggagagtggtatcctccgtgtgtaccccatagatgcgtactgcgacgcgcccgttttgatcgggatcattcacgtcttcaacgcgcccgtaaaaccaacgcaagttgtctaacatattgaaacccattagtcaattagaatcatgtgatatttatggcggggatttgaaatctgtcgaataatatattataggaagctctgcttccgttcaacctttattcttttcgttcctactcgctgaagctcgtaacaccgccttgacaggcagtcccattccaatgccatggaggcataaaggcgaggaaaatactgtacacctcagaacatgcgtagaaaaggattatttttgcttcaaaacaaattcaaaccccaattacacaacacttacttcagcaacaacggtgttatagcaatttaaggttaaacgttcctgtcttaacctataaaacaggtaagcaaagaaaaccccacattacgtggggttggaagatttcttagacaatctaattttcagtgtctcggtaagcaaatcaagatgccacggcatcgattcactgatttcaaatatgctatagccgtgtgcctttaattcgtcacacatcacaaaataggagagtaaatcaatttcaaaaattaaatgaaaatctcggtgacactgttaaatttgacttcgtgttctttcccacacgaagggcatttaatttttgtcgcgtaacgaatacgcggaatctttttaaagaaatcattcgaaatgtcctgaacaatctcagattcaatattgtccccgacccatttgacaaattcatccttaatgcgttggcgttctttagcaacttctggatcgataccaggttcagccggattttccaccttccaaacctgaccatcgtcgtcatacagacaatcgataaaggtggcaatcatttgttcaacagaggatgcttcgttgagcactgaggcatccgagaaggacggctggcgcatctttatatgataaccacctggtagatcaaacgtctccctgaagccttctggggacacgcattttacttgattgagcgggatcggtaacacaagctcttgaccacaatctttaaggccagaaactggttcttcaccaccttcattggtttcggcttgaactttgttattacatttgtaacgtatcttcatgacctcgccgatagatatacagcgcatctttaaaaatacttcttcagtaacaccgattggcagtttactaaagggaacgccagcatcaacacaactgtcaaatagttgctccaatgttgccacgcgctcactcataggagtgttcggatcagcaacctgaagtaacatggtctgttgccctgctgtaaaggcgcggtatttgatcacagtcggccagaaatcacttttatgtgttctctcagttttgggcaatgatggtaaattcataatgtatctccacggtatattgtaattgtatttataacgaggactgaaaatgcacgcgttctgtttcagtaaagtgggggagcaactcgcaattaattatccggatgcaaacacggacttttttgtattactcacagattttgcaaataaattggggataaagaatgtcgccatgaccgtgtccgatatgaaattcgggtggctgggagttaaagatttattgaattatggattcaatgtgattatcgttgatccaacgtacagcccaattgaagaggatgatgttattccggtttgggtgcaacgagatattaacaccctggacaaacactatccggattgtatcatcatcggtgaattaataccgtattttcataaacgtcgtcgccttgaaacaattaaaagttattttcaagataatgattgtggatataaaatggatgtccttacgagcggttgggttgtgaagaatactacaggcaatgccgaccgaattacttcccgtctttatgaatatgttaaagaacgggattatgaactggagaacaataatgccaaacgtagaacgcgctgatattcagcaggtacaaaaagttatccttcaacgcctccgcgctgttatcacaaatgaaaagggagaaattgcgccagggtttgaaggcgcattcgacggtttcgttgccgatgatcaggggaaccccgttgtacaaacgatcgctggtatgattatgctgaatagtgaattcatgtcggaagggaaaattcattattcgccgaaccttagtgttgatggtgagtctttggcttccgaagtggttgatttaacggttgccgttggtgagttgggttatgttttgatggtttgcaccgcgcattatgtggacgccaacggccatgtgtcatatggtgatgaagcgcgggggatcaaacgtcatgtggacacttctgcattactgcaacagatacgtcagatgcaagaagaaatggactccaaacctcgcctgattttaccggaaagcaaaatcgtcaccagataatccggtctaaatacccataacacaatgaaacttcgttatgggtatttttatgaaattttgcggaatcgactattcttacggctgtccagccatgtgcttctgggacgacaaagatcctctggattttgatcatcttcatttctatgcacatcataccgttgagaagcactgtcgtcaagtgcgacacaatattcttattttacgtcaacccaaatatgaatcccccgaagaacggttttataacatttccaaatgggctgaagccgtacttcttacagagaagccggatttcatcaccctagaaggctatgctatggggaattcaaaaaactccaacaacatatgccaaaccgccgagaacacatctctattgaaacaagcgatgcgccgtaacaatatggaatttcagattgttaccccgtcttttgttaagaaacatttttgtggtaaaggaaatgccgacaaattggtcatgatagatcactttgagaagctgttcaacgttaagatgcgcggtataatggacatgttggatgtgaaggatccaaagcctatcgatgatcttgtagactcattcgcaaacatggtgtctggaccttacttcatagaaaattatcccgatttcaatagaggtgtaagaaatgattaattactggctgttggcagacatactgcttttcgccttgctgttgataacggcatttatttgggttaagggatttctgacattcctacactcgttgagtgccatgatatctttctatgctgtgtccccccatagtgacgtcaatgtacgagcagcaaatcaatctgacatgttggctgaatacgtcatgatgcgtgataaagcaactaggatcgtcctgttttctacactggcgggggcggttattatattcatacaacacgtcttggaggcaactcatgccgttttatgattatgcctgtacaggttgtggtaattctttcagtaccagaaggtcttgtgctgaacgccatacgcctgaattagatccatgcgctgaatgtggcggtgaaatcaaaatggtcattggagcaccaaagatcgtatctggtgtccgtggcccacaatccgcacctgatggcttcaaagacgtattgcgtcatattaagaagcagtctgggaaggggaatacaatcgatgtctgaccaacaaatagcacaagtcctcacacctgattcattctcagaattggtgttgatgcgagcgagccaacgcaaggagtctattcttgaaacgatggccagtgtttgtgaagagtacgacatcgaagaggcgaaagttaagaaactgattacaccacctctcctgtcaaggctaacagctgaatgttctgatgctcggttgttgaagggtgaattgaaatctaaaaaactcatttaaggttgtaacatgagcaaagagcaaatttataaattgttgaaggcgcaagagtatctgccaggttctattcgctggcgtcatggtagcctcaacgaacatgctgatgatatggatcgtgtacgttacacgacgccggaaggtaaatcctatgtcatcgaataccatacattcttggaaggacacaaaaccttctcagatgtgtacgacattattgaaatagacccagcaaaacaaatgattgcgggttaactcaatagcaaaacgcagtataattacaccgtcggaataattgacaaatacatttacagaatagcccgaaggggcggattatagaggaatattaaaatgggtaatttatttgatcgtcttaaacaatctcgtggccaacaagccgaagctatgcaacagcgactcgctcagcaaggccagcgtgttggtggcggtcgtgacccgcgcatctggaagtggacctggaatgacaagggtacttctgaaaacatcattcgtttcctgccgatccctttggtggacatgaaggctcaagaagaaggcaccatccctgaagatgctgtgttaactccgtgtgccatgatcatgaaacacgcattccagggcgctggtggttggtatatcgaaaactctccacagactttcggtaacgatgatcctgttcgtgaccatgaccgtcctctgtgggcacaacagaaagaaacgaatgatgaaaagctgaaaacagttctcaaaaaacgtctgccggacaccaaatactacgccaacatcctggtgatcaaagatggcaacaatccggaaaacaacggcaaggtcttcttgcttgagttcggtaatgccgtcaagaaaatcctggattcggctcagaatcctaagttctcaactgaccctaaattcgacccgttcgatatgtgggaaggcgcgaacctgattctgaacctctttggcgaagagaaagagttcggcaactggaaaggtctggtggccaacttcaccaatgtgaagtgggatactccggcacctttggggacagacgaatacattgaagagatctgggaaaaagagcacagcttgtttgagttctttaatccggccaacttcaaatcgtacgaagatctagaaaaacgtttgcgcaaagttctggccatccctgataatcagcctctggttgaaggtggtgcttctactatggcgcatgcgcctacccagtctcaggaaccccagcgcccaacagcgcaggaaagtctgaaccaacaacaatctcagccttctaatgcacagcaatctgttccagccaataatggtggtgccgatgcgaaacagacggcttccattgatgagttcgagcagttcctgaagcaagactaatttcgctaaagccccttcgggggctttttagtttttgacaacatcctgcaacaatccgagtaaactactcttctcagacgccgtcaggttatcattgcctaatatatccctctggatgccgatagagatcctttcgaaatcagccaggctgataccagccagattgtttacaagctgatcgagttgcttataagccagcagggcttcacctgtgaagtttccttctttgatctttttatacagctgtccggcttttgacccagcggtttcccagtctccggaagcaagagcatcagtaatacccaaaggaagaaaatcgctggcgaggctgcgcgtttcatattcagtctccgataatactttgttataagaaaaggaaatgttgtactgattaaattgatcggcggcgcttttatccagatctatagaactgaagttgatggggtgagcctcagtcacataaacgcgatgaacaacctgatcttctgtatccatttgctctatacaaatatcggtcacgaaatcttcataataacccatcttggttgtgtatgggtcgaatatcagattcttccatttgtccatgaccgacttttcataataatcgttggcgaggaggaacgacaactccagatcaatgttcgtcttgttgttcggcattttaatgtggttgccgttgttggtcatgggagtagtgtcaatccctgtaccaggtaaagatgcgaccatacacatcatttgcagggaacgagatgtttggtttgtccctccaaagaatgcgtttacaatacgggcgctttgtttaaataaatcgccgaatgaagatgaaggatacgcgttcccatcatttgctagtgttgcattggaatcaaatatcccaggcggcaatggaattgtaacacgaaatctgttcttgcgggaaataccccgttgaagcagttgcgttagaaaattgcgatagtcttccacgatgaggactccatttaaataatatatctgttgatatttataaggagttgacaaatggctaagaacgctatgggtgaagaagacccgttgttgcttcccgccgagatggacgccccagaactggtcaagcgttacatccgcaaatatcgtcaacattttgggccggaagcgaaacgtaatatccgtcgttctcatgtgtggtttatggagcgcgtatcgaaagatgctaacttatctccgaatcacatgatgaaagcattcgctgaaaataaacgtcctgttcagggtgtgcgttatattgttggtcgcatgtattatttcaaatatgatgcgctgactaaagatgaactcccgtattgggatatgtatcctctggtgttcttcttcaattttgtgaagggggatggggtgaaattcggtgaacgtggtgtgacctatctttatggtctcaaccttcattatttgccaccaaagctcagattattagtgtttgaagatcttatcaagttgaggaacgaaagagcgtaccgttctaaaactcgtttgagacttacctgggatgccctgaaacggttcgccaatcaccctctgtacaaccattgtgtgaaactgtaccgcgctgatcaatttcgttcacagctttatgagattgagcctcaatactgggaagttgttttgtttatgcgcactgctcgattccagaaacagagccagatgtctgtttggaaggacgcacgtcgtaaacacaaaaatggttagttctaaaagccgttctacgtgataattcgcgaacggctttctataaacatgaggttatgatatgtcacagctacatcgtatttttggtattgaattcagttctaattgcgttcgctatgatcgcacccataaagattcgggtggtaaaaccggatatgaactcggtagccagttgggatattccaagggacaattgtttaatcagcacaggctgaatgaaacggatcttcttaaggttatgggtattgaacttgattccgaagccagcttggatctgggcgacgaaattcgttttggcaaaccagaagcattggaattcattgaagacttcgtttctggttatacccaagccgctacgaaccgccgcgctgctaatgatgcagtgggtaaattctactttgcggcgaaatatcttgccgacaaacgtatacagatggaagcggaattcagtaaagaaaatctgaaagcggttttcggcgaagaaggcgatttccgtttaatgccagaatctggcactgattcagctcgtgatttctttgaaaaaccatttgtggtcaaaggctgtaaaattcttaccgacgaagaccacgctttcgcaatccatactgaagtcgtttacaatgaagagaattgccttgttatttccagattgttcaacagtctgtcatatacagcgtattctttgattgaacgttgtatggaagaagagcatcgtcaggtggtggtgaacaaaattcgcttacacgttgagcagaattttaacgggtttggttgtgaatccggcgaaatccctaatttctatctgtagcacaagcagggggctgtatgaagaaatatgttcgcctgaacacggtactggaatctgtaatcgaatatttcataatgcagcacctcagagcagaaaatgatcatgtgagcgatgctttgacttatgaccaccggatagtggttcattccgtcgaaatagtggatgacgaattgttgattgccaatgttgagcatgctctctgggatgaagtgttagatcagtatgttcaaccgaagttcacacagatcgagttgtctgccatgtggccacctgaaacagttttcaatattgatatacaaagttaatattcactccagtccgcgctgatacagcgttccgttgtctaacacagaagtgttaaccaacttttcaataaaaagtggtttacatcttctaaaaaggcagtatattaatgttccagacgcacagttgaacattaaactgccaaagaaggttactacacaaaagggtttttattatgtcctatattcttcatatcgagtccggcctgaaatttgaaattgatggtaaaaccgctgctgatcttcaagcagaaatcaaaggtgcagaccttatcattggcaatgtaacattacgccgcatgatggaaggcgtccttcagtcagccaatggctttgaactcatcgaaggtctgagccaggaagagaaagaagaaactctcgaagctcttgaccgcgctgaagccgaaaaagttaaaccgaccgaagatgcgggggttaaacagggcgacggtgaatcaaatccggtgattgacgccgaagaaacggttgggcaacctgtcggtgatcaaaattcccagaccgtcgaagacggttctcttgaagtcaacaaagacggttccatttccctggtagttgaaggcgaagaagttcttgacgaagaagcgcaagcccgtgctgaagaagttcgccgtcgtatgctgggcacaaccgttgctgaagcaatcgccgacgttgaaaatccgaaggatgatcaggctttagcgaacgcgaaggcggttctggccaagaaatcttctgaaatgcgccctgcatcttctaccgaagaacgcaagcgtcgtcataacaagcgtgaagaaatggttgaagcggctcgcgcatccaattatggtcctatcctggctgcggttgaatcgggggttgtccctggggtttatttgagctatgtgaatccggatatgcgctggttccagttccctgttaccgaactggcagatgaagccaatccacacgcccgcaccaatacttatgttgatctggcgccgatcgtttccggcggctggggtttcagtctgtatgtgaatggtaaatcattcaccaaacgccagaaaatcaaagaaactgatgccgaatctctggtgaaagcaatcaatgaatggttgcctcaggcgttggccgaagcgaaagctgctgcgtaatttaattcagaagcgttcaatagataaaggggctgctataatagcagctccttttgtttattggagcatgtcatgtcttattcgctcaaagggctgttgaagcgccctgtgcatttgtttgtcaaaccacctgctgtagaaggggaatattcggcacgaggagagttgtattacattaaaggctccaatggtagtggtaagtctactgtgccttcttatttggcggagaatgatcctcaggcgtatgtcgtaacccataacagtaagatcatgctcacggtttgtccatcttacaatattgtctgtgtcggtaaatatgacaagtctaagtccaaaggggttgattctctgaaggatactgagcagatgttgttcgcgttgtctattgctgaccaaccagaatacctgaaatatgatgtgattttcgaaggcatcattccgtctacattattgagttcttggattccccgcctgacacgcccaccacgtgaattggtcgtcctctttatggatacccctcttgaaacctgtattgcccgtgtcaaatcgcgtaacggtggcgcagacttcaatgaaagtctggtggtcgagaagtgggagagagttcatgatcaccgtcaacggcataaaggcttgttccctaccgttcctgcaggtatgatgaagtccgatggcctgactgtagagcaggctgtttttgcgtttctcaatcgtgattttgggagtatttattgatggaaatcaaagcaattaacaacaacgacatgctgaaacaggctgtcttggctatccgtgagcacgggattgaatctgaccctggtaacgcggagatcaacacagacggcactcgttttattgatggcgtgacgatcaccgtctctgacatccgcgaccgttggctgtctgttgaaggacgcaactcatcggctattgctgccatcggcgagaccttctgggttctgtctgggcgcgatgatgtgcgcttcttgtcccgtgtcctccctcgcgccgccaacttctctgataatggccatacatggcgagccgcttatggtccgcgtctgtacgcccatggccagctggatagcgttatcaaccgcctacgcaagaacccaaatactcgtcaagcgtacctcaccatctatgatccggctctggattcagatgctggtctggcgaagttcagcgagagcggcgaagcaaagactaaggacatggtgtgtaacctggctctgttgttcgccatcgttgaaggtcgcctgaacctgactgtcatcaatcgctcacaggatgttctgtggggtatgagttcaatcaacttcattgagttctctattcttcaggaagtgattgctcaggtgttagatgttgatgtcggtcagtacaagctattctctaacaatcttcattactacaacaatgaagtcagccagaagcagcttggcaagatcaccaaagacacaaaagtcactggtggtttcatcaactccatgatctacttcacgaacgtgaccaaccagaaccatatccgcaatctgttcactggtgtgcttcatcactgtgatatcggtagcccgtgggaaactgttgttgctcacctgaaggagtacggcgcggatcgcggcctgatcgtgcagatggcgtactgcctgtactgcaaactgaacgacaagctcatcaacatgaacctgattcaggatcatggtttgaacatcgcgctcgcccattctccggttgaccgcaagatcgttgatcccaagttcttggaaggtgcagtatgatgtatccgaaccttgaattctttacggggcgcaagcgctccggtaaggacttctgtttggagtccctcatcagctatcaccatttacagggtgatatggacatccagcgcttatctttctcggacgagttgcgtcgggtggctcattacatctattcttggttgccagaggaagtagaggacgatatcaaggacttgccatatgttcacccagataaccccaagggattaaccccaaggcagatctggcttcatctcggcagcgatacggggcttcgttatgtccaaccggatctgtttttgtctttctttaagcgttaccaactcccattagttgaacagaatcctaacattcattatattgtgagtgatctacggacgcctcaagaatatgaatgggcgctgagcactaaatgtcccatcacccgcatttcaaaggcagatcgcggcggtatcatagaggacgacatagaggctttcattgatgagatgaaagttgattatgaatttgttaacccatttgaaggttggcaaccattcgtcaaattttatagggaacggaaatgatcacagcagagcatatcaaaagtctgctcgaacttcaaaaggccaccaatgtggcctattttggggaagagtggcgtaaacattggactcatgtggcggtggtgaactccatctatcgtgagtgggcagagtttcttgacgaaacaacccaggactggaaaatctatggcggtgggatacaatatgaccgagaagcagctgtctacgaattggtggacgtggtacattttatgttgtcttccattttatctggatttggttctgctttcccagttgaaatgcagattgaccttttcttcaaggacggacgagtgatagacaaatcggccaatctggaatcggtcacagaatgttttacgtcatttatggaacaaccagatgttatccgttttgtgcggtttttatcaaaggcttgtggttatttggatctggacaccgaaacctacatgctggcccacaagcgcaagaacgatcgcaatcgtctacgtgctgctggcggtgcgaactatgataagtccgcggaaacccctctgactctggagttctaatgtttacagtcgtcgtatcatattatgcggaacatgccccgtacatggcagagaacttcgctgagtttattaaacctggcagacatgtcgttatcgcgaccaaattcaacaaagaaccagatggtgcggctactgtaacgcaacatattacaacaccagagccggaaacactttttaaacgcattgaagattttgcttttggcggggaagaaattccagagtcattacagcatatctattttgaagatgcgatatattatgttcatgctattccaaaggatgttacaactgaacaaatgatgtatatcctgaatatcgcattgaatgtttcttgtgtcaacagggcacagcacaatcgtctgcgtaatatgtatttaacagaacgcgggggttccgaaattaacgatgaaaaactcccgtcaataatggcgattcacgaagcaatcaatatattgtccaagaaagtcttgggcaccgatttattaattacgatcaacaagcaagaggaaacgaaatggccgattcattgatggctcgcatgctcaagacagcaaagaaactggacccgaatgccgaagtgctgtcaaagaccgatgcgctgaagcctgacattatttgcagtacgggtattcctattctgaatttggcctggtccggtcgtattgatggtggtctgatatcaggcatcaaacagttggtgggggattctcgtacattcaaaaccatgtttgggctggtggatgttaaggcttacatggataagtttcctgatgcaatttgtatcttcgcggattccgaaggcggtgcgaatgaaaactactggacatctatgggtattgacatggaccgtgtcttgtatctgccgattgaaaacgttgaaaagacgaagatcaagctgacacagcttctgaatgatgcacagaaaggtgacaagatcatcgtattcatcgactcaatcagccagttgccgtctaccaaagaagttgatgatgccatagcgggcaaagacacacaagatatgacccgtgctcgcgctctcaatagtttttggcgtgttatcactccgttggtcactgaaaagaaattcgttttggtatggatcaactcgtactatgatgaaatcgggaaccaatatgccgagccgaacatcaaaggcggtaaacaggggttcctgtcttctaaccaattgtggttcatcacgcgttctcaagtaaaagaagacaaagaccttttgggatggcagtttacagtcaacattatgaaaggccgctttgtccgtgaaaaagccaagttccctgtcactgtgttgtatgaaggtggtattgaccgttggtccggtatgttagaaattgcgcgcatgctggggtatgtggatttggtgagtggttcttggtatcaacgcactgccaaaggcgggtttgatccagaaaaagaaaagaaatatcgtaaggcagagctgggagatgacttctggtatccgctgctggaaaacccagactttgttgacgatgtgaacaacatgttcggtatctctcagagttcggttatgcctgcagatatgctggagcgcctcgataatgtcatcaaaacaaccgagtaatattgcagggggaggagactcccccgttaattacaaaatcatcgaccctggttctgatcagttagcaataatcgaaataacggaaggcaaattccgtggcgttcaatttcgtatagggaaagtgggtgtccatttagacaatggagagcctcggttatcctttacaacagatatattgaagaaaccatggcgtttgttatttgttaatttgaaagaaaatgacttgttcactgtggtgtctggggatatcttagttgatctgatacagcaaaatgctcaggattacaacaaaattttggtggggtagttgccaatgttactcgaatctgtcgtgctttcccaattaatctataacgaagaatatcaaagaaagatccagccgtatttgaaagccgattatttcgataacgaaggcgagaaaattatattcggtctcattgaccattacacttgcgaatataatgctcgtccttcggttgaagcgttgtctattatgctggaaaagacttcgctcaacgaacatgtatttgaacaagctatttctgctcttgagaatatcaatgacaacacattccatcaggaatggcttgtaaaagaaacagaaagttgggcgcggcagaaagctgttcataatgcgatcaaacacgccgtcaacatctatggtgatgagaaacgtaaagatgagatgaacacgattccaactctcctacaagaggcgttggcgataagttttgattcttatcttggccatatctattgggaaatggctgaacaacaatacgaccacatgaactctaatgaagcgaagattcctttcgctgtagagatattcaacaaagcgactcgtggtggtgttggtaagaaaacgctgaacatcgtgacgggtgcaattaatgcgggtaagacaacaactctgattgatttggctgctggttactccgagcaagggttgaacgtattcgtattcaccctagaagtggctgagaacgtctggcgtcaccgccttgatgcccgtatgatgcgcagggacttcgagtccttagagaagctctcacgacatgaatatgtcgctacgatacaaaagttgcgaactcgtcaagacggttccatgaagggtgatattgttatcaaggaatatccttcgggcgcggggcatacaggtttgtatcgtcgtgatattcttgattatgcgactgcaacaggaattacacccgatgtcatcattattgactacttgggggaatctgcatcttctcgccttcctgcccatttaatgcagaacaccaacgtgtattatacgtcggtggcgcgagaattccgtgcgcttggatttgaatttgattgccctgtatggacaggtatgcagtttaaccgtgaaaaacaatcggcgactgatggtgatattagcgacctggcagatgctatcggtattccgaaggttgcagacttcatcatggcgttctatgcccctgatgaactggcggctgttaagaaagcccgagcgtctatcttgaaaaaccgttatgccaataaacagaaactcaaatcgttcttgttcggtatggatcaagataaacagatattgtttgacttggattggaatgaagtcaagcgagacctgacagaagaagaagcccgttatgttgagaatgttcatatcaaacatgacttaaacaaaacaggcgacgtaaatgatgtcaagaaagccgagaccgtgaacaattggaatttcggttaacttcaataatggtgatcatagggtataatctctatgatctgaatattaacccaggagcacatcatgtcggatgttgcatttatagctgacctgattcgggcggcacaacaggtgtgccagtctggagaaaccagtctcaaactcactacagagcagactctagaattttataagaatcgtcggcattggggtaagaatgtcaagatacattgcgaaccaggtgaattggttcagcttgatcttccgtcttttccattgccagtacagaccaacactcaggaatattacaatgttgaaggcgttgatattattcctaagtttggttttcacagtatcgtttacttcttcctgcgccgtttgggaaccagtatcggtccactcggttctcgtcattaatgaggatatattatgtcagttgaacaaattggtttttatcaactcccttctgatccaaaactgcgtcaaaagatgatgcagactttagaaaattgtcgcgccgcccagatccgtatcaagtctgaacagacatttgttaccgaagccctggctgaattggcaaaagaaacaggcatcaaagccgttgatctgcgcaaagtcgttaccgatcgcgctggtggaacttataccaagaccatcgagaccagcgccaaataccaagatctttatgagtcgctgtatcctaacgccgcgcctgatcgcactgctgaataatatcggtaaaagcccaacatttgttgggctttacttcaataaaactttacttcaataaaacttgcggctatcatttaacctacattgaaaagtttcactacatcatgagaggcaaaaatgaaagccaagaccttcagcatcccaattgataatattggtcgtgtcaaggaacgtcttgccaaacttgaaagaacggcaaaccgcctgaaactcgagtttcctcttgttgagttcagtgaaccatacaaaacacaacaccgcgattctatcactggcgaaaaattttatcgttggtggcaagattgtactctaactggtgaaggcatcgaccgccccgtttcttatggtgggtggagtatcattggacaattcaaccaccagtatccaaaagtcattctgaataaactgtcagatgacatccacccaaatttcgttcaaagattcgaagctgaaaacgtatcttggtgcgagcattgcaacaaatctgtgcgtcgtcataacacatatgttgttcgtaacgaacaatctggcgcacagatgctcgtgggtagcagctgcatgcatcattatgtcccgcatcagaaatcccttgatgccgtgatgtcttattatatgtcgatacatgaaatgttcttacctgatgaagatgaccctgaaggcatctatcgagtgaacgaacctgattacgttgacactgaaggatatttgtgtaaatgctttcaggttttgctgtcgggtatgagtatgaagagcgatgattttggtcgagttcttggtcatatttctagcgggactcgtccggagaaaggttctgacattgagatattctataacaaggctgttaaagcccgtgaagatgctcaatcagagatgtaccatatgatgcttttcatcgccgcgttgtctgagaacaatgatttcaacgtccggctgaaacgtatgtgcgaacctggttatcaccttgttaaagactccacaacagttcgttggggagcagcaaagtattatgattatattcacaccccgcgccaaacgcgcactgtatcaaattgggttggtgaagttggtgaaatgttagaagtacaggtcaaatttgaagcaaggattttcctgtattcgtctgattatggtgacacgtatctgtatactttcaaaaccaaggaaggcaataccattacatggaagacttcctatatggaaaccgagtttttagaaggcgatatgatcattcgaggtcgtgtcaaagaattgaccgaattcaaaggtattaaacaaactcagattacaagggcaaaactgaggaaattatgattcttttacaatttgttttgaaagcatttgcattcggtcttatcgcatgcggagtgatcacagctatgctgttaatcatagccctgatcatcccatttaagaggaaacgttaatgaaacgggaaatcttggatgtttactaccctggatcttgttgtccaggccatgacaagtacccagcggacacatacaaaaatcgccgctctaaatcaaaacgttcagacggtattaaaaaggagcatcgacatgcccgccgtgtcaagcgccaaaatctgaaatctgatttgctcttcattaccaaggaggatttctaatggaatgttcatgcggagggcaaggcacaagtgcatacttgccatttaacaccgtgaaggaagccgaagaagccggatacgccgtggataaagctccctgtgtaatcgccaccaaggattgtccgtgctgtaagaggcactctcaggacgtgcattactcaccaaacgcacgttcggataaagtcaatcttctttcactgatgagtaaatgacatgcgcaaatattttaactggttgttggggaaacccctaagcgtcgaaattcgttgtcttattgatgctatcgacgctgatgacgttgaatggaatggtgaacgctttgtcggtatcgatgctgtcatctataacggcgtaagggtatgtgcatatacagtcaatagcaacgcagggtatagagtcgatatcggtttttgttacgattgtctgccataccatgaggccaaggccgtgtataaagcgctcacccgacaccacaggcgtcggattaatcgtaaagcaattcaggtcgatacagatgaccgtgaaaaagtccgcaaggctcttggctatcacaacaaataattcaataaacgtttacccatataatacatgggaattaaacagaaggaattaaacaatgtccacactcgtagaaacattattcccaggttgtacgcttgggcgcggcggctctgaggccaatggattagcgattgatgttgagcctaaaaactgcaccatcatcgacaagtttcacgacaatcattatgctctgaatgatcgtcgcgatcattatttggtttctgtgggagaatctttggttcaccttcgaatctgttctgctttaccagggtatgggtatgacgcatttgcttatatccatgctgtttccaacggcgagatggggaaagaatccctgattgaaaaattcaagagttggaaggaatccttcacctctgtcacaccacctaagaaataattgaggaatatattaatgtcactgaaaacctttttaaaccgcctgttcagcactgtcgaggccaatttaactgcaggtttggataaactcacagatactgagaagcgtttgaatctggctgcacggaaactatctgatgaaatccggcgtttagaagaagcccgtgtaaccaatgtccgccatggcgttaagatgaagaagaccgctgatgaaaatctggctgaagccatcaagcgagaaaccattttgaaagatgcgatcgctcgcggagtaactcctacccgcgccgaagccttgatcatactccaccgtcgtcgtattggtgaagctcttctggataaagtcaaagagatcgaatctggcagtgtgaaattgaattcggcaattgttgaattaggtgacaaactggacgctgttaagtctaatctcgaattggtccgaattcagaaagaatcaaatgatcttgggttaactttaccggaagacatcgattacgcggctggaatgattaatgtagatgttgatgctatcctgcgggaagctgaaatcgctgattccaattattctaccaatagcccttcagcaacagaagcggacaattatctggcaagtttatcaaaataatatagaaggggcatatgccccttcatttgggggatttatggtcattattgactcaacagatattttgattgctgttcttagtatggttggggtctgtattctcgttatgggaatcaaaaaatctttgggaaaacgggttgttgaggccgtctcctcagatttacaaccagaaaataccacgaaaaatattaaagacataaaacttattctgagtagtcgtgtgtccaaaagtttggttcctagcatagatgatttcaacttagaccacaaagacccagacactactcctgaagaatattttcaatatttcatgaaaggtttgggttggccgttcatagaatcgtgtgtatcaaaatcaaattgtttctatttgaaaatagcatgtaaagatggatatcaggctgctgtatggttttggaattacaacgacacttacgataaacaatttttgatatatcttcacaggttgtttaaagagtatatgaacaatggaaatgtccctattcacaaggtagaaactgtagaatctatcgtggatcgtgctcgaactcgtaatggaacgaaaccagattatctttccaagtatggatttgtttggaaggaagacatatgaaagatatttcattttatatttggtctgcaatattatttttcgggatcttgattattctttttcaaccggtttgggccgaatggatcaataatagaagaatgcgcaaatatggagtggatgttcttcagattgttgacaaggccagaaaaagacgcagcaaataaatctattgaagtctctagggttataatagcatgacgaataaccctggagacacatgatgcttcctcttttagaaataattcaaaatcttcgcgataccaaaggcaccaacgccaagaaagctgtgctgaccgaggcgttcagaaacaacccagaattggttgatttccttcaatacgtctacgacccgatgcgttcttattaccgaactcaattcaatctaaacgcattccctcgcatgctctcgcgcggcgttgttggtagctgggatcaggtttatgatgttctcgatatgatggccgaacgcaagatcagtgggatgaaggcagatcaagaacttgcgaaggctgcaaccaacatccatccggattaccataccctgatacaaattatactcgacagagacatcaaagcgggaattgccgagaaaggtataaacgcagcattcaacgcagcaggtggtacagggcgtctgattaatatccttccatatcaccgttatgataacatgacgattgacttgctgaagaagatggactttaagcgtggcgtcttcagccagctgaagtcagatggcatgttcgccaacatcatctgtcgttatggaagagacccagagattcgttctcgttccggttctcttattgcgggtggttccgttgataacctgtcattggttttcaaagacctgatctacgatgcggggattggtgaaagcgtcttccatggtgaactgctcgtcattgatctgaaaacgaatacagtgttgcctcgggctatcggcaatggtaagctcaacagtgtcatccaaacgggtgaacccctggaagatcgttacaaggtgatctatcgggtatgggacgtcgtgccgtatgataattggttcaatgctcagcgtgtggacaccccatatgagcgccgttttgatatcatacaacagttgttcgaaggggatgacggactggttcaggttcaagaaacccgtgttgttcattcatttgaagaagctgtcgaacacttcaaagacgctttggctcgtcgggaagaaggcacgatttgtaaggccgcagatatgccttgggaagatggaacatcatctgaagggttaaagctaaaaatggaagtagaatgtgaccttgaaatcgttggcttcaacgaggccgataagaaagggaagcacgccaagacatttggatctctcctctgcaagacctctgatggcctcttagttgttggcgtatcggggatctcagatgagttgagactacgaatgtgggaaaaccaagaggacttcattggtaagattgccgccgtgctctctaatggcgtacaggataaaaccgatgacgccatgaagtctctattccttccacgtctcgttgaagtacgcacagacaagaaagtcgctaatacactggctgaagtctatgctatccagaaagcatatatcgaaaatattttggtgttgttggaagcggcatgaaaattggggttcttcggaaccccattcaataaagacagtagcagtataattccaagaaatcattaccaggtgttattatgaaacttattgataagaaaccaaggaaagacaatgattctcttaatctcataaacattatgagggaattgggagtttctgaattacagatgatgaagatgatgaattgttctgatgtcgtttataattggaccaacggctatcgtctcataccaaaatcttttcggcgttttattctggcgttggtttttattcaccgacaagggaagacagaagagtttcaacgtttcgttcgcacagaggagagatcacgtggttggtaaaaatccgccgtcatatgccaagattggaattgcttttcgaaacgccattctctttataaaggagaaaggattgctgtctgagtactaccagttttgttcagaacgccgccgacttcaaagacagaaacgcaaggagaaactccatgataactgagtgggagaaaatgcaatatgaaagggcattcaatgtttattgcatctacatggcgatcaaattgcatttcaccacaaaagactttgattatggcctgtatggaccgatgaacaattacaagtttgaaacgttctattctaaacagggcgtggccaaacaatttgctaaacttgctcgtcggttcgaatcgtcccaaggtgaagtggtggaaaattatattattgccaactttgttaaatcaccaaagacgtgggtgacaacattacttacaagacaggcccaagaaaattataatgaatatcgccgcctgtatgacaacttctcatataattttcttgaacattttgagagatatatgatccctgagataaaagaaagaggcgtgaattttatccaatatatcaaaggaaatggtaatgggcatccacctttactgacagatattattgttaaacgctatccaatttggtttctcgttggattgaataaagtagtgggtttcatccatttatatgatacaatactcaaggatgacatctattggaactctgagtcttttttattaaagaaaaccaattcagttgttcctgatgaaaacacagaatacacaaaaggaaaactccgtgagctaatccttgcccacggaatttaattaccaatcccagagaatgcgccctgtatcagtacggttggacggctccaactgtgaactattgaagttgttcgtcgtactgactttcttcgagttgtccacattttgttgtactggaaccacaacactggctggcgggttagcataggcggatttaacttcttcgatgttattggctgctcttccttgaaccggataagccccgcccagagtacctcccccaatccctgcgtatgcgtctttcaattgtgaagttctatccgtaacgttgacagcgtctgaggacaccccagaaggcatcggagttaacgtgttgatattggcattgggttgtgctggtgattgactgctgggcggcgtagacgcattctcaggagccttatcgtcttggctgattagcatagactgtcttgcttccgccattttgttcctggctgcttcaccacccagagcatcaggtatgatactcacgaatttatccacgatatcaaacatggcattggttatggcattcaacatattaaagaatggtttcttcacgttttccgcaaatcctttcttcatatcttcaacgactttatttgcagccgccatgccatcatcaatcaatcctgatatcccgcccgttacccaatcaaccaattttatcaattggccttgtaagttgtctgcaagtttaccagggatttcagtcagcgctgtccctttaccgatcccagcaaacagatcgttggatacccaatcaacgatagaattgagccagcgcactggtgcttcggtcatcttcaatacttgttcacggaacgcctttccaaatcctgcgttatcccaaccaaatatttcagccacccaatcagccagatctccgaaactgccaacaagttctgttatacccgcctgaaccctgtctactattgaaacttgggctttgccaaggatttctttagcattgaagaaacctttaccgaattcaaatacagatgttatcagagccaaaggtccgactcgaagcattttggtcgctgctttcaaaggcttcaatagaccaccgatgcccatcccgaatcccatgaataacttgatgaatcctccaatgagttttagtggagacatcaggagtttcaatgctcccaagccgagaagtgcacccagcgccgcgcccataccggaagattgcccgtcctgttttggttcagaaggtggaatagaacctgcttcgggattgttcttgttctctttaccttcttcgccgcgccggaatttctcttcttctcgatatttttgatcactctcatagatacgcgttaatgtgtcattgaggtcttcattcatgtaccccaatttatcatcaatgctgtcaagacgagtaacggtttcgttgttggcatctttattgaagtccagcgtgtccttgtgggtctggagcattatgtcggtcaggtttacaatcttccctcccaccagtgctagtttgtcctgtaatccatcaagacgttctagactatcttcattcgctgctcctaattgatccgatatcatgtcaagatattcaacagacatatcatcaacaggaccattcgccgcttcgccaaatttttctattttggttgcgatccgcatcagttcttcatttccgtccttggaaaggctgacagtgtctttcatccacgtgctatagtcccttgtgaaaataggactaacgcgaacgaacccaccaacgatgtcattcaccgatgggaatttgattggagggactttctgctcctgattagacgcctgtcttgtcttcagagattgcagttgtttgttgacatcagcgagctgtttattcgtctcggattgggcgcgcaattgcttgcgctccatcatttcctttttgattttatccaagaccttgacgatgtcctgctgccctggtgtcggttgattgttatctgccatttgtcacctctcagaccttctgggcttgtcctttctctgttggcgtcggggttgggttagaagccacagtttgtccagtctgaagtggtaataccttctggatcatactgtctttggcaatctcagcgacgcattgataatcatttgtcaggcgtgaaatcttatggcgtatctttgtgacaatatagtggcctgatgataacttacttaactcggcaatgttttcattagatttagggcggttagacaagtcttcaatataataaacctgaccaacgttcaggcggttatcgccgaccacgagcaaacgcataaccgtagatgctagactgaattctatgacgcggcgagcataatctatttgttcagccccatctggttgtgcctcgatataacgaacattttcacgatcaaattgatccgagaataacgggaacgggtcaaggtgagccgtgctgtcaaaccattctgaataaatgcgttgggtcgctgtcgtggattttgtactaaagtcatacacgcgctcgttcactgccaagatatcccgataattcgccatgtactgatcacgcgccagtttcttttcagccttgatgatagtccgcatgaaacgttctgagttgaaattgttatcctgcatcaacggcgcgtcttgagggtctcggaagaatttcttttcaatagaagcctcttgcttctcttcagcagtcatagactggttgccctggttgaacaacgtcgtcatgcttttgaagtgataacccgtaaagtcttcatagaaaacgaacggcatgaataattcgtcatatgcacggctggccatataatctatggagcggagtacaggccaaagaggggtagcgaacctttcttgtatcccatatgaaggatctatgtcttcaaatttggtgcgtgaattcagttgctcaaagatcttagctgccatctcagaataagaaccgcttaatccaagagatttacgcaacatgctatctcgatacgcgtctgttgttaccaagtgtaaccagaatgcctttttcgaagaagagttcgattcatcagcaactctccctacccgactgactcgcaatgaaagcgtagtgtaatcagaagctgcaggtgatttgaacgataccactacttcctcacctccgaggattggcattgtatccaatatatcccagccttctttgatcagaatattagctgtcagcgacggtgaagcattcccttccagaccaagatcctgatatacgttgaattcttgaaacaacgatgaaaggtcataaggcttgggagtaccgccttcaggcgtgtgcggtaatatcgccatatatttcaaatcaaatgttgtggacggagttaaaattccgtcctgggattctttattttcgatcatttggtcagctcctgttccagctgattaacaaaggaagaaacataatctgggtctagaacctgaatattccgtttggcttcgtttttgtttatagcatcatcatgatatgtaatacctgtcagaccatagttggcgataatcgtggaatcatccatagaaccaaggccatatgctaaacgtattgctctaggatctgtttcatgaccgaattcgtcaacgtagtatttcacatcccacattccgtcaaggccgtatcgagccgtcagttcttcaatgatacggcgctctggttttggccagtcctctgtgatgtccatgataccattgataagacatgggatccaaaacagctcgaaggaaccgtagacgcgttcggcaaaagaccttggggtttccccatcaaaaacagtatacggcaggagaagcccttctatgtccctaattttcttaacaaccataacccgtcgtgttaagttctgcaacaggacttggtcatcctctttgacaccaattagttgatgccacacgagtggaaatttctcaaaatatttcatggcatcttctccttaaaagctgtcgccttcggtatcaaagcggcctttgtgcaacggttctagttctataaacgtcatgtcgatttgtgtcgacacgatgctgtcatctttgtgtacggcatatgaagagtcaggcgtctcgttgacaaacatatttgataggacgcaagtcgatatccgatgcaaccatttgttgcgttctccctggaccatgaacgtgatatcaaacgtggatggatgaagatagaatgcgctggaattcttgttgtatttgtattctggatacatgtgcatcttgaataaacggataatctcccgcaccattttcgcctctttttgcgatcttggagtgaatttgaatgtgaacgggatttctcggttgcgcaccccttggaaaatcatttccacatacgggttggtcatcgtacctgtaaacaattctaatgtatcatgagcattaattgtcggcaagaaaggaattgcttccgaagcagattgaatggcctttgttgccgcaaatctccccatttctttcccaacattcaaagcatccccaagtttgaattgactcatatcttgggcggcgcgtgataagaattgggcacccatacctgctaatcccaactcggagccgttccagccaacgccatagttggttgtaatggattcgggcatacacaacacaattgactcgttagagcggacgtgacgcgcccaagcatatttgctaatagaacccgatttagaaccataaaccacaggagtcttacccaatggattttgtatcggattttcgacggtttgggttgtggtgtccccatatgaagaaccagatattcggttgatgttgaatagaacataatggccgagggttttaccccctgttatgtctaatggatagaccaattgcttctgagacttggccaagccttttgtgttcagaactttgatcttatcgatggtcgacttgaaattcgccatggtctgaagccctttaattagaatagagtaatgacactatttaggactagaatatggctatgtatctcgtataccaaattaccaatctgataaacaataaaatatatgttggggttcataaaggaactccagatgatggttatatgggttctggccgagttataacaagagcgatattgaaatatggtgttgagaatttccgcaaagatatattgaaagtttgtaaaacatcagaagaaatgtatgaagaagaattcaggatagtgaatcaagaatttatagatagagatgacacatataatttaacatgcggtgggcgcggttcatttacgcacataaattcggatccattaatcagggccaacgcttcggagaaggctctcaaaaccctaagtttaaaatcaaaagacgaattacaaagaatttacaaatctaggggatttcctggagaaagaaatttttggtttggtaaaaacaggtccggtgaaaataacccaagatttggctgtgtaatggaacaagaaacaaaggacaaaataagaaattccaacaagaaaagagcggaatccggcctcgtggattattctaattgcaaagggccaatcacaaaagaagggaagaacgcaatatcccaagcgaactctagagaattcaaatttgttaatccagaaggaaatttggtcacagttatcaacctttccaaattctgtaaagaaaatgagttgagcgaaggatctatgagacacgtccataacggccgaaacatacaacataaaggttggaggaaagcataatggcatcgtatttacaaggaaaatttattcccaacaatcctcaaaaatatgtcggagacgtaaaagatatcgtgtttcgttcctctttagaattggttgcattcaaattctgtgacatgaatcctgctatcgtgaagtggggttcggaaacttgtgtaatcccttacatatctccggttgatggccgcgcccatcgttatttcatggatttgaaggtttggactcgtcggcaggattccgatgaattacaaattactcttattgaaataaaacccaaagaccagattaaagaacctcgcaaaactaaaacgatgaaggaatcaacattcaataattcaatgcgcacttggttggtaaatcaggcaaagtggacagcaactaaagaacattgcgcaaaggtaggttggaaattcattatctggacagaagaacatttggtgccaggtgaagacccagaagttaaaaaacaatttgctcttcgttctaagaaaaagcgcgaagtcgaaatggaagatcgacgccgcgctcaaaggattaatgcgttgaaagagcaaatgaagaaggaaactgctaataaacaaccaacgcaatcggaagatgatggtttgttattaccctgatttccttggatgtccaggggcaaagcaagtttgtttgtccctgcatatcatccacccatcagcacgggcttgtctagcacagtctccccaagattcaccagcataagacttcatcccacatctggggtgatgttcttcccaagcccaattacaagatttgcattcaacacactcacaatacaaatcgacggtgtatcctgcagtaatcatttagtcacccagatgatcataaattttttgacggaattcgttccctttagcacaacctgtagccaacaggcgagtaccgcccgtaccaataacactgatggtcgaaaacttcaggatacggcctgtaatagactggtccaccttaatggtttctactttaccaagacggagttcatcagcatcacgacgaataaatccccgtttgacaataacacgcttgttggtgactgcgaattcagttgtcaatacatttaaaattgttggtatcagaaataacaagctgaatccaaatgttgggataatcgttaaaataaccagaacataaatccaaaaaccgctccaccaagtcgggcgggtgaaagcgatgacatgttcattttcgccgagcatgcggtctacataacgcattttaattccttactttgtctgggttggataagatttgttcgtatccgacttcgccttgatcgtcgaaaacgtgtttaaggattgccatatctcccaccatccggtaaacttcaattccggagttcatcaatcttttgattccatccatgatacgatatggctcttgataatatactttatcgatcttgccattgtcgatgatcttctgagtgcaatcagggcatgggctatgtgtcacaaacataaccaaccctgtgaaatcatctgcattttcggggatacgcattagagcattttcctccgcatgaatgacacacgggtttgtaacaatttgcccgtgttgttccatctcacaaacattcggcatgccaggaggcgttccgttccatccgatagccacaggctgatcagtttcagggtttacgataacacaaccgacttgcagacgccgagcataactggttacaccatatgctgcagctgatcgcatatgggcgaacatcattcgtggtttgatagccattattttaccagagccttttcaagtaaaaagatcatgtcatcctggtcggcgcggccaaagatctgaccgagtgacaccatggaatgaacctctttgacaatcaggtctttgcctgttacaccgctgctgtccaattcgagaattgtagacatgcgctcaaccatatgaggtggatgattgatataacgttgaagatacttttcacgcaacgcaattttttcgataagatctgacatttctgcattctcttctgaatacagacgttctttagatttcagttcttcgaggcgttcgtacatttcttttaatgaaatcatgtttgtctctcccaataaattaaggggaagtataccttcccctttcttcaataacctaaaatattattcagtacatgctttaccgaggtctgtaagcctatagaatgttacttcagacttcggccaatgcttatactcttcagagttgtcatgaaccattgtaaacgcaggttctatcagacctttagacaacaaggattgacctgaacgcgtatcccatggatcaccctttacagaacgaaacccgccagatatcggtggcgcatgaaaagtccaaaccgttttccctttatcgttcgtccaagatttgggattgaaataccaatgacaaaggcgtctggcttcacctttttcaaaattcaacattcctaaaaggacgcagagttgatgtcctgtgatgcgtggctttttcataagatccccaagtcggtaaggtaacgataaaacaagattatagacagaaatccaattaacgcccaagacgacaggataggcttgcaatttagcgtagtcttcccattgggtatgttaggacttccggtaataacgacggagtctgttttggctctcagcgtcatgatagaacccatgagaatgattgggcgcacataatacccttcatgattgcgcttcacgatacatggtgaagaaaatcgcaggaacggatcaccgcttttataaccaaataactcgaccttttctatttcttcgggtactttctgaccaggaaagaatattttcataatacaattcctaaatttgtgtataacaactcagccagggttacaggacgcttgaattgttcccaactgcagttcaggtattcgcgctcttcgcgaatattttcgtgaagatgaccatggatattatacgcccaacggtctaattgataaggaacatgtgttgtcaacacacagccaacaggcgtgtcacgttcgtacatagcgccgaaggagctaatccagccgtctagatacagagacaacaacatcttaaagctgtcatggttgccttgtgccaccttgatgataaagttcggacgccagtcatcgggaacggggcgtcgcttaaactcatcaatgtttcgcttggcaccctcccgcatcaggcgaatgaacccttcagccccgatgaagcatatatcaccagccagttcaagaacgtcccgagacttcaacccctgaaagatactgtcaatgaccgcagcgtcatgggcttcctgtgtatcaaatccacgcggtttaaagaccttcttatgcccaagatgagtgtcaccaaggtgctttacaattcccgccattatggtttcctcagccattctaaaatcggtacgttcacaaaccctttatgccaacgatgcggtttgtgacctctattatgttcgagcaaacacgtgtctggtgggacactgaagtggatgccgtcgatgtcgtagtaatgtccatcaataagagtatacacatgaccttcaatctggctataatggatctctgcttgaggatacacatgcttcagaaccagagcaacctgatagcactttccgttcttcccaacatactcaacatccacctgttgtgggaagacgtctttgataagatttatcaatctcgtgatgtcatttacaacaaccgaacatttcatctctatgataactcatggcgatgtcgaacacaggcagggcaatcacaattctttttcaaaattggcaattcacaccatgtcattaagtcatgtattggggacatgtgatgatagaaccaagatttcttgagactaatcaatggatataaaatttcaatagatctttccccgtctctggtcatcatgttgtatccttcaataactttattaaaggcgggtatccagtgtattgcgtcatcgccgttaacatatgccaagatatacgtggacggcaagccactgcctttaaattgcgtgtttaataaccatatcggggcttgtgctaaacaagcgcgaaaaccaccgttcgggacgttgatactactgataggttcttgatgatgtgtccaacgatacatgagaagtggggcatttttattaatccagttgtgtattttattcctcgcccaattttcgcgcttagatttaaatttattattttcgaggtggcaatacacagtatgaactgaatatcctttcgataacaaccaaatcaacatgtatgtgctatccaccccaccagaccagaacaacgtcacttcttgattattctccggcaatggagcatccccttgaagaacacggtatacaaaatcacaatcagatactgagacaacaatttccattaaaatattcctcttaaagaatcatttatcctgcgcaattcgttttccacttttatcttcatatttttatcgtcgttcacgaaaatcattttatctaaatcttcgccattaatctttgtataaaaataaatgtcatgctcaaagaaaatcgtttttccaacagaacgcatggcaaagaacgttgcgatagttcttggtaaaggtttgaatttaatggcattgaaacgcaaagcctgatagagaggttttaatgttcctaagcgaatacaatgctggataatttcttgctctttcatttgttgtgtgccataaagatatgatggtacaatgataatacattatacaaatagaaaagcccctttcggggcttatttgtcttcagattcgtacaaacgccaattgccgatgctaaaccattcccctacattatttgaatatctggttgttcggactcctccgacttcatacgcccatttgaagaatttccatttggacgataaagaaggacattcgcttccggatttcaaagaacacgctgtgagccaacgcgtaatactgagagcctgaaaaatcggatggataaatgaaaaatacaatatccaaagcgttatgacaacaaaaacaatggtcgtgatcaccccgacaatttgaaagaacatttaagaacctccaaaatattgaacacagactatgaagaaaactatgattgtaatcaaccatgaaaacaaggaaagattcatccttccaccttcagccccatcaggtcttccatggttttctgaatgcgtagccagtcaagcgccgcatcataacgagggtcatgatagatcatgccttccagttcccacgccttgataccagcatatcgatcctgctgcccaacacccttcagccagcttacgatgtcaaatgtgtggtgatagtcccaaggttcgttggaatcttgcttacacgtcacctcaatgatgtgctgagccttgcggaggtcaaacagattcctatcacaccagtcaaccgagcgcgggtcgatcccaaggcgatggcatcccttgcgaatttcatcggccaattcgaaaatagaaatatccttgtcggtcggatatagactgatagctcgggcggcgtcgcacgggttgcgtttacctgtacccagccaccattccacagtaccctggtctttcacacggcctagttcaatctgttctttgacattcagtttaataaatgtcgtgcgctcttcaacaagctgctgaagggtataacgcttggtcaggtcggcgatagtctgtgcccaagacaacatgacagcatcatcccaacgaccgagagtttcgctatcagcgacggccaagaaattagatttatacatcagtttcccctgctttctctaaaaggagtttcaaaggatatgtcccacaagcggtggcatactcgactttgaaatatgaattatcgttcatggcgaaattgcgatgtgggaaatattccccgtcatccaacccgcctaatcccccgccgacgcgatattgtttcaactcgacttcgccgnnngccgccataacggagttgggcatataggccgataggagcgcgaacataatgctccagccatacccaatttccatcgtgtaaacgtgtagggataaatgcaaaacgcaccttgggatgaagaacggctttggcgagcatctcttctgttatttcacgcttcatccaataccttcctcttcgatgatatcttcttcactagagacgacagagattccgtagtccaccatctgggaaagttcttgtaaatcttgttcaaacccgtcaccctgtcggatataaaagacgggtacgacacgacgcctggtatcttcgaagatagtatccgcgattttattaatctggctcactacatgaaacagtttcaatccaccgataccacagccgatggcgggtaaaccaacatagactgtttcgtcagtttttgcctcaatgatatcgtgaacttgttcaaacataatctgaactgaactgataatggaaggcatgcgtgcgttaggaccagggtagaactgagtatacaaattgaatccccagacaccagtgtcatgatcaaacgcataggacatgttccccaaacgttgttccggaccacgttcatctaattggtcggcgcggtaaatttctgggaaatccaatcgaacatggttggcgatacctgcgcccatcacagaccaacaatttgcgccatgagcgaaagcattgatgagattaaaactcgtagcggctttcagtaaatcgccgtttgtgataacatatgtggtcatgcgtaacgtctccattgtataatttcgttgttgacacagctaacctggatgatacacatattctggcgatggtcagtgagtgtcataataaaactgtctgtctgtgccataacacaattgacagatattcctcgcatcatcaaacgaacacagatttcttgatataatcgtttcatgtatcctgaaactgaagcggactcatacacgcgttcaaattcatataactcgttcatttattcaggccatcaatagcgtgttcagaaatatattcaacaaattggttatcagctgccatctttaaaagttcaaataattccgttccaacggtactcccaaacatcccttccaatacaccctgaacgtcttcatctatttcttcgatgacgagtccatacgatttcaggcggcgtgaaatagcacggaactgggcaatatcaccaatttcatttgccatgttatttgtctcattatggggttaggcggcaattataccgccaatttattcaatagaggtcttcggaaaagagttctttgaaaggagtgttcttcaaacggacctgattagtttgtccggcttcacgggcgaccacagcggcctcttcacgggacatccattgcccatagttatcaataaacccttgttcacgagtgtgggtggtttcgataatacctgcttccttcaatcgtttgagttgcgtgttcatcaatttgtcgtgatgtcgggcgcttacaacgatcacatcaccatatttgttggcagcagccacaacacgccgatcaaacttggttgggtcaggtatatcacgagcatacaaggcgatgtgagtgtcgtccacgtcccctttcacttcagagtataattgacacccattaggagatccagtaggcaaagaacgaaaatatccaagtaatttcataatctaatctatccatatttcaggttcaggaatatcgaccatctccacggtttgattctttaattcgtggaaacaatctccgaggaattgtaatttaccatctgtcaaaaataaatggcaacggttatttggatcagaatgattcaccaacagactaggagtgaaagtcggtgattcgagatcgtgattccattgccatattgggccaggcatttgccctgaaatgcgataaggatgaagcatattacatccaggacaatgaaaatatatggaaccacccattgataataattttggagtcaataattttgacatatcacgcctccacttctatatcaggattgtatgggcgtaattctccaattaacttagggaataatacgaatccaaataaattaaatgaacgcatggtgccaaccatacccacttcatccgattcggttacatcagaaagagcgcaaagaggtatccataaatcttgctgaccttcatgattgacaaaagcaacttcctctttaataaaacgagggcataagatatcgaacaaccaatagaagaaagccagcaatgagagactgcggaaatatacaaacatggtgaacccccgcttgtttggagggaatttcaccctccagatttataggcccaatgccttttcaagatcatctaaatcttcagtataaagatcgatgtcttgcttattatttaattcttctcgttgaacttctaaattctttatctggttcaacaaacgttcacgggcatctagtgtaattgatgataaagacatcttgaataaatcatcgagatcgccatcaaaatcaggatagtctgtctggaataacgctttcagttccgtgaggctccggcgcatatctgtaatgacagcccacccaatgaatatggcacgattgttcaaacggtgtattttgtcggtcaattccttgataagatactgacgtctatcttcatagcgactcaggcgataattgaaaaatggaagcaacatgtctttccaaccatcgtaacggtgaataacaccatcttcatcccaagctgtccacacgggcttattagctgagtagagacggaacatctgttcaacctgttcgtcattcaacttagacaattcaccccgcttgaatacaacagtgatatcccaaccatcttcggtcgtatcgttggcgtactcagtgaggactcccgccttgtacaggggcaacagcactttggtctcataggttttagagaaccaaccaataggaacctcggtgatgttcaaagtggttgcattaacgcgggtgaacaatccacgactgtatgctcttccttcctcagtatagcctgtctctcctttgaaaccattccagtacggttttaagtccttcgggtcttcgccacggagaagtgctctcagggcactgagcacggacttaacgcagtgacatggggtgtcggtggcataacccgagccgataccattgataccattcacgagaaacatgggcaggatgggtaaaaagaatttcggctccagtttctcttccccaagataattgtattccaatatcccatcgtcctcctttcggaagattttacggataacttcagacactgccactgaaatataacgagcggaagacgcttcgcgccccatgattgaaccaaactgtccgtcgcggtcaaagtatgggatgttattcgtacctgggaacccctgagccatgttcacgatcgtaccgctcatgttctcaccaccggatttgtaattcgtgcgagcggcggcgaacatgccaaggcggtcaacaatctcctccttgttgtattcaagagcagcgaagagtatcttgcgctggcttggcttcaggctgtcaatcagctgagggatttgacggatgctgttgaccacggaaaactccttgtggtcagtgttgacaaagtccgttacagtaatgctcttggcaaccatcttattcatcctcggtttcaaataggcaaacatcactaaaccaggttttgcggtaatcggcggcctcatcaccgaagccattcttcagtgactgcttgtatccatcgtccagcgtgaccgtcgtagtatacgcatccaggttgtttagaatacgcttgaagtcttcagtgctgttaccgccaagacctttcagatatttcttcttcgtgatcttcttggcgtcaggtgtcttcaggaactcctcatattcggcattgttcatgaattcgtgcattatattaccacaccacacgcgcatgtatggagtacgaaggagcttcagcctaccctgacgaacgtattcaggccagaacgtacaatacagagttattaacaacccacggatatgaatgccgtcgtcatccgcgtctgttgccacgacggtctgtggatagcgactgatatcaagcggctggcctggcacagcaccgccgtgaatggtgcataaattcttgaattcttcgttcgccatcacctttgagcgcggggcgttcaggcagttgatgaacttaccacgaagcgggaacaaaccaattttcttggtatcacgagcgttcaggatagggttggatgcgctatcaccttctgtcagtagcagcgaacacccactgcggtcgcctctggcggtcgccggataatacttctcaatctcacgataatcccgtttgctgatatccttcttcgccttttcaaattcggcatcttcttgttcattgcgtaatgaagccagttctttactcagccctgctgtcacgaactcaagcgccttgcgaatcagtttatcgctgggcttgtaggacgtaccaaactggctcacaggagtcgtcatgcgctccttggtctggctgtcaaagcgagggttgttgatgtcggcggcgatgaacaatgacatgtggttcttgatcatcgctggcttcagttcggtcttgaacttcttaaccagctgagggcgtatcgccgccacgatctggtcagcaacataatcaacgtgaggtccaccgatgtgcgtggcgattgagttcacgtatgatgcatgcatgaacgcaccagatgagggagtgatagcaacagaccaatcatccgtttcatcaacagccgagccagcgtagaagtaatcaacgaaatgaccaaatcgatcaatgcggatttgcttgccgttgagaacaaccttcaggcgcgggttgcatgccgccacttcgaatgcacgacggtaaatcatgagcaggttgttctggtcaagccccttaacacccaggcgcgcataatcagggatccaggcaatagtggtgccgaactctttcggtgtattgccgatgaccggattggacttcttgctcatgttgtcttcaaacagttgagtataagacttcttgccgtcactggtagcaacgcggaaccactttgagaacacgttgacgagcgaagccccttcaccgttctggccgccggacttctggttgttgtactcctcgtcctcatcgttgaagttgctgcccgcatagagcgagccaaacaacatctcagggagccactcattggtgacgccatgcttgaccacagggatacccccgttgtcagaaacgatgatttgaccgttcataggggagaccgtgacggtgatttccgtcaagcctttaccctcaggggtcttgctgtggtcaacacagttggtgatgatttcatcaaactgtttgatcagcgcaggggagtactcaaagttgtcacggaagatgactttgtctttggttgggtcatacacccacaccgtccccacagacgaacggatactgcccagatgacgctctgggcgaagtaggatatgctctatgtgagtgagctttttgtatttgcgttcaatatcaattttgttggacatcggcctatcccgtcaatggtaaacacacaagattatgtatggggttggcttatagaccaaatttagtgcgccatttttccactttgtgttctatggcatgttccactttaacgatcgtgttgatatggttttgattatatccacacgccaatttggcagtattgcgtatcagatttgaaaccaggtccaccatattatcttctgaacgttgaggttggttcagtaaacgagtgatgcggcctttgtaatgattaaccatcgcaagatatttgtctatagtttcaacaccactgcagaaccaataatcctccaacgtatgttggctgagatcagttgtgtgattggccgtagccaaagcaaacaccatggagtcaaagatttcttctttggtcatagacccatgttgttgaacttgttgataatccacaacatataatagatccagagccgagatgataacgtccgccacttcaccgttcaggggttcggatgcagcgattccataaagcacatctgatagttcacaagactcttccacgagtttgcaatatacaaacacaggagaacgattaggatcgcgctggctgagttcatatgatgtgttgaagatagtttttagaaaattcatttcaaatatccttctagacggaagcggttaatatgctgtatgataaaatccatagagttttgaaaactgtatttcccctgatgttcataagcgtgaatcaggggaagacggctgtgagccaattggaatctcaacttggatggcattgtcagcaatttttgactcgtcgctgcgataatgtctcgtttaaatttgggatcatcaggggacaatacaacgatgcgtgacttggccagggtttgtccaaattgcttggcgtcgttcccataaagactacgcaagtcgtcgtggcgatattttagccaataccagtagccttctattgagttgaaattaccatcaacagtatccatgggatggtatgcaaaatgggaaaggaatctgcctagttcggtgcggcttcgactgtatatgttgtaatgtgtcttgccttcactatccggcgtcatacgttcttttggaacattgagtaatgggagcatgatatagggtctcaaatcaaaattgctgtgtgtaatcaggtcaaatctttttatatgctttcagggtacagataatagagacgccgccaaatagaactaacaatatgaatcgttccatcctgaaatcaggaagggaaagcagtcccacaaggctgtctaaagcaatcatgaacgttactccagtcagcatgcccaacttaaaggagttggaacctcttttgcaacgtagcataaatcacctccgattcaaatggggagttctctccccacacagatatctagcccagaaacactactaaagtcaaatttgtgagactggtccgagcatcctgcaacatttcacgggcagcggctaaacgttttacatcatgtagagtgtcagagaacacgattatgttagccttatcgatcctggtccccatgaaatgggtgattgtcgtcgtgctctctttcttgtcgacatcagggataagtaattcttcgcccgtctgagggttgcgggaaggatagaagaatgcatcacgttcacgagaagtatggaagacgccaaacacatatgcgtcgttttcgacctgaaactttttagaaagtagtttcttcagagcggctgtatgtccggtctggcggagcattctgtagtagagcatgttgccggagcaaatagggtctatcactcccctaatttgacgttccttttgtatctcatacgcatctgccacaagccgctggatcatatcgacatgggcattcatggatatttctattttacgtttgatcatagattgcatttggtatatcctcagttaggttccgctcatggggaacgggtccattatacacccaatgctggaaatgaaaaagaggccgaagcctctttaatcgtcgtattggcctgtcctgaaattccattttcgcgccttggataaatccaaatctttgaaaatgctgccaaagatctttttaaaatcagactcttcttcaagatcttttggttttttgacaggctccttaaccataccgccttcgggtttccccacatcggcggtcgtggttgccggagcagaagagtcttcaaggaaatcctggaagctcttcataaattacaccacagccatttcacaacgagtagacgtgacagaatgtccatctgcatcagaaacaacacaccaatattcgccttcatttgcggcttcaactgctgtgataaccaggttagcagtggcggcggtggcattagtgccagcatcgataactgaccccgcacccccaggagtatcagagaaatgccacacataagtgtatggagttgtaccgcccgcagcaacaacactaaaggttccgtcttgtccagaagtcacgtctttggtggcaggtaaatcagtcgacagcgatagagcgcctgcaactgtaataaccgtagaatctgtgtgattcccttcaaccgtgacaccataaaccgttacagttcctgaagccagcaatttgaatttaccaccaccgacatgtacagctttcgtaatatcaccagaataccaattaacgccttgatatgtggcattggcaggggataccgtcgctagcaattcaacaaattgtccgatctctccgctggctgtagctggagtaatcacaagtccggtggttaaaatagcattcggatcgtggatttcataagatacttgaaagccgataggcttggtgggatccactttggtgacaccgtccgcttgtaaattgctcttggtgagcagagcgtcaagacctttacattcggtcaggatctcatcaggatcttggttgaggctggtcttttgccaaccctttgcaacacccgtcacgacatgattgcgttcggtttcgggtttgttgcggacaacttccggtgcgactaatactgtgattgttggcatggtttcctccactcatccaaatcgttgttcagagccatcatcagctcattatctggttcgtatgaaccggaacataaatcatctcgccaaactcctaacgtcaacagcgctgctttaagacgaggatgttggttcttatccgttttcaacaccaacattcggcacacggcttcggtttcaaaggtattgaaaagaataatcaattgattgataagtactcgaggacttttccctggcacaaatcttttcaatgattgattaattagcgaaattcgctgaatgtctaccatcaaatcatctcttgagacaccaggggtgatataatgtcttagagcgtactccatcaaattgctttcgtcaacgatagccatattgaattataatcagcagttatacactgggattatttaatctgcagtttttcagtcaattgcgagattatttcattacggtcagtcgcgtcgatacccaggggttccgcaagcgttttaagatcattgatatcgagcaaaagaagaacttcaactgtaatatcggttgataccacagtacctggtttcatcggcttaccgaatttatctaacccataacggcgcaaagttccggcaagcccggtgagttcctctagaagctccttagagccgtctttatattcacacacccaaccgtagtcaccagcgtagacaataccgccgtcgccgccctgaggcacgttatccaccacgcgttgcagataactgggaagtttattttcttcctgtgcagtgatttcttggatggtttgggtgttttcaattttcttttgtgtcttcgccatgatcagttctcctatagactgtaatatatttaccacaacaattgcagcaataaacatgcatggaatatccgtcgtcgttaaagtcacagccgccacaacgacaaaataatttcacgaactggtcgcctatgacttctctcggaggaaaattcctgaacgagtattccttgagttctccatcaaattggaatgaatcaacaaggaactttgtttttcggcgcagtgatccaaatgcctgttgctcatacacgaagtaaacatctctggtttccatatcgacctcaaagaaaaggaggccgaagccccctcttttatgtcagttcgcggttacttgtatagaccttcaacatcaatggatttatgttggcggttgtattccgtggtgtaatcgtctaataccagatattcacagcagcggattttactggagccataatcatcagtgatagacacgatatctttcgggttcagtttgcaacggacaacgcgttgtcccaggctaccaaaacattgtttcagataagccagagaacaaacgtgaagaccgtaagagcacagatctttgttgttatcgttcacaaatgagcgagccattcgaacaatggtgccaggagcattactaacctttcctgtgcgcttgtccatgtagttaccgcgaacggatttatacaggatgatatcaccgtcttcatgaatttcgatatccgaatatgccatgaattcatagattcgaccggactgaaccagcgcggcgctcgggttttggaacattttgtccaggaaacgacccaagcgttccaagttcggataatcgccttttaatgccaaagccaaaatgcgtttggcaacggaagttccggtgatatcatagccaccccagcgaacacgattgtctgaaaggtcaaccatgcctgtggtgaattgtttgatggcttcacgcggtttcatcagagtgtacgctgttttgacgtcacgatttttaatcgcttcaacaatacggtcatacgcctgatggcttgtggttatcgtttccggtttgccatccataacgataacgatgctttccggtaaaatcatgatctggtgctcattcaagaacactgacagatctttttcgttaaatgagttttgagtttgtttgtcgccagactcttcctcaacagccgttgtcttaaccgagaaaacaacagacgacaacgccagtcgaagtgtacgccccagaattgcgtcaaagtatcccacacgccgacgggcatcaagggaaccgcgcatcgggtatgtttcgccgtcatgttgtacactgattaccacgccgtcattgactttggtaacgccatcaaccagtgctgtcgccaattcggccaacggagaaatgttttcttcattgaagcggcttgaccgagtttctaacggtttcagttcttcgcttctgaaaagtttggcctgaatagcagcagaactatcgtaacgcatacagataaacactgtttggttttccaggaaacggccaacataccattcttcgccttcagaatcgacagacaccgaaccttccagaacttcataggtgtcggggattttcccttcttcagtcaattctttaattggcgaaatcacgaccacgttgttagattctttgactatacgaaccatcagggcgcgggcaattgttttggcactgacgccataaactgatgccagagaagaattgctttcacctttgctgctcttggtttggatttcttggatctgttcagtggtcagggatttaatcgaggctggatcaacacccaactctttatccgacacgcgttgtatcattgagcgcggtacgcccaattcactggctattgaactttgacttctgttctcattcaacagcttgagaatacgtttttctatagcattcatcttgtagttctcactgtggggtaatggggctattatgccccgagaatacgttttgaaatagccttcacaaatctagtggcatcttcaatcgcgttgtgtgccgaaggctgatattttgacatcatgaacttcaccaaacgatgacattcaactcggtatcggtttttggccttttgcttttgagatatagtgctagtgacctttatctcatcaaatccttcagcagttaatttttcattcaataaatcaacaacttgagttaactggttacaggcgcggaaaaatctcgtagttgggaaaggcatttctatacgataatgtaattcctcaacagccttgcggaaataaccgtaccccgatccagacgggtttcgtttggccagcatacgaacaatcttccattcttcttcagtgatttgttgtttcgcataaccgatgattgcttttgccctgtgttgatgatgggcatagccatccgtaacttttccatgggaatcaatcaacagcttggcaccataatcaatgcgatgagacatgatgtttatgcccttgcgaagtttcaggagtttcttagattcaagcgcctgaaattcatctttataatgctcaagcaacatatcaaacactgcgtcaatggggatgaatacttcaggaaatttcttcatcatcgggactgatttacggcgagcaagaacgattgttggatgtgctcccagcaaatcaaacaggttttcgtttccattctcgccgaacttcgttctttccatcgcttcacgcaaatgtccagccgtggattgtcgtaaggtattaccatgaattttatggataccttctcggccagatacttcgatatatacgatctttttgcccgaatcaataatatctgaaacggtttttccatctaattcataaacgctgtcgaagttaaatgaattgtaatggaaactaaacatcccaggatctttttcaacggtcgggcgaacatattccatttcatgcatgaaatagacttcatccggattgaataaatcgccaaagacctcgatcagtttatcggcaaagaccaggtagtccggcgttgtttcttgtacaccgatccatgattcaaaataagacttcaattcctcatggttggtaaatgattctttatgtgccgccagctgattccgattactgaaataattttcattcggtttgacgacgataacgacattgaagttattgcgtgctgtttgtatcttcaggtttcgagcacgattcgagttatcaataataacagcaatttttgatttagatttcaaccaatttccgacagagttcacataaatgctgtcgtgtactacaccgttgtcgtgtacttcaatttcgtaaggactaccaatagaccggaaaggtgcatccaaagagtaatcgcgcacgacgtccggcgctccgatataacggaacgtgcctttgtagaaacggttatctgcaggaaattctcgctccactaatttacggaaaccgctaccgaacatatcagtcagctgagtctttttcatatacaacataagaggacccattgactccgcttggcgaagttcatacatcttcgcttgcatgatattttcggccacatgcttgaacacgcggttcaggctttcacgagtgaattcgtcgtatgacaagtcttcgcgtgacggcggtacattcagttctccaagttcgaagaaggtataggaactcgggaatttttccagcacggtgcatatttcagagtccaattggtcgagatctatcgggtaagaaaccccgcccatgacagcatagtggatgttgtcggaatctgacttcggttggatatatgtgttgcctacacggttttcgaagttgatgtcagaccaacggaaagaataactggcgttttcaaccttcggtcgcattgaaggtcgcatcactcgacccaaacggaccaactcttgttcaaaggcggtaaaacgctgattctttacaggaactttaacagtcaggccgttgaactcatcagtgtcttccggtttaggatcacgggtatctaaatctttggtaatgagatctacagttgggattctgtccgcattcagatacatcaggaagcgatacagtttcccttcatagcgactttccactgtaaatgaatcagataccgcaaatggggatttagaacctagcccaaacgcccctatttgcccgtcgtcgtcctcttttgtactatggaagagtgttgtatacaaaccaggtccacgaatgatctcgccatcctcagcgcggaataccagtaccccattataataaccaggtgtaccaataacagcagtgctatcagggatttcgtcttccttcacgacgatattaccttcaaccagcacttcatcttcacgcgctgtaataggctcgccgatgatcatttctaatggaagaccaaccccataatctttaatttccagccagggttccataacatccggtaaatgtacaacaacaggagttcccttgggggcaagatgtttgctgtatcgttgaggcataggagtgagaggagcataatgcgatggcatccagcgttgttggcgatcacgcatattatgcgagtctataccattacacaacgtctcacgcaaagacgccgcctctttatatttgtagagacttgaaaataatgtttcaaataatttatccgtcatcttgacattgaatgtctgcccgacgatggtagaagaagagcgcacgaaatgctcggacttgcgcattttcatagtatagaattcctgttcagatcaaagaagaacacattattgggtaaggttatgatactggcgggtggttcaatagaaaagcctccaatcggaggctttaatcaggattaacttccctgaacgtttacaacggtggcgttgaaattgaatatcgggtgggtggcacctggctctaacgtctcttccagataacccacaatattgcgtgtcacagatttcggctctgcgcgggaaggcttcgttgcgccgaggtcaataatgctcgaagtgacttgttcaacctgagtgtgattgacaacaaaatgataacgactaccgaaggccgcaacttcaacttctgtgatttctaaaccactcaattcagagctgttcaactgcttcacattcaattgagagcggaggtgctctacgatattactgtaaacatattgtggtgttatcagctgttcaatagaactcatgacatatcctcatttctttgggggtttattgttacttaaacggatcacctctgctcgaaggaagaggatttcatcggccatcattttaccttcttgaaacataacatgacatcgtgatgatctgggttcaccttccgataatgcgtgaaggcgatattcggggatgacgtcatttacggcgtttctttttgttctttccatgggtcttggcctttttaggacgactgacggaaggacgagcatgcccaggttccccaggggtacacccttgtgcataacgatgatcatcccagagacgaccattatgcgtcatatcttcagccatggcggacaaagcaagggttgctacagccgcagcagataatggtttagacatttgtttcaccttttgctgcatacgacagagcttttacgatcataacagccaaaccgataggatcgtatgcattgtcgaattcttgttctatgttctgacgaatttcatcagaagattgtacttcagagcagttcttactctgtctgcgttgctcttgctcgactagatctttgaacagaaggtctttgaaagaatactcatcgaagttttcaagataccgttggatgcttggttgttttacatccatcgtttgacgcaaattgctaatagttgaacttgcctgttcgcggtgtttgttcataacagcaaagttaacgggcttgccccaagcattcatcactggattagagcatacttggccaaagagatcttggaaggcatcatccatgtggatcaacgcagtggccaattcttcataagttggtttttgagtagtcattttcgtttgctccgatttttatatttcttagcctgacgtttatgttgggcggctccaataattgcaatctttcttggcttgtcagacataatacattccataaaaaagaggatagagcaataataccctatccccctttattgaagaacgattagtctttggattctactttatcaccgcgcagatcctggacgggtttgccatccggcgacatgatgtagatggtgttaattgtctcaaaccaagatagttcatcaactccttcagtgtatagagttaccggaactgaagttttgatatgataaccatcgttatttggggtgccatcagacttcagcactggctccaggaaggcgaaacgaccatttggataaatcaccccgcctttaaacatccggatcttcttgcgagggttgttggcctcgccaaggcacacgaccagatgatggatcatctccggacgggtaagcaacgtgtcgtcacggatgataccctgtttaatagcggagatcaccacttcgtccaacagcgcctgctgctggtccagcgtcagtgattcagtaagtttcagagccattattcgtaccccaaacatttgtttaagaaagtcaggacatcatccatggtcttgacctcaatttcaatgccgagttgttccgccaacttgttggtgttctcgacgatctctttgtctcgcgcagacatgatgtcagcctttgaagttgatcggtttctgaaccccagcgtcaaacgcggcctgggtacgggtatcgctcaccacctggcaggtcttgtcaagatcgatgcgcgggtagccaaacacaccagcaatcaacgaaccagggaactgagccagcttggtttcgtacatcgcgcagatgtcaagcttcttggtctgactgatacggaactcatcgcggcctgagctgatgacaacctggatctctttgtacaggctggaatcgaactgaatgttctgttcctggatccactgcatcacagccttggaaccatcagcgccataacgcccttcgaatgtacctttgatcacttccttcaaaccgtctttgtacatgtcaggaatctgcgccgtctcctgaactttgagcgtgtagttgctcaggtagttctcggagtctttgttgaacttcttgacctgctgctcagtgcggttgaagtcattgaaatagctaaccacactgccaatcccaattacagcaaatgccagaatcgccaacccaacgatccaaccaattgaaatacctttacgttgtgacatctttacttcctcttgttaaaacaaacgtttataagttcgaccattgtactgcatgcggcctaatatgaaaactgcggtaaaagggatacatccaaacagcacgacgataatcacttcccaagttttaagatctcgccatttcaaatattccatttccttcatctcaactcggttgtatcccttactgatattggccgcaacttcttggaacacggatatgcccattggtttcccagtcaatgaaagtccgttacgagaatggagttccatgttgttcatgccgtcggcaaatgacgtagacttaccccaattaattttcatatcctttgaaatatcggtgactacaataacatcgttctttttaccgccagaccatgcatatagaagtccctgaaaatattcaacaggctggccagaagtcactacccacacgatattgacctgacgtgaagcacctaatgttttcagagtattgttcagataatcattccagtaatcaacaggaatgtccatccctgacatattcagaacacgagttactcggtaataatcatacaccctcggatatgaagggatatgctctttgaacttctcagcgaattgctggtcagatttagagaataatgaatctttgttgcccagcacataattcatatatgaatgttcacgtgccgcaggttccccgattttaacttgtgcccaacgcggaggctctgtagtaccctgacgatcaatacggtcaatgctcagatcaccgacggttgttaatacgtcccaatcaacgtcataagagtgctcatagcatgtgctgcaatgacgcgtttgtgtacaagatcggttatttcctgaacctgtacaagatgttgtgtaataacacatacattcataagaatgttcacaacccaccttattccgttgcttatcagttacatatccattcaggatttccacgtccccagaactaccaagggagaaagcagcatataatcccgactggataaggcaagacaatacgataacaatggccgtggcgatcaaagtctcctttggctcatgagtcttgcggtgatatatcacaaacaagatggccaaaattacaggaaccacaaacagaaggtaaatcataacacaccatccacgagttgttccttcgtcataccaagaagttgatttttatgctcagatttagccccatttttcaacaacagatcccaacgttcttcatgatactgtggagtcattgcatcgtcaaaagaaacatggtggcgatgcataattgttgcaacggtatgaagaacgattgctgtttctctttttaaagcacgtgtatccgtgatgtggtcttcgtattcgattcccatcaaatcgagttcttttcggatatatgctatagacaaatcaccttcagaatgcaatttaacaatagtcgctttcttagggtggagcgcaccatccgtttctttcatgaacagatcgtacaaatattgtttgtattcggggcgcttcatctcgtaatccctccggcacagatgatattcagttcaccgcgacggaaaccgccttgcccaccgttcaatttagtcggaagcgtatcttgaactttttgtgcgaactcttccgacactaccagatgacccttttcttgtttcggggtagattcaaaatagtcgccaaatgtcgcttccatatcgagcacagacatatcttcaattcgttgcattgccatgatataggacctcagtataatttcgtataaacttgtttaacgccaatatcggcaggagctgtgctctgctgcatattatagaatgccaaatctgcttgttctttgttttcaaaatcaactacaactgtgtgtactgatattgcttcaccctgtccataagcataacaacggctggtgaccaatattttgtattgaggtgtcataaatcaatccttgcaaaaagaaaggggaacgggctaataataacccattcccctttattgaagaacgcaatcgcaatcagattactttttcttcagctcgtccagtttggcttcgatttgttccaggatgtctttacccttctgaacaatcacttcaccgtcttgcgcgtgtttgcgaaagaccagagcgccagcgatgaagccgacaaccacaccaggaagcgcataaaccaacagatcttgtaacatgataatatcctcactttatagcgaggatatttatatcagcgtttcaagagtgtcatgaggttcacacttttagaaggatacacagtaccatttcggctcatggtgtccaatttaccaactgacaaacaaacctcagacggttcccaacgtgtaccctgctgtaaatgccagatatgtagaggaacacccgtctcaaagaacacgggcatcttccacttctggacgcctggaacgtgccctgcaggagctttttcgcacaacagggtatctggtagccagagtgaacgggcttcccagagaggtgcacaggagtattcaggccaaatacgttccatataatcgatctcgtcccatgtacgctcggcgtcccacccgatgtaacgggtattcttcagtcggaagaatttcttcagccaacaagcaccggaggtctcaaagttcagacgattgataaacgtgatgtggccaaactcttcaacgcattcctgaaacgctttctcaaggtcggtttcgagtatatcacattcttcgtctgtaataggacaaccgttaatctttttcttcccgtgcttggtcacccaatcatcacggttggataagaacgccgcgccgttacgattggattcgctaccatcacggtcacgcaacatgaacccagggacgtctatatcccatatgttaccgaagaccatgttcagggcttccaggaagttccaggcgctcaggcgaccgaagtatttccatgacatcgccgtattccacagttcggtgaactggtcttccttggtagtgcattccaacatccaacgaaaagaatccatttgggttttaccacccaaccaatcgatataggactgaacgcacggaatcattttcgacttacgataacgacaatctgtatcgaagcgcatacgatcaaagttcaggttataccaatcagaaaatcgctgaaactctttcggagattgtgggggtacaggaaattcactgtaaattgtccatggtccaatcccattatagcaacacccccacaaaaaggccatccaaatcttgcgctcgatcttttcgagctgtgtgatgccttctgtaacctcgatggcataatccatcagacggacttgctggttatgctcttcagtgtaggatagcgcctccacccatgccttgagcaggtacatgcgattttctggcttacgataatctactgccagatcaatcgggtatttccatttttcaggggctttatatccctgcggcattttagacagatgcgtcatccttcacctcggcttcgagttggatatcttcggggcttactacgggctgtcccagagaccagataggcaggaaacgacgatcattcgtcaccaaccaaatttgcttggcggttttcgtcttttcggaaataaaatgctgggcattgacctgggtacgagccaaacccacctgacgccaagaaccgaaatccttagaagaaggattggtattcagttgaaattgtgggacgattttattctgttccggataataacggacagcgttaatcatcttcgcatcaccacggtaatcaatagcaacagcgacaacgttgcgtggaatgaggattttcattttaattccttttgaaactctgaattatgttatagacgaaaggcgttaataagatagcaccccccaaataatataacgcgtaaacgttcaatatttcggttgaatccaactcaacgatgcccaaatatttcatgacaccgacaagcacagctgtgatacacatcaccaatccgacgaacacaaaatatttagacatctgtttcatttctaacccttactcaacaggccgagaagggatacagaggactttgttacctctcccttggcgctcgccttggttttagatttggtcgccttctcagggactcgatgcgctagaggatcatccccaccgacttcacgcgtcattgggtcagtttcacagaatccgtagaaatctttgatgtcgagatcgatcgcaccgatatccatctggtatgcgatggcatcaccaacaacccaaagaggagcatcaaacggcatgaccgctatttcgctgcggtggtccatgtcatacacattttcattaggcttcatatatacgatatcttgaccgctgtaagcatcttctaatacattccagcccagcgccagcatttcagctttcgacttctcaaactccttggagtaagattcgttgcgtgaccagccggagtcccctgcttccttaccagagcgaccacggatgcgtgcatcatactgatagcggtctgggtatgcaaaataaagcaacgccagagattcaatcggatagttcttgaacatccattcaggacgccatttatcagatagcatgaggggttcaccctcgcacaccaaagtgtaaccctgagccagccagcctttgaccagatcacgggcgatatcacccgagcctgtggcggcgtggatggcgtccatggaagtccaggaagccagaccggatttgttggacacggtatactggccgacgaagattaacttcagctcttcgaacttcaggccgaatgggcgcgtcttgtcaccaatggtgtaggtgagttcagtaggctccagcttagttcggagccattcgatgaactggaccactctcgtacccttgcccgtggccgaggtgcctttcactacaattattttagccattatgcatcttcctcaatacgccagctcacaccgcccatccatggtgcccagaacacagcaacctgcgggcggccattctcttcaaacttcactttatactggtcttcgaagttatagaaaatttcatcgtcatctggtgccataccgttttctatcagaacacgctttaactccagcgccgcaccaacggaagtgggtgtggtttgtttacgaatgaagttgttcacatggataataacgccatccatatcgctatcaccggaacgcttgtctggggtttccccaaacttggattgttcttgcacgatgtcctcctcaatggacgtattgataatggccaatcaggcggttcaccacttcgacaatattgcggttcaggaagtcgaagtggttctggccttctactggtttaatataaagcatatcttccgagttcgaaagatccagtttttcataatttccctgaggaacaaagatgacaaaaatattgttcatcgcacagggttcaacgtatttcgggttatcatcaattaaaacatcacctgccaaaagatgcttttcgtcggtgctgacaaacccgttgaagatgccagggaacttgtcatagacaaactggcgcttgctgcgctcgtgttctggctcacacttagatactgcgatcaattcaacattttcaaagtcttcgagaaggatcttcttcagattcaccaggaactcgtaagcgcctggaagcgggttcatcttggcatacaggtccggcatgcgccaccaatccataggatcacgtcctgtaggtgaatccatccattgaccagcaacaaacacacggcgtgtcaaccatgctgggtgagcgcgttcccgcatgaggatcgccaaatcaccagcatgagccatatagcactctttggtgatacgctggaaatcattgggataatcatggcaacccatgttttccgcagcagcttttgaattggaaatattaaaccaatccacccaaggggagaggctatcaaccaaagtcaagtctacgtctaccaggatacgatacattccaccgattttagaattggcatcatacagaccaacatgattattcatcagacttctccacttgatcataaccagttgcggtgcattgctcttctgtgcaatctgcatacagaaccacagaagattccttcacgtaagtggtacagtgataggtttcatcttcctggatggaatcatcataattgcaaacagtttgcaacggaccaggagtggtgttatccaacgcagcatgtgccttgatggtgtcttccatcttattcaccgcttgctgattggtgaacgtcggcgcatcgaggtcaacagtagacgcaacgtccagaagggtagagcatgcagtggtagagaaagccagagcacacaccagaagaatcttattgagtttcataacaatacctgttgtcaaagggataacagggggaattatatccccctttggtttattgaatcaacctttcttgcccatcatggacaacagattaactcgacgtttatgagaccaaccaaccacatccaagatagactgaattggctctataaaggtcttttcaaacgtggtgtggtaatccacccatttatccaatcccaattctggaggaaggaagtcagggaaggctatgcgatcattccccacaggattgccaggtttcaggttgatgattttaaccttatcaccggattcgatagggggaagaccgagatcttcgtgcttatcgatcaacttgttgtacataatacaagccttggcagcaaagtgcgtaccgctgatgtaattcccgttcgcgtctaaccacttctcaatatcgctcacaccagatgcctgagcgatatcatccacggtgagttccatatattcctttttgtatccagcgattaattcctgaacctctgcctcagtaccgagcaggactttctcataacatttaaccagacgctcacgacaccactcaggagtggttgatttacgcgcttccaaacctttgaatttgatctttggcttctcatacttgatgccttcgctatcgtatactgccatgcaatacatcttcttggctcgccatacagcagacgatgcgatgacctcacgttcccagaccatgcgctgctcaaacccgttcatggtattacacaacaactgcgcccattcactggtttttggctggtaattctctttgatccattggtcaatgttatcaacgagtttgtgatggtccttttcttcaggccacaattgcttgaccaggcgttcaatgcaaatgtagttggagtctgtatcacctgcgataacaaaatcctgaccagtggtgccacaaagtttgttcagataatcatcagtgtggcgtttgttccatttattgatcagctggccggaagtggtaattgcttcagcgatgttgatgttgaagtattctttaaaccagacgttagaaattgcaccatatcctgcgttcatcaagatcttcagaccttgttgtaacgtatcttgggcaactcccaaatcttcccacttgtgcatgacttcagtcaggtgatcaaggtcgatatgcttgtgttcttcataccattcaggatcgtagaaacgagacttcatggcagattcaaggtggaaatcacctttagacatttccttacaccaaccagcccattgctcatacttcaggccagttgctttctctcctttacggtcagcgtatatgcctcgcataatttcggaaaggaaagacatcttctcattactgaagaattgaacgttcggggtaaacgaaacgttatatcgacgtaatgtttcaaaatggaattcacccagcgcgaccaattcatcaacaacttgtatgcgttcatcaatagcacgctgcagcttgtcgtgaagatttttgagatgacggcgcttgttcataggcgttgtcatatcattcatcgctttggtcaattcttcacacatggactcaatgatatcgcgacgtgtgtgcttgtcagaaacgatagtctcaggaccaaggttgtattgctgtatgatgtggggatacagggagtttaagtcctcagagaatacccagaaatatatccctggtgcaacctccatgacatatgcgccttcaaagtccgttggaccatcatatacacgctgtattttagggacaatccccttttcataaagacgatagtaacacatcgccagccatggtgctacagtcccaagaccgtcttcatagttagatttggtgcgatacgccaatacgaacatcagctgtatcaaacgcagcttttgttctaaacgccacacgagtttgacgtctttgataccatatcgggtgtgtttgcaataatcattaaaatatagatcatacaatgacttgctttcactataatccaacttcttctcaccgagttcacaataagcgatccaatccagtgagtatttttcgcgggttgtgtacgtgtgtttcttgtaaacttgcatgtagtccatcataggacaacccacgaattgataagaagtcacgtcgcctttacggtctttgatgaaacgtttcttgagtttgccccaaggactgaggcgctctgcctgggtttcgccgagaacctgcgtaatacgttcaaccaagtacgggctatcaaacgtttcgatgttccagccagtccaaccatcaaattgacgttcagaccaataatctaagaaggcgcgaagaagttcttgttcagtcgtgtattctttgtattcaacctcaagaccacctatctcttcatcatttggatcatatttgaatttatggcgatccttagaacacggcataccccagacatagaacttgttggtgttcatgtcttgaagctggatgagcgtaatagggaaggcggcattcatgttctgtgtgatcttcccattactatcaatgatagggaactggtcagtcacgttgttggaaataaaggaacctgggaaatgctctcgaacgaaatcatggttggccaacacttgcttatggaatcggcgaacacgcgcctcgctccccttaaacgtgtgggattcaatcgtcgcgtgaggaaatgggcctttagtcatttctccatcgcgccacccagccgagaaaacttcgatatccacgttggcgatgtgaatatttgagtaatcgggggtaatcatcccaggaaaactatgcgctatgaattgatatgcataatccgtttgtccgtaaacggcagcgccttcgacctccttatattcctccagatagttgtcggcgtcacgcattgacgcaaattttttagacaccaacggttcattgaggaggccaatcttttcaactttggaataatcggctgtgggtaaatacaaggtgggttcgaatttcttacgcaacatacggcgattgccgttgtcgtctgcaatacgaatcagaaggtcgttaccctgtcgggcaacgttcgtatagaaaacagtcattgtttgtctccggttaaccgtgccgttagcacgtccccgtaatttagccgaggaacgttcaatgaccaaggagtttattatggcaatcccctttgtgagtatgatcccaggggcgctcaagcaactctggaacctcgggactgatttgatccaatacaagcgggaagttgttcaggccaaacacgatgtcaagctagaggctatcaagtcctcctcagattgggagctgtctaagatcaccgaagtcggcgggtcttggaaagacgaattctggactatcgtgttagctgttcccgctatcctcgtcatgactgctccggttgtggaactaatcctgttccctgatgagtatcataaaggggatttcatcaaagcagtaatcggcggcctccaggcgctcgataccgcacctgaatggtatacctattcgttattgacagcaattagtgcatcattcggtatcaaggggtataaccattacaaaagcaacagccggaaggcgcaagctgtggatgctctcaaacagtttggggtcaaagtggttaacaaagatcccactgttgttactcagtcgggttctcccgatcttgagtctgctcccccagctgggtctactgcgacctctggtgcgtggcctgatctgaagaaataagttagaggggaggaaactcccctttcttttggtgttaaggaatgaaagtggtaaacaagagattgctcgtcacccaaggcggggcgacgatagtaagaggaaggatttacgaacagattcagctgggtgatcataagacacgcctgactgtcaaagcaccggacggcggatggcccacaaaggtggttgtccccgacggcctcatagaacgagggaaagcagtgtacaaaggagcagaattcaaaatttattaaaatatttattgaataaagtttacttcttcaataactggcgtatacttctaattgtagtacgaacaaacaacaatctgaagagaaggaaatacatcatggcaatcgaaattatcgctcaatccaaatccggcaaaactgtattgttcggcaccgaagaaggccgtgtaatcaccactttgggcacagttgacggcagcaaccccgtaaaaggtggacgcttcctggttgagtacggttctcgtgtcgtgatggtaagtaaaattgatgggcgtgatatcaccgctcctcgtcaagaattctttgattcacgttctgccgccatgcaattctttttcttccagaaacgtcacgaactgttcgttgaagcgaaagacgcaggtctggacgacgcaacggctgagctgattgccgaaggtaaaatgactcttgagactgctctgggagacatggattacgaagccgaagcgcattatattgaatattccgcatatcagaacgaatgtgaagatgatccggaagatgatggatttgatccagatcttcacggcggcgaggatgacaatactccttctctggaagtgatcatggctggtggtcaggccgcttgggaatggcagaatgaaaagaacgcatggttggattcccgcctctaatatgacaaagccccttcgggggcttactttgaactgaaggactatattatgatttttatcgtgtgggtagtattcgcatttctggccatggccgacatcggcaagtatcatctaattcccagtgatctgactgtcctgctgtacggtatggtttcgtacatcctgggttatgtccgcgcccgtcttctgtctgaatgaaggactatatcatgagcattccaaaaactcctgtccgtattgccgtcgagcctctgctggttgatgctgagaatgctgctgagcagtacgcgaacaaagtcatcaagaacataatggacgatctggcatccaacggctgggacgtcgagaagtgcgctccgtggccatcttcggttggtgtcaagtttggtgatccagattaccaacgaatgaaatcaaaacatgcgctctaccgttctttgacccaggcagtgaatccttcttacatacctgggaagccggatattgtcaaagctgatcctgagcgccatgctctgtttgtcaaagaagcacgtcgaaacgctgcgatgcaatatgagtctttcatttgtaaactggaagacaaaattggtgggcacagcgcggctgtgctgaatggttcccatatatggggttattcaatcctaaccgtgactacaccagaggggatcgagcgctggaagactcagacgattatcaatgtgtccaagctgggcaaactctttaaccaatatccaacgaggaaggtgaaatgatgaaaaacttcgcacaagaattacagactacgattgaccaggctcagatttgggaagccatggggatagttgaaaaagccgttgatatctttgtttgcaaaatgaagaagcaactcgtttcccaagcaaatcgcggagttgaaattgatgacatggtgtacgctgttggtggccacaggtttgacgaaatattaaccgcatttcaacaggcatataagtctcaaaaatacccaacagacaagcaagtcgaaaagttcaaaacattgttggtggcgagtcttctgaaggaaggtttgaagttcgaattacacaagaatgaaagaacagatgtcgtaacaatgagattcacagtacacacaaacgatatctaaaacaaaggggcattaagcccctttgtttatttcccatcccaaaagtcgtcttcatcgtctacttcatcaaacgcagaatccaattcttcgtcagagtactctggaagacgaccattctgtttcagatattcaatttcgtcatcttccaattgaagatccgtggctataaacagcgcccctgtttcttcatcgtgctcaatactgtaagatgtatattcagcaccctggactgtaatgatgatagacgtcgggtcttctgggtccacatgggcttcaaacgggataccgtcaatcggggcatcctctaaaaatgtagcaagagctacggcggcgcggacgacgtctgtcccttgcacggaagcataaatctcttcagcgcctgtttccccgcgctcaaagaattcaatcgtagtcccgtcttgaacaccagtccagataggcatgtctgctggggaatccgcttcaaataatttcgaaattggcatggtaatgtcctcttgaataaaccgctttggactatttaggcttctctggtcgccaaccctggcttaaacaccaccatttcctcaacgttattggcactcatagaggctatggaatcgaaccctttgttgataagttctttcacattatcatgtatcttaccaggctttatccccaactcacgcattttattttcccacaccgaaacattgttggaatctattacaagagtcttcttggccttgacacgaaatgccatgacaataggttcaccagattgacgatagcgcacggcattttctgcgtagatctgtgccttttctggatcccccgtcaggtaaaacccagcacccatcgtaccagagtcagtctggccaaatttttcaaaatcaaagatatcgaattcaacattagacccatgatacaaaacgatattcgtaaaatcaggtaacgttgattcgcgataaaattctagaaaggttttcatagataccccacactggtcgtatggggtatttaggatggattagctggggacaaactcattcacagggacatgttcaggtattgataattcaggacaagcagctttcatcgcttccagatatggcttgctacccacgatccaaaacaatgagttctcattaagcaattctggtttgtgttcctgtatccatgtcatcaccttcccttcataacgagggtgcaactcaatatctccccactggtaatccatgaggtcgttgtaacgaacccaattagtggtatgaagatcccaatggtgtacctcaaaacgaggaagcacgatatccggctcgttctttggcctgataccacttaggaggccagccagcgaaacgctcttagaggcatgctggagatccttcttaccatacacataatctgggttgtcgtgatagcgacgagtgaaatcagttatgtgagggaggtttgcttgtttaccgataatgcgcaaacgactttcaatgaaatctaatcggtttgggccaatcccgatcagatagacattcttcagatttggtttcggatgcatcgccaaacctgtcaggatactggtgcatgaattacaagaaccagcaggaatgatcagatcagtgatatggtctgggatattggcaacctgctcaccacccagcatatggaatccagcaatgcgttctggtgaatgaacggtatgatccaatgtaatgccatattccagataatacgcctttggatttaattgttcaatgagtttcttacagcgcggctgaatggtgctattgtaaccagatccaacaaagttgaactcactaccaaaccatgctgacattgaaaccatatcatgattcatgcatgtggttggtttagtggcacccagcacagtggttgtcttgccgccgaaatgccgtgagactgccgtcgccataggggactgcggactaccaacgacagtaccatggataagatctggggatcctccagctttcagatgctcaaccattagccagatggcctgacggagtttgctgccattgatcccctgcttgccattcatatagcatgacaaaggcgcgaagtaatcttcacgtttgaaccaaacctgctgattggtttctggattggcaatgagttcgcacggggtgtgtttgtagagataatcttcccaatgaaccaaatcgcggttgaaggaaagggtatcgaagatggttttgttcattctatttctcctgtaggcattggcttatcatagcgtatctacaggagtatagaaaaggagaatctcagattacttagtcaggaagtccagcagacggcgaaagaacgatttgcgttctactggtttcttcgattccgtcacaggatattgaaggataggtgggagttcagggtatccatcagggaaacgtttatccagatcatttaaactacgactggccagaggagttggcttcaaagatttaattgcctgattcaaatcggaattatccacgacacccacttcttcaccgaccgtaaagcatggtttgattagaacggatttcttagactgctcatactgatgagcgatatccaggtatttcttgcgctctgcgtagaaatcaacattctttactttacggcggcgagcagtagaactctcttcaccatagaaatcataaccatttttgaataaccaattcacggcttgctcaaatgtattgaacaccatatacaaaccgcgtcgaccatttaccatgtcgtcttcgctggaataaccaatgaataccgcttgccctttcttgttgatatcaaaaggagtacacacaccagtttcttcaagtgatcgcggagcaagatgcccgtcccgatagatatatgccggatacaagaactcattcagagtacgtttgttccctttgtatgtgtacaggcgagcaaatggaggcaggtcacaaacgacatcgttgtaatcccaaatcgtaacattacgcatgttgtttcctcaaatcaatgaaggggtttataaacggttaaattttaaacccctcttcctcattgaacaaatcaaatactagacatatagagtgggacaacctgctcagcgatcaacacgacctcggtaatggtaaaatcaccaccataagagtacagtttcatttgcgctccgttggtcgccaggttcccatccttgtcaacgctgaagaatgtaatgaaagacaaagtgtccggcggtggtacgctagcatcacgacttcttgataactggttccccacagaacccacaaaatctaatatcatagttctattagaagtagacccagaccatgatccgacaatattaaccttcacaggaagaatagaatcatagggaaacgcatgaaatttgtgatctgataatttaaaaaacggggctaaaccagtaggagaagaagggtttggagtcttcccatctaacacggttaataaatctattccagttgctccagaagttagcagttgtgacaacccagaaaatctgacttctgttttaatgcggtgggtctgaagcatattccccaaatagggagatgtcaaaatagccatactccctccttaagaaacgataactgtcgcatcagaattaattgccttaacccaacccaccgtcggcggggttaaaacgaccatatttccttgatatatgatgtgcgcgttgttacccgttggaatactagggctatcgcacaaatacacagaaccgtgagtcacttgaagagttttcaaagatgttccgtctgagacttgtgtccatgtacttgcgtttggtatgatcttagcagttgccataatagacctcctttacaagtcagatctatttattatagacaaaaagaagcccgatcttcctgatcgggctatggttaaaccatcgtggggatggaaagactactcacagcaatttctggtattaggcgtttgggagagtgcgtcgggaatcgaacccgaaatcgtcaggatgctgaaagctatttgcatttcacttgcatatcctgcgactttcttaacttccttaaccccagattgttatgaagtcggcatgcacaccttcttcatcttcaggaagtcacactcattattctgattgcttcaactggattcgaaccagtgacctcaggcaccttgttctctcgaaactttcctgcgctctaccactggagctatgaagcaatttggcggaaggtgaaggattcgaaccttcgggggctaaaagccccactcgattagcagtcgagtcccttaaacctctcgggcaaccttccgtatactgcgcttcggtcaagaattttccaagccatgacctaaatctgggttcttgacctgaccgaaacgacttattggtgtgagtggatggatttgaaccaccgcgctgttacggggagatttacagtctcctgcattcgaccgctctgccacactcacgttgtttggtgctcgttagaggatttgaaccccagcactctggtgatcaacccagagagtcgcgcctcatccatttgttagcctaaacgagtcttgcaaaacccgcaccgaccgtctacaaacagactgcttgaccgagcgtaatatggacgggcttcacacccgattgtgcatttgatctgccgtagcccaactccccgcgcttatcagcgccgattggttaactggcaccagtgccttctggttcgatctcctggaactttacaggcttcgcgcccgaccatttctggaagttggggcatcgtttccgatgtggagtcaccccattttatacacagctggctgcaacgcttactgtgtttggtctaccaccacgtgctgggccacgcttgaggacatcggccatccttcctcagctaatccacgcggtggtagattttgtatcagagtgtccggtgtagctttccgtgaggtgcgcactactgtagcctctacactctgaatttggtgggcgttactttcgtcgggttcggattccgttcggctattggacgtcgttcgagagaacttgtcgttcgcgtacttcgtgcttacttgctttcgtttcacacccataattcataatttcctggataacattatcaaacaggaaatcctttaaccacttgatcccaaccacctgattatttttaggtggttccaccctcaaacgtaaatttttagggtattgcttcggatctaccaaaacttcaatcccagtttcaggatggtaaccataatagagatcaactttctcaccatggtatacggatttgcattttccgttgtagaaattagtcgtgcttgcgtcaagaaggtaggtattcttgtccatcatcaaggttttacattgcaccttgagcaaacgaccgttcacatcaatcacaaaatcatatggcgcattatctccgaaaggttgtgaaactgtgatcccgagttctgtacatttaaacaggaacctcgtttcacctatcatcccgcgctgttttgtgttcatgtgttcctgcaactattatttggcgacgatggcaggacttgaacctgcgacatacggtttaacagaccgccgttctaccgactgaactacatcgtctttatccgtgcttgttcgcttcgatcgcccttaattgggcgtccgcgtcagctttggtgtcatgcgtcccaagaactttagtcttggtgtggtcaaggacaacccacttatcgcctcgcttttcaacaaactcaacaatctctttgttgtcggtagcgagaccatcccactcagtaagaaaatcaccgaacgttttcatcatactcaccaattgtttattgaacaactggtttatttagtggtgaccctggctggacttgaaccagcgaccaatcgattatgagtcgactgctctaaccactgagctacggggccaaaatttggcggggtctgaggattttcacctctccttgctggtttggccgccagcctgttctctctggggactcgctgtccggacggctatcccttaaactatcgccccatattcaattgcaccagagcataattaccgctatacgaccaccctctcgggcaggttggttctgacccaacgtctctctgatgcgttttattgtgtgaccaaccaaggtctctcaaccctttatcccgtttgtgcatgatcagttcatgcatgcctgtttgtgcgcctcacggcaacgcgcttctgtgggtctttggcttgcaccgtagcctcaccacaggcgtttatttttccagggctttccacttgcaagtcttgaccatacagggatacgggcttgcaagatcgggttggacacaactttgatggctctggtactaaagatcccgcgtcacccatagacatggtttataaaatccagagccatcaaagttgcaaccaaatgttacaactcgcattcaatgtcttcgtcacacagaatacttgtcctctatcgctcggaccaaacgaggctggccgaagaaacaaaacaccactcaacactagagcgaggatgtggttgatagccacgatgtaatgtctctatcgaagagtgatcacacttgtcagatgatcactgttttgcgaactcgagaatactgcgcctgtctcgggtttccccttagttcggcctctcattgttcgtccggtcagggagctacccttgatgaccagcttatattgagatcttcgccagcgcccaccagtgtctagaatgtgtaggcgtcgaggtctgcaaggcattgccctatttcatacaacgctctctcaagtacgcaaatcgaagttctctgggcttaacaggttcgacccaacatgttgtatatcgcctaacttactcatgggacggccacttctgtgtacagaccttgagggttaaaagatatacccagagaacttcgatttgcgttgcccttcacttgccagagatcagggcaattggtaagacttcgctcacatcttagaggacactgcacgggactcgaacccgccacgagcatttggcgccaaatgcttccacctgtcaatgtcctctaagatgtggagccgttttagggagtcgaacccaaatgcttggccagcgctgaccttacatcccatctgacttaggcaacccacctctgcttactcaggagcttatcccctttggacagtcagatataacggcatcgtcgacgggagattattaatcacaccctcccgtctattttagtgcgccgaatttggtgatgaggaatcggaagacctcactggtgtatggctgttaagctactgccaggaacgtgttatcgtttgcatttatctttgtggtcagtttctaaaaacccgcaaagtcgcaagcaacgagaacgaaaacctgtttgagtcctaaatctacctgtcttggtctcacatcgctatgagtggccaggactcaaacagcttgccgttcagtgagttaactatactttgtaacccgttattgataaaaccttttatttttgttggagagggtgcggaatcgaaccaaacttctgagcgctaccccgtgtcaccaatgcaccatctcaccatcgatttgccggaacttacgcatccggcctcgcctgttggcgctcctaaaatccgggaagacagtaggaacaattatgttaccaacaggaaccttgttgccctttgacgcaacattatgaaccctctatgaagactcataatgttgtcggcgtgatggagattagcgcccccgacctccccttatatgttggtgtcgacttgccaacggggtgctctctgattctgagctaatcacgctacaacagggagtttcgttctccgacggtcaagtgcgtccactgacaccagcgaggtggatattgacttatggagccggagtcctcgtcctaggactgtgcttggcctcagcctttacaccgctggtcgcccgactctttacaccataaaatttactatactacagcccagctaatttgaaaagtcctctcccctttgagctacactgcccacagcagtgcggacgaaccttcactcaaaaccggatgcgccgaagtgggcaactgtttaccggactgatcgggtagacccaggagagagcaggtaatcccgatcaggaggatgaggagtaacatcatgtaaaacatagtgttccctcccttccatgcaactaactttactacaattatttattgaataaagcgttattttaacacttctaacttcacaacacgacgcagaaggtaaacatattcgccagcagcttcatgcgtcatataatgggtagaaggtgtggaatgattgttctgggtttcttttaatccaacacgttctgcccaaccattaatagagttcccatagaaaccgataatcgcgccaaaataaatgcgctggctctctggctctaaaacatagttgacagtgaagcaaatatcagaacgaatagactccagtttcataccaacacgaccttcggagaattcgttacgaacatataaatgacggaaacgatcgcgccgcgcttgtgatgattcttccaattcttgtcgaagtgaagaagttacgtcaatattaaatgaaaacgttggattctgtaaatcagaacgggccaactctaacgcagattcaacaacgcgacggaaattgatacttttacctgtcaggcgtttcacttcatctgtgattgttccaaaagggattttcgtggcgcgttccccttcgtattttgccaataaaggaataatgtgtttcatgattgctgtgttaatgtgcatttcggagcctcaccgaattgattaaatgtctccgttattatattggagatacgtttattgaatataaacacaagaatggccaataaaataatgacagtgattattaataggtaaaaactacttttctacaacatcaagaaactgtatcttaacgcggtgcccttcgagttgtgagtgttaacgaacaacgagaagcagcctccatggctgtggaatgggactgcctgtcaaggcggtgttacgagcgagggcgtcagccccctcgcgagtatattcgaataaattaaacgcgcgcgagcgttttgattcccgttggctaaatatcttcaacaccatattcaaactcaggtgatggagattatcatggccacgcaaacagttccttcactcgacgtcagagcatttgaatacattattaaacagcgaatgaaagctgatcccacctttaaagattatgactttgaaggttctggtcttagtgcgattattcgtttgttggcatctgacgcgaacgccatagcctttatgcaaaacatgcttaatggtgaaggccatttgaagacagcaaatcaacgttcaaacgtcggtttatctgctgcatttctttcctacacgcctgacaactatcgggcagcatacatgtatgtcaacatcaaagttacgccttatgacgccagcacagcacccaatgagatcatcatggatcggcgcgtaatgttcgttgggtcaaaagacggcagttcctacaactttactgttgagaaaccagtgtcagcaacgttgactgcagacggttattacatgttcaacaatgtaaagttggtacagggaaattggttgtacaagacatatgatgttgaaggaagcgcgatttcaacatacacgattccttctgggaatgtcgatatcaatcatatggtcgtgcaagtacaagaatcagagtcttccgacgtttcgacgacatatcaacgttacaacagtccatttgatttgagccaatatgcctacctgtatttcgtagaattgggtattgatggtctgtatgtgtttgaattcggagatggttatctttcgcgtcgagtagaagatgggaacgtcatattccttcaatacttggaaacttctggtgctgatgggaatgatatcacaagcctgtcatcagcgtcatccataggcgggtttaaccaggtcgatgtaaaattggtctccgaacgtagcgcaggtggggacgatccggaatctattgaggacactaaacggctggctcctttagcttatcaggctgatggtgctgcggttacagaaacagattatggtgtattgactgaaaggttattctccaacgtctcccgagctaaatcatatggtggtgacacgctgtctcctcctgattctggctatgtttacatcgctgttataccttctgttggtgagacactttcggacgctgagaaggccgatattgttgctgctctggacaagtataatgtgggatctattacccccaaagtggtagactccgagatcacatatatccaagtctcaacgactatattttgggatcctacttctacagtctacgttgaagaacagatgaaagttgttgttggaaacagtatcgttaaatggggtgaaaataatctcggcggatttgaccaattgtttgacaaagaaatattgcaggaagcaattacaaaaatggagcgttctatcaactcgaatattacttcagttggttataaacgccatttcaaacctgattatggagtcttagacagtttcacattcagttatggacgaagtatcaaacctggttctgtgaaaataacggggttcaaaccgcttcctgccgaggtggatttcacttactacatgcgcgacaataatggcgatctgaacatgtataaagtgaataacaatgacacgacaaaagagtttttagttcaaaaaacaggggttgtagattatgcaaatggggttgtagaccttcagcagatcacggtctctaactataaccctgaaggggtgaccatagtcgtgttaccggatggactgaaccagaacatacaggctacacaaaaccaagtattcaagattggtgatgtggttgttacgccagaggtgcgctatgtccaaagatcttaataatggacacaacggggttaagtatgaaactccgttgttctaccagaatgatttccctctgttcatagaatttatggacacgttcttcaattggctgtatagacagcaggggttcactcaagaagagatcttggcatatctggcagatacgtctagttggatgaatcccgagagtgaagattctccagttaaacagctgattgatttaaaggctgacaagaccccaggctcagaggccaaggattatttgtctgataaattcttggttcgtacctttgagaacatgatggctctggatgccgaagaacttttagatgccgacggaaggccgctactgtcaatagaagataagaacaaacagatagatgactggtataatgattttggtttccaacgaactgttgataaatcatttcaggaatttggtcattttatcccagttggttcggattctttattgacggccacgggcgatacgttctctgtttatatcgaaggaacgaaacggagaactctggatcatccgcgttggctcaaattgttaaaacatatctataaaatccgcggaaccaagaaagcaattgaattgttcttttggatatattttggttgtccagtcagcgtctattttacgaaagaagacataggcggtctggacggaaattttgaatgtgacggcacgacagggatgcgtgatgattactattatgatgaatacacttatgttataggagtcccaggcgacgtatctgattttgaaggtgtgtttgagcgcgtcttccgtcagcatttccatcctgctggatttactgtattcctagaaagcacaaggagctaaaaatggccgatttcctgagtcaatatacaggtcagcagatagatcagatattgggttctgttgatgacaaagtaagtaaaaatgacgttattaacgattttgaagttaacgacccattgtttccgccatctgctcgcctgacatacgagttgaaaagatctgttgacacaatcaatgatactttggccaacaaagttattcggacagatagtggtgaacaaacaatcaatggcaaaaagactttcaatgcgttgttggtgaccaatgggggcatacaagtccctgctggaaaatccgtttcaatcacagatgaaccaacgaactcgacagatggtgttaacctgagtatgttgagaaagcatggcgtcagtgaagacactaccccgcctggttctggtttggctggggagttgttgacatccggatattacaaattcaatttggatttcaccagattgtctgcgtataccggaccaacgttgccgacttcagttccagtacaaacagattctggagcgcattattggacaatgactgcattttttgatgcgttgtttgcagataaccgatttaatggatatgtgacaacgacagcgtatgacgcagatatgctgagtctagagtcccagattaataacatcaacacgtcgttgaacgggaaagtcaacacatcgacttacaacactaagatggcgtcgttggatttgagtatctccaatatcaatacgtctcttggtaataaagtcgaggtcagcacatataatacgaagatgacttccttggacggcagtatcagttctctcacgactggtaaggtcgatgtatcaacgtataatgccaaaatgaccacaattgataacagtttagggaacgcagtttatcgcacaaaaacaaacttgtcgtcatacacggtttcggcttctggaacgacggctcttccagattttgatttggtgaacctttgtcaaatactggtaactgcgcaatgggggacaggtaacgttgttgacacatatcgagtaaccataactcgtgacggaacgatcaaatcagagttgttggttcagaaaagtacgagcggtacagtgacgttcacaggggctgtggtgggtggtaaattgagaatttccgttgttaatgcaaatacgacaaccgcttgttctgtcgattattctattgcagcttctttctaaaactaaatacccctagatgacatgaatacatttaggggtattacaaatggccaacaaaccaacacagcctcttttccctttgggtttagaaacttctgagtcttcgaacataaaaggcttcaacaactccggcactattgagcattcccctggtgccgtaatgacatttcctgaagatactgaagttacaggtcttccatcttctgtgcgttacaatcctgatagtgatgaatttgaaggttattacgaaaacggtggttggttgtctttggggggtggtggaatacgctgggaaacgctccctcacgctccctctagcaatttgttagaaggtcgtggctatctcattaataataccacagggacatctacagtggttctcccttcccctacgcgtattggggattccgttactatttgtgatgcttatgggaaatttgccacttacccattgaccgtgtctccttctggaaataatttgtatggctctactgaagacatggctataacaactgataacgtatcagcaacgtttacttggtctggacctgaacaaggttgggttattacatccggcgtcggtcttggccaaggccgtgtctacagtcgtgaaatctttacacaaattttggcgtctgaaacaagtgctgtcactctcaatactccaccaacaatcgtggacgtgtatgctgacggaaaacgtcttgcggaatccaaatattcactagatgggaatgtaatcactttcagtccttctttgccagccagcacagaacttcaggtgattgaatatactcctattcaattgggcaatggcggtggttctggttcttctacgattacctgggtctataatgggggttcagcgattggcggtgaaaccgaaatcacgttagacgtcgttgttgatgatgttccggccattgatataaacggaagtcgccagtataaaaatctggggttcacattcgatccattaaccagtaaaatcactcttgcgcaagaactggatgcagaggatgaagttgttgtaattatcaatggaacgccaaacatctataatcaaattgattatactttgcgggaagtggctcgtgtaacaaatgttaaagatacagaggtcgtttattttagtgttggtgctgtgttaagcgggtataaagttatctatgataaagtaacacagagatcatattttattccagagctaccgactggaaccacggcagtcagccttagttcttcggctatacttgtgcattctgctggtagtgttgatctgggcgcattagctgtatctcgtgaagaatatgttacattgtctgggacatttgattctggtgctgtcatcaatactaaaaatgaattactcacccacacggatggtaagtatcgttgggatggggcattacctaaaactgtcgatgctggatcaacgcccgaaacatctgggggtattgggattggatcgtgggtgggtgttggtgatgcttctttaagatctgatttgaattcgacaaatggggctggaattgtaaaaactggaaatgggaatactgtacaagaaacatttgattttataaaaaatgttcaacaaacaaatctacatccttttgttccttcaggcaattttggttctggacttccttttgattcttcgttacaggtattcaagtataatgatgaatattactcatacaaaggacctattgtcccaggttcgatagttccggcaaccccatccgttgactcaaactggaaaaaagaacctgtttctgattacatagaaaattacggtggtggtgtgcatgtagctgatcagactcctgcaattatggcggctatatctgctggtgttcgccgtatcatatttcctaaaggtgaacttagaatgtctcagtgtgatattccagaatacacacaaggcctcacgtttgaagggcaaggtgccaatcaggcgtacaccggaacaacagtgatcaaaccgttaaatgataatcaaaattgtttatttaattctggcgtaaaccagcacggacaggattctataaaatttaaaaacattcaatttgacggagaatggcgttgcaaccatgcaatattgcatgagtctggagctggttggtactatgaaaaactgacaggtaaaaatttcaatacatgggttatctacgatgatcagggtttgactgtaatgcgtgacgtttataccgaagcaaccagttcagatctgacgaaaagagctacaggtggatgtatccgtatatactcagattctgtaatggataccgtaaactgttatggaggaagtgttccactatggattgctgctggcggaaaccgaattactaatatctgggcaaatggtgggtatgatgttggcgcaatacgtttgatgccaaagaacgacagtactaaccatatcaacactgccatgagcaatctctatatcggtgaaagtacaaacgaatcaggcactgctattcctgtattagaactacaaggtaactcaattcgtcgcgtatctggtgttcagatttcaaatgcacattttgtgcatgctgacttgactctcaatacaaacatggtgatgatacgaaccgataaagttgatggttttgtgatgtcatcttttgatgttcttggacgaggcgagtacagttctgcaagcagtcgcacatataatttcttaaatgcgacagattctaccggaatggctttttctgccggaagtgtaagaggtattaataataacccaatagttttatcatcagggtctaggttagctattgatgccgttgaattcgatgggtggagtacgaactctgattcaggtatagagaataaagctgctatacgttcatctggtgctggaacaaggtgctgtattggtagctcatgtgctttcaatgcttcgcaagcggatgcttacgctgtgtatgcaggggatgaatttgcagttgatatttctattcttaatcttaactatcctagtagcaatataataattcctgcaaatacaaattacagttacatgtataagcgaccaggcggaaaattaaccttagtcaacacattgacgatttgataacaagaacccatgattcataaattttattagttcagactaaataccccatattggtcatcctttatggggtattatcatgatttctcaattcaatcaaccacgcggctccacttctatagaagttaacaaacaatctatcgctcgaaatttcggtgtcaaagaagacgaagtcatttatttcactgctggtattgatctcagtggatttaaagtaatttatgatgagtctacccaacgggcttattctctccctttcggtattgtttcagggacaactgcaattagtctggatgaaagagctatcctcactcattccgctggctctgttgaccttggggaattagcagtaagccgcgaagaatatgtgactttacctggttcttttaatttcggccataccattaatgtgaaaaatgaacttattgttcacgatgataaaagatatcgatgggatggttctttgccaaaggtagttgacgctggttctactccagatagttcaggtggtgttggtttaggtgcatggttgagtgttggtgacgcagcatttagacaggaagccaacaaaaaattcaaatattcagtaaagttatctgattattctacattacaggatgcagcgacagcagccgtagatggattgcttattgatatcaattacaacttcacagatggtgagtctgtagattttggtggtaagattttaaccattaactgtaaggctaagtttattggagatggggctttaatctttaataatatggggccaggctcggtaattaatcaaccattcatggagagcaagactactccatgggtcattttcccgtgggatgctgatggtaaatggattacagatgctgcccttgttgctgcaacgctgaagcaatcaaagattgaaggctatcaacctggggtaaatgactgggttaaattccctggattagaggcattactcccacagaacgttaaagaccaacatattacagccactctagatattcgcagtgccagccgagtagaaataagaaatgctggtggtcttatggctgcttaccttttccgtagttgtcatcactgcaaggtaattgattcagatagcatcattggtggtaaagatggaatcattacctttgagaaccttagtggtgattggggattaggtaattatgttattggtggacgtgttcattatggttctggtagtggtgttcagttcctgagaaataatggtggtgaatcccacaatggtggagttattggtgttacatcatggcgagctggtgagtctggtttcaagacttatcagggttccgttggtggtggtactgcacgtaactataatctacagttcagggattctgttgcattgtctcctgtttgggatggttttgacttgggttctgacccaggtatggcaccagaaccggatagacctggggatttacctatatctgaatatccattccaccaactgcctaataaccatttggttgataatattcttgttatgaactcacttggtgttggtttaggtatggatggtcgtggtgggtatgtttctaacgttaccgtacaggattgtgctggtgcaggtatgcttgcacatacttacaaccgtgtattttctaacattacagttattgattgtaactaccttaattttgattctgaccaaattatcattataggtgattgtattgttaatgggattaaggctgctgggattaaaccacaaccatcaaatggtctggttatcagtgcaccaaactccacaataagtgggttggtcggtaatgttcctccagataaaattcttgttggtaacttacttgacccagtattaggtcagtctagagtcatcgggttcaatagtgatactgctgagttggctctacgtattaacaagctgtcagctactctggatagtggtgctttacgttcccatctgaacggttctgctggttctggttcggcatggactgagttaactgcactctctggttcaacccctaatgctgtatctctcaaggttaaccgaggagattataagacaactgagctacctatttctggtacagtattaccggatgaaggtgtgttggatattaatacaatgtccttgtacttagatgcgggtgcactgtgggctttgatacgactccctgatggaagtaaaacacgtatgaagttatctgtgtaataagtaacttatgccccgctttggcggggttcttattatacctttgttcataaatttattagttcagactaaataccccatattggcaaatcctttatggggtattttcaaatgactagaaatgtagaagaattatttggcggcgtaatcacagcacctcatcagatccctttcacgtataaatcaaatgttggtggagaaactttcctttccttgccgttctatcctgtcactggcgtagtcacaatcaacggtggtatgcaagttccgttagacaactttgaaatcgaaggaaatatgttgaatctcgggcgcgcattgtccaaaggcgatgttgtgtattgcttattcgataaaattctttcgccagaagatacagccaaaggtatccgcatatacaaatttcaggccgtaggaggtgaaaccgagttcactcctgatttcacatcttatggagtccaatctctttatatcggtggcgagtacaaaacacccgaaattgaatattcctatgacagcacgacaggaaaagtatctttgcaaactgcactgactgcaggtgtttgggtagtcgctgaaatgtccattaaacagaattacctggcgctctcttccgatagtggagccagcttggtggggacttcttcaggtaacaccgttcaagaagttcttaattcacattctggctctttccagactggggtaaaactccttagttctaatgacttaatcgtggatcagtctgttatccctaaccaactttatcgttgggatggtgcatttcctaaaactgtagctgctggctcatcacccgcatcaactggcggcgttggcaatggttcttgggttagtgttggtgatgcgacattaagaggtgagttaaataatgaaggggtaatcaatttctcccatgctgatacgtatggtaatgacagtgtaggagcgcatcttcaaaatgtcgtatacccaacagacgcccctttcaatgccgctacagatggtactacagacacaacagtagctattaaaagtgcaatagcgcactgtatatcaaagggtaaaaaattagtattaaatcatttgttcatgattacagatactctggtaatcagtgatgggttgcatgttgagtgtcttactagtgactcaggtgttaagtctgatgtaccagcaggtaaatttgcagtaaaaataacgggggccaattctggttggtttgggggtaaaatcctgggtaaaaacttaccagaatctacaacagtaaggcaggatggcgtactctttgatgagaatgctgaatactgctttattactggtacagaggttactggcttctttgcaaaaggtttgcatacctcagatgctgatggtgtaggctatggtatttacgataaagggtatggtactctcatctctaaatgttatgcaaactcaaagttctgtgtagctctgggtggtactgagggtcgcgtacttaagaaccgtataaccaataactaccttacatccggggaggctaaaccatggagctgggctagcaactattgggatgggatagtatccgagaatgctcacagatatgtcatcgcttttaatgatgtatcagcctgtggacagagtggaatttactttggtgggaatggtggttattcaacagataacatcatcgtaaataacactgtttacgcctgctggaaccgtggtattgatatgggtttgttttctgaaaaatcggcaacaaatgacgtactcagaaatatcatcaagggtaacaatacctataacaaccgagaaaataatatctggcttgcaggtgttagtaactgctcagtagttggtaatacatcgtggtttgacaccaattatgatgtaatttttgcaggttatccaggcggtcatatttgtattagtctagcttctggtgcaaatggggaagcttgtgtagggaacacaatcgactctaatacttgtattgaccctagaggtaatgcaggtataacagtacctactggggcaactggtaatgtttttggatcaggtaataatctatctcaagctggggctatttatatagcttcacctgacttgattacgtctaacagatttgaactggcagtcactggttcgttcacaccggtcctgcttcctgaaagcggaagtataaccctgtcgtcatcaagtacaggagtctttagggcaaccggcaatagaatagatttttccgtaacagtaaatgtatcttcaatatcatcgccaagtgggaatttaaatatcgcctatcttccaggaatgagtgggaaaacaagctcaacatcaatgtttattattgactattggaatgatttaacgttatcaagtggtgtaattccattggcatcattaaatttagaaaatcaagatcaaataacagtctaccgaactgatgggggaagggttctctatgacttttcttcattgatgaaatcaacatcatcgtttatattaaaaggttttgttgattttaactaataatttacccctctccctctctcaggagagggtttattagaaaaacaggagattctcattatgaatccgcaatttgctcaaccaaaaggttctacttcaaaagaatcgaacaaagatagtattgcccgtaaatttgggtgtaaaaagtcggaagtcgtttatgccaaagcgggacagtcattgtctggttataaagtgatctacgataagttgtctcaacgtgcctacgctctcccttctaacattggtgctgtaacggtcaccagtttggtcgacggcatcctcacacactctggaggcaccgtagatttgggtgcattggctgtgttaagggaagagtatgttactcttgttgaaaattttacttctggatttacgattcgtgtcaagaacgaagtcgtgagcgatggggtgagtctatatcgttgggatggtgctttacctaaaaccgttgcacctggctcaacacctgaaacaactggtggagttggtttaggtgcatggttgagtgttggtgatgcttctttgagaacacaactggcaaacggcgatggctcgttgattggaattcatccacagggaacgctaaataatgtattaactgtacgcacacctgaacagtacaatgcggtaggcgacggaatagctgacgacacttcaaaattaaaagaaatgctttcagatataaacaacgttcctgaaacacttcctgatgctgcggctgtaaactcatacatggagcaagtagcagtaaaaattgacctgaccaagttataccgtttcacggagacgctatacataccaccaggtgtttcaattgaaataccaacatcaaacttttttactcgtgagtgtaagcaaggattgttttatgacccagtagataagaatacagcagcaattagcttaatggtgtaccgtaaacagccagatggttcatacaaactcaataaagatgtagattattatcctactgggttagatattgataatggggatgctataacatgtgccagaaaaattgatattaataaccttaacctgattacagcaccgggtgtaaaggtcggtgttaaatggataggtggggcaggttgcactacaaaagggttatccattggggagaatacagggagcgatattacaacagcaagactgccaagagtaggattattgcagtcagcatcatggggttctattcatgaaaatctacgtatcctgtataaaactcagggcgcagtgttcattgactctaatggcggggctgctgtaaacaatgcttacatttctcgccttggtaatactaatggtgagttggaacaagcggtgtataaaccagcagggtttactgaggtaggtgatgttgccgtcactcagtttgcaggttcagaggttaagtttaatagcccaatcatcgaacaagcatccttcgactttgttcacgctggtcgggatactgattcctatggtttatttatggttgataaaccccatattgagtcatctggcgggaagaaaaaacatagcttctatcttatcaataccagttcgaatgttacgttgtcaggagtaggtctttcaggacaagaccctgatttggatagcatgtacttccttaaaaactgtccagaaactgccagaaatgttgttagaggacaaatgccaataagtggcgtaaaactcgttaggggtactggtaattaccccacattggtactcgactgtacaaatatgggaagtcaattccaatttggagaagttggtgacattttctatatcaaagatgttgttggtgtaaaagcagatactctttatatagacccagtgaatggtaataattataactgggggaccaatgggactaaacctatacgggaattaactaatattgcaaagatatgccagctttttagatgtaaaagtgtttatcttaatgctggagaatcagttattacatctaacacagaattgcctatggtggtgtttgaaggtcctggaagcttaaaagcaaattctggtagtagtttccttattaaagcaggtggcacattatcactaattggcctttctggaatatctacagatggtggtcatatgttcagggtgtccacagtagagaaggtaaatatacacacaaactgttcagttaatgctggtgcggcttatgttgttctaagtgaggttcaaggtaatattgaatacaggcaattattttattcagtaaactgttctaagtatattggtgcaactgctggacaaactatcgctggtattatggttaagactgcaacaagaccgacaggaatcgatgctgctccagttgatggtaatgtttctctaacctataaaattatagaatcataaaaaaccccgcttcggcggggtttttatttctatctgtttgttggctgtatactaaatatctctataaaatggcactcgatggagcatcaaaatgcaatctacaaatttaaaccgtcgcccgtattgggatgactggaatcctgggaaacgtttttcacggattctttttcgtccgatgcctatcaaggttcagactcgcgaactgaaccagatgcagactattcttcaggatcaaattgaaaaactaggcaaccatttgttcaaagatggttctatggttatccctggcggtctgacgattaccaatgccgcagtctctttgaaattcactttggctggtggtactgaattcactgatctggaaggtatttctgaactctacgttttggggaaggacaataatgccaaagcgcgtgtgttgtctcttgaacgtgatctatctgctcctgatacgatgctggctatccttgagatgactgaagcgggtaatgctgacggattccatgttaacgataatctgtatttccagacttacgacgtgaatgataatttcattcgtattggttatggtatcgcggctgctgtgactggttctatcgttgctcgtatgacgaaaggcgtttacttcattcgtgggatgttcctagacgttgatgacgcaactcttatcgttgataaaacttcgaacacgtcttcccatcgcgtcggatttaaagtcactgaaaccatcgtgactgaatctgaagacgaatccctttattcaaatgctcagggaacgattaactccaaagctccaggcgctcatcgtcttcgaatagatctggtcttatctcgatttgattatgatgctgtggttgaagactttgttgaactggccaaagttcgtgatggtaaaatccagtctatggtgactcagtccacctataatattctggaagacacgttggcccaacggacgtatgaaaccaacggcgattacaacgtttcaacgcaccagatcgacatccgcgaacacctgaaagtcaataacaatggcggggtattcaatgctgctgatggcggtgacgaaagtaaattcgtgtctgtaatgaaaccaggtatttcctatgttcgcggtcgccgtatcgaaaacgtcggggaagagttggtgatcgttgataaggcgcgggataccgatgtcctgaataacacccctgttgcggtggccacaggcaattatctggtcaccaagaattctaaaggtgtgccagtgatatcccgcaccgttcgatacaaattactgaatgcttcaggcgttacccaggctacagcactttgtatatcagctgaacgtaattcaacagaatttcgtctgtatatgcgcgacctggtcgttactggcgatgcatccactatcaccaaagtgtcttacgaagaaagtggtatcaccatgttctctgctgaacttgaatccaatcagtttaaccagagttctatgatagatctgatcttctctcttccggtattcggtgtcaagactttagcaccaactggctccgtggatatcaactacactgttctcagaacgtataaagtgacgttggacaacagcggcgcgggttctatttctgctccattgggatatagtttttccccagaattttcgttgtactctgcagcgaaatcagatggttcggaagcgcagttcgatatctctggttctttgtctttgactggttctccggtcggttcggcgctgcagatttctctgggcagtggtaatgctaaccagtcaatcaatttgctggcgctgatgattcgtaccacggccacaatcaaaacgaagaccatcactgaaacaactgaaacagtgacgttcacctcgcagactagccgccctttggcgaatcacgatggttggaaattggtatcggtcaaaaacgatactggcgcagacgtgacctccagcttcgtcctagatggcggacagcgtgatgcagggtattacaagtctaacttgttatccagcgctggagcaatctcagggacgtatacagtggtgtatcaatacttcgcccacagttccggtgatttcttctctgctgattcgtatacttcgatggattataaagatatcccgaattatacatcttctacatcaggcgcggtgtacggcctggcagatagtttggatttccgtccgaaaatcaccaacggaacttctgacacagatatggttcgtccaaacactgcagttattctggacacagaatattatctgccgcgtattgacgcgatttatctggcagataacggtgtgtttggtgtggctcgtggcatcagttcgaacaatctggcttctccagcaatcccagcgaacgccatgcgtttgtatgaattgttgatcccaccgtatacgccgaatattgatgatatccaaattcgcactattgacaatcgtcgttatacgatgcgcgatatcggtaaactggaaacccgtatttccaatgttgaatactatacctctctgtcacagttggaatcttcagcgatgacacagcaagtgttcgaccccatcacaggcaatccccgtttcaaaaatggtattgcagctgatccgttcaaagacttccggttgattgatgatttgtctgaagattggatgggttctatcgataccgaaaacggacgtctgcgcccgtttgtacaacaaaacgttgttgacatgactcctgttggctggaacaaagtgatggatgggatggtggtttgtaattacacgcctgaaatctcagtgaaccaagaatatgcgacgacgacgatcaacgttaacccgtatgcagtattcaattgggaaggtttcttgaagattaacccgaccacagactactggttcgaaaactattatgttgcgccgcgtattatcaatgaaacgatcaacacccgtggtactgtacaagaaggttcagtgtacggaacatggcgtactgtttctgtttctgatcgtgtttgggaaccgcatggcgctggtggtgtgtggtggggatatcgttaccgcacgactgtttctgcccgtgatgtcaccacatatacatacacggacaaaacaaccacgaccatgactggtgaacagattgtggagacgcaagttatcccatacatgcgcgagatcgatatttctttcgacgcatctgggctgcgacctttcactcgtatgtatgcgttcttctctggtcgtgacgtcaatctgtattgcaaaccgaatggtggtaatttcggtgacccaatcaccactgatgccaatggtgctgttaaaggtatattccgcgttccacagaacgatacaatcaagttcaacacaggcgacaacgtgttccgcttaacggatagccctgttgacagtaaatctgcggatgatacactgaccaatgcagaaattgttcataaatctttcggtaagaaacaaggtattcaaaagacctttgtcaacactcgtgtcctgggttacactgccagtactcgcaccgaaaccagcacttctgaagttgtggtcgaccaatggcgtgatccgatcgcccaatcgtttatggtggcgactaagaatggcggcgagtatatcgaaggcgtggaagtattcttctctaccaagtcacgtgatgttccgatcactctggaaattcgagagatggagaacggcttaccttctcacacagtcattactcgtaagactttgaacccatctgaagtgacgatctctacagactcttccggcggtacgaagttcacctttgattatccggtgtatctgcaagcatcgactgagtttgctatcgttttgttggcgaatactcaggattacaacgcgtatatcgcggaaatgggcaagaaaaaccttctgtccaacgaatatatcgccaaacaaccgtatacaggggtgttcttcacttcttcaaacggttctacatggtctccgaaccaaatggccgatatgaaattccgcatatatcgttgcaactttgcggctggtcagaacgttgtaacatttgatccgaagctcgggccaaaacaacgtccgttgggattgaacactctgaactgcgtaagcgggtcttctgttgtgactgtgtttgcacctggtcatggtttggttgctgggaacaatgtcactctttctgaattgacaggcggttgtggctttactcctgaacaacttaacaaaacgtttactgtgacagatgcgagctatacttcattcaagattgacgtcggtacggcagcagacagcaatggacaaatcggtggagataatgcttctttcttgggcaattatctggttgatatgttctatgccagcgttaccaattcggctctggaaggttcaattctgaaattggaataccgttatcgcgatgccacttcaaattctatgtctgattgggctgagtttgaaactgacactgacgtggcgttaccgactgaaggtatctatcgtcaggttggtgacttccagattcgtgctactatgactcgtagtgaaaacaacgtgtacacagcaccgatgattgatggcgatgacctgagcgtgatcttcaactcttatggcgtagatccttttgaagacgttttcaaatatgtcacaaaggacattggtttcgataacccatgttcgactgtgaaactgttcttcggggcgatgctcccatctcagtcttcgatgaaggtgcaggtgaaacttctacgagcagggcaagagatggatagcgtggcttgggaagacgtcaccccaacttcgcctctggttaacgacggttccacattctttgaatatgagtatgacaagactgtggcgagcaataacccgtttgttggcctgaaagtccgagcgcttgtacgggggaaccgcgttgctcctccatcattcaaagacttccgtcttattgctctggcataaataatgttgaaaggtgggcgtaaagcccaccaaaccaatagaggataaagatatgcgtggagtgaaagtacaggggcacgcatccatgttgagaagcagttcttgccctggcgcgattatttgcaccgatcacgccgctggcatggctgcattgcaagcccgtcaacgcaatgaaacccgtgaagccaccattgttgaacaatcaaaccagatatctaatctggaggcgacaatcaaaatgatggccgaaaagttggggatcgaaatccccgaaggaggtcagaatggcgacgcaagctgaaagaacggggcttgacatccgtggtatcttaaccgctgtcgtggcttctgcattggtctctgcggcatcattcctttggttcatgggcgggatggaaacgcgcgtgaatgtattggaacgagattccaacaagatggatcaagttcttcagaaagtgaatgacatgagtgaacgtatggcgatcatgaatactgacctcgcatatgtcaaacaaaacatggctgagttaaaactcaatagcagcagtctttcagatgatgttcgcacactgcggattactgtatctgatttgcagcaaaaggggaataacaatggccgtcaataagattcgagacaaaaaatcttttatgaattatgtcttgcgtaaattgggtgctcctgtgatccaaattaaccttgacagttcacaagtcgaagatgcagtcgatgatgctctgcagaaattttgggaatatcatcgtgatggtagccaagatgcgttcttcctgtaccaagttaaacaagaagatatcgacaagggatatatagaatttcccgatgatattgacgatgtgattgaagttatccctggacctcctattgagtcaatcggaaactgggcaactcctcaatggcaaatggctcaagcaatgcttgtccccaaagcagcgctggtttctattcgtctcattgattatgtttccatgcaacaacgcctgtcggatatcacaagcgtcttaaatgttcgccgtaattttgtgtacaagaagttccagcgacgcctttacccgcagtttgctgccattgttgatgaaactctggcttttcgttgttatcaaaatatcgaccccgaatcggaagaaaacgctgaagcatggaatgatatgtggttgaaagcgtatgcgactgcattggtcaagcgccgttgggcagaggtgcttaagaaggcaagaggcatccgtctccctggtggtatcgaactggatggtgatagtatgttcagtgaggctgagaccgagatagagcggttggaggaagaattgcgtactggtcagcaataccctatcgattttatgatgggataagagaaggcgggttatcccgcctttctttttataacttgatgtcgcgtccggaaggatctgaatttgctagaatataaccatcaaccacgttcttttcgtcccaccattcaataaaatcattacccttttcgtctttgataagacccacaatgttatatgaaccatcggcgattttagcatctacacagataattctcacacgccttccgtcacgagtttctttaatatcttcaacattcatttatcaattcctcaaaagaattaatgattgactaaataccttcaaaattgagggtaaagtcatggctacttcaaaatatttcaactatacagcacaccaaggtactcaaaaattaattgatgacttggtggtcgagatgatacaattgcgtgggattgacgtcaagtatattccacgttctattgttgaaaaatacccaattttaaacgaggccgaacacaaattcgaccaagcgtttgacatcgaagtgtacatgcaggattatcaaggcttcaacacacagatgtgggaaaagttcggcggtatccaattacaagatgaagtgaccttcactattgctcgtcgtcgtttttcagaagttattggtaatggtccaggccttgaacaaatgcctcaagaaggtgatttgatatatctgcctatggctaacaaaatatttaaagtgaataacccgaacaatgatgaagaattcatgcaatttgggaaatggtacacatattccctaccatgtacgttgttccaatacggtaacgaagatttcgatacaggtgtatctgaaatagacgatatcgataaacgattgcaggatctggaaggagacggtgtgtacaaagacgccagcctgcaaacggataaccaatttgcggacgaaatagagaaagatctttcgcccaataaaatgaagatagacttcggggaataatcatggccagaccatttgaaaaatatttctatcatgaatcattgttgaaatacatacatgtgttcaacgctatcatgtcagatttaaaagtcaaaaccgaacgtggcttgatggaaattccgctgcatatggccattggccgccgtaatgacctcaacaggaacgtgccagccaatatgctaccatttgcaacaatgtcctttggtcagttcgaaatcaataaacaggtgacgaagtcataccacaaccaaatatcgaccgctacggcacggtccaaacaacgcattccgatcattatagattttgaatacaacatcagaactaaaaaattggtcgaaatgttgcaagtattagaacaaatttattctgtgttcactccttctgttgactgtcagataaaggacaacgatactttatctcaagatcagaacgtgaagataatgttggtaaatcacacgatttctgacaactgggaaggggacgcaacagaatcaccacacatagattgttcattcaattttcaattacatggtcacatctatggagaagattattgggttgatgatggttcgggcggtggggatccaaacgtcatcaaagagataattattgaaatgtctaatgatttgaacatgccatggtcggaacttcctgagtggttccgagttgataaagacggcatacatcatccggaggattgatcatgagcaatatgtcagaaaggttactcgccacgcttgatgcagtgaccgccagagacgaagtcgggaaggaggcgatggaggctgtcgcccctcgtccaggtattgacttcgatgaaaatacaggtgaatggtttggtgaacgtcctgaagggtatcagccgattcctgaacctccttcattggaggatattgcttccaaggaatccaaagtacctgagtttgcggacactgatgctacaacggattacaaacggatacgcgacacaacatatgccatgcaagaagccacaatgttcatgatgggacaagccgctaaattggctgcatccacagaagctccgcgagcattttctgttttccgtgaattgggtgaactcatgcgcggtctgaataaagacctgatggaaaaccagaaaaccatcaaggcagtaacaggtgataaagaaccacccgttgatgacacgacagtggatgttacaacttcaccagacggaacaacaacagtatcggttgggaaaaaggcgcgatcttctcgcgatttattgaagacgatcgaagatgcacgccgccgcgctgaagaaagagcacaagcaaaggccgcacaacaaccagaagatgaaatcattgatggtgaaacagttgatgtgaaggaagaagacg

SEQ ID NO: 7 - protein, 767 aa, synthetic construct
MNPQFAQPKGSTSKESNKDSIARKFGCKKSEVVYAKAGQSLSGYKVIYDKLSQRAYALPSNIGAVTVTSLVDGILTHSGGTVDLGALAVLREEYVTLVENFTSGFTIRVKNEVVSDGVSLYRWDGALPKTVAPGSTPETTGGVGLGAWLSVGDASLRTQLANGDGSLIGIHPQGTLNNVLTVRTPEQYNAVGDGIADDTSKLKEMLSDINNVPETLPDAAAVNSYMEQVAVKIDLTKLYRFTETLYIPPGVSIEIPTSNFFTRECKQGLFYDPVDKNTAAISLMVYRKQPDGSYKLNKDVDYYPTGLDIDNGDAITCARKIDINNLNLITAPGVKVGVKWIGGAGCTTKGLSIGENTGSDITTARLPRVGLLQSASWGSIHENLRILYKTQGAVFIDSNGGAAVNNAYISRLGNTNGELEQAVYKPAGFTEVGDVAVTQFAGSEVKFNSPIIEQASFDFVHAGRDTDSYGLFMVDKPHIESSGGKKKHSFYLINTSSNVTLSGVGLSGQDPDLDSMYFLKNCPETARNVVRGQMPISGVKLVRGTGNYPTLVLDCTNMGSQFQFGEVGDIFYIKDVVGVKADTLYIDPVNGNNYNWGTNGTKPIRELTNIAKICQLFRCKSVYLNAGESVITSNTELPMVVFEGPGSLKANSGSSFLIKAGGTLSLIGLSGISTDGGHMFRVSTVEKVNIHTNCSVNAGAAYVVLSEVQGNIEYRQLFYSVNCSKYIGATAGQTIAGIMVKTATRPTGIDAAPVDGNVSLTYKIIES

SEQ ID NO: 8 - protein, 796 aa, synthetic construct
MTRNVEELFGGVITAPHQIPFTYKSNVGGETFLSLPFYPVTGVVTINGGMQVPLDNFEIEGNMLNLGRALSKGDVVYCLFDKILSPEDTAKGIRIYKFQAVGGETEFTPDFTSYGVQSLYIGGEYKTPEIEYSYDSTTGKVSLQTALTAGVWVVAEMSVKQPNISPLFDRSIQEIARSANVKDSEVIVSTDTISLLDGKKVVYDIATQTSYGLPTIPDGSVISSVSGGKLNYNPGDVQVNLLPLPNSAALLRGELNNEGVINFSHADTYGNDSVGAHLQNVVYPTDAPFNAATDGTTDTTVAIKSAIAHCISKGKKLVLNHLFMITDTLVISDGLHVECLTSDSGVKSDVPAGKFAVKITGANSGWFGGKILGKNLPESTTVRQDGVLFDENAEYCFITGTEVTGFFAKGLHTSDADGVGYGIYDKGYGTLISKCYANSKFCVALGGTEGRVLKNRITNNYLTSGEAKPWSWASNYWDGIVSENAHRYVIAFNDVSACGQSGIYFGGNGGYSTDNIIVNNTVYACWNRGIDMGLFSEKSATNDVLRNIIKGNNTYNNRENNIWLAGVSNCSVVGNTSWFDTNYDVIFAGYPGGHICISLASGANGEACVGNTIDSNTCIDPRGNAGITVPTGATGNVFGSGNNLSQAGAIYIASPDLITSNRFELAVTGSFTPVLLPESGSITLSSSSTGVFRATGNRIDFSVTVNVSSISSPSGNLNIAYLPGMSGKTSSTSMFIIDYWNDLTLSSGVIPLASLNLENQDQITVYRTDGGRVLYDFSSLMKSTSSFILKGFVDFN

SEQ ID NO: 9 - protein, 921 aa, Kuttervirus CBA120
MTRNVEELFGGVITAPHQIPFTYKSNVGGETFLSLPFYPVTGVVTINGGMQVPLDNFEIEGNTLNLGRALSKGDVVYCLFDKILSPEDTAKGIRIYKFQAVGGETEFTPDFTSYGVQSLYIGGEYKTPEIEYSYDSTTGKVSLQTALSAGVWVVAEMSVKQPNISPAFDRSIQEIARSANVKDSEVIVSTDTISLLDGKKVVYDIATQTSYGLPTIPDGSVISSVSAGKLNYNPGDVQVDLLPLEDSFINVINTLGRNDGAKYIGECHSVADLRNTEPTMDGQRIILKQHTAGTLLGGGVFRALIDGTGKTDNNGTVIKTVGGAAWLRVNADRVNPFMFGALGGSNDDTIPVQSCVDSGKATQLTDAHYVSNIQLKYNTSSIYGSGLHYSRLHQLPSATGNCITIKDTCSLIVLDAFGVYGTGAQQGTSFTAGTTGIYVETPSGLSADYPFHTTADPRRDLCISKVHIAGFDEYGLNIDSGNFSVTTDSLLVNHINQVGVRCATTDWTWTNIQVNTCGKQCLVLDGCGNGRIIGGKFIWANWQPYGTVGQFPGITINNSQNMVINGIEVQDCGGNGIEISESYSISMNGLNTNRNGINANNTFYNIVFNKSDAVINGFVGLNYAANSGSGANSSAGNFQFLSNDCSVTINGVVETGYMGINFIGDNNIINPTNSDLSINGLVNYSKTGLQTMNETPTFDGVSTTPVYVSVPSSVGQVNGLRLSQANKDKLLYSRTAGPEGITMAAVIVPTISGAEVFNFMAIGSGFSDTSNSLHLQLVIDASGKQTIALLLGGDGTTQILSGDLPNDLKLQSGVPYHIAIGAKPGYFWWSILNIQTGKRIRRSFRGAYLAVPFNSIFGLTSSLTFFSDSNAGGDACSGVGAKVYVGMFSSENDYVASRYYNLINPVDPTKLISYRILDSSI

SEQ ID NO: 10 - protein, 724 aa, synthetic construct
MTRNVEELFGGVITAPHQIPFTYKSNVGGETFLSLPFYPVTGVVTINGGMQVPLDNFEIEGNTLNLGRALSKGDVVYCLFDKILSPEDTAKGIRIYKFQAVGGETEFTPDFTSYGVQSLYIGGEYKTPEIEYSYDSTTGKVSLQTALSAGVWVVAEMSVKQPNISPAFDRSIQEIARSANVKDSEVIVSTDTISLLDGKKVVYDIATQTSYGLPTIPDGSVISSVSAGKLNYNPGDVQVDLLPLPNSAALLREELGSPTGTNLVNFKNSAVGALVRSVTSKLEDNPISVKDYGCKGDGVTDDTAGLQLAVAGALSGLVIYFPKGIYLTSLPLNFTKPVTLIGEKGARIELTGGDHDYVIQFDFRDTDLMYWGYEAYMSNIIVDGKGFAKDGFSLRGVISSRFDNLRATNISRAGLHCWWTQCNLYNNFHCSKNVELFTTTPKYGILIDYEEGSPNKRGTSADTFINPVCEHTTEAGIEATFCINTIFMNGTSEGCRFGIAFGHESDLEQTAVTNTVIGMDMEVNSDGDVLLRKTATSNLFLGVMAGYSSPSSQIQGAEFNRWIGGSSSGFHFYGGTKGNKIEGTMLLGASATILDDDKTNDWDSLYNISNATYTGSTNPVNKRVQHDGTGTVTIDPMKCSLATINATSSTITINTLDNARDGMRMDMIIYNTSGQPLTVNWGDSFRIGGFVPPESGKQKGLSFVWDANFAHWYCVGMSADGLVY

SEQ ID NO: 11 - protein, 627 aa, Kuttervirus CBA120
MISQFNQPRGSTSIEVNKQSIARNFGVKEDEVIYFTAGIDLSGFKVIYDESTQRAYSLPFGIVSGTTAISLDERAILTHSAGSVDLGELAVSREEYVTLPGSFNFGHTINVKNELLVHDDKKYRWDGSLPKVVAAGSTPDSSGGVGLGAWLSVGDAALRAELNTKVSDGTFPATIKYKYGLPSVIDGAIYRTVQDKLDDFVFLEDFGGKDDAGSTDNSIAFRKAFASGARKIRLRGSGVYGMATRDIELPAKYEIIGNAKNPEIKYLGTDTSFTMFTLTGSGPASNQWKQGGMFRDLIISSDVKINWMLGRHVQNLDYDRVFFYNSATVLNNYHYVNFTRCERWGSAFIGRADLNTIQFISESPKFHLCFSSGSPIDVWDTADLAITKCTMFAGDYAVRTRVTQKQVTAPDLFAGYPVLITCSVFDAVRGHAWDLEGSVYSTITGNLVSAGRDTNSHGAYIKGGRSLSLTGNVFTYCGNYGLVLEDVQQSGFVGNVFNGNKTGGLGTLACKDLSIVGGSMGTTYVRGGYYTQPVGYSDISSNSTGILLSGVAFDEALTTKVYLDTSITTRNKVINCSGVPDTIARGSTANRPANPQASYQYYDTTLGIPIWWNSVSGTWKNAAGADV

SEQ ID NO: 12 - protein, 708 aa, synthetic construct
MISQFNQPRGSTSIEVNKQSIARNFGVKEDEVIYFTAGIDLSGFKVIYDESTQRAYSLPFGIVSGTTAISLDERAILTHSAGSVDLGELAVSREEYVTLPGSFNFGHTINVKNELLVHDDKKYRWDGSLPKVVAAGSTPDSSGGVGLGAWLSVGDAAFRQEANKKFKYSVKLSDYSTLQDAATAAVDGLLIDINYNFTDGESVDFGGKILTINCKAKFIGDGALIFNNMGPGSVINQPFMESKTTPWVIFPWDADGKWITDAALVAATLKQSKIEGYQPGVNDWVKFPGLEALLPQNVKDQHITATLDIRSASRVEIRNAGGLMAAYLFRSCHHCKVIDSDSIIGGKDGIITFENLSGDWGLGNYVIGGRVHYGSGSGVQFLRNNGGESHNGGVIGVTSWRAGESGFKTYQGSVGGGTARNYNLQFRDSVALSPVWDGFDLGSDPGMAPEPDRPGDLPISEYPFHQLPNNHLVDNILVMNSLGVGLGMDGRGGYVSNVTVQDCAGAGMLAHTYNRVFSNITVIDCNYLNFDSDQIIIIGDCIVNGIKAAGIKPQPSNGLVISAPNSTISGLVGNVPPDKILVGNLLDPVLGQSRVIGFNSDTAELALRINKLSATLDSGALRSHLNGSAGSGSAWTELTALSGSTPNAVSLKVNRGDYKTTELPISGTVLPDEGVLDINTMSLYLDAGALWALIRLPDGSKTRMKLSV

SEQ ID NO: 13 - protein, 1036 aa, Kuttervirus CBA120
MANKPTQPLFPLGLETSESSNIKGFNNSGTIEHSPGAVMTFPEDTEVTGLPSSVRYNPDSDEFEGYYENGGWLSLGGGGIRWETLPHAPSSNLLEGRGYLINNTTGTSTVVLPSPTRIGDSVTICDAYGKFATYPLTVSPSGNNLYGSTEDMAITTDNVSATFTWSGPEQGWVITSGVGLGQGRVYSREIFTQILASETSAVTLNTPPTIVDVYADGKRLAESKYSLDGNVITFSPSLPASTELQVIEYTPIQLGNGGGSGSSTITWVYNGGSAIGGETEITLDIVVDDVPAIDINGSRQYKNLGFTFDPLTSKITLAQELDAEDEVVVIINGTPNIYNQIDYTLREVARVTNVKDTEVIYFSVGAVLSGYKVIYDKVTQRSYFIPELPTGTTAVSLSSSAILVHSAGSVDLGALAVSREEYVTLSGTFDSGAVINTKNELLTHTDGKYRWDGTLPKTVAAGSTPATTGGVGSGAWLSVGDASLKSNLNKPNGLSYIGTVSSVSELSSIAGLIGDSIILDSYVDGFNLGGGVMVAVNSDTVVDNIVTFQGNGVVWKRKLFNGVADVYEAGYTGTGDLAIFINKINAVGFDCIVPVSGEITTPIIFDIAKGALIGKNKCTLIESASATGDYYLTIVNTDTDYTNRDVINATALMTGVSFVGKGTRKLAIGGSTSGEVSELRISNCGFISTAGIEFLDNAYRILFDKCALSRSFTNSVIFNSPANSGEVIKFNHCWMVDNGGPFTFKNGQFIFDSCSLPAGKKSGYFDPVVALSDNATTVFTNGNIEYQPGQSFVGFTVDGSSRLSISDSTILLPNDYSTVPIVNNGDGVVSLNNCSLPLYGSTTIATGFATRQLIGGLSKKIMSRGCYPRAGFITSNWNLGCIVSPYINSVSNGSGQFENISNWTLSQTGTDVVTVTTGNDVPNDLMFSTSFVLSVPTVGAAANFTQTIIDCEPGRYFQLGFWAKNTTTTLASIRFLDQQGNAVADSIGYNIPVGNTFNFYALVDCVPPGAYKAEINFNVSSIVGGIAIHNVIYGLI

SEQ ID NO: 14 - protein, 1036 aa, synthetic construct
MANKPTQPLFPLGLETSESSNIKGFNNSGTIEHSPGAVMTFPEDTEVTGLPSSVRYNPDSDEFEGYYENGGWLSLGGGGIRWETLPHAPSSNLLEGRGYLINNTTGTSTVVLPSPTRIGDSVTICDAYGKFATYPLTVSPSGNNLYGSTEDMAITTDNVSATFTWSGPEQGWVITSGVGLGQGRVYSREIFTQILASETSAVTLNTPPTIVDVYADGKRLAESKYSLDGNVITFSPSLPASTELQVIEYTPIQLGNGGGSGSSTITWVYNGGSAIGGETEITLDVVVDDVPAIDINGSRQYKNLGFTFDPLTSKITLAQELDAEDEVVVIINGTPNIYNQIDYTLREVARVTNVKDTEVVYFSVGAVLSGYKVIYDKVTQRSYFIPELPTGTTAVSLSSSAILVHSAGSVDLGALAVSREEYVTLSGTFDSGAVINTKNELLTHTDGKYRWDGALPKTVDAGSTPETSGGIGIGSWVGVGDASLKSNLNKPNGLSYIGTVSSVSELSSIAGLIGDSIILDSYVDGFNLGGGVMVAVNSDTVVDNIVTFQGNGVVWKRKLFNGVADVYEAGYTGTGDLAIFINKINAVGFDCIVPVSGEITTPIIFDIAKGALIGKNKCTLIESASATGDYYLTIVNTDTDYTNRDVINATALMTGVSFVGKGTRKLAIGGSTSGEVSELRISNCGFISTAGIEFLDNAYRILFDKCALSRSFTNSVIFNSPANSGEVIKFNHCWMVDNGGPFTFKNGQFIFDSCSLPAGKKSGYFDPVVALSDNATTVFTNGNIEYQPGQSFVGFTVDGSSRLSISDSTILLPNDYSTVPIVNNGDGVVSLNNCSLPLYGSTTIATGFATRQLIGGLSKKIMSRGCYPRAGFITSNWNLGCIVSPYINSVSNGSGQFENISNWTLSQTGTDVVTVTTGNDVPNDLMFSTSFVLSVPTVGAAANFTQTIIDCEPGRYFQLGFWAKNTTTTLASIRFLDQQGNAVADSIGYNIPVGNTFNFYALVDCVPPGAYKAEINFNVSSIVGGIAIHNVIYGLI

SEQ ID NO: 15 - protein, 770 aa, Kuttervirus CBA120
MNEMFSQGGKGSTGILTNKQAVARHFGVKQSEVVYFSVGVDLGGYKVIYDKETQRAYSLPVGIASGTTAVSLSTAAVLVHSAGSVDLGSLAVSREEYVTLPGSFDSGSTLNVKNELLTYTDGKYRWDGILPKTVAPGSTPASTGGVGLGAWISVGDASLRTQLANGDGSLIGIHPQGTLNNVLTVRTPEQYNAVGDGIADDTSKLKEMLSDINNVPETLPDAAAVNSYMEQVAVKIDLTKLYRFTETLYIPPGVSIEIPTSNFFTRECKQGLFYDPVDKNTAAISLMVYRKQPDGSYKLNKDVDYYPTGLDIDNGDAITCARKIDINNLNLITAPGVKVGVKWIGGAGCTTKGLSIGENTGSDITTARLPRVGLLQSASWGSIHENLRILYKTQGAVFIDSNGGAAVNNAYISRLGNTNGELEQAVYKPAGFTEVGDVAVTQFAGSEVKFNSPIIEQASFDFVHAGRDTDSYGLFMVDKPHIESSGGKKKHSFYLINTSSNVTLSGVGLSGQDPDLDSMYFLKNCPETARNVVRGQMPISGVKLVRGTGNYPTLVLDCTNMGSQFQFGEVGDIFYIKDVVGVKADTLYIDPVNGNNYNWGTNGTKPIRELTNIAKICQLFRCKSVYLNAGESVITSNTELPMVVFEGPGSLKANSGSSFLIKAGGTLSLIGLSGISTDGGHMFRVSTVEKVNIHTNCSVNAGAAYVVLSEVQGNIEYRQLFYSVNCSKYIGATAGQTIAGIMVKTATRPTGIDAAPVDGNVSLTYKIIES

SEQ ID NO: 16 - protein, 798 aa, Kuttervirus Det7
MTRNVESIFGAVVTAPHQIPYTYTATGGETFISLPFYPVTGFITINGGVQVPVDNYEIDGNTVNLGRALEADDVVYCLFDKILSPEDYENGIRIYKFQAVGNETTFTPDFTTYGVQTLYIDGKFQVPGVDYNYNSATGVVSFLNGSPTAGVWVVAEMSIKQNYLALSSDSGASLVGTSSGNTVQEVLNSHSGSFQTGVKLLSSNDLIVDQSVIPNQLYRWDGAFPKTVAAGSSPASTGGVGNGSWVSVGDATLRGELNNEGVINFSHADTYGNDSVGAHLQNVVYPTDAPFNAATDGTTDTTVAIKSAIAHCISKGKKLVLNHLFMITDTLVISDGLHVECLTSDSGVKSDVPAGKFAVKITGANSGWFGGKILGKNLPESTTVRQDGVLFDENAEYCFITGTEVTGFFAKGLHTSDADGVGYGIYDKGYGTLISKCYANSKFCVALGGTEGRVLKNRITNNYLTSGEAKPWSWASNYWDGIVSENAHRYVIAFNDVSACGQSGIYFGGNGGYSTDNIIVNNTVYACWNRGIDMGLFSEKSATNDVLRNIIKGNNTYNNRENNIWLAGVSNCSVVGNTSWFDTNYDVIFAGYPGGHICISLASGANGEACVGNTIDSNTCIDPRGNAGITVPTGATGNVFGSGNNLSQAGAIYIASPDLITSNRFELAVTGSFTPVLLPESGSITLSSSSTGVFRATGNRIDFSVTVNVSSISSPSGNLNIAYLPGMSGKTSSTSMFIIDYWNDLTLSSGVIPLASLNLENQDQITVYRTDGGRVLYDFSSLMKSTSSFILKGFVDFN

SEQ ID NO: 17 - protein, 875 aa, Christensenvirus TR2
MAIRTVNSCCGNDGALVEHFIGTAYDVVKNVYDNLGILQYIYDFLNQHGVLVTVDSVDELKALNTDMKYARVYTYSTAAGYGYTDYLYVDDDTSGIIPNDATATGTWIEVASSTSLNNGSAYIPYIYNNGSAIGGETTIAVPNDTIGVPYIIKNHGTYIVTLGYTYDPITLTVTLDDALAIGDEVILLLSGTPAVPTDPTVSDWVQINWLYNGGYAVGGEQVIAVPYNFASVPAIYKNGLRYYAGLAAQSYMIDVENQRVLLTEPLAPNDRLIVQLGGEVQTFIMTDRTVQEVARSANVHENEVILSTNTTQYLNGMKIIYDVVAQKIYGLPTLPTNVYINSVSNGQLTYSPGNITVELLPMPDAELDAFKNTLASSNGYTYIGGLSERFSLPNKWVVVDNAPYYGNLELAVTSNSSNTAFLLGKGKSYDFLTFRTARNTKKNLTFFGSGVPELASDKRSFVPGTGTIIRGPMKTEVSGWQLVNLGIDCGAQTSVDLFGGEWDDPIQCYGLGNNANVFIDNVKTLSHTNLSGPNYPGTHSLLLELLDGAYVGKIELIGGFHGLTLKGTNIDVMDAHCYGQRGDAFIIKSDSAATAADIYIGRIKVGLRNNSGFTDVTMGGIYDAHDSVNIDNVEIGSLQVYNASWGLIPSDANTGYISNLRINSYVAMNVYGNYYSLVIDGRCVGVNIGEHQCSNTSGGISIDPNAVNVTLGNGFSKGSTKSGYALGSNSLVHGNLIADENGEFGVDYKGGVGFDASKITGYSNTFGLVSAVPTALNGNPPQNGWADTGAFMVQLTGKTVTVSGSMVVGTGAVAYTTLDICRPKKRIPIAAWGVAGSTMIPVECYIETNGNLNVVGYASITAGNQIHFSGQYLIY

SEQ ID NO: 18 - protein, 994 aa, synthetic construct
MANKPTQPLFPLGLETSESSNIKGFNNSGTIEHSPGAVMTFPEDTEVTGLPSSVRYNPDSDEFEGYYENGGWLSLGGGGIRWETLPHAPSSNLLEGRGYLINNTTGTSTVVLPSPTRIGDSVTICDAYGKFATYPLTVSPSGNNLYGSTEDMAITTDNVSATFTWSGPEQGWVITSGVGLGQGRVYSREIFTQILASETSAVTLNTPPTIVDVYADGKRLAESKYSLDGNVITFSPSLPASTELQVIEYTPIQLGNGGGSGSSTITWVYNGGSAIGGETEITLDVVVDDVPAIDINGSRQYKNLGFTFDPLTSKITLAQELDAEDEVVVIINGTPNIYNQIDYTLREVARVTNVKDTEVVYFSVGAVLSGYKVIYDKVTQRSYFIPELPTGTTAVSLSSSAILVHSAGSVDLGALAVSREEYVTLSGTFDSGAVINTKNELLTHTDGKYRWDGALPKTVDAGSTPETSGGIGIGSWVGVGDPDAELDAFKNTLASSNGYTYIGGLSERFSLPNKWVVVDNAPYYGNLELAVTSNSSNTAFLLGKGKSYDFLTFRTARNTKKNLTFFGSGVPELASDKRSFVPGTGTIIRGPMKTEVSGWQLVNLGIDCGAQTSVDLFGGEWDDPIQCYGLGNNANVFIDNVKTLSHTNLSGPNYPGTHSLLLELLDGAYVGKIELIGGFHGLTLKGTNIDVMDAHCYGQRGDAFIIKSDSAATAADIYIGRIKVGLRNNSGFTDVTMGGIYDAHDSVNIDNVEIGSLQVYNASWGLIPSDANTGYISNLRINSYVAMNVYGNYYSLVIDGRCVGVNIGEHQCSNTSGGISIDPNAVNVTLGNGFSKGSTKSGYALGSNSLVHGNLIADENGEFGVDYKGGVGFDASKITGYSNTFGLVSAVPTALNGNPPQNGWADTGAFMVQLTGKTVTVSGSMVVGTGAVAYTTLDICRPKKRIPIAAWGVAGSTMIPVECYIETNGNLNVVGYASITAGNQIHFSGQYLIY